(19) United States
(12) Patent Application Publication
(10) Pub. No.: US 2024/0270762 A1
(43) Pub. Date: Aug. 15, 2024

(54) PENICILLIN-BINDING PROTEIN INHIBITORS

(71) Applicant: Venatorx Pharmaceuticals, Inc., Malvern, PA (US)

(72) Inventors: Christopher J. BURNS, Malvern, PA (US); Denis DAIGLE, Street, MD (US); Guo-Hua CHU, Exton, PA (US); Jodie HAMRICK, New Holland, PA (US); Steven A. BOYD, Chester Springs, PA (US); Allison L. ZULLI, Chesterbrook, PA (US); Stephen M. CONDON, Glenmoore, PA (US); Cullen L. MYERS, Phoenixville, PA (US); Zhenrong XU, Chalfont, PA (US); Tsuyoshi UEHARA, Berwyn, PA (US); Nathan LINE, Royersford, PA (US)

(21) Appl. No.: 18/563,292

(22) PCT Filed: Mar. 25, 2022
(86) PCT No.: PCT/US2022/021883

§ 371 Date: Nov. 21, 2023

Related U.S. Application Data
(60) Provisional application No. 63/284,572, filed on Nov. 30, 2021.
(60) Provisional application No. 63/193,326, filed on May 26, 2021.

(51) Int. Cl. C07F 5/02; A61K 9/00; A61K 9/20; A61K 9/48; A61K 31/69; A61P 31/04; C07F 9/6596

(52) U.S. Cl. CPC C07F 5/025; A61K 9/0053; A61K 9/2013; A61K 9/2018; A61K 9/2054; A61K 9/2059; A61K 9/4825; A61K 9/4858; A61K 31/69; A61P 31/04; C07F 9/6596

(57) ABSTRACT

Described herein are certain boron-containing compounds, compositions, preparations and their use as modulators of the transpeptidase function of bacterial penicillin-binding proteins and as antibacterial agents. In some embodiments, the compounds described herein inhibit penicillin-binding proteins. In certain embodiments, the compounds described herein are useful in the treatment of bacterial infections.

### CROSS-REFERENCE

This application claims the benefit of U.S. Provisional Application Ser. No. 63/193,326 filed May 26, 2021 and U.S. Provisional Application Ser. No. 63/284,572 filed Nov. 30, 2021 which are hereby incorporated by reference in their entirety.

### STATEMENT AS TO FEDERALLY SPONSORED RESEARCH

This invention was made with government support under 1R01AI141239 by the National Institutes of Health (NIH), IR01AI160269 by the National Institutes of Health (NIH), Federal Award 6 IDSEP16030-01-02, subaward 4500003206, awarded by the Health and Human Services Office of the Assistant Secretary for Preparedness and Response (HHS/ASPR) under the CARB-X Pass Through Entity, contract HDTRA117C0070, awarded by the Defense Threat Reduction Agency (DTRA) of the Department of Defense, and contract 75N93020C00016, awarded by the National Institute of Allergy and Infectious Diseases (NIAID), National Institutes of Health (NIH). The government has certain rights in the invention.

### BACKGROUND OF THE INVENTION

Antibiotics are the most effective drugs for curing bacteria-related infectious diseases clinically. They are incredibly valuable therapeutic options that are currently losing efficacy due to the evolution and spread of drug resistance genes, leading to multidrug resistance bacterial organisms. Among the different classes of antibiotics, the penicillin-binding protein-targeting beta-lactams (e.g. penicillins, cephalosporins, and carbapenems) are the most widely used antibiotic class because they have a strong bactericidal effect and low associated toxicity.

Penicillin Binding Proteins (PBPs) are a family of essential bacterial enzymes involved in the synthesis of peptidoglycan, the major structural polymer found in the bacterial cell wall. Beta-lactam antibiotics bind with high affinity to PBPs and inhibit their transpeptidase function, resulting in disruption of peptidoglycan cell wall synthesis and rapid cell lysis of actively dividing bacteria. As there are no close mammalian homologues to PBPs, and beta-lactams are well-regarded for their safety and efficacy, PBPs represent an ideal target for antibacterials.

### SUMMARY OF THE INVENTION

Described herein are compounds that inhibit the activity of penicillin-binding proteins, the bacterial enzyme class targeted by the beta lactam antibiotics and do provide significant antibacterial activity in vitro.

Provided herein are compounds of Formula (Ia) or (Ib), or a pharmaceutically acceptable salt, solvate, stereoisomer, tautomer, N-oxide, dimer, or trimer thereof:

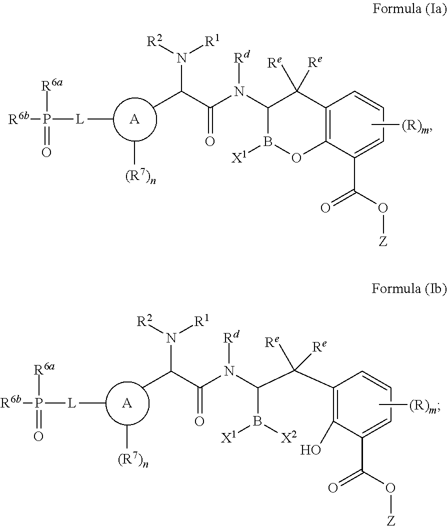

wherein:
R¹ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R² is hydrogen, C₁-C₆ alkyl, —C(═O)R³, —S(═O)₂R³, —C(═O)N(R⁴)₂, or —S(═O)₂N(R⁴)₂;
R³ is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{3a};
each R^{3a} is independently deuterium, halogen, —Y—CN, —Y—NO₂, —Y—OH, —Y—OR^{a}, —Y—OC(═O)R^{a}, —Y—OC(═O)OR^{b}, —Y—OC(═O)NR^{c}R^{d}, —Y—SH, —Y—SR^{a}, —Y—S(═O)R^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, —Y-cycloalkyl, —Y-heterocycloalkyl, —Y-aryl, or —Y-heteroaryl; wherein the alkyl, alkenyl, alkynyl, cycloalkyl, heterocycloalkyl, aryl, and heteroaryl is optionally substituted with one or more R^{3b};
or two R^{3a} on the same atom are taken together to form an oxo;
each R^{3b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{3b} on the same atom are taken together to form an oxo;
each R⁴ is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{4a};
each R^{4a} is independently deuterium, halogen, —Y—CN, —Y—NO₂, —Y—OH, —Y—OR^{a}, —Y—OC(═O)R^{a}, —Y—OC(═O)OR^{b}, —Y—OC(═O)NR^{c}R^{d}, —Y—SH, —Y—SR^{a}, —Y—S(═O)R^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{4b};
or two R^{4a} on the same atom are taken together to form an oxo;
each R^{4b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{4b} on the same atom are taken together to form an oxo;
Y is absent or C₁-C₆ alkylene optionally substituted with one or more deuterium, halogen, —CN, —OH, or —OR^{a};
Ring A is cycloalkyl, heterocycloalkyl, aryl, or heteroaryl;
L is absent or C₁-C₃ alkylene optionally substituted with one or more deuterium, halogen, —CN, —OH, or —OR^{a};
R^{6a} is —OH, —OR^{a}, or C₁-C₆alkyl;
R^{6b} is —OH, —OR^{a}, or C₁-C₆alkyl;
each R⁷ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R⁷ on the same atom are taken together to form an oxo;
n is 0-4;
each R is independently deuterium, halogen, —CN, —OH, —OR^{a}, —SH, —SR^{a}, —NR^{c}R^{d}, —NR^{c}C(═O)R^{b}, —C(═O)NR^{c}R^{d}, —C(═O)R^{a}, —C(═O)OR^{a}, C₁-C₆ alkyl, C₁-C₆ haloalkyl, C₁-C₆ hydroxyalkyl, C₁-C₆deuteroalkyl, C₁-C₆ aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl; m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or —F; or
X¹ and X² are taken together with the boron atom to which there are attached to form an optionally substituted cyclic boronate ester;
R^{X} is C₁-C₆ alkyl or cycloalkyl;
Z is hydrogen, R¹¹, —(R¹⁰)_{w}OR¹¹, —(R¹⁰)_{w}O(R¹⁰)_{w}OR¹¹, —R¹⁰OC(═O)R¹¹, —R¹⁰OC(═O)OR¹¹, —R¹⁰OC(═O)NHR¹¹, —R¹⁰OC(═O)N(R¹¹)₂, alkyloxyalkyl, acyloxyalkyl, or alkyl-[1,3]dioxol-2-one;
each R¹⁰ is independently —CH₂—, —CH(CH₃)—, —C(CH₃)₂—, or 1,1′-cyclopropylene;
each R¹¹ is independently C₁-C₆ alkyl, cycloalkyl, heterocycloalkyl, aryl, or heteroaryl; or
two R¹¹ are taken together with the nitrogen to which they are attached to form an optionally substituted heterocycloalkyl optionally substituted with C₁-C₆alkyl, C₁-C₆haloalkyl, C₁-C₆deuteroalkyl, C₁-C₆hydroxyalkyl, or C₁-C₆aminoalkyl;
w is 2-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
provided that the compound is not

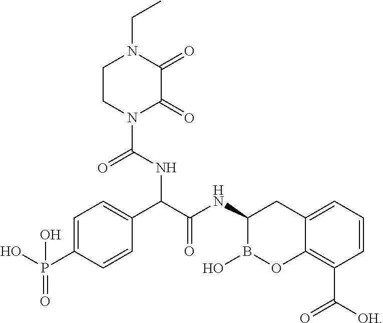 [SMILES: CCN1CCN(C(=O)NC(C(=O)N[C@H]2Cc3cccc(C(=O)O)c3OB2O)c2ccc(P(=O)(O)O)cc2)C(=O)C1=O]

Also provided herein are compounds of Formula (IIa) or (IIb), or a pharmaceutically acceptable salt, solvate, stereoisomer, tautomer, N-oxide, dimer, or trimer thereof:

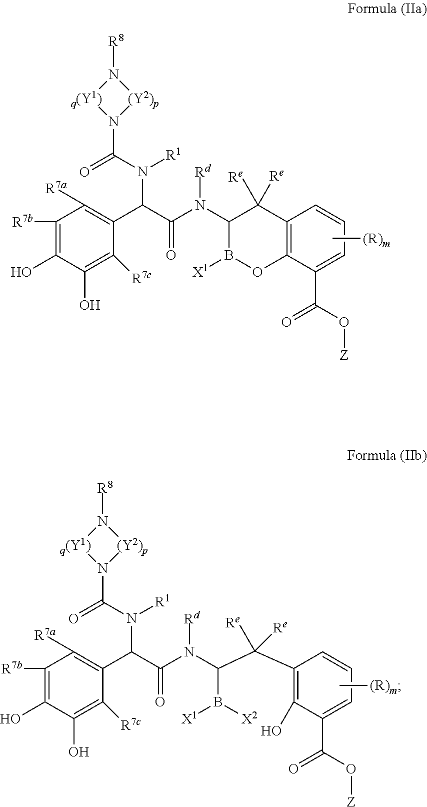

wherein:
R¹ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each Y¹ and Y² is independently —C(═O)— or —C(R^{Y})₂—;
each R^{Y} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
q is 1-3;
p is 1-3;
R⁸ is —(W)ᵤ-Ring B;
each W is independently —C(R^{W1})₂—, —O—, —NR^{W2}—, —S—, —S(═O)—, —S(═O)₂—, or —C(═O)—;
each R^{W1} is independently hydrogen, deuterium, halogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{W2} is independently hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or one R^{W1} and one R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{W1}, when present, are taken together to form a cycloalkyl or heterocycloalkyl; each optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
u is 1-10;
Ring B is cycloalkyl, heterocycloalkyl, aryl, or heteroaryl; each optionally substituted with one or more R^{B};
each R^{B} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R^{7a} is hydrogen, deuterium, halogen, —OH, —OR^{a}, —NR^{c}R^{d}, or C₁-C₆alkyl;
R^{7b} is deuterium, halogen, —OH, —OR^{a}, —NR^{c}R^{d}, or C₁-C₆alkyl;
R^{7c} is deuterium, halogen, —OH, —OR^{a}, —NR^{c}R^{d}, or C₁-C₆alkyl;
each R is independently deuterium, halogen, —CN, —OH, —OR^{a}, —SH, —SR^{a}, —NR^{c}R^{d}, —NR^{c}C(═O)R^{b}, —C(═O)NR^{c}R^{d}, —C(═O)R^{a}, —C(═O)OR^{a}, C₁-C₆ alkyl, C₁-C₆ haloalkyl, C₁-C₆deuteroalkyl, C₁-C₆ hydroxyalkyl, C₁-C₆ aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
m is 0-3;
R^{d} is hydrogen or C₁-C₆ alkyl;
each R^{c} is independently hydrogen, —CN, —OH, C₁-C₆ alkyl, or cycloalkyl;
X¹ and X² are independently —OH, —OR^{X}, or —F; or
X¹ and X² are taken together with the boron atom to which there are attached to form an optionally substituted cyclic boronate ester;
R^{X} is C₁-C₆ alkyl or cycloalkyl;
Z is hydrogen, R¹¹, —(R¹⁰)_{w}OR¹¹, —(R¹⁰)_{w}O(R¹⁰)_{w}OR¹¹, —R¹⁰OC(═O)R¹¹, —R¹⁰OC(═O)OR¹¹, —R¹⁰OC(═O)NHR¹¹, —R¹⁰OC(═O)N(R¹¹)₂, alkyloxyalkyl, acyloxyalkyl, or alkyl-[1,3]dioxol-2-one;
each R¹⁰ is independently —CH₂—, —CH(CH₃)—, —C(CH₃)₂—, or 1,1′-cyclopropylene;
each R¹¹ is independently C₁-C₆ alkyl, cycloalkyl, heterocycloalkyl, aryl, or heteroaryl; or
two R¹¹ are taken together with the nitrogen to which they are attached to form an optionally substituted heterocycloalkyl optionally substituted with C₁-C₆alkyl, C₁-C₆haloalkyl, C₁-C₆deuteroalkyl, C₁-C₆hydroxyalkyl, or C₁-C₆aminoalkyl;
w is 2-4;
Y is absent or C₁-C₆ alkylene optionally substituted with one or more deuterium, halogen, —CN, —OH, or —OR^{a};
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
provided that the compound is not

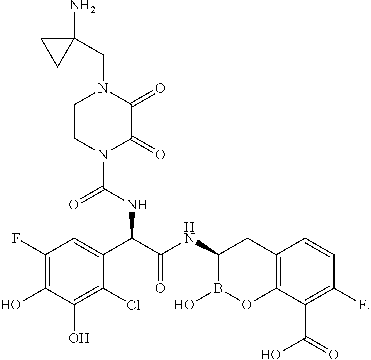 [SMILES: NC1(CN2CCN(C(=O)N[C@@H](C(=O)N[C@H]3Cc4ccc(F)c(C(=O)O)c4OB3O)c3cc(F)c(O)c(O)c3Cl)C(=O)C2=O)CC1]

Also provided is a pharmaceutical composition comprising a compound disclosed herein, or a pharmaceutically acceptable salt, solvate, stereoisomer, tautomer, N-oxide, dimer, or trimer thereof, and a pharmaceutically acceptable excipient.

Also provided is a method of treating a bacterial infection in a subject, comprising administering to the subject an effective amount of a compound disclosed herein, or a pharmaceutically acceptable salt, solvate, stereoisomer, tautomer, N-oxide, dimer, or trimer thereof, or a pharmaceutical composition disclosed herein. Also disclosed herein is a method of inhibiting a bacterial penicillin-binding protein in a human infected with a bacterial infection, comprising contacting said bacterial penicillin-binding protein with an effective amount of a compound disclosed herein, or a pharmaceutically acceptable salt, solvate, stereoisomer, tautomer, N-oxide, dimer, or trimer thereof, or a pharmaceutical composition disclosed herein. In some embodiments, the bacterial infection is caused by Neisseria gonorrhoeae. In some embodiments, the bacterial infection is caused by Burkholderia pseudomallei. In some embodiments, the bacterial infection is caused by Pseudomonas aeruginosa. In some embodiments, the bacterial infection is caused by Acinetobacter baumannii. In some embodiments, the bacterial infection is caused by Pseudomonas aeruginosa/Acinetobacter baumannii. In some embodiments, the bacterial infection is caused by a carbapenem-resistant enterobacterales (CRE).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Over the decades of clinical use of beta-lactam antibiotics, bacteria have evolved resistance mechanisms that compromise beta-lactam utility, including production of easily transferable, broad-spectrum beta-lactamases that are able to efficiently hydrolyze the beta lactam ring. These enzymes, now counting >1300 variants, have spread throughout Enterobacteriaceae. The rapid spread of this mechanism of bacterial resistance severely limits beta-lactam therapeutic options.

Novel non-beta-lactam compounds that inhibit the transpeptidase function of PBPs and are not degraded by beta-lactamases would represent a major advance in the treatment of resistant bacterial infections, essentially circumventing >70 years of bacterial evolution to protect the function of the penicillin-binding proteins in cell wall biosynthesis. The present invention is directed to certain boron-based compounds (boronic acids and cyclic boronic acid esters) which are PBP inhibitors and antibacterial compounds. The compounds and their pharmaceutically acceptable salts are useful for the treatment of bacterial infections, particularly antibiotic resistant bacterial infections. Some embodiments include compounds, compositions, pharmaceutical compositions, use, and preparation thereof.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The term “antibiotic” refers to a compound or composition which decreases the viability of a microorganism, or which inhibits the growth or proliferation of a microorganism. The phrase “inhibits the growth or proliferation” means increasing the generation time (i.e., the time required for the bacterial cell to divide or for the population to double) by at least about 2-fold. Preferred antibiotics are those which can increase the generation time by at least about 10-fold or more (e.g., at least about 100-fold or even indefinitely, as in total cell death). As used in this disclosure, an antibiotic is further intended to include an antimicrobial, bacteriostatic, or bactericidal agent. Examples of antibiotics suitable for use with respect to the present invention include penicillins, cephalosporins, and carbapenems.

The term “β-lactam antibiotic” refers to a compound with antibiotic properties that contains a β-lactam functionality. Non-limiting examples of β-lactam antibiotics useful with respect to the invention include penicillins, cephalosporins, penems, carbapenems, and monobactams.

The term “β-lactamase” denotes a protein capable of inactivating a β-lactam antibiotic. The β-lactamase can be an enzyme which catalyzes the hydrolysis of the β-lactam ring of a β-lactam antibiotic. Of particular interest herein are microbial β-lactamases. The β-lactamase may be, for example, a serine β-lactamase or a metallo-β-lactamase.

The term “penicillin-binding protein” (“PBP”) refers to a family of indispensable bacterial enzymes responsible for the synthesis of peptidoglycan, an essential structural polymer found exclusively in the cell wall of bacteria. This family of proteins encompasses three class. Class A are high molecular weight bifunctional enzymes possessing both glycosyltransferase (GTase) and transpeptidase (TPase) activities, while class B are monofunctional high molecular weight transpeptidases and class C are low molecular weight remodeling enzymes that include D,D-carboxypeptidases and D,D-endopeptidases. Penicillin binding proteins (PBPs) are the targets of β-lactam antibiotics, agents that covalently modify the active site of TPases and block the synthesis and remodeling of peptidoglycan, leading to rapid bacterial cell lysis of actively dividing cells.

“Amino” refers to the —NH₂ substituent.

“Oxo” refers to the ═O substituent.

“Oxime” refers to the ═N—OH substituent.

“Thioxo” refers to the ═S substituent.

“Alkyl” refers to a linear or branched hydrocarbon chain, which is fully saturated. Alkyl may have from one to thirty carbon atoms. An alkyl comprising up to 30 carbon atoms is referred to as a C₁-C₃₀ alkyl, likewise, for example, an alkyl comprising up to 12 carbon atoms is a C₁-C₁₂ alkyl. An alkyl comprising up to 6 carbons is a C₁-C₆ alkyl. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl, C₄-C₈ alkyl, and C₅-C₁₂ alkyl. In some embodiments, the alkyl group is C₁-C₆ alkyl. Representative alkyl groups include, but are not limited to, methyl, ethyl, n-propyl, 1-methylethyl (isopropyl), n-butyl, i-butyl, s-butyl, n-pentyl, 1,1-dimethylethyl (t-butyl), 2-ethylpropyl, and the like. Representative linear alkyl groups include, but are not limited to, methyl, ethyl, n-propyl, n-butyl, n-pentyl and the like. In some embodiments, the alkyl is substituted with an optionally substituted aryl to form an optionally substituted aralkyl. In some embodiments, the alkyl is substituted with an optionally substituted heteroaryl to form an optionally substituted heteroarylalkyl. In some embodiments, the alkyl is substituted with an optionally substituted cycloalkyl to form an optionally substituted cycloalkylalkyl. In some embodiments, the alkyl is substituted with an optionally substituted heterocycloalkyl to form an optionally substituted heterocycloalkylalkyl. In some embodiments, the alkyl group is optionally substituted with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to a straight or branched hydrocarbon chain, containing at least one carbon-carbon double bond. In certain embodiments, alkenyl comprises two to twelve (C₂-C₁₂ alkenyl) carbon atoms, or two to eight carbon atoms (C₂-C₈ alkenyl), or two to six carbon atoms (C₂-C₆ alkenyl) or two to four carbon atoms (C₂-C₄ alkenyl). The alkenyl may be attached to the rest of the molecule by a single bond, for example, ethenyl (i.e., vinyl), prop-1-enyl (i.e., allyl), but-1-enyl, pent-1-enyl, penta-1,4-dienyl, and the like. Alkenyl may be attached to the rest of the molecule by a double bond, e.g., ═CH₂, ═CH(CH₂)₃CH₃. In some embodiments, the alkenyl group is optionally substituted with oxo, halogen, amino, nitrile, nitro, hydroxyl, haloalkyl, alkoxy, aryl, cycloalkyl, heterocycloalkyl, heteroaryl, and the like. In some embodiments, the alkenyl is optionally substituted with oxo, halogen, —CN, —CF₃, —OH, —OMe, —NH₂, or —NO₂. In some embodiments, the alkenyl is optionally substituted with oxo, halogen, —CN, —CF₃, —OH, or —OMe. In some embodiments, the alkenyl is optionally substituted with halogen.

“Alkynyl” refers to a straight or branched hydrocarbon chain group, containing at least one carbon-carbon triple bond. In certain embodiments, alkynyl comprises two to twelve (C₂-C₁₂ alkynyl) carbon atoms, or two to eight carbon atoms (C₂-C₈ alkynyl), or two to six carbon atoms (C₂-C₆ alkynyl) or two to four carbon atoms (C₂-C₄ alkynyl). The alkynyl may be attached to the rest of the molecule by a single bond, for example, ethynyl, propynyl, butynyl, pentynyl, hexynyl, and the like. In some embodiments, the alkynyl group is optionally substituted with oxo, halogen, amino, nitrile, nitro, hydroxyl, haloalkyl, alkoxy, aryl, cycloalkyl, heterocycloalkyl, heteroaryl, and the like. In some embodiments, the alkynyl is optionally substituted with oxo, halogen, —CN, —CF₃, —OH, —OMe, —NH₂, or —NO₂. In some embodiments, the alkynyl is optionally substituted with oxo, halogen, —CN, —CF₃, —OH, or —OMe. In some embodiments, the alkynyl is optionally substituted with halogen.

“Alkylene” or “alkylene chain” refers to a straight or branched divalent hydrocarbon chain linking the rest of the molecule to a radical group, consisting solely of carbon and hydrogen, containing no unsaturation and having, for example, from one to twelve carbon atoms (C₁-C₁₂ alkylene), e.g., methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (C₁-C₈ alkylene), or one to five carbon atoms (C₁-C₅ alkylene), or one to four carbon atoms (C₁-C₄ alkylene), or one to three carbon atoms (C₁-C₃ alkylene), or one to two carbon atoms (C₁-C₂ alkylene). In other embodiments, an alkylene comprises one carbon atom (C₁ alkylene), or two carbon atoms (C₂ alkylene). In certain embodiments, an alkylene comprises two to five carbon atoms (e.g., C₂-C₅ alkylene). In some embodiments, the alkylene is optionally substituted with oxo, halogen, —CN, —CF₃, —OH, —OMe, —NH₂, or —NO₂. In some embodiments, the alkylene is optionally substituted with oxo, halogen, —CN, —CF₃, —OH, or —OMe. In some embodiments, the alkylene is optionally substituted with halogen.

“Alkoxy” refers to a radical of the formula —O-alkyl where alkyl is as defined herein. Unless stated otherwise specifically in the specification, an alkoxy group may be optionally substituted as described above for alkyl.

“Aryl” refers to an aromatic monocyclic hydrocarbon or aromatic multicyclic hydrocarbon ring system by removing a hydrogen atom from a ring carbon atom. Aryl may include cycles with six to eighteen carbon atoms, where at least one of the rings in the ring system is aromatic, i.e., it contains a cyclic, delocalized (4n+2) π-electron system in accordance with the Hückel theory. In some embodiments, the aryl is a monocyclic, bicyclic, tricyclic or tetracyclic ring system, which may include fused ring system (when fused with a cycloalkyl or heterocycloalkyl ring, the aryl is bonded through an aromatic ring atom). In some embodiments, the aryl is a 6 to 10-membered aryl. In some embodiments, the aryl is a 6-membered aryl. In some embodiments, the aryl is a 10-membered aryl. The ring system from which aryl groups are derived include, but are not limited to, groups such as benzene, fluorene, indane, indene, tetralin and naphthalene. In some embodiments, the aryl is optionally substituted with halogen, amino, nitrile, nitro, hydroxyl, alkyl, haloalkyl, alkoxy, aryl, aralkyl, cycloalkyl, heterocycloalkyl, heteroaryl, and the like. In some embodiments, the aryl is optionally substituted with halogen, —CN, -Me, -Et, —CF₃, —OH, —OMe, —NH₂, —NO₂, or cyclopropyl. In some embodiments, the aryl is optionally substituted with oxo, halogen, —CN, -Me, -Et, —CF₃, —OH, —OMe, or cyclopropyl. In some embodiments, the aryl is optionally substituted with halogen.

“Aryloxy” refers to a radical bonded through an oxygen atom of the formula —O-aryl, where aryl is as described above.

“Aralkyl” refers to a radical of the formula —R^{h}-aryl where R^{h} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Boronate ester” refers to —B(OR^{k})₂ wherein each R^{k} are independently hydrogen, optionally substituted alkyl, optionally substituted cycloalkyl, optionally substituted heterocycloalkyl, optionally substituted aryl, optionally substituted heteroaryl, (poly ethylene glycol) ethyl, or an optionally substituted saccharide provided that they are not both hydrogen. In some embodiments, each R^{k} is alkyl. In some embodiments, two R^{k} may be taken together with the atom to which they are attached to form an optionally substituted heterocycle or a cyclic boronate ester. In some embodiments, the cyclic boronate ester is formed from pinanediol, pinacol, 1,2-ethanediol, 1,3-propanediol, 1,2-propanediol, 2,3-butanediol, 1,2-diisopropylethandiol, 5,6-decanediol, 1,2-dicyclohexylethanediol, diethanolamine, 1,2-diphenyl-1,2-ethanediol, 2,6,6-trimethylbicyclo[3.1.1]heptane-2,3-diol, or (1S,2S,3R,5S)-2,6,6-trimethylbicyclo[3.1.1]heptane-2,3-diol.

“Cycloalkyl” refers to a saturated or partially unsaturated, monocyclic or polycyclic hydrocarbon. In certain embodiments, the cycloalkyl includes fused (when fused with an aryl or a heteroaryl ring, the cycloalkyl is bonded through a non-aromatic ring atom) or bridged ring systems. In certain embodiments, the cycloalkyl comprises from three to twenty carbon atoms (C₃-C₂₀ cycloalkyl), or three to ten carbon atoms (C₃-C₁₀ cycloalkyl), or three to eight carbon atoms (C₃-C₈ cycloalkyl), or three to six carbon atoms (C₃-C₆ cycloalkyl). In some embodiments, the cycloalkyl is a 3- to 6-membered cycloalkyl. In some embodiments, the cycloalkyl is a 3- to 8-membered cycloalkyl. Examples of monocyclic cycloalkyls include, e.g., cyclopropyl, cyclobutyl, cyclopentyl, cyclohexyl, cycloheptyl, and cyclooctyl. Polycyclic cycloalkyl include, for example, adamantyl, norbornyl (i.e., bicyclo[2.2.1]heptanyl), norbornenyl, decalinyl, 7,7-dimethyl-bicyclo[2.2.1]heptanyl, and the like. In some embodiments, the cycloalkyl is optionally substituted with oxo, halogen, amino, nitrile, nitro, hydroxyl, alkyl, haloalkyl, alkoxy, aryl, aralkyl, cycloalkyl, heterocycloalkyl, heteroaryl, and the like. In some embodiments, the cycloalkyl is optionally substituted with oxo, halogen, —CN, -Me, -Et, —CF₃, —OH, —OMe, —NH₂, —NO₂, or cyclopropyl. In some embodiments, the cycloalkyl is optionally substituted with oxo, halogen, —CN, -Me, -Et, —CF₃, —OH, —OMe, or cyclopropyl. In some embodiments, the cycloalkyl is optionally substituted with halogen.

“Cycloalkylalkyl” refers to a radical of the formula —R^{h}-cycloalkyl where R^{h} is an alkylene chain as defined above. The alkylene chain and the cycloalkyl radical are optionally substituted as described above.

“Halo” or “halogen” refers to bromo, chloro, fluoro, or iodo. In some embodiments, halogen refers to chloro or fluoro.

“Heterocycloalkyl” refers to a saturated or partially unsaturated ring that comprises two to twenty carbon atoms and at least one heteroatom. In certain embodiments, the heteroatoms are independently selected from N, O, Si, P, B, and S atoms. In certain embodiments, the heteroatoms are independently selected from N, O, and S atoms. The heterocycloalkyl may be selected from monocyclic or bicyclic, fused (when fused with an aryl or a heteroaryl ring, the heterocycloalkyl is bonded through a non-aromatic ring atom) or bridged ring systems. The heteroatoms in the heterocycloalkyl are optionally oxidized. One or more nitrogen atoms, if present, are optionally quaternized. The heterocycloalkyl is partially or fully saturated. The heterocycloalkyl is attached to the rest of the molecule through any atom of the heterocycloalkyl, valence permitting, such as any carbon or nitrogen atoms of the heterocycloalkyl. In certain embodiments, the heterocycloalkyl comprises from two to twenty carbon atoms (C₂-C₂₀ heterocycloalkyl), or two to ten carbon atoms (C₂-C₁₀ heterocycloalkyl), or two to eight carbon atoms (C₂-C₈ heterocycloalkyl), or two to six carbon atoms (C₂-C₆ heterocycloalkyl). In some embodiments, the heterocycloalkyl is a 3- to 6-membered heterocycloalkyl. In some embodiments, the heterocycloalkyl is a 3- to 8-membered heterocycloalkyl. In some embodiments, the heterocycloalkyl is a 5- to 6-membered heterocycloalkyl. In some embodiments, the heterocycloalkyl is a 5-membered heterocycloalkyl. In some embodiments, the heterocycloalkyl is a 6-membered heterocycloalkyl. Examples of heterocycloalkyl include, but are not limited to, azetidinyl, aziridyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocycloalkyl is optionally substituted with oxo, halogen, amino, nitrile, nitro, hydroxyl, alkyl, haloalkyl, alkoxy, aryl, aralkyl, cycloalkyl, heterocycloalkyl, heteroaryl, and the like. In some embodiments, the heterocycloalkyl is optionally substituted with oxo, halogen, —CN, -Me, -Et, —CF₃, —OH, —OMe, —NH₂, —NO₂, or cyclopropyl. In some embodiments, the heterocycloalkyl is optionally substituted with oxo, halogen, —CN, -Me, -Et, —CF₃, —OH, —OMe, or cyclopropyl. In some embodiments, the heterocycloalkyl is optionally substituted with halogen.

“Heterocycloalkylalkyl” refers to a radical of the formula —R^{h}-heterocycloalkyl where R^{h} is an alkylene chain as defined above. If the heterocycloalkyl is a nitrogen-containing heterocycloalkyl, the heterocycloalkyl is optionally attached to the alkyl radical at the nitrogen atom. The alkylene chain of the heterocycloalkylalkyl radical is optionally substituted as defined above for an alkylene chain. The heterocycloalkyl part of the heterocycloalkylalkyl radical is optionally substituted as defined above for a heterocycloalkyl group.

“Heteroaryl” refers to a 5- to 14-membered ring system comprising hydrogen atoms, one to thirteen carbon atoms, one to six heteroatoms selected from the group consisting of nitrogen, oxygen, phosphorous and sulfur, and at least one aromatic ring. In some embodiments, the heteroaryl is a 5- or 6-membered heteroaryl. In some embodiments, the heteroaryl is a 5-membered heteroaryl. In some embodiments, the heteroaryl is a 6-membered heteroaryl. In some embodiments, the heteroaryl is a monocyclic, bicyclic, tricyclic or tetracyclic ring system, which may include fused ring systems (when fused with a cycloalkyl or heterocycloalkyl ring, the heteroaryl is bonded through an aromatic ring atom); and the nitrogen, carbon or sulfur atoms in the heteroaryl may be optionally oxidized; the nitrogen atom may be optionally quaternized. In some embodiments, the heteroaryl is a 5- to 10-membered heteroaryl. In some embodiments, the heteroaryl is a 10-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). In some embodiments, the heteroaryl is optionally substituted with halogen, amino, nitrile, nitro, hydroxyl, alkyl, haloalkyl, alkoxy, aryl, aralkyl, cycloalkyl, heterocycloalkyl, heteroaryl, and the like. In some embodiments, the heteroaryl is optionally substituted with halogen, —CN, -Me, -Et, —CF₃, —OH, —OMe, —NH₂, —NO₂, or cyclopropyl. In some embodiments, the heteroaryl is optionally substituted with halogen, —CN, -Me, -Et, —CF₃, —OH, —OMe, or cyclopropyl. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In some embodiments, treatment also includes prophylactic treatment (e.g., administration of a composition described herein when an individual is suspected to be suffering from a bacterial infection).

### Compounds

Described herein are compounds that modulate the activity of penicillin-binding proteins. In some embodiments, the compounds described herein inhibit beta-lactamase. In certain embodiments, the compounds described herein are useful in the treatment of bacterial infections. In some embodiments, the bacterial infection is an upper or lower respiratory tract infection, a urinary tract infection, an intra-abdominal infection, or a skin infection. In some embodiments, the bacterial infection is uncomplicated or complicated urinary tract infections, uncomplicated or complicated gonorrhea, upper or lower respiratory tract infections, skin or skin structure infections, intra-abdominal infections, central nervous system infections, blood stream infections, or systemic infections.

Disclosed herein is a compound of Formula (Ia) or (Ib), or a pharmaceutically acceptable salt, solvate, stereoisomer, tautomer, N-oxide, dimer, or trimer thereof:

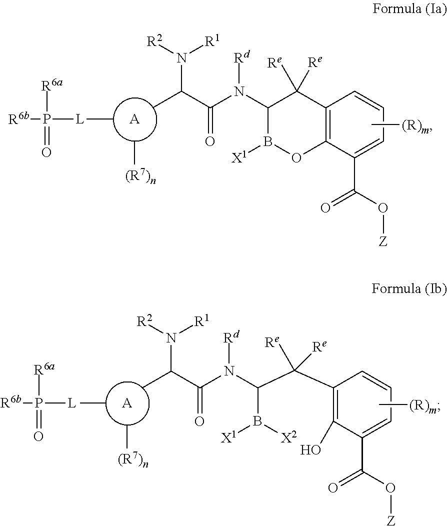

wherein:
R¹ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R² is hydrogen, C₁-C₆ alkyl, —C(═O)R³, —S(═O)₂R³, —C(═O)N(R⁴)₂, or —S(═O)₂N(R⁴)₂;
R³ is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{3a};
each R^{3a} is independently deuterium, halogen, —Y—CN, —Y—NO₂, —Y—OH, —Y—OR^{a}, —Y—OC(═O)R^{a}, —Y—OC(═O)OR^{b}, —Y—OC(═O)NR^{c}R^{d}, —Y—SH, —Y—SR^{a}, —Y—S(═O)R^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, —Y-cycloalkyl, —Y-heterocycloalkyl, —Y-aryl, or —Y-heteroaryl; wherein the alkyl, alkenyl, alkynyl, cycloalkyl, heterocycloalkyl, aryl, and heteroaryl is optionally substituted with one or more R^{3b};
or two R^{3a} on the same atom are taken together to form an oxo;
each R^{3b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{3b} on the same atom are taken together to form an oxo;
each R⁴ is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{4a};
each R^{4a} is independently deuterium, halogen, —Y—CN, —Y—NO₂, —Y—OH, —Y—OR^{a}, —Y—OC(═O)R^{a}, —Y—OC(═O)OR^{b}, —Y—OC(═O)NR^{c}R^{d}, —Y—SH, —Y—SR^{a}, —Y—S(═O)R^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{4b};
or two R^{4a} on the same atom are taken together to form an oxo;
each R^{4b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{4b} on the same atom are taken together to form an oxo;
Y is absent or C₁-C₆ alkylene optionally substituted with one or more deuterium, halogen, —CN, —OH, or —OR^{a};
Ring A is cycloalkyl, heterocycloalkyl, aryl, or heteroaryl;
L is absent or C₁-C₃ alkylene optionally substituted with one or more deuterium, halogen, —CN, —OH, or —OR^{a};
R^{6a} is —OH, —OR^{a}, or C₁-C₆alkyl;
R^{6b} is —OH, —OR^{a}, or C₁-C₆alkyl;
each R⁷ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R⁷ on the same atom are taken together to form an oxo;
n is 0-4;
each R is independently deuterium, halogen, —CN, —OH, —OR^{a}, —SH, —SR^{a}, —NR^{c}R^{d}, —NR^{c}C(═O)R^{b}, —C(═O)NR^{c}R^{d}, —C(═O)R^{a}, —C(═O)OR^{a}, C₁-C₆ alkyl, C₁-C₆ haloalkyl, C₁-C₆ hydroxyalkyl, C₁-C₆deuteroalkyl, C₁-C₆ aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or —F; or
X¹ and X² are taken together with the boron atom to which there are attached to form an optionally substituted cyclic boronate ester;
R^{X} is C₁-C₆ alkyl or cycloalkyl;
Z is hydrogen, R¹¹, —(R¹⁰)_{w}OR¹¹, —(R¹⁰)_{w}O(R¹⁰)_{w}OR¹¹, —R¹⁰OC(═O)R¹¹, —R¹⁰OC(═O)OR¹¹, —R¹⁰OC(═O)NHR¹¹, —R¹⁰OC(═O)N(R¹¹)₂, alkyloxyalkyl, acyloxyalkyl, or alkyl-[1,3]dioxol-2-one;
each R¹⁰ is independently —CH₂—, —CH(CH₃)—, —C(CH₃)₂—, or 1,1′-cyclopropylene;
each R¹¹ is independently C₁-C₆ alkyl, cycloalkyl, heterocycloalkyl, aryl, or heteroaryl; or
two R¹¹ are taken together with the nitrogen to which they are attached to form an optionally substituted heterocycloalkyl optionally substituted with C₁-C₆alkyl, C₁-C₆haloalkyl, C₁-C₆deuteroalkyl, C₁-C₆hydroxyalkyl, or C₁-C₆aminoalkyl;
w is 2-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
provided that the compound is not

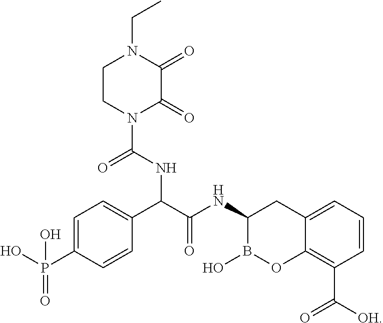 [SMILES: CCN1CCN(C(=O)NC(C(=O)N[C@H]2Cc3cccc(C(=O)O)c3OB2O)c2ccc(P(=O)(O)O)cc2)C(=O)C1=O]

In some embodiments of a compound of Formula (Ia) or (Ib), R² is C₁-C₆ alkyl. In some embodiments of a compound of Formula (Ia) or (Ib), R² is —C(═O)R³ or —S(═O)₂R³. In some embodiments of a compound of Formula (Ia) or (Ib), R² is —C(═O)R³. In some embodiments of a compound of Formula (Ia) or (Ib), R² is —S(═O)₂R³. In some embodiments of a compound of Formula (Ia) or (Ib), R² is —C(═O)N(R⁴)₂ or —S(═O)₂N(R⁴)₂.

In some embodiments of a compound of Formula (Ia) or (Ib), R³ is C₁-C₆alkyl, cycloalkyl, heterocycloalkyl, aryl, or heteroaryl; wherein the alkyl, cycloalkyl, heterocycloalkyl, aryl, and heteroaryl is optionally substituted with one or more R^{3a}.

In some embodiments of a compound of Formula (Ia) or (Ib), R³ is cycloalkyl, heterocycloalkyl, aryl, or heteroaryl; wherein the cycloalkyl, heterocycloalkyl, aryl, and heteroaryl is optionally substituted with one or more R^{3a}.

In some embodiments of a compound of Formula (Ia) or (Ib), R³ is heterocycloalkyl optionally substituted with one or more R^{3a}.

In some embodiments of a compound of Formula (Ia) or (Ib), each R^{3a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{3b}; or two R^{3a} on the same atom are taken together to form an oxo.

In some embodiments of a compound of Formula (Ia) or (Ib), each R^{3a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆haloalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{3b}; or two R^{3a} on the same atom are taken together to form an oxo.

In some embodiments of a compound of Formula (Ia) or (Ib), each R^{3a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—NR^{b}C(═O)R^{a}, —Y—NR^{b}S(═O)₂R^{a}, —Y—C(═O)R^{a}, C₁-C₆alkyl, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{3b}; or two R^{3a} on the same atom are taken together to form an oxo.

In some embodiments of a compound of Formula (Ia) or (Ib), each R^{3a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—NR^{b}C(═O)R^{a}, —Y—NR^{b}S(═O)₂R^{a}, —Y—C(═O)R^{a}, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{3b}; or two R^{3a} on the same atom are taken together to form an oxo.

In some embodiments of a compound of Formula (Ia) or (Ib), each R^{3a} is not C₁-C₆alkyl.

In some embodiments of a compound of Formula (Ia) or (Ib), each R^{3b} is independently deuterium, halogen, —CN, —OH, —OR^{a}, —S(═O)₂R^{a}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R^{3b} on the same atom are taken together to form an oxo.

In some embodiments of a compound of Formula (Ia) or (Ib), each R^{3b} is independently deuterium, halogen, —CN, —OH, —OR^{a}, —S(═O)₂R^{a}, —C(═O)R^{a}, C₁-C₆alkyl, or C₁-C₆haloalkyl.

In some embodiments of a compound of Formula (Ia) or (Ib), R³ is

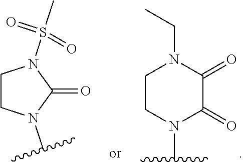 [SMILES: *N1CCN(S(C)(=O)=O)C1=O ; *N1CCN(CC)C(=O)C1=O]

In some embodiments of a compound of Formula (Ia) or (Ib), R³ is

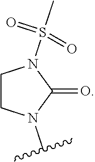 [SMILES: *N1CCN(S(C)(=O)=O)C1=O]

In some embodiments of a compound of Formula (Ia) or (Ib), R³ is

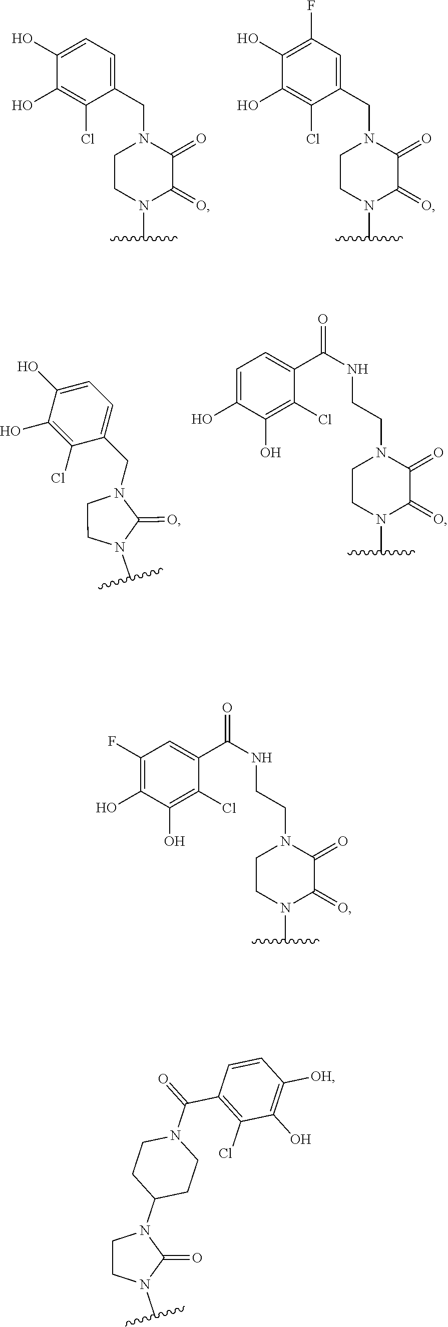 [SMILES: *N1CCN(Cc2ccc(O)c(O)c2Cl)C(=O)C1=O ; *N1CCN(Cc2cc(F)c(O)c(O)c2Cl)C(=O)C1=O ; *N1CCN(Cc2ccc(O)c(O)c2Cl)C1=O ; *N1CCN(CCNC(=O)c2ccc(O)c(O)c2Cl)C(=O)C1=O ; *N1CCN(CCNC(=O)c2cc(F)c(O)c(O)c2Cl)C(=O)C1=O ; *N1CCN(C2CCN(C(=O)c3ccc(O)c(O)c3Cl)CC2)C1=O]

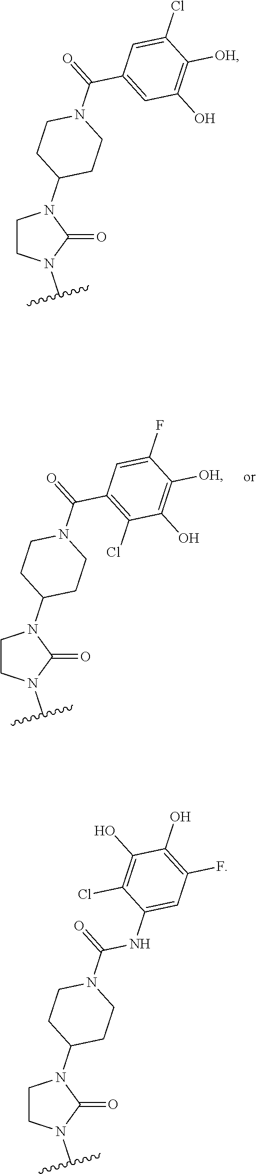 [SMILES: *N1CCN(C2CCN(C(=O)c3cc(O)c(O)c(Cl)c3)CC2)C1=O ; *N1CCN(C2CCN(C(=O)c3cc(F)c(O)c(O)c3Cl)CC2)C1=O ; *N1CCN(C2CCN(C(=O)Nc3cc(F)c(O)c(O)c3Cl)CC2)C1=O]

In some embodiments of a compound of Formula (Ia) or (Ib), R³ is

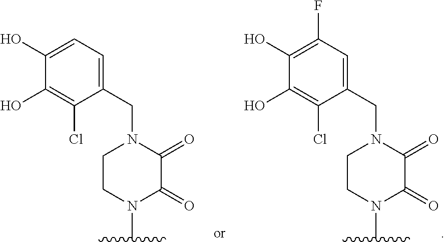 [SMILES: *N1CCN(Cc2ccc(O)c(O)c2Cl)C(=O)C1=O ; *N1CCN(Cc2cc(F)c(O)c(O)c2Cl)C(=O)C1=O]

In some embodiments of a compound of Formula (Ia) or (Ib), R³ is

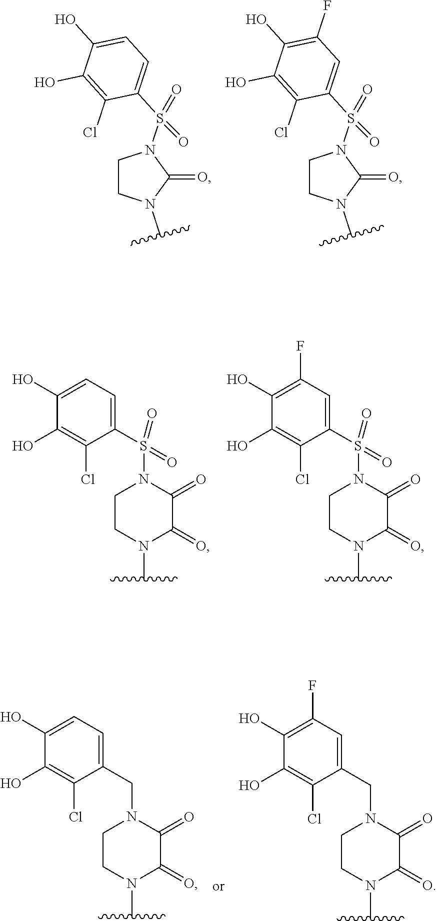 [SMILES: *N1CCN(S(=O)(=O)c2ccc(O)c(O)c2Cl)C1=O ; *N1CCN(S(=O)(=O)c2cc(F)c(O)c(O)c2Cl)C1=O ; *N1CCN(S(=O)(=O)c2ccc(O)c(O)c2Cl)C(=O)C1=O ; *N1CCN(S(=O)(=O)c2cc(F)c(O)c(O)c2Cl)C(=O)C1=O ; *N1CCN(Cc2ccc(O)c(O)c2Cl)C(=O)C1=O ; *N1CCN(Cc2cc(F)c(O)c(O)c2Cl)C(=O)C1=O]

In some embodiments of a compound of Formula (Ia) or (Ib), R³ is

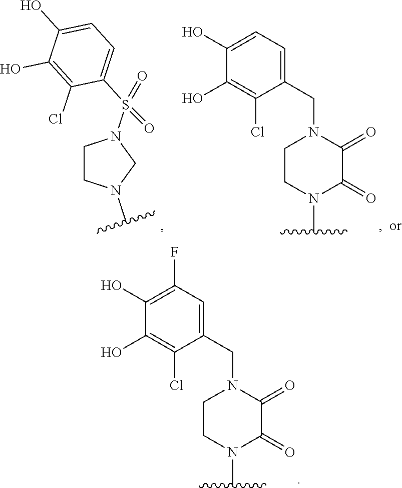 [SMILES: *N1CCN(S(=O)(=O)c2ccc(O)c(O)c2Cl)C1 ; *N1CCN(Cc2ccc(O)c(O)c2Cl)C(=O)C1=O ; *N1CCN(Cc2cc(F)c(O)c(O)c2Cl)C(=O)C1=O]

In some embodiments of a compound of Formula (Ia) or (Ib), each R⁴ is independently hydrogen, C₁-C₆alkyl, cycloalkyl, heterocycloalkyl, aryl, or heteroaryl; wherein the alkyl, cycloalkyl, heterocycloalkyl, aryl, and heteroaryl is optionally substituted with one or more R^{4a}.

In some embodiments of a compound of Formula (Ia) or (Ib), each R⁴ is independently hydrogen, cycloalkyl, heterocycloalkyl, aryl, or heteroaryl; wherein the cycloalkyl, heterocycloalkyl, aryl, and heteroaryl is optionally substituted with one or more R^{4a}.

In some embodiments of a compound of Formula (Ia) or (Ib), each R⁴ is independently hydrogen or heterocycloalkyl optionally substituted with one or more R^{4a}.

In some embodiments of a compound of Formula (Ia) or (Ib), each R^{4a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{4b}; or two R^{4a} on the same atom are taken together to form an oxo.

In some embodiments of a compound of Formula (Ia) or (Ib), each R^{4a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—NR^{b}C(═O)R^{a}, —Y—NR^{b}S(═O)₂R^{a}, —Y—C(═O)R^{a}, C₁-C₆alkyl, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{4b}; or two R^{4a} on the same atom are taken together to form an oxo.

In some embodiments of a compound of Formula (Ia) or (Ib), each R^{4b} is independently deuterium, halogen, —CN, —OH, —OR^{a}, —S(═O)₂R^{a}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R^{4b} on the same atom are taken together to form an oxo.

In some embodiments of a compound of Formula (Ia) or (Ib), each R^{4b} is independently deuterium, halogen, —CN, —OH, —OR^{a}, —S(═O)₂R^{a}, —C(═O)R^{a}, C₁-C₆alkyl, or C₁-C₆haloalkyl.

In some embodiments of a compound of Formula (Ia) or (Ib), the compound is of Formula (Ia-1) or Formula (Ib-1):

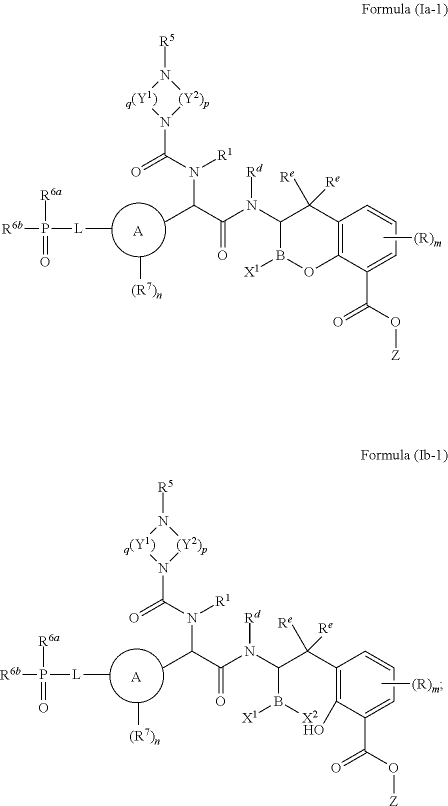

wherein:
each Y¹ and Y² is independently —C(═O)— or —C(R^{Y})₂—;
each R^{Y} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
q is 1-3;
p is 1-3;
R⁵ is —Y—OC(═O)R^{a}, —Y—OC(═O)OR^{b}, —Y—OC(═O)NR^{c}R^{d}, —Y—SH, —Y—SR^{a}, —Y—S(═O)R^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, —Y-cycloalkyl, —Y-heterocycloalkyl, —Y-aryl, or —Y-heteroaryl; wherein the alkyl, alkenyl, alkynyl, cycloalkyl, heterocycloalkyl, aryl, and heteroaryl is optionally substituted with one or more R^{5a}; and
each R^{5a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{5a} on the same atom are taken together to form an oxo.

In some embodiments of a compound of Formula (Ia-1) or (Ib-1), q is 1 or 2. In some embodiments of a compound of Formula (Ia-1) or (Ib-1), q is 1. In some embodiments of a compound of Formula (Ia-1) or (Ib-1), q is 2. In some embodiments of a compound of Formula (Ia-1) or (Ib-1), q is 3.

In some embodiments of a compound of Formula (Ia-1) or (Ib-1), p is 1 or 2. In some embodiments of a compound of Formula (Ia-1) or (Ib-1), p is 1. In some embodiments of a compound of Formula (Ia-1) or (Ib-1), p is 2. In some embodiments of a compound of Formula (Ia-1) or (Ib-1), p is 3.

In some embodiments of a compound of Formula (Ia-1) or (Ib-1), q is 2; each Y¹ is —C(R^{Y})₂—; p is 2; and each Y² is —C(═O)—. In some embodiments of a compound of Formula (Ia-1) or (Ib-1), q is 2; each Y¹ is —C(R^{Y})₂—; p is 1; and Y² is —C(═O)—. In some embodiments of a compound of Formula (Ia-1) or (Ib-1), q is 2; each Y¹ is —C(R^{Y})₂—; p is 2; and one Y² is —C(R^{Y})₂— and one Y² is —C(═O)—. In some embodiments of a compound of Formula (Ia-1) or (Ib-1), q is 2; one Y¹ is —C(R⁵)₂— and one Y¹ is —C(═O)—; p is 2; and one Y² is —C(R⁵)₂— and one Y² is —C(═O)—.

In some embodiments of a compound of Formula (Ia-1) or (Ib-1), each R^{Y} is independently hydrogen, halogen, or C₁-C₆alkyl.

In some embodiments of a compound of Formula (Ia-1) or (Ib-1), each R^{Y} is hydrogen.

In some embodiments of a compound of Formula (Ia-1) or (Ib-1),

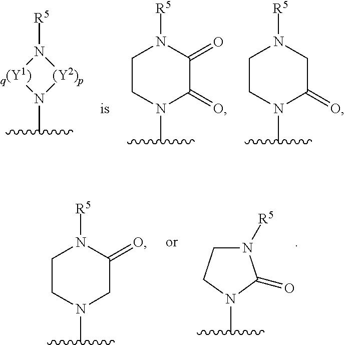

In some embodiments of a compound of Formula (Ia-1) or (Ib-1),

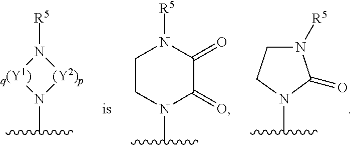

In some embodiments of a compound of Formula (Ia-1) or (Ib-1),

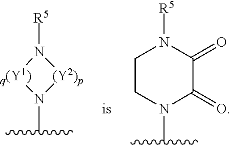

In some embodiments of a compound of Formula (Ia-1) or (Ib-1),

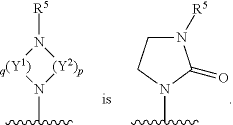

In some embodiments of a compound of Formula (Ia-1) or (Ib-1), R⁵ is —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{5a}.

In some embodiments of a compound of Formula (Ia-1) or (Ib-1), R⁵ is —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆haloalkyl, C₁-C₆deuteroalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{5a}.

In some embodiments of a compound of Formula (Ia-1) or (Ib-1), R⁵ is —Y—S(═O)₂R^{a}, —Y—S(═O)₂NR^{c}R^{d}, —Y—NR^{c}R^{d}, —Y—NR^{b}C(═O)R^{a}, —Y—NR^{b}S(═O)₂R^{a}, C₁-C₆alkyl, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{5a}.

In some embodiments of a compound of Formula (Ia-1) or (Ib-1), R⁵ is —Y—S(═O)₂R^{a}, —Y—S(═O)₂NR^{c}R^{d}, —Y—NR^{c}R^{d}, —Y—NR^{b}C(═O)R^{a}, —Y—NR^{b}S(═O)₂R^{a}, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{5a}.

In some embodiments of a compound of Formula (Ia-1) or (Ib-1), R⁵ is —Y—S(═O)₂R^{a}, —Y—NR^{b}C(═O)R^{a}, —Y-heterocycloalkyl, or —Y-aryl; wherein the heterocycloalkyl and aryl is optionally substituted with one or more R^{5a}.

In some embodiments of a compound of Formula (Ia-1) or (Ib-1), R⁵ is —Y—S(═O)₂R^{a}.

In some embodiments of a compound of Formula (Ia-1) or (Ib-1), R⁵ is —Y—NR^{b}C(═O)R^{a}.

In some embodiments of a compound of Formula (Ia-1) or (Ib-1), —Y-aryl; wherein the aryl is optionally substituted with one or more R^{5a}.

In some embodiments of a compound of Formula (Ia-1) or (Ib-1), R⁵ is —Y-heterocycloalkyl; wherein the heterocycloalkyl is optionally substituted with one or more R^{5a}.

In some embodiments of a compound of Formula (Ia-1) or (Ib-1), each R is not C₁-C₆alkyl.

In some embodiments of a compound of Formula (Ia-1) or (Ib-1), each R^{5a} is independently deuterium, halogen, —CN, —OH, —OR^{a}, —S(═O)₂R^{a}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R^{3b} on the same atom are taken together to form an oxo.

In some embodiments of a compound of Formula (Ia-1) or (Ib-1), each R^{5a} is independently deuterium, halogen, —CN, —OH, —OR^{a}, —S(═O)₂R^{a}, —C(═O)R^{a}, C₁-C₆alkyl, or C₁-C₆haloalkyl.

In some embodiments of a compound of Formula (Ia-1) or (Ib-1), R is methyl, ethyl, or

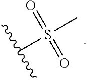 [SMILES: *S(C)(=O)=O]

In some embodiments of a compound of Formula (Ia-1) or (Ib-1), R⁵ is

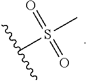 [SMILES: *S(C)(=O)=O]

In some embodiments of a compound of Formula (Ia-1) or (Ib-1), R⁵ is

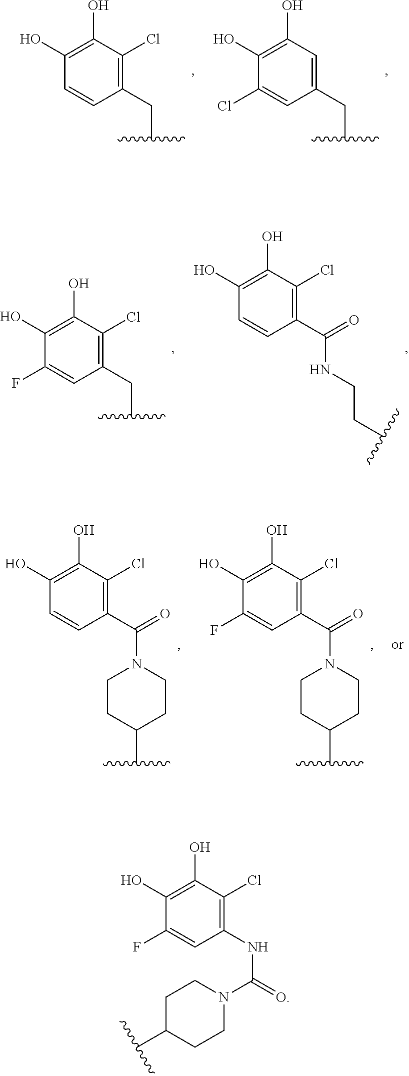

In some embodiments of a compound of Formula (Ia-1) or (Ib-1), R⁵ is

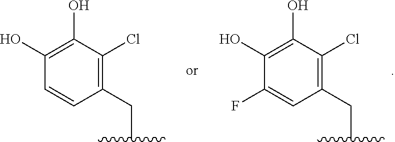 [SMILES: *Cc1ccc(O)c(O)c1Cl ; *Cc1cc(F)c(O)c(O)c1Cl]

In some embodiments of a compound of Formula (Ia-1) or (Ib-1), R⁵ is

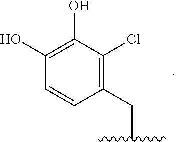 [SMILES: *Cc1ccc(O)c(O)c1Cl]

In some embodiments of a compound of Formula (Ia-1) or (Ib-1), R⁵ is

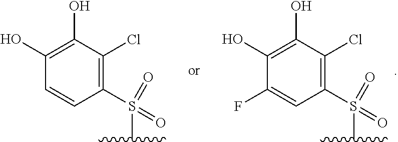 [SMILES: *S(=O)(=O)c1ccc(O)c(O)c1Cl ; *S(=O)(=O)c1cc(F)c(O)c(O)c1Cl]

In some embodiments of a compound of Formula (Ia), (Ia-1), (Ib), or (Ib-1), Ring A is aryl or heteroaryl. In some embodiments of a compound of Formula (Ia), (Ia-1), (Ib), or (Ib-1), Ring A is aryl. In some embodiments of a compound of Formula (Ia), (Ia-1), (Ib), or (Ib-1), Ring A is phenyl.

In some embodiments of a compound of Formula (Ia), (Ia-1), (Ib), or (Ib-1), Ring A is heteroaryl. In some embodiments of a compound of Formula (Ia), (Ia-1), (Ib), or (Ib-1), Ring A is a 5- or 6-membered heteroaryl. In some embodiments of a compound of Formula (Ia), (Ia-1), (Ib), or (Ib-1), Ring A is a 6-membered heteroaryl.

In some embodiments of a compound of Formula (Ia), (Ia-1), (Ib), or (Ib-1), L is absent. In some embodiments of a compound of Formula (Ia), (Ia-1), (Ib), or (Ib-1), L is C₁-C₃ alkylene optionally substituted with one or more deuterium, halogen, —CN, —OH, or —OR^{a}. In some embodiments of a compound of Formula (Ia), (Ia-1), (Ib), or (Ib-1), L is C₁ alkylene substituted with one or more halogen.

In some embodiments of a compound of Formula (Ia), (Ia-1), (Ib), or (Ib-1), R^{6a} is —OH or C₁-C₆alkyl. In some embodiments of a compound of Formula (Ia), (Ia-1), (Ib), or (Ib-1), R^{6a} is —OH.

In some embodiments of a compound of Formula (Ia), (Ia-1), (Ib), or (Ib-1), R^{6b} is —OH or C₁-C₆alkyl. In some embodiments of a compound of Formula (Ia), (Ia-1), (Ib), or (Ib-1), R^{6b} is —OH.

In some embodiments of a compound of Formula (Ia), (Ia-1), (Ib), or (Ib-1), each R⁷ is independently deuterium, halogen, —CN, —OH, —OR^{a}, —NR^{c}R^{d}, C₁-C₆alkyl, or C₁-C₆haloalkyl. In some embodiments of a compound of Formula (Ia), (Ia-1), (Ib), or (Ib-1), each R⁷ is independently halogen or —OH. In some embodiments of a compound of Formula (Ia), (Ia-1), (Ib), or (Ib-1), each R⁷ is independently halogen.

In some embodiments of a compound of Formula (Ia), (Ia-1), (Ib), or (Ib-1), n is 1 or 2. In some embodiments of a compound of Formula (Ia), (Ia-1), (Ib), or (Ib-1), n is 0 or 1. In some embodiments of a compound of Formula (Ia), (Ia-1), (Ib), or (Ib-1), n is 0. In some embodiments of a compound of Formula (Ia), (Ia-1), (Ib), or (Ib-1), n is 1. In some embodiments of a compound of Formula (Ia), (Ia-1), (Ib), or (Ib-1), n is 2. In some embodiments of a compound of Formula (Ia), (Ia-1), (Ib), or (Ib-1), n is 3.

Also disclosed herein is a compound of Formula (IIa) or (IIb), or a pharmaceutically acceptable salt, solvate, stereoisomer, tautomer, N-oxide, dimer, or trimer thereof:

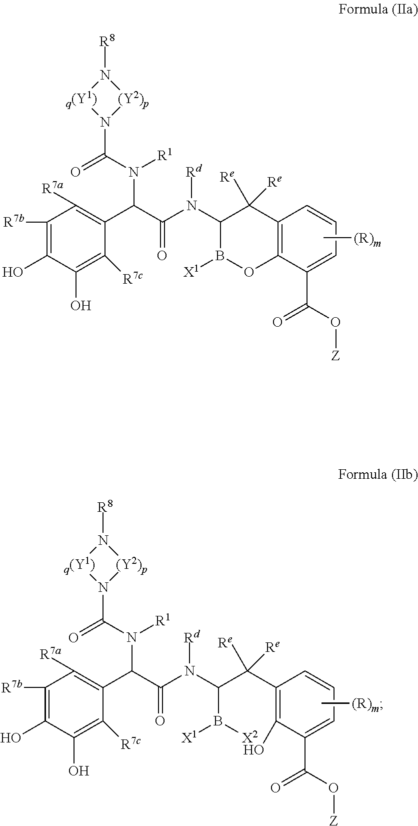

wherein:
R¹ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each Y¹ and Y² is independently —C(═O)— or —C(R^{Y})₂—;
each R^{Y} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
q is 1-3;
p is 1-3;
R⁸ is —(W)ᵤ-Ring B;
each W is independently —C(R^{W1})₂—, —O—, —NR^{W2}—, —S—, —S(═O)—, —S(═O)₂—, or —C(═O)—; each R^{W1} is independently hydrogen, deuterium, halogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{W2} is independently hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or one R^{W1} and one R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{W1}, when present, are taken together to form a cycloalkyl or heterocycloalkyl; each optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
u is 1-10;
Ring B is cycloalkyl, heterocycloalkyl, aryl, or heteroaryl; each optionally substituted with one or more R^{B};
each R^{B} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R^{7a} is hydrogen, deuterium, halogen, —OH, —OR^{a}, —NR^{c}R^{d}, or C₁-C₆alkyl;
R^{7b} is deuterium, halogen, —OH, —OR^{a}, —NR^{c}R^{d}, or C₁-C₆alkyl;
R^{7c} is deuterium, halogen, —OH, —OR^{a}, —NR^{c}R^{d}, or C₁-C₆alkyl; each R is independently deuterium, halogen, —CN, —OH, —OR^{a}, —SH, —SR^{a}, —NR^{c}R^{d}, —NR^{c}C(═O)R^{b}, —C(═O)NR^{c}R^{d}, —C(═O)R^{a}, —C(═O)OR^{a}, C₁-C₆ alkyl, C₁-C₆ haloalkyl, C₁-C₆deuteroalkyl, C₁-C₆ hydroxyalkyl, C₁-C₆ aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or —F; or
X¹ and X² are taken together with the boron atom to which there are attached to form an optionally substituted cyclic boronate ester;
R^{X} is C₁-C₆ alkyl or cycloalkyl;
Z is hydrogen, R¹¹, —(R¹⁰)_{w}OR¹¹, —(R¹⁰)_{w}O(R¹⁰)_{w}OR¹¹, —R¹⁰OC(═O)R¹¹, —R¹⁰OC(═O)OR¹¹, —R¹⁰OC(═O)NHR¹¹, —R¹⁰OC(═O)N(R¹¹)₂, alkyloxyalkyl, acyloxyalkyl, or alkyl-[1,3]dioxol-2-one;
each R¹⁰ is independently —CH₂—, —CH(CH₃)—, —C(CH₃)₂—, or 1,1′-cyclopropylene;
each R¹¹ is independently C₁-C₆ alkyl, cycloalkyl, heterocycloalkyl, aryl, or heteroaryl; or
two R¹¹ are taken together with the nitrogen to which they are attached to form an optionally substituted heterocycloalkyl optionally substituted with C₁-C₆alkyl, C₁-C₆haloalkyl, C₁-C₆deuteroalkyl, C₁-C₆hydroxyalkyl, or C₁-C₆aminoalkyl;
w is 2-4;
Y is absent or C₁-C₆ alkylene optionally substituted with one or more deuterium, halogen, —CN, —OH, or —OR^{a};
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
provided that the compound is not

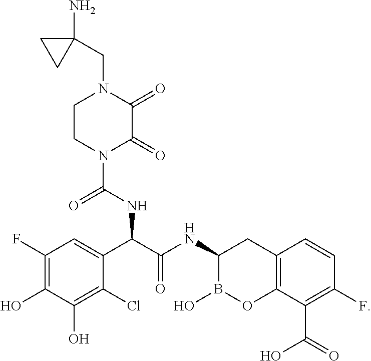 [SMILES: NC1(CN2CCN(C(=O)N[C@@H](C(=O)N[C@H]3Cc4ccc(F)c(C(=O)O)c4OB3O)c3cc(F)c(O)c(O)c3Cl)C(=O)C2=O)CC1]

In some embodiments of a compound of Formula (IIa) or (IIb), q is 1 or 2. In some embodiments of a compound of Formula (IIa) or (IIb), q is 1. In some embodiments of a compound of Formula (IIa) or (IIb), q is 2. In some embodiments of a compound of Formula (IIa) or (IIb), q is 3.

In some embodiments of a compound of Formula (IIa) or (IIb), p is 1 or 2. In some embodiments of a compound of Formula (IIa) or (IIb), p is 1. In some embodiments of a compound of Formula (IIa) or (IIb), p is 2. In some embodiments of a compound of Formula (IIa) or (IIb), p is 3.

In some embodiments of a compound of Formula (IIa) or (IIb), q is 2; each Y¹ is —C(R^{Y})₂—; p is 2; and each Y² is —C(═O)—. In some embodiments of a compound of Formula (IIa) or (IIb), q is 2; each Y¹ is —C(R^{Y})₂—; p is 1; and Y² is —C(═O)—. In some embodiments of a compound of Formula (IIa) or (IIb), q is 2; each Y¹ is —C(R^{Y})₂—; p is 2; and one Y² is —C(R^{Y})₂— and one Y² is —C(═O)—. In some embodiments of a compound of Formula (IIa) or (IIb), q is 2; one Y¹ is —C(R⁵)₂— and one Y¹ is —C(═O)—; p is 2; and one Y² is —C(R⁵)₂— and one Y² is —C(═O)—.

In some embodiments of a compound of Formula (IIa) or (IIb), each R^{Y} is independently hydrogen, halogen, or C₁-C₆alkyl. In some embodiments of a compound of Formula (IIa) or (IIb), each R^{Y} is hydrogen.

In some embodiments of a compound of Formula (IIa) or (IIb), is

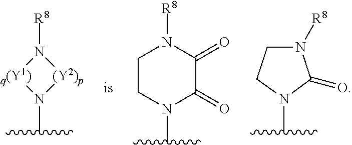

In some embodiments of a compound of Formula (IIa) or (IIb),

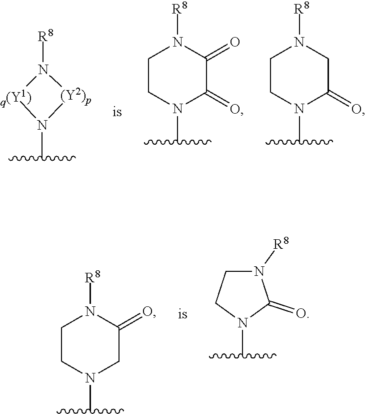

In some embodiments of a compound of Formula (IIa) or (IIb),

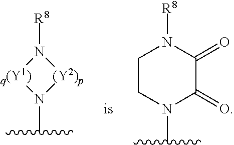

In some embodiments of a compound of Formula (IIa) or (IIb),

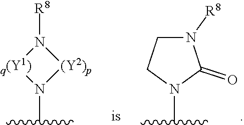

In some embodiments of a compound of Formula (IIa) or (IIb), R^{7a} is hydrogen or halogen. In some embodiments of a compound of Formula (IIa) or (IIb), R^{7a} is hydrogen.

In some embodiments of a compound of Formula (IIa) or (Ib), R^{7b} is halogen.

In some embodiments of a compound of Formula (IIa) or (IIb), R^{7c} is halogen.

In some embodiments of a compound of Formula (IIa) or (IIb), u is 1-8. In some embodiments of a compound of Formula (IIa) or (IIb), u is 1-7. In some embodiments of a compound of Formula (IIa) or (IIb), u is 1-6. In some embodiments of a compound of Formula (IIa) or (IIb), u is 1-5. In some embodiments of a compound of Formula (IIa) or (IIb), u is 3-8. In some embodiments of a compound of Formula (IIa) or (IIb), u is 4-8. In some embodiments of a compound of Formula (IIa) or (IIb), u is 5-8. In some embodiments of a compound of Formula (IIa) or (IIb), u is 4-6. In some embodiments of a compound of Formula (IIa) or (IIb), u is 2-6. In some embodiments of a compound of Formula (IIa) or (IIb), u is 2-5. In some embodiments of a compound of Formula (IIa) or (IIb), u is 2-4. In some embodiments of a compound of Formula (IIa) or (IIb), u is 1. In some embodiments of a compound of Formula (IIa) or (IIb), u is 2. In some embodiments of a compound of Formula (IIa) or (IIb), u is 3. In some embodiments of a compound of Formula (IIa) or (IIb), u is 4. In some embodiments of a compound of Formula (IIa) or (IIb), u is 5. In some embodiments of a compound of Formula (IIa) or (IIb), u is 6. In some embodiments of a compound of Formula (IIa) or (IIb), u is 7. In some embodiments of a compound of Formula (IIa) or (IIb), u is 8. In some embodiments of a compound of Formula (IIa) or (IIb), u is 9. In some embodiments of a compound of Formula (IIa) or (IIb), u is 10.

In some embodiments of a compound of Formula (IIa) or (IIb), each W is independently —C(R^{W1})₂—, —NR^{W2}—, or —C(═O)—. In some embodiments of a compound of Formula (IIa) or (IIb), each W is independently —C(R^{W1})₂—, —NR^{W2}—, or —S(═O)₂—. In some embodiments of a compound of Formula (IIa) or (IIb), each W is independently —C(R^{W1})₂—, —NR^{W2}—, —S(═O)₂—, or —C(═O)—. In some embodiments of a compound of Formula (IIa) or (IIb), each W is independently —C(R^{W1})₂— or —NR^{W2}—. In some embodiments of a compound of Formula (IIa) or (IIb), each W is independently —C(R^{W1})₂—.

In some embodiments of a compound of Formula (IIa) or (IIb), —(W)ᵤ— is —[C(R^{W1})₂]₁₋₄—NR^{W2}—C(═O)—, —[C(R^{W1})₂]₁₋₄—NR^{W2}—C(═O)—[C(R^{W1})₂]₁₋₂—, —[C(R^{W1})₂]₁₋₄—NR^{W2}—S(═O)₂—, —[C(R^{W1})₂]₁₋₄—NR^{W2}—C(═O)—NR^{W2}—, —[C(R^{W1})₂]₁₋₃—NR^{W2}—[C(R^{W1})₂]₁₋₃—, —[C(R^{W1})₂]₁₋₄—, —S(═O)₂—[C(R^{W1})₂]₁₋₄—NR^{W2}—C(═O)—, or —S(═O)₂—[C(R^{W1})₂]₁₋₄—NR^{W2}—C(═O)—NR^{W2}—. In some embodiments of a compound of Formula (IIa) or (IIb), —(W)ᵤ— is —[C(R^{W1})₂]₁₋₄—NR^{W2}—C(═O)— or —[C(R^{W1})₂]₁₋₄—NR^{W2}—C(═O)—[C(R^{W1})₂]₁₋₂—. In some embodiments of a compound of Formula (IIa) or (IIb), —(W)ᵤ— is —[C(R^{W1})₂]₁₋₄—NR^{W2}—C(═O)— or —[C(R^{W1})₂]₁₋₄—NR^{W2}—S(═O)₂—. In some embodiments of a compound of Formula (IIa) or (IIb), —(W)ᵤ— is —[C(R^{W1})₂]₁₋₄—NR^{W2}—C(═O)—, —[C(R^{W1})₂]₁₋₄—NR^{W2}—C(═O)—[C(R^{W1})₂]₁₋₂, —[C(R^{W1})₂]₁₋₄—NR^{W2}—S(═O)₂—, or —[C(R^{W1})₂]₁₋₄—NR^{W2}—C(═O)—NR^{W2}—. In some embodiments of a compound of Formula (IIa) or (IIb), —(W)ᵤ— is —[C(R^{W1})₂]₁₋₄—NR^{W2}—C(═O)—. In some embodiments of a compound of Formula (IIa) or (IIb), —(W)ᵤ— is —[C(R^{W1})₂]₁₋₄—NR^{W2}—C(═O)—[C(R^{W1})₂]₁₋₂—. In some embodiments of a compound of Formula (IIa) or (IIb), —(W)ᵤ— is —[C(R^{W1})₂]₁₋₄—NR^{W2}—S(═O)₂—[C(R^{W1})₂]₁₋₂—. In some embodiments of a compound of Formula (IIa) or (IIb), —(W)ᵤ— is —[C(R^{W1})₂]₁₋₄—NR^{W2}—S(═O)₂—. In some embodiments of a compound of Formula (IIa) or (IIb), —(W)ᵤ— is —[C(R^{W1})₂]₁₋₄—NR^{W2}—C(═O)—NR^{W2}—. In some embodiments of a compound of Formula (IIa) or (IIb), —(W)ᵤ— is —[C(R^{W1})₂]₁₋₃—NR^{W2}—[C(R^{W1})₂]₁₋₃—. In some embodiments of a compound of Formula (IIa) or (IIb), —(W)ᵤ— is —[C(R^{W1})₂]₁₋₄—. In some embodiments of a compound of Formula (IIa) or (IIb), —(W)ᵤ— is —S(═O)₂—[C(R^{W1})₂]₁₋₄—NR^{W2}—C(═O)—. In some embodiments of a compound of Formula (IIa) or (IIb), —(W)ᵤ— is —S(═O)₂—[C(R^{W1})₂]₁₋₄—NR^{W2}—C(═O)—NR^{W2}—.

In some embodiments of a compound of Formula (IIa) or (IIb), each R^{W1} is independently hydrogen, deuterium, halogen, C₁-C₆alkyl, or C₁-C₆haloalkyl. In some embodiments of a compound of Formula (IIa) or (IIb), each R^{W1} is independently hydrogen or C₁-C₆alkyl. In some embodiments of a compound of Formula (IIa) or (IIb), each R^{W1} is hydrogen.

In some embodiments of a compound of Formula (IIa) or (IIb), each R^{W2} is independently hydrogen, C₁-C₆alkyl, or C₁-C₆haloalkyl. In some embodiments of a compound of Formula (IIa) or (IIb), each R^{W2} is independently hydrogen or C₁-C₆alkyl. In some embodiments of a compound of Formula (IIa) or (IIb), each R^{W2} is hydrogen. In some embodiments of a compound of Formula (IIa) or (IIb), each R^{W2} is independently C₁-C₆alkyl.

In some embodiments of a compound of Formula (IIa) or (IIb), one R^{W1} and one R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (IIa) or (IIb), one R^{W1} and one R^{W2}, when present, are taken together to form a piperidinyl or pyrrolidinyl; each optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (IIa) or (IIb), one R^{W1} and one R^{W2}, when present, are taken together to form a piperidinyl or pyrrolidinyl. In some embodiments of a compound of Formula (IIa) or (IIb), one R^{W1} and one R^{W2}, when present, are taken together to form a piperidinyl.

In some embodiments of a compound of Formula (IIa) or (IIb), two R^{W1}, when present, are taken together to form a cycloalkyl or heterocycloalkyl; each optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

In some embodiments of a compound of Formula (IIa) or (IIb), two R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

In some embodiments of a compound of Formula (IIa) or (IIb), Ring B is heterocycloalkyl, aryl, or heteroaryl; each optionally substituted with one or more R^{B}.

In some embodiments of a compound of Formula (IIa) or (IIb), Ring B is aryl or heteroaryl; each optionally substituted with one or more R^{B}. In some embodiments of a compound of Formula (IIa) or (IIb), Ring B is aryl optionally substituted with one or more R^{B}. In some embodiments of a compound of Formula (IIa) or (IIb), Ring B is phenyl optionally substituted with one or more R^{B}. In some embodiments of a compound of Formula (IIa) or (IIb), Ring B is heteroaryl optionally substituted with one or more R^{B}. In some embodiments of a compound of Formula (IIa) or (IIb), Ring B is a 5- or 6-membered heteroaryl optionally substituted with one or more R^{B}.

In some embodiments of a compound of Formula (IIa) or (IIb), each R^{B} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (IIa) or (IIb), each R^{B} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl. In some embodiments of a compound of Formula (IIa) or (IIb), each R^{B} is independently halogen, —CN, —OH, —OR^{a}, —NR^{c}R^{d}, C₁-C₆alkyl, or C₁-C₆haloalkyl. In some embodiments of a compound of Formula (IIa) or (IIb), each R^{B} is independently halogen or OH.

In some embodiments of a compound of Formula (IIa) or (IIb), R⁸ is

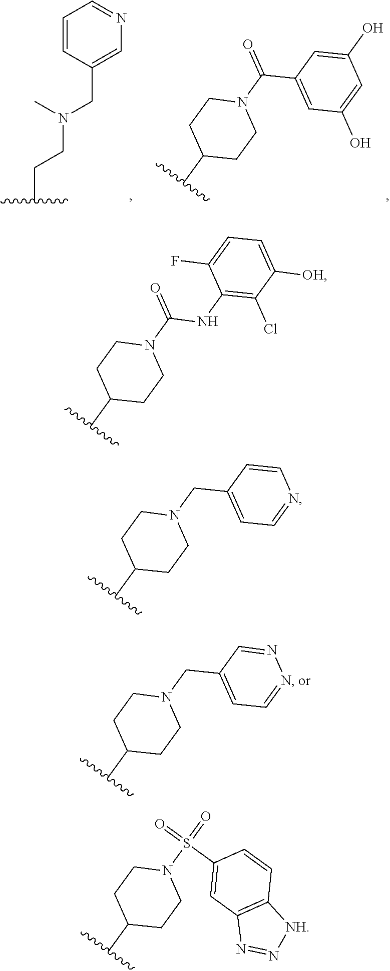

In some embodiments of a compound of Formula (Ia), (Ia-1), (Ib), (Ib-1), (IIa), or (IIb), R¹ is hydrogen or C₁-C₆alkyl. In some embodiments of a compound of Formula (Ia), (Ia-1), (Ib), (Ib-1), (IIa), or (IIb), R¹ is hydrogen.

In some embodiments of a compound of Formula (Ia), (Ia-1), (Ib), (Ib-1), (IIa), or (IIb), R^{a} is hydrogen.

In some embodiments of a compound of Formula (Ia), (Ia-1), (Ib), (Ib-1), (IIa), or (IIb), each R^{e} is hydrogen.

In some embodiments of a compound of Formula (Ia), (Ia-1), (Ib), (Ib-1), (IIa), or (IIb), X¹ and X² are —OH.

In some embodiments of a compound of Formula (Ia), (Ia-1), (Ib), (Ib-1), (IIa), or (IIb), each R is independently deuterium, halogen, —CN, —OH, —OR^{a}, —NR^{c}R^{d}, —C(═O)R^{a}, —C(═O)OR^{a}, C₁-C₆ alkyl, C₁-C₆ haloalkyl, C₁-C₆deuteroalkyl, C₁-C₆ hydroxyalkyl, or C₁-C₆ aminoalkyl. In some embodiments of a compound of Formula (Ia), (Ia-1), (Ib), (Ib-1), (IIa), or (IIb), each R is independently deuterium, halogen, —CN, —OH, —OR^{a}, —NR^{c}R^{d}, C₁-C₆ alkyl, or C₁-C₆ haloalkyl.

In some embodiments of a compound of Formula (Ia), (Ia-1), (Ib), (Ib-1), (IIa), or (IIb), each R is independently deuterium, halogen, C₁-C₆ alkyl, or C₁-C₆ haloalkyl. In some embodiments of a compound of Formula (Ia), (Ia-1), (Ib), (Ib-1), (IIa), or (IIb), each R is independently halogen.

In some embodiments of a compound of Formula (Ia), (Ia-1), (Ib), (Ib-1), (IIa), or (IIb), m is 0 or 1. In some embodiments of a compound of Formula (Ia), (Ia-1), (Ib), (Ib-1), (IIa), or (IIb), m is 1 or 2. In some embodiments of a compound of Formula (Ia), (Ia-1), (Ib), (Ib-1), (IIa), or (IIb), m is 0. In some embodiments of a compound of Formula (Ia), (Ia-1), (Ib), (Ib-1), (IIa), or (IIb), m is 1. In some embodiments of a compound of Formula (Ia), (Ia-1), (Ib), (Ib-1), (IIa), or (IIb), m is 2.

In some embodiments of a compound of Formula (Ia), (Ia-1), (Ib), (Ib-1), (IIa), or (IIb), Z is hydrogen. In some embodiments of a compound of Formula (Ia), (Ia-1), (Ib), (Ib-1), (IIa), or (IIb), Z is R¹¹; and R¹¹ is C₁-C₆ alkyl. In some embodiments of a compound of Formula (Ia), (Ia-1), (Ib), (Ib-1), (IIa), or (IIb), Z is —R¹⁰OC(═O)R¹¹ or —R¹⁰OC(═O)OR¹¹; R¹⁰ is —CH₂— or —CH(CH₃)—; and R¹¹ is alkyl, cycloalkyl, or heterocycloalkyl.

In some embodiments of a compound of Formula (Ia), (Ia-1), (Ib), (Ib-1), (IIa), or (IIb), Y is absent. In some embodiments of a compound of Formula (Ia), (Ia-1), (Ib), (Ib-1), (IIa), or (IIb), Y is C₁-C₆ alkylene optionally substituted with one or more deuterium, halogen, —CN, —OH, or —OR^{a}. In some embodiments of a compound of Formula (Ia), (Ia-1), (Ib), (Ib-1), (IIa), or (IIb), Y is C₁-C₆ alkylene. In some embodiments of a compound of Formula (Ia), (Ia-1), (Ib), (Ib-1), (IIa), or (IIb), Y is C₁-C₄ alkylene. In some embodiments of a compound of Formula (Ia), (Ia-1), (Ib), (Ib-1), (IIa), or (IIb), Y is C₂-C₆ alkylene. In some embodiments of a compound of Formula (Ia), (ha-1), (Ib), (Ib-1), (IIa), or (IIb), Y is C₂-C₄ alkylene. In some embodiments of a compound of Formula (Ia), (Ia-1), (Ib), (Ib-1), (IIa), or (IIb), Y is C₁ alkylene.

In some embodiments of a compound disclosed herein, each R^{a} is independently C₁-C₆alkyl, C₁-C₆haloalkyl, C₁-C₆deuteroalkyl, C₁-C₆hydroxyalkyl, C₁-C₆aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{a} is independently C₁-C₆alkyl, C₁-C₆haloalkyl, —Y-cycloalkyl, or —Y-heterocycloalkyl; wherein each alkyl, cycloalkyl, and heterocycloalkyl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{a} is independently C₁-C₆alkyl, C₁-C₆haloalkyl, —Y-cycloalkyl, or —Y-heterocycloalkyl. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl. In some embodiments of a compound disclosed herein, each R^{a} is independently —Y-aryl; wherein each aryl is independently optionally substituted with one or more halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{a} is independently —Y-aryl; wherein each aryl is independently optionally substituted with one or more halogen, —CN, —OH, —OCH₃, —NH₂, —C₁-C₆alkyl, or C₁-C₆haloalkyl. In some embodiments of a compound disclosed herein, each R^{a} is independently —Y-aryl; wherein each aryl is independently optionally substituted with one or more halogen or —OH.

In some embodiments of a compound disclosed herein, each R^{b} is independently hydrogen, C₁-C₆alkyl, C₁-C₆haloalkyl, C₁-C₆deuteroalkyl, C₁-C₆hydroxyalkyl, C₁-C₆aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{b} is independently hydrogen, C₁-C₆alkyl, C₁-C₆haloalkyl, —Y-cycloalkyl, or —Y-heterocycloalkyl; wherein each alkyl, cycloalkyl, and heterocycloalkyl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{b} is independently hydrogen, C₁-C₆alkyl, C₁-C₆haloalkyl, —Y-cycloalkyl, or —Y-heterocycloalkyl. In some embodiments of a compound disclosed herein, each R^{b} is independently hydrogen, C₁-C₆alkyl or C₁-C₆haloalkyl. In some embodiments of a compound disclosed herein, each R^{b} is independently hydrogen or C₁-C₆alkyl. In some embodiments of a compound disclosed herein, each R^{b} is hydrogen. In some embodiments of a compound disclosed herein, each R^{b} is independently C₁-C₆alkyl.

In some embodiments of a compound disclosed herein, each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R^{c} and R^{d} are independently hydrogen, C₁-C₆alkyl, C₁-C₆haloalkyl, —Y-cycloalkyl, or —Y-heterocycloalkyl; wherein each alkyl, cycloalkyl, and heterocycloalkyl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl. In some embodiments of a compound disclosed herein, each R and R^{a} are independently hydrogen, C₁-C₆alkyl, C₁-C₆haloalkyl, —Y-cycloalkyl, or —Y-heterocycloalkyl. In some embodiments of a compound disclosed herein, each R^{c} and R^{d} are independently hydrogen, C₁-C₆alkyl, or C₁-C₆haloalkyl. In some embodiments of a compound disclosed herein, each R^{c} and R^{d} are independently hydrogen or C₁-C₆alkyl. In some embodiments of a compound disclosed herein, each R^{c} and R^{d} are hydrogen. In some embodiments of a compound disclosed herein, each R^{c} and R^{d} are independently C₁-C₆alkyl.

In some embodiments of a compound disclosed herein, R^{c} and R^{d} are taken together with the atom to which they are attached to form a heterocycloalkyl optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl.

In some embodiments of a compound disclosed herein, when a substituent is optionally substituted with one or more substituents as defined herein, the substituent is optionally substituted with 1-6 substituents as defined herein. In some embodiments of a compound disclosed herein, when a substituent is optionally substituted with one or more substituents as defined herein, the substituent is optionally substituted with 1-5 substituents as defined herein. In some embodiments of a compound disclosed herein, when a substituent is optionally substituted with one or more substituents as defined herein, the substituent is optionally substituted with 1-4 substituents as defined herein. In some embodiments of a compound disclosed herein, when a substituent is optionally substituted with one or more substituents as defined herein, the substituent is optionally substituted with 1-3 substituents as defined herein. In some embodiments of a compound disclosed herein, when a substituent is optionally substituted with one or more substituents as defined herein, the substituent is optionally substituted with 1 or 2 substituents as defined herein. In some embodiments of a compound disclosed herein, when a substituent is optionally substituted with one or more substituents as defined herein, the substituent is optionally substituted with 1 substituent as defined herein. In some embodiments of a compound disclosed herein, when a substituent is optionally substituted with one or more substituents as defined herein, the substituent is optionally substituted with 2 substituents as defined herein. In some embodiments of a compound disclosed herein, when a substituent is optionally substituted with one or more substituents as defined herein, the substituent is optionally substituted with 3 substituents as defined herein. In some embodiments of a compound disclosed herein, when a substituent is optionally substituted with one or more substituents as defined herein, the substituent is optionally substituted with 4 substituents as defined herein. In some embodiments of a compound disclosed herein, when a substituent is optionally substituted with one or more substituents as defined herein, the substituent is optionally substituted with 5 substituents as defined herein.

### Further Forms of Compounds Disclosed Herein

### Isomers/Stereoisomers

In some embodiments, due to the oxophilic nature of the boron atom, the compounds described herein may convert to, or exist in equilibrium with, alternate forms, particularly in milieu that contain water (aqueous solution, plasma, etc.). Accordingly, the compounds described herein may exist in an equilibrium between the “closed” cyclic form shown in Formula (Ia), (Ia′), (IIa) and the “open” acyclic form shown in Formula (Ib), (Ib′), (IIb). In addition the compounds described herein may associate into intramolecular dimers, trimers, and related combinations.

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization. Compounds described herein may be prepared as a single isomer or a mixture of isomers.

### Tautomers

In some situations, compounds described herein exist as tautomers. The compounds described herein include all possible tautomers within the formulas described herein. A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorous, sulfur, fluorine, and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate, or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylateundeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid. In some embodiments, other acids, such as oxalic, while not in themselves pharmaceutically acceptable, are employed in the preparation of salts useful as intermediates in obtaining the compounds described herein and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared from an aqueous/organic solvent mixture, using organic solvents including, but not limited to, dioxane, tetrahydrofuran, or methanol. In addition, the compounds provided herein can exist in unsolvated as well as solvated forms. In general, the solvated forms are considered equivalent to the unsolvated forms for the purposes of the compounds and methods provided herein.

### Pharmaceutical Compositions/Formulations

In another aspect, provided herein are pharmaceutical compositions comprising a compound described herein, or a pharmaceutically acceptable salt, solvate, stereoisomer, tautomer, N-oxide, dimer, or trimer thereof, and a pharmaceutically acceptable excipient. In some embodiments, the pharmaceutical composition further comprises a beta-lactam antibiotic. In certain embodiments, the beta-lactam antibiotic is a penicillin, cephalosporin, carbapenem, monobactam, bridged monobactam, or a combination thereof.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound described herein with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

### Combination Treatment

The compounds described herein may be used in combination with one or more antibiotics in the treatment of bacterial infections. Such antibiotics may be administered, by a route and in an amount commonly used therefore, contemporaneously or sequentially with a compound described herein. When a compound described herein is used contemporaneously with one or more antibiotic, a pharmaceutical composition in unit dosage form containing such other drugs and the compound of the present invention is preferred. However, the combination therapy may also include therapies in which the compound described herein and one or more antibiotic are administered on different overlapping schedules. It is also contemplated that when used in combination with one or more antibiotics, the antibiotics may be used in lower doses than when each is used singly.

Accordingly, the pharmaceutical compositions of the present invention also include those that contain one or more antibiotics, in addition to a compound described herein. In some embodiments, a pharmaceutical composition comprising a compound described herein further comprises a beta-lactam antibiotic. In certain embodiments, the beta-lactam antibiotic is a penicillin, cephalosporin, carbapenem, monobactam, bridged monobactam, or a combination thereof.

In some embodiments, the compounds described herein are used in combination with one or more antibiotics in the treatment of bacterial infections. In certain embodiments, the bacterial infection is a upper or lower respiratory tract infection, a urinary tract infection, an intra-abdominal infection, or a skin infection. In some embodiments, the bacterial infection is an upper or lower respiratory tract infection, a urinary tract infection, an intra-abdominal infection, or a skin infection. In some embodiments, the bacterial infection is uncomplicated or complicated urinary tract infections, uncomplicated or complicated gonorrhea, upper or lower respiratory tract infections, skin or skin structure infections, intra-abdominal infections, central nervous system infections, blood stream infections, or systemic infections.

In some embodiments, the one or more antibiotics are selected from β-lactam antibiotics. β-Lactam antibiotics include, but are not limited to, penicillins, penems, carbapenems, cephalosporins, cephamycins, monobactams, or combinations thereof. Penicillins include, but are not limited to, amoxicillin, ampicillin, azidocillin, azlocillin, bacampicillin, benzathinebenzylpenicillin, benzathinephenoxymethylpenicillin, benzylpenicillin (G), carbenicillin, carindacillin, clometocillin, cloxacillin, dicloxacillin, epicillin, flucloxacillin, hetacillin, mecillinam, metampicillin, meticillin, mezlocillin, nafcillin, oxacillin, penamecillin, pheneticillin, phenoxymethylpenicillin (V), piperacillin, pivampicillin, pivmecillinam, procaine benzylpenicillin, propicillin, sulbenicillin, talampicillin, temocillin, and ticarcillin. Penems include, but are not limited to, faropenem. Carbapenems include, but are not limited to, biapenem, ertapenem, doripenem, imipenem, meropenem, and panipenem. Cephalosporins/Cephamycins include, but are not limited to, cefacetrile, cefaclor, cefadroxil, cefalexin, cefaloglycin, cefalonium, cefaloridine, cefalotin, cefamandole, cefapirin, cefatrizine, cefazaflur, cefazedone, cefazolin, cefbuperazone, cefcapene, cefdaloxime, cefdinir, cefditoren, cefepime, cefetamet, cefixime, cefmenoxime, cefmetazole, cefminox, cefodizime, cefonicid, cefoperazone, ceforanide, cefotaxime, cefotetan, cefotiam, cefovecin, cefoxitin, cefozopran, cefpimizole, cefpiramide, cefpirome, cefpodoxime, cefprozil, cefquinome, cefquinome, cefradine, cefroxadine, cefsulodin, ceftarolinefosamil, ceftazidime, cefteram, ceftezole, ceftibuten, ceftiofur, ceftiolene, ceftizoxime, ceftobiprole, ceftriaxone, cefuroxime, cefuzonam, flomoxef, latamoxef, and loracarbef. Monobactams include, but are not limited to, aztreonam, carumonam, nocardicinA, and tigemonam.

### Methods

The present disclosure also provides methods for inhibiting bacterial growth, such methods comprising contacting a bacterial cell culture, or a bacterially infected cell culture, tissue, or organism, with a penicillin-binding protein inhibitor described herein. Preferably, the bacteria to be inhibited by administration of a penicillin-binding protein inhibitor described herein are bacteria that are resistant to beta-lactam antibiotics. The term “resistant” is well-understood by those of ordinary skill in the art (see, e g Payne et al., Antimicrobial Agents and Chemotherapy 38 767-772 (1994), Hanaki et al., Antimicrobial Agents and Chemotherapy 30 1120-1126 (1995)). In some embodiments, the penicillin-binding protein inhibitor described herein is used to treat a bacterial infection that is resistant to beta-lactam antibiotic. In some embodiments, the penicillin-binding protein inhibitor described herein is used to treat a bacterial infection that has developed beta-lactamase enzymes.

These methods are useful for inhibiting bacterial growth in a variety of contexts. In certain embodiments, a compound described herein is administered to an experimental cell culture in vitro to prevent the growth of beta-lactam resistant bacteria. In some embodiments, a compound described herein is administered to a mammal, including a human, to prevent the growth of beta-lactam resistant bacteria in vivo. The method according to this embodiment comprises administering a therapeutically effective amount of a penicillin-binding protein inhibitor described herein for a therapeutically effective period of time to a mammal, including a human. Preferably, the penicillin-binding protein inhibitor described herein is administered in the form of a pharmaceutical composition as described above.

In another aspect provided herein are methods of treating a bacterial infection, which method comprises administering to a subject a pharmaceutical composition comprising a compound described herein, or a pharmaceutically acceptable salt, solvate, stereoisomer, tautomer, N-oxide, dimer, or trimer thereof, and a pharmaceutically acceptable excipient. In some embodiments, the methods of treating a bacterial infection in a subject comprises administering to the subject a pharmaceutical composition as described herein. In some embodiments, the bacterial infection is an upper or lower respiratory tract infection, a urinary tract infection, an intra-abdominal infection, or a skin infection. In some embodiments, the bacterial infection is an upper or lower respiratory tract infection, a urinary tract infection, an intra-abdominal infection, or a skin infection. In some embodiments, the bacterial infection is uncomplicated or complicated urinary tract infections, uncomplicated or complicated gonorrhea, upper or lower respiratory tract infections, skin or skin structure infections, intra-abdominal infections, central nervous system infections, blood stream infections, or systemic infections.

In some embodiments, the infection that is treated or prevented is cause by a bacteria that includes Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella kingae, Moraxella catarrhalis, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In some embodiments, the infection that is treated or prevented is caused by a bacteria that includes Pseudomonas aeruginosa, Pseudomonas fluorescens, Stenotrophomonas maltophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Acinetobacter calcoaceticus, Acinetobacter haemolyticus, Yersinia enterocolitica, Yersinia pestis, Yersinia pseudotuberculosis, Yersinia intermedia, Haemophilus influenzae, Haemophilus parainfluenzae, Haemophilus haemolyticus, Haemophilus parahaemolyticus, Helicobacter pylori, Campylobacter fetus, Campylobacter jejuni, Campylobacter coli, Vibrio cholerae, Vibrio parahaemolyticus, Legionella pneumophila, Listeria monocytogenes, Neisseria gonorrhoeae, Neisseria meningitidis, Moraxella catarrhalis, Bacteroides fragilis, Bacteroides vulgatus, Bacteroides ovalus, Bacteroides thetaiotaomicron, Bacteroides uniformis, Bacteroides eggerthii, or Bacteroides splanchnicus.

In some embodiments, the infection that is treated or prevented is caused by a Enterobacterales bacteria. In some embodiments, the infection that is treated or prevented is caused by a bacteria that includes Escherichia spp, Klebsiella spp., Enterobacter spp., Citrobacter spp., Morganella spp., Proteus spp., Salmonella spp., Serratia spp., Shigella spp., or Yersinia spp.

In some embodiments, the compounds disclosed herein are useful in the treatment or prevention of infection associated with non-fermenting bacteria. In some embodiments, the compounds disclosed herein are useful in the treatment or prevention of infection associated with non-fermenting gram-negative bacteria. In some embodiments, the non-fermenting gram-negative bacteria is Pseudomonas aeruginosa, Acinetobacter spp. (A. baumannii/A. calcoaceticus), Stenotrophomonas maltophilia, Elizabethkingia spp (E. meningoseptica/E. anophelis, Burkholderia cepacia complex, Burkholderia pseudomallei, or Burkholderia mallei.

In some embodiments, the infection that is treated or prevented is tuberculosis. In some embodiments, the infection that is treated or prevented is caused by Mycobacterium tuberculosis. In some embodiments, the infection that is treated or prevented is caused by a bacteria that is a non-TB mycobacterial species. In some embodiments, the non-TB mycobacterial species is M. abscessus, M. canum, M. bovis, M. africanum, or M. caprae.

In some embodiments, the infection that is treated or prevented is gonorrhea. In some embodiments, the infection that is treated or prevented is caused by Neisseria gonorrhoeae.

In some embodiments, the infection that is treated or prevented is meningitis and other forms of meningococcal disease such as meningococcemia. In some embodiments, the infection that is treated or prevented is caused by Neisseria meningitidis.

In some embodiments, the infection that is treated or prevented is caused by a bacteria that is Neisseria gonorrhoeae. In some embodiments, the infection that is treated or prevented is caused by a bacteria that is Pseudomonas aeruginosa. In some embodiments, the infection that is treated or prevented is caused by a bacteria that is Acinetobacter baumannii. In some embodiments, the infection that is treated or prevented is caused by a bacteria that is Pseudomonas aeruginosa/Acinetobacter baumannii. In some embodiments, the infection that is treated or prevented is caused by a bacteria that is a carbapenem-resistant Enterobacterales (CRE).

In some embodiments of the methods described herein, the compound described herein is not administered with a β-lactam antibiotic. In some embodiments of the methods described herein, the compound described herein is not administered with a β-lactamase inhibitor. In some embodiments of the methods described herein, the compound described herein is not administered with a combination of a β-lactam antibiotic and a β-lactamase inhibitor.

### EXAMPLES

### General Examples for the Preparation of Compounds.

The starting materials and intermediates for the compounds of this invention may be prepared by the application or adaptation of the methods described below, their obvious chemical equivalents, or, for example, as described in literature such as The Science of Synthesis, Volumes 1-8. Editors E. M. Carreira, et al., Thieme publishers (2001-2008). The use of protective groups may be as described in methodology compendia such as Greene's Protective Groups in Organic Synthesis, Fifth Edition. John Wiley & Sons, Inc. 2014.

Certain compounds of Formula I (Scheme 1) are prepared from the corresponding functional-group-protected boronic acid esters A by treatment with a Lewis acid in a solvent such as dichloromethane, at a temperature between −78° C. and 0° C. followed by an aqueous quench.

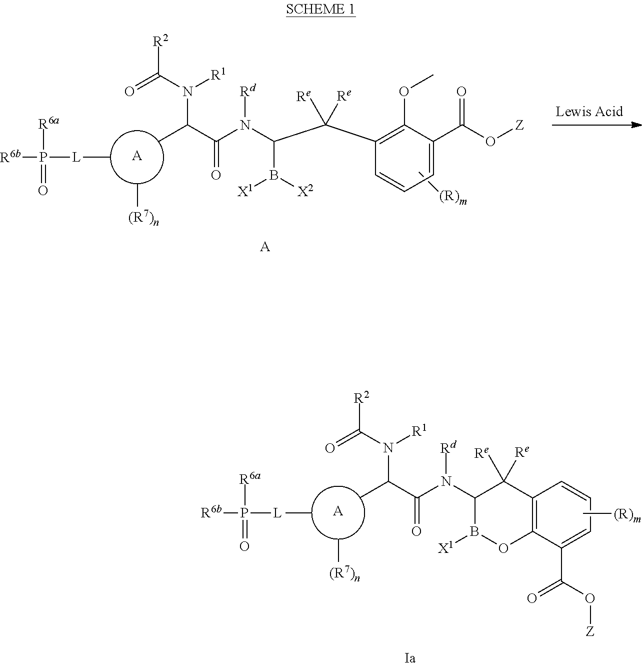

Amide intermediates A may be prepared according to the route outlined in Scheme 2. Chloro-boronates B, prepared by methods described previously (e.g. see WO2014089365), is reacted with silylamine bases such as lithium hexamethyldisilazide, and the intermediate silylamine is treated with carboxylic acids C under amide coupling conditions (such as with carbodiimide dehydrating reagents, HATU, or other coupling reagents) to provide protected amides A. Alternatively, the above silylamine intermediate is allowed to react with acid chlorides to provide A. Carboxylic acids (C) or acid chlorides (D) may be obtained from commercial sources, prepared according to known methods in the literature, or prepared by a number of different reaction sequences. Formation of the acid chloride (D) involves treatment of (C) with a chlorinating agent such as thionyl chloride, phosphorous pentachloride or oxalyl chloride, in a solvent such as dichloromethane, in the presence of a catalyst such as DMF, at around room temperature. In certain cases, DMF is also used as a co-solvent. Formation of the anhydride (E) involves treatment of (C) with a sterically hindered acid chloride or chloroformate, such as trimethylacetyl chloride or isopropylchloroformate, in an inert solvent such as dichloromethane, in the presence of a non-nucleophilic base, such as triethyl amine or diisopropylethylamine at room temperature or below. Formation of the activated ester (F) involves treatment of (C) with an activating reagent system such as EDCI, EDCI/HOBt, DCC/HOBt, HATU, BOP reagents or TBTU, in a solvent such as DMF, DMA, NMP or dichloromethane at room temperature or below (International Journal of Pharmaceutical Sciences Review and Research (2011), 8(1), 108-119).

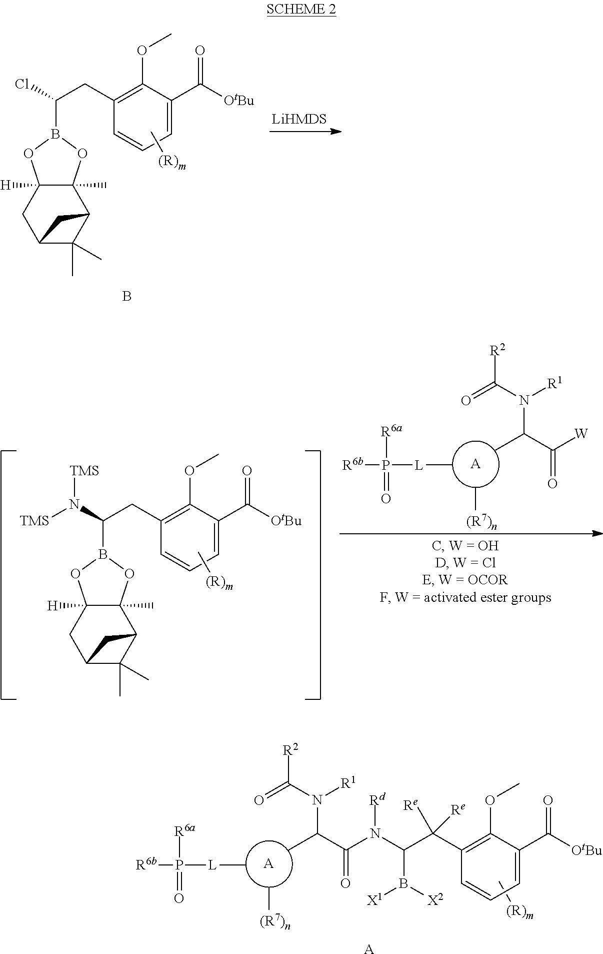

Chloroboronates B may be prepared from aryl halides or aryl triflates K (X═Br, I or OTf) in the manner described in Scheme 4. Compounds K (X═Br, I or OTf) may be converted into boronic acids L by treatment with alkyl lithium reagents, for example n-butyllithium, and then quenching the intermediate aryllithium species with trialkylboronates, followed by aqueous work-up. The boronic acids L may be converted into protected boronate esters M by treatment with 1,2-diols, such as (+)-pinanediol or pinacol. Alternatively, aryl halides K may be converted to boronate esters M by transition-metal-catalyzed reaction with diboron compounds, for example bis[(+)-pinanediolato]diboron and palladium catalysts. Two sequential Matteson reactions, as described previously, provide chloroboronates B bearing a wide range of substituents R^{a}, R^{b}, and R^{c}. Another variant consists of reaction of K with chloromethyl boronate J and isopropylmagnesium chloride to provide desired intermediate N directly.

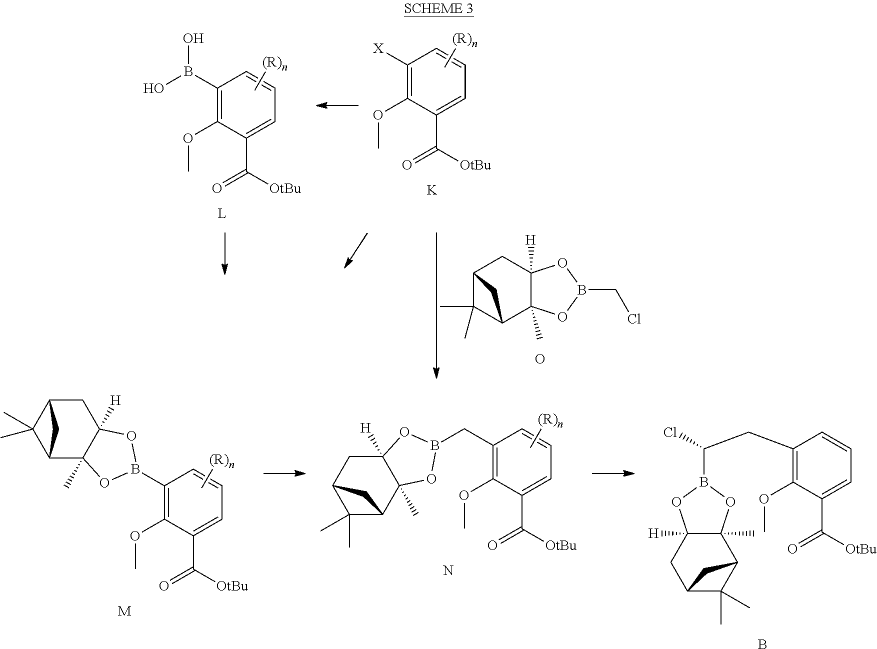

While there are common themes and strategies among the illustrative examples cited below, the selection of an appropriate reaction sequence (including protecting group requirements) is dictated by the nature and arrangement of the functionality present in the target molecule and, therefore, may involve obvious adaptations of the illustrated methods in order to be applied in a particular case.

General Method A: Deprotection with Boron Trichloride or Boron Tribromide.

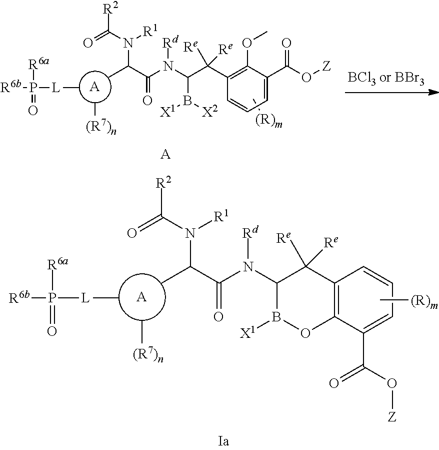

To a solution of the protected precursor A (0.4 mmol) in anhydrous DCM (15 mL) at −78° C. under argon was added dropwise BCl₃ or BBr₃ (1.0 M in DCM, 2.4-4 mL, 2.4-4 mmol, 6-10 equivalents). The reaction mixture was allowed to slowly warmed to 0° C. over 1 h, and stirred between 0-5° C. for an additional 1-2 h, then quenched with water (2 mL) and methanol (20 mL), evaporated to remove DCM, washed with hexane, and concentrated to a volume of ˜4-5 mL. The crude product was purified by reverse phase preparative HPLC and dried using lyophilization to afford the product I.

General Method B: Deprotection with Aluminum Chloride.

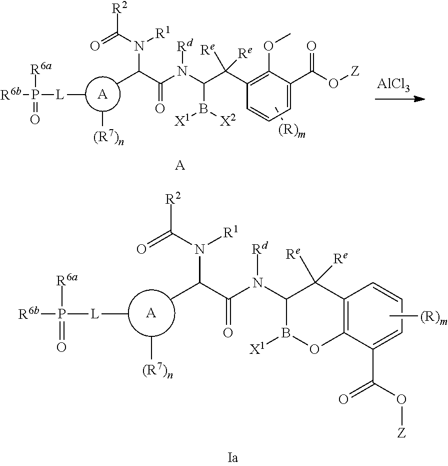

To a solution of the protected precursor A (0.4 mmol) in anhydrous DCM (15 mL) was added AlCl₃ (535 mg, 4 mmol, 10 equivalents) in one portion at RT. The reaction mixture was stirred at RT for 24 h, then quenched with water (2 mL) and methanol (20 mL), evaporated to remove DCM, and washed with hexane, and concentrated to a volume of ˜4-5 mL. The crude product was purified by reverse phase preparative HPLC and dried using lyophilization to afford the product I.

### General Method C: Conversion of Chloro-Boronates to Amides.

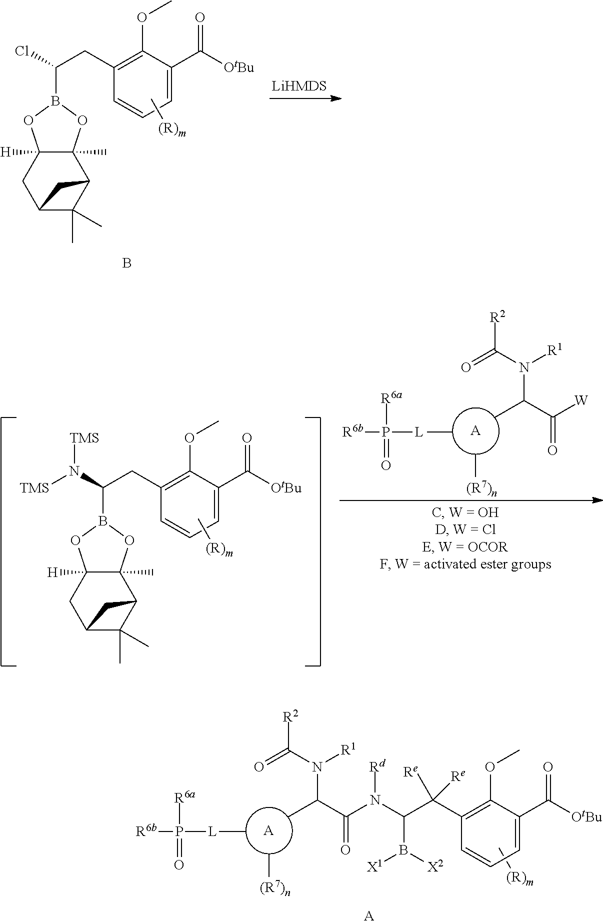

To a solution of the chloride B (4 mmol) in anhydrous THF (16 mL) was added dropwise LiHMDS (1.0 M in THF, 4.5 mL, 4.5 mmol) at −60° C. under argon. The reaction mixture was allowed to slowly warmed to 0° C. over 45 min, and stirred at RT for an additional 2 h.

In a separate flask was charged the carboxylic acid C (4.2 mmol) and anhydrous DMA (20 mL), to this mixture was added HATU (1.68 g, 4.4 mmol) followed by 4-methylmorpholine (0.49 mL, 4.4 mmol). The reaction mixture was stirred at RT for 2 h, at which time the solution from the above reaction was added to the flask, and the reaction mixture was stirred at RT overnight, then diluted with EtOAc, washed with water, brine, and dried over Na₂SO₄, concentrated in vacuo to afford the crude product, which was purified by flash chromatography on silica gel (hexane-EtOAc, 20:1-1:1, or hexane-acetone, 10:1-1:1, or DCM-MeOH, 30:1-10:1) to afford the product A.

### Example 1: Synthesis of (3R)-3-(2-(4-ethyl-2,3-dioxopiperazine-1-carboxamido)-2-(2-fluoro-4-phosphonophenyl)acetamido)-2-hydroxy-3,4-dihydro-2H-benzo[e][1,2]oxaborinine-8-carboxylic acid

### Step 1: Synthesis of 2-((tert-butoxycarbonyl)amino)-2-(2-fluoro-4-iodophenyl)acetic acid

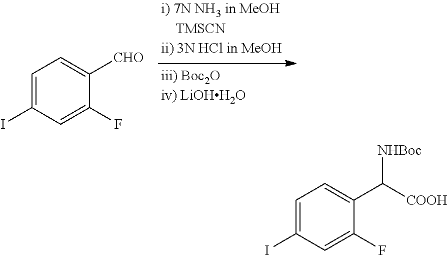 [SMILES: O=Cc1ccc(I)cc1F ; CC(C)(C)OC(=O)NC(C(=O)O)c1ccc(I)cc1F]

To 2-fluoro-4-iodobenzaldehyde 1 g (4 mmol) at 0° C. was added 7 N ammonia in methanol (23 mL), followed by trimethylsilyl cyanide 0.75 mL (6 mmol, 1.5 eq), stirred at 45° C. for 7 h, and concentrated in vacuo. The crude product was dissolved in 3 N hydrochloric acid in methanol (14 mL), stirred at 70° C. for 18 h, and concentrated in vacuo to give the HCl salt. The reaction was slurried in tetrahydrofuran (20 mL) and cooled at 0° C. Triethylamine 1.7 mL (12 mmol, 3 eq) was added, followed by di-tert-butyl dicarbonate 1.3 g (6 mmol, 1.5 eq), warmed at RT for 1 h, and concentrated in vacuo. The product was purified by flash chromatography on silica gel (10% ethyl acetate/hexanes) to give the desired product, 1.1 g. To methyl 2-((tert-butoxycarbonyl)amino)-2-(2-fluoro-4-iodophenyl)acetate 1.1 g (2.71 mmol) was dissolved in tetrahydrofuran (10 mL)/H₂O (10 mL), followed by lithium hydroxide monohydrate 0.34 g (8.14 mmol, 3 eq) and was stirred at RT for 1 h, and concentrated. 2 N Hydrochloric acid in water was added drop wise to the solution to obtain pH 2, diluted with dichloromethane, and extracted. The organic layer was washed with water, dried over sodium sulfate, and concentrated to give the title compound, 1.1 g, (68% overall yield). ESI-MS m/z 396 (M+H)⁺.

### Step 2: Synthesis of tert-butyl 3-((2R)-2-(2-((tert-butoxycarbonyl)amino)-2-(2-fluoro-4-iodophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

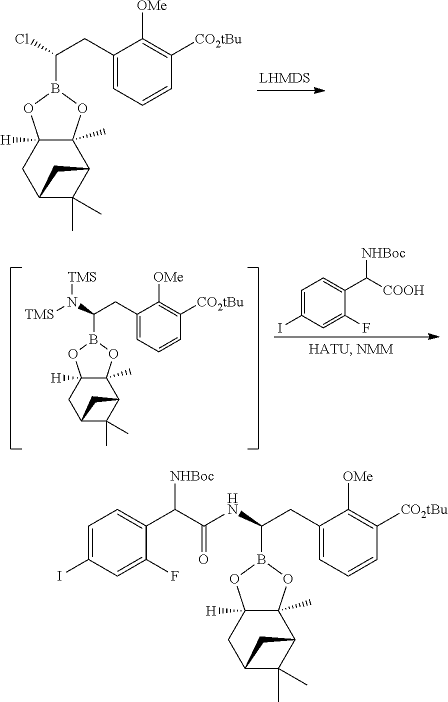

By following the General procedure C, the chloride (prepared as previous reported, WO 2014/089365) was treated with LiHMDS, and then coupled with 2-((tert-butoxycarbonyl)amino)-2-(2-fluoro-4-iodophenyl)acetic acid in the presence of HATU and NMM, yielding the title compound. ESI-MS m/z 807 (M+H)⁺.

### Step 3: Synthesis of tert-butyl 3-((2R)-2-(2-((tert-butoxycarbonyl)amino)-2-(2-fluoro-4-iodophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

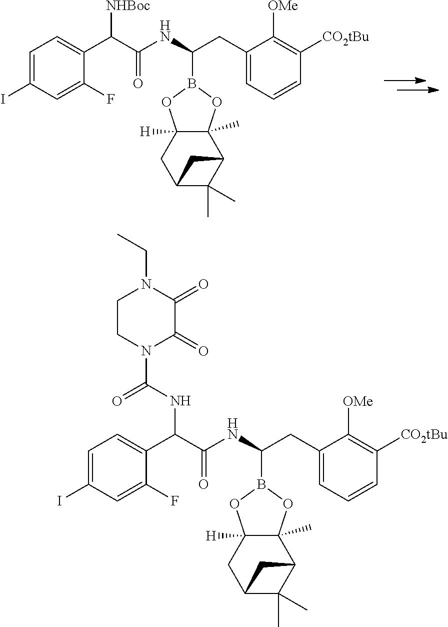 [SMILES: COc1c(C[C@H](NC(=O)C(NC(=O)OC(C)(C)C)c2ccc(I)cc2F)B2O[C@@H]3C[C@@H]4C[C@@H](C4(C)C)[C@]3(C)O2)cccc1C(=O)OC(C)(C)C ; CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(I)cc2F)C(=O)C1=O]

To tert-butyl 3-((2R)-2-(2-((tert-butoxycarbonyl)amino)-2-(2-fluoro-4-iodophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate 0.64 g (0.79 mmol) at 0° C. was added 1 N hydrochloric acid in diethyl ether (28 mL) and warmed at RT for 18 h. The reaction was heated at 40° C. for 5 h and concentrated in vacuo to give tert-butyl 3-((2R)-2-(2-amino-2-(2-fluoro-4-iodophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate hydrogen chloride. ESI-MS m/z 707 (M+H)⁺.

To tert-butyl 3-((2R)-2-(2-amino-2-(2-fluoro-4-iodophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate hydrogen chloride 0.59 g (0.8 mmol) in dichloromethane (13 mL) at 0° C. was added N,N-diisopropylethylamine 0.41 mL (2.38 mmol, 3 eq), followed by 4-ethyl-2,3-dioxopiperazine-1-carbonyl chloride 0.24 g (1.2 mmol, 1.5 eq) and the reaction was warmed at RT for 30 min. The product was quenched with water, washed with brine, dried over sodium sulfate, and concentrated to give the title compound taken directly on to the next step without further purification. 0.76 g. ESI-MS m/z 875 (M+H)⁺.

### Step 4: Synthesis of tert-butyl 3-((2R)-2-(2-(4-(bis(benzyloxy)phosphoryl)-2-fluorophenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate

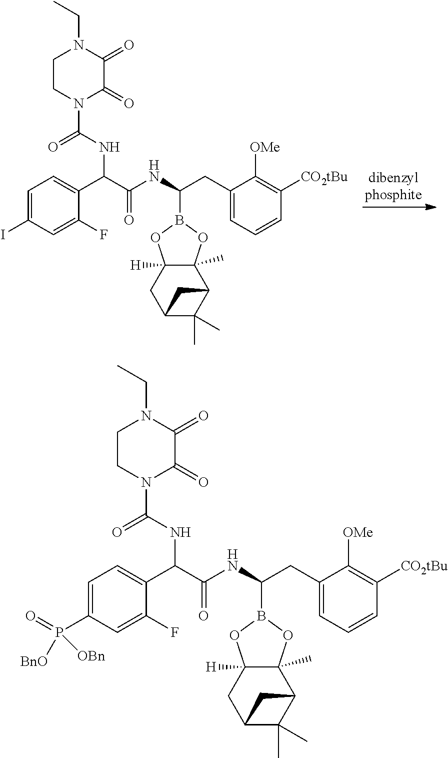 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(I)cc2F)C(=O)C1=O ; CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(P(=O)(OCc3ccccc3)OCc3ccccc3)cc2F)C(=O)C1=O]

To tert-butyl 3-((2R)-2-(2-(4-ethyl-2,3-dioxopiperazine-1-carboxamido)-2-(2-fluoro-4-iodophenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate 0.76 g (0.86 mmol) was added diisopropylethylamine 0.45 mL (2.59 mmol, 3 eq), tetrakis(triphenylphosphine)palladium(0) 0.1 g, (0.09 mmol, 10 mol %), dibenzyl phosphite 0.38 mL (1.73 mmol, 2 eq), followed by 1-methyl-2-pyrrolidinone (15 mL) and degassed 3× under argon. The mixture was stirred at 90° C. for 1 h, cooled, diluted with ethyl acetate, washed with water 3×, dried over sodium sulfate, and concentrated to give the title compound taken directly on to the next step without further purification. ESI-MS m/z 1009 (M+H)⁺.

### Step 5: Synthesis of (3R)-3-(2-(4-ethyl-2,3-dioxopiperazine-1-carboxamido)-2-(2-fluoro-4-phosphonophenyl)acetamido)-2-hydroxy-3,4-dihydro-2H-benzo[e][1,2]oxaborinine-8-carboxylic acid

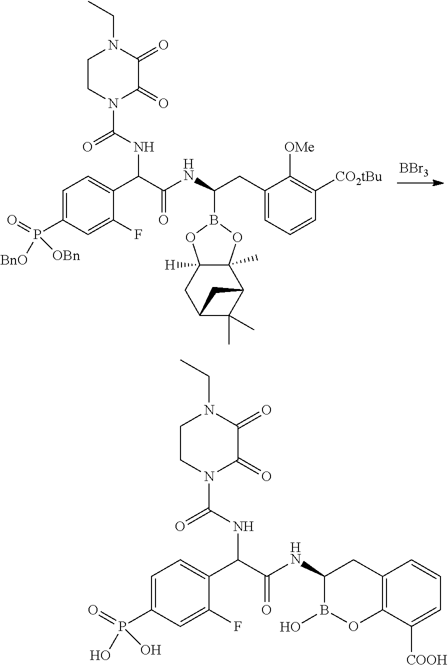

To tert-butyl 3-((2R)-2-(2-(4-(bis(benzyloxy)phosphoryl)-2-fluorophenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate 0.87 g (0.86 mmol) in dichloromethane (25 mL) at −78° C. was added 1M boron tribromide in dichloromethane 8.6 mL (8.6 mmol, 10 eq) drop wise and warmed at RT for 30 min. The crude mixture was cooled at 0° C., quenched with H₂O/MeOH, and concentrated. The title compound was purified by reversed phase HPLC. ESI-MS m/z 607 (M+H)⁺.

### Example 2: Synthesis of (3R)-3-(2-(4-ethyl-2,3-dioxopiperazine-1-carboxamido)-2-(2-fluoro-5-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing 2-fluoro-5-iodobenzaldehyde in place of 2-fluoro-4-iodobenzaldehyde in Step 1. ESI-MS m/z 607 (M+H)⁺.

### Example 3: Synthesis of (3R)-3-(2-(4-(2-aminoethyl)-2,3-dioxopiperazine-1-carboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing tert-butyl (2-(4-(chlorocarbonyl)-2,3-dioxopiperazin-1-yl)ethyl)carbamate in place of 4-ethyl-2,3-dioxopiperazine-1-carbonyl chloride in Step 3. ESI-MS m/z 604 (M+H)⁺.

### Example 4: Synthesis of (3R)-3-(2-(2,3-dioxo-4-(2,2,2-trifluoroethyl)piperazine-1-carboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing 2,3-dioxo-4-(2,2,2-trifluoroethyl)piperazine-1-carbonyl chloride in place of 4-ethyl-2,3-dioxopiperazine-1-carbonyl chloride in Step 3. ESI-MS m/z 643 (M+H)⁺.

### Example 5: Synthesis of (R)-2-hydroxy-3-((S)-2-(3-(methylsulfonyl)-2-oxoimidazolidine-1-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 1, utilizing 3-(methylsulfonyl)-2-oxoimidazolidine-1-carbonyl chloride in place of 4-ethyl-2,3-dioxopiperazine-1-carbonyl chloride in Step 3. After reversed phase HPLC purification in Step 5, the title compound was collected as the first eluting peak. ESI-MS m/z 611 (M+H)⁺.

### Example 6: Synthesis of (R)-2-hydroxy-3-((R)-2-(3-(methylsulfonyl)-2-oxoimidazolidine-1-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 1, utilizing 3-(methylsulfonyl)-2-oxoimidazolidine-1-carbonyl chloride in place of 4-ethyl-2,3-dioxopiperazine-1-carbonyl chloride in Step 3. After reversed phase HPLC purification in Step 5, the title compound was collected as the second eluting peak. ESI-MS m/z 611 (M+H)⁺.

### Example 7: Synthesis of (R)-3-((R)-2-(4-ethyl-2,3-dioxopiperazine-1-carboxamido)-2-(3-fluoro-4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing 3-fluoro-4-iodobenzaldehyde in place of 2-fluoro-4-iodobenzaldehyde in Step 1. After reversed phase HPLC purification in Step 5, the title compound was collected as the second eluting peak. ESI-MS m/z 607 (M+H)⁺.

### Example 8: Synthesis of (3R)-3-(2-(4-(2-chloro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 2-(4-(bis(benzyloxy)phosphoryl)phenyl)-2-((tert-butoxycarbonyl)amino)acetic acid

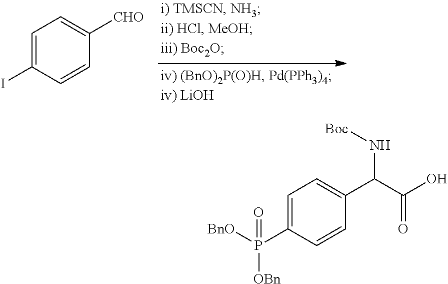 [SMILES: O=Cc1ccc(I)cc1 ; CC(C)(C)OC(=O)NC(C(=O)O)c1ccc(P(=O)(OCc2ccccc2)OCc2ccccc2)cc1]

### Step 1a.

To 4-iodobenzaldehyde (46.2 g, 200 mmol) at 0° C. was added 7 N ammonia in methanol (600 mL), followed by trimethylsilyl cyanide (29.8 g, 300 mmol, 1.5 eq). The mixture was stirred at 45° C. for 24 h and concentrated in vacuo.

### Step 1b.

The crude product was dissolved in 3 N hydrochloric acid in methanol (400 mL), stirred at 50° C. for 36 h and concentrated in vacuo.

### Step 1c.

To the crude product in DCM (400 mL) at 0° C. was added triethylamine (30.3 g, 300 mmol, 3 eq) followed by di-tert-butyl dicarbonate (65.4 g, 300 mmol, 1.5 eq). The reaction was warmed to RT for 12 h and concentrated in vacuo. The crude product was purified by flash silica gel chromatography (10% ethyl acetate/hexanes) to give the desired intermediate (51 g, 66% for 3 steps). Step id.

To methyl 2-((tert-butoxycarbonyl)amino)-2-(4-iodophenyl)acetate (20 g, 52 mmol) was added N,N-diisopropylethylamine 20.2 g, 156 mmol, 3 eq), Pd(PPh₃)₄ (11.8 g, 10.2 mmol, 20 mol %), dibenzyl phosphite (26.7 g, 102 mmol, 2 eq), followed by anhydrous toluene (400 mL) and the mixture was stirred at RT for 5-7 h under argon. The reaction was diluted with ethyl acetate, washed with water then brine, dried over sodium sulfate, and concentrated. The product was purified by flash chromatography on silica gel (35-50% ethyl acetate/hexanes) to give the desired intermediate (27 g, 99%).

### Step 1e.

To methyl 2-(4-(bis(benzyloxy)phosphoryl)phenyl)-2-((tert-butoxycarbonyl)amino)acetate (27 g, 51.4 mmol) in THF (100 mL)/H₂O (100 mL) was added lithium hydroxide monohydrate (6.48 g, 154 mmol, 3 eq) and the mixture was stirred at RT for 1 h. 2N Hydrochloric acid was added drop wise to obtain pH 2 and extracted with dichloromethane. The organic layer was washed with water, dried over sodium sulfate, and concentrated to give 20 g crude product. The product was purified by flash chromatography on silica gel (10-20% MeOH/DCM) to give the title compound (15.8 g, 60%) as a white solid. ESI-MS m/z 512 (M+H)⁺.

### Step 2. Synthesis of 4-(2-chloro-3,4-dimethoxybenzyl)-2,3-dioxopiperazine-1-carbonyl chloride

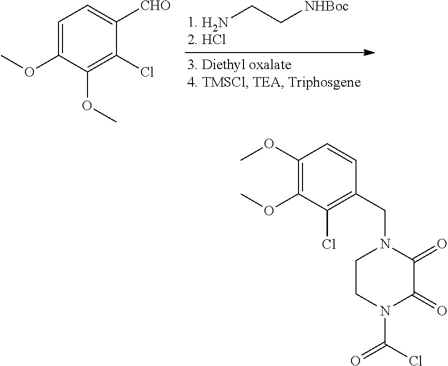 [SMILES: COc1ccc(C=O)c(Cl)c1OC ; CC(C)(C)OC(=O)NCCN ; COc1ccc(CN2CCN(C(=O)Cl)C(=O)C2=O)c(Cl)c1OC]

### Step 2a.

To a solution of 2-chloro-3,4-dimethoxybenzaldehyde (3.0 g, 15 mmol) in MeOH (500 mL) was added tert-butyl (2-aminoethyl)carbamate (2.6 mL, 16 mmol, 1.1 equiv.). The solution was stirred at RT for 2 h then cooled to 0° C. NaBH₄ (2.8 g, 75 mmol, 5.0 equiv.) was added portion wise and allowed to warm to RT overnight. The mixture was concentrated, followed by H₂O (200 mL) and DCM (200 mL) addition. The layers were separated and the aq. layer was extracted with DCM (3×100 mL). The combined organic layers were dried (Na₂SO₄), filtered, and concentrated.

### Step 2b.

To the crude product in MeOH (30 mL) at 0° C. was added HCl (4 M in dioxane, 15 mL). The reaction was warmed to RT and stirred for 2 h before being concentrated in vacuo.

### Step 2c.

The crude product was dissolved in EtOH (150 mL) and TEA (6.3 mL, 45 mmol, 3.0 equiv.), and diethyl oxalate (2.2 mL, 16 mmol, 1.1 equiv.) were added sequentially. The mixture was heated to 90c overnight and concentrated. The crude product was purified by silica gel chromatography (0-5% MeOH/DCM) to yield desired intermediate (2.0 g, 45% over 3 steps).

### Step 2d.

A solution of 1-(2-chloro-3,4-dimethoxybenzyl)piperazine-2,3-dione (2.0 g, 6.7 mmol) in DCM (10 mL)/THF (10 mL) was cooled to −40° C. TMSCl (0.93 mL, 7.4 mmol, 1.1 equiv.) and TEA (1.1 mL, 8.0 mmol, 1.2 equiv.) were added sequentially. The mixture was warmed to 0° C. for 1 h. Triphosgene (795 mg, 2.7 mmol, 0.4 equiv.) in THF (10 mL) was added and warmed to RT for 1 h. The heterogeneous mixture was filtered through Celite and concentrated in vacuo to provide a sticky brown oil. Et₂O (50 mL) was added and the product was triturated at RT overnight and filtered. The resulting solid was washed with Et₂O (10 mL) and dried in vacuo to provide the title compound (2.0 g, 84%).

### Step 3. Synthesis of 2-(4-(bis(benzyloxy)phosphoryl)phenyl)-2-(4-(2-chloro-3,4-dimethoxybenzyl)-2,3-dioxopiperazine-1-carboxamido)acetic acid

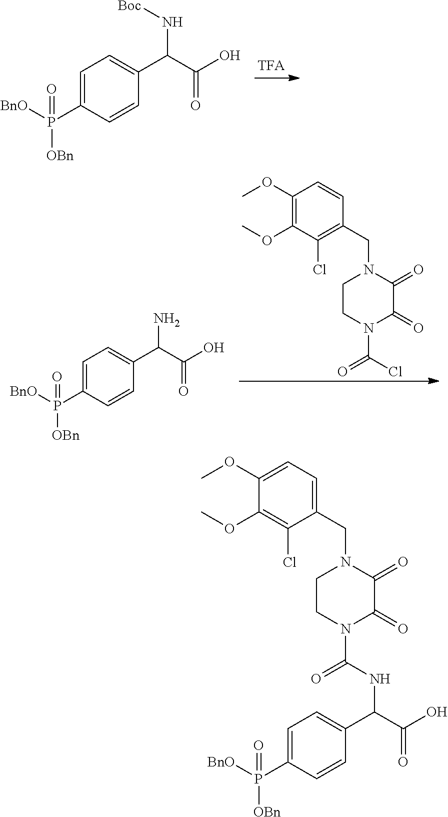 [SMILES: CC(C)(C)OC(=O)NC(C(=O)O)c1ccc(P(=O)(OCc2ccccc2)OCc2ccccc2)cc1 ; NC(C(=O)O)c1ccc(P(=O)(OCc2ccccc2)OCc2ccccc2)cc1 ; COc1ccc(CN2CCN(C(=O)Cl)C(=O)C2=O)c(Cl)c1OC ; COc1ccc(CN2CCN(C(=O)NC(C(=O)O)c3ccc(P(=O)(OCc4ccccc4)OCc4ccccc4)cc3)C(=O)C2=O)c(Cl)c1OC]

### Step 3a.

To a solution of 2-(4-(bis(benzyloxy)phosphoryl)phenyl)-2-((tert-butoxycarbonyl)amino)acetic acid (500 mg, 0.98 mmol) in DCM (4.0 mL) at 0° C. was added TFA (1.0 mL). The solution was warmed to RT for 2 h then concentrated.

### Step 3b.

The crude product was dissolved in THF (4.0 mL)/NaHCO₃ (sat. aq., 4.0 mL) followed by addition of 4-(2-chloro-3,4-dimethoxybenzyl)-2,3-dioxopiperazine-1-carbonyl chloride (388 mg, 1.07 mmol, 1.1 equiv.). The reaction was stirred at RT for 2 h. The reaction was diluted with H₂O (5 mL) and acidified until pH 2 with HCl (2 M). Ethyl acetate (50 mL) was added and the organic layer was washed with H₂O (2×10 mL) and brine (1×10 mL), dried (Na₂SO₄), and concentrated. The crude product was purified by silica gel chromatography (0-20% MeOH/DCM) to yield title compound (375 mg, 69% over 2 steps). ESI-MS m/z 737 (M+H)⁺.

### Step 4. Synthesis of (3R)-3-(2-(4-(2-chloro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

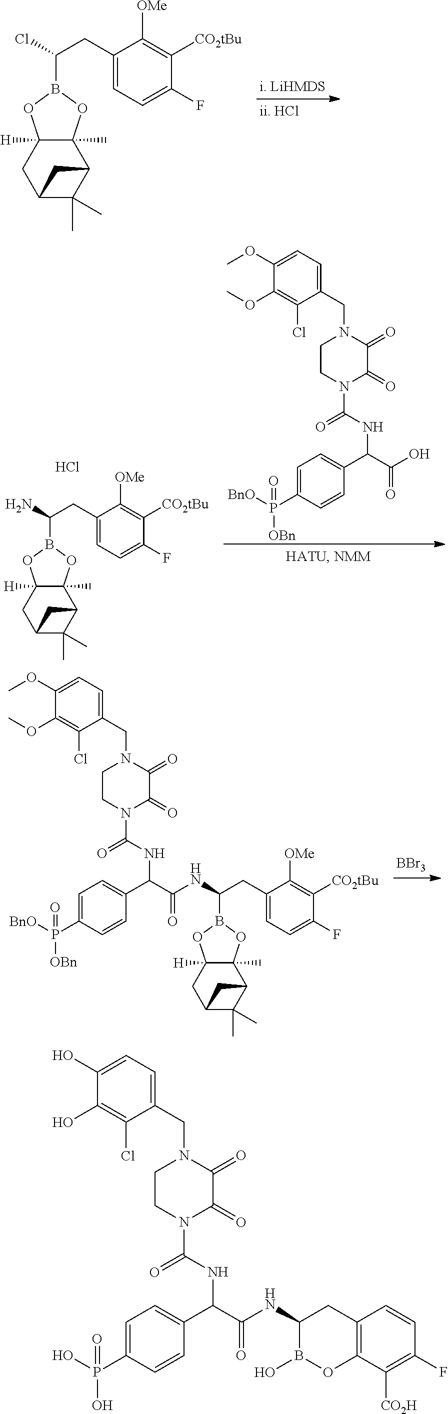

### Step 4a.

A solution of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (5.2 g, 11 mmol) (Example 200, Step 7) in THF (45 mL) was cooled to −78° C. LiHMDS (1 M in THF, 11 mL, 11 mmol, 1.0 equiv.) was added dropwise and the mixture was warmed to RT for 1 h. The solution was cooled to 0° C. and HCl (4 M in dioxane, 11 mL, 44 mmol, 4.0 eq) was added dropwise. The reaction was warmed to RT for 1 h then concentrated. Hexanes (200 mL) was added and stirred at RT overnight. The solid was filtered, washed with hexanes (2×100 mL), and dried to yield desired intermediate tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (5.8 g, 99%).

### Step 4b.

To a solution of tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (198 mg, 0.340 mmol), 2-(4-(bis(benzyloxy)phosphoryl)phenyl)-2-(4-(2-chloro-3,4-dimethoxybenzyl)-2,3-dioxopiperazine-1-carboxamido)acetic acid (375 mg, 0.509 mmol, 1.5 equiv.), and HATU (226 mg, 0.595 mmol, 1.75 equiv.) in DMA (3.4 mL) was added NMM (0.11 mL, 1.02 mmol, 3.0 equiv.). The reaction was stirred at RT for 30 min. and diluted with EtOAc (30 mL). The mixture was washed with H₂O (2×10 mL) and brine (xx mL), dried (Na₂SO₄), filtered, and concentrated. The crude product was purified by silica gel chromatography (0-5% MeOH/DCM) to yield desired tert-butyl 3-((2R)-2-(2-(4-(bis(benzyloxy)phosphoryl)phenyl)-2-(4-(2-chloro-3,4-dimethoxybenzyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (252 mg, 64%) ESI m z 1166 (M+H)⁺.

### Step 4c.

To a solution of tert-butyl 3-((2R)-2-(2-(4-(bis(benzyloxy)phosphoryl)phenyl)-2-(4-(2-chloro-3,4-dimethoxybenzyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (252 mg, 0.216 mmol) in DCM (2.2 mL) at −78° C. was added BBr₃ (1 M in DCM, 2.2 mL, 2.2 mmol, 10 equiv.). The reaction was warmed to RT overnight then concentrated. The resulting crude product was purified by reverse-phase HPLC to yield (3R)-3-(2-(4-(2-chloro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid (44 mg, 28%). ESI m/z 735 (M+H)⁺.

### Example 9: Synthesis of (R)-3-((S)-2-(4-ethyl-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 1, utilizing tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in Step 2. After reversed phase HPLC purification in Step 5, the title compound was collected as the first eluting peak. ESI-MS m/z 607 (M+H)⁺.

### Example 10: Synthesis of (R)-3-((R)-2-(4-ethyl-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 9 and collected as the second eluting peak after reversed phase HPLC purification. ESI-MS m/z 607 (M+H)⁺.

### Example 11: Synthesis of (3R)-3-(2-(3-chloro-4-phosphonophenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 1, utilizing 3-chloro-4-iodobenzaldehyde in place of 2-fluoro-4-iodobenzaldehyde in Step 1 and Xantphos Pd G3 in place of tetrakis(triphenylphosphine)palladium(0) in Step 4. ESI-MS 645 m/z (M+H)⁺.

### Example 12: Synthesis of (3R)-3-(2-(3-fluoro-4-phosphonophenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 1, utilizing 3-fluoro-4-iodobenzaldehyde in place of 2-fluoro-4-iodobenzaldehyde in Step 1 and Xantphos Pd G3 in place of tetrakis(triphenylphosphine)palladium(0) in Step 4. ESI-MS 629 m/z (M+H)⁺.

### Example 13: Synthesis of (R)-3-((R)-2-(2,3-difluoro-4-phosphonophenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 1, utilizing 2,3-difluoro-4-iodobenzaldehyde in place of 2-fluoro-4-iodobenzaldehyde in Step 1 and Xantphos Pd G3 in place of tetrakis(triphenylphosphine)palladium(0) in Step 4. After reversed phase HPLC purification in Step 5, the title compound was collected as the second eluting peak ESI-MS 647 m z (M+H)⁺.

### Example 14: (3R)-3-(2-amino-2-(4-phosphonophenyl)acetamido)-7-fluoro-2-hydroxy-3,4-dihydro-2H-benzo[e][1,2]oxaborinine-8-carboxylic acid

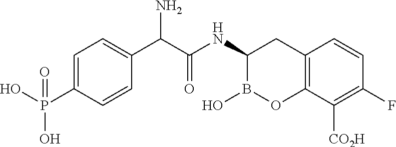 [SMILES: NC(C(=O)N[C@H]1Cc2ccc(F)c(C(=O)O)c2OB1O)c1ccc(P(=O)(O)O)cc1]

The title compound was prepared in a similar manner to the synthesis of Example 8, utilizing 2-(4-(bis(benzyloxy)phosphoryl)phenyl)-2-((tert-butoxycarbonyl)amino)acetic acid in place of 2-(4-(bis(benzyloxy)phosphoryl)phenyl)-2-(4-(2-chloro-3,4-dimethoxybenzyl)-2,3-dioxopiperazine-1-carboxamido)acetic acid in Step 4b. The title compound was isolated by reverse-phase HPLC. ESI-MS m/z 438 (M+H)⁺.

### Example 15: Synthesis of (R)-3-((R)-2-(3-fluoro-4-phosphonophenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 12 and collected as the second eluting peak after reversed phase HPLC purification. ESI-MS m/z 629 (M+H)⁺.

### Example 16: Synthesis of (R)-3-((R)-2-(2,3-difluoro-4-phosphonophenyl)-2-(3-(methylsulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 13, utilizing tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate (Step 2 in Example 1). ESI-MS m/z 665 (M+H)⁺.

### Example 17: Synthesis of (3R)-2-hydroxy-3-(2-(3-hydroxy-4-phosphonophenyl)-2-(3-(methylsulfonyl)-2-oxoimidazolidine-1-carboxamido)acetamido)-3,4-dihydro-2H-benzo[e][1,2]oxaborinine-8-carboxylic acid

The title compound was prepared in a similar manner to the synthesis of Example 1, utilizing 3-(benzyloxy)-4-iodobenzaldehyde in place of 2-fluoro-4-iodobenzaldehyde in Step 1 and Xantphos Pd G3 in place of tetrakis(triphenylphosphine)palladium(0) in Step 4. ESI-MS m/z 627 (M+H)⁺.

### Example 18: Synthesis of (R)-3-((R)-2-(3,5-difluoro-4-phosphonophenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 1, utilizing 3,5-difluoro-4-iodobenzaldehyde in place of 2-fluoro-4-iodobenzaldehyde in Step 1 and Xantphos Pd G3 in place of tetrakis(triphenylphosphine)palladium(0) in Step 4. After reversed phase HPLC purification in Step 5, the title compound was collected as the second eluting peak ESI-MS m/z 647 (M+H)⁺

### Example 19: Synthesis of (R)-7-fluoro-3-((R)-2-(3-fluoro-4-phosphonophenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 9 utilizing 3-fluoro-4-iodobenzaldehyde in place of 2-fluoro-4-iodobenzaldehyde (Step 1 in Example 1) and collected as the second eluting peak after reversed phase HPLC purification. ESI-MS m/z 647 (M+H)⁺.

### Example 20: Synthesis of (3R)-3-(2-(4-ethyl-2,3-dioxopiperazine-1-carboxamido)-2-(4-(phosphonomethyl)phenyl)acetamido)-2-hydroxy-3,4-dihydro-2H-benzo[e][1,2]oxaborinine-8-carboxylic acid

### Step 1: Synthesis of (4-(2-(((R)-2-(3-(tert-butoxycarbonyl)-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(4-ethyl-2,3-dioxopiperazine-1-carboxamido)-2-oxoethyl)benzyl)phosphonic acid

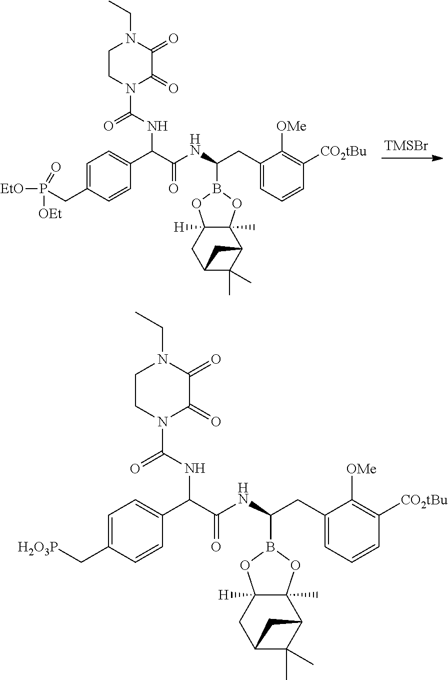

To tert-butyl 3-((2R)-2-(2-(4-((diethoxyphosphoryl)methyl)phenyl)-2-(4-ethyl-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate 0.44 g (0.54 mmol) in dichloromethane (12 mL) at 0° C. was add trimethylsilyl bromide 0.21 mL (1.61 mmol, 3 eq) drop wise, warmed at RT for 18 h, and concentrated in vacuo to give the title compound. ESI-MS m/z 769 (M+H)⁺.

### Step 2: Synthesis of (3R)-3-(2-(4-ethyl-2,3-dioxopiperazine-1-carboxamido)-2-(4-(phosphonomethyl)phenyl)acetamido)-2-hydroxy-3,4-dihydro-2H-benzo[e][1,2]oxaborinine-8-carboxylic acid

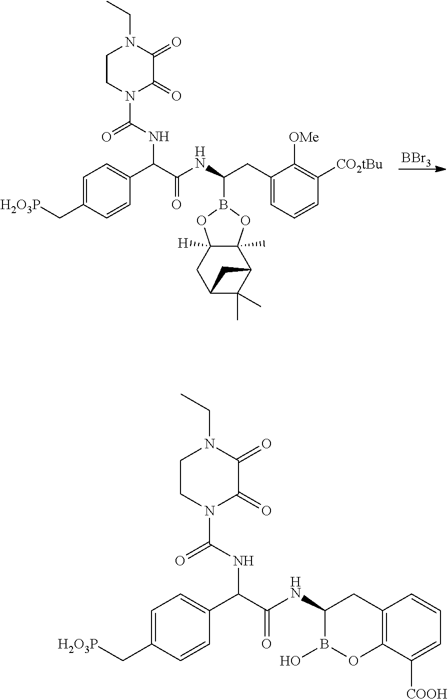

The title compound was prepared by following the general deprotection and purification method in Step 5 of Example 1. ESI-MS m/z 603 (M+H)⁺.

### Example 21: Synthesis of (3R)-3-(2-(4-ethyl-2,3-dioxopiperazine-1-carboxamido)-2-(3-hydroxy-4-phosphonophenyl)acetamido)-2-hydroxy-3,4-dihydro-2H-benzo[e][1,2]oxaborinine-8-carboxylic acid

The title compound was prepared in a similar manner to the synthesis of Example 17, utilizing 4-ethyl-2,3-dioxopiperazine-1-carbonyl chloride in place of 3-(methylsulfonyl)-2-oxoimidazolidine-1-carbonyl chloride in (Example 1, Step 3). ESI-MS m/z 605 (M+H)⁺.

### Example 22: Synthesis of (3R)-3-(2-(4-ethyl-2,3-dioxopiperazine-1-carboxamido)-2-(3-(phosphonomethyl)phenyl)acetamido)-2-hydroxy-3,4-dihydro-2H-benzo[e][1,2]oxaborinine-8-carboxylic acid

The title compound was prepared in a similar manner to the synthesis of Example 20, utilizing diethyl (3-formylbenzyl)phosphonate in place of diethyl (4-formylbenzyl)phosphonate (Example 1, Step 1). ESI-MS m/z 603 (M+H)⁺.

### Example 23: Synthesis of (3R)-3-(2-(4-(2-chloro-3,4-dihydroxybenzyl)-2,3-dioxopiperazine-1-carboxamido)-2-(3-fluoro-4-phosphonophenyl)acetamido)-7-fluoro-2-hydroxy-3,4-dihydro-2H-benzo[e][1,2]oxaborinine-8-carboxylic acid

The title compound was synthesized in a similar manner as Example 8, utilizing 3-fluoro-4-iodobenzaldehyde in place of 4-iodobenzaldehyde (Example 8, Step 1). ESI m/z 753 (M+H)⁺.

### Example 24: Synthesis of (3R)-3-(2-(4-(2-chloro-3,4-dihydroxybenzyl)-2,3-dioxopiperazine-1-carboxamido)-2-(4-phosphonophenyl)acetamido)-2-hydroxy-3,4-dihydro-2H-benzo[e][1,2]oxaborinine-8-carboxylic acid

The title compound was synthesized in a similar manner as Example 8, utilizing tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate (WO 2014/089365) in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (Example 8, Step 4). ESI m/z 717 (M+H)⁺.

### Example 25: Synthesis of (3R)-3-(2-(2,6-difluoro-4-phosphonophenyl)-2-(4-ethyl-2,3-dioxopiperazine-1-carboxamido)acetamido)-2-hydroxy-3,4-dihydro-2H-benzo[e][1,2]oxaborinine-8-carboxylic acid

The title compound was prepared in a similar manner to the synthesis of Example 1, utilizing 2,6-difluoro-4-iodobenzaldehyde in place of 2-fluoro-4-iodobenzaldehyde in Step 1 and Xantphos Pd G3 in place of tetrakis(triphenylphosphine)palladium(0) in Step 4. ESI-MS m/z 625 (M+H)⁺.

### Example 26: Synthesis of (3R)-3-(2-acetamido-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing acetyl chloride in place of 4-ethyl-2,3-dioxopiperazine-1-carbonyl chloride to give the title compound. ESI-MS m/z 463 (M+H)⁺.

### Example 27: Synthesis of (3R)-2-hydroxy-3-(2-(4-phosphonophenyl)-2-propionamidoacetamido)-3,4-dihydro-2H-benzo[e][1,2]oxaborinine-8-carboxylic acid

The title compound was prepared in a similar manner to the synthesis of Example 1, utilizing propionyl chloride in place of 4-ethyl-2,3-dioxopiperazine-1-carbonyl chloride to give the title compound. ESI-MS m/z 477 (M+H)⁺.

### Example 28: Synthesis of (3R)-2-hydroxy-3-(2-isobutyramido-2-(4-phosphonophenyl)acetamido)-3,4-dihydro-2H-benzo[e][1,2]oxaborinine-8-carboxylic acid

The title compound was prepared in a similar manner to the synthesis of Example 1, utilizing isobutyryl chloride in place of 4-ethyl-2,3-dioxopiperazine-1-carbonyl chloride to give the title compound. ESI-MS m/z 491 (M+H)⁺.

### Example 29: Synthesis of (3R)-2-hydroxy-3-(2-(4-phosphonophenyl)-2-pivalamidoacetamido)-3,4-dihydro-2H-benzo[e][1,2]oxaborinine-8-carboxylic acid

The title compound was prepared in a similar manner to the synthesis of Example 1, utilizing pivaloyl chloride in place of 4-ethyl-2,3-dioxopiperazine-1-carbonyl chloride to give the title compound. ESI-MS m/z 505 (M+H)⁺.

### Example 30: Synthesis of (3R)-3-(2-(cyclopropanecarboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing cyclopropanecarbonyl chloride in place of 4-ethyl-2,3-dioxopiperazine-1-carbonyl chloride to give the title compound. ESI-MS m/z 489 (M+H)⁺.

### Example 31: Synthesis of (3R)-3-(2-(cyclohexanecarboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing cyclohexanecarbonyl chloride in place of 4-ethyl-2,3-dioxopiperazine-1-carbonyl chloride to give the title compound. ESI-MS m/z 531 (M+H)⁺.

### Example 32: Synthesis of (3R)-2-hydroxy-3-(2-(4-phosphonophenyl)-2-(picolinamido)acetamido)-3,4-dihydro-2H-benzo[e][1,2]oxaborinine-8-carboxylic acid

The title compound was prepared in a similar manner to the synthesis of Example 1, utilizing picolinoyl chloride in place of 4-ethyl-2,3-dioxopiperazine-1-carbonyl chloride to give the title compound. ESI-MS m/z 526 (M+H)⁺.

### Example 33: Synthesis of (3R)-3-(2-((R)-1-acetylpyrrolidine-2-carboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing acetyl-L-prolinoyl chloride in place of 4-ethyl-2,3-dioxopiperazine-1-carbonyl chloride to give the title compound. ESI-MS m/z 560 (M+H)⁺.

### Example 34: (Synthesis of R)-2-hydroxy-3-((S)-2-((R)-1-(methylsulfonyl)pyrrolidine-2-carboxamido)-2-(4-phosphonophenyl)acetamido)-3,4-dihydro-2H-benzo[e][1,2]oxaborinine-8-carboxylic acid and Example 35: Synthesis of (R)-2-hydroxy-3-((R)-2-((R)-1-(methylsulfonyl)pyrrolidine-2-carboxamido)-2-(4-phosphonophenyl)acetamido)-3,4-dihydro-2H-benzo[e][1,2]oxaborinine-8-carboxylic acid

The title compounds were prepared in a similar manner to the synthesis of Example 1, utilizing (methylsulfonyl)-D-prolinoyl chloride in place of 4-ethyl-2,3-dioxopiperazine-1-carbonyl chloride to give the title compounds after purification by HPLC. ESI-MS m/z 596 (M+H)⁺.

### Example 36: Synthesis of (R)-2-hydroxy-3-((S)-2-(2-oxopiperidine-1-carboxamido)-2-(4-phosphonophenyl)acetamido)-3,4-dihydro-2H-benzo[39e][1,2]oxaborinine-8-carboxylic acid and EXAMPLE 37: Synthesis of (R)-2-hydroxy-3-((R)-2-(2-oxopiperidine-1-carboxamido)-2-(4-phosphonophenyl)acetamido)-3,4-dihydro-2H-benzo[e][1,2]oxaborinine-8-carboxylic acid

The title compounds were prepared in a similar manner to the synthesis of Example 1, utilizing 2-oxopiperidine-1-carbonyl chloride in place of 4-ethyl-2,3-dioxopiperazine-1-carbonyl chloride to give the title compounds after purification by HPLC. ESI-MS m/z 546 (M+H)⁺.

### Example 38: Synthesis of (3R)-2-hydroxy-3-(2-(3-methyl-2-oxohexahydropyrimidine-1-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 1, utilizing 3-methyl-2-oxotetrahydropyrimidine-1(2H)-carbonyl chloride in place of 4-ethyl-2,3-dioxopiperazine-1-carbonyl chloride to give the title compound. ESI-MS m/z 561 (M+H)⁺.

### Example 39: Synthesis of (R)-2-hydroxy-3-((S)-2-(2-oxoimidazolidine-1-carboxamido)-2-(4-phosphonophenyl)acetamido)-3,4-dihydro-2H-benzo[e][1,2]oxaborinine-8-carboxylic acid and

### Example 40: Synthesis of (R)-2-hydroxy-3-((R)-2-(2-oxoimidazolidine-1-carboxamido)-2-(4-phosphonophenyl)acetamido)-3,4-dihydro-2H-benzo[e][1,2]oxaborinine-8-carboxylic acid

The title compounds were prepared in a similar manner to the synthesis of Example 1, utilizing 2-oxoimidazolidine-1-carbonyl chloride in place of 4-ethyl-2,3-dioxopiperazine-1-carbonyl chloride to give the title compounds after purification by HPLC. ESI-MS m/z 533 (M+H)⁺.

### Example 41: Synthesis of (3R)-2-hydroxy-3-(2-(3-methyl-2-oxoimidazolidine-1-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 1, utilizing 3-methyl-2-oxoimidazolidine-1-carbonyl chloride in place of 4-ethyl-2,3-dioxopiperazine-1-carbonyl chloride to give the title compound. ESI-MS m/z 547 (M+H)⁺.

### Example 42: Synthesis of (3R)-3-(2-(3-ethyl-2-oxoimidazolidine-1-carboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing 3-ethyl-2-oxoimidazolidine-1-carbonyl chloride in place of 4-ethyl-2,3-dioxopiperazine-1-carbonyl chloride to give the title compound. ESI-MS m/z 561 (M+H)⁺.

### Example 43: Synthesis of (R)-2-hydroxy-3-((S)-2-((S)-1-(methylsulfonyl)pyrrolidine-2-carboxamido)-2-(4-phosphonophenyl)acetamido)-3,4-dihydro-2H-benzo[e][1,2]oxaborinine-8-carboxylic acid and EXAMPLE 44: Synthesis of (R)-2-hydroxy-3-((R)-2-((S)-1-(methylsulfonyl)pyrrolidine-2-carboxamido)-2-(4-phosphonophenyl)acetamido)-3,4-dihydro-2H-benzo[e][1,2]oxaborinine-8-carboxylic acid

The title compounds were prepared in a similar manner to the synthesis of Example 1, utilizing (methylsulfonyl)-L-prolinoyl chloride in place of 4-ethyl-2,3-dioxopiperazine-1-carbonyl chloride to give the title compounds after purification by HPLC. ESI-MS m/z 596 (M+H)⁺.

### Example 45: Synthesis of (3R)-3-(2-(4-(2-chloro-5-fluoro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 2-chloro-5-fluoro-3,4-dimethoxybenzaldehyde

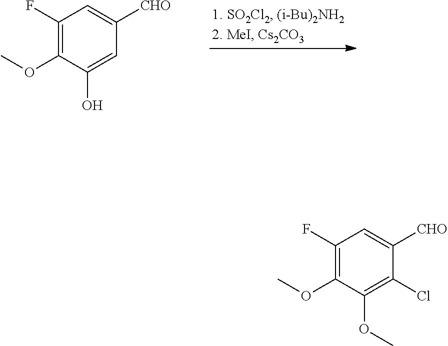

### Step 1a.

A solution of 3-fluoro-5-hydroxy-4-methoxybenzaldehyde (8.00 g, 47.0 mmol) in toluene (170 mL) was cooled to 0° C. (i-Bu)₂NH (4.00 mL, 49.4 mmol, 1.05 equiv.) was added followed by SO₂Cl₂ (0.82 mL, 4.70 mmol, 0.1 equiv.) slowly. The mixture was heated to 70° C. overnight then cooled to RT and quenched with H₂O (50 mL). The layers were separated and the aq. layer was extracted with EtOAc (3×50 mL). The combined organic layers were dried (Na₂SO₄), filtered, and concentrated.

### Step 1b.

The crude product was dissolved in DMF (120 mL) and Cs₂CO₃ (38.3 g, 117 mmol, 2.5 equiv.) was added followed by MeI (8.8 mL, 140 mmol, 3.0 equiv.). The mixture was stirred at RT for 1 h, diluted with EtOAc (300 mL), and quenched with H₂O (150 mL). The layers were separated and the aq. layer was extracted with EtOAc (3×150 mL). The combined organic layers were washed with H₂O (3×75 mL) and brine (75 mL), dried (Na₂SO₄), filtered, and concentrated. The crude product was purified by silica gel chromatography (0-20%, EtOAc/Hexanes). ESI m/z 219 (M+H)⁺.

### Step 2. Synthesis of (3R)-3-(2-(4-(2-chloro-5-fluoro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was synthesized in a similar manner as Example 8, utilizing 2-chloro-5-fluoro-3,4-dimethoxybenzaldehyde in place of 2-chloro-3,4-dimethoxybenzaldehyde (Example 8, Step 2). ESI m/z 753 (M+H)⁺.

### Example 46: Synthesis of (3R)-3-(2-(3-(2-chloro-3,4-dihydroxybenzyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 3-(2-chloro-3,4-dimethoxybenzyl)-2-oxoimidazolidine-1-carbonyl chloride

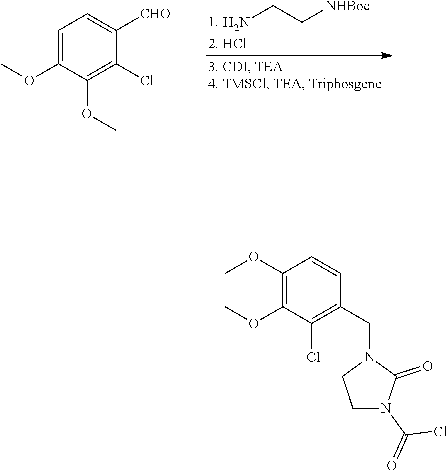 [SMILES: COc1ccc(C=O)c(Cl)c1OC ; CC(C)(C)OC(=O)NCCN ; COc1ccc(CN2CCN(C(=O)Cl)C2=O)c(Cl)c1OC]

### Step 1a.

The reaction was performed in a similar manner as Example 8, Step 2a.

### Step 1b.

The reaction was performed in a similar manner as Example 8, Step 2b.

### Step 1c.

To a mixture of crude product (14.95 mmol) in THF (150 mL) was added TEA (6.3 mL, 45 mmol, 3.0 equiv.) and CDI (2.7 g. 16 mmol, 1.1 equiv.). The reaction was stirred at RT overnight then concentrated. The crude product was purified by silica gel chromatography (70-100% EtOAc/Hexanes) to provide desired intermediate (2.59 g, 64% over 3 steps). ESI m/z 271 (M+H)⁺.

### Step 1d.

The reaction was performed in a similar manner as Example 8, Step 2d.

### Step 2. Synthesis of (3R)-3-(2-(3-(2-chloro-3,4-dihydroxybenzyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was synthesized in a similar manner as Example 8, utilizing 3-(2-chloro-3,4-dimethoxybenzyl)-2-oxoimidazolidine-1-carbonyl chloride in place of 4-(2-chloro-3,4-dimethoxybenzyl)-2,3-dioxopiperazine-1-carbonyl chloride (Example 8, Step 2). ESI m/z 707 (M+H)⁺.

### Example 47: Synthesis of (3R)-3-((2R)-2-(4-ethyl-2,3-dioxopiperazine-1-carboxamido)-2-(4-(hydroxy(methyl)phosphoryl)phenyl)acetamido)-2-hydroxy-3,4-dihydro-2H-benzo[e][1,2]oxaborinine-8-carboxylic acid

The title compound was prepared in a similar manner to the synthesis of Example 1, utilizing ethyl methylphosphinate in place of dibenzyl phosphite in Step 4. After reversed phase HPLC purification in Step 5, the title compound was collected as the second eluting peak. ESI-MS m/z 587 (M+H)⁺.

### Example 48: Synthesis of (3R)-3-(2-(3-fluoro-2-hydroxy-4-phosphonophenyl)-2-(3-(methylsulfonyl)-2-oxoimidazolidine-1-carboxamido)acetamido)-2-hydroxy-3,4-dihydro-2H-benzo[e][1,2]oxaborinine-8-carboxylic acid

The title compound was prepared in a similar manner to the synthesis of Example 1, utilizing 2-(benzyloxy)-3-fluoro-4-iodobenzaldehyde in place of 2-fluoro-4-iodobenzaldehyde in Step 1 and Xantphos Pd G3 in place of tetrakis(triphenylphosphine)palladium(0) in Step 4. ESI-MS m/z 645 (M+H)⁺.

### Example 49: Synthesis of (R)-3-((R)-2-(2-chloro-3,4-dihydroxybenzamido)-2-(4-phosphonophenyl)acetamido)-2-hydroxy-3,4-dihydro-2H-benzo[e][1,2]oxaborinine-8-carboxylic acid

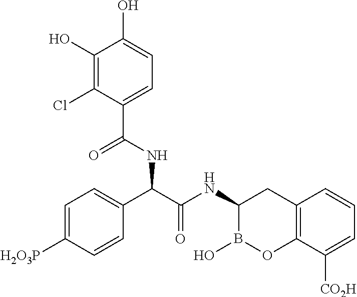

### Step 1. Synthesis of 2-(4-(bis(benzyloxy)phosphoryl)phenyl)-2-((tert-butoxycarbonyl)amino)acetic acid

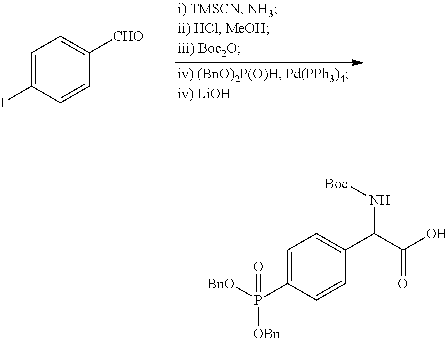 [SMILES: O=Cc1ccc(I)cc1 ; CC(C)(C)OC(=O)NC(C(=O)O)c1ccc(P(=O)(OCc2ccccc2)OCc2ccccc2)cc1]

### Step 1a.

To 4-iodobenzaldehyde (46.2 g, 200 mmol) at 0° C. was added 7 N ammonia in methanol (600 mL), followed by trimethylsilyl cyanide (29.8 g, 300 mmol, 1.5 eq). The mixture was stirred at 45° C. for 24 h and concentrated in vacuo.

### Step 1b.

The crude product was dissolved in 3 N hydrochloric acid in methanol (400 mL), stirred at 50° C. for 36 h and concentrated in vacuo.

### Step 1c.

To the crude product in DCM (400 mL) at 0° C. was added triethylamine (30.3 g, 300 mmol, 3 eq) followed by di-tert-butyl dicarbonate (65.4 g, 300 mmol, 1.5 eq). The reaction was warmed to RT for 12 h and concentrated in vacuo. The crude product was purified by flash silica gel chromatography (10% ethyl acetate/hexanes) to give the desired intermediate (51 g, 66% for 3 steps).

### Step 1d.

To methyl 2-((tert-butoxycarbonyl)amino)-2-(4-iodophenyl)acetate (20 g, 52 mmol) was added N,N-diisopropylethylamine 20.2 g, 156 mmol, 3 eq), Pd(PPh₃)₄ (11.8 g, 10.2 mmol, 20 mol %), dibenzyl phosphite (26.7 g, 102 mmol, 2 eq), followed by anhydrous toluene (400 mL) and the mixture was stirred at RT for 5-7 h under argon. The reaction was diluted with ethyl acetate, washed with water then brine, dried over sodium sulfate, and concentrated. The product was purified by flash chromatography on silica gel (35-50% ethyl acetate/hexanes) to give the desired intermediate (27 g, 99%).

### Step 1e.

To methyl 2-(4-(bis(benzyloxy)phosphoryl)phenyl)-2-((tert-butoxycarbonyl)amino)acetate (27 g, 51.4 mmol) in THF (100 mL)/H₂O (100 mL) was added lithium hydroxide monohydrate (6.48 g, 154 mmol, 3 eq) and the mixture was stirred at RT for 1 h. 2N Hydrochloric acid was added drop wise to obtain pH 2 and extracted with dichloromethane. The organic layer was washed with water, dried over sodium sulfate, and concentrated to give 20 g crude product. The product was purified by flash chromatography on silica gel (10-20% MeOH/DCM) to give the title compound (15.8 g, 60%) as a white solid. ESI-MS m/z 512 (M+H)⁺.

### Step 2: Synthesis of tert-butyl 3-((2R)-2-(2-(4-(bis(benzyloxy)phosphoryl)phenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-7-yl)ethyl)-7-methoxybenzoate

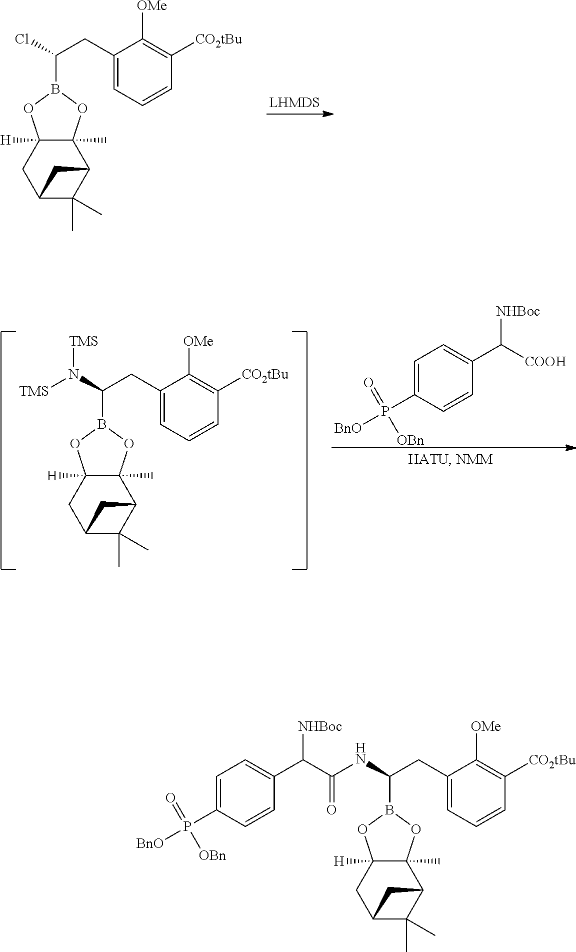

By following the General procedure C, the chloride (prepared as previous reported, WO 2014/089365) was treated with LiHMDS, and then coupled with 2-(4-(bis(benzyloxy)phosphoryl)phenyl)-2-((tert-butoxycarbonyl)amino)acetic acid in the presence of HATU and NMM, yielding the title compound. ESI-MS m/z 923 (M+H)⁺.

### Step 3: (R)-3-((R)-2-(2-chloro-3,4-dihydroxybenzamido)-2-(4-phosphonophenyl)acetamido)-2-hydroxy-3,4-dihydro-2H-benzo[e][1,2]oxaborinine-8-carboxylic acid

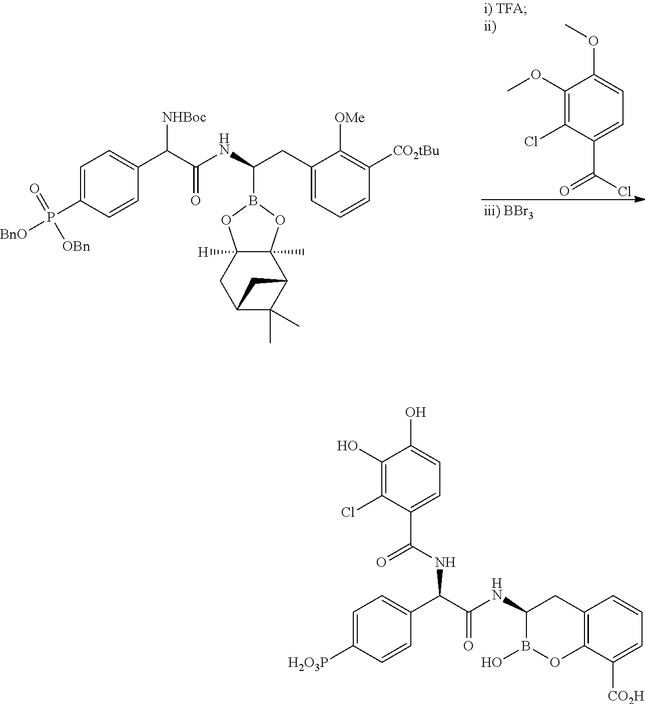

i) tert-Butyl 3-((2R)-2-(2-(4-(bis(benzyloxy)phosphoryl)phenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate at 0° C. was added 1 N trifluoroacetic acid in dichloromethane and warmed at RT for 6 h. The reaction was concentrated in vacuo to give 3-((2R)-2-(2-amino-2-(4-(bis(benzyloxy)phosphoryl)phenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoic acid-2,2,2-trifluoro-113-ethan-1-one (1/1). ESI-MS m/z 767 (M+H)⁺.

ii) To above crude in dichloromethane at 0° C. was added N,N-diisopropylethylamine, followed by 2-chloro-3,4-dimethoxybenzoyl chloride and the reaction was warmed at RT for 30 min. The product was quenched with water, washed with brine, dried over sodium sulfate, and concentrated to give 3-((2R)-2-(2-(4-(bis(benzyloxy)phosphoryl)phenyl)-2-(2-chloro-3,4-dimethoxybenzamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoic acid taken directly on to the next step without further purification. ESI-MS m/z 965 (M+H)⁺.

iii) To 3-((2R)-2-(2-(4-(bis(benzyloxy)phosphoryl)phenyl)-2-(2-chloro-3,4-dimethoxybenzamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoic acid in dichloromethane at −78° C. was added 1M boron tribromide in dichloromethane drop wise and warmed at RT for 30 min. The crude mixture was cooled at 0° C., quenched with H₂O/MeOH, and concentrated. The title compound was purified by reversed phase HPLC. ESI-MS m/z 591 (M+H)⁺.

### Example 50: Synthesis of (R)-3-((S)-2-(2-aminothiazole-5-carboxamido)-2-(4-phosphonophenyl)acetamido)-2-hydroxy-3,4-dihydro-2H-benzo[e][1,2]oxaborinine-8-carboxylic acid and (R)-3-((R)-2-(2-aminothiazole-5-carboxamido)-2-(4-phosphonophenyl)acetamido)-2-hydroxy-3,4-dihydro-2H-benzo[e][1,2]oxaborinine-8-carboxylic acid

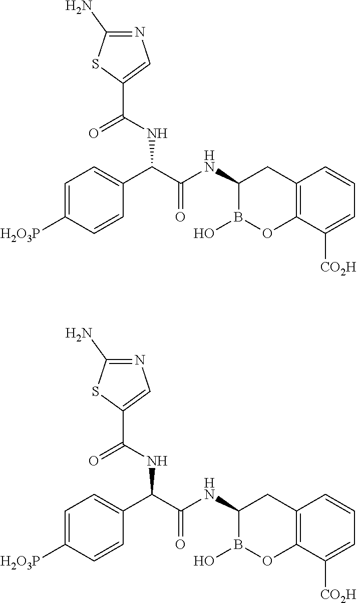

The title compounds were prepared in a similar manner to the synthesis of Example 49, utilizing tert-butyl (5-(chlorocarbonyl)thiazol-2-yl)carbamate in place of 2-chloro-3,4-dimethoxybenzoyl chloride in Step 3. ESI-MS m/z 547 (M+H)⁺.

### Example 51: Synthesis of (R)-3-((S)-2-(2-aminothiazole-4-carboxamido)-2-(4-phosphonophenyl)acetamido)-2-hydroxy-3,4-dihydro-2H-benzo[e][1,2]oxaborinine-8-carboxylic acid and (R)-3-((R)-2-(2-aminothiazole-4-carboxamido)-2-(4-phosphonophenyl)acetamido)-2-hydroxy-3,4-dihydro-2H-benzo[e][1,2]oxaborinine-8-carboxylic acid

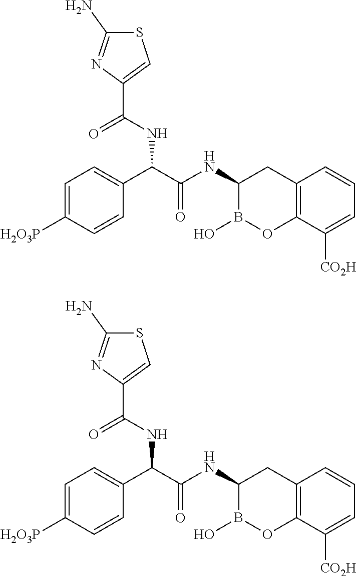

The title compounds were prepared in a similar manner to Example 49, utilizing tert-butyl (4-(chlorocarbonyl)thiazol-2-yl)carbamate in place of 2-chloro-3,4-dimethoxybenzoyl chloride in Step 3. ESI-MS m/z 547 (M+H)⁺.

### Example 52: Synthesis of (3R)-2-hydroxy-3-(2-(4-phosphonophenyl)-2-((R)-pyrrolidine-2-carboxamido)acetamido)-3,4-dihydro-2H-benzo[e][1,2]oxaborinine-8-carboxylic acid

The title compound was prepared in a similar manner to the synthesis of Example 49, utilizing D-prolinoyl chloride in place of 2-chloro-3,4-dimethoxybenzoyl chloride in Step 3. ESI-MS m/z 518 (M+H)⁺.

### Example 53: Synthesis of (3R)-2-hydroxy-3-(2-((R)-5-oxopyrrolidine-2-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing (R)-5-oxopyrrolidine-2-carbonyl chloride in place of 2-chloro-3,4-dimethoxybenzoyl chloride in Step 3. ESI-MS m/z 532 (M+H)⁺.

### Example 54: Synthesis of (R)-2-hydroxy-3-((S)-2-((R)-1-methylpyrrolidine-2-carboxamido)-2-(4-phosphonophenyl)acetamido)-3,4-dihydro-2H-benzo[e][1,2]oxaborinine-8-carboxylic acid and (R)-2-hydroxy-3-((R)-2-((R)-1-methylpyrrolidine-2-carboxamido)-2-(4-phosphonophenyl)acetamido)-3,4-dihydro-2H-benzo[e][1,2]oxaborinine-8-carboxylic acid

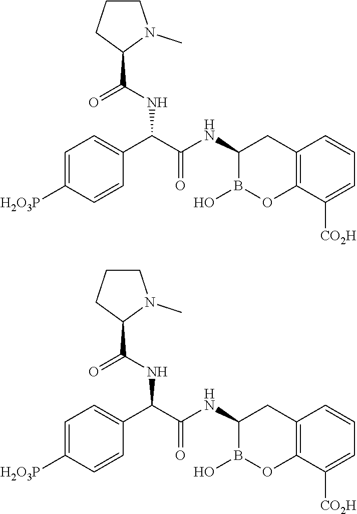

The title compounds were prepared in a similar manner to the synthesis of Example 49, utilizing methyl-D-prolinoyl chloride in place of 2-chloro-3,4-dimethoxybenzoyl chloride in Step 3. ESI-MS m/z 532 (M+H)⁺.

### Example 56: Synthesis of (3R)-2-hydroxy-3-(2-(2-oxohexahydropyrimidine-1-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing tert-butyl 3-(chlorocarbonyl)-2-oxotetrahydropyrimidine-1(2H)-carboxylate in place of 2-chloro-3,4-dimethoxybenzoyl chloride in Step 3. ESI-MS m/z 547 (M+H)⁺.

### Example 57: Synthesis of (3R)-2-hydroxy-3-(2-(2-oxopyrrolidine-1-carboxamido)-2-(4-phosphonophenyl)acetamido)-3,4-dihydro-2H-benzo[e][1,2]oxaborinine-8-carboxylic acid

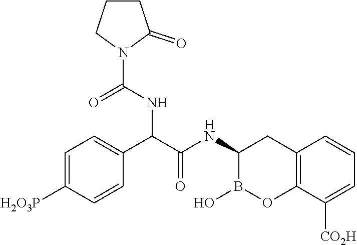

The title compound was prepared in a similar manner to the synthesis of Example 49, utilizing 2-oxopyrrolidine-1-carbonyl chloride in place of 2-chloro-3,4-dimethoxybenzoyl chloride in Step 3. ESI-MS m/z 532 (M+H)⁺.

### Example 58: Synthesis of (3R)-2-hydroxy-3-(2-(4-phosphonophenyl)-2-(sulfamoylamino)acetamido)-3,4-dihydro-2H-benzo[e][1,2]oxaborinine-8-carboxylic acid

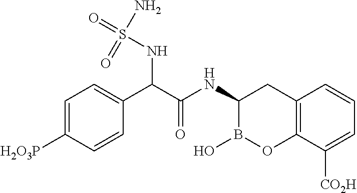

The title compound was prepared in a similar manner to the synthesis of Example 49, utilizing tert-butyl (chlorosulfonyl)carbamate in place of 2-chloro-3,4-dimethoxybenzoyl chloride in Step 3. ESI-MS m/z 500 (M+H)⁺.

### Example 59: Synthesis of (3R)-2-hydroxy-3-(2-(methylsulfonamido)-2-(4-phosphonophenyl)acetamido)-3,4-dihydro-2H-benzo[e][1,2]oxaborinine-8-carboxylic acid

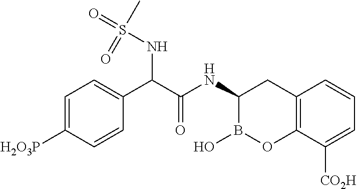

The title compound was prepared in a similar manner to the synthesis of Example 49, utilizing methanesulfonyl chloride in place of 2-chloro-3,4-dimethoxybenzoyl chloride in Step 3. ESI-MS m/z 499 (M+H)⁺.

### Example 60: Synthesis of (3R)-2-hydroxy-3-(2-((1-methylethyl)sulfonamido)-2-(4-phosphonophenyl)acetamido)-3,4-dihydro-2H-benzo[e][1,2]oxaborinine-8-carboxylic acid

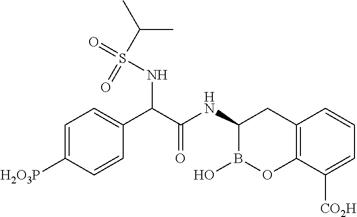

The title compound was prepared in a similar manner to the synthesis of Example 49, utilizing propane-2-sulfonyl chloride in place of 2-chloro-3,4-dimethoxybenzoyl chloride in Step 3. ESI-MS m/z 527 (M+H)⁺.

### Example 61: Synthesis of (3R)-3-(2-(cyclopropanesulfonamido)-2-(4-phosphonophenyl)acetamido)-2-hydroxy-3,4-dihydro-2H-benzo[e][1,2]oxaborinine-8-carboxylic acid

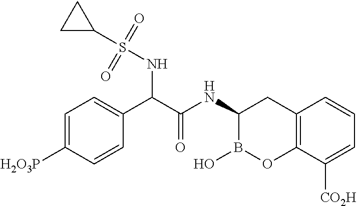

The title compound was prepared in a similar manner to the synthesis of Example 49, utilizing cyclopropanesulfonyl chloride in place of 2-chloro-3,4-dimethoxybenzoyl chloride in Step 3. ESI-MS m/z 525 (M+H)⁺.

### Example 64: Synthesis of (3R)-2-hydroxy-3-(2-((S)-5-oxopyrrolidine-2-carboxamido)-2-(4-phosphonophenyl)acetamido)-3,4-dihydro-2H-benzo[e][1,2]oxaborinine-8-carboxylic acid

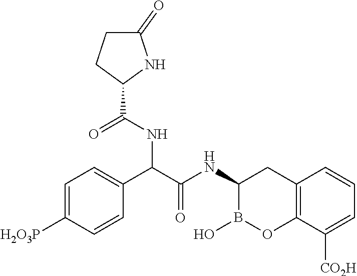

The title compound was prepared in a similar manner to the synthesis of Example 49, utilizing (S)-5-oxopyrrolidine-2-carbonyl chloride in place of 2-chloro-3,4-dimethoxybenzoyl chloride in Step 3. ESI-MS m/z 532 (M+H)⁺.

### Example 65: Synthesis of (3R)-3-(2-((S)-1-acetylpyrrolidine-2-carboxamido)-2-(4-phosphonophenyl)acetamido)-2-hydroxy-3,4-dihydro-2H-benzo[e][1,2]oxaborinine-8-carboxylic acid

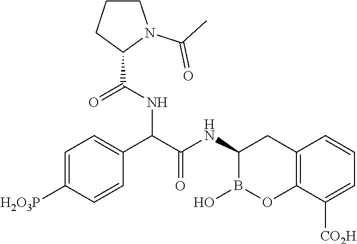

The title compound was prepared in a similar manner to the synthesis of Example 49, utilizing acetyl-L-prolinoyl chloride in place of 2-chloro-3,4-dimethoxybenzoyl chloride in Step 3. ESI-MS m/z 560 (M+H)⁺.

### Example 66: Synthesis of (3R)-2-hydroxy-3-(2-((S)-5-oxopyrrolidine-2-carboxamido)-2-(4-phosphonophenyl)acetamido)-3,4-dihydro-2H-benzo[e][1,2]oxaborinine-8-carboxylic acid

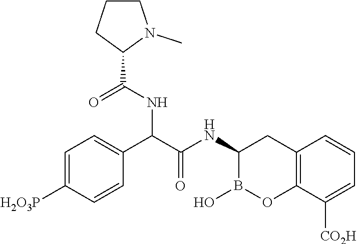

The title compound was prepared in a similar manner to the synthesis of Example 49, utilizing methyl-L-prolinoyl chloride in place of 2-chloro-3,4-dimethoxybenzoyl chloride in Step 3. ESI-MS m/z 532 (M+H)⁺.

### Example 120: Synthesis of (3R)-3-(2-(4-(2-(2-chloro-3,4-dihydroxybenzamido)ethyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 2-(4-(bis(benzyloxy)phosphoryl)phenyl)-2-(4-(2-(2-chloro-3,4-dimethoxybenzamido)ethyl)-2,3-dioxopiperazine-1-carboxamido)acetic acid

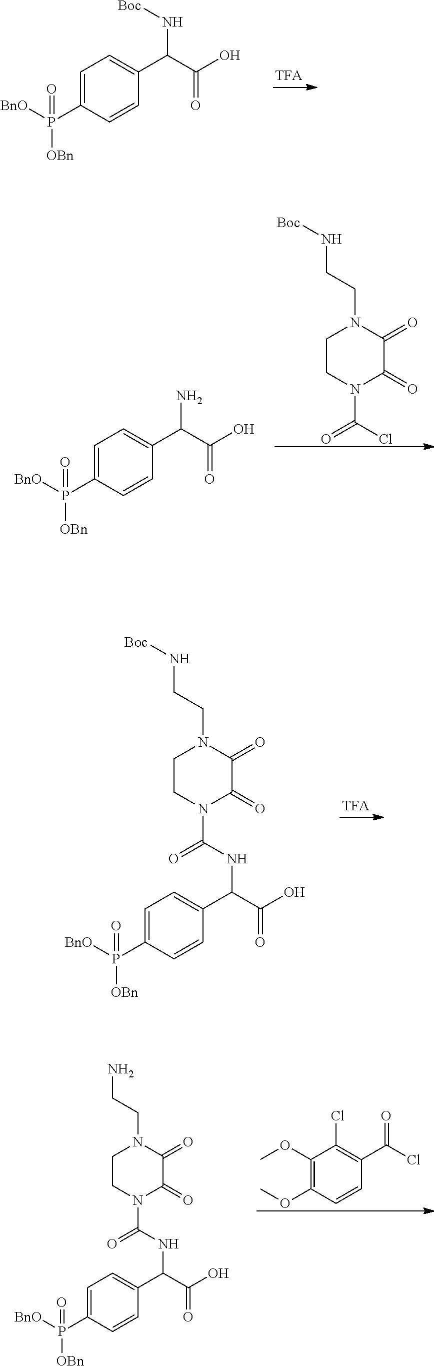 [SMILES: CC(C)(C)OC(=O)NC(C(=O)O)c1ccc(P(=O)(OCc2ccccc2)OCc2ccccc2)cc1 ; NC(C(=O)O)c1ccc(P(=O)(OCc2ccccc2)OCc2ccccc2)cc1 ; CC(C)(C)OC(=O)NCCN1CCN(C(=O)Cl)C(=O)C1=O ; CC(C)(C)OC(=O)NCCN1CCN(C(=O)NC(C(=O)O)c2ccc(P(=O)(OCc3ccccc3)OCc3ccccc3)cc2)C(=O)C1=O ; NCCN1CCN(C(=O)NC(C(=O)O)c2ccc(P(=O)(OCc3ccccc3)OCc3ccccc3)cc2)C(=O)C1=O ; COc1ccc(C(=O)Cl)c(Cl)c1OC]

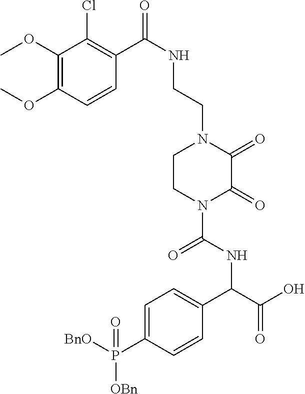 [SMILES: COc1ccc(C(=O)NCCN2CCN(C(=O)NC(C(=O)O)c3ccc(P(=O)(OCc4ccccc4)OCc4ccccc4)cc3)C(=O)C2=O)c(Cl)c1OC]

### Step 1a.

This reaction was performed in a similar manner to Example 8, Step 3a.

### Step 1b.

This reaction was performed in a similar manner to Example 8, Step 3b utilizing tert-butyl (2-(4-(chlorocarbonyl)-2,3-dioxopiperazin-1-yl)ethyl)carbamate in place of 4-(2-chloro-3,4-dimethoxybenzyl)-2,3-dioxopiperazine-1-carbonyl chloride. The product was purified by silica gel chromatography (0-5% MeOH/DCM) to yield desired intermediate (2.73 g, 79% over 2 steps).

### Step 1c.

To a solution of 2-(4-(bis(benzyloxy)phosphoryl)phenyl)-2-(4-(2-((tert-butoxycarbonyl)amino)ethyl)-2,3-dioxopiperazine-1-carboxamido)acetic acid (1.14 g, 1.64 mmol) in DCM (6.6 mL) at 0° C. was added TFA (1.64 mL). The solution was warmed to RT for 1 h then concentrated.

### Step 1d.

The crude product was dissolved in DCM (16 mL). Triethylamine (1.1 mL, 8.2 mmol, 5.0 equiv.) was added followed by 2-chloro-3,4-dimethoxybenzoyl chloride (424 mg, 1.80 mmol, 1.1 equiv.). The mixture was stirred for 1 h then quenched with NaHSO₄ (1.0 M, 10.0 mL). The layers were separated and the aqueous layer was extracted with DCM (3×20 mL). The combined organic layers were dried (Na₂SO₄), filtered, and concentrated. The crude product was purified by silica gel chromatography (0-10% MeOH/DCM) to yield title compound (1.01 g, 78% over 2 steps).

### Step 2. Synthesis of (3R)-3-(2-(4-(2-(2-chloro-3,4-dihydroxybenzamido)ethyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 8, Step 4 utilizing (3R)-3-(2-(4-(2-(2-chloro-3,4-dihydroxybenzamido)ethyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid in place of 2-(4-(bis(benzyloxy)phosphoryl)phenyl)-2-(4-(2-chloro-3,4-dimethoxybenzyl)-2,3-dioxopiperazine-1-carboxamido)acetic acid. The desired compound was isolated by reverse-phase HPLC. ESI-MS m/z 791 (M+H)⁺.

### Example 160: Synthesis of (R)-3-((R)-2-(4-(2-chloro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 2-(4-(bis(benzyloxy)phosphoryl)phenyl)-2-((tert-butoxycarbonyl)amino)acetic acid

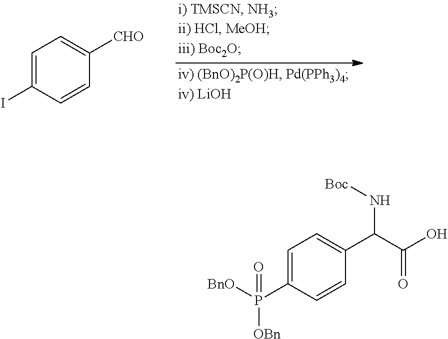 [SMILES: O=Cc1ccc(I)cc1 ; CC(C)(C)OC(=O)NC(C(=O)O)c1ccc(P(=O)(OCc2ccccc2)OCc2ccccc2)cc1]

### Step 1a.

To 4-iodobenzaldehyde (46.2 g, 200 mmol) at 0° C. was added 7 N ammonia in methanol (600 mL), followed by trimethylsilyl cyanide (29.8 g, 300 mmol, 1.5 eq). The mixture was stirred at 45° C. for 24 h and concentrated in vacuo.

### Step 1b.

The crude product was dissolved in 3 N hydrochloric acid in methanol (400 mL), stirred at 50° C. for 36 h and concentrated in vacuo.

### Step 1c.

To the crude product in DCM (400 mL) at 0° C. was added triethylamine (30.3 g, 300 mmol, 3 eq) followed by di-tert-butyl dicarbonate (65.4 g, 300 mmol, 1.5 eq). The reaction was warmed to RT for 12 h and concentrated in vacuo. The crude product was purified by flash silica gel chromatography (10% ethyl acetate/hexanes) to give the desired intermediate (51 g, 66% for 3 steps).

### Step 1d.

To methyl 2-((tert-butoxycarbonyl)amino)-2-(4-iodophenyl)acetate (20 g, 52 mmol) was added N,N-diisopropylethylamine 20.2 g, 156 mmol, 3 eq), Pd(PPh₃)₄ (11.8 g, 10.2 mmol, 20 mol %), dibenzyl phosphite (26.7 g, 102 mmol, 2 eq), followed by anhydrous toluene (400 mL) and the mixture was stirred at RT for 5-7 h under argon. The reaction was diluted with ethyl acetate, washed with water then brine, dried over sodium sulfate, and concentrated. The product was purified by flash chromatography on silica gel (35-50% ethyl acetate/hexanes) to give the desired intermediate (27 g, 99%).

### Step 1e.

To methyl 2-(4-(bis(benzyloxy)phosphoryl)phenyl)-2-((tert-butoxycarbonyl)amino)acetate (27 g, 51.4 mmol) in THF (100 mL)/H₂O (100 mL) was added lithium hydroxide monohydrate (6.48 g, 154 mmol, 3 eq) and the mixture was stirred at RT for 1 h. 2 N Hydrochloric acid was added drop wise to obtain pH 2 and extracted with dichloromethane. The organic layer was washed with water, dried over sodium sulfate, and concentrated to give 20 g crude product. The product was purified by flash chromatography on silica gel (10-20% MeOH/DCM) to give the title compound (15.8 g, 60%) as a white solid. ESI-MS m/z 512 (M+H)⁺.

### Step 2. Synthesis of 4-(2-chloro-3,4-dimethoxybenzyl)-2,3-dioxopiperazine-1-carbonyl chloride

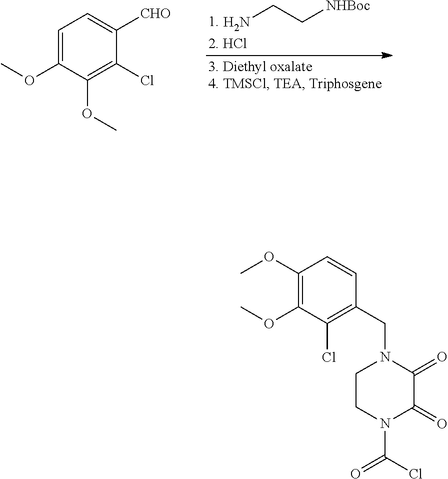 [SMILES: COc1ccc(C=O)c(Cl)c1OC ; CC(C)(C)OC(=O)NCCN ; COc1ccc(CN2CCN(C(=O)Cl)C(=O)C2=O)c(Cl)c1OC]

### Step 2a.

To a solution of 2-chloro-3,4-dimethoxybenzaldehyde (3.0 g, 15 mmol) in MeOH (500 mL) was added tert-butyl (2-aminoethyl)carbamate (2.6 mL, 16 mmol, 1.1 equiv.). The solution was stirred at RT for 2 h then cooled to 0° C. NaBH₄ (2.8 g, 75 mmol, 5.0 equiv.) was added portion wise and allowed to warm to RT overnight. The mixture was concentrated, followed by H₂O (200 mL) and DCM (200 mL) addition. The layers were separated, and the aq. layer was extracted with DCM (3×100 mL). The combined organic layers were dried (Na₂SO₄), filtered, and concentrated.

### Step 2b.

To the crude product in MeOH (30 mL) at 0° C. was added HCl (4 M in Dioxane, 15 mL). The reaction was warmed to RT and stirred for 2 h before being concentrated in vacuo.

### Step 2c.

The crude product was dissolved in EtOH (150 mL) and TEA (6.3 mL, 45 mmol, 3.0 equiv.), and diethyl oxalate (2.2 mL, 16 mmol, 1.1 equiv.) were added sequentially. The mixture was heated to 90° C. overnight and concentrated. The crude product was purified by silica gel chromatography (0-5% MeOH/DCM) to yield desired intermediate (2.0 g, 45% over 3 steps).

### Step 2d.

A solution of 1-(2-chloro-3,4-dimethoxybenzyl)piperazine-2,3-dione (2.0 g, 6.7 mmol) in DCM (10 mL)/THF (10 mL) was cooled to −40° C. TMSCl (0.93 mL, 7.4 mmol, 1.1 equiv.) and TEA (1.1 mL, 8.0 mmol, 1.2 equiv.) were added sequentially. The mixture was warmed to 0° C. for 1 h. Triphosgene (795 mg, 2.7 mmol, 0.4 equiv.) in THF (10 mL) was added and warmed to RT for 1 h. The heterogeneous mixture was filtered through Celite and concentrated in vacuo to provide a sticky brown oil. Et₂O (50 mL) was added and the product was triturated at RT overnight and filtered. The resulting solid was washed with Et₂O (10 mL) and dried in vacuo to provide the title compound (2.0 g, 84%).

### Step 3. Synthesis of 2-(4-(bis(benzyloxy)phosphoryl)phenyl)-2-(4-(2-chloro-3,4-dimethoxybenzyl)-2,3-dioxopiperazine-1-carboxamido)acetic acid

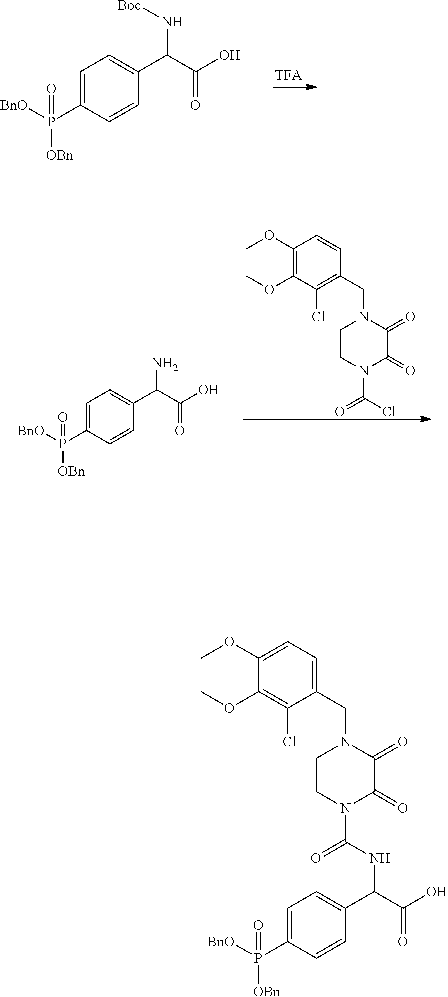 [SMILES: CC(C)(C)OC(=O)NC(C(=O)O)c1ccc(P(=O)(OCc2ccccc2)OCc2ccccc2)cc1 ; NC(C(=O)O)c1ccc(P(=O)(OCc2ccccc2)OCc2ccccc2)cc1 ; COc1ccc(CN2CCN(C(=O)Cl)C(=O)C2=O)c(Cl)c1OC ; COc1ccc(CN2CCN(C(=O)NC(C(=O)O)c3ccc(P(=O)(OCc4ccccc4)OCc4ccccc4)cc3)C(=O)C2=O)c(Cl)c1OC]

### Step 3a.

To a solution of 2-(4-(bis(benzyloxy)phosphoryl)phenyl)-2-((tert-butoxycarbonyl)amino)acetic acid (500 mg, 0.98 mmol) in DCM (4.0 mL) at 0° C. was added TFA (1.0 mL). The solution was warmed to RT for 2 h then concentrated.

### Step 3b.

The crude product was dissolved in THF (4.0 mL)/NaHCO₃ (sat. aq., 4.0 mL) followed by addition of 4-(2-chloro-3,4-dimethoxybenzyl)-2,3-dioxopiperazine-1-carbonyl chloride (388 mg, 1.07 mmol, 1.1 equiv.). The reaction was stirred at RT for 2 h. The reaction was diluted with H₂O (5 mL) and acidified until pH 2 with HCl (2 M). Ethyl acetate (50 mL) was added and the organic layer was washed with H₂O (2×10 mL) and brine (1×10 mL), dried (Na₂SO₄), and concentrated. The crude product was purified by silica gel chromatography (0-20% MeOH/DCM) to yield title compound (375 mg, 69% over 2 steps). ESI-MS m/z 737 (M+H)⁺.

### Step 4. Synthesis of (R)-3-((R)-2-(4-(2-chloro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

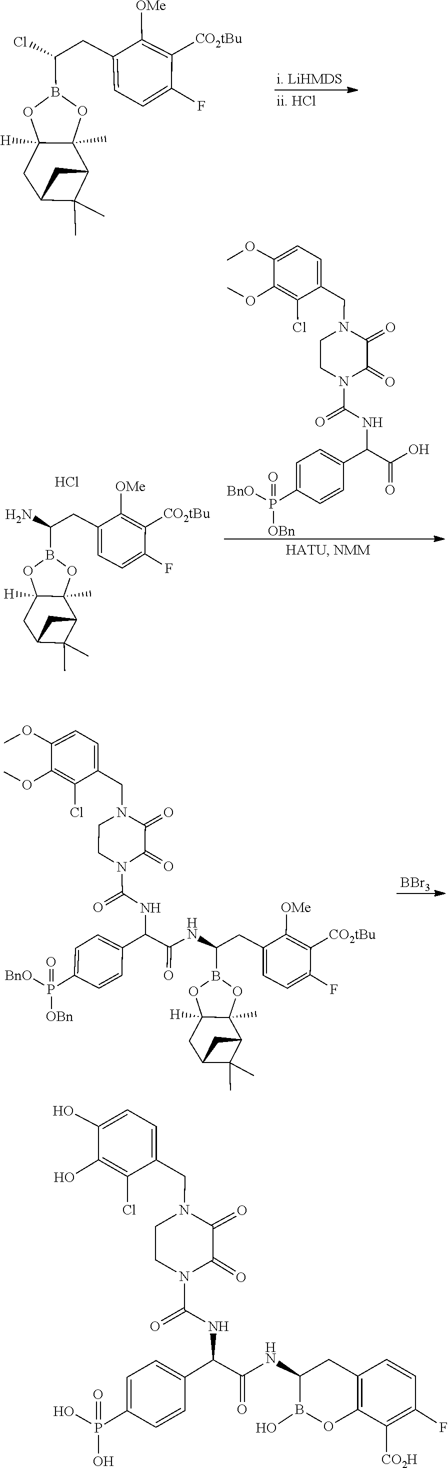

### Step 4a.

A solution of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (5.2 g, 11 mmol) (Example 200, Step 7) in THF (45 mL) was cooled to −78° C. LiHMDS (1 M in THF, 11 mL, 11 mmol, 1.0 equiv.) was added dropwise and the mixture was warmed to RT for 1 h. The solution was cooled to 0° C. and HCl (4 M in dioxane, 11 mL, 44 mmol, 4.0 equiv.) was added dropwise. The reaction was warmed to RT for 1 h then concentrated. Hexanes (200 mL) was added and stirred at RT overnight. The solid was filtered, washed with hexanes (2×100 mL), and dried to yield desired intermediate tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (5.8 g, 99%).

### Step 4b.

To a solution of tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (198 mg, 0.340 mmol), 2-(4-(bis(benzyloxy)phosphoryl)phenyl)-2-(4-(2-chloro-3,4-dimethoxybenzyl)-2,3-dioxopiperazine-1-carboxamido)acetic acid (375 mg, 0.509 mmol, 1.5 equiv.), and HATU (226 mg, 0.595 mmol, 1.75 equiv.) in DMA (3.4 mL) was added NMM (0.11 mL, 1.02 mmol, 3.0 equiv.). The reaction was stirred at RT for 30 min. and diluted with EtOAc (30 mL). The mixture was washed with H₂O (2×10 mL) and brine (xx mL), dried (Na₂SO₄), filtered, and concentrated. The crude product was purified by silica gel chromatography (0-5% MeOH/DCM) to yield desired tert-butyl 3-((2R)-2-(2-(4-(bis(benzyloxy)phosphoryl)phenyl)-2-(4-(2-chloro-3,4-dimethoxybenzyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (252 mg, 64%) ESI m z 1166 (M+H)⁺.

### Step 4c.

To a solution of tert-butyl 3-((2R)-2-(2-(4-(bis(benzyloxy)phosphoryl)phenyl)-2-(4-(2-chloro-3,4-dimethoxybenzyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (252 mg, 0.216 mmol) in DCM (2.2 mL) at −78° C. was added BBr₃ (1 M in DCM, 2.2 mL, 2.2 mmol, 10 equiv.). The reaction was warmed to RT overnight then concentrated. The resulting crude product was purified by reverse-phase HPLC to yield (3R)-3-(2-(4-(2-chloro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid (44 mg, 28%). ESI m/z 735 (M+H)⁺. After reversed phase HPLC purification, the title compound was collected as the second eluting peak ESI m/z 735 (M+H)⁺.

### Example 161: Synthesis of (R)-3-((S)-2-(4-(2-chloro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 160, utilizing tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in Step 2. After reversed phase HPLC purification in Step 5, the title compound was collected as the first eluting peak. ESI m/z 735 (M+H)⁺.

### Example 162: Synthesis of (R)-3-((S)-2-(4-(2-chloro-3,4-dihydroxybenzyl)-2,3-dioxopiperazine-1-carboxamido)-2-(3-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 8, utilizing 3-iodobenzaldehyde in place of 4-iodobenzaldehyde in Step 1a. The title compound was isolated as the second eluting peak by reverse-phase HPLC. ESI-MS m/z 734 (M+H)⁺.

### Example 163: Synthesis of (R)-3-((R)-2-(4-(2-chloro-3,4-dihydroxybenzyl)-2,3-dioxopiperazine-1-carboxamido)-2-(3-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 8, utilizing 3-iodobenzaldehyde in place of 4-iodobenzaldehyde in Step Ia. The title compound was isolated as the first eluting peak by reverse-phase HPLC. ESI-MS m/z 734 (M+H)⁺.

### Example 164: Synthesis of (3R)-3-(2-(4-(2-chloro-5-fluoro-3,4-dihydroxybenzyl)-2,3-dioxopiperazine-1-carboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 45, tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. The title compound was isolated by reverse-phase HPLC. ESI-MS m/z 734 (M+H)⁺.

### Example 165: Synthesis of (R)-3-((S)-2-(4-(2-chloro-3,4-dihydroxybenzyl)-2,3-dioxopiperazine-1-carboxamido)-2-(2,3-difluoro-4-phosphonophenyl)acetamido)-7-fluoro-2-hydroxy-3,4-dihydro-2H-benzo[e][1,2]oxaborinine-8-carboxylic acid, and

### Example 166: Synthesis of (R)-3-((R)-2-(4-(2-chloro-3,4-dihydroxybenzyl)-2,3-dioxopiperazine-1-carboxamido)-2-(2,3-difluoro-4-phosphonophenyl)acetamido)-7-fluoro-2-hydroxy-3,4-dihydro-2H-benzo[e][1,2]oxaborinine-8-carboxylic acid

The title compounds were prepared in a similar manner to the synthesis of Example 8, utilizing 2,3-difluoro-4-iodobenzaldehyde in place of 4-iodobenzaldehyde in Step Ia. The title compounds were isolated by reverse-phase HPLC. ESI-MS m/z 770 (M+H)⁺.

### Example 167: Synthesis of (R)-3-((S)-2-(3-((2-(2-chloro-3,4-dihydroxybenzamido)ethyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid, and

### Example 168: Synthesis of (R)-3-((R)-2-(3-((2-(2-chloro-3,4-dihydroxybenzamido)ethyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 2-(4-(bis(benzyloxy)phosphoryl)phenyl)-2-((tert-butoxycarbonyl)amino)acetic acid

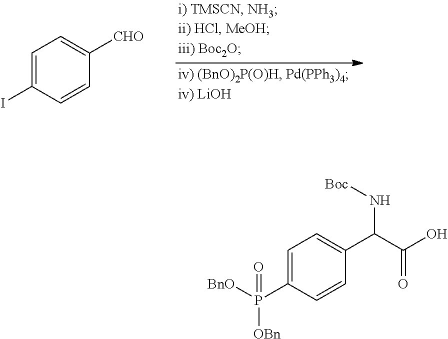 [SMILES: O=Cc1ccc(I)cc1 ; CC(C)(C)OC(=O)NC(C(=O)O)c1ccc(P(=O)(OCc2ccccc2)OCc2ccccc2)cc1]

### Step 1a.

To 4-iodobenzaldehyde (46.2 g, 200 mmol) at 0° C. was added 7 N ammonia in methanol (600 mL), followed by trimethylsilyl cyanide (29.8 g, 300 mmol, 1.5 eq). The mixture was stirred at 45° C. for 24 h and concentrated in vacuo.

### Step 1b.

The crude product was dissolved in 3 N hydrochloric acid in methanol (400 mL), stirred at 50° C. for 36 h and concentrated in vacuo.

### Step 1c.

To the crude product in DCM (400 mL) at 0° C. was added triethylamine (30.3 g, 300 mmol, 3 eq) followed by di-tert-butyl dicarbonate (65.4 g, 300 mmol, 1.5 eq). The reaction was warmed to RT for 12 h and concentrated in vacuo. The crude product was purified by flash silica gel chromatography (10% ethyl acetate/hexanes) to give the desired intermediate (51 g, 66% for 3 steps).

### Step 1d.

To methyl 2-((tert-butoxycarbonyl)amino)-2-(4-iodophenyl)acetate (20 g, 52 mmol) was added N,N-diisopropylethylamine 20.2 g, 156 mmol, 3 eq), Pd(PPh₃)₄ (11.8 g, 10.2 mmol, 20 mol %), dibenzyl phosphite (26.7 g, 102 mmol, 2 eq), followed by anhydrous toluene (400 mL) and the mixture was stirred at RT for 5-7 h under argon. The reaction was diluted with ethyl acetate, washed with water then brine, dried over sodium sulfate, and concentrated. The product was purified by flash chromatography on silica gel (35-50% ethyl acetate/hexanes) to give the desired intermediate (27 g, 99%).

### Step 1e.

To methyl 2-(4-(bis(benzyloxy)phosphoryl)phenyl)-2-((tert-butoxycarbonyl)amino)acetate (27 g, 51.4 mmol) in THF (100 mL)/H₂O (100 mL) was added lithium hydroxide monohydrate (6.48 g, 154 mmol, 3 eq) and the mixture was stirred at RT for 1 h. 2 N Hydrochloric acid was added drop wise to obtain pH 2 and extracted with dichloromethane. The organic layer was washed with water, dried over sodium sulfate, and concentrated to give 20 g crude product. The product was purified by flash chromatography on silica gel (10-20% MeOH/DCM) to give the title compound (15.8 g, 60%) as a white solid. ESI-MS m/z 512 (M+H)⁺.

### Step 2. Synthesis of tert-butyl (2-((3-chloro-2-oxoimidazolidin-1-yl)sulfonyl)ethyl)carbamate

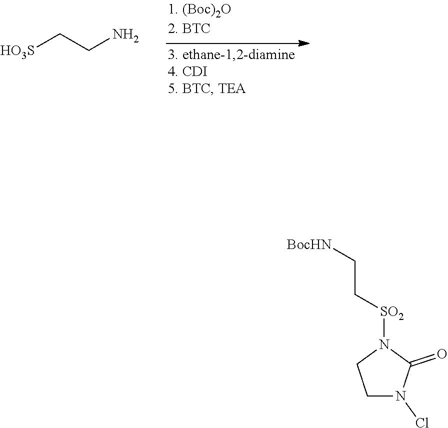 [SMILES: NCCS(=O)(=O)O ; CC(C)(C)OC(=O)NCCS(=O)(=O)N1CCN(Cl)C1=O]

### Step 2a.

To a solution of 2-aminoethane-1-sulfonic in tetrabutylammonium hydroxide/acetone/water was added (Boc)₂O. The solution was stirred at RT overnight. The mixture was concentrated, followed by DCM addition. The layers were separated and the aq. layer was extracted with DCM. The combined organic layers were dried (Na₂SO₄), filtered, and concentrated to give crude product.

### Step 2b.

To the crude product in THF at 0° C. was added BTC, then the mixture was stirred at room temperature for 30 min. The mixture was concentrated under reduced pressure and the residue was dissolved in EtOAc/hexane (1:1 v/v) and the mixture was filtered through a small amount of silica gel with EtOAc/hexane (1:1, v/v) as an eluent. Evaporate the mixture under reduced pressure to give tert-butyl (2-(chlorosulfonyl)ethyl)carbamate.

### Step 2c.

The tert-butyl (2-(chlorosulfonyl)ethyl)carbamate was dissolved in ethane-1,2-diamine was stirred at RT overnight. The combined organic layers were dried (Na₂SO₄), filtered, and concentrated to give crude tert-butyl (2-(N-(2-aminoethyl)sulfamoyl)ethyl)carbamate.

### Step 2d.

A solution tert-butyl (2-(N-(2-aminoethyl)sulfamoyl)ethyl)carbamate in THF was cooled to 0° C. CDI was added. The mixture was warmed to 60° C. for 4 h. The heterogeneous mixture was filtered through Celite and concentrated in vacuo to provide light yellow oil. Purified by flash chromatography to provide tert-butyl (2-((2-oxoimidazolidin-1-yl)sulfonyl)ethyl)carbamate.

### Step 2e.

A solution of tert-butyl (3-((2-oxoimidazolidin-1-yl)sulfonyl)propyl) and TEA in THF was cooled to 0° C. Triphosgene in THF was added and warmed to RT for 1 h. The heterogeneous mixture was filtered through Celite and concentrated in vacuo to provide the title compound.

### Step 3. Synthesis of 2-(4-(bis(benzyloxy)phosphoryl)phenyl)-2-(3-((2-((tert-butoxycarbonyl)amino)ethyl)sulfonyl)-2-oxoimidazolidine-1-carboxamido)acetic acid

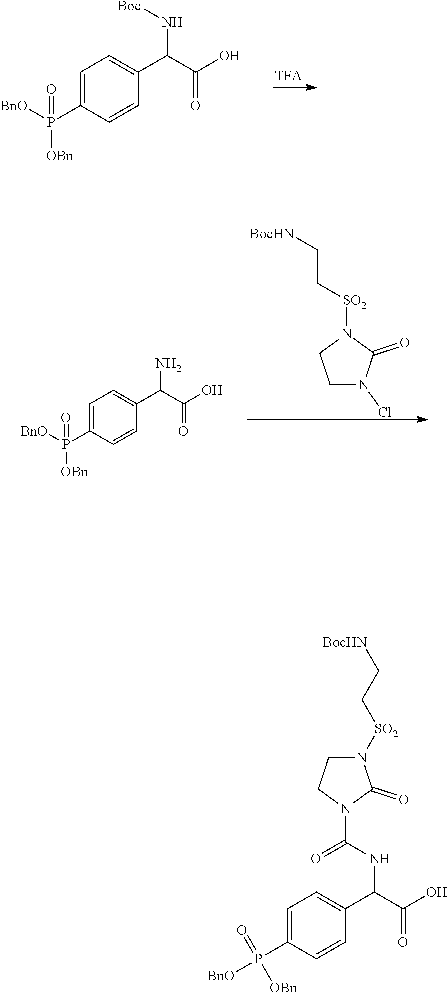 [SMILES: CC(C)(C)OC(=O)NC(C(=O)O)c1ccc(P(=O)(OCc2ccccc2)OCc2ccccc2)cc1 ; NC(C(=O)O)c1ccc(P(=O)(OCc2ccccc2)OCc2ccccc2)cc1 ; CC(C)(C)OC(=O)NCCS(=O)(=O)N1CCN(Cl)C1=O ; CC(C)(C)OC(=O)NCCS(=O)(=O)N1CCN(C(=O)NC(C(=O)O)c2ccc(P(=O)(OCc3ccccc3)OCc3ccccc3)cc2)C1=O]

### Step 3a.

To a solution of 2-(4-(bis(benzyloxy)phosphoryl)phenyl)-2-((tert-butoxycarbonyl)amino)acetic acid (5 g, 0.98 mmol) in DCM (40 mL) at 0° C. was added TFA (10 mL). The solution was warmed to RT for 2 h then concentrated.

### Step 3b.

The crude product was dissolved in THF (40 mL)/NaHCO₃ (sat. aq., 40 mL) followed by addition of tert-butyl (2-((3-chloro-2-oxoimidazolidin-1-yl)sulfonyl)ethyl)carbamate (3.8 g, 10.7 mmol, 1.1 equiv.). The reaction was stirred at RT for 2 h. The reaction was diluted with H₂O (50 mL) and acidified until pH 2 with HCl (2 M). Ethyl acetate (500 mL) was added and the organic layer was washed with H₂O (2×100 mL) and brine (1×100 mL), dried (Na₂SO₄), and concentrated. The crude product was purified by silica gel chromatography (0-20% MeOH/DCM) to yield title compound (5.01 g, 70% over 2 steps). ESI-MS m/z 731 (M+H)⁺.

### Step 4. Synthesis of 2-(4-(bis(benzyloxy)phosphoryl)phenyl)-2-(3-((2-(2-chloro-3,4-dimethoxybenzamido)ethyl)sulfonyl)-2-oxoimidazolidine-1-carboxamido)acetic acid

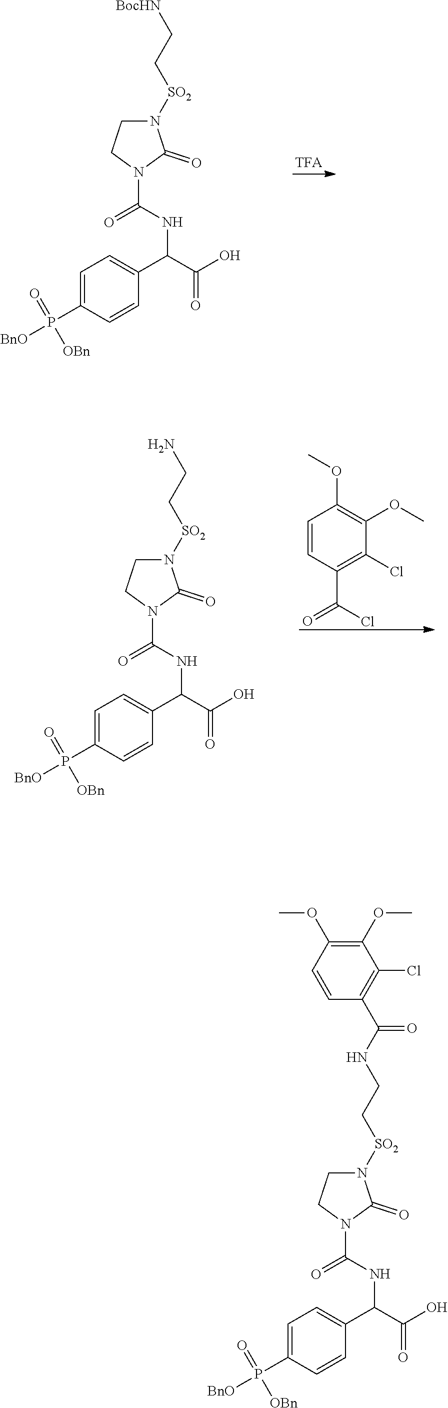 [SMILES: CC(C)(C)OC(=O)NCCS(=O)(=O)N1CCN(C(=O)NC(C(=O)O)c2ccc(P(=O)(OCc3ccccc3)OCc3ccccc3)cc2)C1=O ; NCCS(=O)(=O)N1CCN(C(=O)NC(C(=O)O)c2ccc(P(=O)(OCc3ccccc3)OCc3ccccc3)cc2)C1=O ; COc1ccc(C(=O)Cl)c(Cl)c1OC ; COc1ccc(C(=O)NCCS(=O)(=O)N2CCN(C(=O)NC(C(=O)O)c3ccc(P(=O)(OCc4ccccc4)OCc4ccccc4)cc3)C2=O)c(Cl)c1OC]

### Step 4a.

To a solution of 2-(4-(bis(benzyloxy)phosphoryl)phenyl)-2-(3-((2-((tert-butoxycarbonyl)amino)ethyl)sulfonyl)-2-oxoimidazolidine-1-carboxamido)acetic acid (730 mg, 1 mmol) in DCM (4.0 mL) at 0° C. was added TFA (1.0 mL). The solution was warmed to RT for 2 h then concentrated.

### Step 4b.

The crude product was dissolved in THF (4.0 mL)/NaHCO₃ (sat. aq., 4.0 mL) followed by addition of 2-chloro-3,4-dimethoxybenzoyl chloride (258 mg, 1.1 mmol, 1.1 equiv.). The reaction was stirred at RT for 2 h. The reaction was diluted with H₂O (5 mL) and acidified until pH 2 with HCl (2 M). Ethyl acetate (50 mL) was added and the organic layer was washed with H₂O (2×10 mL) and brine (1×10 mL), dried (Na₂SO₄), and concentrated. The crude product was purified by silica gel chromatography (0-20% MeOH/DCM) to yield title compound (580 mg, 70% over 2 steps). ESI-MS m/z 829 (M+H)⁺.

### Step 5. Synthesis of (3R)-3-(2-(3-((2-(2-chloro-3,4-dihydroxybenzamido)ethyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

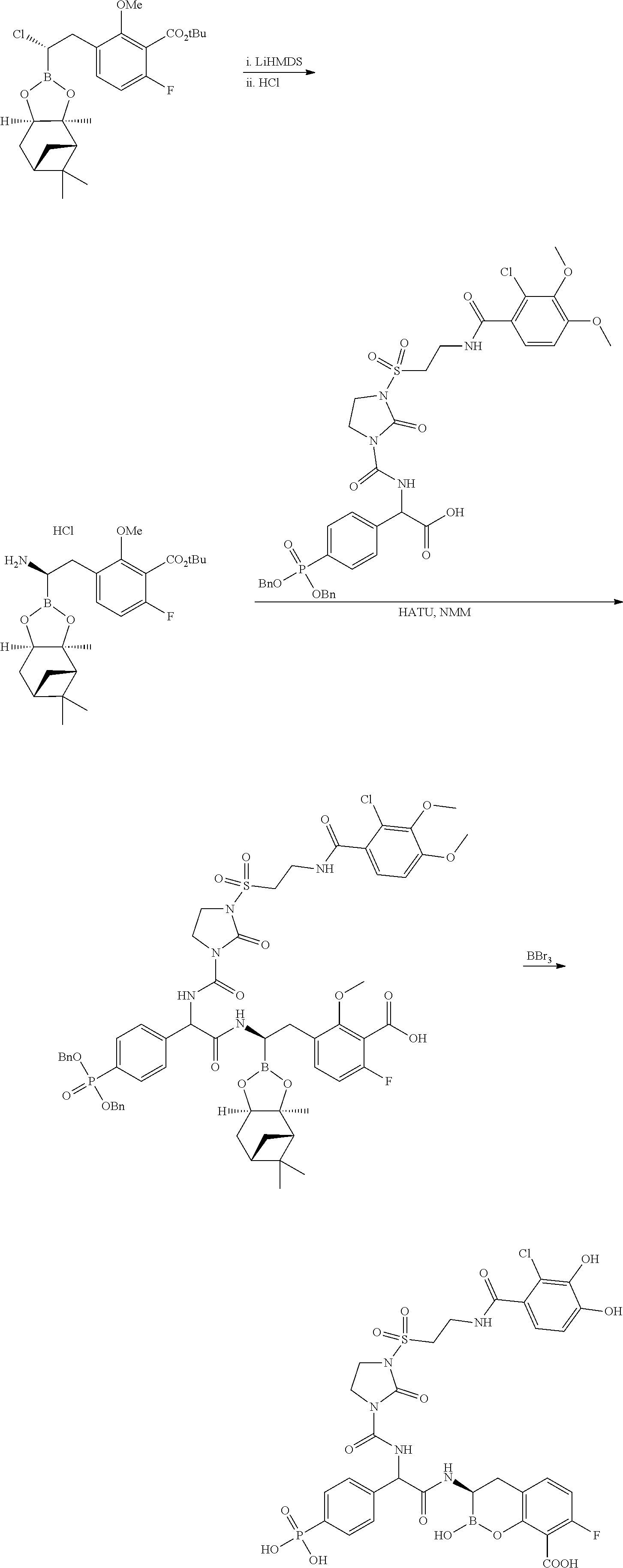

### Step 5a.

A solution of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (5.2 g, 11 mmol) in THF (45 mL) was cooled to −78° C. LiHMDS (1 M in THF, 11 mL, 11 mmol, 1.0 equiv.) was added dropwise and the mixture was warmed to RT for 1 h. The solution was cooled to 0° C. and HCl (4 M in dioxane, 11 mL, 44 mmol, 4.0 equiv.) was added dropwise. The reaction was warmed to RT for 1 h then concentrated. Hexanes (200 mL) was added and stirred at RT overnight. The solid was filtered, washed with hexanes (2×100 mL), and dried to yield desired intermediate tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (5.8 g, 99%).

### Step 5b.

To a solution of tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (198 mg, 0.340 mmol), 2-(4-(bis(benzyloxy)phosphoryl)phenyl)-2-(3-((2-(2-chloro-3,4-dimethoxybenzamido)ethyl)sulfonyl)-2-oxoimidazolidine-1-carboxamido)acetic acid (422 mg, 0.509 mmol, 1.5 equiv.), and HATU (226 mg, 0.595 mmol, 1.75 equiv.) in DMA (3.4 mL) was added NMM (0.11 mL, 1.02 mmol, 3.0 equiv.). The reaction was stirred at RT for 30 min. and diluted with EtOAc (30 mL). The mixture was washed with H₂O (2×10 mL) and brine (xx mL), dried (Na₂SO₄), filtered, and concentrated. The crude product was purified by silica gel chromatography (0-5% MeOH/DCM) to yield desired 3-((2R)-2-(2-(4-(bis(benzyloxy)phosphoryl)phenyl)-2-(3-((2-(2-chloro-3,4-dimethoxybenzamido)ethyl)sulfonyl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoic acid (260 mg, 64%) ESI m/z 1202 (M+H)⁺.

### Step 5c.

To a solution of 3-((2R)-2-(2-(4-(bis(benzyloxy)phosphoryl)phenyl)-2-(3-((2-(2-chloro-3,4-dimethoxybenzamido)ethyl)sulfonyl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoic acid (260 mg, 0.216 mmol) in DCM (2.2 mL) at −78° C. was added BBr₃ (1 M in DCM, 2.2 mL, 2.2 mmol, 10 equiv.). The reaction was warmed to RT overnight then concentrated. The resulting crude product was purified by reverse-phase HPLC to yield (3R)-3-(2-(3-((2-(2-chloro-3,4-dihydroxybenzamido)ethyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid (50 mg, 28%). ESI m/z 828 (M+H)⁺. After reversed phase HPLC purification, the title compound was collected as the second eluting peak ESI m/z 828 (M+H)⁺.

### Example 169: Synthesis of (R)-3-((S)-2-(3-((3-(2-chloro-3,4-dihydroxybenzamido)propyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

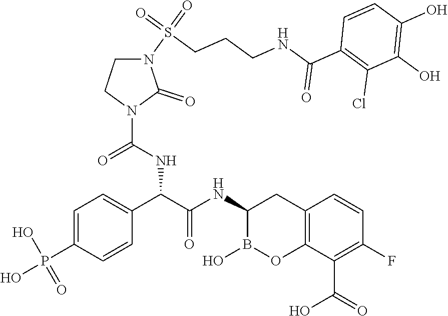 [SMILES: O=C(NCCCS(=O)(=O)N1CCN(C(=O)N[C@H](C(=O)N[C@H]2Cc3ccc(F)c(C(=O)O)c3OB2O)c2ccc(P(=O)(O)O)cc2)C1=O)c1ccc(O)c(O)c1Cl]

The title compound was prepared in a similar manner to the synthesis of Example 168, utilizing 3-aminopropane-1-sulfonic acid in place of 2-aminoethane-1-sulfonic in Step 2. After reversed phase HPLC purification, the title compound was collected as the first eluting peak ESI m z 842 (M+H)⁺.

### Example 170: Synthesis of (R)-3-((R)-2-(3-((2-(2-chloro-3,4-dihydroxybenzamido)ethyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

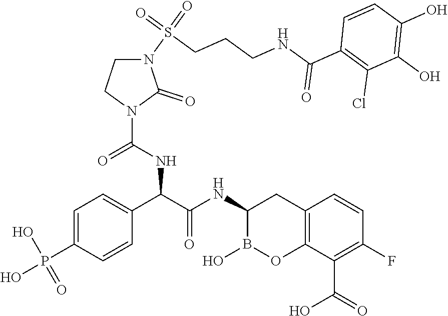 [SMILES: O=C(NCCCS(=O)(=O)N1CCN(C(=O)N[C@@H](C(=O)N[C@H]2Cc3ccc(F)c(C(=O)O)c3OB2O)c2ccc(P(=O)(O)O)cc2)C1=O)c1ccc(O)c(O)c1Cl]

The title compound was prepared in a similar manner to the synthesis of Example 168 and collected as the first eluting peak after reversed phase HPLC purification. ESI-MS m/z 828 (M+H)⁺.

### Example 171: Synthesis of (R)-3-((R)-2-amino-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 1, utilizing 4-iodobenzaldehyde in place of 2-fluoro-4-iodobenzaldehyde in Step 1. After reversed phase HPLC purification, the title compound was collected as the first eluting peak. ESI-MS m/z 421 (M+H)⁺.

### Example 172: Synthesis of (3R)-2-hydroxy-3-(2-(4-phosphonophenyl)-2-(2,2,2-trifluoroacetamido)acetamido)-3,4-dihydro-2H-benzo[e][1,2]oxaborinine-8-carboxylic acid

The title compound was prepared in a similar manner to the synthesis of Example 49, utilizing 2,2,2-trifluoroacetyl chloride in place of 2-chloro-3,4-dimethoxybenzoyl chloride in Step 3. ESI-MS m/z 517 (M+H)⁺.

### Example 173: Synthesis of (R)-2-hydroxy-3-((R)-2-(4-phosphonophenyl)-2-((S)-pyrrolidine-2-carboxamido)acetamido)-3,4-dihydro-2H-benzo[e][1,2]oxaborinine-8-carboxylic acid

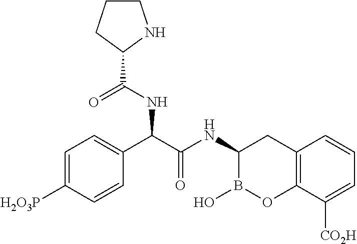

The title compound was prepared in a similar manner to the synthesis of Example 49, utilizing tert-butyl (S)-2-(chlorocarbonyl)pyrrolidine-1-carboxylate in place of 2-chloro-3,4-dimethoxybenzoyl chloride in Step 3. ESI-MS m/z 518 (M+H)⁺.

### Example 174: Synthesis of (3R)-2-hydroxy-3-(2-(2-oxo-1,2-dihydropyridine-3-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing 2-oxo-1,2-dihydropyridine-3-carbonyl chloride in place of 2-chloro-3,4-dimethoxybenzoyl chloride in Step 3. ESI-MS m/z 542 (M+H)⁺.

### Example 175: Synthesis of (R)-2-hydroxy-3-((R)-2-(4-oxo-1,4-dihydropyridine-3-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing 4-oxo-1,4-dihydropyridine-3-carbonyl chloride in place of 2-chloro-3,4-dimethoxybenzoyl chloride in Step 3. ESI-MS m/z 542 (M+H)⁺.

### Example 176: Synthesis of (3R)-2-hydroxy-3-(2-(6-oxo-1,6-dihydropyrimidine-5-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing 6-oxo-1,6-dihydropyrimidine-5-carbonyl chloride in place of 2-chloro-3,4-dimethoxybenzoyl chloride in Step 3. ESI-MS m/z 543 (M+H)⁺.

### Example 177: Synthesis of (R)-2-hydroxy-3-((S)-2-(3-oxo-2,3-dihydropyridazine-4-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing 3-oxo-2,3-dihydropyridazine-4-carbonyl chloride in place of 2-chloro-3,4-dimethoxybenzoyl chloride in Step 3. After reversed phase HPLC purification, the title compound was collected as the first eluting peak ESI m/z 543 (M+H)⁺.

### Example 178: Synthesis of (R)-2-hydroxy-3-((R)-2-(3-oxo-2,3-dihydropyridazine-4-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 178 and collected as the second eluting peak after reversed phase HPLC purification. ESI-MS m/z 543 (M+H)⁺.

### Example 179: Synthesis of (3R)-3-(2-(4-cyanopicolinamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 49, utilizing 4-cyanopicolinoyl chloride in place of 2-chloro-3,4-dimethoxybenzoyl chloride in Step 3. ESI-MS m/z 551 (M+H)⁺.

### Example 180: Synthesis of (R)-3-((R)-2-(6-cyanopicolinamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 49, utilizing 6-cyanopicolinoyl chloride in place of 2-chloro-3,4-dimethoxybenzoyl chloride in Step 3. ESI-MS m/z 551 (M+H)⁺.

### Example 181: Synthesis of (R)-2-hydroxy-3-((S)-2-(1-methyl-2-oxo-1,2-dihydropyridine-3-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing 1-methyl-2-oxo-1,2-dihydropyridine-3-carbonyl chloride in place of 2-chloro-3,4-dimethoxybenzoyl chloride in Step 3. After reversed phase HPLC purification, the title compound was collected as the first eluting peak ESI m/z 556 (M+H)⁺.

### Example 182: Synthesis of (R)-2-hydroxy-3-((R)-2-(1-methyl-2-oxo-1,2-dihydropyridine-3-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 181 and collected as the second eluting peak after reversed phase HPLC purification. ESI-MS m/z 556 (M+H)⁺.

### Example 183 Synthesis of (3R)-3-(2-(3-chloropicolinamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 49, utilizing 3-chloropicolinoyl chloride in place of 2-chloro-3,4-dimethoxybenzoyl chloride in Step 3. ESI-MS 560 m/z (M+H)⁺.

### Example 184: Synthesis of (3R)-3-(2-(5-chloropicolinamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 49, utilizing 5-chloropicolinoyl chloride in place of 2-chloro-3,4-dimethoxybenzoyl chloride in Step 3. ESI-MS m/z 560 (M+H)⁺.

### Example 186: Synthesis of (R)-2-hydroxy-3-((S)-2-(4-phosphonophenyl)-2-(pyrazolo[1,5-a]pyrimidine-6-carboxamido)acetamido)-3,4-dihydro-2H-benzo[e][1,2]oxaborinine-8-carboxylic acid

The title compound was prepared in a similar manner to the synthesis of Example 49, utilizing pyrazolo[1,5-a]pyrimidine-6-carbonyl chloride in place of 2-chloro-3,4-dimethoxybenzoyl chloride in Step 3. After reversed phase HPLC purification, the title compound was collected as the first eluting peak ESI m/z 566 (M+H)⁺.

### Example 187: Synthesis of (R)-2-hydroxy-3-((R)-2-(4-phosphonophenyl)-2-(pyrazolo[1,5-a]pyrimidine-6-carboxamido)acetamido)-3,4-dihydro-2H-benzo[e][1,2]oxaborinine-8-carboxylic acid

The title compound was prepared in a similar manner to the synthesis of Example 186 and collected as the second eluting peak after reversed phase HPLC purification. ESI-MS m/z 566 (M+H)⁺.

### Example 188: Synthesis of (3R)-3-(2-(5-carbamoylpicolinamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 49, utilizing 5-carbamoylpicolinoyl chloride in place of 2-chloro-3,4-dimethoxybenzoyl chloride in Step 3. ESI-MS m/z 569 (M+H)⁺.

### Example 189: Synthesis of (3R)-3-(2-(4-ethyl-2,3-dioxopiperazine-1-carboxamido)-2-(4-sulfophenyl)acetamido)-2-hydroxy-3,4-dihydro-2H-benzo[e][1,2]oxaborinine-8-carboxylic acid

### Step 1: Synthesis of neopentyl 4-formylbenzenesulfonate

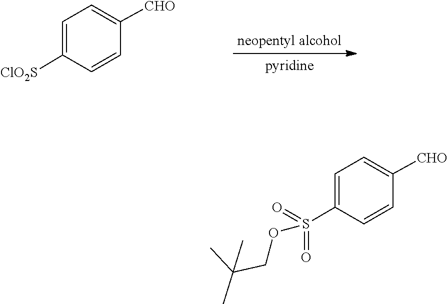 [SMILES: O=Cc1ccc(S(=O)(=O)Cl)cc1 ; CC(C)(C)COS(=O)(=O)c1ccc(C=O)cc1]

To 4-formylbenzenesulfonyl chloride 1 g (4.89 mmol) in chloroform (10 mL) at 0° C. was added pyridine 1.2 mL (14.7 mmol, 3 eq), followed by neopentyl alcohol 0.65 mL (7.33 mmol, 1.5 eq) and the reaction was warmed at RT for 18 h. The mixture was washed with 1N HCl, water, dried over sodium sulfate, and concentrated to dryness to give the title compound, 0.65 g, (51%). ESI-MS m/z 257 (M+H)⁺.

### Step 2: Synthesis of (3R)-3-(2-(4-ethyl-2,3-dioxopiperazine-1-carboxamido)-2-(4-sulfophenyl)acetamido)-2-hydroxy-3,4-dihydro-2H-benzo[e][1,2]oxaborinine-8-carboxylic acid

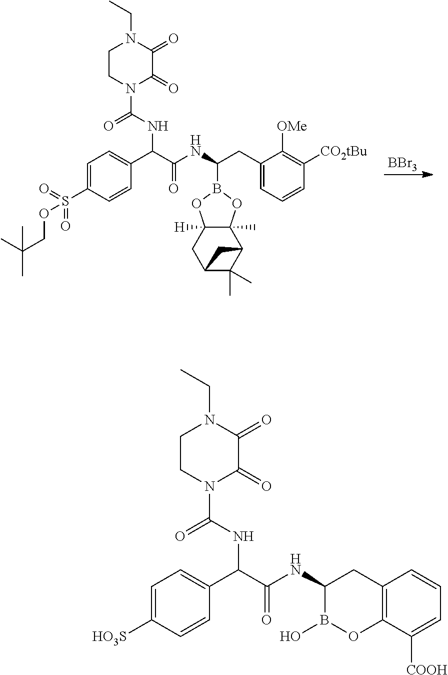 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(S(=O)(=O)OCC(C)(C)C)cc2)C(=O)C1=O ; "BBr3" ; CCN1CCN(C(=O)NC(C(=O)N[C@H]2Cc3cccc(C(=O)O)c3OB2O)c2ccc(S(=O)(=O)O)cc2)C(=O)C1=O]

The title compound was prepared in a similar manner to the synthesis of Example 1, utilizing neopentyl 4-formylbenzenesulfonate in place of 2-fluoro-4-iodobenzaldehyde in Step 1. ESI-MS m/z 589 (M+H)⁺.

### Example 189: Synthesis of (3R)-3-(2-(4-ethyl-2,3-dioxopiperazine-1-carboxamido)-2-(4-sulfophenyl)acetamido)-2-hydroxy-3,4-dihydro-2H-benzo[e][1,2]oxaborinine-8-carboxylic acid

### Step 1: Synthesis of neopentyl 4-formylbenzenesulfonate

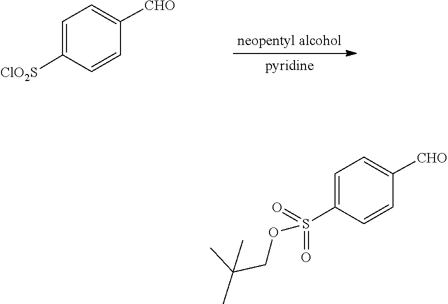 [SMILES: O=Cc1ccc(S(=O)(=O)Cl)cc1 ; CC(C)(C)COS(=O)(=O)c1ccc(C=O)cc1]

To 4-formylbenzenesulfonyl chloride 1 g (4.89 mmol) in chloroform (10 mL) at 0° C. was added pyridine 1.2 mL (14.7 mmol, 3 eq), followed by neopentyl alcohol 0.65 mL (7.33 mmol, 1.5 eq) and the reaction was warmed at RT for 18 h. The mixture was washed with 1N HCl, water, dried over sodium sulfate, and concentrated to dryness to give the title compound, 0.65 g, (51%). ESI-MS m/z 257 (M+H)⁺.

### Step 2: Synthesis of (3R)-3-(2-(4-ethyl-2,3-dioxopiperazine-1-carboxamido)-2-(4-sulfophenyl)acetamido)-2-hydroxy-3,4-dihydro-2H-benzo[e][1,2]oxaborinine-8-carboxylic acid

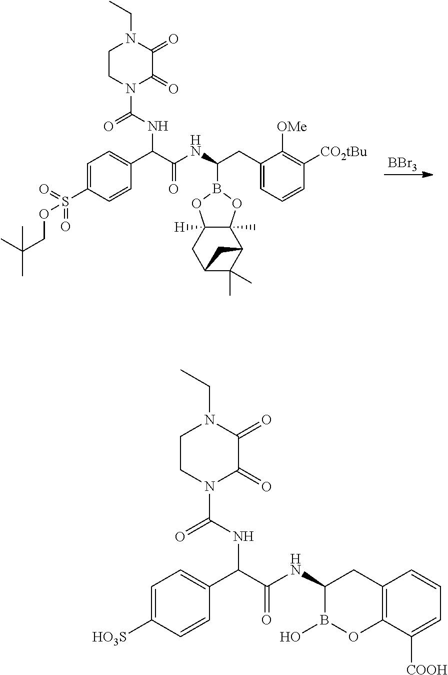 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(S(=O)(=O)OCC(C)(C)C)cc2)C(=O)C1=O ; "BBr3" ; CCN1CCN(C(=O)NC(C(=O)N[C@H]2Cc3cccc(C(=O)O)c3OB2O)c2ccc(S(=O)(=O)O)cc2)C(=O)C1=O]

The title compound was prepared in a similar manner to the synthesis of Example 1, utilizing neopentyl 4-formylbenzenesulfonate in place of 2-fluoro-4-iodobenzaldehyde in Step 1. ESI-MS m/z 589 (M+H)⁺.

### Example 190: Synthesis of (R)-3-((S)-2-(1-(difluoromethyl)-2-oxo-1,2-dihydropyridine-3-carboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 49, utilizing 1-(difluoromethyl)-2-oxo-1,2-dihydropyridine-3-carbonyl chloride in place of 2-chloro-3,4-dimethoxybenzoyl chloride in Step 3. After reversed phase HPLC purification, the title compound was collected as the first eluting peak ESI m/z 592 (M+H)⁺.

### Example 191: Synthesis of (R)-3-((R)-2-(1-(difluoromethyl)-2-oxo-1,2-dihydropyridine-3-carboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 190 and collected as the second eluting peak after reversed phase HPLC purification. ESI-MS m/z 592 (M+H)⁺.

### Example 192: Synthesis of (R)-2-hydroxy-3-((S)-2-(4-phosphonophenyl)-2-(5-(trifluoromethyl)picolinamido)acetamido)-3,4-dihydro-2H-benzo[e][1,2]oxaborinine-8-carboxylic acid

The title compound was prepared in a similar manner to the synthesis of Example 49, utilizing 5-(trifluoromethyl)picolinoyl chloride in place of 2-chloro-3,4-dimethoxybenzoyl chloride in Step 3. After reversed phase HPLC purification, the title compound was collected as the first eluting peak ESI m/z 594 (M+H)⁺.

### Example 193: Synthesis of (R)-2-hydroxy-3-((R)-2-(4-phosphonophenyl)-2-(5-(trifluoromethyl)picolinamido)acetamido)-3,4-dihydro-2H-benzo[e][1,2]oxaborinine-8-carboxylic acid

The title compound was prepared in a similar manner to the synthesis of Example 192 and collected as the second eluting peak after reversed phase HPLC purification. ESI-MS m/z 594 (M+H)⁺.

### Example 194: Synthesis of (R)-2-hydroxy-3-((S)-2-(5-oxo-5H-thiazolo[3,2-a]pyrimidine-6-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing 5-oxo-5H-thiazolo[3,2-a]pyrimidine-6-carbonyl chloride in Step 3. After reversed phase HPLC purification, the title compound was collected as the first eluting peak ESI m/z 599 (M+H)⁺.

### Example 195: Synthesis of (R)-2-hydroxy-3-((R)-2-(5-oxo-5H-thiazolo[3,2-a]pyrimidine-6-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 194 and collected as the second eluting peak after reversed phase HPLC purification. ESI-MS m/z 599 (M+H)⁺.

### Example 196: Synthesis of (R)-2-hydroxy-3-((S)-2-(5-oxo-2,3-dihydro-5H-thiazolo[3,2-a]pyrimidine-6-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 49, utilizing 5-oxo-2,3-dihydro-5H-thiazolo[3,2-a]pyrimidine-6-carbonyl chloride in Step 3. After reversed phase HPLC purification, the title compound was collected as the first eluting peak ESI m z 601 (M+H)⁺.

### Example 197: Synthesis of (R)-2-hydroxy-3-((R)-2-(5-oxo-2,3-dihydro-5H-thiazolo[3,2-a]pyrimidine-6-carboxamido)-2-(4-phosphonophenyl)acetamido)-3,4-dihydro-2H-benzo[e][1,2]oxaborinine-8-carboxylic acid

The title compound was prepared in a similar manner to the synthesis of Example 196 and collected as the second eluting peak after reversed phase HPLC purification. ESI-MS m/z 601 (M+H)⁺.

### Example 198: Synthesis of (3R)-3-(2-(4-ethyl-2,3-dioxopiperazine-1-carboxamido)-2-(3-fluoro-2-hydroxy-4-phosphonophenyl)acetamido)-2-hydroxy-3,4-dihydro-2H-benzo[e][1,2]oxaborinine-8-carboxylic acid

### Step 1: Synthesis of 2-(benzyloxy)-3-fluoro-4-iodobenzaldehyde

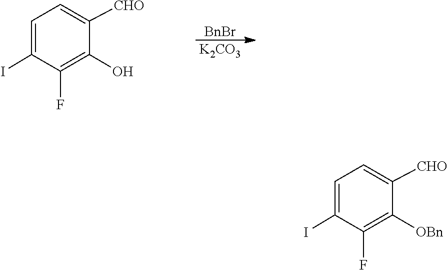

To 3-fluoro-2-hydroxy-4-iodobenzaldehyde 1 g (3.76 mmol) in N, N-dimethylformamide (10 ml) was added potassium carbonate 0.78 g (5.64 mmol, 1.5 eq), followed by benzyl bromide 0.5 mL (4.13 mmol, 1.1 eq) and the reaction was stirred at RT for 1 h. The mixture was diluted with ethyl acetate, washed (3×) with water, dried over sodium sulfate, and concentrated. The product was purified by flash chromatography on silica gel (5% ethyl acetate/hexanes) to give the title compound, 1.17 g, (87%). ESI-MS m/z 357 (M+H)⁺.

### Step 2: Synthesis of (3R)-3-(2-(4-ethyl-2,3-dioxopiperazine-1-carboxamido)-2-(3-fluoro-2-hydroxy-4-phosphonophenyl)acetamido)-2-hydroxy-3,4-dihydro-2H-benzo[e][1,2]oxaborinine-8-carboxylic acid

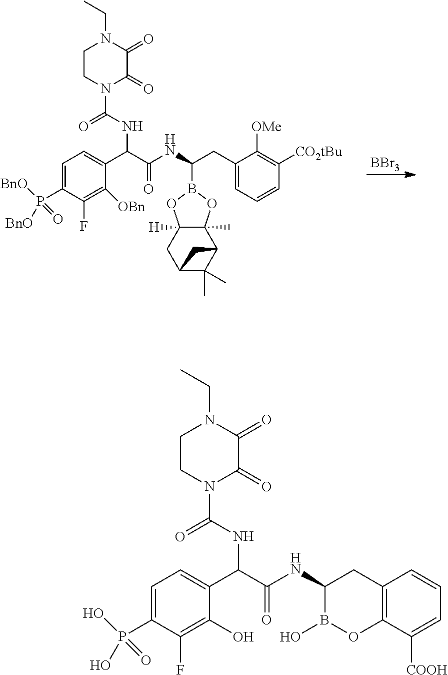 [SMILES: CCN1CCN(C(=O)NC(C(=O)N[C@@H](Cc2cccc(C(=O)OC(C)(C)C)c2OC)B2O[C@@H]3C[C@@H]4C[C@@H](C4(C)C)[C@]3(C)O2)c2ccc(P(=O)(OCc3ccccc3)OCc3ccccc3)c(F)c2OCc2ccccc2)C(=O)C1=O ; "BBr3" ; CCN1CCN(C(=O)NC(C(=O)N[C@H]2Cc3cccc(C(=O)O)c3OB2O)c2ccc(P(=O)(O)O)c(F)c2O)C(=O)C1=O]

The title compound was prepared in a similar manner to the synthesis of Example 1, utilizing 2-(benzyloxy)-3-fluoro-4-iodobenzaldehyde in place of 2-fluoro-4-iodobenzaldehyde in Step 1 and Xantphos Pd G3 in place of tetrakis(triphenylphosphine)palladium(0) in Step 4. ESI-MS m/z 623 (M+H)⁺.

### Example 200: Synthesis of (R)-3-((R)-2-(2-chloro-5-fluoro-3,4-dihydroxyphenyl)-2-(4-(2-(methyl(thiazol-2-ylmethyl)amino)ethyl)-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of tert-butyl(4-fluoro-2-methoxyphenoxy)dimethylsilane

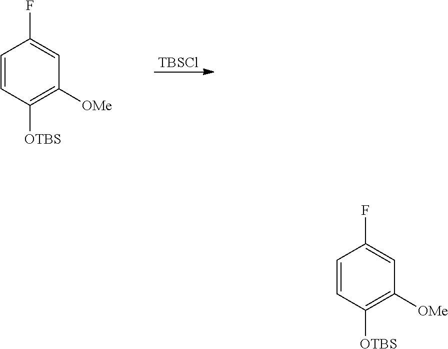 [SMILES: COc1cc(F)ccc1O[Si](C)(C)C(C)(C)C ; "TBSCl" ; COc1cc(F)ccc1O[Si](C)(C)C(C)(C)C]

To a solution of 4-fluoro-2-methoxyphenol (5.68 g, 40 mmol) in DCM (100 mL) was added TEA (11.2 mL, 80 mmol), 4-DMAP (488 mg, 4 mmol) followed by TBSCl (7.5 g, 49.8 mmol). The reaction mixture was stirred at RT for overnight, then re-cooled to 0° C., Boc₂O (36.7 g, 168 mmol) was added. The reaction mixture was stirred at RT overnight, washed with aqueous NaHCO₃, dried over Na₂SO₄, and concentrated. The crude product was purified by flash chromatography on silica gel (hexane-EtOAc, 50:1-10:1) to afford the title compound, 10 g. ESI-MS m/z 257 (M+H)⁺.

### Step 2. Synthesis of tert-butyl 3-((tert-butyldimethylsilyl)oxy)-6-fluoro-2-methoxybenzoate

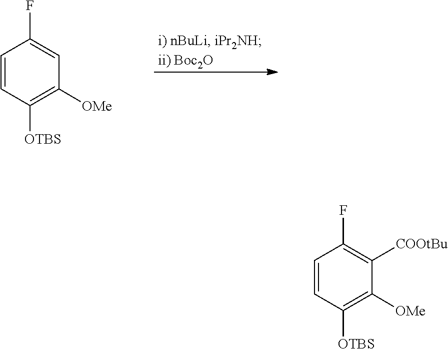

To a solution of diisopropylamine (6.6 mL, 46.8 mmol) in anhydrous THF (120 mL) at −65° C. was added nBuLi (2.5 M, 18.72 mL, 46.8 mmol) dropwise under argon. The reaction mixture was stirred between −60° C. to −55° C. for 20 min. To this reaction mixture was added the above product (10 g, 39 mmol) in THF (15 mL) dropwise, stirred for 1 h, then Boc₂O (28.19 g, 129 mmol) was added. The reaction mixture was slowly warmed up to RT, and stirred at RT overnight, quenched with water, extracted with ethyl acetate. The organic extracts were washed with brine, dried over Na₂SO₄, and concentrated. The crude product was purified by flash chromatography on silica gel (DCM-hexane, 1:20-1:1) to afford the title compound (8 g), which was contaminated with some by product and Boc₂O. ESI-MS m/z 357 (M+H)⁺.

### Step 3. Synthesis of tert-butyl 6-fluoro-3-hydroxy-2-methoxybenzoate

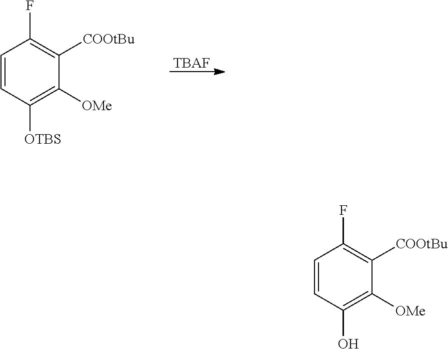

To a solution of the above product (8 g, 22.5 mmol) in THF (150 mL) was added TBAF (1.0 M, 50 mL, 50 mmol), the reaction was stirred at RT for 1.5 h, diluted with EtOAc, washed with saturated aqueous NaHCO₃, brine, dried over Na₂SO₄, and concentrated. The crude product was purified by flash chromatography on silica gel (hexane-EtOAc, 40:1-2:1) to afford the title compound, 1.4 g, ESI-MS m/z 243 (M+H)+, and O-Boc product, 2.6 g, ESI-MS m/z 343 (M+H)⁺. The O-Boc product (2.6 g) was treated with excess piperidine in DCM at RT overnight to afford additional 1.4 g of the title compound after purification by flash chromatography.

### Step 4. Synthesis of tert-butyl 6-fluoro-2-methoxy-3-(((trifluoromethyl)sulfonyl)oxy)benzoate

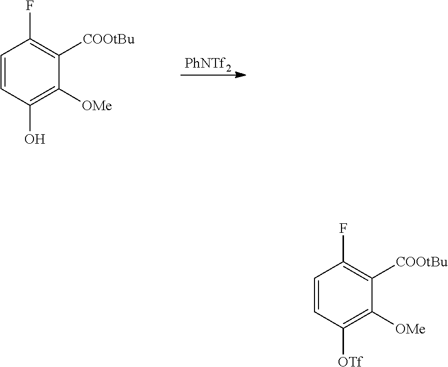

To a solution of the above product (1.4 g, 5.8 mmol) in DCM (50 mL) was added PhNTf₂ (2.9 g, 8.12 mmol), TEA (2.03 mL, 14.5 mmol) and 4-DMAP (71 mg, 0.58 mmol). The reaction mixture was stirred at RT overnight, washed with saturated aqueous NaHCO₃, dried over Na₂SO₄, and concentrated. The crude product was purified by flash chromatography on silica gel (hexane-DCM, 10:1-1:4) to afford the title compound, 1.9 g. ESI-MS m/z 375 (M+Na)⁺.

### Step 5. Synthesis of tert-butyl 6-fluoro-2-methoxy-3-((3aS,4S,6S,7aR)-3a,5,5-trimethylhexahydro-4,6-methanobenzo[d][1,3,2]dioxaborol-2-yl)benzoate

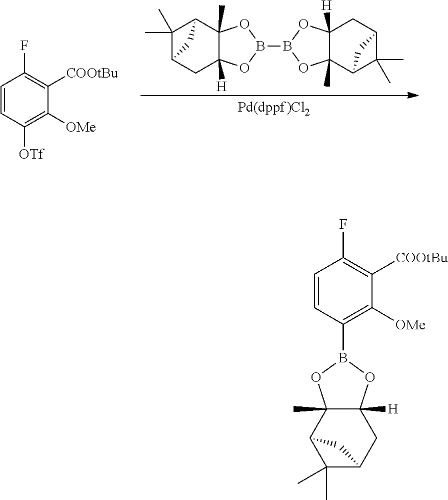

To the above product (3.85 g, 10.3 mmol) in dry DMF (35 mL) was added bis[(+)-pinanediolato]diboron (5.7 g, 15.9 mmol), KOAc (3.1 g, 31.6 mmol) and Pd(dppf)Cl₂·DCM (430 mg, 0.53 mmol). The reaction mixture was stirred at 90-100° C. overnight, added water, and extracted with diethyl ether. The ether extracts were washed with water, brine, dried over Na₂SO₄, and concentrated. The crude product was purified by flash chromatography on silica gel (hexane-DCM, 10:1-1:10) to afford the title compound, 2.4 g. ESI-MS m/z 831 (2M+Na)⁺.

### Step 6. Synthesis of tert-butyl 6-fluoro-2-methoxy-3-(((3aS,4S,6S,7aR)-3a,5,5-trimethylhexahydro-4,6-methanobenzo[d][1,3,2]dioxaborol-2-yl)methyl)benzoate

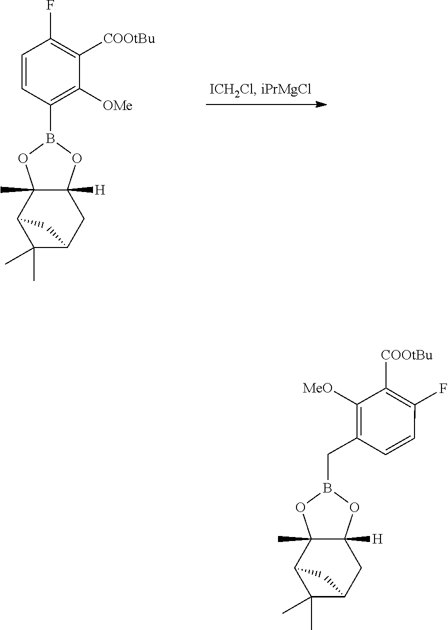

To a solution of chloroiodomethane (3.2 mL, 43.9 mmol) in THF (70 mL) at −78° C. was added dropwise under argon isopropyl magnesium chloride lithium chloride complex solution (1.3 M in THF, 16.8 mL, 21.8 mmol) over 20 min. The resulting solution was stirred at −78° C. for 45 min, then a solution of the above product (2.38 g, 5.89 mmol) in THF (9 mL) was added slowly over 20 min. After the addition was completed, the reaction mixture was stirred for 1.5 h. To this solution was added ZnCl₂ solution (1.0 M in ether, 6.4 mL, 6.4 mmol) dropwise, and stirring continued for 15 min after the addition was completed. The cold bath was removed, the reaction mixture was stirred at RT overnight, cooled to −30° C., diluted with diethyl ether, washed with aqueous NH₄Cl, water and brine, dried over Na₂SO₄, and concentrated, purified by flash chromatography on silica gel (hexane-EtOAc, 20:1-5:1) to give the title compound, 2.24 g. ESI-MS m/z 441 (M+Na)⁺.

### Step 7. Synthesis of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

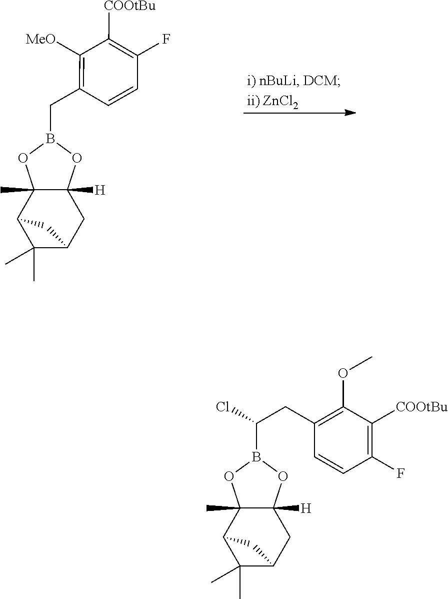

To a cooled (−100° C. MeOH/N₂) solution of DCM (0.82 mL, 12.8 mmol) in THF (15 mL) was added dropwise, down the side of the flask n-BuLi (2.5 M in hexane, 3.06 mL, 7.65 mmol) over 20 min. The resulting mixture was stirred for 45 min, then a solution of the above product (2.24 g, 5.36 mmol) in THF (8 mL) was added slowly down the side of the flask over 20 min, and stirring continued for 45 min after the addition was completed. To the resulting mixture was added dropwise a solution of ZnCl₂ (1.0 M in ether, 7.3 mL, 7.3 mmol) over 5 min. After 15 min, the methanol/N₂ bath was replaced with a dry ice/acetone bath (−10° C.), and stirring continued for 1.5 h. The reaction mixture was diluted with diethyl ether and washed with aqueous NH₄Cl, water and brine, dried over Na₂SO₄, and concentrated. The crude product was purified by flash chromatography on silica gel (hexane-EtOAc, 20:1-4:1) to afford the title compound, 2.1 g. ESI-MS m/z 489 (M+Na)⁺.

### Step 8. Synthesis of 2-chloro-5-fluoro-3,4-dimethoxybenzaldehyde

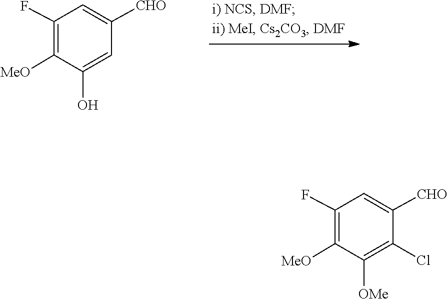 [SMILES: COc1c(O)cc(C=O)cc1F ; COc1c(F)cc(C=O)c(Cl)c1OC]

i) To 3-fluoro-5-hydroxy-4-methoxybenzaldehyde (4.6 g, 27.1 mmol) in DMF (50 mL) was added N-chlorosuccinimide (4.81 g, 36 mmol). The reaction mixture was stirred at RT for 4 h, diluted with diethyl ether, washed with water, brine, dried over Na₂SO₄, concentrated in vacuo to afford the crude product.

ii) To the above crude product (27.1 mmol) in DMF (60 mL) was added Cs₂CO₃ (20.8 g, 63.8 mmol) followed by iodomethane (5 mL, 80 mmol). The reaction mixture was stirred at RT overnight, diluted with diethyl ether, washed with water, brine, dried over Na₂SO₄, concentrated in vacuo. The residue was purified by flash chromatography on silica gel (hexane-EtOAc, 30:1-6:1) to afford the title compound, 1.5 g. ESI-MS m/z 219/221 (M+H)⁺/(M+H+2)⁺. ¹H NMR (400 MHz, CDCl₃) δ 10.36 (s, 1H), 7.48 (d, 1H), 4.1 (s, 3H), 3.19 (br s, 1H), 3.94 (s, 3H).

### Step 9. Synthesis of 2-((tert-butoxycarbonyl)amino)-2-(2-chloro-5-fluoro-3,4-dimethoxyphenyl)acetic acid

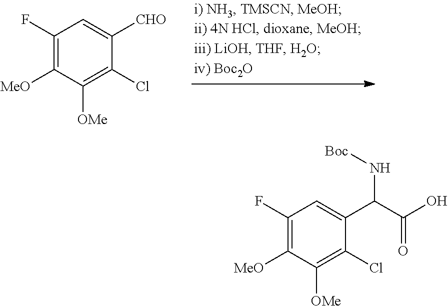 [SMILES: COc1c(F)cc(C=O)c(Cl)c1OC ; COc1c(F)cc(C(NC(=O)OC(C)(C)C)C(=O)O)c(Cl)c1OC]

i) To 2-chloro-5-fluoro-3,4-dimethoxybenzaldehyde (5 g, 22.9 mmol) was added 7 N NH₃ in methanol (200 mL) at 0° C. followed by the addition of TMSCN (5 mL, 40 mmol). The reaction mixture was stirred at 0° C. After 15 min, the reaction mixture was warmed to ˜45° C. After 6 h, the reaction mixture was cooled to ambient temperature and concentrated in vacuo to provide the title compound as a yellow-colored oil which was used without further purification.

ii) This crude product (22.9 mmol) was dissolved in methanol (200 mL) and 4 N HCl in dioxane (200 mL) was added. The reaction mixture was heated at 55-60° C. for 3 days, then concentrated.

iii) To this crude product in THF (180 mL) and water (180 mL) was added LiOH·H₂O (5 g, 119 mmol). The reaction mixture was stirred at RT for 1 h.

iv) This reaction mixture was neutralized with 1 N HCl to pH ˜9, followed by addition of sat. aqueous NaHCO₃ (60 mL) and Boc₂O (8 g, 36.7 mmol) in THF (30 mL) at 0° C. Then the reaction mixture was stirred at RT for 2 h, concentrated, extracted with Et₂O. The aqueous was acidified with 1 N HCl to pH ˜2-3, extracted with EtOAc (2×). The organic extracts were combined, dried over Na₂SO₄, and concentrated in vacuo, yielding the title acid, 7.3 g, ˜85% based on NMR, which was used directly for the next step without further purification. ESI-MS m/z 364/366 (M+H)⁺/(M+H+2)⁺.

### Step 10. Synthesis of tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

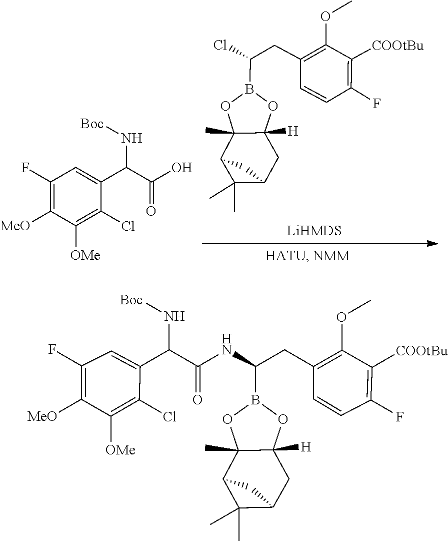

By following the General coupling method C, the title compound was prepared from the above acid and the chloride intermediate from Step 7. ESI-MS m/z 793/795 (M+H)⁺/(M+H+2)⁺.

### Step 11. Synthesis of tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

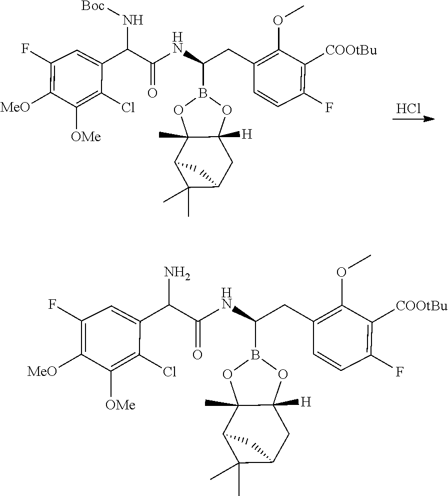

The above compound (11 g, 13.9 mmol) was treated with 2 N HCl in Et₂O (170 mL, 340 mmol) and 4 N HCl in dioxane (88 mL, 352 mmol) at ˜55° C. for 2 h, then concentrated in vacuo, yielding the crude product as HCl salt which was used without further purification for the next step. ESI-MS m/z 693/695 (M+H)⁺/(M+H+2)⁺.

### Step 12. Synthesis of tert-butyl (2-(2,3-dioxopiperazin-1-yl)ethyl)(methyl)carbamate

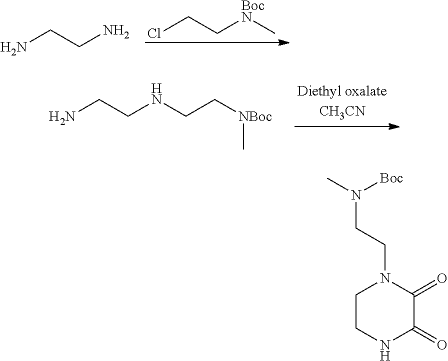

To ethylenediamine (21.88 g, 364 mmol) was added dropwise tert-butyl (2-chloroethyl)(methyl)carbamate (6.984 g, 36 mmol) under argon. The mixture was stirred at room temperature for 27 h and then quenched with brine, extracted with Et₂O (2×), dried over Na₂SO₄, and evaporated under reduced pressure to afford 6.95 g of the title compound. ESI-MS m/z 272 (M+H)⁺.

### Step 13. Synthesis of tert-butyl (2-(4-(chlorocarbonyl)-2,3-dioxopiperazin-1-yl)ethyl)(methyl)carbamate

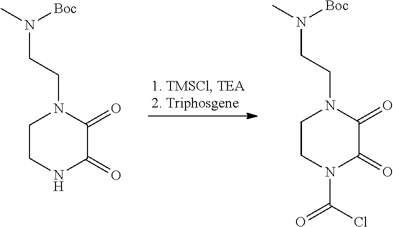 [SMILES: CN(CCN1CCNC(=O)C1=O)C(=O)OC(C)(C)C ; CN(CCN1CCN(C(=O)Cl)C(=O)C1=O)C(=O)OC(C)(C)C]

To a solution of tert-butyl (2-(2,3-dioxopiperazin-1-yl)ethyl)(methyl)carbamate (4 g, 14.8 mmol) in DCM (16 mL) and THF (40 mL) were added dropwise TMSCl (2.1 mL, 16.5 mmol) followed by TEA (2.5 mL, 17.9 mmol) at −15° C. under argon. The reaction mixture was stirred between −15-0° C. for 1 h. A solution of triphosgene (1.8 g, 6.06 mmol) in THF (8 mL) was then added dropwise to the resulting reaction mixture at −15° C. The reaction mixture was allowed to warm to room temperature, stirred at RT for 2 h, and then filtered, washed with THF. The filtrate was evaporated under reduced pressure, dried in vacuo to afford the title compound, which was directly used for the next step.

### Step 14. Synthesis of 3-((2R)-2-(2-(2-chloro-5-fluoro-3,4-dimethoxyphenyl)-2-(4-(2-(methylamino)ethyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoic acid

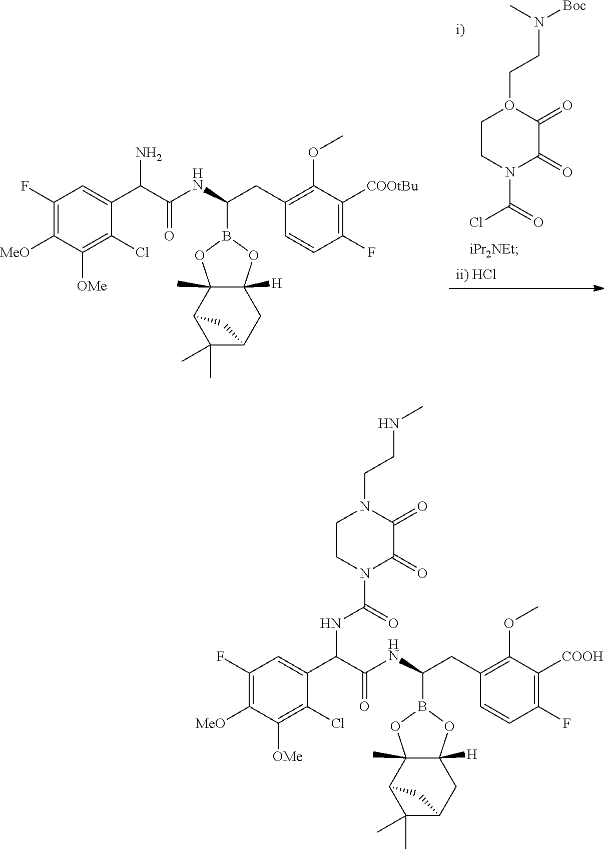

i) To the crude product from Step 11 (2.92 g, 4 mmol) in DCM (100 mL) was added iPr₂NEt (3.2 mL, 18.4 mmol) at 0° C., followed by tert-butyl (2-(4-(chlorocarbonyl)-2,3-dioxopiperazin-1-yl)ethyl)(methyl)carbamate (1.56 g, 4.66 mmol) from Step 13. The reaction mixture was stirred at RT for 1.5 h, washed with water, brine, dried over Na₂SO₄, concentrated in vacuo to yield the crude product which was used without further purification for the next step. ESI-MS m/z 834/836 (M-Boc-^{t}Bu+H)⁺/(MH-Boc-^{t}Bu+2)⁺.

ii) This crude product (4 mmol) was treated with 2 N HCl in Et₂O (56 mL, 112 mmol) and 4 N HCl in dioxane (28 mL, 112 mmol) at RT for 4 h, then concentrated in vacuo, yielding the crude product as HCl salt which was used without further purification for the next step. ESI-MS m/z 834/836 (M+H)⁺/(M+H+2)⁺.

### Step 15. Synthesis of 3-((2R)-2-(2-(2-chloro-5-fluoro-3,4-dimethoxyphenyl)-2-(4-(2-(methyl(thiazol-2-ylmethyl)amino)ethyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoic acid

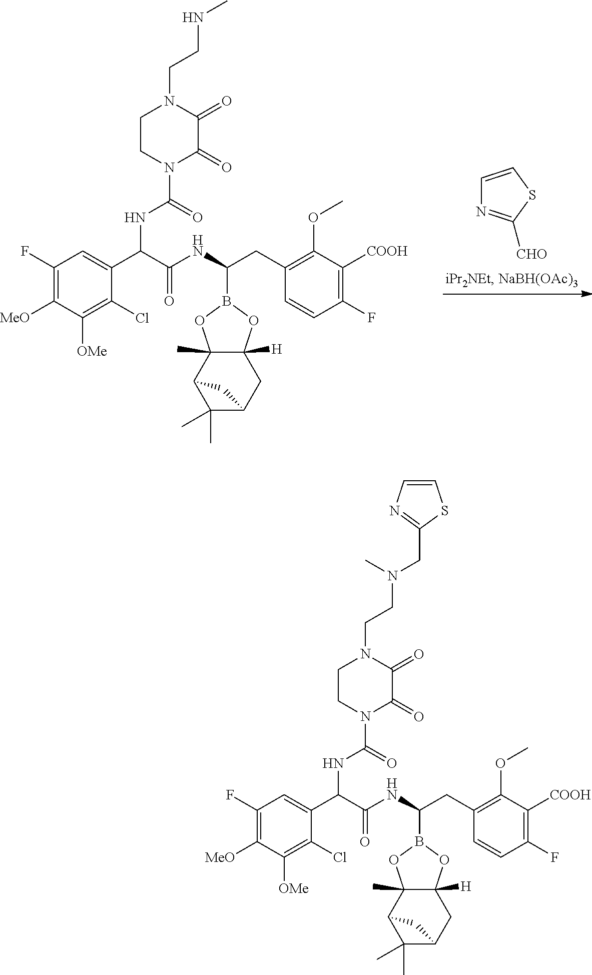 [SMILES: CNCCN1CCN(C(=O)NC(C(=O)N[C@@H](Cc2ccc(F)c(C(=O)O)c2OC)B2O[C@@H]3C[C@@H]4C[C@@H](C4(C)C)[C@]3(C)O2)c2cc(F)c(OC)c(OC)c2Cl)C(=O)C1=O ; O=Cc1nccs1 ; COc1c(F)cc(C(NC(=O)N2CCN(CCN(C)Cc3nccs3)C(=O)C2=O)C(=O)N[C@@H](Cc2ccc(F)c(C(=O)O)c2OC)B2O[C@@H]3C[C@@H]4C[C@@H](C4(C)C)[C@]3(C)O2)c(Cl)c1OC]

To the above crude product (333 mg, 0.38 mmol) in DCE (8 mL) was added iPr₂NEt (0.10 mL, 0.57 mmol), followed by thiazole-2-carboxaldehyde (114 mg, 1 mmol) and NaBH(OAc)₃ (212 mg, 1 mmol). The reaction mixture was stirred at RT for 2 h, diluted with DCM, washed with brine, dried over Na₂SO₄, concentrated in vacuo to yield the crude product which was used without further purification for the next step. ESI-MS m/z 931/933 (M+H)⁺/(M+H+2)⁺.

### Step 16. Synthesis of (R)-3-((R)-2-(2-chloro-5-fluoro-3,4-dihydroxyphenyl)-2-(4-(2-(methyl(thiazol-2-ylmethyl)amino)ethyl)-2,3-dioxopiperazine-1-carboxamido)acetamido)-7-fluoro-2-hydroxy-3,4-dihydro-2H-benzo[e][1,2]oxaborinine-8-carboxylic acid

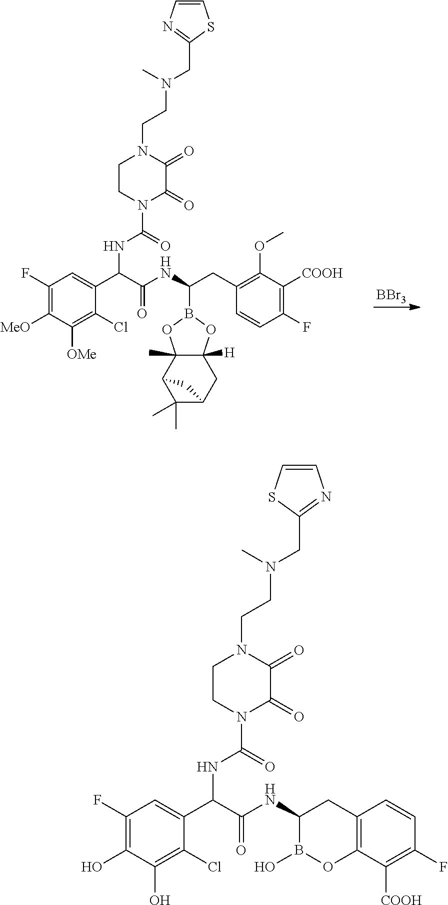 [SMILES: COc1c(F)cc(C(NC(=O)N2CCN(CCN(C)Cc3nccs3)C(=O)C2=O)C(=O)N[C@@H](Cc2ccc(F)c(C(=O)O)c2OC)B2O[C@@H]3C[C@@H]4C[C@@H](C4(C)C)[C@]3(C)O2)c(Cl)c1OC ; "BBr3" ; CN(CCN1CCN(C(=O)NC(C(=O)N[C@H]2Cc3ccc(F)c(C(=O)O)c3OB2O)c2cc(F)c(O)c(O)c2Cl)C(=O)C1=O)Cc1nccs1]

By following the General Method A, the above product was treated with excess BBr₃ at RT overnight, affording the title compound after reversed phase HPLC purification. ESI-MS m/z 737/739 (M+H)⁺/(M+H+2)⁺.

### Example 201: Synthesis of (R)-3-((R)-2-(2-chloro-5-fluoro-3,4-dihydroxyphenyl)-2-(4-(2-(methyl(pyridin-3-ylmethyl)amino)ethyl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 200, utilizing 3-pyridinecarbxaldehyde in place of thiazole-2-carboxaldehyde in Step 15, the title compound was prepared after reversed phase HPLC purification. ESI-MS m/z 731/733 (M+H)⁺/(M+H+2)⁺.

### Example 202: Synthesis of (3R)-3-(2-(4-(3-benzamidopropyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

### Step 1. Synthesis of 3-fluoro-4,5-dihydroxybenzaldehyde

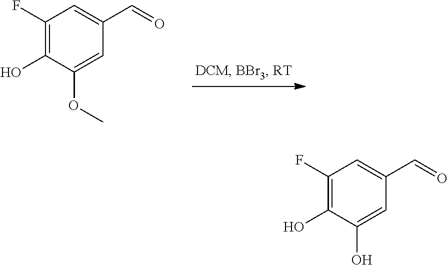

To 3-fluoro-4-hydroxy-5-methoxybenzaldehyde 50 g (294 mmol) in DCM (500 mL) was added a solution of BBr₃ 55 mL (588 mmol, 2 eq) in DCM (250 mL) at −80° C. The reaction mixture was stirred at RT for 6 h. The reaction was quenched by MeOH at −30° C., and evaporated in vacuo. The product was purified by flash chromatography on silica gel (20% MeOH/DCM) to give the desired product, 40 g, 87.3%. ESI-MS m/z 157 (M+H)⁺.

### Step 2. Synthesis of 3-fluoro-5-hydroxy-4-methoxybenzaldehyde

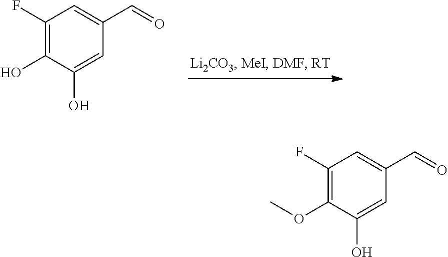

To 3-fluoro-4,5-dihydroxybenzaldehyde 40 g (256 mmol) in DMF (400 mL) was added Li₂CO₃ 28 g (384 mmol, 1.5 eq), followed by MeI 40 g (282 mmol, 1.1 eq) at 0° C. The reaction mixture was stirred at 40° C. for 12 h. The reaction was diluted with EA, washed with NaCl (aq), dried over Na₂SO₄, and evaporated in vacuo. The product was purified by flash chromatography on silica gel (20% EA/PE) to give the desired product, 25 g, 57.5%. ESI-MS m/z 171 (M+H)⁺.

### Step 3: Synthesis of 2-chloro-5-fluoro-3-hydroxy-4-methoxybenzaldehyde

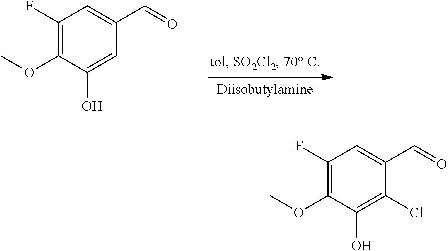

To a solution of 3-fluoro-5-hydroxy-4-methoxybenzaldehyde 25 g (147 mmol) in Toluene was added diisobutylamine 2.1 mL (13 mmol, 0.09 eq). The mixture was heated to 70° C. in oil bath, and added sulfuryl chloride 14 mL (169 mmol, 1.15 eq) at 70° C. The reaction mixture was stirred for 2 h at 70° C. The resulting mixture was concentrated in vacuo, diluted with water, extracted with EA, dried over Na₂SO₄, concentrated in vacuo. The product was purified by flash chromatography on silica gel (10% EA/PE) to give the desired product, 26 g, 86.6%. ESI-MS m/z 205 (M+H)⁺.

### Step 4: Synthesis of 2-chloro-5-fluoro-3,4-dimethoxybenzaldehyde

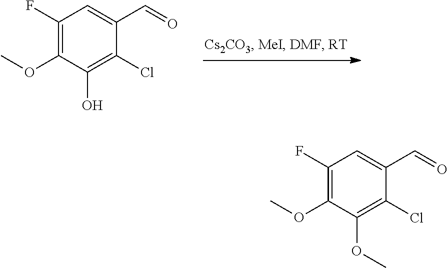

To a solution of 2-chloro-5-fluoro-3-hydroxy-4-methoxybenzaldehyde 26 g (127 mmol) in DMF was added Cs₂CO₃ 62 g (191 mmol, 1.5 eq), followed by MeI 21.6 g (152 mmol, 1.2 eq) at 0° C. The mixture was stirred for 5 h at RT. The reaction mixture was diluted with EA, washed with water, dried over Na₂SO₄, concentrated in vacuo. The product was purified by flash chromatography on silica gel (10% EA/PE) to give the desired product, 25 g, 90.5%. ESI-MS m/z 219 (M+H)⁺.

### Step 5: Synthesis of 2-((tert-butoxycarbonyl)amino)-2-(2-chloro-5-fluoro-3,4-dimethoxyphenyl)acetic acid

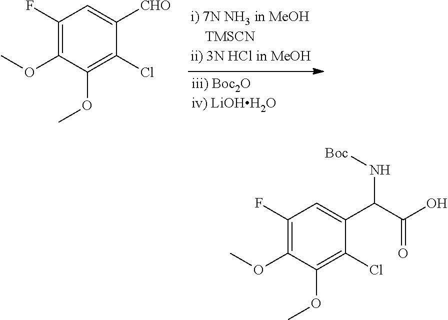 [SMILES: COc1c(F)cc(C=O)c(Cl)c1OC ; COc1c(F)cc(C(NC(=O)OC(C)(C)C)C(=O)O)c(Cl)c1OC]

To 2-chloro-5-fluoro-3,4-dimethoxybenzaldehyde 25 g (115 mmol) at 0° C. was added 7 N ammonia in methanol (550 mL), followed by trimethylsilyl cyanide 21.5 mL (172.5 mmol, 1.5 eq), stirred at 45° C. for 7 h and concentrated in vacuo. The crude product was dissolved in 3 N hydrochloric acid in methanol (450 mL), stirred at 50° C. for 18 h and concentrated in vacuo to give the HCl salt. The reaction was slurried in tetrahydrofuran (500 mL) and cooled at 0° C. Triethylamine 48 mL (345 mmol, 3 eq) was added, followed by di-tert-butyl dicarbonate 37.5 g (172.5 mmol, 1.5 eq), warmed at RT for 1 h and concentrated in vacuo. The product was purified by flash chromatography on silica gel (20-30% ethyl acetate/hexanes) to give the desired product, 30 g. methyl 2-((tert-butoxycarbonyl)amino)-2-(2-chloro-5-fluoro-3,4-dimethoxyphenyl)acetate 30 g (79.5 mmol) was dissolved in tetrahydrofuran (260 mL)/H₂O (260 mL), followed by lithium hydroxide monohydrate 5 g (119 mmol, 1.5 eq) and was stirred at RT for 2 h and concentrated. The product was evaporated in vacuo, adjusted to pH=3 with HCl (1 M), extracted with EA, dried over Na₂SO₄, concentrated in vacuo. The product was purified by flash chromatography on silica gel (40% EA/PE) to give the desired product, 24 g, 83.3%. ESI-MS m/z 364 (M+H)⁺.

### Step 6: Synthesis of tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

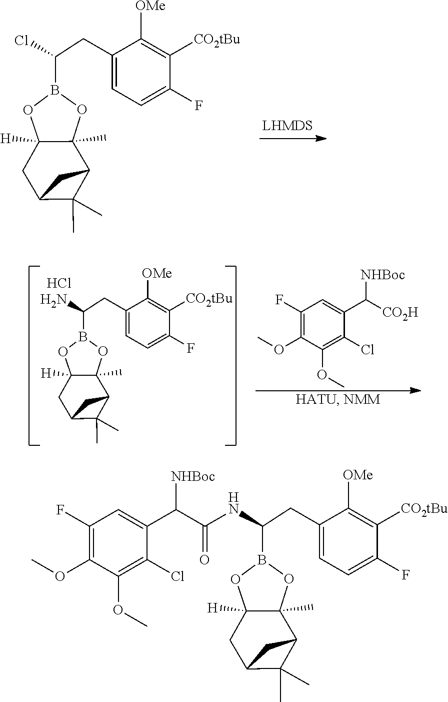

By following the General procedure C, the chloride (prepared as previous reported, WO 2014/089365) was treated with LiHMDS and 4 M HCl in dioxane, and then coupled 2-((tert-butoxycarbonyl)amino)-2-(2-chloro-5-fluoro-3,4-dimethoxyphenyl)acetic acid in the presence of HATU and NMM, yielding the title compound. ESI-MS m/z 793 (M+H)⁺.

### Step 7: Synthesis of tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

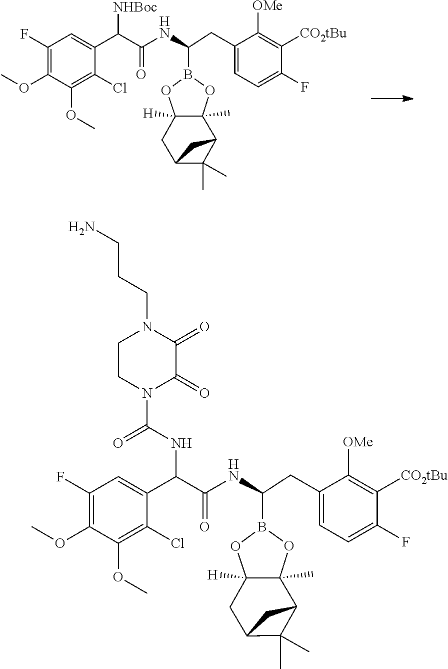 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(CCCN)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

To tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 20 g (25 mmol) at 0° C. was added 1 N hydrochloric acid in diethyl ether (400 mL) and warmed at RT for 18 h. The reaction was concentrated in vacuo to give tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride, 16 g, 91.4%. ESI-MS m/z 693 (M+H)⁺.

To tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 16 g (23 mmol) in dichloromethane (150 mL) at 0° C. was added N,N-diisopropylethylamine 12.7 mL (69 mmol, 3 eq), followed by tert-butyl (3-(4-(chlorocarbonyl)-2,3-dioxopiperazin-1-yl)propyl)carbamate 11.5 g (34.5 mmol, 1.5 eq) and the reaction was warmed at RT for 2 h. The product was quenched with water, washed with brine, dried over sodium sulfate and concentrated to tert-butyl 3-((2R)-2-(2-(4-(3-((tert-butoxycarbonyl)amino)propyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate, 20 g, 88.1%. ESI-MS m/z 990 (M+H)⁺.

The above crude product, 20 g (20 mmol) at 0° C. was added 1 N hydrochloric acid in diethyl ether (400 mL) and warmed at RT for 4h. The reaction was concentrated in vacuo to tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride, 16 g, 89.4%. ESI-MS m/z 890 (M+H)⁺.

### Step 8: Synthesis of (3R)-3-(2-(4-(3-benzamidopropyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

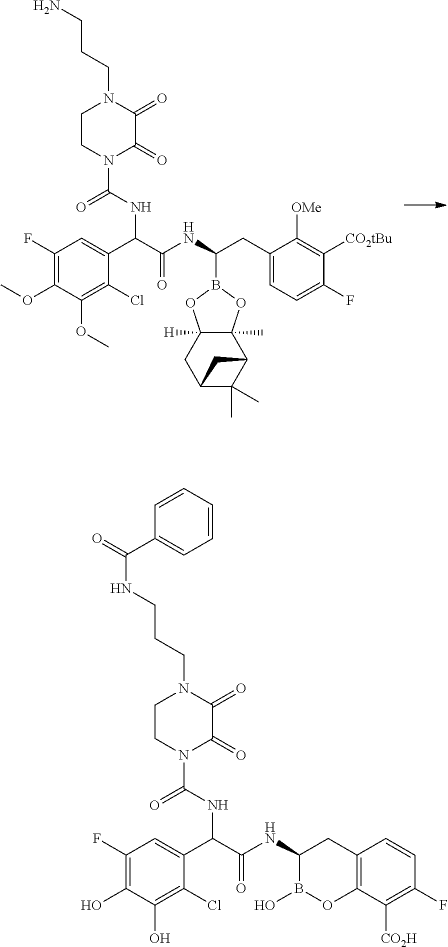 [SMILES: COc1c(F)cc(C(NC(=O)N2CCN(CCCN)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccccc1]

i) To the above crude product, tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride 0.89 g (1 mmol) in dichloromethane (8 mL) was added triethylamine 0.43 mL (3 mmol, 3 eq), followed by benzoyl chloride 0.15 g (1.1 mmol, 1.1 eq) at 0° C. and the reaction mixture was stirred at RT for 18 h. The product was quenched with water, washed with brine, dried over sodium sulfate and concentrated to give the title compound, 0.94 g, 94.7%. ESI-MS m/z 994 (M+H)⁺.

ii) To the above crude product tert-butyl 3-((2R)-2-(2-(4-(3-benzamidopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 0.94 g (0.94 mmol) in dichloromethane at −78° C. was added 1 N boron tribromide in dichloromethane 14.1 mL (14.1 mmol, 15 eq) and warmed at RT for 18 h. The reaction was quenched with water, concentrated and purified on the reverse phase HPLC (5-45% ACN—H₂O+0.1% trifluoroacetic acid to yield the title compound after lyophilization. ESI-MS m/z 744 (M+H)⁺.

### Example 203: Synthesis of (3R)-3-(2-(2-chloro-5-fluoro-3,4-dihydroxyphenyl)-2-(4-(3-(4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 4-methoxybenzoyl chloride in place of benzoyl chloride. ESI-MS m/z 760 (M+H)⁺.

### Example 204: Synthesis of (3R)-3-(2-(2-chloro-5-fluoro-3,4-dihydroxyphenyl)-2-(4-(3-(3-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 3-methoxybenzoyl chloride in place of benzoyl chloride. ESI-MS m/z 760 (M+H)⁺.

### Example 205: Synthesis of (3R)-3-(2-(2-chloro-5-fluoro-3,4-dihydroxyphenyl)-2-(4-(3-(isonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing isonicotinoyl chloride in place of benzoyl chloride. ESI-MS m/z 745 (M+H)⁺.

### Example 206: Synthesis of (3R)-3-(2-(2-chloro-5-fluoro-3,4-dihydroxyphenyl)-2-(4-(3-(3-(3-hydroxyphenyl)ureido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 1-isocyanato-3-methoxybenzene in place of benzoyl chloride. ESI-MS m/z 775 (M+H)⁺.

### Example 207: Synthesis of (3R)-3-(2-(4-(2-benzamidoethyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

### Step 1: Synthesis of tert-butyl 3-((2R)-2-(2-(4-(2-aminoethyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

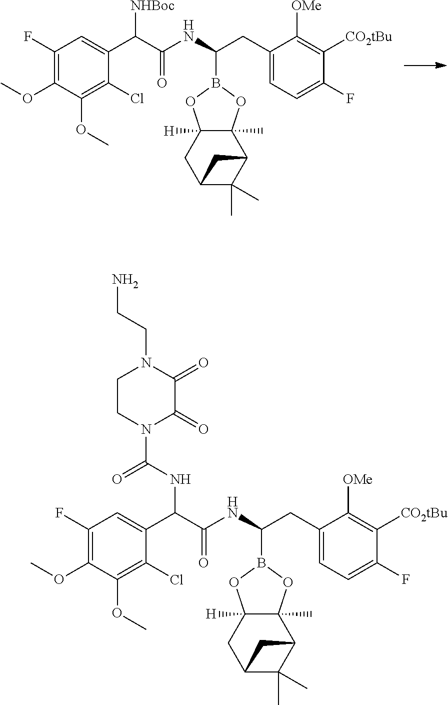 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(CCN)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

The title compound was prepared in a similar manner to the synthesis of Step 7 in Example 202, utilizing tert-butyl (2-(4-(chlorocarbonyl)-2,3-dioxopiperazin-1-yl)ethyl)carbamate in place of tert-butyl (3-(4-(chlorocarbonyl)-2,3-dioxopiperazin-1-yl)propyl)carbamate. ESI-MS m/z 876 (M+H)⁺.

### Step 2: Synthesis of (3R)-3-(2-(4-(2-benzamidoethyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

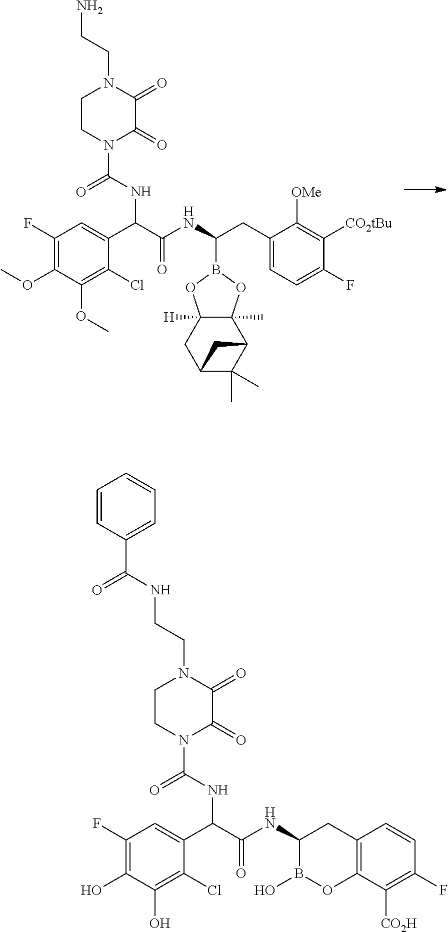 [SMILES: COc1c(F)cc(C(NC(=O)N2CCN(CCN)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)c1ccccc1]

The title compound was prepared in a similar manner to the synthesis of Step 8 in Example 202, utilizing tert-butyl 3-((2R)-2-(2-(4-(2-aminoethyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in place of tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 730 (M+H)⁺.

### Example 208: Synthesis of (R)-3-((R)-2-(2-chloro-5-fluoro-3,4-dihydroxyphenyl)-2-(4-(2-(((5-hydroxypyridin-3-yl)methyl)(methyl)amino)ethyl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 200, utilizing 5-benzloxy-pyridine-3-carbxaldehyde in place of thiazole-2-carboxaldehyde in Step 15, the title compound was prepared after reversed phase HPLC purification. ESI-MS m/z 747/749 (M+H)⁺/(M+H+2)⁺.

### Example 209: Synthesis of (R)-3-((R)-2-(4-(2-(((6-aminopyridin-3-yl)methyl)(methyl)amino)ethyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 200, utilizing tert-butyl (5-formylpyridin-2-yl)carbamate in place of thiazole-2-carboxaldehyde and adding a few drops of HOAc in Step 15, the title compound was prepared after reversed phase HPLC purification. ESI-MS m/z 746/748 (M+H)⁺/(M+H+2)⁺.

### Example 210: Synthesis of (R)-3-((R)-2-(2-chloro-5-fluoro-3,4-dihydroxyphenyl)-2-(4-(3-(nicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing nicotinoyl chloride in place of benzoyl chloride. ESI-MS m/z 745 (M+H)⁺.

### Example 211: Synthesis of (3R)-3-(2-(2-chloro-5-fluoro-3,4-dihydroxyphenyl)-2-(2,3-dioxo-4-(3-(thiazole-2-carboxamido)propyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing thiazole-2-carbonyl chloride in place of benzoyl chloride. ESI-MS m/z 751 (M+H)⁺.

### Example 213: Synthesis of (3R)-3-(2-(2-chloro-5-fluoro-3,4-dihydroxyphenyl)-2-(2,3-dioxo-4-(3-((S)-pyrrolidine-2-carboxamido)propyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing (tert-butoxycarbonyl)-D-proline in place of benzoyl chloride. ESI-MS m/z 737 (M+H)⁺.

### Example 213: Synthesis of (3R)-3-(2-(2-chloro-5-fluoro-3,4-dihydroxyphenyl)-2-(2,3-dioxo-4-(3-(3-(pyridin-3-yl)ureido)propyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 3-isocyanatopyridine in place of benzoyl chloride. ESI-MS m/z 760 (M+H)⁺.

### Example 214: Synthesis of (3R)-3-(2-(2-chloro-5-fluoro-3,4-dihydroxyphenyl)-2-(4-(2-(4-hydroxybenzamido)ethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing 4-methoxybenzoyl chloride in place of benzoyl chloride. ESI-MS m/z 746 (M+H)⁺.

### Example 215: Synthesis of (3R)-3-(2-(2-chloro-5-fluoro-3,4-dihydroxyphenyl)-2-(4-(2-(3-hydroxybenzamido)ethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing 3-methoxybenzoyl chloride in place of benzoyl chloride. ESI-MS m/z 746 (M+H)⁺.

### Example 216: Synthesis of (3R)-3-(2-(2-chloro-5-fluoro-3,4-dihydroxyphenyl)-2-(4-(2-(isonicotinamido)ethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing isonicotinoyl chloride in place of benzoyl chloride. ESI-MS m/z 731 (M+H)⁺.

### Example 217: Synthesis of (3R)-3-(2-(2-chloro-5-fluoro-3,4-dihydroxyphenyl)-2-(4-(2-(nicotinamido)ethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing nicotinoyl chloride in place of benzoyl chloride. ESI-MS m/z 731 (M+H)⁺.

### Example 218: Synthesis of (3R)-3-(2-(2-chloro-5-fluoro-3,4-dihydroxyphenyl)-2-(2,3-dioxo-4-(2-(thiazole-2-carboxamido)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing thiazole-2-carbonyl chloride in place of benzoyl chloride. ESI-MS m/z 737 (M+H)⁺.

### Example 219: Synthesis of (3R)-3-(2-(2-chloro-5-fluoro-3,4-dihydroxyphenyl)-2-(4-(2-(3-(4-hydroxyphenyl)ureido)ethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing 1-isocyanato-4-methoxybenzene in place of benzoyl chloride. ESI-MS m/z 761 (M+H)⁺.

### Example 220: Synthesis of (3R)-3-(2-(2-chloro-5-fluoro-3,4-dihydroxyphenyl)-2-(4-(2-(3-(3-hydroxyphenyl)ureido)ethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing 1-isocyanato-3-methoxybenzene in place of benzoyl chloride. ESI-MS m/z 761 (M+H)⁺.

### Example 221: Synthesis of (R)-3-((R)-2-(2-chloro-5-fluoro-3,4-dihydroxyphenyl)-2-(2,3-dioxo-4-(2-(3-(pyridin-4-yl)ureido)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 208, utilizing 4-isocyanatopyridine in place of benzoyl chloride. ESI-MS m/z 746 (M+H)⁺.

### Example 222: Synthesis of (R)-3-((R)-2-(2-chloro-5-fluoro-3,4-dihydroxyphenyl)-2-(2,3-dioxo-4-(2-(3-(pyridin-3-yl)ureido)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing 3-isocyanatopyridine in place of benzoyl chloride. ESI-MS m/z 746 (M+H)⁺.

### Example 223: Synthesis of (3R)-3-(2-(4-(2-((4-carbamoyl-N-methylphenyl)sulfonamido)ethyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

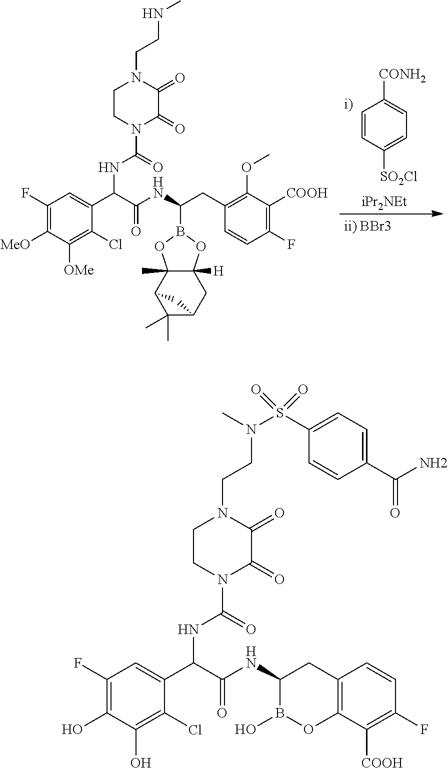 [SMILES: "iPr2NEt" ; CNCCN1CCN(C(=O)NC(C(=O)N[C@@H](Cc2ccc(F)c(C(=O)O)c2OC)B2O[C@@H]3C[C@@H]4C[C@@H](C4(C)C)[C@]3(C)O2)c2cc(F)c(OC)c(OC)c2Cl)C(=O)C1=O ; NC(=O)c1ccc(S(=O)(=O)Cl)cc1 ; CN(CCN1CCN(C(=O)NC(C(=O)N[C@H]2Cc3ccc(F)c(C(=O)O)c3OB2O)c2cc(F)c(O)c(O)c2Cl)C(=O)C1=O)S(=O)(=O)c1ccc(C(N)=O)cc1]

i) To the amine intermediate from Step 14 (333 mg, 0.36 mmol in DCM (100 mL) of Example 200 was added iPr₂NEt (0.32 mL, 1.84 mmol) at 0° C., followed by 4-carbamoylbenzenesulfonyl chloride (99 mg, 0.45 mmol). The reaction mixture was stirred at RT for 1.5 h, washed with water, brine, dried over Na₂SO₄, concentrated in vacuo to yield the crude product which was used without further purification for the next step.

ii) By following the General Method A, this crude product was treated with excess BBr₃ at RT overnight, affording the title compound after reversed phase HPLC purification. ESI-MS m/z 823/825 (M+H)⁺/(M+H+2)⁺.

### Example 224: Synthesis of (3R)-3-(2-(4-(2-((3-carbamoyl-N-methylphenyl)sulfonamido)ethyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 223, utilizing 3-carbamoylbenzenesulfonyl chloride in place of 4-carbamoylbenzenesulfonyl chloride, the title compound was prepared after reversed phase HPLC purification. ESI-MS m/z 823/825 (M+H)⁺/(M+H+2)⁺.

### Example 225: Synthesis of (R)-3-((R)-2-(4-(2-((3-carbamoylbenzyl)(methyl)amino)ethyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 209, utilizing 3-formylbenzamide in place of tert-butyl (5-formylpyridin-2-yl)carbamate, the title compound was prepared after reversed phase HPLC purification. ESI-MS m/z 773/775 (M+H)⁺/(M+H+2)⁺.

### Example 226: Synthesis of (R)-3-((R)-2-(4-(2-((4-carbamoylbenzyl)(methyl)amino)ethyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 209, utilizing 4-formylbenzamide in place of tert-butyl (5-formylpyridin-2-yl)carbamate, the title compound was prepared after reversed phase HPLC purification. ESI-MS m/z 773/775 (M+H)⁺/(M+H+2)⁺.

### Example 227: Synthesis of (3R)-3-(2-(2-chloro-5-fluoro-3,4-dihydroxyphenyl)-2-(4-(3-(3-(4-hydroxyphenyl)ureido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 1-isocyanato-4-methoxybenzene in place of benzoyl chloride. ESI-MS m/z 775 (M+H)⁺.

### Example 228: Synthesis of (3R)-3-(2-(2-chloro-5-fluoro-3,4-dihydroxyphenyl)-2-(2,3-dioxo-4-(3-(3-(pyridin-4-yl)ureido)propyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 4-isocyanatopyridine in place of benzoyl chloride. ESI-MS m/z 760 (M+H)⁺.

### Example 229: Synthesis of (R)-3-((R)-2-(2-chloro-5-fluoro-3,4-dihydroxyphenyl)-2-(2,3-dioxo-4-(2-((S)-pyrrolidine-2-carboxamido)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 207, utilizing (tert-butoxycarbonyl)-D-proline in place of benzoyl chloride. ESI-MS m/z 723 (M+H)⁺.

### Example 230: Synthesis of (3R)-3-(2-(2-chloro-5-fluoro-3,4-dihydroxyphenyl)-2-(4-(2-((4-hydroxyphenyl)amino)-2-oxoethyl)-2,3-dioxopiperazine-1-carboxamido)acetamido)-7-fluoro-2-hydroxy-3,4-dihydro-2H-benzo[e][1,2]oxaborinine-8-carboxylic acid

### Step 1: Synthesis of 2-(4-((2-(((R)-2-(3-(tert-butoxycarbonyl)-4-fluoro-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(2-chloro-5-fluoro-3,4-dimethoxyphenyl)-2-oxoethyl)carbamoyl)-2,3-dioxopiperazin-1-yl)acetic acid

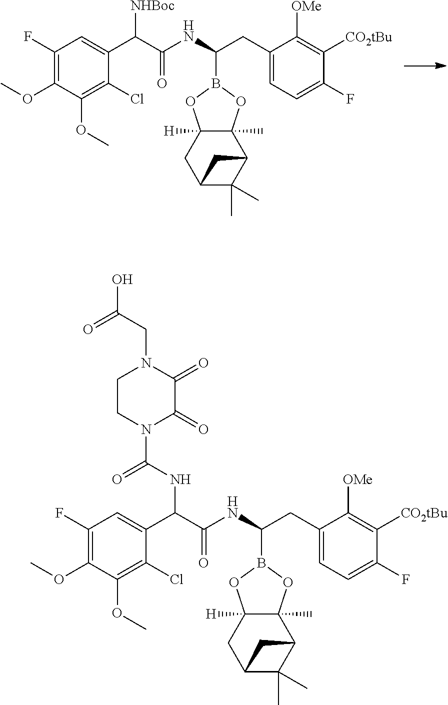 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(CC(=O)O)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

i) To tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 20 g (25 mmol) at 0° C. was added 1 N hydrochloric acid in diethyl ether (400 mL) and warmed at RT for 18 h. The reaction was concentrated in vacuo to give tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride, 16 g, 91.4%. ESI-MS m/z 693 (M+H)⁺.

ii) To tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 16 g (23 mmol) in dichloromethane (150 mL) at 0° C. was added N,N-diisopropylethylamine 12.7 mL (69 mmol, 3 eq), followed by benzyl 2-(4-(chlorocarbonyl)-2,3-dioxopiperazin-1-yl)acetate 11.3 g (34.5 mmol, 1.5 eq) and the reaction was warmed at RT for 2 h. The product was quenched with water, washed with brine, dried over sodium sulfate and concentrated to tert-butyl 3-((2R)-2-(2-(4-(2-(benzyloxy)-2-oxoethyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate, 19 g, 84.1%. ESI-MS m/z 981 (M+H)⁺.

iii) The above crude product, 19 g (19 mmol) was added into a slurry of Pt/C (1.9 g, 10% wt) in MeOH (400 mL). The reaction was stirred for 12 h at RT under H₂. The resulting mixture was filtered through a pad of celite. The filtrate was concentrated in vacuo to 2-(4-((2-(((R)-2-(3-(tert-butoxycarbonyl)-4-fluoro-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(2-chloro-5-fluoro-3,4-dimethoxyphenyl)-2-oxoethyl)carbamoyl)-2,3-dioxopiperazin-1-yl)acetic acid, 15 g, 88.7%. ESI-MS m/z 891 (M+H)⁺.

### Step 8: Synthesis of (3R)-3-(2-(2-chloro-5-fluoro-3,4-dihydroxyphenyl)-2-(4-(2-((4-hydroxyphenyl)amino)-2-oxoethyl)-2,3-dioxopiperazine-1-carboxamido)acetamido)-7-fluoro-2-hydroxy-3,4-dihydro-2H-benzo[e][1,2]oxaborinine-8-carboxylic acid

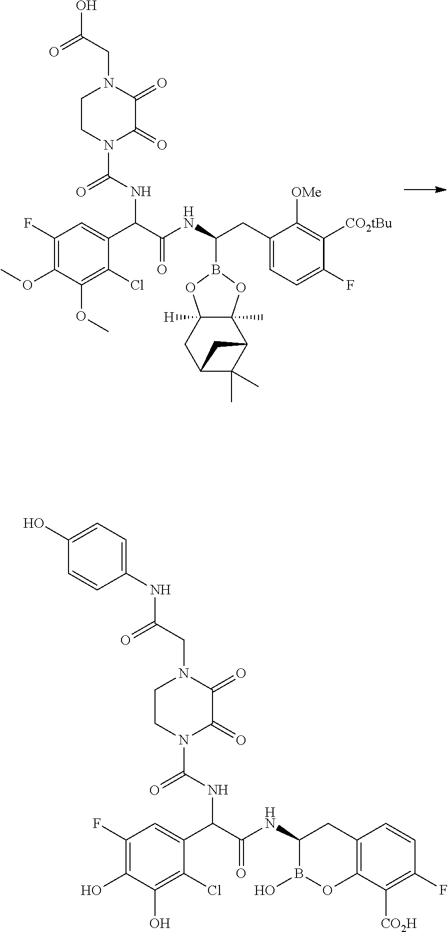 [SMILES: COc1c(F)cc(C(NC(=O)N2CCN(CC(=O)O)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(CN1CCN(C(=O)NC(C(=O)N[C@H]2Cc3ccc(F)c(C(=O)O)c3OB2O)c2cc(F)c(O)c(O)c2Cl)C(=O)C1=O)Nc1ccc(O)cc1]

To the above crude product, 2-(4-((2-(((R)-2-(3-(tert-butoxycarbonyl)-4-fluoro-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(2-chloro-5-fluoro-3,4-dimethoxyphenyl)-2-oxoethyl)carbamoyl)-2,3-dioxopiperazin-1-yl)acetic acid 0.89 g (1 mmol) and 4-methoxyaniline 0.18 g (1.5 mmol, 1.5 eq) in dichloromethane (8 mL) was added triethylamine 0.43 mL (3 mmol, 3 eq), followed by Mukaiyama's reagent 0.51 g (2 mmol, 2 eq) at 0° C. and the reaction mixture was stirred at RT for 18 h. The product was quenched with water, washed with brine, dried over sodium sulfate and concentrated to give the title compound, 0.9 g, 90.3%. ESI-MS m/z 996 (M+H)⁺.

To the above crude product tert-butyl 3-((2R)-2-(2-(2-chloro-5-fluoro-3,4-dimethoxyphenyl)-2-(4-(2-((4-methoxyphenyl)amino)-2-oxoethyl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 0.9 g (0.9 mmol) in dichloromethane at −78° C. was added 1 N boron tribromide in dichloromethane 13.5 mL (13.5 mmol, 15 eq) and warmed at RT for 18 h. The reaction was quenched with water, concentrated and purified on the reverse phase HPLC (5-45% ACN—H₂O+0.1% trifluoroacetic acid to yield the title compound after lyophilization. ESI-MS m/z 732 (M+H)⁺.

### Example 231: Synthesis of (3R)-3-(2-(2-chloro-5-fluoro-3,4-dihydroxyphenyl)-2-(4-(2-((3-hydroxyphenyl)amino)-2-oxoethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing 3-methoxyaniline in place of 4-methoxyaniline. ESI-MS m/z 732 (M+H)⁺.

### Example 232: Synthesis of (3R)-3-(2-(2-chloro-5-fluoro-3,4-dihydroxyphenyl)-2-(2,3-dioxo-4-(2-oxo-2-(phenylamino)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing aniline in place of 4-methoxyaniline. ESI-MS m/z 716 (M+H)⁺.

### Example 233: Synthesis of (3R)-3-(2-(2-chloro-5-fluoro-3,4-dihydroxyphenyl)-2-(2,3-dioxo-4-(2-oxo-2-(pyridin-4-ylamino)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing pyridin-4-amine in place of 4-methoxyaniline. ESI-MS m/z 717 (M+H)⁺.

### Example 234: Synthesis of (3R)-3-(2-(2-chloro-5-fluoro-3,4-dihydroxyphenyl)-2-(2,3-dioxo-4-(2-oxo-2-(pyridin-3-ylamino)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing pyridin-3-amine in place of 4-methoxyaniline. ESI-MS m/z 717 (M+H)⁺.

### Example 235: Synthesis of (3R)-3-(2-(2-chloro-5-fluoro-3,4-dihydroxyphenyl)-2-(2,3-dioxo-4-(2-oxo-2-(thiazol-2-ylamino)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing thiazol-2-amine in place of 4-methoxyaniline. ESI-MS m/z 723 (M+H)⁺.

### Example 236: Synthesis of (3R)-3-(2-(2-chloro-5-fluoro-3,4-dihydroxyphenyl)-2-(4-(2-((4-hydroxybenzyl)amino)-2-oxoethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing (4-methoxyphenyl)methanamine in place of 4-methoxyaniline. ESI-MS m/z 746 (M+H)⁺.

### Example 237: Synthesis of (3R)-3-(2-(2-chloro-5-fluoro-3,4-dihydroxyphenyl)-2-(4-(2-((3-hydroxybenzyl)amino)-2-oxoethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing (3-methoxyphenyl)methanamine in place of 4-methoxyaniline. ESI-MS m/z 746 (M+H)⁺.

### Example 238: Synthesis of (3R)-3-(2-(2-chloro-5-fluoro-3,4-dihydroxyphenyl)-2-(4-(2-((3-hydroxybenzyl)amino)-2-oxoethyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing phenylmethanamine in place of 4-methoxyaniline. ESI-MS m/z 730 (M+H)⁺.

### Example 239: Synthesis of (R)-3-((R)-2-(2-chloro-5-fluoro-3,4-dihydroxyphenyl)-2-(2,3-dioxo-4-(2-oxo-2-((pyridin-4-ylmethyl)amino)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing pyridin-4-ylmethanamine in place of 4-methoxyaniline. ESI-MS m/z 731 (M+H)⁺.

### Example 240: Synthesis of (3R)-3-(2-(2-chloro-5-fluoro-3,4-dihydroxyphenyl)-2-(2,3-dioxo-4-(2-oxo-2-((pyridin-3-ylmethyl)amino)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing pyridin-3-ylmethanamine in place of 4-methoxyaniline. ESI-MS m/z 731 (M+H)⁺.

### Example 241: Synthesis of (R)-3-((R)-2-(2-chloro-5-fluoro-3,4-dihydroxyphenyl)-2-(2,3-dioxo-4-(2-oxo-2-((thiazol-2-ylmethyl)amino)ethyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 230, utilizing thiazol-2-ylmethanamine in place of 4-methoxyaniline. ESI-MS m/z 737 (M+H)⁺.

### Example 242: Synthesis of (3R)-3-(2-(3-(2-benzamidoethyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

### Step 1: Synthesis of tert-butyl 3-((2R)-2-(2-(3-(2-aminoethyl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

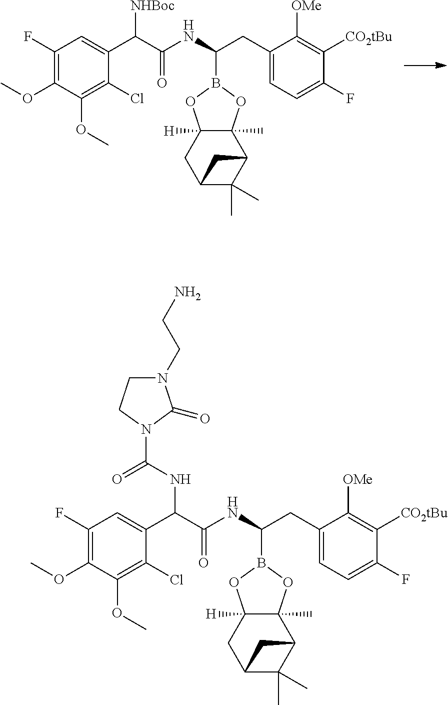 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(CCN)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

The title compound was prepared in a similar manner to the synthesis of Step 7 in Example 202, utilizing tert-butyl (2-(3-(chlorocarbonyl)-2-oxoimidazolidin-1-yl)ethyl)carbamate in place of tert-butyl (3-(4-(chlorocarbonyl)-2,3-dioxopiperazin-1-yl)propyl)carbamate. ESI-MS m/z 848 (M+H)⁺.

### Step 2: Synthesis of (3R)-3-(2-(3-(2-benzamidoethyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

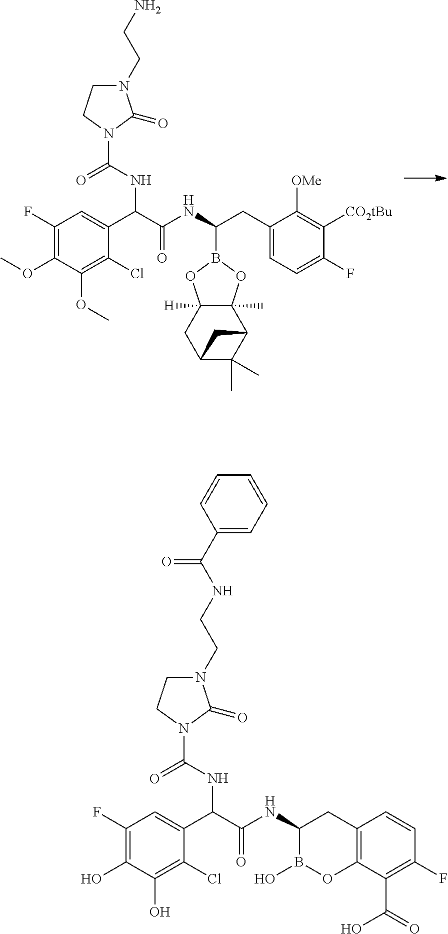 [SMILES: COc1c(F)cc(C(NC(=O)N2CCN(CCN)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(NCCN1CCN(C(=O)NC(C(=O)N[C@H]2Cc3ccc(F)c(C(=O)O)c3OB2O)c2cc(F)c(O)c(O)c2Cl)C1=O)c1ccccc1]

The title compound was prepared in a similar manner to the synthesis of Step 8 in Example 202, utilizing tert-butyl 3-((2R)-2-(2-(3-(2-aminoethyl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in place of tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 702 (M+H)⁺.

### Example 243: Synthesis of (3R)-3-(2-(2-chloro-5-fluoro-3,4-dihydroxyphenyl)-2-(3-(2-(4-hydroxybenzamido)ethyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing 4-methoxybenzoyl chloride in place of benzoyl chloride. ESI-MS m/z 718 (M+H)⁺.

### Example 244: Synthesis of (3R)-3-(2-(2-chloro-5-fluoro-3,4-dihydroxyphenyl)-2-(3-(2-(3-hydroxybenzamido)ethyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing 3-methoxybenzoyl chloride in place of benzoyl chloride. ESI-MS m/z 718 (M+H)⁺.

### Example 245: Synthesis of (3R)-3-(2-(2-chloro-5-fluoro-3,4-dihydroxyphenyl)-2-(3-(2-(isonicotinamido)ethyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing isonicotinoyl chloride in place of benzoyl chloride. ESI-MS m/z 703 (M+H)⁺.

### Example 246: Synthesis of (3R)-3-(2-(2-chloro-5-fluoro-3,4-dihydroxyphenyl)-2-(3-(2-(nicotinamido)ethyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing nicotinoyl chloride in place of benzoyl chloride. ESI-MS m/z 703 (M+H)⁺.

### Example 247: Synthesis of (3R)-3-(2-(2-chloro-5-fluoro-3,4-dihydroxyphenyl)-2-(2-oxo-3-(2-(thiazole-2-carboxamido)ethyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing thiazole-2-carbonyl chloride in place of benzoyl chloride. ESI-MS m/z 709 (M+H)⁺.

### Example 248: Synthesis of (3R)-3-(2-(2-chloro-5-fluoro-3,4-dihydroxyphenyl)-2-(2-oxo-3-(2-((S)-pyrrolidine-2-carboxamido)ethyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing (tert-butoxycarbonyl)-D-proline in place of benzoyl chloride. ESI-MS m/z 695 (M+H)⁺.

### Example 249: Synthesis of (3R)-3-(2-(2-chloro-5-fluoro-3,4-dihydroxyphenyl)-2-(3-(2-(3-(3-hydroxyphenyl)ureido)ethyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing 1-isocyanato-3-methoxybenzene in place of benzoyl chloride. ESI-MS m/z 733 (M+H)⁺.

### Example 250: Synthesis of (3R)-3-(2-(2-chloro-5-fluoro-3,4-dihydroxyphenyl)-2-(2-oxo-3-(2-(3-(pyridin-4-yl)ureido)ethyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing 4-isocyanatopyridine in place of benzoyl chloride. ESI-MS m/z 718 (M+H)⁺.

### Example 251: Synthesis of (3R)-3-(2-(2-chloro-5-fluoro-3,4-dihydroxyphenyl)-2-(2-oxo-3-(2-(3-(pyridin-3-yl)ureido)ethyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing 3-isocyanatopyridine in place of benzoyl chloride. ESI-MS m/z 718 (M+H)⁺.

### Example 252: Synthesis of (R)-3-((S)-2-(3-(3-benzamidopropyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

### Step 1: Synthesis of tert-butyl 3-((2R)-2-(2-(3-(3-aminopropyl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

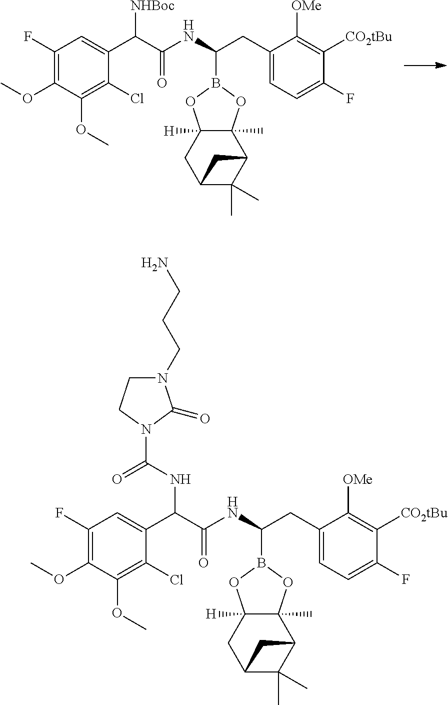 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(CCCN)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

The title compound was prepared in a similar manner to the synthesis of Step 7 in Example 202, utilizing tert-butyl (3-(3-(chlorocarbonyl)-2-oxoimidazolidin-1-yl)propyl)carbamate in place of tert-butyl (3-(4-(chlorocarbonyl)-2,3-dioxopiperazin-1-yl)propyl)carbamate. ESI-MS m/z 862 (M+H)⁺.

### Step 2: Synthesis of (R)-3-((S)-2-(3-(3-benzamidopropyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

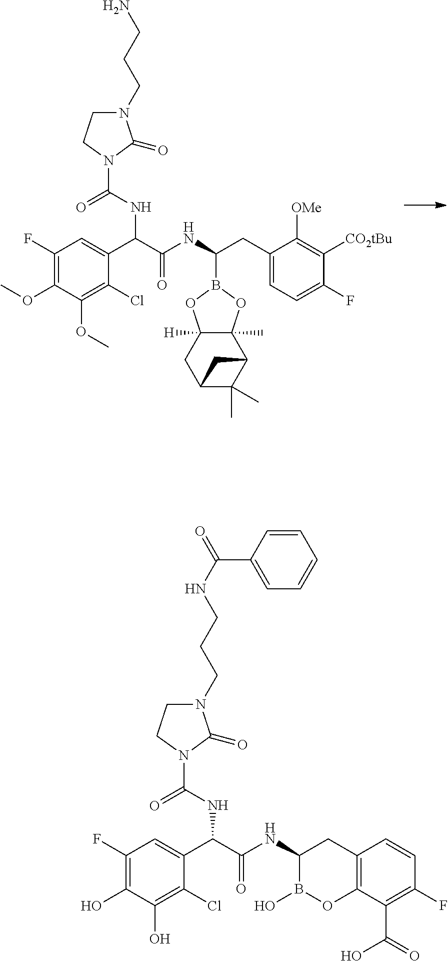 [SMILES: COc1c(F)cc(C(NC(=O)N2CCN(CCCN)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(NCCCN1CCN(C(=O)N[C@H](C(=O)N[C@H]2Cc3ccc(F)c(C(=O)O)c3OB2O)c2cc(F)c(O)c(O)c2Cl)C1=O)c1ccccc1]

The title compound was prepared in a similar manner to the synthesis of Step 8 in Example 203, utilizing tert-butyl 3-((2R)-2-(2-(3-(3-aminopropyl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in place of tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 716 (M+H)⁺.

### Example 253: Synthesis of (R)-3-((R)-2-(3-(3-benzamidopropyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a same manner to the synthesis of Example 252. ESI-MS m/z 716 (M+H)⁺.

### Example 254: Synthesis of (R)-3-((S)-2-(2-chloro-5-fluoro-3,4-dihydroxyphenyl)-2-(3-(3-(3-hydroxybenzamido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing 3-methoxybenzoyl chloride in place of benzoyl chloride. ESI-MS m/z 732 (M+H)⁺.

### Example 255: Synthesis of (R)-3-((R)-2-(2-chloro-5-fluoro-3,4-dihydroxyphenyl)-2-(3-(3-(3-hydroxybenzamido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a same manner to the synthesis of Example 254. ESI-MS m/z 732 (M+H)⁺.

### Example 256: Synthesis of (3R)-3-(2-(2-chloro-5-fluoro-3,4-dihydroxyphenyl)-2-(3-(3-(isonicotinamido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing isonicotinoyl chloride in place of benzoyl chloride. ESI-MS m/z 717 (M+H)⁺.

### Example 258: Synthesis of (R)-3-((R)-2-(2-chloro-5-fluoro-3,4-dihydroxyphenyl)-2-(3-(3-(3-(3-hydroxyphenyl)ureido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing 1-isocyanato-3-methoxybenzene in place of benzoyl chloride. ESI-MS m/z 747 (M+H)⁺.

### Example 259: Synthesis of (3R)-3-(2-(2-chloro-5-fluoro-3,4-dihydroxyphenyl)-2-(2-oxo-3-(3-(3-(pyridin-3-yl)ureido)propyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing 3-isocyanatopyridine in place of benzoyl chloride. ESI-MS m/z 732 (M+H)⁺.

### Example 260: Synthesis of (3R)-3-(2-(3-((2-benzamidoethyl)sulfonyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

### Step 1: Synthesis of tert-butyl 3-((2R)-2-(2-(3-((2-aminoethyl)sulfonyl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

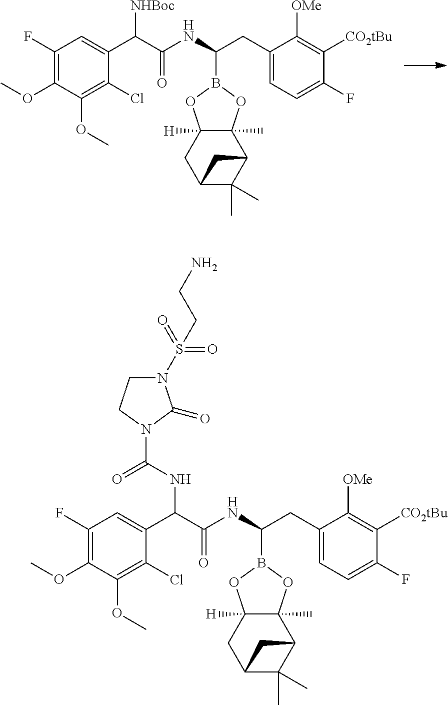 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(S(=O)(=O)CCN)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

The title compound was prepared in a similar manner to the synthesis of Step 7 in Example 202, utilizing tert-butyl (2-((3-(chlorocarbonyl)-2-oxoimidazolidin-1-yl)sulfonyl)ethyl)carbamate in place of tert-butyl (3-(4-(chlorocarbonyl)-2,3-dioxopiperazin-1-yl)propyl)carbamate. ESI-MS m/z 912 (M+H)⁺.

### Step 2: Synthesis of (3R)-3-(2-(3-((2-benzamidoethyl)sulfonyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

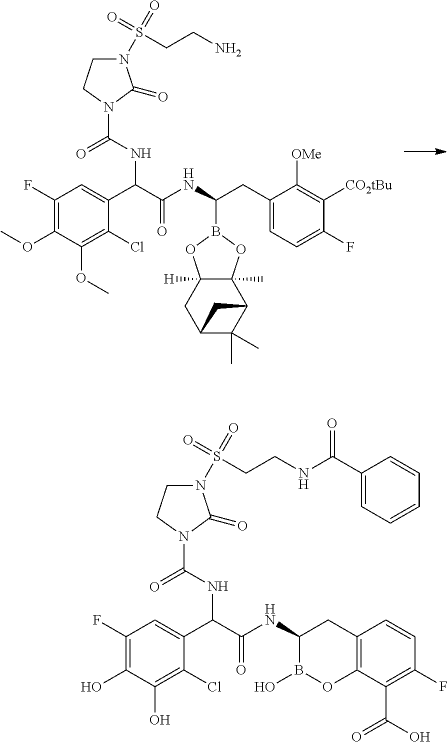 [SMILES: COc1c(F)cc(C(NC(=O)N2CCN(S(=O)(=O)CCN)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(NCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1ccccc1]

The title compound was prepared in a similar manner to the synthesis of Step 8 in Example 202, utilizing tert-butyl 3-((2R)-2-(2-(3-((2-aminoethyl)sulfonyl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in place of tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 766 (M+H)⁺.

### Example 261: Synthesis of (3R)-3-(2-(2-chloro-5-fluoro-3,4-dihydroxyphenyl)-2-(3-((2-(3-hydroxybenzamido)ethyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 260, utilizing 3-methoxybenzoyl chloride in place of benzoyl chloride. ESI-MS m/z 782 (M+H)⁺.

### Example 262: Synthesis of (3R)-3-(2-(2-chloro-5-fluoro-3,4-dihydroxyphenyl)-2-(3-((2-(nicotinamido)ethyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 261, utilizing nicotinoyl chloride in place of benzoyl chloride. ESI-MS m/z 767 (M+H)⁺.

### Example 263: Synthesis of (R)-3-((S)-2-(2-chloro-5-fluoro-3,4-dihydroxyphenyl)-2-(2-oxo-3-((2-(thiazole-2-carboxamido)ethyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 260, utilizing thiazole-2-carbonyl chloride in place of benzoyl chloride. ESI-MS m/z 773 (M+H)⁺.

### Example 264: Synthesis of (R)-3-((R)-2-(2-chloro-5-fluoro-3,4-dihydroxyphenyl)-2-(2-oxo-3-((2-(thiazole-2-carboxamido)ethyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a same manner to the synthesis of Example 263. ESI-MS m/z 773 (M+H)⁺.

### Example 265: Synthesis of (3R)-3-(2-(2-chloro-5-fluoro-3,4-dihydroxyphenyl)-2-(2-oxo-3-((2-((S)-pyrrolidine-2-carboxamido)ethyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 260, utilizing (tert-butoxycarbonyl)-D-proline in place of benzoyl chloride. ESI-MS m/z 759 (M+H)⁺.

### Example 266: Synthesis of (3R)-3-(2-(2-chloro-5-fluoro-3,4-dihydroxyphenyl)-2-(3-((2-(3-(3-hydroxyphenyl)ureido)ethyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 261, utilizing 1-isocyanato-3-methoxybenzene in place of benzoyl chloride. ESI-MS m/z 797 (M+H)⁺.

### Example 267: Synthesis of (R)-3-((R)-2-(2-chloro-5-fluoro-3,4-dihydroxyphenyl)-2-(2-oxo-3-((2-(3-(pyridin-3-yl)ureido)ethyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 260, utilizing 3-isocyanatopyridine in place of benzoyl chloride. ESI-MS m/z 782 (M+H)⁺.

### Example 268: Synthesis of (3R)-3-(2-(3-((3-benzamidopropyl)sulfonyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

### Step 1: Synthesis of tert-butyl 3-((2R)-2-(2-(3-((3-aminopropyl)sulfonyl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

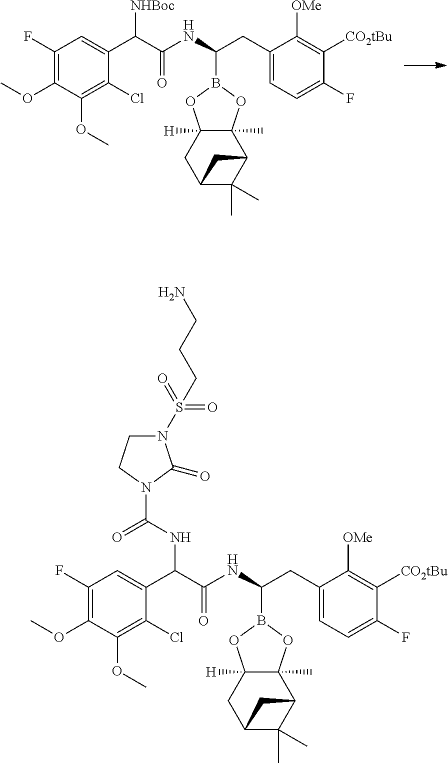 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(S(=O)(=O)CCCN)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

The title compound was prepared in a similar manner to the synthesis of Step 7 in Example 202, utilizing tert-butyl (3-((3-(chlorocarbonyl)-2-oxoimidazolidin-1-yl)sulfonyl)propyl)carbamate in place of tert-butyl (3-(4-(chlorocarbonyl)-2,3-dioxopiperazin-1-yl)propyl)carbamate. ESI-MS m/z 926 (M+H)⁺.

### Step 2: Synthesis of (3R)-3-(2-(3-((3-benzamidopropyl)sulfonyl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

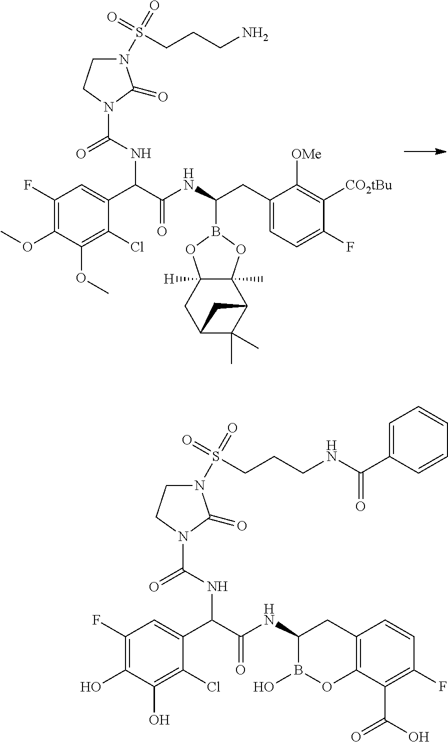 [SMILES: COc1c(F)cc(C(NC(=O)N2CCN(S(=O)(=O)CCCN)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1ccccc1]

The title compound was prepared in a similar manner to the synthesis of Step 8 in Example 202, utilizing tert-butyl 3-((2R)-2-(2-(3-((3-aminopropyl)sulfonyl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in place of tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 780 (M+H)⁺.

### Example 269: Synthesis of (3R)-3-(2-(2-chloro-5-fluoro-3,4-dihydroxyphenyl)-2-(3-((3-(4-hydroxybenzamido)propyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing 4-methoxybenzoyl chloride in place of benzoyl chloride. ESI-MS m/z 796 (M+H)⁺.

### Example 270: Synthesis of (R)-3-((S)-2-(2-chloro-5-fluoro-3,4-dihydroxyphenyl)-2-(3-((3-(3-hydroxybenzamido)propyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing 3-methoxybenzoyl chloride in place of benzoyl chloride. ESI-MS m/z 796 (M+H)⁺.

### Example 271: Synthesis of (R)-3-((R)-2-(2-chloro-5-fluoro-3,4-dihydroxyphenyl)-2-(3-((3-(3-hydroxybenzamido)propyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a same manner to the synthesis of Example 270. ESI-MS m/z 796 (M+H)⁺.

### Example 272: Synthesis of (3R)-3-(2-(2-chloro-5-fluoro-3,4-dihydroxyphenyl)-2-(3-((3-(isonicotinamido)propyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing isonicotinoyl chloride in place of benzoyl chloride. ESI-MS m/z 781 (M+H)⁺.

### Example 273: Synthesis of (3R)-3-(2-(2-chloro-5-fluoro-3,4-dihydroxyphenyl)-2-(3-((3-(nicotinamido)propyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing nicotinoyl chloride in place of benzoyl chloride. ESI-MS m/z 781 (M+H)⁺.

### Example 274: Synthesis of (3R)-3-(2-(2-chloro-5-fluoro-3,4-dihydroxyphenyl)-2-(2-oxo-3-((3-(thiazole-2-carboxamido)propyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing thiazole-2-carbonyl chloride in place of benzoyl chloride. ESI-MS m/z 787 (M+H)⁺.

### Example 275: Synthesis of (3R)-3-(2-(2-chloro-5-fluoro-3,4-dihydroxyphenyl)-2-(2-oxo-3-((3-((S)-pyrrolidine-2-carboxamido)propyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing (tert-butoxycarbonyl)-D-proline in place of benzoyl chloride. ESI-MS m/z 773 (M+H)⁺.

### Example 276: Synthesis of (3R)-3-(2-(2-chloro-5-fluoro-3,4-dihydroxyphenyl)-2-(3-((3-(3-(4-hydroxyphenyl)ureido)propyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing 1-isocyanato-4-methoxybenzene in place of benzoyl chloride. ESI-MS m/z 811 (M+H)⁺.

### Example 277: Synthesis of (3R)-3-(2-(2-chloro-5-fluoro-3,4-dihydroxyphenyl)-2-(3-((3-(3-(3-hydroxyphenyl)ureido)propyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing 1-isocyanato-3-methoxybenzene in place of benzoyl chloride. ESI-MS m/z 811 (M+H)⁺.

### Example 278: Synthesis of (3R)-3-(2-(2-chloro-5-fluoro-3,4-dihydroxyphenyl)-2-(2-oxo-3-((3-(3-(pyridin-3-yl)ureido)propyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing 3-isocyanatopyridine in place of benzoyl chloride. ESI-MS m/z 796 (M+H)⁺.

### Example 279: Synthesis of (R)-3-((R)-2-(2-chloro-5-fluoro-3,4-dihydroxyphenyl)-2-(3-(2-(3-(4-hydroxyphenyl)ureido)ethyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 242, utilizing 1-isocyanato-4-methoxybenzene in place of benzoyl chloride. ESI-MS m/z 733 (M+H)⁺.

### Example 280: Synthesis of (3R)-3-(2-(2-chloro-5-fluoro-3,4-dihydroxyphenyl)-2-(3-(3-(4-hydroxybenzamido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing 4-methoxybenzoyl chloride in place of benzoyl chloride. ESI-MS m/z 732 (M+H)⁺.

### Example 281: Synthesis of (3R)-3-(2-(2-chloro-5-fluoro-3,4-dihydroxyphenyl)-2-(3-(3-(nicotinamido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing nicotinoyl chloride in place of benzoyl chloride. ESI-MS m/z 717 (M+H)⁺.

### Example 282: Synthesis of (3R)-3-(2-(2-chloro-5-fluoro-3,4-dihydroxyphenyl)-2-(3-(3-(nicotinamido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing (tert-butoxycarbonyl)-D-proline in place of benzoyl chloride. ESI-MS m/z 709 (M+H)⁺.

### Example 283: Synthesis of (R)-3-((S)-2-(2-chloro-5-fluoro-3,4-dihydroxyphenyl)-2-(3-(3-(3-(4-hydroxyphenyl)ureido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing 1-isocyanato-4-methoxybenzene in place of benzoyl chloride. ESI-MS m/z 747 (M+H)⁺.

### Example 284: Synthesis of (R)-3-((R)-2-(2-chloro-5-fluoro-3,4-dihydroxyphenyl)-2-(3-(3-(3-(4-hydroxyphenyl)ureido)propyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a same manner to the synthesis of Example 283. ESI-MS m/z 747 (M+H)⁺.

### Example 285: Synthesis of (3R)-3-(2-(2-chloro-5-fluoro-3,4-dihydroxyphenyl)-2-(2-oxo-3-(3-(3-(pyridin-4-yl)ureido)propyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 252, utilizing 4-isocyanatopyridine in place of benzoyl chloride. ESI-MS m/z 732 (M+H)⁺.

### Example 286: Synthesis of (R)-3-((S)-2-(2-chloro-5-fluoro-3,4-dihydroxyphenyl)-2-(3-((2-(4-hydroxybenzamido)ethyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 260, utilizing 4-methoxybenzoyl chloride in place of benzoyl chloride. ESI-MS m/z 782 (M+H)⁺.

### Example 287: Synthesis of (R)-3-((R)-2-(2-chloro-5-fluoro-3,4-dihydroxyphenyl)-2-(3-((2-(4-hydroxybenzamido)ethyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 286. ESI-MS m/z 782 (M+H)⁺.

### Example 288: Synthesis of (3R)-3-(2-(2-chloro-5-fluoro-3,4-dihydroxyphenyl)-2-(3-((2-(isonicotinamido)ethyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 260, utilizing isonicotinoyl chloride in place of benzoyl chloride. ESI-MS m/z 767 (M+H)⁺.

### Example 289: Synthesis of (3R)-3-(2-(2-chloro-5-fluoro-3,4-dihydroxyphenyl)-2-(3-((2-(3-(4-hydroxyphenyl)ureido)ethyl)sulfonyl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 260, utilizing 1-isocyanato-4-methoxybenzene in place of benzoyl chloride. ESI-MS m/z 797 (M+H)⁺.

### Example 290: Synthesis of (R)-3-((R)-2-(2-chloro-5-fluoro-3,4-dihydroxyphenyl)-2-(2-oxo-3-((2-(3-(pyridin-4-yl)ureido)ethyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 260, utilizing 4-isocyanatopyridine in place of benzoyl chloride. ESI-MS m/z 782 (M+H)⁺.

### Example 291: Synthesis of (3R)-3-(2-(2-chloro-5-fluoro-3,4-dihydroxyphenyl)-2-(2-oxo-3-((3-(3-(pyridin-4-yl)ureido)propyl)sulfonyl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 268, utilizing 4-isocyanatopyridine in place of benzoyl chloride. ESI-MS m/z 796 (M+H)⁺.

### Example 292: Synthesis of (3R)-3-(2-(4-(2-chloro-3,4-dihydroxybenzyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

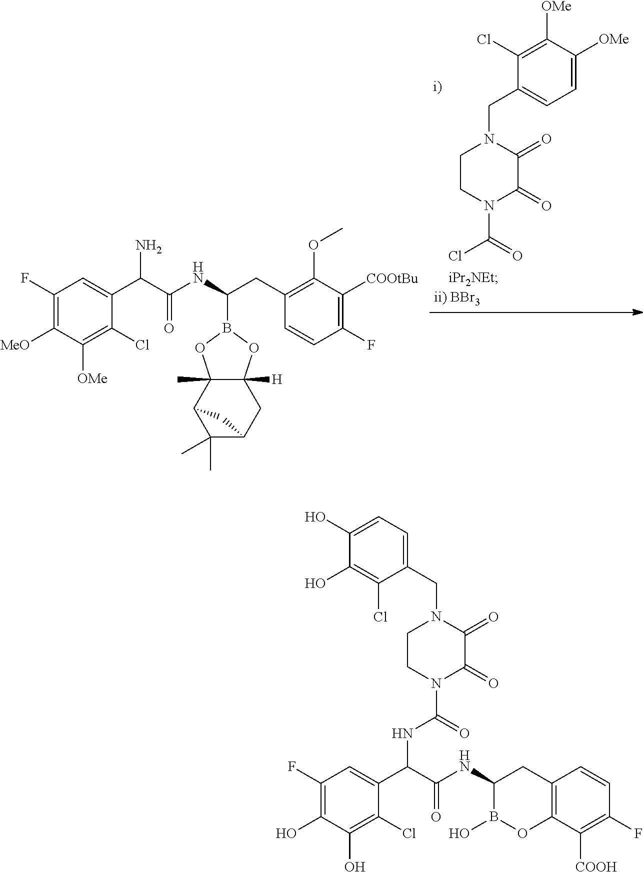

i) To the crude product from Step 11 of Example 200 (291 mg, 0.4 mmol) in DCM (12 mL) was added iPr₂NEt (0.3 mL, 1.72 mmol) at 0° C., followed by 4-(2-chloro-3,4-dimethoxybenzyl)-2,3-dioxopiperazine-1-carbonyl chloride (162 mg, 0.45 mmol). The reaction mixture was stirred at RT for 2 h, washed with water, brine, dried over Na₂SO₄, concentrated in vacuo to yield the crude product which was used without further purification for the next step. ESI-MS m/z 1017/1019/1021 (M+H)⁺/(M+H+2)⁺/(M+H+4)⁺.

ii) By following the General Method A, this crude product was treated with excess BBr₃ at RT overnight, affording the title compound after reversed phase HPLC purification. ESI-MS m/z 739/741/743 (M+H)⁺/(M+H+2)+/(M+H+4)⁺.

### Example 293: Synthesis of (3R)-3-(2-(2-chloro-5-fluoro-3,4-dihydroxyphenyl)-2-(4-(1-(3-hydroxybenzoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of tert-butyl 4-(4-(chlorocarbonyl)-2,3-dioxopiperazin-1-yl)piperidine-1-carboxylate

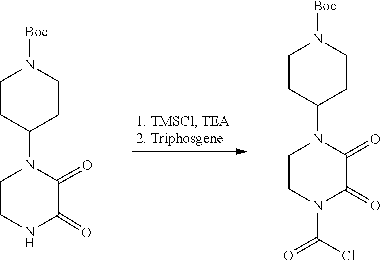 [SMILES: CC(C)(C)OC(=O)N1CCC(N2CCNC(=O)C2=O)CC1 ; CC(C)(C)OC(=O)N1CCC(N2CCN(C(=O)Cl)C(=O)C2=O)CC1]

To a solution of tert-butyl 4-(2,3-dioxopiperazin-1-yl)piperidine-1-carboxylate (4.38 g, 14.7 mmol) in DCM (30 mL) and THF (60 mL) were added dropwise TMSCl (2.1 mL, 16.5 mmol) followed by TEA (2.5 mL, 17.9 mmol) at −15° C. under argon. The reaction mixture was stirred between −15-0° C. for 1 h. A solution of triphosgene (1.8 g, 6.06 mmol) in THF (8 mL) was then added dropwise to the resulting reaction mixture at −15° C. The reaction mixture was allowed to warm to room temperature, stirred at RT for 2 h, and then filtered, washed with THF. The filtrate was evaporated under reduced pressure, dried in vacuo to afford the title compound, which was used for the next step directly.

### Step 2. Synthesis of 3-((2R)-2-(2-(2-chloro-5-fluoro-3,4-dimethoxyphenyl)-2-(2,3-dioxo-4-(piperidin-4-yl)piperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoic acid

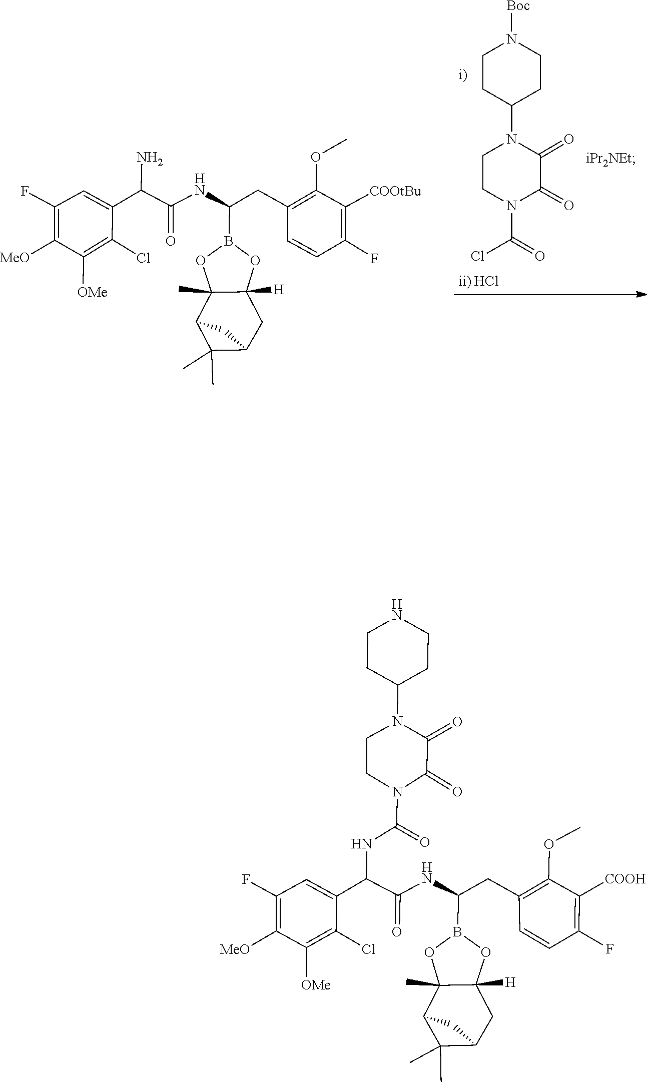

i) To the crude product from Step 11 of Example 200 (3.5 g, 4.8 mmol) in DCM (100 mL) was added iPr₂NEt (3.9 mL, 22.4 mmol) at 0° C., followed by tert-butyl 4-(4-(chlorocarbonyl)-2,3-dioxopiperazin-1-yl)piperidine-1-carboxylate (2.12 g, 5.9 mmol). The reaction mixture was stirred at RT for 2 h, washed with water, brine, dried over Na₂SO₄, concentrated in vacuo to yield the crude product which was used without further purification for the next step. ESI-MS m/z 960/962 (M-^{t}Bu+H)⁺/(M+H-^{t}Bu+2)⁺.

ii) This crude product (4 mmol) was treated with 2 N HCl in Et₂O (68 mL, 136 mmol) and 4 N HCl in dioxane (33 mL, 132 mmol) at RT for 5 h, then concentrated in vacuo, yielding the crude product as HCl salt which was used without further purification for the next step. ESI-MS m/z 860/862 (M+H)⁺/(M+H+2)⁺.

### Step 3. Synthesis of (3R)-3-(2-(2-chloro-5-fluoro-3,4-dihydroxyphenyl)-2-(4-(1-(3-hydroxybenzoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

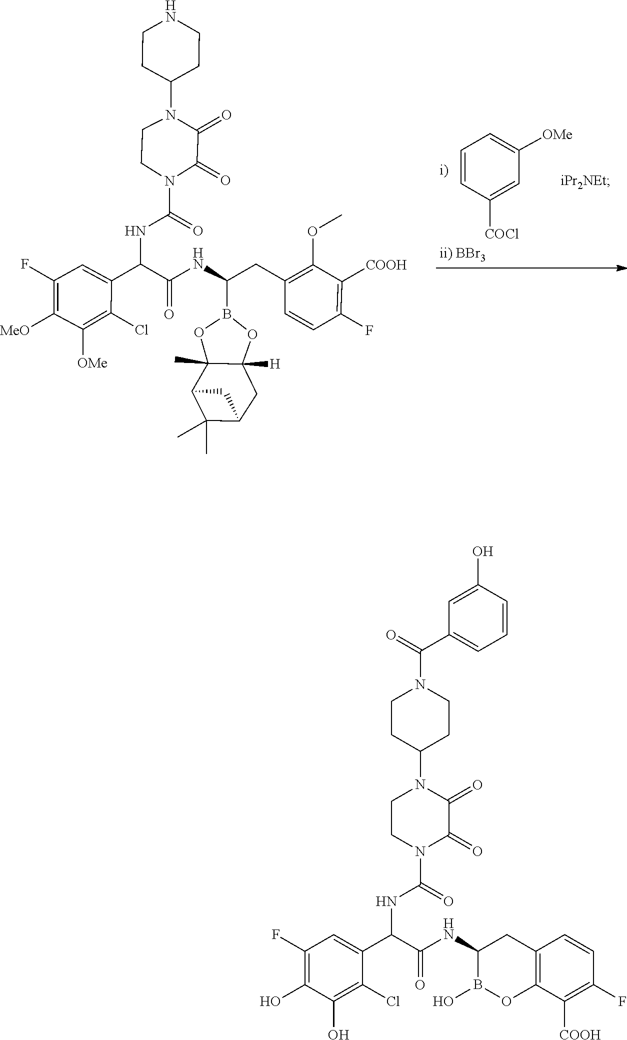 [SMILES: COc1c(F)cc(C(NC(=O)N2CCN(C3CCNCC3)C(=O)C2=O)C(=O)N[C@@H](Cc2ccc(F)c(C(=O)O)c2OC)B2O[C@@H]3C[C@@H]4C[C@@H](C4(C)C)[C@]3(C)O2)c(Cl)c1OC ; COc1cccc(C(=O)Cl)c1 ; O=C(O)c1c(F)ccc2c1OB(O)[C@@H](NC(=O)C(NC(=O)N1CCN(C3CCN(C(=O)c4cccc(O)c4)CC3)C(=O)C1=O)c1cc(F)c(O)c(O)c1Cl)C2]

i) To the above crude amine intermediate (359 mg, 0.4 mmol in DCM (12 mL) was added iPr₂NEt (0.3 mL, 1.71 mmol) at 0° C., followed by 3-methoxybenzoyl chloride (79 mg, 0.46 mmol). The reaction mixture was stirred at RT for 30 min, washed with water, brine, dried over Na₂SO₄, concentrated in vacuo to yield the crude product which was used without further purification for the next step. ii) By following the General Method A, this crude product was treated with excess BBr₃ at RT overnight, affording the title compound after reversed phase HPLC purification. ESI-MS m/z 786/788 (M+H)⁺/(M+H+2)⁺.

### Example 294: Synthesis of (R)-3-((R)-2-(2-chloro-5-fluoro-3,4-dihydroxyphenyl)-2-(4-(3-(isonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing isonicotinoyl chloride in place of benzoyl chloride. ESI-MS m/z 745 (M+H)⁺.

### Example 295: Synthesis of (3R)-3-(2-(2-chloro-5-fluoro-3,4-dihydroxyphenyl)-2-(4-(3-(3-chloroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 3-chloroisonicotinoyl chloride in place of benzoyl chloride. ESI-MS m/z 780 (M+H)⁺.

### Example 296: Synthesis of (R)-3-((R)-2-(2-chloro-5-fluoro-3,4-dihydroxyphenyl)-2-(4-(3-(3-hydroxyisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 3-hydroxyisonicotinoyl chloride in place of benzoyl chloride. ESI-MS m/z 761 (M+H)⁺.

### Example 297: Synthesis of (3R)-3-(2-(2-chloro-5-fluoro-3,4-dihydroxyphenyl)-2-(4-(3-(3-methylisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 3-methylisonicotinoyl chloride in place of benzoyl chloride. ESI-MS m/z 759 (M+H)⁺.

### Example 298: Synthesis of (R)-3-((R)-2-(2-chloro-5-fluoro-3,4-dihydroxyphenyl)-2-(4-(3-(2-methylisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 2-methylisonicotinoyl chloride in place of benzoyl chloride. ESI-MS m/z 759 (M+H)⁺.

### Example 299: Synthesis of (R)-3-((S)-2-(2-chloro-5-fluoro-3,4-dihydroxyphenyl)-2-(4-(3-(2,3-difluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 2,3-difluoroisonicotinoyl chloride in place of benzoyl chloride. ESI-MS m/z 781 (M+H)⁺.

### Example 300: Synthesis of (R)-3-((R)-2-(2-chloro-5-fluoro-3,4-dihydroxyphenyl)-2-(4-(3-(2,3-difluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 299. ESI-MS m/z 781 (M+H)⁺.

### Example 301: Synthesis of (3R)-3-(2-(2-chloro-5-fluoro-3,4-dihydroxyphenyl)-2-(4-(3-(5-fluoro-2-methylisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 5-fluoro-2-methylisonicotinoyl chloride in place of benzoyl chloride. ESI-MS m/z 777 (M+H)⁺.

### Example 302: Synthesis of (R)-3-((S)-2-(4-(3-(2-chloro-3-hydroxybenzamido)propyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 2-chloro-3-methoxybenzoyl chloride in place of benzoyl chloride. ESI-MS m/z 795 (M+H)⁺.

### Example 303: Synthesis of (R)-3-((R)-2-(4-(3-(2-chloro-3-hydroxybenzamido)propyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a same manner to the synthesis of Example 302. ESI-MS m/z 795 (M+H)⁺.

### Example 304: Synthesis of (3R)-3-(2-(2-chloro-5-fluoro-3,4-dihydroxyphenyl)-2-(4-(3-(3-fluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 3-fluoro-4-methoxybenzoyl chloride in place of benzoyl chloride. ESI-MS m/z 778 (M+H)⁺.

### Example 305: Synthesis of (3R)-3-(2-(4-(3-(3-chloro-4-hydroxybenzamido)propyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 3-chloro-4-methoxybenzoyl chloride in place of benzoyl chloride. ESI-MS m/z 795 (M+H)⁺.

### Example 306: Synthesis of (3R)-3-(2-(2-chloro-5-fluoro-3,4-dihydroxyphenyl)-2-(4-(3-(2,3-difluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 2,3-difluoro-4-methoxybenzoyl chloride in place of benzoyl chloride. ESI-MS m/z 796 (M+H)⁺.

### Example 307: Synthesis of (3R)-3-(2-(2-chloro-5-fluoro-3,4-dihydroxyphenyl)-2-(4-(3-(2,4-dihydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 202, utilizing 2,4-dimethoxybenzoyl chloride in place of benzoyl chloride. ESI-MS m/z 776 (M+H)⁺.

### Example 308: Synthesis of (3R)-3-(2-(2-chloro-5-fluoro-3,4-dihydroxyphenyl)-2-(4-(1-(2,6-difluoro-3-hydroxybenzoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 2,6-difluoro-3-methoxybenzoyl chloride (which was prepared by treatment of 2,6-difluoro-3-methoxybenzoic acid with excess oxalyl chloride in DCM) in place of 3-methoxybenzoyl chloride in Step 3, the title compound was prepared after reversed phase HPLC purification. ESI-MS m/z 822/824 (M+H)⁺/(M+H+2)⁺.

### Example 309: Synthesis of (3R)-3-(2-(2-chloro-5-fluoro-3,4-dihydroxyphenyl)-2-(4-(1-(3,5-dihydroxybenzoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 3, 5-dimethoxybenzoyl chloride in place of 3-methoxybenzoyl chloride in Step 3, the title compound was prepared after reversed phase HPLC purification. ESI-MS m/z 802/804 (M+H)⁺/(M+H+2)⁺.

### Example 310: Synthesis of (3R)-3-(2-(2-chloro-5-fluoro-3,4-dihydroxyphenyl)-2-(4-(3-(3-hydroxy-N-methylbenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of tert-butyl (3-(4-(chlorocarbonyl)-2,3-dioxopiperazin-1-yl)propyl)(methyl)carbamate

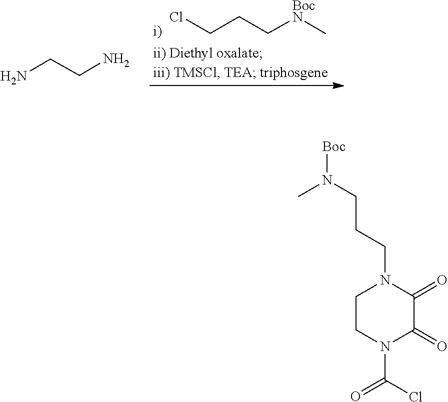

In a similar manner to the synthesis of tert-butyl (2-(4-(chlorocarbonyl)-2,3-dioxopiperazin-1-yl)ethyl)(methyl)carbamate described in Step 12 and 13 of Example 201, utilizing tert-butyl (3-chloropropyl)(methyl)carbamate in place of tert-butyl (2-chloroethyl)(methyl)carbamate, the title compound was prepared.

### Step 2. Synthesis of (3R)-3-(2-(2-chloro-5-fluoro-3,4-dihydroxyphenyl)-2-(4-(3-(3-hydroxy-N-methylbenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

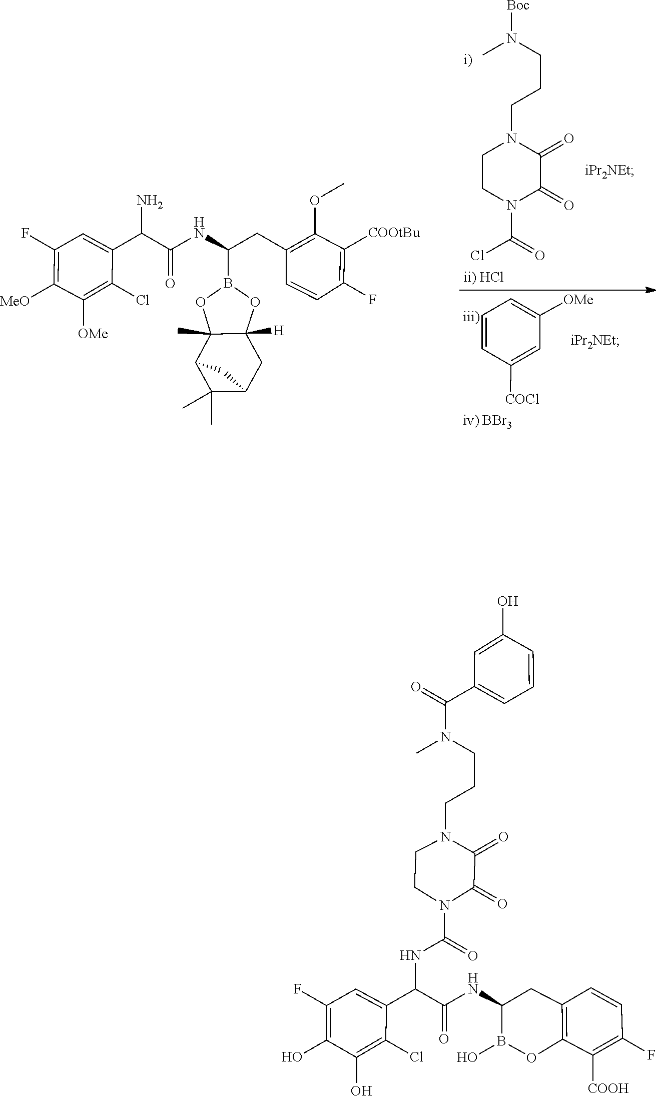

In a similar manner to the synthesis of Example 293, utilizing the above carbamoyl chloride, tert-butyl (3-(4-(chlorocarbonyl)-2,3-dioxopiperazin-1-yl)propyl)(methyl)carbamate prepared in place of tert-butyl 4-(4-(chlorocarbonyl)-2,3-dioxopiperazin-1-yl)piperidine-1-carboxylate in Step 2, the title compound was prepared after reversed phase HPLC purification. ESI-MS m/z 774/776 (M+H)⁺/(M+H+2)⁺.

### Example 400: Synthesis of (3R)-3-(2-(2-chloro-5-fluoro-3,4-dihydroxyphenyl)-2-(4-(3-(3-fluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of 3-fluoro-4,5-dihydroxybenzaldehyde

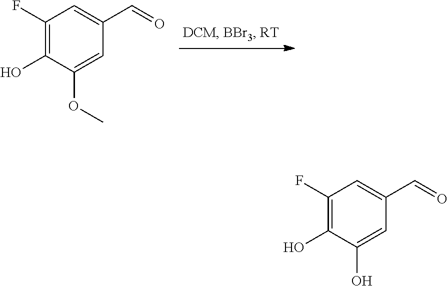

To 3-fluoro-4-hydroxy-5-methoxybenzaldehyde 50 g (294 mmol) in DCM (500 mL) was added a solution of BBr₃ 55 mL (588 mmol, 2 eq) in DCM (250 mL) at −80° C. The reaction mixture was stirred at RT for 6 h. The reaction was quenched by MeOH at −30° C., and evaporated in vacuo. The product was purified by flash chromatography on silica gel (20% MeOH/DCM) to give the desired product, 40 g, 87.3%. ESI-MS m/z 157 (M+H)⁺.

### Step 2. Synthesis of 3-fluoro-5-hydroxy-4-methoxybenzaldehyde

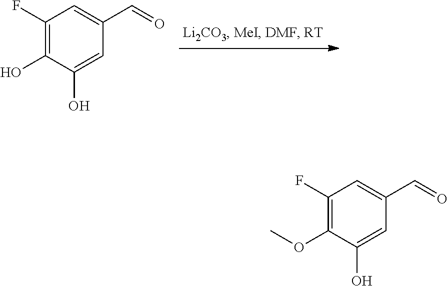

To 3-fluoro-4,5-dihydroxybenzaldehyde 40 g (256 mmol) in DMF (400 mL) was added Li₂CO₃ 28 g (384 mmol, 1.5 eq), followed by MeI 40 g (282 mmol, 1.1 eq) at 0° C. The reaction mixture was stirred at 40° C. for 12 h. The reaction was diluted with EA, washed with NaCl (aq), dried over Na₂SO₄, and evaporated in vacuo. The product was purified by flash chromatography on silica gel (20% EA/PE) to give the desired product, 25 g, 57.5%. ESI-MS m/z 171 (M+H)⁺.

### Step 3: Synthesis of 2-chloro-5-fluoro-3-hydroxy-4-methoxybenzaldehyde

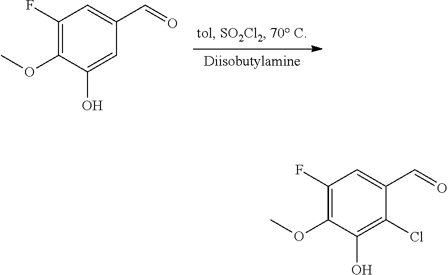

To a solution of 3-fluoro-5-hydroxy-4-methoxybenzaldehyde 25 g (147 mmol) in Toluene was added diisobutylamine 2.1 mL (13 mmol, 0.09 eq). The mixture was heated to 70° C. in oil bath, and added sulfuryl chloride 14 mL (169 mmol, 1.15 eq) at 70° C. The reaction mixture was stirred for 2 h at 70° C. The resulting mixture was concentrated in vacuo, diluted with water, extracted with EA, dried over Na₂SO₄, concentrated in vacuo. The product was purified by flash chromatography on silica gel (10% EA/PE) to give the desired product, 26 g, 86.6%. ESI-MS m/z 205 (M+H)⁺.

### Step 4: Synthesis of 2-chloro-5-fluoro-3,4-dimethoxybenzaldehyde

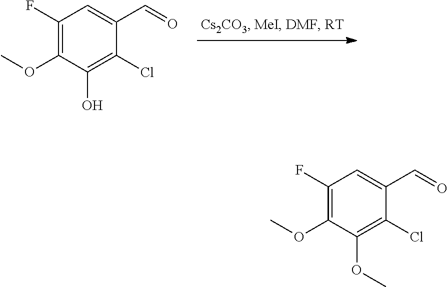

To a solution of 2-chloro-5-fluoro-3-hydroxy-4-methoxybenzaldehyde 26 g (127 mmol) in DMF was added Cs₂CO₃ 62 g (191 mmol, 1.5 eq), followed by MeI 21.6 g (152 mmol, 1.2 eq) at 0° C. The mixture was stirred for 5 h at RT. The reaction mixture was diluted with EA, washed with water, dried over Na₂SO₄, concentrated in vacuo. The product was purified by flash chromatography on silica gel (10% EA/PE) to give the desired product, 25 g, 90.5%. ESI-MS m/z 219 (M+H)⁺.

### Step 5: Synthesis of 2-((tert-butoxycarbonyl)amino)-2-(2-chloro-5-fluoro-3,4-dimethoxyphenyl)acetic acid

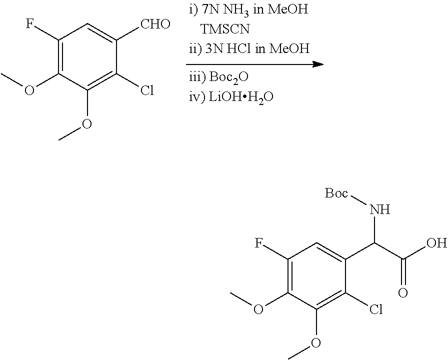 [SMILES: COc1c(F)cc(C=O)c(Cl)c1OC ; COc1c(F)cc(C(NC(=O)OC(C)(C)C)C(=O)O)c(Cl)c1OC]

To 2-chloro-5-fluoro-3,4-dimethoxybenzaldehyde 25 g (115 mmol) at 0° C. was added 7 N ammonia in methanol (550 mL), followed by trimethylsilyl cyanide 21.5 mL (172.5 mmol, 1.5 eq), stirred at 45° C. for 7 h and concentrated in vacuo. The crude product was dissolved in 3N hydrochloric acid in methanol (450 mL), stirred at 50° C. for 18 h and concentrated in vacuo to give the HCl salt. The reaction was slurried in tetrahydrofuran (500 mL) and cooled at 0° C. Triethylamine 48 mL (345 mmol, 3 eq) was added, followed by di-tert-butyl dicarbonate 37.5 g (172.5 mmol, 1.5 eq), warmed at RT for 1 h and concentrated in vacuo. The product was purified by flash chromatography on silica gel (20-30% ethyl acetate/hexanes) to give the desired product, 30 g. methyl 2-((tert-butoxycarbonyl)amino)-2-(2-chloro-5-fluoro-3,4-dimethoxyphenyl)acetate 30 g (79.5 mmol) was dissolved in tetrahydrofuran (260 mL)/H₂O (260 mL), followed by lithium hydroxide monohydrate 5 g (119 mmol, 1.5 eq) and was stirred at RT for 2 h and concentrated. The product was evaporated in vacuo, adjusted to pH=3 with HCl (1 M), extracted with EA, dried over Na₂SO₄, concentrated in vacuo. The product was purified by flash chromatography on silica gel (40% EA/PE) to give the desired product, 24 g, 83.3%. ESI-MS m/z 364 (M+H)⁺.

### Step 6: Synthesis of tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

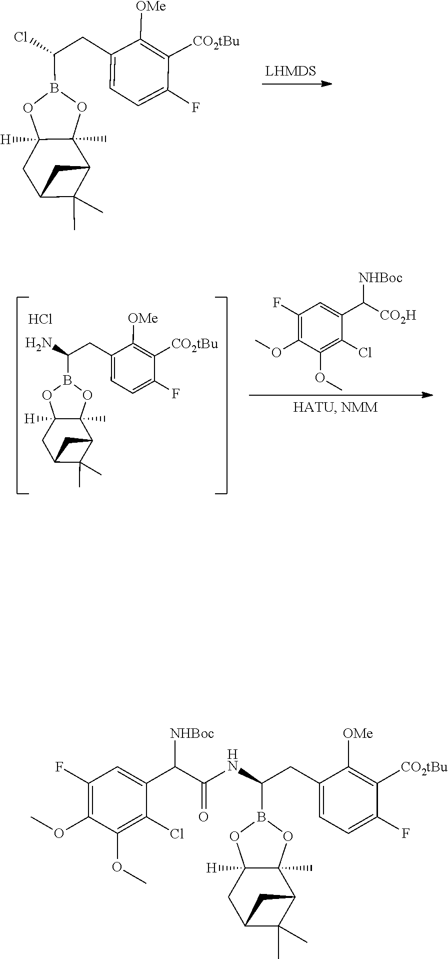

By following the General procedure C, the chloride (prepared as previous reported, WO 2014/089365) was treated with LiHMDS and 4 M dioxane (HCl), and then coupled 2-((tert-butoxycarbonyl)amino)-2-(2-chloro-5-fluoro-3,4-dimethoxyphenyl)acetic acid in the presence of HATU and NMM, yielding the title compound. ESI-MS m/z 793 (M+H)⁺.

### Step 7: Synthesis of tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

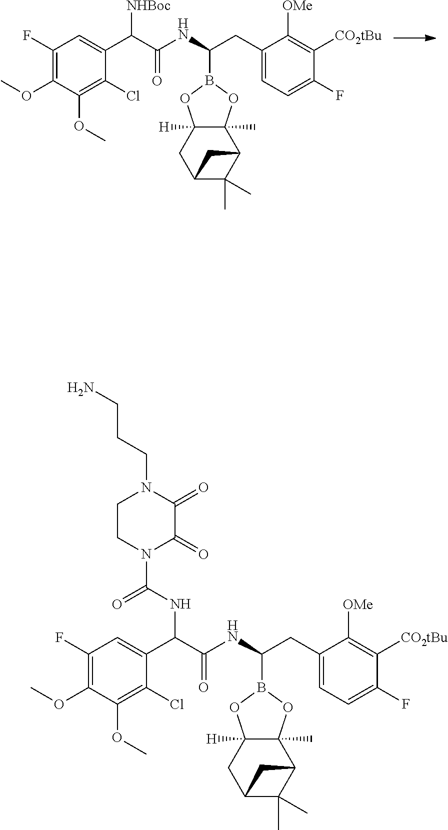 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(CCCN)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

To tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 20 g (25 mmol) at 0° C. was added 1 N hydrochloric acid in diethyl ether (400 mL) and warmed at RT for 18 h. The reaction was concentrated in vacuo to give tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride, 16 g, 91.4%. ESI-MS m/z 693 (M+H)⁺.

To tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 16 g (23 mmol) in dichloromethane (150 mL) at 0° C. was added N,N-diisopropylethylamine 12.7 mL (69 mmol, 3 eq), followed by tert-butyl (3-(4-(chlorocarbonyl)-2,3-dioxopiperazin-1-yl)propyl)carbamate 11.5 g (34.5 mmol, 1.5 eq) and the reaction was warmed at RT for 2 h. The product was quenched with water, washed with brine, dried over sodium sulfate and concentrated to tert-butyl 3-((2R)-2-(2-(4-(3-((tert-butoxycarbonyl)amino)propyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate, 20 g, 88.1%. ESI-MS m/z 990 (M+H)⁺.

The above crude product, 20 g (20 mmol) at 0° C. was added 1 N hydrochloric acid in diethyl ether (400 mL) and warmed at RT for 4h. The reaction was concentrated in vacuo to tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride, 16 g, 89.4%. ESI-MS m/z 890 (M+H)⁺.

### Step 8: Synthesis of (3R)-3-(2-(2-chloro-5-fluoro-3,4-dihydroxyphenyl)-2-(4-(3-(3-fluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

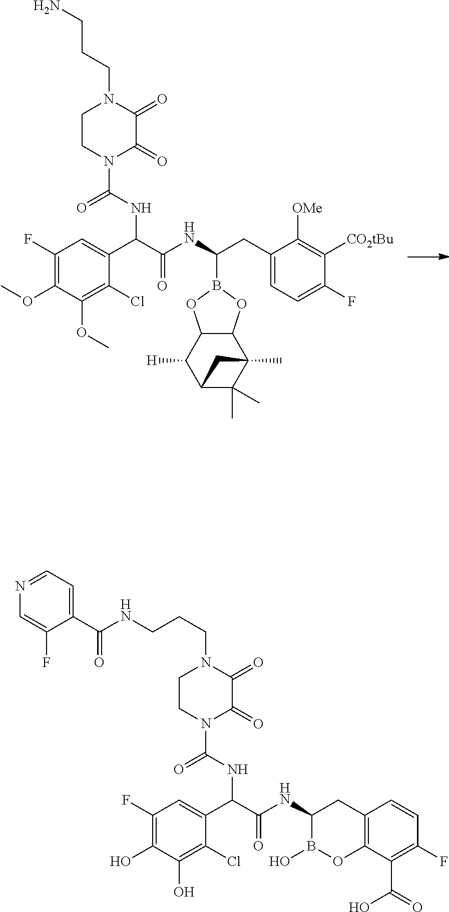

i) To the above crude product, tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride 0.89 g (1 mmol) in dichloromethane (8 mL) was added triethylamine 0.43 mL (3 mmol, 3 eq), followed by 3-fluoroisonicotinoyl chloride 0.175 g (1.1 mmol, 1.1 eq) at 0° C. and the reaction mixture was stirred at RT for 18 h. The product was quenched with water, washed with brine, dried over sodium sulfate and concentrated to give the title compound, 0.91 g, 90%. ESI-MS m/z 1013 (M+H)⁺.

ii) To the above crude product tert-butyl 3-((2R)-2-(2-(4-(3-benzamidopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 0.91 g (0.9 mmol) in dichloromethane at −78° C. was added 1 N boron tribromide in dichloromethane 13.5 mL (13.5 mmol, 15 eq) and warmed at RT for 18 h. The reaction was quenched with water, concentrated, and purified on the reverse phase HPLC (5-45% ACN—H₂O+0.1% trifluoroacetic acid to yield the title compound after lyophilization. ESI-MS m/z 763 (M+H)⁺.

### Example 401: Synthesis of (R)-3-((S)-2-(2-chloro-5-fluoro-3,4-dihydroxyphenyl)-2-(4-(3-(2,6-difluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(4-(3-(2,6-difluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2,6-difluoroisonicotinoyl chloride in place of 3-fluoroisonicotinoyl chloride in Step 8. ESI-MS m/z 781 (M+H)⁺.

### Example 402: Synthesis of (3R)-3-(2-(2-chloro-5-fluoro-3,4-dihydroxyphenyl)-2-(4-(3-(3,5-difluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 3,5-difluoroisonicotinoyl chloride in place of 3-fluoroisonicotinoyl chloride in Step 8. ESI-MS m/z 781 (M+H)⁺.

### Example 403: Synthesis of (3R)-3-(2-(2-chloro-5-fluoro-3,4-dihydroxyphenyl)-2-(4-(3-(3-chloro-5-fluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 3-chloro-5-fluoroisonicotinoyl chloride in place of 3-fluoroisonicotinoyl chloride in Step 8. ESI-MS m/z 797 (M+H)⁺.

### Example 404: Synthesis of (3R)-3-(2-(2-chloro-5-fluoro-3,4-dihydroxyphenyl)-2-(2,3-dioxo-4-(3-(2,4,5-trifluoro-3-hydroxybenzamido)propyl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2,4,5-trifluoro-3-methoxybenzoyl chloride in place of 3-fluoroisonicotinoyl chloride in Step 8. ESI-MS m/z 814 (M+H)⁺.

### Example 405: Synthesis of (R)-3-((S)-2-(2-chloro-5-fluoro-3,4-dihydroxyphenyl)-2-(4-(3-(2,5-difluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid and (R)-3-((S)-2-(2-chloro-5-fluoro-3,4-dihydroxyphenyl)-2-(4-(3-(2,5-difluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2,5-difluoroisonicotinoyl chloride in place of 3-fluoroisonicotinoyl chloride in Step 8. ESI-MS m/z 781 (M+H)⁺.

### Example 406: Synthesis of (3R)-3-(2-(2-chloro-5-fluoro-3,4-dihydroxyphenyl)-2-(4.-(3-(2-fluoro-3-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-fluoro-3-methoxybenzoyl chloride in place of 3-fluoroisonicotinoyl chloride in Step 8. ESI-MS m/z 778 (M+H)⁺.

### Example 407: Synthesis of (3R)-3-(2-(2-chloro-5-fluoro-3,4-dihydroxyphenyl)-2-(4-(3-(2-fluoro-5-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-fluoro-5-methoxybenzoyl chloride in place of 3-fluoroisonicotinoyl chloride in Step 8. ESI-MS m/z 778 (M+H)⁺.

### Example 408: Synthesis of (3R)-3-(2-(2-chloro-5-fluoro-3,4-dihydroxyphenyl)-2-(4-(3-(2-chloro-5-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-chloro-5-methoxybenzoyl chloride in place of 3-fluoroisonicotinoyl chloride in Step 8. ESI-MS m/z 794 (M+H)⁺.

### Example 409: Synthesis of (3R)-3-(2-(2-chloro-5-fluoro-3,4-dihydroxyphenyl)-2-(4-(3-(4-fluoro-3-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 4-fluoro-3-methoxybenzoyl chloride in place of 3-fluoroisonicotinoyl chloride in Step 8. ESI-MS m/z 778 (M+H)⁺.

### Example 410: Synthesis of (3R)-3-(2-(2-chloro-5-fluoro-3,4-dihydroxyphenyl)-2-(4-(3-(3-fluoro-5-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 3-fluoro-5-methoxybenzoyl chloride in place of 3-fluoroisonicotinoyl chloride in Step 8. ESI-MS m/z 778 (M+H)⁺.

### Example 411: Synthesis of (3R)-3-(2-(2-chloro-5-fluoro-3,4-dihydroxyphenyl)-2-(4-(3-(2,4-difluoro-3-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2,4-difluoro-3-methoxybenzoyl chloride in place of 3-fluoroisonicotinoyl chloride in Step 8. ESI-MS m/z 796 (M+H)⁺.

### Example 412: Synthesis of (3R)-3-(2-(4-(3-(2-chloro-4-fluoro-3-hydroxybenzamido)propyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-chloro-4-fluoro-3-methoxybenzoyl chloride in place of 3-fluoroisonicotinoyl chloride in Step 8. ESI-MS m/z 812 (M+H)⁺.

### Example 413: Synthesis of (3R)-3-(2-(2-chloro-5-fluoro-3,4-dihydroxyphenyl)-2-(4-(3-(2,6-difluoro-3-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2,6-difluoro-3-methoxybenzoyl chloride in place of 3-fluoroisonicotinoyl chloride in Step 8. ESI-MS m/z 796 (M+H)⁺.

### Example 414: Synthesis of (3R)-3-(2-(2-chloro-5-fluoro-3,4-dihydroxyphenyl)-2-(4-(3-(2-chloro-6-fluoro-3-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-chloro-6-fluoro-3-methoxybenzoyl chloride in place of 3-fluoroisonicotinoyl chloride in Step 8. ESI-MS m/z 812 (M+H)⁺.

### Example 415: Synthesis of (3R)-3-(2-(2-chloro-5-fluoro-3,4-dihydroxyphenyl)-2-(4-(3-(2,4-difluoro-3,5-dihydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2,4-difluoro-3,5-dimethoxybenzoyl chloride in place of 3-fluoroisonicotinoyl chloride in Step 8. ESI-MS m/z 812 (M+H)⁺.

### Example 416: Synthesis of (3R)-3-(2-(2-chloro-5-fluoro-3,4-dihydroxyphenyl)-2-(4-(3-(4-fluoro-3,5-dihydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 4-fluoro-3,5-dimethoxybenzoyl chloride in place of 3-fluoroisonicotinoyl chloride in Step 8. ESI-MS m/z 794 (M+H)⁺.

### Example 417: Synthesis of (R)-3-((S)-2-(2-chloro-5-fluoro-3,4-dihydroxyphenyl)-2-(4-(3-(2-fluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(4-(3-(2-fluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-fluoro-4-methoxybenzoyl chloride in place of 3-fluoroisonicotinoyl chloride in Step 8. ESI-MS m/z 778 (M+H)⁺.

### Example 418: Synthesis of (3R)-3-(2-(4-(3-(2-chloro-3-fluoro-4-hydroxybenzamido)propyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-chloro-3-fluoro-4-methoxybenzoyl chloride in place of 3-fluoroisonicotinoyl chloride in Step 8. ESI-MS m/z 812 (M+H)⁺.

### Example 419: Synthesis of (3R)-3-(2-(4-(3-(3-chloro-2-fluoro-4-hydroxybenzamido)propyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 3-chloro-2-fluoro-4-methoxybenzoyl chloride in place of 3-fluoroisonicotinoyl chloride in Step 8. ESI-MS m/z 812 (M+H)⁺.

### Example 420: Synthesis of (R)-3-((S)-2-(2-chloro-5-fluoro-3,4-dihydroxyphenyl)-2-(4-(3-(2,6-difluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(4-(3-(2,6-difluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2,6-difluoro-4-methoxybenzoyl chloride in place of 3-fluoroisonicotinoyl chloride in Step 8. ESI-MS m/z 796 (M+H)⁺.

### Example 421: Synthesis of (3R)-3-(2-(2-chloro-5-fluoro-3,4-dihydroxyphenyl)-2-(4-(3-(2-chloro-6-fluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-chloro-6-fluoro-4-methoxybenzoyl chloride in place of 3-fluoroisonicotinoyl chloride in Step 8. ESI-MS m/z 812 (M+H)⁺.

### Example 422: Synthesis of (R)-3-((R)-2-(2-chloro-5-fluoro-3,4-dihydroxyphenyl)-2-(4-(3-(3,5-difluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 3,5-difluoro-4-methoxybenzoyl chloride in place of 3-fluoroisonicotinoyl chloride in Step 8. ESI-MS m/z 796 (M+H)⁺.

### Example 423: Synthesis of (3R)-3-(2-(2-chloro-5-fluoro-3,4-dihydroxyphenyl)-2-(4-(3-(3,5-dihydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 3,5-dimethoxybenzoyl chloride in place of 3-fluoroisonicotinoyl chloride in Step 8. ESI-MS m/z 776 (M+H)⁺.

### Example 424: Synthesis of (3R)-3-(2-(2-chloro-5-fluoro-3,4-dihydroxyphenyl)-2-(4-(1-((3-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 1-isocyanato-3-methoxybenzene in place of 3-methoxybenzoyl chloride in Step 3, the title compound was prepared after reversed phase HPLC purification. ESI-MS m/z 801/803 (M+H)⁺/(M+H+2)⁺.

### Example 425: Synthesis of (3R)-3-(2-(2-chloro-5-fluoro-3,4-dihydroxyphenyl)-2-(4-(1-(5-hydroxynicotinoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 5-(benzyloxy)nicotinoyl chloride (which was made from 5-(benzyloxy)nicotinic acid by treatment with excess of oxallyl chloride in DCM at RT) in place of 3-methoxybenzoyl chloride in Step 3, the title compound was prepared after reversed phase HPLC purification. ESI-MS m/z 787/789 (M+H)⁺/(M+H+2)⁺.

### Example 426: Synthesis of (R)-3-((S)-2-(2-chloro-5-fluoro-3,4-dihydroxyphenyl)-2-(4-(1-((4-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(4-(1-((4-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

### Step 1. Synthesis of tert-butyl 4-(3-(chlorocarbonyl)-2-oxoimidazolidin-1-yl)piperidine-1-carboxylate

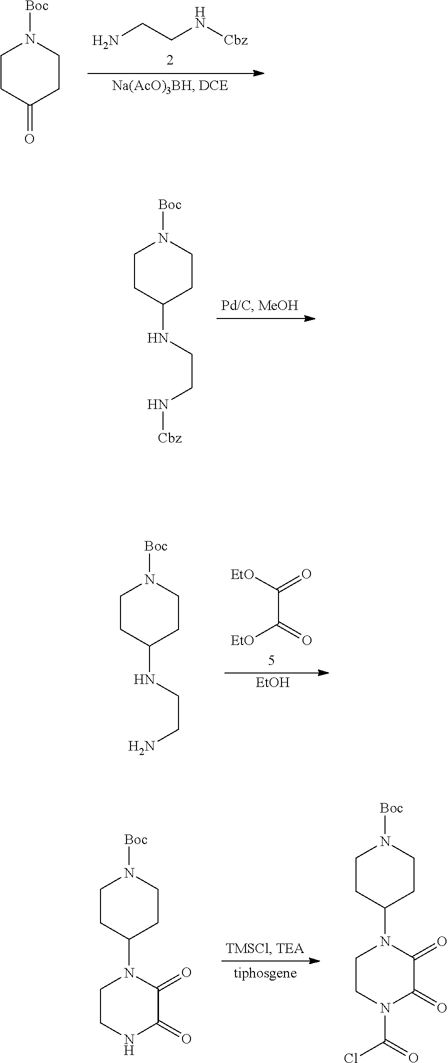

### Step 1a.

To a solution of tert-butyl 4-oxopiperidine-1-carboxylate (40 g, 200 mmol) and tert-butyl (2-aminoethyl)carbamate (38.8 g, 200 mmol) in DME (600 mL) at RT was added Na(OAc)₃BH (127.2 g, 600 mmol) in portions. The reaction mixture was stirred at RT overnight. It was washed by NaHCO₃ and brine. The organic phase was evaporated in vacuo to give crude. The crude product was purified by flash silica gel chromatography (0-10% MeOH/DCM) to give the desired intermediate (68.7 g, 90.9%).

### Step 1b.

To a solution of tert-butyl 4-((2-(((benzyloxy)carbonyl)amino)ethyl)amino)piperidine-1-carboxylate (68.7 g, 182 mmol) in MeOH (600 mL) at RT was added Pd/C. The reaction mixture was stirred at H₂ overnight. The heterogeneous mixture was filtered through Celite and concentrated in vacuo to provide a sticky brown oil (42 g, 94.8%).

### Step 1c.

The intermediate (42 g, 172.5 mmol) was dissolved in EtOH (1200 mL), diethyl oxalate (27.6 g, 189 mmol) was added. The mixture was refluxed for overnight. The reaction was concentrated. The product was purified by flash chromatography on silica gel (0-10% MeOH/DCM) to give the desired intermediate (40 g, 77.9%).

### Step 1d.

A solution of tert-butyl 4-(2,3-dioxopiperazin-1-yl)piperidine-1-carboxylate (40 g, 134.5 mmol) in DCM (250 mL)/THF (250 mL) was cooled to −40° C. TEA (20.2 mL, 161.4 mmol, 1.2 equiv.) was added. The mixture was warmed to 0° C. for 1 h. Triphosgene (16 g, 5.38 mmol, 0.4 equiv.) in THF (200 mL) was added and warmed to RT for 1 h. The heterogeneous mixture was filtered through Celite and concentrated in vacuo to provide a sticky brown oil. Et₂O (1000 mL) was added, and the product was triturated at RT overnight and filtered. The resulting solid was washed with Et₂O (200 mL) and dried in vacuo to provide the title compound (37.3 g, 77%).

### Step 2: Synthesis of tert-butyl 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

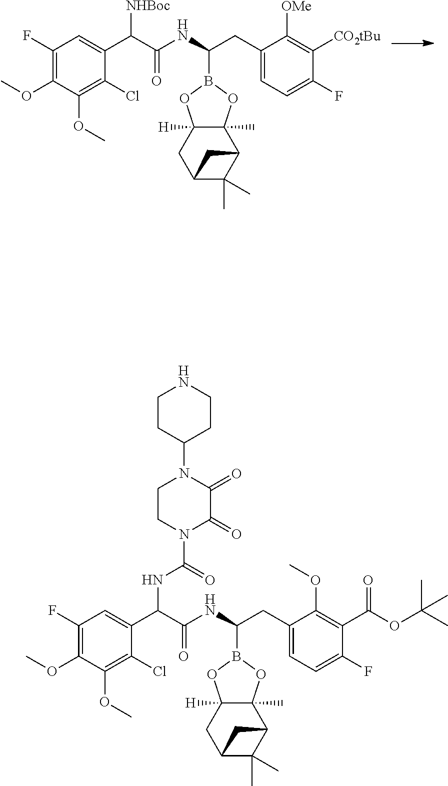 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(C3CCNCC3)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

The tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate was prepared in a similar manner to the synthesis of Example 400 (Steps 1 to 6).

To tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 20 g (25 mmol) at 0° C. was added 1 N hydrochloric acid in diethyl ether (400 mL) and warmed at RT for 18 h. The reaction was concentrated in vacuo to give tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride, 16 g, 91.4%. ESI-MS m/z 693 (M+H)⁺.

To tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 16 g (23 mmol) in dichloromethane (150 mL) at 0° C. was added N,N-diisopropylethylamine 12.7 mL (69 mmol, 3 eq), followed by tert-butyl 4-(4-(chlorocarbonyl)-2,3-dioxopiperazin-1-yl)piperidine-1-carboxylate (12.4 g, 34.5 mmol, 1.5 eq) and the reaction was warmed at RT for 2 h. The product was quenched with water, washed with brine, dried over sodium sulfate and concentrated to give tert-butyl 4-(4-((2-(((R)-2-(3-(tert-butoxycarbonyl)-4-fluoro-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(2-chloro-5-fluoro-3,4-dimethoxyphenyl)-2-oxoethyl)carbamoyl)-2,3-dioxopiperazin-1-yl)piperidine-1-carboxylate, 18.7 g, 80%. ESI-MS m/z 1016 (M+H)⁺.

The above crude product, 18.7 g (18.4 mmol) at 0° C. was added 1 N hydrochloric acid in diethyl ether (400 mL) and warmed at RT for 4 h. The reaction was concentrated in vacuo to tert-butyl 3-((2R)-2-(2-(2-chloro-5-fluoro-3,4-dimethoxyphenyl)-2-(2,3-dioxo-4-(piperidin-4-yl)piperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride, 16 g, ESI-MS m/z 916 (M+H)⁺.

### Step 3: Synthesis of (R)-3-((S)-2-(2-chloro-5-fluoro-3,4-dihydroxyphenyl)-2-(4-(1-((4-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(4-(1-((4-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

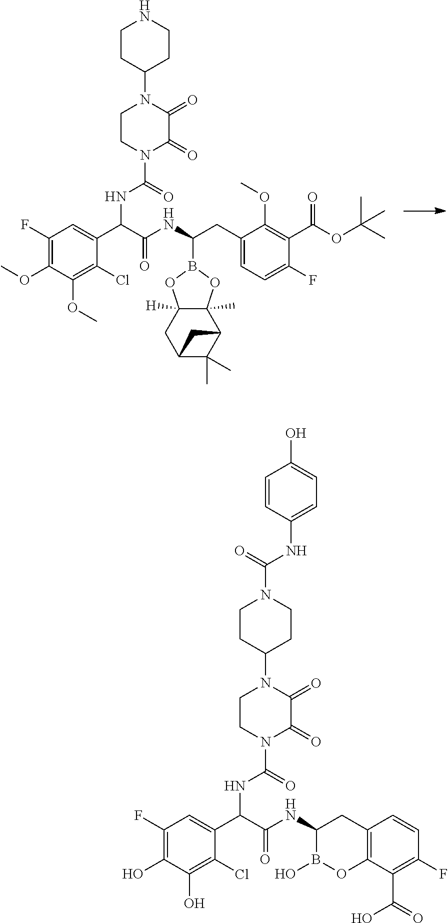 [SMILES: COc1c(F)cc(C(NC(=O)N2CCN(C3CCNCC3)C(=O)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(O)c1c(F)ccc2c1OB(O)[C@@H](NC(=O)C(NC(=O)N1CCN(C3CCN(C(=O)Nc4ccc(O)cc4)CC3)C(=O)C1=O)c1cc(F)c(O)c(O)c1Cl)C2]

i) To 4-methoxyaniline (0.16 g, 1.3 mmol) in THF (20 mL) was added triethylamine 0.56 mL (3.9 mmol, 3 eq), followed by triphosgene (0.154 g, 0.52 mmol, 0.4 eq) in THF (5 mL) was added and warmed to RT for 1 h. It was used in the next step without further purification.

To tert-butyl 3-((2R)-2-(2-(2-chloro-5-fluoro-3,4-dimethoxyphenyl)-2-(2,3-dioxo-4-(piperidin-4-yl)piperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (0.92 g, 1 mmol) in dichloromethane (8 mL) was added triethylamine (0.43 mL, 3 mmol, 3 eq), followed by above solution at 0° C. and the reaction mixture was stirred at RT for 18 h. The product was quenched with water, washed with brine, dried over sodium sulfate and concentrated to give the product tert-butyl 3-((2R)-2-(2-(2-chloro-5-fluoro-3,4-dimethoxyphenyl)-2-(4-(1-((4-methoxyphenyl)carbamoyl)piperidin-4-yl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 0.74 g, 70%. ESI-MS m/z 1065 (M+H)⁺.

ii) To the above crude product tert-butyl 3-((2R)-2-(2-(2-chloro-5-fluoro-3,4-dimethoxyphenyl)-2-(4-(1-((4-methoxyphenyl)carbamoyl)piperidin-4-yl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 0.74 g (0.7 mmol) in dichloromethane at −78° C. was added 1 N boron tribromide in dichloromethane (10.5 mL, 10.5 mmol, 15 eq) and warmed at RT for 18 h. The reaction was quenched with water, concentrated, and purified on the reverse phase HPLC (5-80% ACN—H₂O+0.1% trifluoroacetic acid) purification, the title compounds were collected as the first eluting and the second eluting peak ESI m/z 801 (M+H)⁺.

### Example 427: Synthesis of (3R)-3-(2-(2-chloro-5-fluoro-3,4-dihydroxyphenyl)-2-(4-(1-(4-hydroxybenzoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 4-methoxybenzoyl chloride in place of 3-methoxybenzoyl chloride in Step 3, the title compound was prepared after reversed phase HPLC purification. ESI-MS m/z 786/788 (M+H)⁺/(M+H+2)⁺.

### Example 428: Synthesis of (3R)-3-(2-(2-chloro-5-fluoro-3,4-dihydroxyphenyl)-2-(4-(1-((3-hydroxyphenyl)sulfonyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 3-methoxybenzenesulfonyl chloride in place of 3-methoxybenzoyl chloride in Step 3, the title compound was prepared after reversed phase HPLC purification. ESI-MS m/z 822/824 (M+H)⁺/(M+H+2)⁺.

### Example 429: Synthesis of (3R)-3-(2-(2-chloro-5-fluoro-3,4-dihydroxyphenyl)-2-(4-(1-((4-hydroxyphenyl)sulfonyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 4-methoxybenzenesulfonyl chloride in place of 3-methoxybenzoyl chloride in Step 3, the title compound was prepared after reversed phase HPLC purification. ESI-MS m/z 822/824 (M+H)⁺/(M+H+2)⁺.

### Example 430: Synthesis of (R)-3-((S)-2-(2-chloro-5-fluoro-3,4-dihydroxyphenyl)-2-(3-(1-(3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

### Step 1. Synthesis of tert-butyl 4-(3-(chlorocarbonyl)-2-oxoimidazolidin-1-yl)piperidine-1-carboxylate

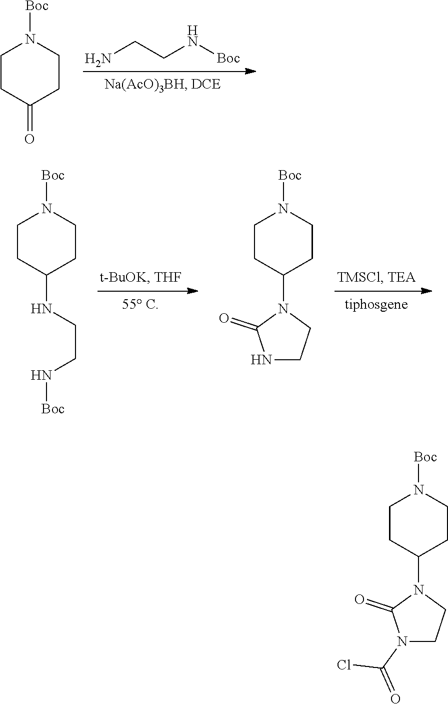

### Step 1a.

To a solution of tert-butyl 4-oxopiperidine-1-carboxylate (40 g, 200 mmol) and tert-butyl (2-aminoethyl)carbamate (32 g, 200 mmol) in DME (600 mL) at RT was added Na(OAc)₃BH (127.2 g, 600 mmol) in portions. The reaction mixture was stirred at RT overnight. It was washed by NaHCO₃ and brine. The organic phase was evaporated in vacuo to give crude product. The crude product was purified by flash silica gel chromatography (0-10% MeOH/DCM) to give the desired intermediate (61.8 g, 90%).

### Step 1b.

The above intermediate (61.8 g, 180 mmol) was dissolved in THF (1200 mL), t-BuOK (72.8 g, 650 was added. The mixture was stirred at 55° C. overnight. The reaction was diluted with ethyl acetate, washed with water then brine, dried over sodium sulfate, and concentrated. The product was purified by flash chromatography on silica gel (0-10% MeOH/DCM) to give the desired intermediate (45.7 g, 94%).

### Step 1c.

A solution of tert-butyl 4-(2-oxoimidazolidin-1-yl)piperidine-1-carboxylate (45.7 g, 169 mmol) in DCM (200 mL)/THF (200 mL) was cooled to −40° C. TEA (25.4 mL, 203 mmol, 1.2 equiv.) was added. The mixture was warmed to 0° C. for 1 h. Triphosgene (20 g, 67.6 mmol, 0.4 equiv.) in THF (200 mL) was added and warmed to RT for 1 h. The heterogeneous mixture was filtered through Celite and concentrated in vacuo to provide a sticky brown oil. Et₂O (1000 mL) was added, and the product was triturated at RT overnight and filtered. The resulting solid was washed with Et₂O (200 mL) and dried in vacuo to provide the title compound (44.8 g, 80%).

### Step 2: Synthesis of tert-butyl 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

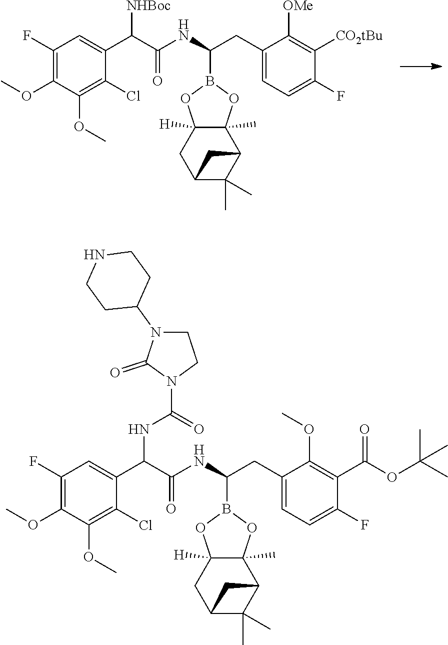 [SMILES: COc1c(F)cc(C(NC(=O)OC(C)(C)C)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; COc1c(F)cc(C(NC(=O)N2CCN(C3CCNCC3)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC]

The tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate was prepared in a similar manner to the synthesis of Example 400 (Steps 1 to 6).

To tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 20 g (25 mmol) at 0° C. was added 1 N hydrochloric acid in diethyl ether (400 mL) and warmed at RT for 18 h. The reaction was concentrated in vacuo to give tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride, 16 g, 91.4%. ESI-MS m/z 693 (M+H)⁺.

To tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride 16 g (23 mmol) in dichloromethane (150 mL) at 0° C. was added N,N-diisopropylethylamine 12.7 mL (69 mmol, 3 eq), followed by tert-butyl 4-(3-(chlorocarbonyl)-2-oxoimidazolidin-1-yl)piperidine-1-carboxylate (11.5 g, 34.5 mmol, 1.5 eq) and the reaction was warmed at RT for 2 h. The product was quenched with water, washed with brine, dried over sodium sulfate and concentrated to give tert-butyl 4-(3-((2-(((R)-2-(3-(tert-butoxycarbonyl)-4-fluoro-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(2-chloro-5-fluoro-3,4-dimethoxyphenyl)-2-oxoethyl)carbamoyl)-2-oxoimidazolidin-1-yl)piperidine-1-carboxylate, 20 g, 87.9%. ESI-MS m/z 988 (M+H)⁺.

The above crude product, 20 g (20 mmol) at 0° C. was added 1 N hydrochloric acid in diethyl ether (400 mL) and warmed at RT for 4 h. The reaction was concentrated in vacuo to tert-butyl 3-((2R)-2-(2-(4-(3-aminopropyl)-2,3-dioxopiperazine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride, 16 g, ESI-MS m/z 888 (M+H)⁺.

### Step 3: Synthesis of (R)-3-((S)-2-(2-chloro-5-fluoro-3,4-dihydroxyphenyl)-2-(3-(1-(3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

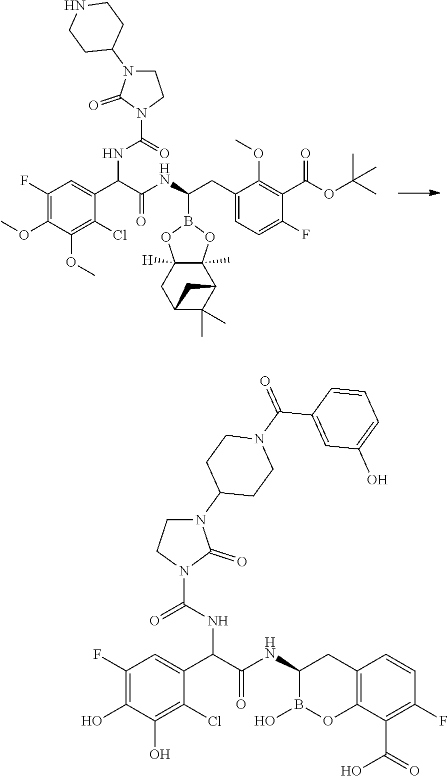 [SMILES: COc1c(F)cc(C(NC(=O)N2CCN(C3CCNCC3)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(O)c1c(F)ccc2c1OB(O)[C@@H](NC(=O)C(NC(=O)N1CCN(C3CCN(C(=O)c4cccc(O)c4)CC3)C1=O)c1cc(F)c(O)c(O)c1Cl)C2]

i) To the above crude product, tert-butyl 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoatehydrochloride (0.89 g, 1 mmol) in dichloromethane (8 mL) was added triethylamine (0.43 mL, 3 mmol, 3 eq), followed by 3-methoxybenzoyl chloride (0.187 g, 1.1 mmol, 1.1 eq) at 0° C. and the reaction mixture was stirred at RT for 18 h. The product was quenched with water, washed with brine, dried over sodium sulfate and concentrated to give the tert-butyl 3-((2R)-2-(2-(2-chloro-5-fluoro-3,4-dimethoxyphenyl)-2-(3-(1-(3-methoxybenzoyl)piperidin-4-yl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate, 0.92 g, 90%. ESI-MS m/z 1022 (M+H)⁺.

ii) To the above crude product tert-butyl 3-((2R)-2-(2-(2-chloro-5-fluoro-3,4-dimethoxyphenyl)-2-(3-(1-(3-methoxybenzoyl)piperidin-4-yl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (0.92 g, 0.9 mmol) in dichloromethane at −78° C. was added 1 N boron tribromide in dichloromethane (13.5 mL, 13.5 mmol, 15 eq) and warmed at RT for 18 h. The reaction was quenched with water, concentrated and purified on the reverse phase HPLC (5-80% ACN—H₂O+0.1% trifluoroacetic acid) purification, the title compounds were collected as the first eluting and the second eluting peak ESI m/z 758 (M+H)⁺.

### Example 431: Synthesis of (R)-3-((S)-2-(2-chloro-5-fluoro-3,4-dihydroxyphenyl)-2-(3-(1-(2-fluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(2-fluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2-fluoro-3-methoxybenzoyl chloride in place of 3-methoxybenzoyl chloride in Step 3. After reversed phase HPLC purification, the title compounds were collected as the first eluting and the second eluting peak. ESI m/z 776 (M+H)⁺.

### Example 432: Synthesis of (R)-3-((S)-2-(3-(1-(2-chloro-3-hydroxybenzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid and (R)-3-((R)-2-(3-(1-(2-chloro-3-hydroxybenzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2-chloro-3-methoxybenzoyl chloride in place of 3-methoxybenzoyl chloride in Step 3. After reversed phase HPLC purification, the title compounds were collected as the first eluting and the second eluting peak. ESI m/z 792 (M+H)⁺.

### Example 433: Synthesis of (R)-3-((S)-2-(2-chloro-5-fluoro-3,4-dihydroxyphenyl)-2-(3-(1-(2-fluoro-5-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(2-fluoro-5-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2-fluoro-5-methoxybenzoyl chloride in place of 3-methoxybenzoyl chloride in Step 3. After reversed phase HPLC purification, the title compounds were collected as the first eluting and the second eluting peak. ESI m/z 776 (M+H)⁺.

### Example 434: Synthesis of (R)-3-((S)-2-(2-chloro-5-fluoro-3,4-dihydroxyphenyl)-2-(3-(1-(2-chloro-5-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(2-chloro-5-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2-chloro-5-methoxybenzoyl chloride in place of 3-methoxybenzoyl chloride in Step 3. After reversed phase HPLC purification, the title compounds were collected as the first eluting and the second eluting peak. ESI m/z 792 (M+H)⁺.

### Example 435: Synthesis of (R)-3-((S)-2-(2-chloro-5-fluoro-3,4-dihydroxyphenyl)-2-(3-(1-(4-fluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(4-fluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 4-fluoro-3-methoxybenzoyl chloride in place of 3-methoxybenzoyl chloride in Step 3. After reversed phase HPLC purification, the title compounds were collected as the first eluting and the second eluting peak. ESI m/z 776 (M+H)⁺.

### Example 436: Synthesis of (R)-3-((S)-2-(2-chloro-5-fluoro-3,4-dihydroxyphenyl)-2-(3-(1-(3-fluoro-5-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(3-fluoro-5-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 3-fluoro-5-methoxybenzoyl chloride in place of 3-methoxybenzoyl chloride in Step 3. After reversed phase HPLC purification, the title compounds were collected as the first eluting and the second eluting peak. ESI m/z 776 (M+H)⁺.

### Example 437: Synthesis of (R)-3-((S)-2-(2-chloro-5-fluoro-3,4-dihydroxyphenyl)-2-(3-(1-(2,4-difluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(2,4-difluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2,4-difluoro-3-methoxybenzoyl chloride in place of 3-methoxybenzoyl chloride in Step 3. After reversed phase HPLC purification, the title compounds were collected as the first eluting and the second eluting peak. ESI m/z 794 (M+H)⁺.

### Example 438: Synthesis of (R)-3-((R)-2-(3-(1-(2-chloro-4-fluoro-3-hydroxybenzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2-chloro-4-fluoro-3-methoxybenzoyl chloride in place of 3-methoxybenzoyl chloride in Step 3. ESI m/z 810 (M+H)⁺.

### Example 439: Synthesis of (R)-3-((S)-2-(2-chloro-5-fluoro-3,4-dihydroxyphenyl)-2-(3-(1-(2,6-difluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(2,6-difluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2,6-difluoro-3-methoxybenzoyl chloride in place of 3-methoxybenzoyl chloride in Step 3. After reversed phase HPLC purification, the title compounds were collected as the first eluting and the second eluting peak. ESI m/z 794 (M+H)⁺.

### Example 440: Synthesis of (R)-3-((S)-2-(2-chloro-5-fluoro-3,4-dihydroxyphenyl)-2-(3-(1-(2-chloro-6-fluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(2-chloro-6-fluoro-3-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2-chloro-6-fluoro-3-methoxybenzoyl chloride in place of 3-methoxybenzoyl chloride in Step 3. After reversed phase HPLC purification, the title compounds were collected as the first eluting and the second eluting peak. ESI m/z 810 (M+H)⁺.

### Example 441: Synthesis of (R)-3-((S)-2-(2-chloro-5-fluoro-3,4-dihydroxyphenyl)-2-(2-oxo-3-(1-(2,4,5-trifluoro-3-hydroxybenzoyl)piperidin-4-yl)imidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(2-oxo-3-(1-(2,4,5-trifluoro-3-hydroxybenzoyl)piperidin-4-yl)imidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2,4,5-trifluoro-3-methoxybenzoyl chloride in place of 3-methoxybenzoyl chloride in Step 3. After reversed phase HPLC purification, the title compounds were collected as the first eluting and the second eluting peak. ESI m/z 812 (M+H)⁺.

### Example 442: Synthesis of (R)-3-((S)-2-(2-chloro-5-fluoro-3,4-dihydroxyphenyl)-2-(3-(1-(3,5-dihydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(3,5-dihydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 3,5-dimethoxybenzoyl chloride in place of 3-methoxybenzoyl chloride in Step 3. After reversed phase HPLC purification, the title compounds were collected as the first eluting and the second eluting peak. ESI m/z 774 (M+H)⁺.

### Example 443: Synthesis of (R)-3-((S)-2-(2-chloro-5-fluoro-3,4-dihydroxyphenyl)-2-(3-(1-(4-fluoro-3,5-dihydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(4-fluoro-3,5-dihydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 4-fluoro-3,5-dimethoxybenzoyl chloride in place of 3-methoxybenzoyl chloride in Step 3. After reversed phase HPLC purification, the title compounds were collected as the first eluting and the second eluting peak. ESI m/z 792 (M+H)⁺.

### Example 444: Synthesis of (R)-3-((S)-2-(2-chloro-5-fluoro-3,4-dihydroxyphenyl)-2-(3-(1-(2,4-difluoro-3,5-dihydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-(2,4-difluoro-3,5-dihydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 2,4-difluoro-3,5-dimethoxybenzoyl chloride in place of 3-methoxybenzoyl chloride in Step 3. After reversed phase HPLC purification, the title compounds were collected as the first eluting and the second eluting peak. ESI m/z 810 (M+H)⁺

### Example 445: Synthesis of (R)-3-((R)-2-(2-chloro-5-fluoro-3,4-dihydroxyphenyl)-2-(3-(1-((3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

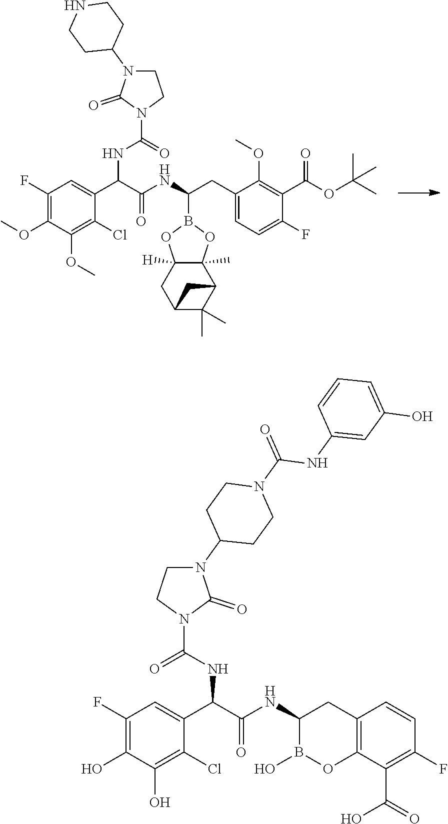 [SMILES: COc1c(F)cc(C(NC(=O)N2CCN(C3CCNCC3)C2=O)C(=O)N[C@@H](Cc2ccc(F)c(C(=O)OC(C)(C)C)c2OC)B2O[C@@H]3C[C@@H]4C[C@@H](C4(C)C)[C@]3(C)O2)c(Cl)c1OC ; O=C(O)c1c(F)ccc2c1OB(O)[C@@H](NC(=O)[C@H](NC(=O)N1CCN(C3CCN(C(=O)Nc4cccc(O)c4)CC3)C1=O)c1cc(F)c(O)c(O)c1Cl)C2]

i) To 3-methoxyaniline (0.16 g, 1.3 mmol) in THF (20 mL) was added triethylamine 0.56 mL (3.9 mmol, 3 eq), followed by triphosgene (0.154 g, 0.52 mmol, 0.4 equiv.) in THF (5 mL) was added and warmed to RT for 1 h. It was used in the next step without further purification.

To tert-butyl 3-((2R)-2-(2-(2-chloro-5-fluoro-3,4-dimethoxyphenyl)-2-(2,3-dioxo-4-(piperidin-4-yl)piperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (0.92 g, 1 mmol) in dichloromethane (8 mL) was added triethylamine (0.43 mL, 3 mmol, 3 eq), followed by above solution at 0° C. and the reaction mixture was stirred at RT for 18 h. The product was quenched with water, washed with brine, dried over sodium sulfate and concentrated to give the product tert-butyl 3-((2R)-2-(2-(2-chloro-5-fluoro-3,4-dimethoxyphenyl)-2-(4-(1-((3-methoxyphenyl)carbamoyl)piperidin-4-yl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 0.851 g, 80%. ESI-MS m/z 1065 (M+H)⁺.

ii) To the above crude tert-butyl 3-((2R)-2-(2-(2-chloro-5-fluoro-3,4-dimethoxyphenyl)-2-(4-(1-((3-methoxyphenyl)carbamoyl)piperidin-4-yl)-2,3-dioxopiperazine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 0.851 g (0.8 mmol) in dichloromethane at −78° C. was added 1 N boron tribromide in dichloromethane (12 mL, 12 mmol, 15 eq) and warmed at RT for 18 h. The reaction was quenched with water, concentrated and purified on the reverse phase HPLC (5-80% ACN—H₂O+0.1% trifluoroacetic acid) purification, the title compound was collected. ESI m/z 773 (M+H)⁺.

### Example 446: Synthesis of (R)-3-((S)-2-(2-chloro-5-fluoro-3,4-dihydroxyphenyl)-2-(3-(1-((2-fluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-((2-fluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 2-fluoro-3-methoxyaniline in place of 3-methoxyaniline. After reversed phase HPLC purification, the title compounds were collected as the first eluting and the second eluting peak. ESI m/z 791 (M+H)⁺.

### Example 447: Synthesis of (R)-3-((R)-2-(3-(1-((2-chloro-3-hydroxyphenyl)carbamoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 2-chloro-3-methoxyaniline in place of 3-methoxyaniline. ESI m/z 807 (M+H)⁺.

### Example 448: Synthesis of (R)-3-((S)-2-(2-chloro-5-fluoro-3,4-dihydroxyphenyl)-2-(3-(1-((2-chloro-5-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-((2-chloro-5-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 2-chloro-5-methoxyaniline in place of 3-methoxyaniline. After reversed phase HPLC purification, the title compounds were collected as the first eluting and the second eluting peak. ESI m/z 807 (M+H)⁺.

### Example 449: Synthesis of (R)-3-((S)-2-(2-chloro-5-fluoro-3,4-dihydroxyphenyl)-2-(3-(1-((4-fluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-((4-fluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 4-fluoro-3-methoxyaniline in place of 3-methoxyaniline. After reversed phase HPLC purification, the title compounds were collected as the first eluting and the second eluting peak. ESI m/z 791 (M+H)⁺.

### Example 450: Synthesis of (R)-3-((S)-2-(2-chloro-5-fluoro-3,4-dihydroxyphenyl)-2-(3-(1-((3-fluoro-5-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-((3-fluoro-5-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 3-fluoro-5-methoxyaniline in place of 3-methoxyaniline. After reversed phase HPLC purification, the title compounds were collected as the first eluting and the second eluting peak. ESI m/z 791 (M+H)⁺.

### Example 451: Synthesis of (R)-3-((S)-2-(2-chloro-5-fluoro-3,4-dihydroxyphenyl)-2-(3-(1-((2-fluoro-5-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-((2-fluoro-5-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 2-fluoro-5-methoxyaniline in place of 3-methoxyaniline. After reversed phase HPLC purification, the title compounds were collected as the first eluting and the second eluting peak. ESI m/z 791 (M+H)⁺.

### Example 452: Synthesis of (3R)-3-(2-(2-chloro-5-fluoro-3,4-dihydroxyphenyl)-2-(3-(1-((2,4-difluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 2,4-difluoro-3-methoxyaniline in place of 3-methoxyaniline. ESI m/z 809 (M+H)⁺.

### Example 454: Synthesis of (R)-3-((S)-2-(2-chloro-5-fluoro-3,4-dihydroxyphenyl)-2-(3-(1-((2,6-difluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-((2,6-difluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 2,6-difluoro-3-methoxyaniline in place 3-methoxyaniline. After reversed phase HPLC purification, the title compounds were collected as the first eluting and the second eluting peak. ESI m/z 809 (M+H)⁺.

### Example 455: Synthesis of (R)-3-((S)-2-(2-chloro-5-fluoro-3,4-dihydroxyphenyl)-2-(3-(1-((2-chloro-6-fluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(3-(1-((2-chloro-6-fluoro-3-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 2-chloro-6-fluoro-3-methoxyaniline in place of 3-methoxyaniline. After reversed phase HPLC purification, the title compounds were collected as the first eluting and the second eluting peak. ESI m/z 825 (M+H)⁺.

### Example 456: Synthesis of (R)-3-((R)-2-(2-chloro-5-fluoro-3,4-dihydroxyphenyl)-2-(3-(1-((3,5-dihydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 3,5-dimethoxyaniline in place of 3-methoxyaniline. ESI m/z 789 (M+H)⁺.

### Examples 459-A and 459-B: Synthesis of (R)-3-((S)-2-(2-chloro-5-fluoro-3,4-dihydroxyphenyl)-2-(2-oxo-3-(1-((2,4,5-trifluoro-3-hydroxyphenyl)carbamoyl)piperidin-4-yl)imidazolidine-1-carboxamido)acetamido)-7-fluoro-2-hydroxy-3,4-dihydro-2H-benzo[e][1,2]oxaborinine-8-carboxylic acid and (R)-3-((R)-2-(2-chloro-5-fluoro-3,4-dihydroxyphenyl)-2-(2-oxo-3-(1-((2,4,5-trifluoro-3-hydroxyphenyl)carbamoyl)piperidin-4-yl)imidazolidine-1-carboxamido)acetamido)-7-fluoro-2-hydroxy-3,4-dihydro-2H-benzo[e][1,2]oxaborinine-8-carboxylic acid

The title compounds were prepared in a similar manner to the synthesis of Example 445, utilizing 2,4,5-trifluoro-3-methoxyaniline in place of 3-methoxyaniline. After reversed phase HPLC purification, the title compounds were collected as the first eluting (A) and the second eluting (B) peak. ESI m/z 827 (M+H)⁺.

### Example 460: Synthesis of (R)-3-((R)-2-(2-chloro-5-fluoro-3,4-dihydroxyphenyl)-2-(3-(1-nicotinoylpiperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

### Step 1. Synthesis of 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoic acid

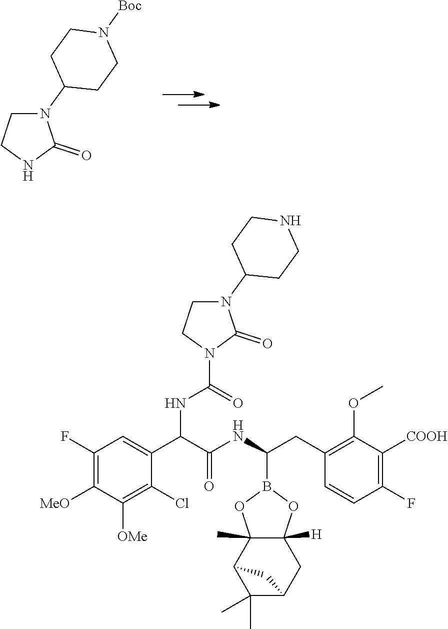 [SMILES: CC(C)(C)OC(=O)N1CCC(N2CCNC2=O)CC1 ; COc1c(F)cc(C(NC(=O)N2CCN(C3CCNCC3)C2=O)C(=O)N[C@@H](Cc2ccc(F)c(C(=O)O)c2OC)B2O[C@@H]3C[C@@H]4C[C@@H](C4(C)C)[C@]3(C)O2)c(Cl)c1OC]

In a similar manner to the synthesis of the intermediate of Step 2 of Example 293, utilizing tert-butyl 4-(2-oxoimidazolidin-1-yl)piperidine-1-carboxylate in place of tert-butyl 4-(2,3-dioxopiperazin-1-yl)piperidine-1-carboxylate in Step 1, the title compound was prepared as HCl salt which was used without further purification for the next step. ESI-MS m/z 832/834 (M+H)⁺/(M+H+2)⁺.

### Step 2. Synthesis of (R)-3-((R)-2-(2-chloro-5-fluoro-3,4-dihydroxyphenyl)-2-(3-(1-nicotinoylpiperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

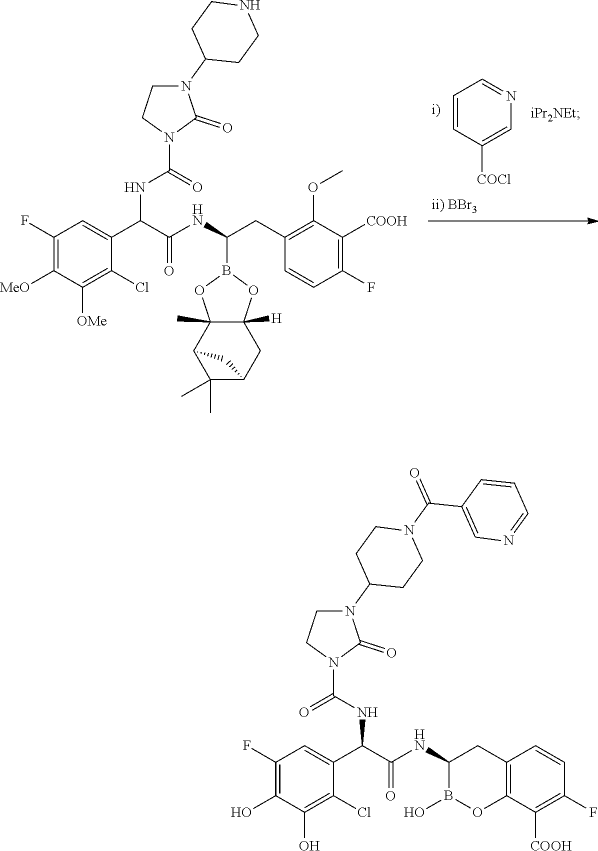 [SMILES: COc1c(F)cc(C(NC(=O)N2CCN(C3CCNCC3)C2=O)C(=O)N[C@@H](Cc2ccc(F)c(C(=O)O)c2OC)B2O[C@@H]3C[C@@H]4C[C@@H](C4(C)C)[C@]3(C)O2)c(Cl)c1OC ; O=C(Cl)c1cccnc1 ; O=C(O)c1c(F)ccc2c1OB(O)[C@@H](NC(=O)[C@H](NC(=O)N1CCN(C3CCN(C(=O)c4cccnc4)CC3)C1=O)c1cc(F)c(O)c(O)c1Cl)C2]

Following the same reaction conditions described in Step 3 of Example 293, utilizing nicotinoyl chloride in place of 3-methoxybenzoyl chloride, the above intermediate was converted to the title compound which was purified by reversed phase HPLC. ESI-MS m/z 743/745 (M+H)⁺/(M+H+2)⁺.

### Example 461: Synthesis of (R)-3-((R)-2-(2-chloro-5-fluoro-3,4-dihydroxyphenyl)-2-(3-(1-isonicotinoylpiperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 460, utilizing isonicotinoyl chloride in place of nicotinoyl chloride in Step 2, the title compound was prepared after reversed phase HPLC purification. ESI-MS m/z 822/824 (M+H)⁺/(M+H+2)⁺.

### Example 462: Synthesis of (R)-3-((S)-2-(2-chloro-5-fluoro-3,4-dihydroxyphenyl)-2-(3-(1-(4-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 430, utilizing 4-methoxybenzoyl chloride in place of 3-methoxybenzoyl chloride in Step 3. After reversed phase HPLC purification, the title compound was collected as the first eluting peak. ESI m/z 758 (M+H)⁺.

### Example 463: Synthesis of (R)-3-((R)-2-(2-chloro-5-fluoro-3,4-dihydroxyphenyl)-2-(3-(1-(4-hydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 462. After reversed phase HPLC purification, the title compound was collected as the second eluting peak. ESI m/z 758 (M+H)⁺.

### Example 464: Synthesis of (3R)-3-(2-(2-chloro-5-fluoro-3,4-dihydroxyphenyl)-2-(3-(1-(5-hydroxynicotinoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 460, utilizing 5-(benzyloxy)nicotinoyl chloride (which was made from 5-(benzyloxy)nicotinic acid by treatment with excess of oxalyl chloride in DCM at RT) in place of nicotinoyl chloride in Step 2, the title compound was prepared after reversed phase HPLC purification. ESI-MS m/z 759/761 (M+H)⁺/(M+H+2)⁺.

### Example 465: Synthesis of (3R)-3-(2-(2-chloro-5-fluoro-3,4-dihydroxyphenyl)-2-(2-oxo-3-(1-(2-oxo-1,2-dihydropyridine-4-carbonyl)piperidin-4-yl)imidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 460, utilizing 2-(benzyloxy)isonicotinoyl chloride (which was made from 2-(benzyloxy)isonicotinic acid by treatment with excess of oxalyl chloride in DCM at RT) in place of nicotinoyl chloride in Step 2, the title compound was prepared after reversed phase HPLC purification. ESI-MS m/z 759/761 (M+H)⁺/(M+H+2)⁺.

### Example 466: Synthesis of (3R)-3-(2-(2-chloro-5-fluoro-3,4-dihydroxyphenyl)-2-(4-(3-(2-fluoroisonicotinamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-fluoroisonicotinoyl chloride in place of 3-fluoroisonicotinoyl chloride in Step 8. ESI-MS m/z 763 (M+H)⁺.

### Example 467: Synthesis of (R)-3-((S)-2-(2-chloro-5-fluoro-3,4-dihydroxyphenyl)-2-(3-(1-((4-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 445, utilizing 4-methoxyaniline in place of 3-methoxyaniline. After reversed phase HPLC purification, the title compound was collected as the first eluting peak. ESI m/z 773 (M+H)⁺.

### Example 468: Synthesis of (R)-3-((R)-2-(2-chloro-5-fluoro-3,4-dihydroxyphenyl)-2-(3-(1-((4-hydroxyphenyl)carbamoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 467. After reversed phase HPLC purification, the title compound was collected as the second eluting peak. ESI m/z 773 (M+H)⁺.

### Example 469: Synthesis of (3R)-3-(2-(3-(1-(1H-benzo[d][1,2,3]triazole-5-carbonyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 460, utilizing 1H-benzo[d][1,2,3]triazole-5-carbonyl chloride in place of nicotinoyl chloride in Step 2, the title compound was prepared after reversed phase HPLC purification. ESI-MS m/z 783/785 (M+H)⁺/(M+H+2)⁺.

### Example 470: Synthesis of (R)-3-((S)-2-(4-(3-(2-chloro-4-hydroxybenzamido)propyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2-chloro-4-methoxybenzoyl chloride in place of 3-fluoroisonicotinoyl chloride in Step 8. After reversed phase HPLC purification, the title compound was collected as the first eluting peak. ESI-MS m/z 794 (M+H)⁺.

### Example 471: Synthesis of (R)-3-((R)-2-(4-(3-(2-chloro-4-hydroxybenzamido)propyl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 470. After reversed phase HPLC purification, the title compound was collected as the second eluting peak. ESI-MS m/z 794 (M+H)⁺.

### Example 472: Synthesis of (R)-3-((S)-2-(2-chloro-5-fluoro-3,4-dihydroxyphenyl)-2-(4-(3-(2,5-difluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 400, utilizing 2,5-difluoro-4-methoxybenzoyl chloride in place of 3-fluoroisonicotinoyl chloride in Step 8. After reversed phase HPLC purification, the title compound was collected as the first eluting peak. ESI-MS m/z 796 (M+H)⁺.

### Example 473: Synthesis of (R)-3-((R)-2-(2-chloro-5-fluoro-3,4-dihydroxyphenyl)-2-(4-(3-(2,5-difluoro-4-hydroxybenzamido)propyl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 472. After reversed phase HPLC purification, the title compound was collected as the second eluting peak. ESI-MS m/z 796 (M+H)⁺.

### Example 474: Synthesis of (3R)-3-(2-(2-chloro-5-fluoro-3,4-dihydroxyphenyl)-2-(4-(1-(2-(3-hydroxyphenyl)acetyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 2-(3-methoxyphenyl)acetyl chloride in place of 3-methoxybenzoyl chloride in Step 3, the title compound was prepared after reversed phase HPLC purification. ESI-MS m/z 800/802 (M+H)⁺/(M+H+2)⁺.

### Example 475: Synthesis of (3R)-3-(2-(2-chloro-5-fluoro-3,4-dihydroxyphenyl)-2-(4-((1-(3-hydroxybenzoyl)piperidin-4-yl)methyl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing tert-butyl 4-((2,3-dioxopiperazin-1-yl)methyl)piperidine-1-carboxylate in place of tert-butyl 4-(2,3-dioxopiperazin-1-yl)piperidine-1-carboxylate in Step 1, the title compound was prepared after reversed phase HPLC purification. ESI-MS m/z 800/802 (M+H)⁺/(M+H+2)⁺.

### Example 476: (3R)-3-(2-(2-chloro-5-fluoro-3,4-dihydroxyphenyl)-2-(4-(1-(2,4-dihydroxybenzoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing 2,4-dimethoxybenzoyl chloride in place of 3-methoxybenzoyl chloride in Step 3, the title compound was prepared after reversed phase HPLC purification. ESI-MS m/z 802/804 (M+H)⁺/(MH+2)⁺.

### Example 477: (3R)-3-(2-(2-chloro-5-fluoro-3,4-dihydroxyphenyl)-2-(4-((1-((3-hydroxyphenyl)carbamoyl)piperidin-4-yl)methyl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 424, utilizing tert-butyl 4-((2,3-dioxopiperazin-1-yl)methyl)piperidine-1-carboxylate in place of tert-butyl 4-(2,3-dioxopiperazin-1-yl)piperidine-1-carboxylate in Step 1, the title compound was prepared after reversed phase HPLC purification. ESI-MS m/z 815/817 (M+H)⁺/(MH+2)⁺.

### Example 478: Synthesis of (3R)-3-(2-(2-chloro-5-fluoro-3,4-dihydroxyphenyl)-2-(4-(1-((2-fluoro-3-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 2-fluoro-3-methoxyaniline in place of 4-methoxyaniline in Step 3. ESI m/z 819 (M+H)⁺.

### Example 479: Synthesis of (3R)-3-(2-(2-chloro-5-fluoro-3,4-dihydroxyphenyl)-2-(4-(1-((2-fluoro-5-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 2-fluoro-5-methoxyaniline in place of 4-methoxyaniline in Step 3. ESI m/z 819 (M+H)⁺.

### Example 480: Synthesis of (3R)-3-(2-(2-chloro-5-fluoro-3,4-dihydroxyphenyl)-2-(4-(1-((4-fluoro-3-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 4-fluoro-3-methoxyaniline in place of 4-methoxyaniline in Step 3. ESI m/z 819 (M+H)⁺.

### Example 481: Synthesis of (3R)-3-(2-(2-chloro-5-fluoro-3,4-dihydroxyphenyl)-2-(4-(1-((3-fluoro-5-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 3-fluoro-5-methoxyaniline in place of 4-methoxyaniline in Step 3. ESI m/z 819 (M+H)⁺.

### Example 482: Synthesis of (R)-3-((R)-2-(4-(1-((2-chloro-3-hydroxyphenyl)carbamoyl)piperidin-4-yl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 2-chloro-3-methoxyaniline in place of 4-methoxyaniline in Step 3. ESI m/z 835 (M+H)⁺.

### Example 483: Synthesis of (3R)-3-(2-(2-chloro-5-fluoro-3,4-dihydroxyphenyl)-2-(4-(1-((2-chloro-5-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 2-chloro-5-methoxyaniline in place of 4-methoxyaniline in Step 3. ESI m/z 835 (M+H)⁺.

### Example 484: Synthesis of (3R)-3-(2-(2-chloro-5-fluoro-3,4-dihydroxyphenyl)-2-(4-(1-((3-chloro-5-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 3-chloro-5-methoxyaniline in place of 4-methoxyaniline in Step 3. ESI m/z 835 (M+H)⁺.

### Example 485: Synthesis of (3R)-3-(2-(2-chloro-5-fluoro-3,4-dihydroxyphenyl)-2-(4-(1-((3-hydroxybenzyl)sulfonyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

In a similar manner to the synthesis of Example 293, utilizing (3-methoxyphenyl)methanesulfonyl chloride in place of 3-methoxybenzoyl chloride in Step 3, the title compound was prepared after reversed phase HPLC purification. ESI-MS m/z 836/838 (M+H)⁺/(M+H+2)⁺.

### Example 486: Synthesis of (3R)-3-(2-(2-chloro-5-fluoro-3,4-dihydroxyphenyl)-2-(4-(1-((2,4-difluoro-3-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 2,4-difluoro-3-methoxyaniline in place of 4-methoxyaniline in Step 3. ESI m/z 837 (M+H)⁺.

### Example 487: Synthesis of (3R)-3-(2-(2-chloro-5-fluoro-3,4-dihydroxyphenyl)-2-(4-(1-((2,6-difluoro-3-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 2,6-difluoro-3-methoxyaniline in place of 4-methoxyaniline in Step 3. ESI m/z 837 (M+H)⁺.

### Example 488: Synthesis of (3R)-3-(2-(2-chloro-5-fluoro-3,4-dihydroxyphenyl)-2-(4-(1-((3,4-difluoro-5-hydroxyphenyl)carbamoyl)piperidin-4-yl)-2,3-dioxopiperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 3,4-difluoro-5-methoxyaniline in place of 4-methoxyaniline in Step 3. ESI m/z 837 (M+H)⁺.

### Example 489: Synthesis of (3R)-3-(2-(4-(1-((4-chloro-2-fluoro-3-hydroxyphenyl)carbamoyl)piperidin-4-yl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 4-chloro-2-fluoro-3-methoxyaniline in place of 4-methoxyaniline in Step 3. ESI m/z 853 (M+H)⁺.

### Example 490: Synthesis of (3R)-3-(2-(4-(1-((2-chloro-4-fluoro-5-hydroxyphenyl)carbamoyl)piperidin-4-yl)-2,3-dioxopiperazine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 2-chloro-4-fluoro-5-methoxyaniline in place of 4-methoxyaniline in Step 3. ESI m/z 853 (M+H)⁺.

### Example 491: Synthesis of (R)-3-((R)-2-(2-chloro-5-fluoro-3,4-dihydroxyphenyl)-2-(2,3-dioxo-4-(1-((2,4,5-trifluoro-3-hydroxyphenyl)carbamoyl)piperidin-4-yl)piperazine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 426, utilizing 2,4,5-trifluoro-3-methoxyaniline in place of 4-methoxyaniline in Step 3. ESI m/z 855 (M+H)⁺.

### Example 492: Synthesis of (R)-3-((R)-2-(2-chloro-5-fluoro-3,4-dihydroxyphenyl)-2-(2-oxo-3-(1-(pyridin-3-ylmethyl)piperidin-4-yl)imidazolidine-1-carboxamido)acetamido)-7-fluoro-2-hydroxy-3,4-dihydro-2H-benzo[e][1,2]oxaborinine-8-carboxylic acid

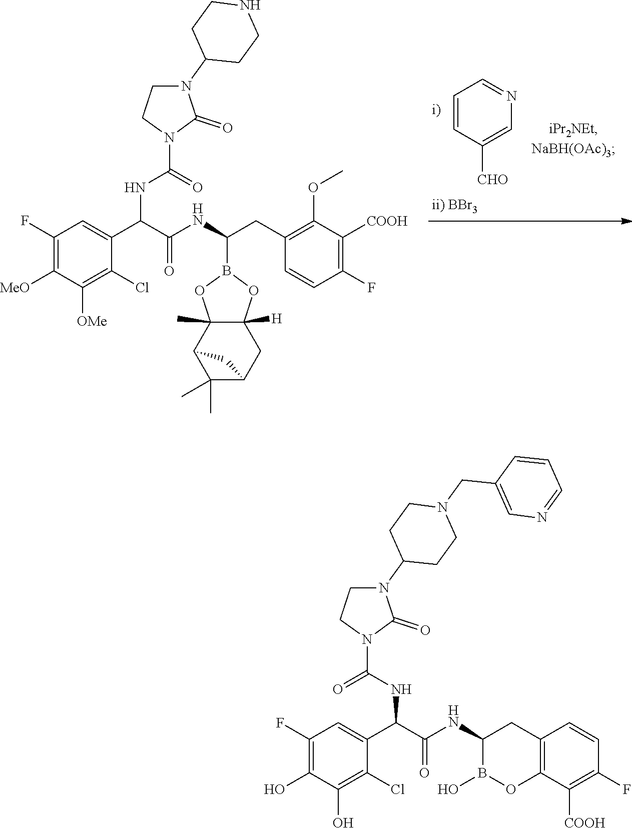 [SMILES: COc1c(F)cc(C(NC(=O)N2CCN(C3CCNCC3)C2=O)C(=O)N[C@@H](Cc2ccc(F)c(C(=O)O)c2OC)B2O[C@@H]3C[C@@H]4C[C@@H](C4(C)C)[C@]3(C)O2)c(Cl)c1OC ; O=Cc1cccnc1 ; O=C(O)c1c(F)ccc2c1OB(O)[C@@H](NC(=O)[C@H](NC(=O)N1CCN(C3CCN(Cc4cccnc4)CC3)C1=O)c1cc(F)c(O)c(O)c1Cl)C2]

Following the same reaction conditions described in Step 15 and Step 16 of Example 200, utilizing 3-pyridinecarbxaldehyde in place of thiazole-2-carboxaldehyde, the intermediate from Step 1 of Example 460 was converted to the title compound which was purified by reversed phase HPLC. ESI-MS m/z 729/731 (M+H)⁺/(M+H+2)⁺.

### Example 493: Synthesis of (R)-3-((R)-2-(2-chloro-5-fluoro-3,4-dihydroxyphenyl)-2-(2-oxo-3-(1-(pyridin-4-ylmethyl)piperidin-4-yl)imidazolidine-1-carboxamido)acetamido)-7-fluoro-2-hydroxy-3,4-dihydro-2H-benzo[e][1,2]oxaborinine-8-carboxylic acid

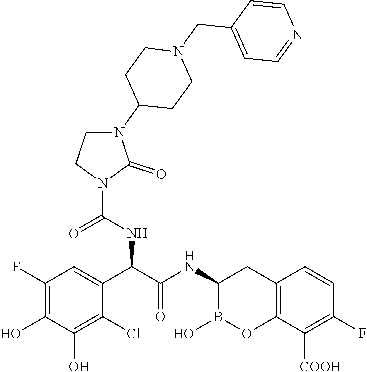 [SMILES: O=C(O)c1c(F)ccc2c1OB(O)[C@@H](NC(=O)[C@H](NC(=O)N1CCN(C3CCN(Cc4ccncc4)CC3)C1=O)c1cc(F)c(O)c(O)c1Cl)C2]

In a similar manner to the synthesis of Example 492, utilizing 4-pyridinecarbxaldehyde in place of 3-pyridinecarbxaldehyde, the title compound was prepared after reversed phase HPLC purification. ESI-MS m/z 729/731 (M+H)⁺/(M+H+2)⁺.

### Example 494: Synthesis of (R)-3-((R)-2-(3-(1-(2-chloro-3,4-dihydroxybenzoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 2-(4-(bis(benzyloxy)phosphoryl)phenyl)-2-((tert-butyloxycarbonyl)amino)acetic acid

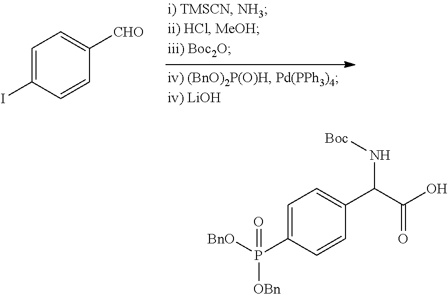 [SMILES: O=Cc1ccc(I)cc1 ; CC(C)(C)OC(=O)NC(C(=O)O)c1ccc(P(=O)(OCc2ccccc2)OCc2ccccc2)cc1]

### Step 1a.

To 4-iodobenzaldehyde (46.2 g, 200 mmol) at 0° C. was added 7 N ammonia in methanol (600 mL), followed by trimethylsilyl cyanide (29.8 g, 300 mmol, 1.5 eq). The mixture was stirred at 45° C. for 24 h and concentrated in vacuo.

### Step 1b.

The crude product was dissolved in 3 N hydrochloric acid in methanol (400 mL), stirred at 50° C. for 36 h and concentrated in vacuo.

### Step 1c.

To the crude product in DCM (400 mL) at 0° C. was added triethylamine (30.3 g, 300 mmol, 3 eq) followed by di-tert-butyl dicarbonate (65.4 g, 300 mmol, 1.5 eq). The reaction was warmed to RT for 12 h and concentrated in vacuo. The crude product was purified by flash silica gel chromatography (10% ethyl acetate/hexanes) to give the desired intermediate (51 g, 66% for 3 steps).

### Step 1d.

To methyl 2-((tert-butyloxycarbonyl)amino)-2-(4-iodophenyl)acetate (20 g, 52 mmol) was added N,N-diisopropylethylamine 20.2 g, 156 mmol, 3 eq), Pd(PPh₃)₄ (11.8 g, 10.2 mmol, 20 mol %), dibenzyl phosphite (26.7 g, 102 mmol, 2 eq), followed by anhydrous toluene (400 mL) and the mixture was stirred at RT for 5-7 h under argon. The reaction was diluted with ethyl acetate, washed with water then brine, dried over sodium sulfate, and concentrated. The product was purified by flash chromatography on silica gel (35-50% ethyl acetate/hexanes) to give the desired intermediate (27 g, 99%).

### Step 1e.

To methyl 2-(4-(bis(benzyloxy)phosphoryl)phenyl)-2-((tert-butoxycarbonyl)amino)acetate (27 g, 51.4 mmol) in THF (100 mL)/H₂O (100 mL) was added lithium hydroxide monohydrate (6.48 g, 154 mmol, 3 eq) and the mixture was stirred at RT for 1 h. 2N Hydrochloric acid was added drop wise to obtain pH 2 and extracted with dichloromethane. The organic layer was washed with water, dried over sodium sulfate, and concentrated to give 20 g crude product. The product was purified by flash chromatography on silica gel (10-20% MeOH/DCM) to give the title compound (15.8 g, 60%) as a white solid. ESI-MS m/z 512 (M+H)⁺.

### Step 2. Synthesis of tert-butyl 4-(3-(chlorocarbonyl)-2-oxoimidazolidin-1-yl)piperidine-1-carboxylate

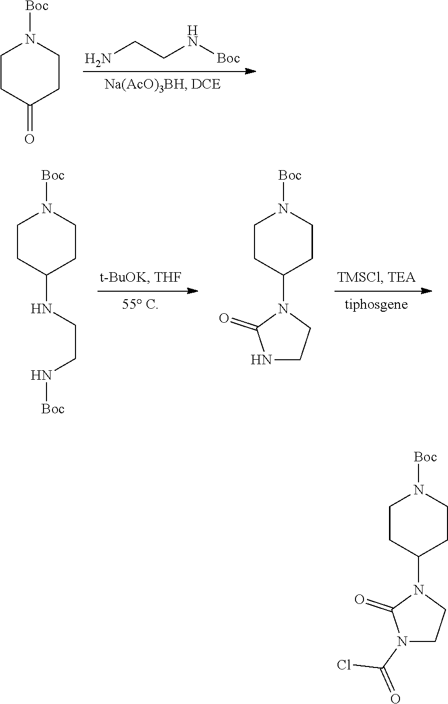

### Step 2a.

To a solution of tert-butyl 4-oxopiperidine-1-carboxylate (40 g, 200 mmol) and tert-butyl (2-aminoethyl)carbamate (32 g, 200 mmol) in DME (600 mL) at RT was added Na(OAc)₃BH (127.2 g, 600 mmol) in portions. The reaction mixture was stirred at RT overnight. It was washed by NaHCO₃ and brine. The organic phase was evaporated in vacuo to give crude product. The crude product was purified by flash silica gel chromatography (0-10% MeOH/DCM) to give the desired intermediate (61.8 g, 90%).

### Step 2b.

The above intermediate (61.8 g, 180 mmol) was dissolved in THF (1200 mL), t-BuOK (72.8 g, 650 was added. The mixture was stirred at 55° C. overnight. The reaction was diluted with ethyl acetate, washed with water then brine, dried over sodium sulfate, and concentrated. The product was purified by flash chromatography on silica gel (0-10% MeOH/DCM) to give the desired intermediate (45.7 g, 94%).

### Step 2c.

A solution of tert-butyl 4-(2-oxoimidazolidin-1-yl)piperidine-1-carboxylate (45.7 g, 169 mmol) in DCM (200 mL)/THF (200 mL) was cooled to −40° C. TEA (25.4 mL, 203 mmol, 1.2 equiv.) was added. The mixture was warmed to 0° C. for 1 h. Triphosgene (20 g, 67.6 mmol, 0.4 equiv.) in THF (200 mL) was added and warmed to RT for 1 h. The heterogeneous mixture was filtered through Celite and concentrated in vacuo to provide a sticky brown oil. Et₂O (1000 mL) was added, and the product was triturated at RT overnight and filtered. The resulting solid was washed with Et₂O (200 mL) and dried in vacuo to provide the title compound (44.8 g, 80%).

### Step 3. Synthesis of 2-(4-(bis(benzyloxy)phosphoryl)phenyl)-2-(3-(1-(2-chloro-3,4-dimethoxybenzoyl)piperidin-4-yl)-2-oxoimidazolidine-1-carboxamido)acetic acid

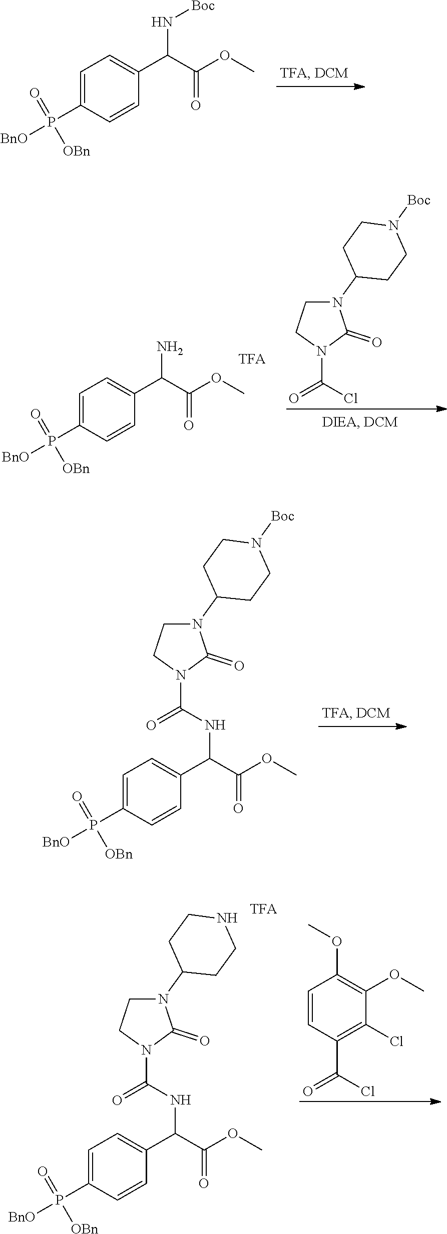 [SMILES: COC(=O)C(NC(=O)OC(C)(C)C)c1ccc(P(=O)(OCc2ccccc2)OCc2ccccc2)cc1 ; COC(=O)C(N)c1ccc(P(=O)(OCc2ccccc2)OCc2ccccc2)cc1 ; CC(C)(C)OC(=O)N1CCC(N2CCN(C(=O)Cl)C2=O)CC1 ; COC(=O)C(NC(=O)N1CCN(C2CCN(C(=O)OC(C)(C)C)CC2)C1=O)c1ccc(P(=O)(OCc2ccccc2)OCc2ccccc2)cc1 ; COC(=O)C(NC(=O)N1CCN(C2CCNCC2)C1=O)c1ccc(P(=O)(OCc2ccccc2)OCc2ccccc2)cc1 ; COc1ccc(C(=O)Cl)c(Cl)c1OC]

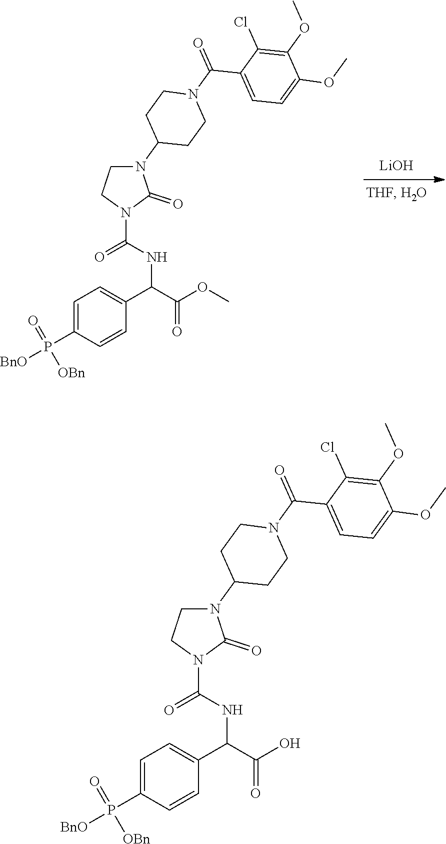 [SMILES: COC(=O)C(NC(=O)N1CCN(C2CCN(C(=O)c3ccc(OC)c(OC)c3Cl)CC2)C1=O)c1ccc(P(=O)(OCc2ccccc2)OCc2ccccc2)cc1 ; "LiOH" ; COc1ccc(C(=O)N2CCC(N3CCN(C(=O)NC(C(=O)O)c4ccc(P(=O)(OCc5ccccc5)OCc5ccccc5)cc4)C3=O)CC2)c(Cl)c1OC]

### Step 3a.

To a solution of 2-(4-(bis(benzyloxy)phosphoryl)phenyl)-2-((tert-butoxycarbonyl)amino)acetic acid (2.1 g, 4 mmol) in DCM (16.0 mL) at 0° C. was added TFA (4.0 mL). The solution was warmed to RT for 2 h then concentrated.

### Step 3b.

The crude product was dissolved in THF (40.0 mL)/NaHCO₃ (sat. aq., 40.0 mL) followed by addition of tert-butyl 4-(3-(chlorocarbonyl)-2-oxoimidazolidin-1-yl)piperidine-1-carboxylate (1.46 g, 4.4 mmol, 1.1 equiv.). The reaction was stirred at RT for 2 h. The reaction was diluted with H₂O (50 mL) and acidified until pH 2 with HCl (2 M). Ethyl acetate (200 mL) was added and the organic layer was washed with H₂O (2×50 mL) and brine (1×50 mL), dried (Na₂SO₄), and concentrated. The crude product was purified by silica gel chromatography (0-20% MeOH/DCM) to yield title compound (1.4 g, 56% over 2 steps). ESI-MS m/z 721 (M+H)⁺.

### Step 3c.

To a solution of tert-butyl 4-(3-((1-(4-(bis(benzyloxy)phosphoryl)phenyl)-2-methoxy-2-oxoethyl)carbamoyl)-2-oxoimidazolidin-1-yl)piperidine-1-carboxylate (1.4 g, 1.94 mmol) in DCM (16.0 mL) at 0° C. was added TFA (4.0 mL). The solution was warmed to RT for 2 h then concentrated.

### Step 3d.

The crude product was dissolved in THF (40.0 mL)/NaHCO₃ (sat. aq., 40.0 mL) followed by addition of 2-chloro-3,4-dimethoxybenzoyl chloride (0.5 g, 2.1 mmol, 1.1 equiv.). The reaction was stirred at RT for 2 h. The reaction was diluted with H₂O (50 mL) and acidified until pH 2 with HCl (2 M). Ethyl acetate (200 mL) was added and the organic layer was washed with H₂O (2×50 mL) and brine (1×50 mL), dried (Na₂SO₄), and concentrated. The crude product was purified by silica gel chromatography (0-20% MeOH/DCM) to yield title compound (660 mg, 45% over 2 steps). ESI-MS m/z 820 (M+H)⁺.

### Step 3e.

To methyl 2-(4-(bis(benzyloxy)phosphoryl)phenyl)-2-(3-(1-(2-chloro-3,4-dimethoxybenzoyl)piperidin-4-yl)-2-oxoimidazolidine-1-carboxamido)acetate (660 mg, 0.8 mmol) in THF (10 mL)/H₂O (10 mL) was added lithium hydroxide monohydrate (23 mg, 0.96 mmol, 1.2 eq) and the mixture was stirred at RT for 1 h. 2N Hydrochloric acid was added drop wise to obtain pH 2 and extracted with dichloromethane. The organic layer was washed with water, dried over sodium sulfate, and concentrated to give 20 g crude product. The product was purified by flash chromatography on silica gel (10-20% MeOH/DCM) to give the title compound (400 mg, 61%) as a white solid. ESI-MS m/z 806 (M+H)⁺.

### Step 4. Synthesis of (R)-3-((R)-2-(3-(1-(2-chloro-3,4-dihydroxybenzoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

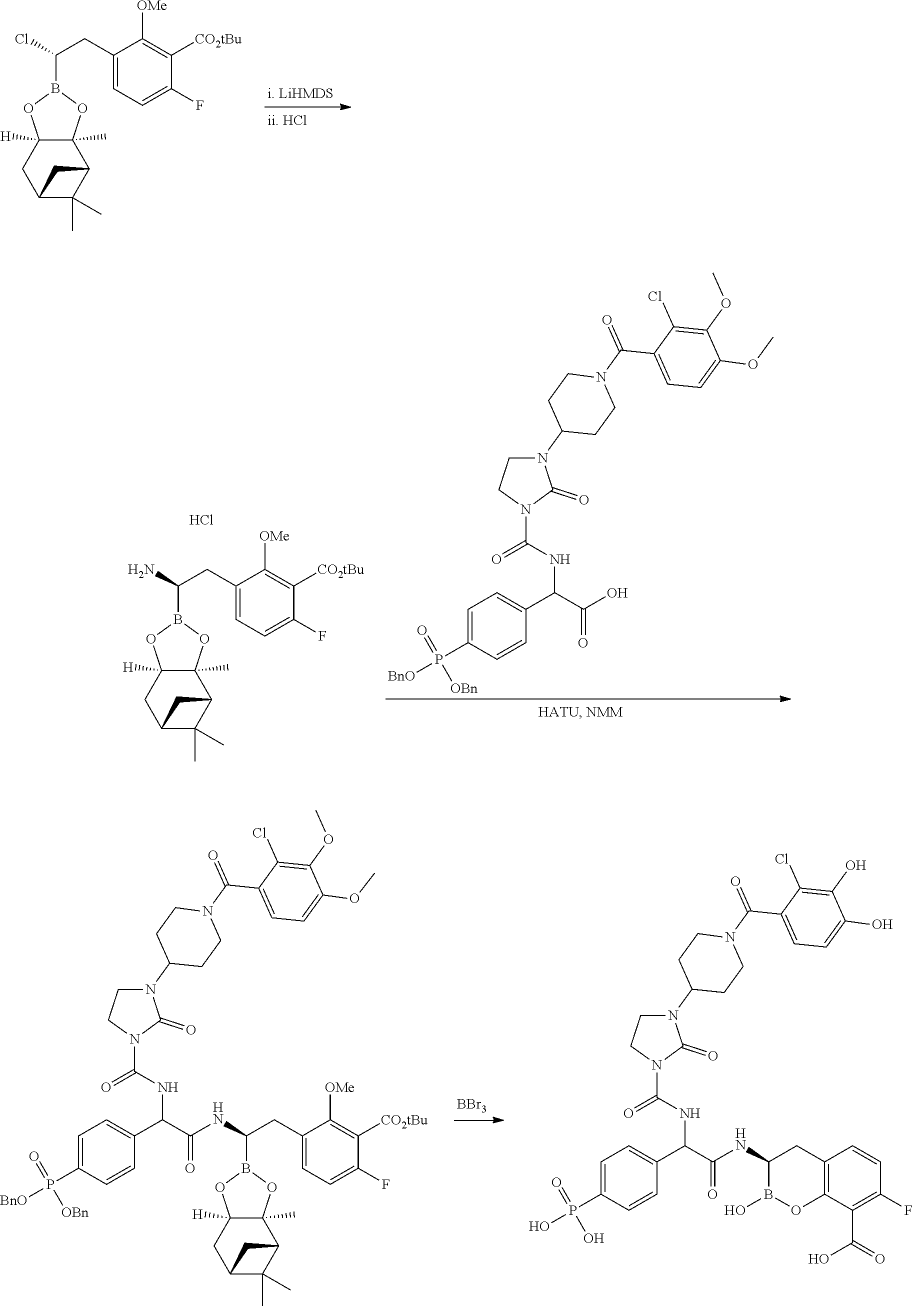

### Step 4a.

A solution of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (5.2 g, 11 mmol) (Example 200, Step 7) in THF (45 mL) was cooled to −78° C. LiHMDS (1 M in THF, 11 mL, 11 mmol, 1.0 equiv.) was added dropwise and the mixture was warmed to RT for 1 h. The solution was cooled to 0° C. and HCl (4 M in dioxane, 11 mL, 44 mmol, 4.0 eq) was added dropwise. The reaction was warmed to RT for 1 h then concentrated. Hexanes (200 mL) was added and stirred at RT overnight. The solid was filtered, washed with hexanes (2×100 mL), and dried to yield desired intermediate tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (5.8 g, 99%).

### Step 4b.

To a solution of tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (198 mg, 0.340 mmol), (3R)-3-(2-(3-(1-(2-chloro-3,4-dihydroxybenzoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid (400 mg, 0.50 mmol, 1.5 equiv.), and HATU (226 mg, 0.595 mmol, 1.75 equiv.) in DMA (3.4 mL) was added NMM (0.11 mL, 1.02 mmol, 3.0 equiv.). The reaction was stirred at RT for 30 min. and diluted with EtOAc (30 mL). The mixture was washed with H₂O (2×10 mL) and brine (xx mL), dried (Na₂SO₄), filtered, and concentrated. The crude product was purified by silica gel chromatography (0-5% MeOH/DCM) to yield desired tert-butyl 3-((2R)-2-(2-(4-(bis(benzyloxy)phosphoryl)phenyl)-2-(3-(1-(2-chloro-3,4-dimethoxybenzoyl)piperidin-4-yl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (330 mg, 53%) ESI m/z 1234 (M+H)⁺.

### Step 4c.

To a solution of tert-butyl 3-((2R)-2-(2-(4-(bis(benzyloxy)phosphoryl)phenyl)-2-(3-(1-(2-chloro-3,4-dimethoxybenzoyl)piperidin-4-yl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (330 mg, 0.26 mmol) in DCM (2.2 mL) at −78° C. was added BBr₃ (1 M in DCM, 2.2 mL, 2.2 mmol, 10 equiv.). The reaction was warmed to RT overnight then concentrated. The resulting crude product was purified by reverse-phase HPLC to yield (3R)-3-(2-(3-(1-(2-chloro-3,4-dihydroxybenzoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid (33.7 mg). ESI m/z 804 (M+H)⁺.

### Example 495: Synthesis of (R)-3-((R)-2-(3-(1-(2-chloro-5-fluoro-3,4-dihydroxybenzoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 494, utilizing 2-chloro-5-fluoro-3,4-dimethoxybenzoyl chloride in place of 2-chloro-3,4-dimethoxybenzoyl chloride in Step 3. After reversed phase HPLC purification in Step 4, the title compound was collected as the second eluting peak. ESI-MS m/z 822 (M+H)⁺.

### Example 496: Synthesis of (R)-3-((R)-2-(3-(1-(3-chloro-4,5-dihydroxybenzoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 494, utilizing 3-chloro-4,5-dimethoxybenzoyl chloride in place of 2-chloro-3,4-dimethoxybenzoyl chloride in Step 3. After reversed phase HPLC purification in Step 4, the title compound was collected ESI-MS m/z 804 (M+H)⁺.

### Example 497: Synthesis of (R)-3-((R)-2-(3-(1-((2-chloro-3,4-dihydroxyphenyl)carbamoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 494, utilizing 2-chloro-1-isocyanato-3,4-dimethoxybenzene in place of 2-chloro-3,4-dimethoxybenzoyl chloride in Step 3. After reversed phase HPLC purification in Step 4, the title compound was collected as the second eluting peak. ESI-MS m/z 819 (M+H)⁺.

### Example 498: Synthesis of (3R)-2-hydroxy-3-(2-(4-phosphonophenyl)-2-(pyrazolo[1,5-a]pyridine-6-carboxamido)acetamido)-3,4-dihydro-2H-benzo[e][1,2]oxaborinine-8-carboxylic acid

### Step 1. Synthesis of 2-(4-(bis(benzyloxy)phosphoryl)phenyl)-2-((tert-butoxycarbonyl)amino)acetic acid

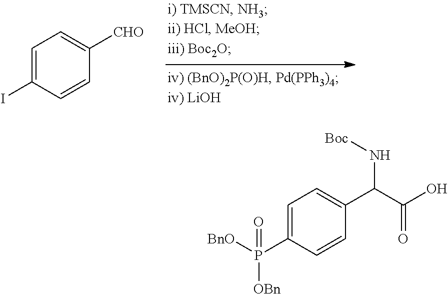 [SMILES: O=Cc1ccc(I)cc1 ; CC(C)(C)OC(=O)NC(C(=O)O)c1ccc(P(=O)(OCc2ccccc2)OCc2ccccc2)cc1]

### Step 1a.

To 4-iodobenzaldehyde (46.2 g, 200 mmol) at 0° C. was added 7 N ammonia in methanol (600 mL), followed by trimethylsilyl cyanide (29.8 g, 300 mmol, 1.5 eq). The mixture was stirred at 45° C. for 24 h and concentrated in vacuo.

### Step 1b.

The crude product was dissolved in 3 N hydrochloric acid in methanol (400 mL), stirred at 50° C. for 36 h and concentrated in vacuo.

### Step 1c.

To the crude product in DCM (400 mL) at 0° C. was added triethylamine (30.3 g, 300 mmol, 3 eq) followed by di-tert-butyl dicarbonate (65.4 g, 300 mmol, 1.5 eq). The reaction was warmed to RT for 12 h and concentrated in vacuo. The crude product was purified by flash silica gel chromatography (10% ethyl acetate/hexanes) to give the desired intermediate (51 g, 66% for 3 steps).

### Step 1d.

To methyl 2-((tert-butoxycarbonyl)amino)-2-(4-iodophenyl)acetate (20 g, 52 mmol) was added N,N-diisopropylethylamine 20.2 g, 156 mmol, 3 eq), Pd(PPh₃)₄ (11.8 g, 10.2 mmol, 20 mol %), dibenzyl phosphite (26.7 g, 102 mmol, 2 eq), followed by anhydrous toluene (400 mL) and the mixture was stirred at RT for 5-7 h under argon. The reaction was diluted with ethyl acetate, washed with water then brine, dried over sodium sulfate, and concentrated. The product was purified by flash chromatography on silica gel (35-50% ethyl acetate/hexanes) to give the desired intermediate (27 g, 99%).

### Step 1e.

To methyl 2-(4-(bis(benzyloxy)phosphoryl)phenyl)-2-((tert-butoxycarbonyl)amino)acetate (27 g, 51.4 mmol) in THF (100 mL)/H₂O (100 mL) was added lithium hydroxide monohydrate (6.48 g, 154 mmol, 3 eq) and the mixture was stirred at RT for 1 h. 2N Hydrochloric acid was added drop wise to obtain pH 2 and extracted with dichloromethane. The organic layer was washed with water, dried over sodium sulfate, and concentrated to give 20 g crude product. The product was purified by flash chromatography on silica gel (10-20% MeOH/DCM) to give the title compound (15.8 g, 60%) as a white solid. ESI-MS m/z 512 (M+H)⁺.

### Step 2. Synthesis of (3R)-3-(2-amino-2-(4-phosphonophenyl)acetamido)-2-hydroxy-3,4-dihydro-2H-benzo[e][1,2]oxaborinine-8-carboxylic acid

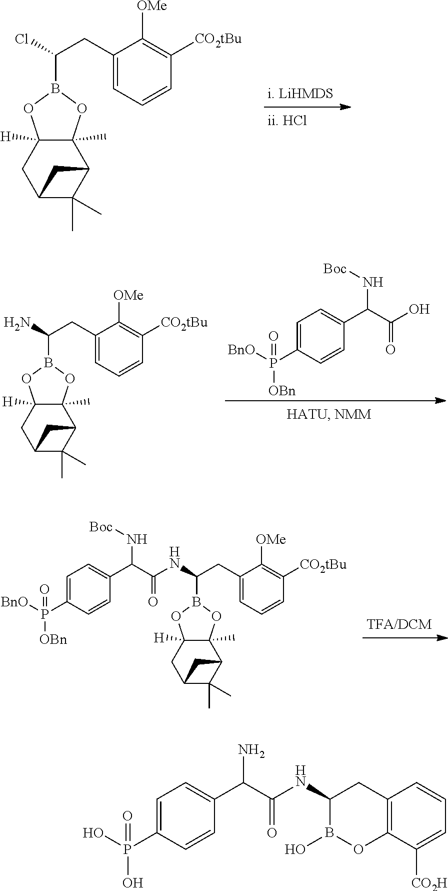 [SMILES: COc1c(C[C@@H](Cl)B2O[C@@H]3C[C@@H]4C[C@@H](C4(C)C)[C@]3(C)O2)cccc1C(=O)OC(C)(C)C ; COc1c(C[C@H](N)B2O[C@@H]3C[C@@H]4C[C@@H](C4(C)C)[C@]3(C)O2)cccc1C(=O)OC(C)(C)C ; CC(C)(C)OC(=O)NC(C(=O)O)c1ccc(P(=O)(OCc2ccccc2)OCc2ccccc2)cc1 ; COc1c(C[C@H](NC(=O)C(NC(=O)OC(C)(C)C)c2ccc(P(=O)(OCc3ccccc3)OCc3ccccc3)cc2)B2O[C@@H]3C[C@@H]4C[C@@H](C4(C)C)[C@]3(C)O2)cccc1C(=O)OC(C)(C)C ; NC(C(=O)N[C@H]1Cc2cccc(C(=O)O)c2OB1O)c1ccc(P(=O)(O)O)cc1]

### Step 2a.

A solution of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (4.9 g, 11 mmol) (Example 200, Step 7) in THF (45 mL) was cooled to −78° C. LiHMDS (1 M in THF, 11 mL, 11 mmol, 1.0 equiv.) was added dropwise and the mixture was warmed to RT for 1 h. The solution was cooled to 0° C. and HCl (4 M in dioxane, 11 mL, 44 mmol, 4.0 eq) was added dropwise. The reaction was warmed to RT for 1 h then concentrated. Hexanes (200 mL) was added and stirred at RT overnight. The solid was filtered, washed with hexanes (2×100 mL), and dried to yield desired intermediate tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-2-methoxybenzoate hydrochloride (5.5 g, 99%).

### Step 2b.

To a solution of tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-2-methoxybenzoate hydrochloride (1.7 g, 3.40 mmol), 2-(4-(bis(benzyloxy)phosphoryl)phenyl)-2-((tert-butoxycarbonyl)amino)acetic acid (3 g, 5.09 mmol, 1.5 equiv.), and HATU (2.26 g, 5.95 mmol, 1.75 equiv.) in DMA (34 mL) was added NMM (1.1 mL, 10.2 mmol, 3.0 equiv.). The reaction was stirred at RT for 30 min. and diluted with EtOAc (300 mL). The mixture was washed with H₂O (2×100 mL) and brine (300 mL), dried (Na₂SO₄), filtered, and concentrated. The crude product was purified by silica gel chromatography (0-5% MeOH/DCM) to yield desired tert-butyl 3-((2R)-2-(2-(4-(bis(benzyloxy)phosphoryl)phenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate (2 g, 64%) ESI m/z 923 (M+H)⁺.

### Step 2c.

To a solution of tert-butyl 3-((2R)-2-(2-(4-(bis(benzyloxy)phosphoryl)phenyl)-2-((tert-butoxycarbonyl)amino)acetamido)-2-((3aS,4S,6S,7aR)-3a,5,5-trimethylhexahydro-4,6-methanobenzo[d][1,3,2]dioxaborol-2-yl)ethyl)-2-methoxybenzoate (2 g, 2.16 mmol) in DCM (16.0 mL) at 0° C. was added TFA (4.0 mL). The solution was warmed to RT for 2 h then concentrated.

### Step 3. Synthesis of (3R)-2-hydroxy-3-(2-(4-phosphonophenyl)-2-(pyrazolo[1,5-a]pyridine-6-carboxamido)acetamido)-3,4-dihydro-2H-benzo[e][1,2]oxaborinine-8-carboxylic acid

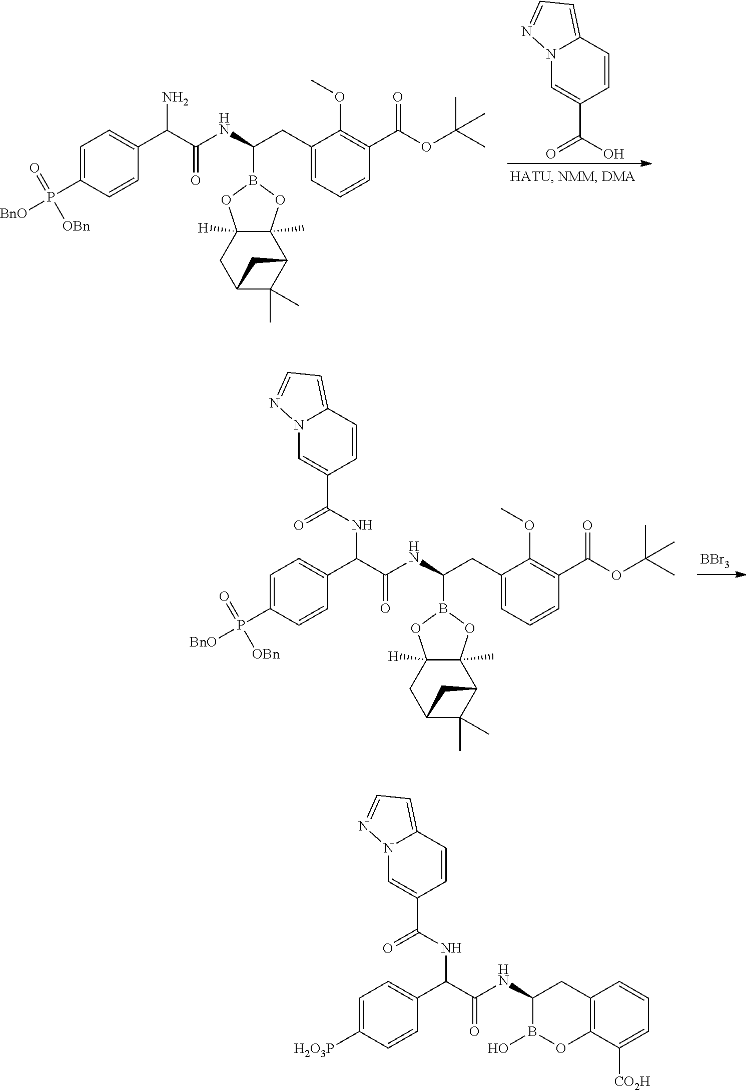

### Step 3a.

To a solution of tert-butyl 3-((2R)-2-(2-amino-2-(4-(bis(benzyloxy)phosphoryl)phenyl)acetamido)-2-((3aS,4S,6S,7aR)-3a,5,5-trimethylhexahydro-4,6-methanobenzo[d][1,3,2]dioxaborol-2-yl)ethyl)-2-methoxybenzoate (340 mg, 0.34 mmol), pyrazolo[1,5-a]pyridine-6-carboxylic acid (824 mg, 0.509 mmol, 1.5 equiv.), and HATU (226 mg, 0.595 mmol, 1.75 equiv.) in DMA (3.4 mL) was added NMM (1.1 mL, 1.02 mmol, 3.0 equiv.). The reaction was stirred at RT for 30 min. and diluted with EtOAc (30 mL). The mixture was washed with H₂O (2×10 mL) and brine (30 mL), dried (Na₂SO₄), filtered, and concentrated. The crude product was purified by silica gel chromatography (0-5% MeOH/DCM) to yield desired tert-butyl 3-((2R)-2-(2-(4-(bis(benzyloxy)phosphoryl)phenyl)-2-(pyrazolo[1,5-a]pyridine-6-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate. (197 mg, 60%) ESI m/z 967 (M+H)⁺.

### Step 3b.

To a solution of tert-butyl 3-((2R)-2-(2-(4-(bis(benzyloxy)phosphoryl)phenyl)-2-(pyrazolo[1,5-a]pyridine-6-carboxamido)acetamido)-2-((3aS,4S,6S,7aR)-3a,5,5-trimethylhexahydro-4,6-methanobenzo[d][1,3,2]dioxaborol-2-yl)ethyl)-2-methoxybenzoate (197 mg, 0.2 mmol) in DCM (2 mL) at −78° C. was added BBr₃ (1 M in DCM, 2 mL, 2.2 mmol, 10 equiv.). The reaction was warmed to RT overnight then concentrated. The resulting crude product was purified by reverse-phase HPLC to yield (3R)-2-hydroxy-3-(2-(4-phosphonophenyl)-2-(pyrazolo[1,5-a]pyridine-6-carboxamido)acetamido)-3,4-dihydro-2H-benzo[e][1,2]oxaborinine-8-carboxylic acid (14.9 mg). ESI m/z 565 (M+H)⁺.

### Example 499: Synthesis of (R)-2-hydroxy-3-((R)-2-(4-phosphonophenyl)-2-(pyrrolo[1,2-a]pyrimidine-3-carboxamido)acetamido)-3,4-dihydro-2H-benzo[e][1,2]oxaborinine-8-carboxylic acid

The title compound was prepared in a similar manner to the synthesis of Example 498, utilizing pyrrolo[1,2-a]pyrimidine-3-carboxylic acid in place of pyrazolo[1,5-a]pyridine-6-carboxylic acid in Step 3. After reversed phase HPLC purification in Step 4, the title compound was collected as the second eluting peak. ESI-MS m/z 565 (M+H)⁺.

### Example 500: Synthesis of (R)-3-((R)-2-(3-(1-((3-chloro-4,5-dihydroxyphenyl)carbamoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 494, utilizing 2-chloro-5-fluoro-1-isocyanato-3,4-dimethoxybenzene in place of 2-chloro-3,4-dimethoxybenzoyl chloride in Step 3. After reversed phase HPLC purification in Step 4, the title compound was collected as the second eluting peak. ESI-MS m/z 837 (M+H)⁺.

### Example 501: Synthesis of (R)-3-((R)-2-(3-(1-((3-chloro-4,5-dihydroxyphenyl)carbamoyl)piperidin-4-yl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 494, utilizing 3-chloro-1-isocyanato-4,5-dimethoxybenzene in place of 2-chloro-3,4-dimethoxybenzoyl chloride in Step 3. After reversed phase HPLC purification in Step 4, the title compound was collected as the second eluting peak. ESI-MS m/z 819 (M+H)⁺.

### Example 502: Synthesis of (R)-3-((R)-2-(4-(2-chloro-3,4-dihydroxybenzyl)-2,3-dioxopiperazine-1-carboxamido)-2-(3,5-difluoro-4-phosphonophenyl)acetamido)-7-fluoro-2-hydroxy-3,4-dihydro-2H-benzo[e][1,2]oxaborinine-8-carboxylic acid

The title compound was synthesized in a similar manner as Example 8, utilizing 3,5-difluoro-4-iodobenzaldehyde in place of 4-iodobenzaldehyde (Example 8, Step 1). ESI m/z 770 (M+H)⁺.

### Example 503: Synthesis of (R)-3-((R)-2-(4-(2-(2-chloro-3,4-dihydroxybenzamido)ethyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 2-(4-(bis(benzyloxy)phosphoryl)phenyl)-2-(4-(2-(2-chloro-3,4-dimethoxybenzamido)ethyl)-2,3-dioxopiperazine-1-carboxamido)acetic acid

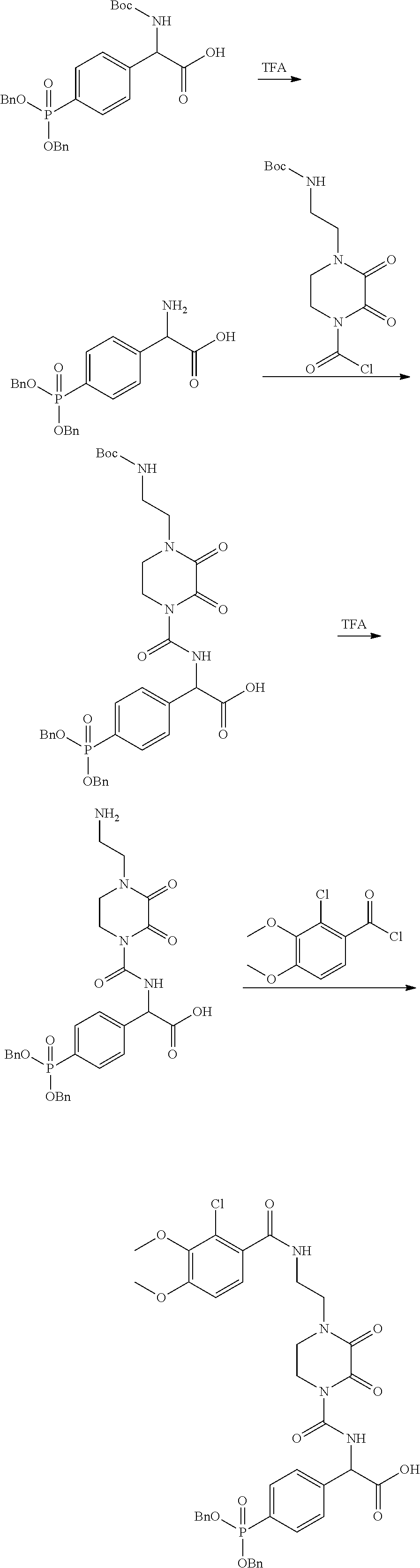

### Step 1a.

This reaction was performed in a similar manner to Example 8, Step 3a.

### Step 1b.

This reaction was performed in a similar manner to Example 8, Step 3b utilizing tert-butyl (2-(4-(chlorocarbonyl)-2,3-dioxopiperazin-1-yl)ethyl)carbamate in place of 4-(2-chloro-3,4-dimethoxybenzyl)-2,3-dioxopiperazine-1-carbonyl chloride. The product was purified by silica gel chromatography (0-5% MeOH/DCM) to yield desired intermediate (2.73 g, 79% over 2 steps).

### Step 1c.

To a solution of 2-(4-(bis(benzyloxy)phosphoryl)phenyl)-2-(4-(2-((tert-butoxycarbonyl)amino)ethyl)-2,3-dioxopiperazine-1-carboxamido)acetic acid (1.14 g, 1.64 mmol) in DCM (6.6 mL) at 0° C. was added TFA (1.64 mL). The solution was warmed to RT for 1 h then concentrated.

### Step 1d.

The crude product was dissolved in DCM (16 mL). Triethylamine (1.1 mL, 8.2 mmol, 5.0 equiv.) was added followed by 2-chloro-3,4-dimethoxybenzoyl chloride (424 mg, 1.80 mmol, 1.1 equiv.). The mixture was stirred for 1 h then quenched with NaHSO₄ (1.0 M, 10.0 mL). The layers were separated, and the aqueous layer was extracted with DCM (3×20 mL). The combined organic layers were dried (Na₂SO₄), filtered, and concentrated. The crude product was purified by silica gel chromatography (0-10% MeOH/DCM) to yield title compound (1.01 g, 78% over 2 steps).

### Step 2. Synthesis of (3R)-3-(2-(4-(2-(2-chloro-3,4-dihydroxybenzamido)ethyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 8, Step 4 utilizing (3R)-3-(2-(4-(2-(2-chloro-3,4-dihydroxybenzamido)ethyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid in place of 2-(4-(bis(benzyloxy)phosphoryl)phenyl)-2-(4-(2-chloro-3,4-dimethoxybenzyl)-2,3-dioxopiperazine-1-carboxamido)acetic acid. The desired compound was isolated by reverse-phase HPLC. ESI-MS m/z 792 (M+H)⁺.

### Example 504: Synthesis of (R)-3-((R)-2-(4-(2-chloro-5-fluoro-3,4-dihydroxybenzyl)-2,3-dioxopiperazine-1-carboxamido)-2-(4-phosphonophenyl)acetamido)-2-hydroxy-3,4-dihydro-2H-benzo[e][1,2]oxaborinine-8-carboxylic acid

### Step 1. Synthesis of 2-chloro-5-fluoro-3,4-dimethoxybenzaldehyde

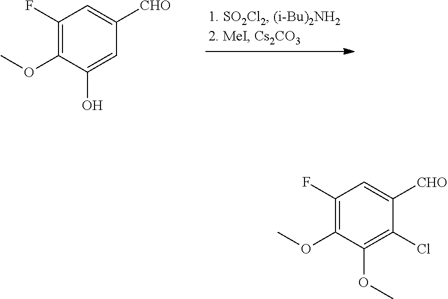

### Step 1a.

A solution of 3-fluoro-5-hydroxy-4-methoxybenzaldehyde (8.00 g, 47.0 mmol) in toluene (170 mL) was cooled to 0° C. (i-Bu)₂NH (4.00 mL, 49.4 mmol, 1.05 equiv.) was added followed by SO₂Cl₂ (0.82 mL, 4.70 mmol, 0.1 equiv.) slowly. The mixture was heated to 70° C. overnight then cooled to RT and quenched with H₂O (50 mL). The layers were separated, and the aq. layer was extracted with EtOAc (3×50 mL). The combined organic layers were dried (Na₂SO₄), filtered, and concentrated.

### Step 1b.

The crude product was dissolved in DMF (120 mL) and Cs₂CO₃ (38.3 g, 117 mmol, 2.5 equiv.) was added followed by MeI (8.8 mL, 140 mmol, 3.0 equiv.). The mixture was stirred at RT for 1 h, diluted with EtOAc (300 mL), and quenched with H₂O (150 mL). The layers were separated and the aq. layer was extracted with EtOAc (3×150 mL). The combined organic layers were washed with H₂O (3×75 mL) and brine (75 mL), dried (Na₂SO₄), filtered, and concentrated. The crude product was purified by silica gel chromatography (0-20%, EtOAc/Hexanes). ESI m/z 219 (M+H)⁺.

### Step 2. Synthesis of (3R)-3-(2-(4-(2-chloro-5-fluoro-3,4-dihydroxybenzyl)-2,3-dioxopiperazine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was synthesized in a similar manner as Example 8, utilizing 2-chloro-5-fluoro-3,4-dimethoxybenzaldehyde in place of 2-chloro-3,4-dimethoxybenzaldehyde (Example 8, Step 2), utilizing tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (Example 8, Step 4), ESI m/z 735 (M+H)⁺.

### Example 505: Synthesis of (3R)-3-(2-(3-((2-chloro-3,4-dihydroxyphenyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

### Step 1. Synthesis of 2-(4-(bis(benzyloxy)phosphoryl)phenyl)-2-((tert-butoxycarbonyl)amino)acetic acid

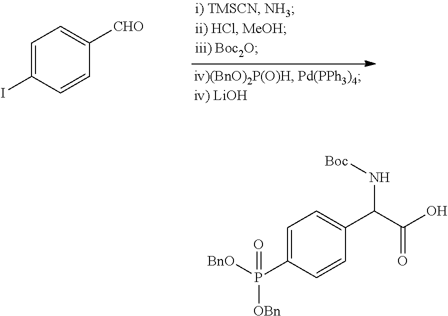 [SMILES: O=Cc1ccc(I)cc1 ; CC(C)(C)OC(=O)NC(C(=O)O)c1ccc(P(=O)(OCc2ccccc2)OCc2ccccc2)cc1]

### Step 1a.

To 4-iodobenzaldehyde (46.2 g, 200 mmol) at 0° C. was added 7 N ammonia in methanol (600 mL), followed by trimethylsilyl cyanide (29.8 g, 300 mmol, 1.5 eq). The mixture was stirred at 45° C. for 24 h and concentrated in vacuo.

### Step 1b.

The crude product was dissolved in 3 N hydrochloric acid in methanol (400 mL), stirred at 50° C. for 36 h and concentrated in vacuo.

### Step 1c.

To the crude product in DCM (400 mL) at 0° C. was added triethylamine (30.3 g, 300 mmol, 3 eq) followed by di-tert-butyl dicarbonate (65.4 g, 300 mmol, 1.5 eq). The reaction was warmed to RT for 12 h and concentrated in vacuo. The crude product was purified by flash silica gel chromatography (10% ethyl acetate/hexanes) to give the desired intermediate (51 g, 66% for 3 steps).

### Step 1d.

To methyl 2-((tert-butyloxycarbonyl)amino)-2-(4-iodophenyl)acetate (20 g, 52 mmol) was added N,N-diisopropylethylamine 20.2 g, 156 mmol, 3 eq), Pd(PPh₃)₄ (11.8 g, 10.2 mmol, 20 mol %), dibenzyl phosphite (26.7 g, 102 mmol, 2 eq), followed by anhydrous toluene (400 mL) and the mixture was stirred at RT for 5-7 h under argon. The reaction was diluted with ethyl acetate, washed with water then brine, dried over sodium sulfate, and concentrated. The product was purified by flash chromatography on silica gel (35-50% ethyl acetate/hexanes) to give the desired intermediate (27 g, 99%).

### Step 1e.

To methyl 2-(4-(bis(benzyloxy)phosphoryl)phenyl)-2-((tert-butoxycarbonyl)amino)acetate (27 g, 51.4 mmol) in THF (100 mL)/H₂O (100 mL) was added lithium hydroxide monohydrate (6.48 g, 154 mmol, 3 eq) and the mixture was stirred at RT for 1 h. 2N Hydrochloric acid was added drop wise to obtain pH 2 and extracted with dichloromethane. The organic layer was washed with water, dried over sodium sulfate, and concentrated to give 20 g crude product. The product was purified by flash chromatography on silica gel (10-20% MeOH/DCM) to give the title compound (15.8 g, 60%) as a white solid. ESI-MS m/z 512 (M+H)⁺.

### Step 2. Synthesis of 3-((2-chloro-3,4-dimethoxyphenyl)sulfonyl)-2-oxoimidazolidine-1-carbonyl chloride

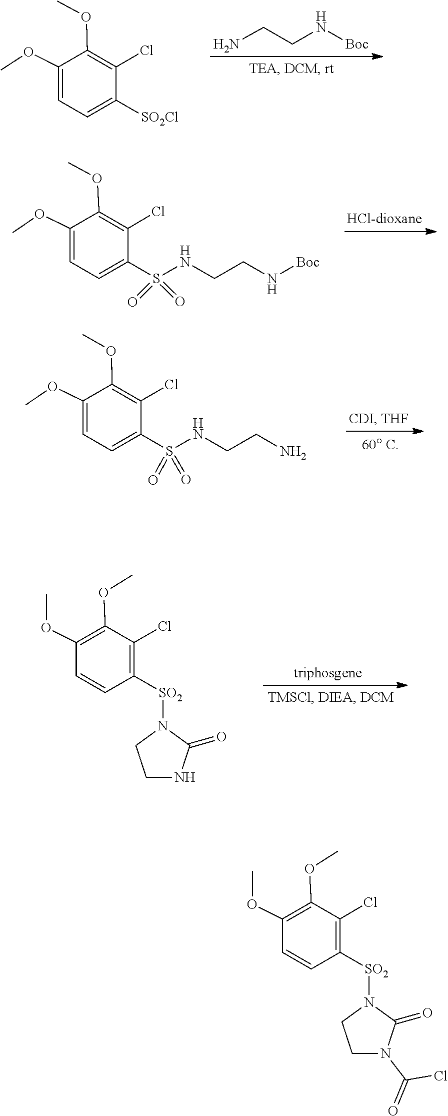 [SMILES: COc1ccc(S(=O)(=O)Cl)c(Cl)c1OC ; CC(C)(C)OC(=O)NCCN ; COc1ccc(S(=O)(=O)NCCNC(=O)OC(C)(C)C)c(Cl)c1OC ; COc1ccc(S(=O)(=O)NCCN)c(Cl)c1OC ; COc1ccc(S(=O)(=O)N2CCNC2=O)c(Cl)c1OC ; COc1ccc(S(=O)(=O)N2CCN(C(=O)Cl)C2=O)c(Cl)c1OC]

### Step 2a.

To a solution of added tert-butyl (2-aminoethyl)carbamate (3.9 mL, 24 mmol, 1.1 equiv.) in DCM (200 mL) was 2-chloro-3,4-dimethoxycyclohexa-2,4-diene-1-sulfonyl chloride (6.1 g, 22 mmol) and TEA (6 mL, 44.0 mmol, 2 equiv.) were added sequentially. The solution was stirred at RT for 2 h. The mixture was concentrated. The crude product was purified by silica gel chromatography (0-5% MeOH/DCM) to yield desired intermediate (8.1 g, 92%)

### Step 2b.

To the crude product in THF (30 mL) at 0° C. was added HCl (4 M in dioxane, 30 mL). The reaction was warmed to RT and stirred for 2 h before being concentrated in vacuo.

### Step 2c.

The crude product was dissolved in THF (400 mL) and TEA (6.3 mL, 45 mmol), and CDI (3.6 g, 22 mmol, 1.1 equiv.) were added sequentially. The mixture was heated to 60° C. overnight and concentrated. The crude product was purified by silica gel chromatography (0-5% MeOH/DCM) to yield desired intermediate (5.1 g, 87.5% over 3 steps).

### Step 2d.

A solution of 1-((2-chloro-3,4-dimethoxyphenyl)sulfonyl)imidazolidin-2-one (3.5 g, 10.9 mmol) and TEA (6 mL, 44 mmol, 4 equiv.) in THF (150 mL) was cooled to 0° C. Triphosgene (1.3 g, 4.36 mmol, 0.4 equiv.) in THF (40 mL) was added and warmed to RT for 1 h. The heterogeneous mixture was filtered through Celite and concentrated in vacuo to provide a sticky brown oil.

### Step 3. Synthesis of 2-(4-(bis(benzyloxy)phosphoryl)phenyl)-2-(3-((2-chloro-3,4-dimethoxyphenyl)sulfonyl)-2-oxoimidazolidine-1-carboxamido)acetic acid

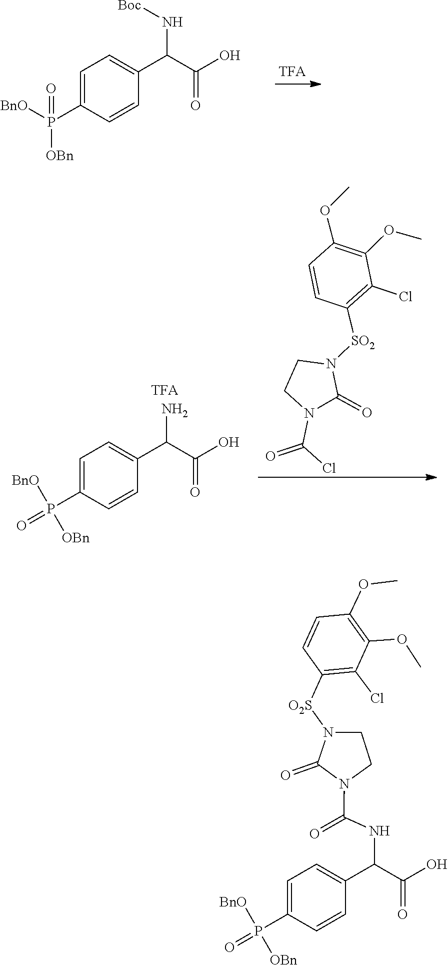 [SMILES: CC(C)(C)OC(=O)NC(C(=O)O)c1ccc(P(=O)(OCc2ccccc2)OCc2ccccc2)cc1 ; NC(C(=O)O)c1ccc(P(=O)(OCc2ccccc2)OCc2ccccc2)cc1 ; COc1ccc(S(=O)(=O)N2CCN(C(=O)Cl)C2=O)c(Cl)c1OC ; COc1ccc(S(=O)(=O)N2CCN(C(=O)NC(C(=O)O)c3ccc(P(=O)(OCc4ccccc4)OCc4ccccc4)cc3)C2=O)c(Cl)c1OC]

### Step 3a.

To a solution of 2-(4-(bis(benzyloxy)phosphoryl)phenyl)-2-((tert-butoxycarbonyl)amino)acetic acid (3 g, 5.86 mmol) in DCM (30.0 mL) at 0° C. was added TFA (7.5 mL). The solution was warmed to RT for 2 h then concentrated.

### Step 3b.

The crude product was dissolved in THF (40 mL)/NaHCO₃ (sat. aq., 40 mL) followed by addition of 4-(2-chloro-3,4-dimethoxybenzyl)-2,3-dioxopiperazine-1-carbonyl chloride (2.47 g, 6.45 mmol, 1.1 equiv.). The reaction was stirred at RT for 2 h. The reaction was diluted with H₂O (5 mL) and acidified until pH 2 with HCl (2 M). Ethyl acetate (200 mL) was added and the organic layer was washed with H₂O (2×50 mL) and brine (1×50 mL), dried (Na₂SO₄), and concentrated. The crude product was purified by silica gel chromatography (0-20% MeOH/DCM) to yield title compound (2.8 g, 62% over 2 steps). ESI-MS m/z 758 (M+H)⁺.

### Step 4. Synthesis of (3R)-3-(2-(3-((2-chloro-3,4-dihydroxyphenyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

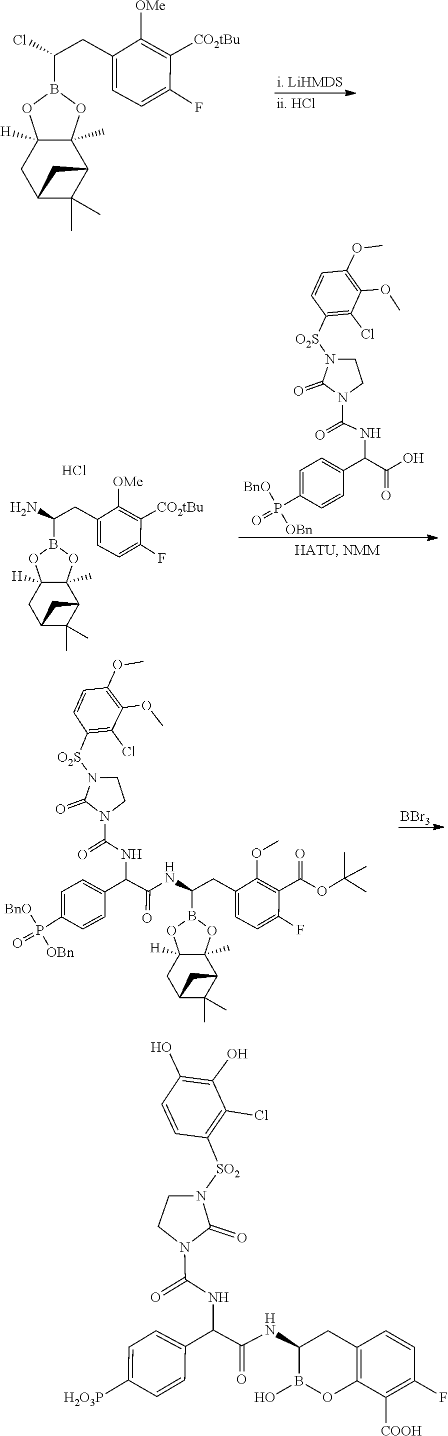

### Step 4a.

A solution of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (5.2 g, 11 mmol) (Example 200, Step 7) in THF (45 mL) was cooled to −78° C. LiHMDS (1 M in THF, 11 mL, 11 mmol, 1.0 equiv.) was added dropwise and the mixture was warmed to RT for 1 h. The solution was cooled to 0° C. and HCl (4 M in dioxane, 11 mL, 44 mmol, 4.0 eq) was added dropwise. The reaction was warmed to RT for 1 h then concentrated. Hexanes (200 mL) was added and stirred at RT overnight. The solid was filtered, washed with hexanes (2×100 mL), and dried to yield desired intermediate tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (5.8 g, 99%).

### Step 4b.

To a solution of tert-butyl 3-((R)-2-amino-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrochloride (2.15 g, 3.7 mmol), 2-(4-(bis(benzyloxy)phosphoryl)phenyl)-2-(3-((2-chloro-3,4-dimethoxyphenyl)sulfonyl)-2-oxoimidazolidine-1-carboxamido)acetic acid (2.8 g, 3.7 mmol, 1 equiv.), and HATU (2.46 g, 6.5 mmol, 1.75 equiv.) in DMA (37 mL) was added NMM (1.2 mL, 11.1 mmol, 3.0 equiv.). The reaction was stirred at RT for 30 min. and diluted with EtOAc (120 mL). The mixture was washed with H₂O (2×100 mL) and brine, dried (Na₂SO₄), filtered, and concentrated. The crude product was purified by silica gel chromatography (0-5% MeOH/DCM) to yield desired tert-butyl 3-((2R)-2-(2-(4-(bis(benzyloxy)phosphoryl)phenyl)-2-(3-((2-chloro-3,4-dimethoxyphenyl)sulfonyl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (1.8 g, 41%) ESI m/z 1187 (M+H)⁺.

### Step 4c.

To a solution of tert-butyl 3-((2R)-2-(2-(4-(bis(benzyloxy)phosphoryl)phenyl)-2-(3-((2-chloro-3,4-dimethoxyphenyl)sulfonyl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate (1.8 g, 1.5 mmol) in DCM (15 mL) at −78° C. was added BBr₃ (1 M in DCM, 15 mL, 2.2 mmol, 10 equiv.). The reaction was warmed to RT overnight then concentrated. The resulting crude product was purified by reverse-phase HPLC to yield (3R)-3-(2-(3-((2-chloro-3,4-dihydroxyphenyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid (150 mg). ESI m z 757 (M+H)⁺.

### Example 506: Synthesis of (R)-3-((R)-2-(3-((2-chloro-3,4-dihydroxyphenyl)sulfonyl)-2-oxoimidazolidine-1-carboxamido)-2-(4-phosphonophenyl)acetamido)-7-fluoro-2-hydroxy-3,4-dihydro-2H-benzo[e][1,2]oxaborinine-8-carboxylic acid

The title compound was prepared in a similar manner to the synthesis of Example 505 with diastereomer separation by reverse phase HPLC purification. ESI-MS m/z 757 (M+H)⁺

### Example 509: Synthesis of (R)-3-((R)-2-(3-((2-chloro-3,4-dihydroxyphenyl)sulfonyl)-2-oxoimidazolidine-1-carboxamido)-2-(4-phosphonophenyl)acetamido)-2-hydroxy-3,4-dihydro-2H-benzo[e][1,2]oxaborinine-8-carboxylic acid

The title compound was prepared in a similar manner to the synthesis of Example 505, utilizing tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-2-methoxybenzoate in place of tert-butyl 3-((S)-2-chloro-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in Step 4a, purified by reverse phase HPLC purification. ESI-MS m/z 738 (M+H)⁺.

### Example 600: Synthesis of (3R)-3-(2-(2-chloro-5-fluoro-3,4-dihydroxyphenyl)-2-(3-(1-((3,5-dihydroxyphenyl)sulfonyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 460, utilizing 3,5-dimethoxybenzenesulfonyl chloride in place of nicotinoyl chloride in Step 2, the title compound was prepared after reversed phase HPLC purification. ESI-MS m/z 810/812 (M+H)⁺/(M+H+2)⁺.

### Example 601: Synthesis of (R)-3-((R)-2-(2-chloro-5-fluoro-3,4-dihydroxyphenyl)-2-(2-oxo-3-(1-(pyridazin-4-ylmethyl)piperidin-4-yl)imidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 492, utilizing pyridazine-4-carbaldehyde in place of 3-pyridinecarbxaldehyde, the title compound was prepared after reversed phase HPLC purification. ESI-MS m/z 730/732 (M+H)⁺/(M+H+2)⁺.

### Example 602: Synthesis of (R)-3-((R)-2-(2-chloro-5-fluoro-3,4-dihydroxyphenyl)-2-(2-oxo-3-(1-(pyrimidin-4-ylmethyl)piperidin-4-yl)imidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 492, utilizing pyrimidine-4-carbaldehyde in place of 3-pyridinecarbxaldehyde, the title compound was prepared after reversed phase HPLC purification. ESI-MS m/z 730/732 (M+H)⁺/(M+H+2)⁺.

### Example 603: Synthesis of (R)-3-((R)-2-(3-(1-(3-(aminomethyl)benzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

### Step 1. Synthesis of 3-fluoro-4,5-dihydroxybenzaldehyde

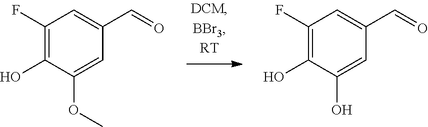

To 3-fluoro-4-hydroxy-5-methoxybenzaldehyde 50 g (294 mmol) in DCM (500 mL) was added a solution of BBr₃ 55 mL (588 mmol, 2 eq) in DCM (250 mL) at −80° C. The reaction mixture was stirred at RT for 6 h. The reaction was quenched by MeOH at −30° C., and evaporated in vacuo. The product was purified by flash chromatography on silica gel (20% MeOH/DCM) to give the desired product, 40 g, 87.3%. ESI-MS m/z 157 (M+H)⁺.

### Step 2. Synthesis of 3-fluoro-5-hydroxy-4-methoxybenzaldehyde

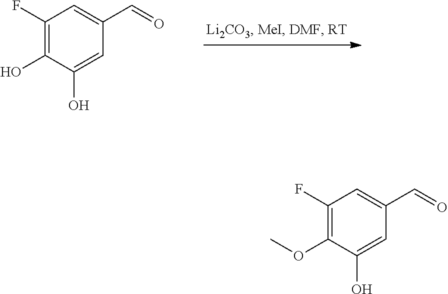

To 3-fluoro-4,5-dihydroxybenzaldehyde 40 g (256 mmol) in DMF (400 mL) was added Li₂CO₃ 28 g (384 mmol, 1.5 eq), followed by MeI 40 g (282 mmol, 1.1 eq) at 0° C. The reaction mixture was stirred at 40° C. for 12 h. The reaction was diluted with EA, washed with NaCl (aq), dried over Na₂SO₄, and evaporated in vacuo. The product was purified by flash chromatography on silica gel (20% EA/PE) to give the desired product, 25 g, 57.5%. ESI-MS m/z 171 (M+H)⁺.

### Step 3: Synthesis of 2-chloro-5-fluoro-3-hydroxy-4-methoxybenzaldehyde

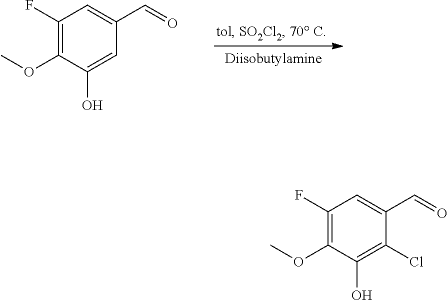

To a solution of 3-fluoro-5-hydroxy-4-methoxybenzaldehyde 25 g (147 mmol) in Toluene was added diisobutylamine 2.1 mL (13 mmol, 0.09 eq). The mixture was heated to 70° C. in oil bath, and added sulfuryl chloride 14 mL (169 mmol, 1.15 eq) at 70° C. The reaction mixture was stirred for 2 h at 70° C. The resulting mixture was concentrated in vacuo, diluted with water, extracted with EA, dried over Na₂SO₄, concentrated in vacuo. The product was purified by flash chromatography on silica gel (10% EA/PE) to give the desired product, 26 g, 86.6%. ESI-MS m/z 205 (M+H)⁺.

### Step 4: Synthesis of 2-chloro-5-fluoro-3,4-dimethoxybenzaldehyde

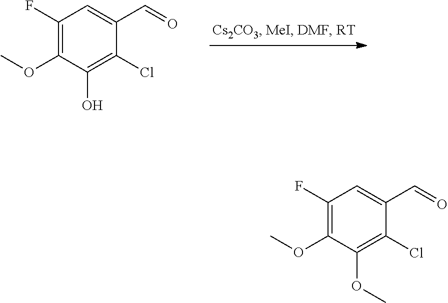

To a solution of 2-chloro-5-fluoro-3-hydroxy-4-methoxybenzaldehyde 26 g (127 mmol) in DMF was added Cs₂CO₃ 62 g (191 mmol, 1.5 eq), followed by MeI 21.6 g (152 mmol, 1.2 eq) at 0° C. The mixture was stirred for 5 h at RT. The reaction mixture was diluted with EA, washed with water, dried over Na₂SO₄, concentrated in vacuo. The product was purified by flash chromatography on silica gel (10% EA/PE) to give the desired product, 25 g, 90.5%. ESI-MS m/z 219 (M+H)⁺.

### Step 5: Synthesis of 2-((tert-butoxycarbonyl)amino)-2-(2-chloro-5-fluoro-3,4-dimethoxyphenyl)acetic acid

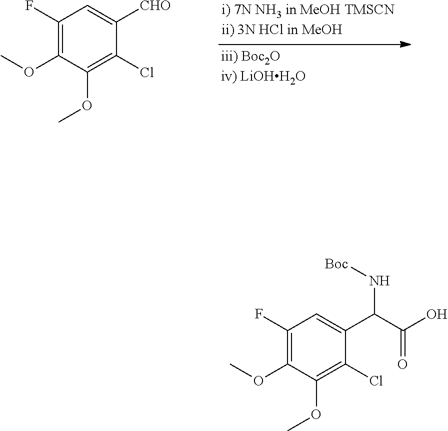 [SMILES: COc1c(F)cc(C=O)c(Cl)c1OC ; COc1c(F)cc(C(NC(=O)OC(C)(C)C)C(=O)O)c(Cl)c1OC]

To 2-chloro-5-fluoro-3,4-dimethoxybenzaldehyde 25 g (115 mmol) at 0° C. was added 7 N ammonia in methanol (550 mL), followed by trimethylsilyl cyanide 21.5 mL (172.5 mmol, 1.5 eq), stirred at 45° C. for 7 h and concentrated in vacuo. The crude product was dissolved in 3 N hydrochloric acid in methanol (450 mL), stirred at 50° C. for 18 h and concentrated in vacuo to give the HCl salt. The reaction was slurried in tetrahydrofuran (500 mL) and cooled at 0° C. Triethylamine 48 mL (345 mmol, 3 eq) was added, followed by di-tert-butyl dicarbonate 37.5 g (172.5 mmol, 1.5 eq), warmed at RT for 1 h and concentrated in vacuo. The product was purified by flash chromatography on silica gel (20-30% ethyl acetate/hexanes) to give the desired product, 30 g. methyl 2-((tert-butoxycarbonyl)amino)-2-(2-chloro-5-fluoro-3,4-dimethoxyphenyl)acetate 30 g (79.5 mmol) was dissolved in tetrahydrofuran (260 mL)/H₂O (260 mL), followed by lithium hydroxide monohydrate 5 g (119 mmol, 1.5 eq) and was stirred at RT for 2 h and concentrated. The product was evaporated in vacuo, adjusted to pH=3 with HCl (1 M), extracted with EA, dried over Na₂SO₄, concentrated in vacuo. The product was purified by flash chromatography on silica gel (40% EA/PE) to give the desired product, 24 g, 83.3%. ESI-MS m/z 364 (M+H)⁺.

### Step 6: Synthesis of tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

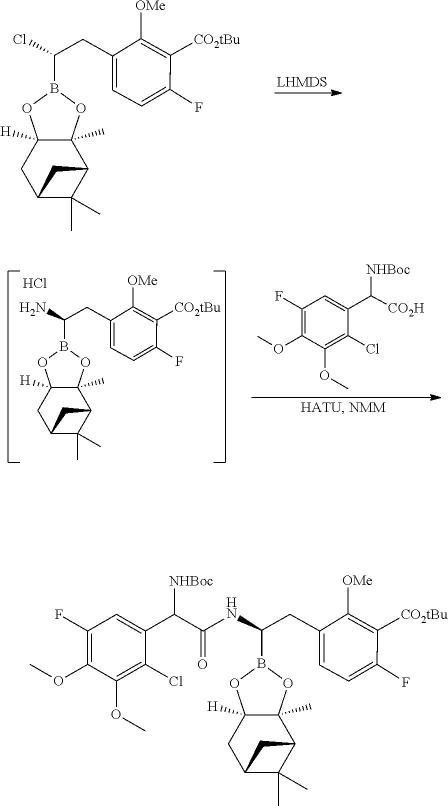

By following the General procedure C, the chloride (prepared as previous reported, WO 2014/089365) was treated with LiHMDS and 4 M HCl in dioxane, and then coupled 2-((tert-butoxycarbonyl)amino)-2-(2-chloro-5-fluoro-3,4-dimethoxyphenyl)acetic acid in the presence of HATU and NMM, yielding the title compound. ESI-MS m/z 793 (M+H)⁺.

### Step 7: Synthesis of tert-butyl 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate

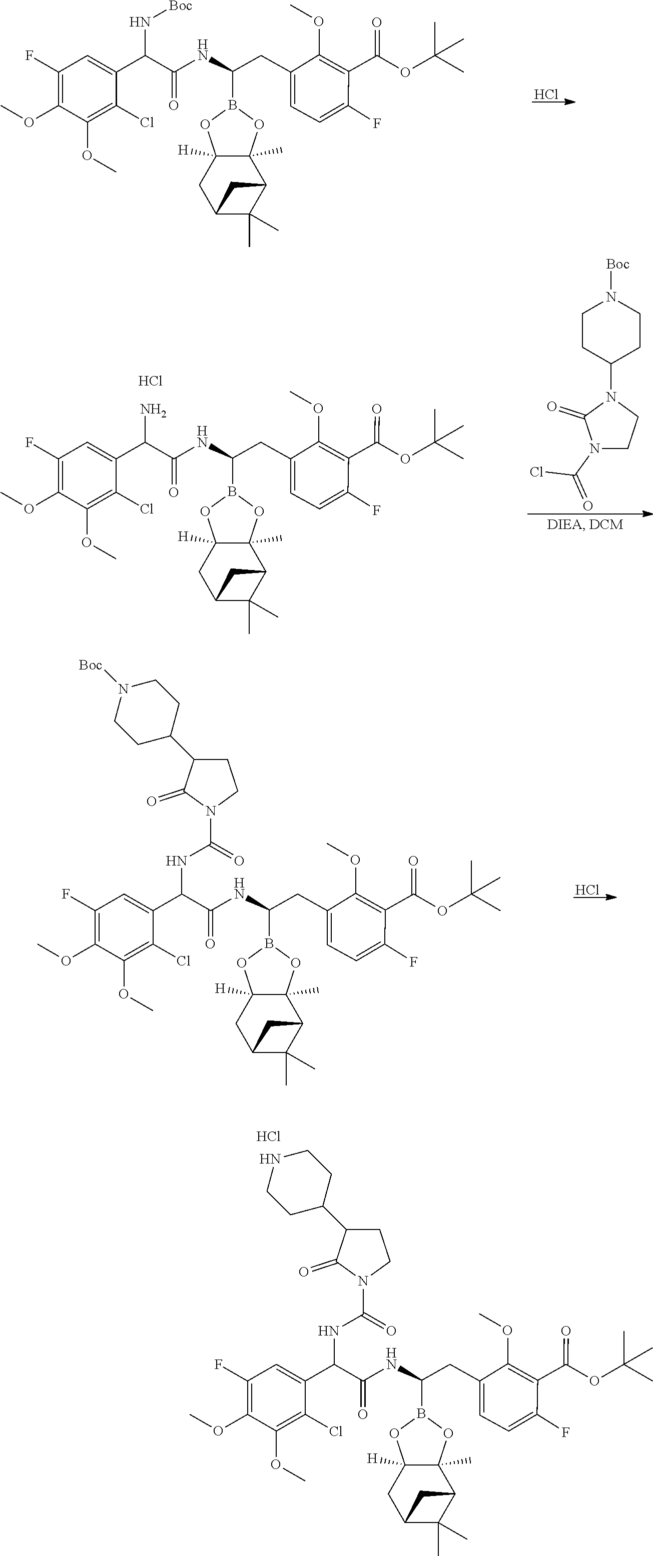

### Step 7a.

To tert-butyl 3-((2R)-2-(2-((tert-butoxycarbonyl)amino)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate 40 g (25 mmol) at 0° C. was added 1 N hydrochloric acid in diethyl ether (400 mL) and warmed at RT for 18 h. The reaction was concentrated in vacuo to give tert-butyl 3-((2R)-2-(2-amino-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate hydrogen chloride, 32 g, 91.4%. ESI-MS m/z 693 (M+H)⁺.

### Step 7b.

The crude product was dissolved in DCM followed by addition of tert-butyl 4-(3-(chlorocarbonyl)-2-oxoimidazolidin-1-yl)piperidine-1-carboxylate (1.1 equiv.) and TEA (3 equiv.) The reaction was stirred at RT for 2 h. The reaction was diluted with H₂O. DCM was added and the organic layer was washed with H₂O (2×50 mL) and brine (1×50 mL), dried (Na₂SO₄), and concentrated. ESI-MS m/z 988 (M+H)⁺.

### Step 7c.

To tert-butyl 4-(3-((2-(((R)-2-(3-(tert-butoxycarbonyl)-4-fluoro-2-methoxyphenyl)-1-((3aS,4S,6S,7aR)-3a,5,5-trimethylhexahydro-4,6-methanobenzo[d][1,3,2]dioxaborol-2-yl)ethyl)amino)-1-(2-chloro-5-fluoro-3,4-dimethoxyphenyl)-2-oxoethyl)carbamoyl)-2-oxoimidazolidin-1-yl)piperidine-1-carboxylate at 0° C. was added 1 N hydrochloric acid in diethyl ether and warmed at RT for 18 h. The reaction was concentrated in vacuo to give tert-butyl 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 888 (M+H)⁺.

### Step 8: Synthesis of (R)-3-((R)-2-(3-(1-(3-(aminomethyl)benzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

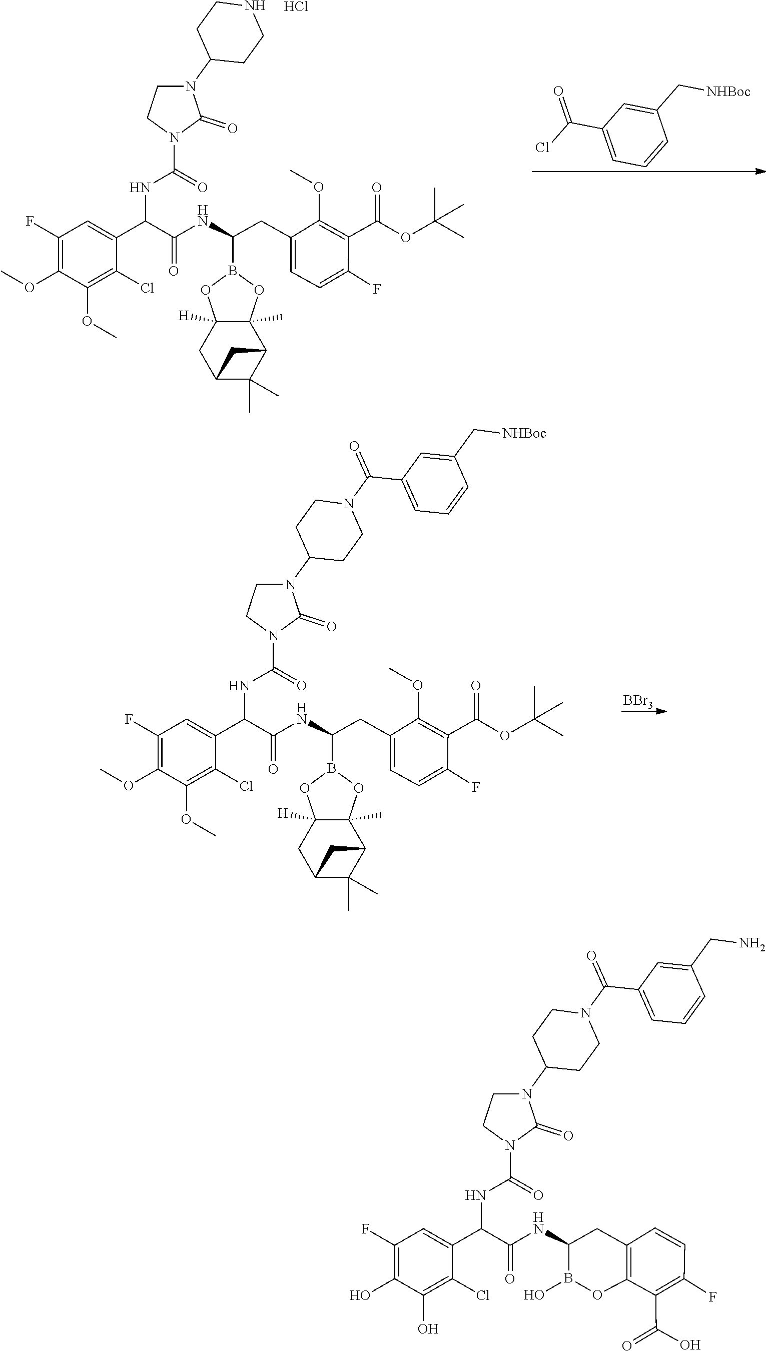

### Step 8a.

Give tert-butyl 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate was dissolved in DCM followed by addition of tert-butyl (3-(chlorocarbonyl)benzyl)carbamate (1.1 equiv.) and TEA (3 equiv.) The reaction was stirred at RT for 2 h. The reaction was diluted with H₂O. DCM was added and the organic layer was washed with H₂O (2×50 mL) and brine (1×50 mL), dried (Na₂SO₄), and concentrated to give tert-butyl 3-((2R)-2-(2-(3-(1-(3-(((tert-butoxycarbonyl)amino)methyl)benzoyl)piperidin-4-yl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 1121 (M+H)⁺.

### Step 8b.

To a solution of tert-butyl 3-((2R)-2-(2-(3-(1-(3-(((tert-butoxycarbonyl)amino)methyl)benzoyl)piperidin-4-yl)-2-oxoimidazolidine-1-carboxamido)-2-(2-chloro-5-fluoro-3,4-dimethoxyphenyl)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in DCM at −78° C. was added BBr₃ (10 equiv.). The reaction was warmed to RT overnight then concentrated. The resulting crude product was purified by reverse-phase HPLC to yield (3R)-3-(2-(3-(1-(3-(aminomethyl)benzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid. ESI m/z 771 (M+H)⁺.

### Example 604: Synthesis of 3R)-3-(2-(3-(1-(4-(aminomethyl)benzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 603, utilizing tert-butyl (4-(chlorocarbonyl)benzyl)carbamate in place of tert-butyl (3-(chlorocarbonyl)benzyl)carbamate in Step 8. After reversed phase HPLC purification, the title compound was collected as the second eluting peak. ESI-MS m/z 771 (M+H)⁺.

### Example 605: Synthesis of (3R)-3-(2-(2-chloro-5-fluoro-3,4-dihydroxyphenyl)-2-(3-(1-(3-hydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

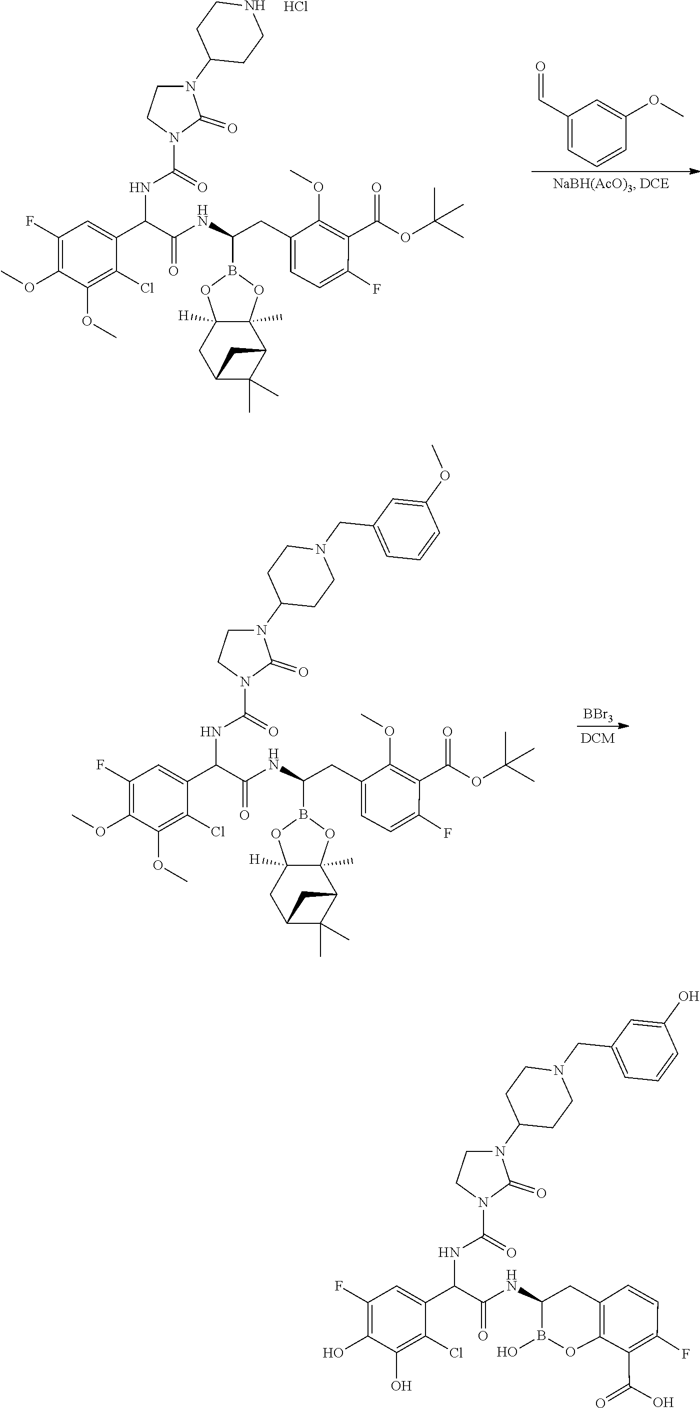

Intermediate tert-butyl 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate was prepared in a similar manner to the synthesis of Example 603 (Step 1 through Step 7).

### Step 8a.

A solution of tert-butyl 3-((2R)-2-(2-(2-chloro-5-fluoro-3,4-dimethoxyphenyl)-2-(2-oxo-3-(piperidin-4-yl)imidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate and 3-methoxybenzaldehyde (1.1 equiv.) in DCE followed by addition of NaBH(OAc)₃ (3 equiv.) The reaction was stirred at RT overnight. The reaction was diluted with H₂O. DCM was added and the organic layer was washed with H₂O and brine, dried (Na₂SO₄), and concentrated to give tert-butyl 3-((2R)-2-(2-(2-chloro-5-fluoro-3,4-dimethoxyphenyl)-2-(3-(1-(3-methoxybenzyl)piperidin-4-yl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate. ESI-MS m/z 1008 (M+H)⁺.

### Step 8b.

To a solution of tert-butyl 3-((2R)-2-(2-(2-chloro-5-fluoro-3,4-dimethoxyphenyl)-2-(3-(1-(3-methoxybenzyl)piperidin-4-yl)-2-oxoimidazolidine-1-carboxamido)acetamido)-2-((3aS,4S,6S,7aR)-3a,5,5-trimethylhexahydro-4,6-methanobenzo[d][1,3,2]dioxaborol-2-yl)ethyl)-6-fluoro-2-methoxybenzoate in DCM at −78° C. was added BBr₃ (10 equiv.). The reaction was warmed to RT overnight then concentrated. The resulting crude product was purified by reverse-phase HPLC to yield (3R)-3-(2-(2-chloro-5-fluoro-3,4-dihydroxyphenyl)-2-(3-(1-(3-hydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid. ESI m/z 744 (M+H)⁺.

### Example 606: Synthesis of (3R)-3-(2-(2-chloro-5-fluoro-3,4-dihydroxyphenyl)-2-(3-(1-(4-fluoro-3-hydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing 4-fluoro-3-methoxybenzaldehyde in place of 3-methoxybenzaldehyde in Step 8. After reversed phase HPLC purification, the title compound was collected as the second eluting peak. ESI-MS m/z 762 (M+H)⁺.

### Example 607: Synthesis of (R)-3-((R)-2-(2-chloro-5-fluoro-3,4-dihydroxyphenyl)-2-(3-(1-(3-fluoro-5-hydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing 3-fluoro-5-methoxybenzaldehyde in place of 3-methoxybenzaldehyde in Step 8. After reversed phase HPLC purification, the title compound was collected as the second eluting peak. ESI-MS m/z 762 (M+H)⁺.

### Example 608: Synthesis of (R)-3-((R)-2-(2-chloro-5-fluoro-3,4-dihydroxyphenyl)-2-(3-(1-(2-fluoro-5-hydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing 2-fluoro-5-methoxybenzaldehyde in place of 3-methoxybenzaldehyde in Step 8. After reversed phase HPLC purification, the title compound was collected as the second eluting peak. ESI-MS m/z 762 (M+H)⁺.

### Example 609: Synthesis of (3R)-3-(2-(2-chloro-5-fluoro-3,4-dihydroxyphenyl)-2-(3-(1-(4-hydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing 4-methoxybenzaldehyde in place of 3-methoxybenzaldehyde in Step 8. After reversed phase HPLC purification, the title compound was collected as the second eluting peak. ESI-MS m/z 744 (M+H)⁺.

### Example 610: Synthesis of (3R)-3-(2-(2-chloro-5-fluoro-3,4-dihydroxyphenyl)-2-(3-(1-(3-fluoro-4-hydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing 3-fluoro-4-methoxybenzaldehyde in place of 3-methoxybenzaldehyde in Step 8. After reversed phase HPLC purification, the title compound was collected as the second eluting peak. ESI-MS m/z 762 (M+H)⁺.

### Example 611: Synthesis of (3R)-3-(2-(2-chloro-5-fluoro-3,4-dihydroxyphenyl)-2-(3-(1-(2-fluoro-4-hydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing 2-fluoro-4-methoxybenzaldehyde in place of 3-methoxybenzaldehyde in Step 8. After reversed phase HPLC purification, the title compound was collected as the second eluting peak. ESI-MS m/z 762 (M+H)⁺.

### Example 612: Synthesis of (3R)-3-(2-(2-chloro-5-fluoro-3,4-dihydroxyphenyl)-2-(3-(1-(3,5-dihydroxybenzyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing 3,5-dimethoxybenzaldehyde in place of 3-methoxybenzaldehyde in Step 8. After reversed phase HPLC purification, the title compound was collected as the second eluting peak. ESI-MS m/z 760 (M+H)⁺.

### Example 613: Synthesis of (R)-3-((R)-2-(3-(1-(3-(aminomethyl)benzyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing tert-butyl (3-formylbenzyl)carbamate in place of 3-methoxybenzaldehyde in Step 8. After reversed phase HPLC purification, the title compound was collected as the second eluting peak. ESI-MS m/z 757 (M+H)⁺.

### Example 614: Synthesis of (R)-3-((R)-2-(2-chloro-5-fluoro-3,4-dihydroxyphenyl)-2-(3-(1-((5-hydroxypyridin-3-yl)methyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing 5-methoxynicotinaldehyde in place of 3-methoxybenzaldehyde in Step 8. After reversed phase HPLC purification, the title compound was collected as the second eluting peak. ESI-MS m/z 745 (M+H)⁺.

### Example 615: Synthesis of (R)-3-((R)-2-(3-(1-(3-(aminomethyl)-5-hydroxybenzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 603, utilizing tert-butyl (3-(chlorocarbonyl)-5-methoxybenzyl)carbamate in place of tert-butyl (3-(chlorocarbonyl)benzyl)carbamate in Step 8. After reversed phase HPLC purification, the title compound was collected as the second eluting peak. ESI-MS m/z 787 (M+H)⁺.

### Example 616: Synthesis of (3R)-3-(2-(3-(1-(4-(aminomethyl)-3-hydroxybenzoyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 603, utilizing tert-butyl (4-(chlorocarbonyl)-2-methoxybenzyl)carbamate in place of tert-butyl (3-(chlorocarbonyl)benzyl)carbamate in Step 8. After reversed phase HPLC purification, the title compound was collected as the second eluting peak. ESI-MS m/z 787 (M+H)⁺.

### Example 617: Synthesis of (R)-3-((R)-2-(3-(1-(3-(aminomethyl)-5-hydroxybenzyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing tert-butyl (3-formyl-5-methoxybenzyl)carbamate in place of 3-methoxybenzaldehyde in Step 8. After reversed phase HPLC purification, the title compound was collected as the second eluting peak. ESI-MS m/z 773 (M+H)⁺.

### Example 618: Synthesis of (R)-3-((R)-2-(3-(1-((6-aminopyridin-3-yl)methyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

The title compound was prepared in a similar manner to the synthesis of Example 605, utilizing tert-butyl (5-formylpyridin-2-yl)carbamate in place of 3-methoxybenzaldehyde in Step 8. After reversed phase HPLC purification, the title compound was collected as the second eluting peak. ESI-MS m/z 744 (M+H)⁺.

### Example 619: Synthesis of (R)-3-((R)-2-(3-(1-((2-aminopyridin-4-yl)methyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 492, utilizing tert-butyl (4-formylpyridin-2-yl)carbamate in place of 3-pyridinecarbxaldehyde, the title compound was prepared after reversed phase HPLC purification. ESI-MS m/z 744/746 (M+H)⁺/(M+H+2)⁺.

### Example 620: Synthesis of (R)-3-((S)-2-(3-(1-((1H-benzo[d][1,2,3]triazol-5-yl)sulfonyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid, and

### Example 621: Synthesis of (R)-3-((R)-2-(3-(1-((1H-benzo[d][1,2,3]triazol-5-yl)sulfonyl)piperidin-4-yl)-2-oxoimidazolidine-1-carboxamido)-2-(2-chloro-5-fluoro-3,4-dihydroxyphenyl)acetamido)-7-fluoro-2-hydroxy-3,4-dihydro-2H-benzo[e][1,2]oxaborinine-8-carboxylic acid

In a similar manner to the synthesis of Example 460, utilizing 1H-benzo[d][1,2,3]triazole-5-sulfonyl chloride in place of nicotinoyl chloride in Step 2, Example 620 and Example 621 were prepared after reversed phase HPLC purification as first eluting peak and second eluting peak respectively. ESI-MS m/z 819/821 (M+H)⁺/(MH+2)⁺.

### Example 622: Synthesis of (R)-3-((R)-2-(3-(1-((1H-benzo[d][1,2,3]triazol-5-yl)sulfonyl)piperidin-4-yl)-2-oxoimidazolidine-1-carboxamido)-2-(2,5-difluoro-3,4-dihydroxyphenyl)acetamido)-7-fluoro-2-hydroxy-3,4-dihydro-2H-benzo[e][1,2]oxaborinine-8-carboxylic acid

Ina similar manner to the synthesis of Example 621, utilizing commercially available 2,5-difluoro-3,4-dimethoxybenzaldehyde in place of 2-chloro-5-fluoro-3,4-dimethoxybenzaldehyde for the Strecker reaction to make 2-((tert-butoxycarbonyl)amino)-2-(2,5-difluoro-3,4-dimethoxyphenyl)acetic acid, Example 622 was prepared after reversed phase HPLC purification. ESI-MS m/z 803/805 (M+H)⁺/(M+H+2)⁺.

### Example 623: Synthesis of (R)-3-((R)-2-(2,5-difluoro-3,4-dihydroxyphenyl)-2-(3-(1-(3,5-dihydroxybenzoyl)piperidin-4-yl)-2-oxoimidazolidine-1-carboxamido)acetamido)-7-fluoro-2-hydroxy-3,4-dihydro-2H-benzo[e][1,2]oxaborinine-8-carboxylic acid

In a similar manner to the synthesis of Example 622, utilizing 3,5-dimethoxybenzoyl chloride in place of 1H-benzo[d][1,2,3]triazole-5-sulfonyl chloride, the title compound was prepared after reversed phase HPLC purification. ESI-MS m/z 758/760 (M+H)⁺/(M+H+2)⁺.

Exemplary compounds are found in Tables 1-4.

TABLE 1

| | | | | ESI-MS |
|---|---|---|---|---|
| | | | | (m/z) for |
| Ex. | Structure | Name | MW | [M + H]⁺ |
| 1 | 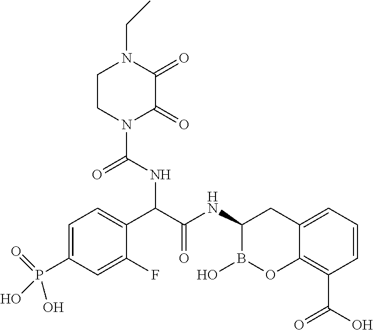 [SMILES: CCN1CCN(C(=O)NC(C(=O)N[C@H]2Cc3cccc(C(=O)O)c3OB2O)c2ccc(P(=O)(O)O)cc2F)C(=O)C1=O] | (3R)-3-(2-(4-ethyl-2,3- dioxopiperazine-1-carboxamido)- 2-(2-fluoro-4- phosphonophenyl)acetamido)-2- hydroxy-3,4-dihydro-2H- benzo[e][1,2]oxaborinine-8- carboxylic acid | 606.26 | 607 |
| 2 | 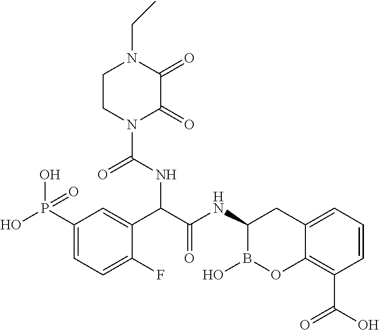 [SMILES: CCN1CCN(C(=O)NC(C(=O)N[C@H]2Cc3cccc(C(=O)O)c3OB2O)c2cc(P(=O)(O)O)ccc2F)C(=O)C1=O] | (3R)-3-(2-(4-ethyl-2,3- dioxopiperazine-1-carboxamido)- 2-(2-fluoro-5- phosphonophenyl)acetamido)-2- hydroxy-3,4-dihydro-2H- benzo[e][1,2]oxaborinine-8- carboxylic acid | 606.26 | 607 |
| 3 | 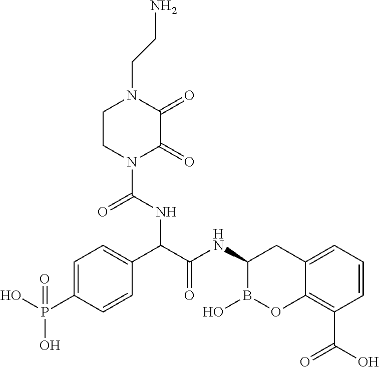 [SMILES: NCCN1CCN(C(=O)NC(C(=O)N[C@H]2Cc3cccc(C(=O)O)c3OB2O)c2ccc(P(=O)(O)O)cc2)C(=O)C1=O] | (3R)-3-(2-(4-(2-aminoethyl)-2,3- dioxopiperazine-1-carboxamido)- 2-(4- phosphonophenyl)acetamido)-2- hydroxy-3,4-dihydro-2H- benzo[e][1,2]oxaborinine-8- carboxylic acid | 603.29 | 604 |
| 4 | 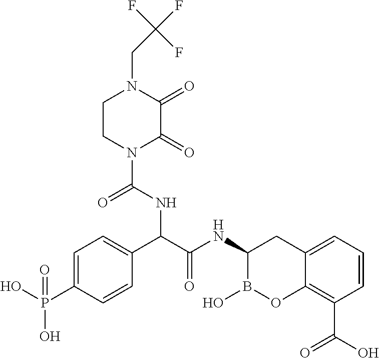 [SMILES: O=C(O)c1cccc2c1OB(O)[C@@H](NC(=O)C(NC(=O)N1CCN(CC(F)(F)F)C(=O)C1=O)c1ccc(P(=O)(O)O)cc1)C2] | (3R)-3-(2-(2,3-dioxo-4-(2,2,2- trifluoroethyl)piperazine-1- carboxamido)-2-(4- phosphonophenyl)acetamido)-2- hydroxy-3,4-dihydro-2H- benzo[e][1,2]oxaborinine-8- carboxylic acid | 642.24 | 643 |
| 5 | 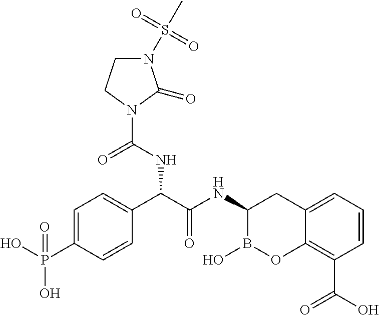 [SMILES: CS(=O)(=O)N1CCN(C(=O)N[C@H](C(=O)N[C@H]2Cc3cccc(C(=O)O)c3OB2O)c2ccc(P(=O)(O)O)cc2)C1=O] | (R)-2-hydroxy-3-((S)-2-(3- (methylsulfonyl)-2- oxoimidazolidine-1- carboxamido)-2-(4- phosphonophenyl)acetamido)- 3,4-dihydro-2H- benzo[e][1,2]oxaborinine-8- carboxylic acid | 610.29 | 611 |
| 6 | 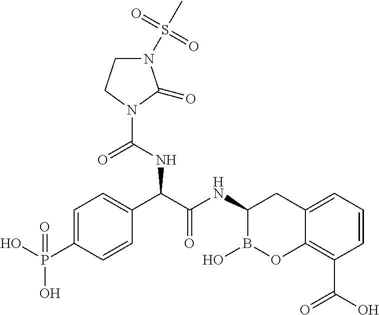 [SMILES: CS(=O)(=O)N1CCN(C(=O)N[C@@H](C(=O)N[C@H]2Cc3cccc(C(=O)O)c3OB2O)c2ccc(P(=O)(O)O)cc2)C1=O] | (R)-2-hydroxy-3-((R)-2-(3- (methylsulfonyl)-2- oxoimidazolidine-1- carboxamido)-2-(4- phosphonophenyl)acetamido)- 3,4-dihydro-2H- benzo[e][1,2]oxaborinine-8- carboxylic acid | 610.29 | 611 |
| 7 | 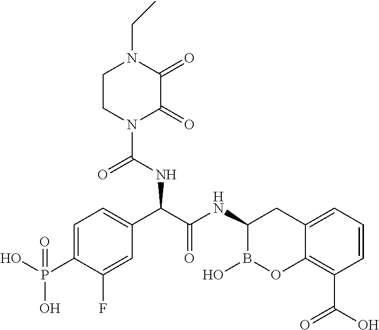 [SMILES: CCN1CCN(C(=O)N[C@@H](C(=O)N[C@H]2Cc3cccc(C(=O)O)c3OB2O)c2ccc(P(=O)(O)O)c(F)c2)C(=O)C1=O] | (R)-3-((R)-2-(4-ethyl-2,3- dioxopiperazine-1-carboxamido)- 2-(3-fluoro-4- phosphonophenyl)acetamido)-2- hydroxy-3,4-dihydro-2H- benzo[e][1,2]oxaborinine-8- carboxylic acid | 606.26 | 607 |
| 8 | 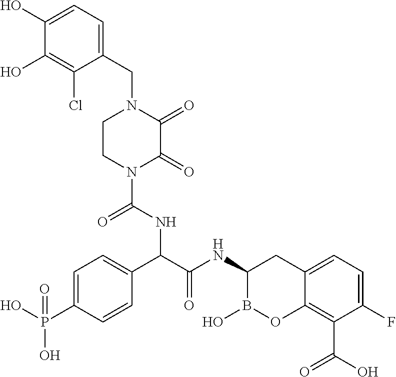 [SMILES: O=C(O)c1c(F)ccc2c1OB(O)[C@@H](NC(=O)C(NC(=O)N1CCN(Cc3ccc(O)c(O)c3Cl)C(=O)C1=O)c1ccc(P(=O)(O)O)cc1)C2] | (3R)-3-(2-(4-(2-chloro-3,4- dihydroxybenzyl)-2,3- dioxopiperazine-1-carboxamido)- 2-(4- phosphonophenyl)acetamido)-7- fluoro-2-hydroxy-3,4-dihydro- 2H-benzo[e][1,2]oxaborinine-8- carboxylic acid | 734.77 | 735 |
| 9 | 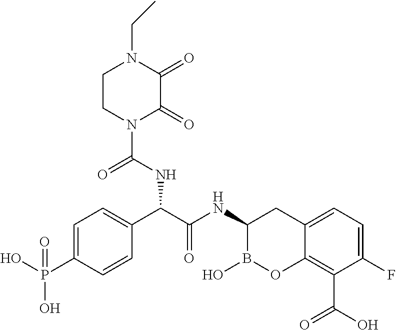 [SMILES: CCN1CCN(C(=O)N[C@H](C(=O)N[C@H]2Cc3ccc(F)c(C(=O)O)c3OB2O)c2ccc(P(=O)(O)O)cc2)C(=O)C1=O] | (R)-3-((S)-2-(4-ethyl-2,3- dioxopiperazine-1-carboxamido)- 2-(4- phosphonophenyl)acetamido)-7- fluoro-2-hydroxy-3,4-dihydro- 2H-benzo[e][1,2]oxaborinine-8- carboxylic acid | 606.26 | 607 |
| 10 | 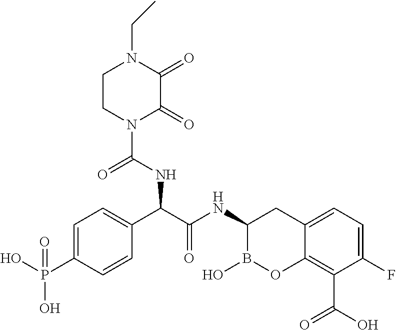 [SMILES: CCN1CCN(C(=O)N[C@@H](C(=O)N[C@H]2Cc3ccc(F)c(C(=O)O)c3OB2O)c2ccc(P(=O)(O)O)cc2)C(=O)C1=O] | (R)-3-((R)-2-(4-ethyl-2,3- dioxopiperazine-1-carboxamido)- 2-(4- phosphonophenyl)acetamido)-7- fluoro-2-hydroxy-3,4-dihydro- 2H-benzo[e][1,2]oxaborinine-8- carboxylic acid | 606.26 | 607 |
| 11 | 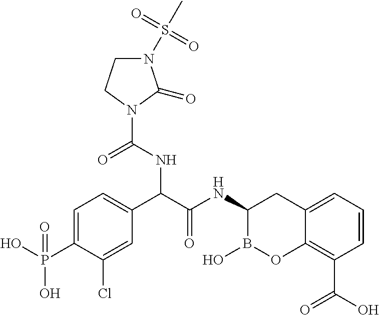 [SMILES: CS(=O)(=O)N1CCN(C(=O)NC(C(=O)N[C@H]2Cc3cccc(C(=O)O)c3OB2O)c2ccc(P(=O)(O)O)c(Cl)c2)C1=O] | (3R)-3-(2-(3-chloro-4- phosphonophenyl)-2-(3- (methylsulfonyl)-2- oxoimidazolidine-1- carboxamido)acetamido)-2- hydroxy-3,4-dihydro-2H- benzo[e][1,2]oxaborinine-8- carboxylic acid | 644.74 | 645 |
| 12 | 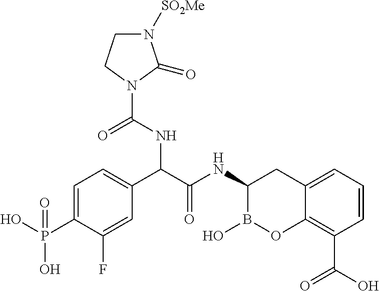 [SMILES: CS(=O)(=O)N1CCN(C(=O)NC(C(=O)N[C@H]2Cc3cccc(C(=O)O)c3OB2O)c2ccc(P(=O)(O)O)c(F)c2)C1=O] | (3R)-3-(2-(3-fluoro-4- phosphonophenyl)-2-(3- (methylsulfonyl)-2- oxoimidazolidine-1- carboxamido)acetamido)-2- hydroxy-3,4-dihydro-2H- benzo[e][1,2]oxaborinine-8- carboxylic acid | 628.28 | 629 |
| 13 | 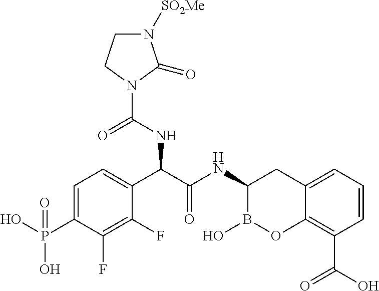 [SMILES: CS(=O)(=O)N1CCN(C(=O)N[C@@H](C(=O)N[C@H]2Cc3cccc(C(=O)O)c3OB2O)c2ccc(P(=O)(O)O)c(F)c2F)C1=O] | (R)-3-((R)-2-(2,3-difluoro-4- phosphonophenyl)-2-(3- (methylsulfonyl)-2- oxoimidazolidine-1- carboxamido)acetamido)-2- hydroxy-3,4-dihydro-2H- benzo[e][1,2]oxaborinine-8- carboxylic acid | 646.27 | 647 |
| 14 | 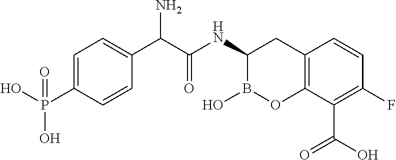 [SMILES: NC(C(=O)N[C@H]1Cc2ccc(F)c(C(=O)O)c2OB1O)c1ccc(P(=O)(O)O)cc1] | (3R)-3-(2-amino-2-(4- phosphonophenyl)acetamido)-7- fluoro-2-hydroxy-3,4-dihydro- 2H-benzo[e][1,2]oxaborinine-8- carboxylic acid | 438.11 | 438 |
| 15 | 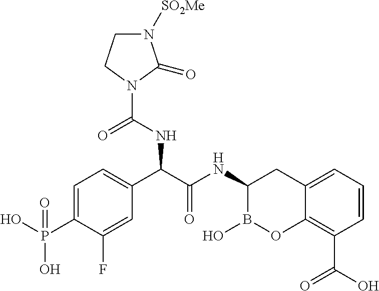 [SMILES: CS(=O)(=O)N1CCN(C(=O)N[C@@H](C(=O)N[C@H]2Cc3cccc(C(=O)O)c3OB2O)c2ccc(P(=O)(O)O)c(F)c2)C1=O] | (R)-3-((R)-2-(3-fluoro-4- phosphonophenyl)-2-(3- (methylsulfonyl)-2- oxoimidazolidine-1- carboxamido)acetamido)-2- hydroxy-3,4-dihydro-2H- benzo[e][1,2]oxaborinine-8- carboxylic acid | 628.28 | 629 |
| 16 | 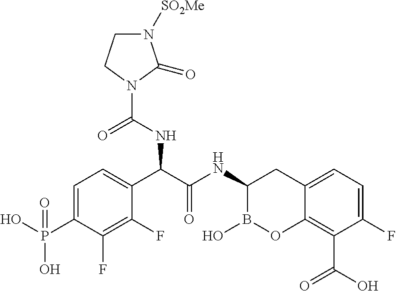 [SMILES: CS(=O)(=O)N1CCN(C(=O)N[C@@H](C(=O)N[C@H]2Cc3ccc(F)c(C(=O)O)c3OB2O)c2ccc(P(=O)(O)O)c(F)c2F)C1=O] | (R)-3-((R)-2-(2,3-difluoro-4- phosphonophenyl)-2-(3- (methylsulfonyl)-2- oxoimidazolidine-1- carboxamido)acetamido)-7- fluoro-2-hydroxy-3,4-dihydro- 2H-benzo[e][1,2]oxaborinine-8- carboxylic acid | 664.26 | 665 |
| 17 | 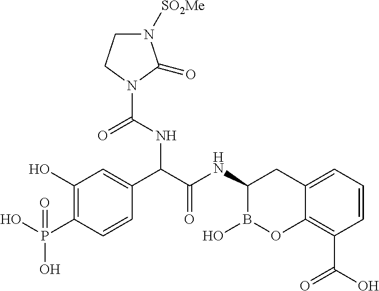 [SMILES: CS(=O)(=O)N1CCN(C(=O)NC(C(=O)N[C@H]2Cc3cccc(C(=O)O)c3OB2O)c2ccc(P(=O)(O)O)c(O)c2)C1=O] | (3R)-2-hydroxy-3-(2-(3-hydroxy- 4-phosphonophenyl)-2-(3- (methylsulfonyl)-2- oxoimidazolidine-1- carboxamido)acetamido)-3,4- dihydro-2H- benzo[e][1,2]oxaborinine-8- carboxylic acid | 626.29 | 627 |
| 18 | 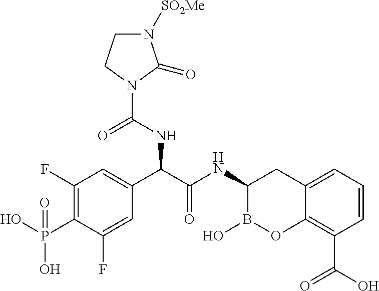 [SMILES: CS(=O)(=O)N1CCN(C(=O)N[C@@H](C(=O)N[C@H]2Cc3cccc(C(=O)O)c3OB2O)c2cc(F)c(P(=O)(O)O)c(F)c2)C1=O] | (R)-3-((R)-2-(3,5-difluoro-4- phosphonophenyl)-2-(3- (methylsulfonyl)-2- oxoimidazolidine-1- carboxamido)acetamido)-2- hydroxy-3,4-dihydro-2H- benzo[e][1,2]oxaborinine-8- carboxylic acid | 646.28 | 647 |
| 19 | 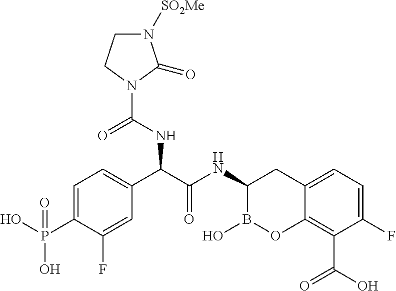 [SMILES: CS(=O)(=O)N1CCN(C(=O)N[C@@H](C(=O)N[C@H]2Cc3ccc(F)c(C(=O)O)c3OB2O)c2ccc(P(=O)(O)O)c(F)c2)C1=O] | (R)-7-fluoro-3-((R)-2-(3-fluoro- 4-phosphonophenyl)-2-(3- (methylsulfonyl)-2- oxoimidazolidine-1- carboxamido)acetamido)-2- hydroxy-3,4-dihydro-2H- benzo[e][1,2]oxaborinine-8- carboxylic acid | 646.28 | 647 |
| 20 | 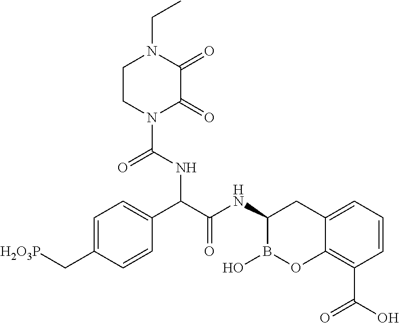 | (3R)-3-(2-(4-ethyl-2,3- dioxopiperazine-1-carboxamido)- 2-(4- (phosphonomethyl)phenyl) acetamido)-2- hydroxy-3,4-dihydro- 2H-benzo[e][1,2]oxaborinine-8- carboxylic acid | 602.30 | 603 |
| 21 | 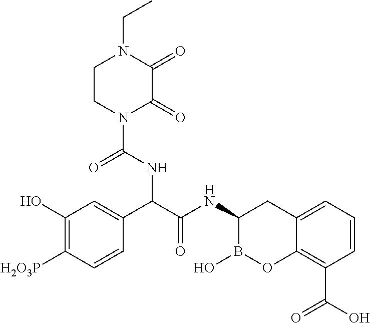 | (3R)-3-(2-(4-ethyl-2,3- dioxopiperazine-1-carboxamido)- 2-(3-hydroxy-4- phosphonophenyl)acetamido)-2- hydroxy-3,4-dihydro-2H- benzo[e][1,2]oxaborinine-8- carboxylic acid | 604.27 | 605 |
| 22 | 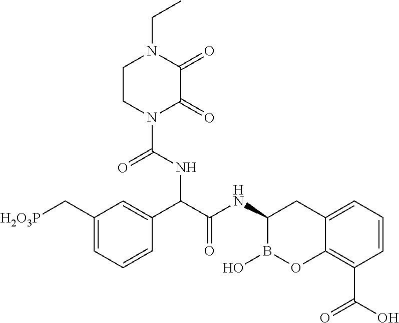 | (3R)-3-(2-(4-ethyl-2,3- dioxopiperazine-1-carboxamido)- 2-(3- (phosphonomethyl)phenyl) acetamido)-2- hydroxy-3,4-dihydro- 2H-benzo[e][1,2]oxaborinine-8- carboxylic acid | 602.30 | 603 |
| 23 | 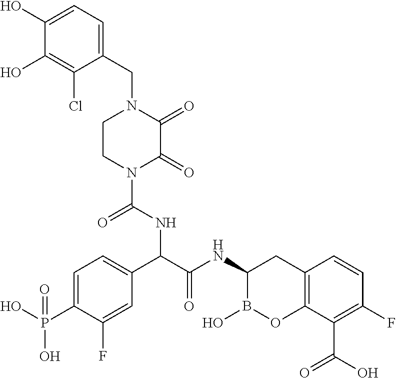 [SMILES: O=C(O)c1c(F)ccc2c1OB(O)[C@@H](NC(=O)C(NC(=O)N1CCN(Cc3ccc(O)c(O)c3Cl)C(=O)C1=O)c1ccc(P(=O)(O)O)c(F)c1)C2] | (3R)-3-(2-(4-(2-chloro-3,4- dihydroxybenzyl)-2,3- dioxopiperazine-1-carboxamido)- 2-(3-fluoro-4- phosphonophenyl)acetamido)-7- fluoro-2-hydroxy-3,4-dihydro- 2H-benzo[e][1,2]oxaborinine-8- carboxylic acid | 752.77 | 753 |
| 24 | 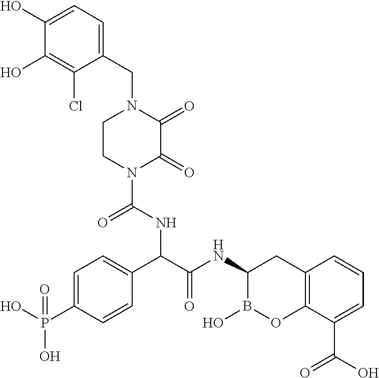 [SMILES: O=C(O)c1cccc2c1OB(O)[C@@H](NC(=O)C(NC(=O)N1CCN(Cc3ccc(O)c(O)c3Cl)C(=O)C1=O)c1ccc(P(=O)(O)O)cc1)C2] | (3R)-3-(2-(4-(2-chloro-3,4- dihydroxybenzyl)-2,3- dioxopiperazine-1-carboxamido)- 2-(4- phosphonophenyl)acetamido)-2- hydroxy-3,4-dihydro-2H- benzo[e][1,2]oxaborinine-8- carboxylic acid | 716.78 | 717 |
| 25 | 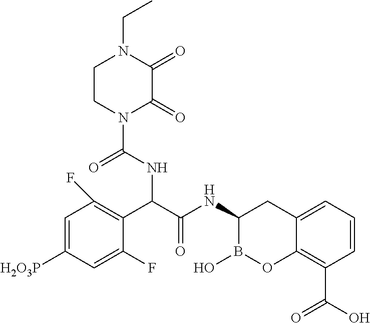 | (3R)-3-(2-(2,6-difluoro-4- phosphonophenyl)-2-(4-ethyl-2,3- dioxopiperazine-1- carboxamido)acetamido)-2- hydroxy-3,4-dihydro-2H- benzo[e][1,2]oxaborinine-8- carboxylic acid | 624.25 | 625 |
| 26 | 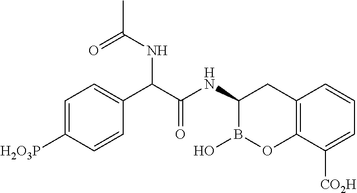 | (3R)-3-(2-acetamido-2-(4- phosphonophenyl)acetamido)-2- hydroxy-3,4-dihydro-2H- benzo[e][1,2]oxaborinine-8- carboxylic acid | 462.16 | 463 |
| 27 | 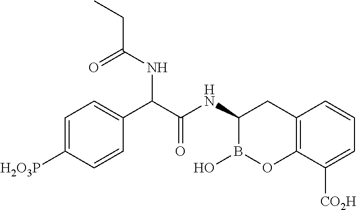 | (3R)-2-hydroxy-3-(2-(4- phosphonophenyl)-2- propionamidoacetamido)-3,4- dihydro-2H- benzo[e][1,2]oxaborinine-8- carboxylic acid | 476.19 | 477 |
| 28 | 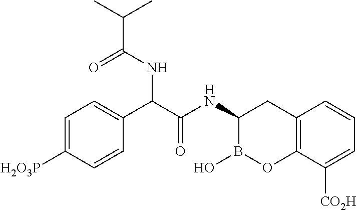 | (3R)-2-hydroxy-3-(2- isobutyramido-2-(4- phosphonophenyl)acetamido)- 3,4-dihydro-2H- benzo[e][1,2]oxaborinine-8- carboxylic acid | 490.21 | 491 |
| 29 | 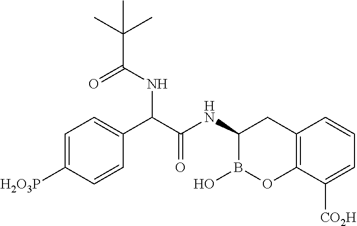 | (3R)-2-hydroxy-3-(2-(4- phosphonophenyl)-2- pivalamidoacetamido)-3,4- dihydro-2H- benzo[e][1,2]oxaborinine-8- carboxylic acid | 504.24 | 505 |
| 30 | 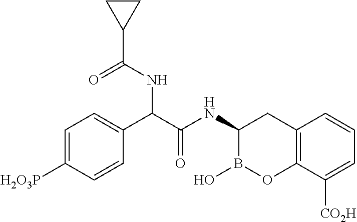 | (3R)-3-(2- (cyclopropanecarboxamido)-2-(4- phosphonophenyl)acetamido)-2- hydroxy-3,4-dihydro-2H- benzo[e][1,2]oxaborinine-8- carboxylic acid | 488.20 | 489 |
| 31 | 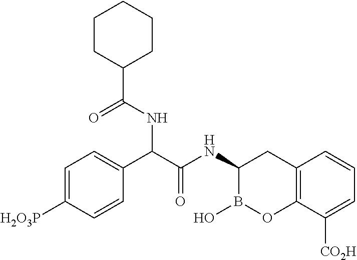 | (3R)-3-(2- (cyclohexanecarboxamido)-2-(4- phosphonophenyl)acetamido)-2- hydroxy-3,4-dihydro-2H- benzo[e][1,2]oxaborinine-8- carboxylic acid | 530.28 | 531 |
| 32 | 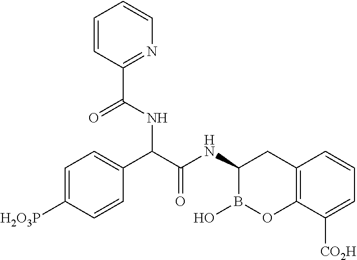 | (3R)-2-hydroxy-3-(2-(4- phosphonophenyl)-2- (picolinamido)acetamido)-3,4- dihydro-2H- benzo[e][1,2]oxaborinine-8- carboxylic acid | 525.22 | 526 |
| 33 | 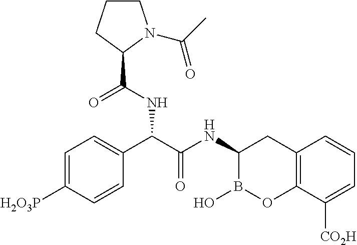 | (3R)-3-(2-((R)-1- acetylpyrrolidine-2- carboxamido)-2-(4- phosphonophenyl)acetamido)-2- hydroxy-3,4-dihydro-2H- benzo[e][1,2]oxaborinine-8- carboxylic acid | 559.28 | 560 |
| 34 | 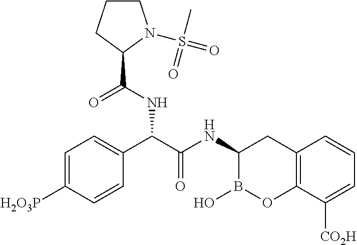 | (R)-2-hydroxy-3-((S)-2-((R)-1- (methylsulfonyl)pyrrolidine-2- carboxamido)-2-(4- phosphonophenyl)acetamido)- 3,4-dihydro-2H- benzo[e][1,2]oxaborinine-8- carboxylic acid | 595.32 | 596 |
| 35 | 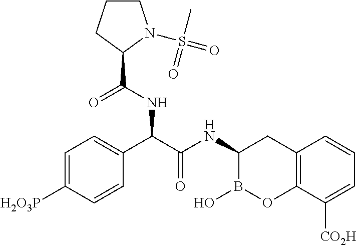 | (R)-2-hydroxy-3-((R)-2-((R)-1- (methylsulfonyl)pyrrolidine-2- carboxamido)-2-(4- phosphonophenyl)acetamido)- 3,4-dihydro-2H- benzo[e][1,2]oxaborinine-8- carboxylic acid | 595.32 | 596 |
| 36 | 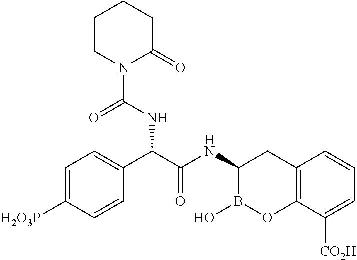 | (R)-2-hydroxy-3-((S)-2-(2- oxopiperidine-1-carboxamido)-2- (4-phosphonophenyl)acetamido)- 3,4-dihydro-2H- benzo[39e][1,2]oxaborinine-8- carboxylic acid | 545.25 | 546 |
| 37 | 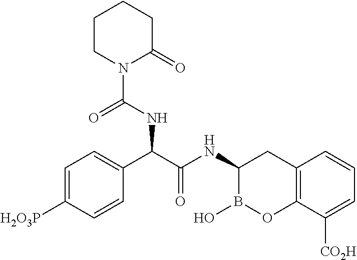 | (R)-2-hydroxy-3-((R)-2-(2- oxopiperidine-1-carboxamido)-2- (4-phosphonophenyl)acetamido)- 3,4-dihydro-2H- benzo[e][1,2]oxaborinine-8- carboxylic acid | 545.25 | 546 |
| 38 | 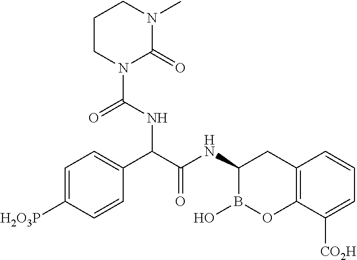 | (3R)-2-hydroxy-3-(2-(3-methyl- 2-oxohexahydropyrimidine-1- carboxamido)-2-(4- phosphonophenyl)acetamido)- 3,4-dihydro-2H- benzo[e][1,2]oxaborinine-8- carboxylic acid | 560.26 | 561 |
| 39 | 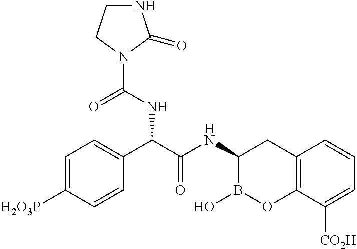 | (R)-2-hydroxy-3-((S)-2-(2- oxoimidazolidine-1- carboxamido)-2-(4- phosphonophenyl)acetamido)- 3,4-dihydro-2H- benzo[e][1,2]oxaborinine-8- carboxylic acid | 532.21 | 533 |
| 40 | 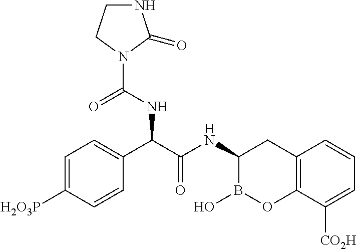 | (R)-2-hydroxy-3-((R)-2-(2- oxoimidazolidine-1- carboxamido)-2-(4- phosphonophenyl)acetamido)- 3,4-dihydro-2H- benzo[e][1,2]oxaborinine-8- carboxylic acid | 532.21 | 533 |
| 41 | 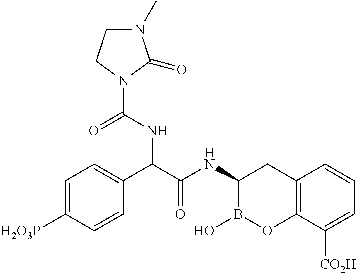 | (3R)-2-hydroxy-3-(2-(3-methyl- 2-oxoimidazolidine-1- carboxamido)-2-(4- phosphonophenyl)acetamido)- 3,4-dihydro-2H- benzo[e][1,2]oxaborinine-8- carboxylic acid | 546.24 | 547 |
| 42 | 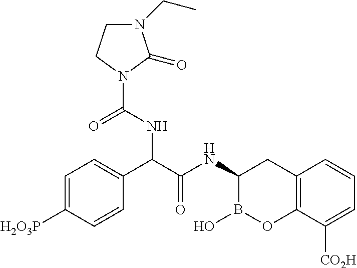 | (3R)-3-(2-(3-ethyl-2- oxoimidazolidine-1- carboxamido)-2-(4- phosphonophenyl)acetamido)-2- hydroxy-3,4-dihydro-2H- benzo[e][1,2]oxaborinine-8- carboxylic acid | 560.26 | 561 |
| 43 | 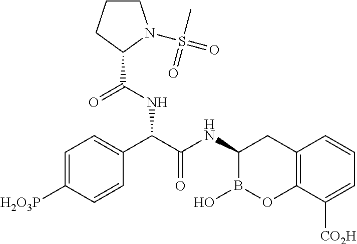 | (R)-2-hydroxy-3-((S)-2-((S)-1- (methylsulfonyl)pyrrolidine-2- carboxamido)-2-(4- phosphonophenyl)acetamido)- 3,4-dihydro-2H- benzo[e][1,2]oxaborinine-8- carboxylic acid | 595.32 | 596 |
| 44 | 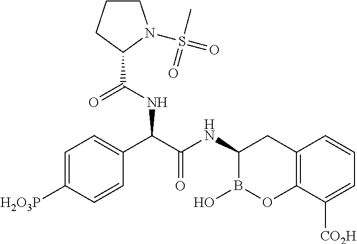 | (R)-2-hydroxy-3-((R)-2-((S)-1- (methylsulfonyl)pyrrolidine-2- carboxamido)-2-(4- phosphonophenyl)acetamido)- 3,4-dihydro-2H- benzo[e] [1,2]oxaborinine-8- carboxylic acid | 595.32 | 596 |
| 45 | 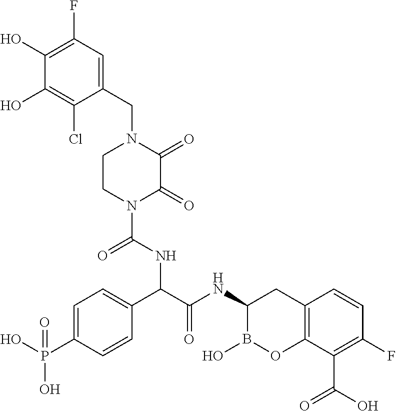 [SMILES: O=C(O)c1c(F)ccc2c1OB(O)[C@@H](NC(=O)C(NC(=O)N1CCN(Cc3cc(F)c(O)c(O)c3Cl)C(=O)C1=O)c1ccc(P(=O)(O)O)cc1)C2] | (3R)-3-(2-(4-(2-chloro-5-fluoro- 3,4-dihydroxybenzyl)-2,3- dioxopiperazine-1-carboxamido)- 2-(4- phosphonophenyl)acetamido)-7- fluoro-2-hydroxy-3,4-dihydro- 2H-benzo[e][1,2]oxaborinine-8- carboxylic acid | 752.77 | 753 |
| 46 | 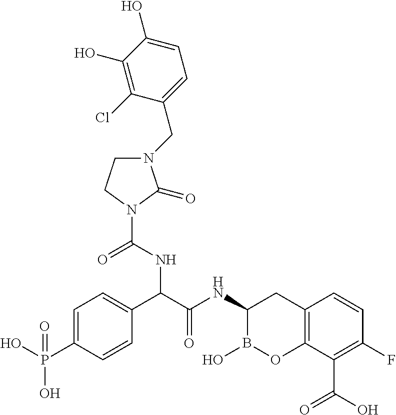 [SMILES: O=C(O)c1c(F)ccc2c1OB(O)[C@@H](NC(=O)C(NC(=O)N1CCN(Cc3ccc(O)c(O)c3Cl)C1=O)c1ccc(P(=O)(O)O)cc1)C2] | (3R)-3-(2-(3-(2-chloro-3,4- dihydroxybenzyl)-2- oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 706.76 | 707 |
| 47 | 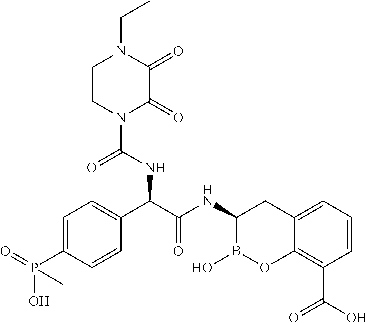 [SMILES: CCN1CCN(C(=O)N[C@@H](C(=O)N[C@H]2Cc3cccc(C(=O)O)c3OB2O)c2ccc(P(C)(=O)O)cc2)C(=O)C1=O] | (3R)-3-((2R)-2-(4-ethyl-2,3- dioxopiperazine-1-carboxamido)- 2-(4- (hydroxy(methyl)phosphoryl) phenyl)acetamido)-2- hydroxy-3,4- dihydro-2H- benzo[e][1,2]oxaborinine-8- carboxylic acid | 586.30 | 587 |
| 48 | 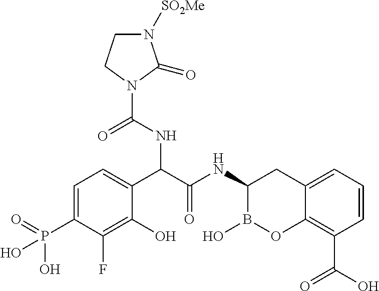 [SMILES: CS(=O)(=O)N1CCN(C(=O)NC(C(=O)N[C@H]2Cc3cccc(C(=O)O)c3OB2O)c2ccc(P(=O)(O)O)c(F)c2O)C1=O] | (3R)-3-(2-(3-fluoro-2-hydroxy-4- phosphonophenyl)-2-(3- (methylsulfonyl)-2- oxoimidazolidine-1- carboxamido)acetamido)-2- hydroxy-3,4-dihydro-2H- benzo[e][1,2]oxaborinine-8- carboxylic acid | 644.28 | 645 |
| 49 | 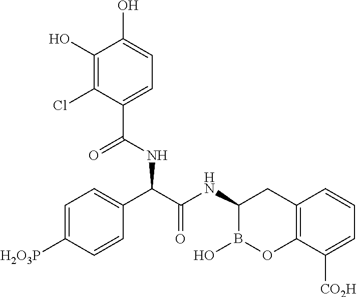 | (R)-3-((R)-2-(2-chloro-3,4- dihydroxybenzamido)-2-(4- phosphonophenyl)acetamido)-2- hydroxy-3,4-dihydro-2H- benzo[e][1,2]oxaborinine-8- carboxylic acid | 590.67 | 591 |
| 50 | 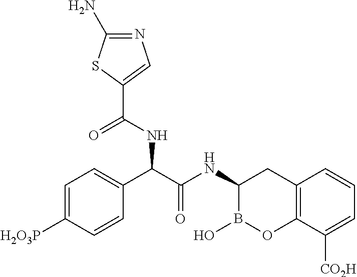 | (R)-3-((R)-2-(2-aminothiazole-5- carboxamido)-2-(4- phosphonophenyl)acetamido)-2- hydroxy-3,4-dihydro-2H- benzo[e][1,2]oxaborinine-8- carboxylic acid | 546.25 | 547 |
| 51 | 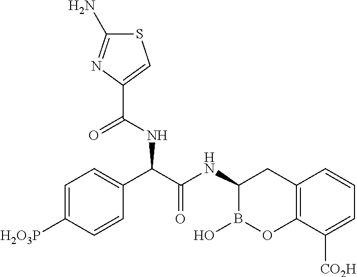 | (R)-3-((R)-2-(2-aminothiazole-4- carboxamido)-2-(4- phosphonophenyl)acetamido)-2- hydroxy-3,4-dihydro-2H- benzo[e][1,2]oxaborinine-8- carboxylic acid | 546.25 | 547 |
| 52 | 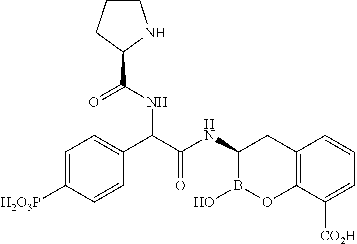 | (3R)-2-hydroxy-3-(2-(4- phosphonophenyl)-2-((R)- pyrrolidine-2- carboxamido)acetamido)-3,4- dihydro-2H- benzo[e][1,2]oxaborinine-8- carboxylic acid | 517.24 | 518 |
| 53 | 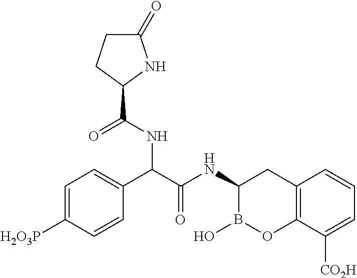 | (3R)-2-hydroxy-3-(2-((R)-5- oxopyrrolidine-2-carboxamido)- 2-(4- phosphonophenyl)acetamido)- 3,4-dihydro-2H- benzo[e][1,2]oxaborinine-8- carboxylic acid | 531.22 | 532 |
| 54 | 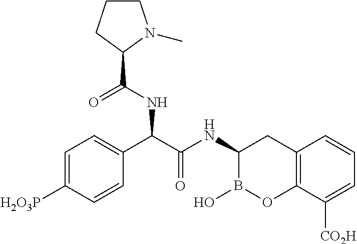 | (R)-2-hydroxy-3-((R)-2-((R)-1- methylpyrrolidine-2- carboxamido)-2-(4- phosphonophenyl)acetamido)- 3,4-dihydro-2H- benzo[e][1,2]oxaborinine-8- carboxylic acid | 531.26 | 532 |
| 56 | 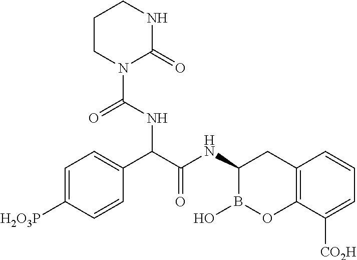 | (3R)-2-hydroxy-3-(2-(2- oxohexahydropyrimidine-1- carboxamido)-2-(4- phosphonophenyl)acetamido)- 3,4-dihydro-2H- benzo[e][1,2]oxaborinine-8- carboxylic acid | 546.24 | 547 |
| 57 | 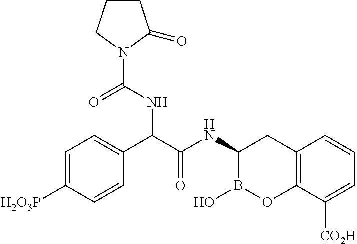 | (3R)-2-hydroxy-3-(2-(2- oxopyrrolidine-1-carboxamido)- 2-(4- phosphonophenyl)acetamido)- 3,4-dihydro-2H- benzo[e][1,2]oxaborinine-8- carboxylic acid | 531.22 | 532 |
| 58 | 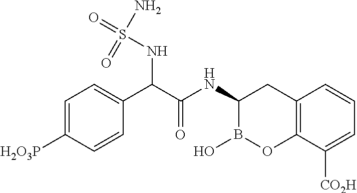 | (3R)-2-hydroxy-3-(2-(4- phosphonophenyl)-2- (sulfamoylamino)acetamido)-3,4- dihydro-2H- benzo[e][1,2]oxaborinine-8- carboxylic acid | 499.19 | 500 |
| 59 | 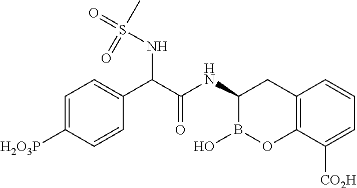 | (3R)-2-hydroxy-3-(2- (methylsulfonamido)-2-(4- phosphonophenyl)acetamido)- 3,4-dihydro-2H- benzo[e][1,2]oxaborinine-8- carboxylic acid | 498.21 | 499 |
| 60 | 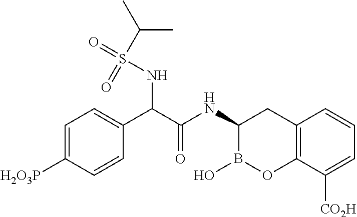 | (3R)-2-hydroxy-3-(2-((1- methylethyl)sulfonamido)-2-(4- phosphonophenyl)acetamido)- 3,4-dihydro-2H- benzo[e][1,2]oxaborinine-8- carboxylic acid | 526.26 | 527 |
| 61 | 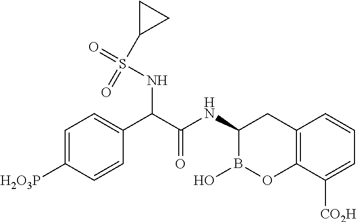 | (3R)-3-(2- (cyclopropanesulfonamido)-2-(4- phosphonophenyl)acetamido)-2- hydroxy-3,4-dihydro-2H- benzo[e][1,2]oxaborinine-8- carboxylic acid | 524.24 | 525 |
| 64 | 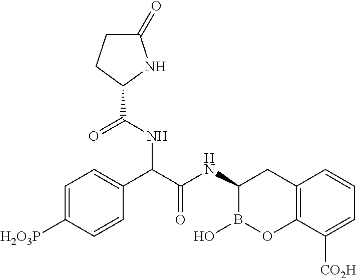 | (3R)-2-hydroxy-3-(2-((S)-5- oxopyrrolidine-2-carboxamido)- 2-(4- phosphonophenyl)acetamido)- 3,4-dihydro-2H- benzo[e][1,2]oxaborinine-8- carboxylic acid | 531.22 | 532 |
| 65 | 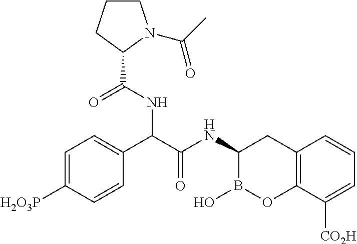 | (3R)-3-(2-((S)-1- acetylpyrrolidine-2- carboxamido)-2-(4- phosphonophenyl)acetamido)-2- hydroxy-3,4-dihydro-2H- benzo[e][1,2]oxaborinine-8- carboxylic acid | 559.27 | 560 |
| 66 | 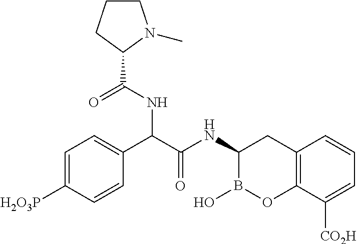 | (3R)-2-hydroxy-3-(2-((S)-1- methylpyrrolidine-2- carboxamido)-2-(4- phosphonophenyl)acetamido)- 3,4-dihydro-2H- benzo[e][1,2]oxaborinine-8- carboxylic acid | 531.26 | 532 |
| 120 | 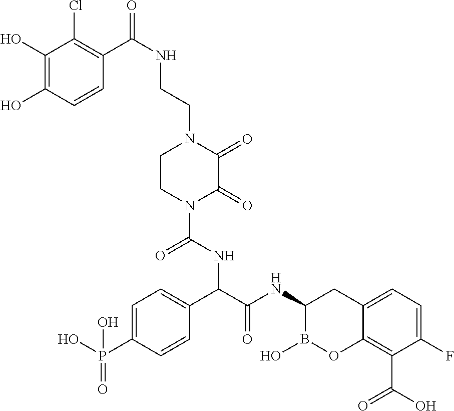 [SMILES: O=C(NCCN1CCN(C(=O)NC(C(=O)N[C@H]2Cc3ccc(F)c(C(=O)O)c3OB2O)c2ccc(P(=O)(O)O)cc2)C(=O)C1=O)c1ccc(O)c(O)c1Cl] | (3R)-3-(2-(4-(2-(2-chloro-3,4- dihydroxybenzamido)ethyl)-2,3- dioxopiperazine-1-carboxamido)- 2-(4- phosphonophenyl)acetamido)-7- fluoro-2-hydroxy-3,4-dihydro- 2H-benzo[e][1,2]oxaborinine-8- carboxylic acid | 791.83 | 791 |
| 160 | 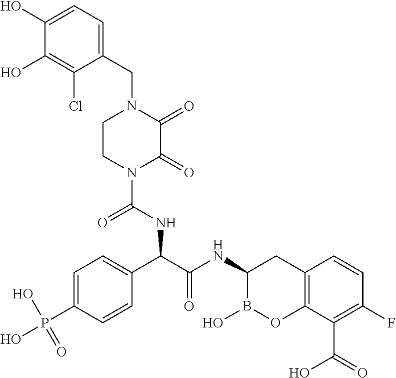 [SMILES: O=C(O)c1c(F)ccc2c1OB(O)[C@@H](NC(=O)[C@H](NC(=O)N1CCN(Cc3ccc(O)c(O)c3Cl)C(=O)C1=O)c1ccc(P(=O)(O)O)cc1)C2] | (R)-3-((R)-2-(4-(2-chloro-3,4- dihydroxybenzyl)-2,3- dioxopiperazine-1-carboxamido)- 2-(4- phosphonophenyl)acetamido)-7- fluoro-2-hydroxy-3,4-dihydro- 2H-benzo[e][1,2]oxaborinine-8- carboxylic acid | 734.77 | 735 |
| 161 | 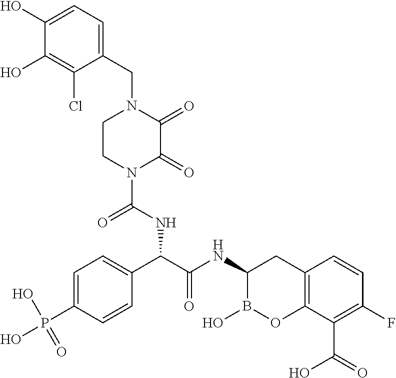 [SMILES: O=C(O)c1c(F)ccc2c1OB(O)[C@@H](NC(=O)[C@@H](NC(=O)N1CCN(Cc3ccc(O)c(O)c3Cl)C(=O)C1=O)c1ccc(P(=O)(O)O)cc1)C2] | (R)-3-((S)-2-(4-(2-chloro-3,4- dihydroxybenzyl)-2,3- dioxopiperazine-1-carboxamido)- 2-(4- phosphonophenyl)acetamido)-7- fluoro-2-hydroxy-3,4-dihydro- 2H-benzo[e][1,2]oxaborinine-8- carboxylic acid | 734.77 | 735 |
| 162 | 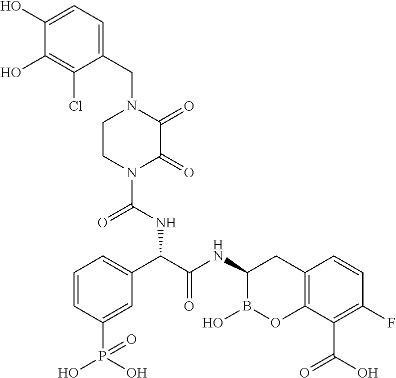 [SMILES: O=C(O)c1c(F)ccc2c1OB(O)[C@@H](NC(=O)[C@@H](NC(=O)N1CCN(Cc3ccc(O)c(O)c3Cl)C(=O)C1=O)c1cccc(P(=O)(O)O)c1)C2] | (R)-3-((S)-2-(4-(2-chloro-3,4- dihydroxybenzyl)-2,3- dioxopiperazine-1-carboxamido)- 2-(3- phosphonophenyl)acetamido)-7- fluoro-2-hydroxy-3,4-dihydro- 2H-benzo[e][1,2]oxaborinine-8- carboxylic acid | 734.77 | 734 |
| 163 | 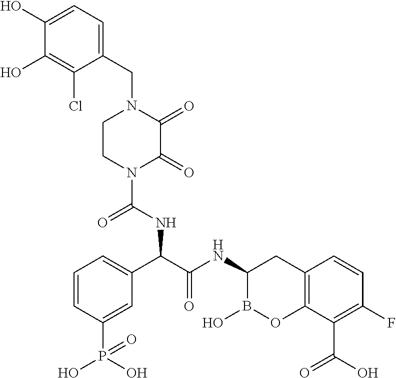 [SMILES: O=C(O)c1c(F)ccc2c1OB(O)[C@@H](NC(=O)[C@H](NC(=O)N1CCN(Cc3ccc(O)c(O)c3Cl)C(=O)C1=O)c1cccc(P(=O)(O)O)c1)C2] | (R)-3-((R)-2-(4-(2-chloro-3,4- dihydroxybenzyl)-2,3- dioxopiperazine-1-carboxamido)- 2-(3- phosphonophenyl)acetamido)-7- fluoro-2-hydroxy-3,4-dihydro- 2H-benzo[e][1,2]oxaborinine-8- carboxylic acid | 734.77 | 734 |
| 164 | 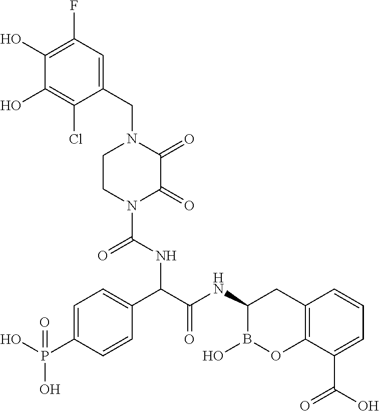 [SMILES: O=C(O)c1cccc2c1OB(O)[C@@H](NC(=O)C(NC(=O)N1CCN(Cc3cc(F)c(O)c(O)c3Cl)C(=O)C1=O)c1ccc(P(=O)(O)O)cc1)C2] | (3R)-3-(2-(4-(2-chloro-5-fluoro- 3,4-dihydroxybenzyl)-2,3- dioxopiperazine-1-carboxamido)- 2-(4- phosphonophenyl)acetamido)-2- hydroxy-3,4-dihydro-2H- benzo[e][1,2]oxaborinine-8- carboxylic acid | 734.77 | 734 |
| 165 | 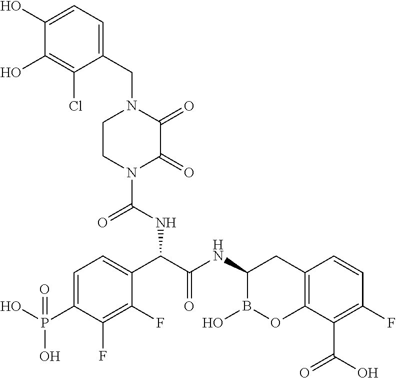 [SMILES: O=C(O)c1c(F)ccc2c1OB(O)[C@@H](NC(=O)[C@@H](NC(=O)N1CCN(Cc3ccc(O)c(O)c3Cl)C(=O)C1=O)c1ccc(P(=O)(O)O)c(F)c1F)C2] | (R)-3-((S)-2-(4-(2-chloro-3,4- dihydroxybenzyl)-2,3- dioxopiperazine-1-carboxamido)- 2-(2,3-difluoro-4- phosphonophenyl)acetamido)-7- fluoro-2-hydroxy-3,4-dihydro- 2H-benzo[e][1,2]oxaborinine-8- carboxylic acid | 770.76 | 770 |
| 166 | 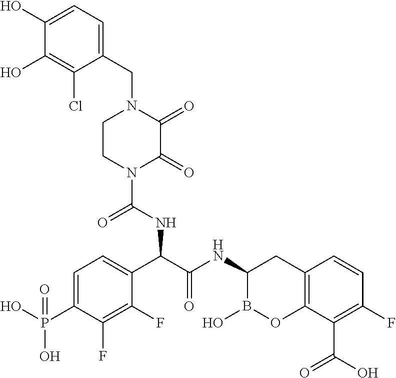 [SMILES: O=C(O)c1c(F)ccc2c1OB(O)[C@@H](NC(=O)[C@H](NC(=O)N1CCN(Cc3ccc(O)c(O)c3Cl)C(=O)C1=O)c1ccc(P(=O)(O)O)c(F)c1F)C2] | (R)-3-((R)-2-(4-(2-chloro-3,4- dihydroxybenzyl)-2,3- dioxopiperazine-1-carboxamido)- 2-(2,3-difluoro-4- phosphonophenyl)acetamido)-7- fluoro-2-hydroxy-3,4-dihydro- 2H-benzo[e][1,2]oxaborinine-8- carboxylic acid | 770.76 | 770 |
| 167 | 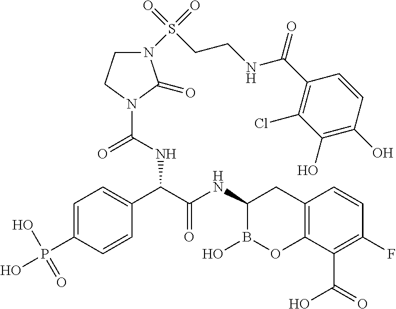 [SMILES: O=C(NCCS(=O)(=O)N1CCN(C(=O)N[C@H](C(=O)N[C@H]2Cc3ccc(F)c(C(=O)O)c3OB2O)c2ccc(P(=O)(O)O)cc2)C1=O)c1ccc(O)c(O)c1Cl] | (R)-3-((S)-2-(3-((2-(2-chloro-3,4- dihydroxybenzamido)ethyl) sulfonyl)-2- oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 827.87 | 828 |
| 168 | 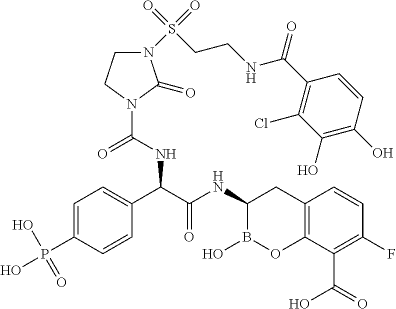 [SMILES: O=C(NCCS(=O)(=O)N1CCN(C(=O)N[C@@H](C(=O)N[C@H]2Cc3ccc(F)c(C(=O)O)c3OB2O)c2ccc(P(=O)(O)O)cc2)C1=O)c1ccc(O)c(O)c1Cl] | (R)-3-((R)-2-(3-((2-(2-chloro-3,4- dihydroxybenzamido)ethyl) sulfonyl)-2- oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 827.87 | 828 |
| 169 | 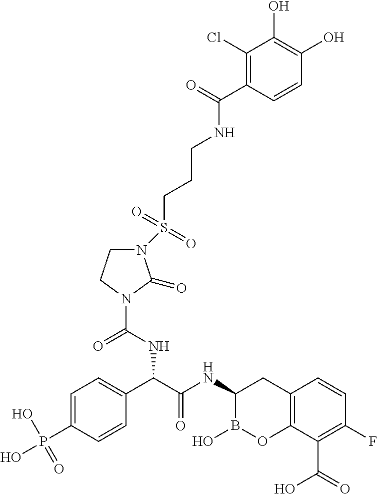 [SMILES: O=C(NCCCS(=O)(=O)N1CCN(C(=O)N[C@H](C(=O)N[C@H]2Cc3ccc(F)c(C(=O)O)c3OB2O)c2ccc(P(=O)(O)O)cc2)C1=O)c1ccc(O)c(O)c1Cl] | (R)-3-((S)-2-(3-((3-(2-chloro-3,4- dihydroxybenzamido)propyl) sulfonyl)-2- oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 841.90 | 828 |
| 170 | 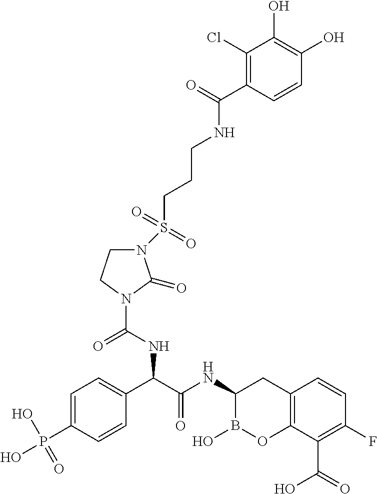 [SMILES: O=C(NCCCS(=O)(=O)N1CCN(C(=O)N[C@@H](C(=O)N[C@H]2Cc3ccc(F)c(C(=O)O)c3OB2O)c2ccc(P(=O)(O)O)cc2)C1=O)c1ccc(O)c(O)c1Cl] | (R)-3-((R)-2-(3-((3-(2-chloro-3,4- dihydroxybenzamido)propyl) sulfonyl)-2- oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 841.90 | 842 |
| 171 | 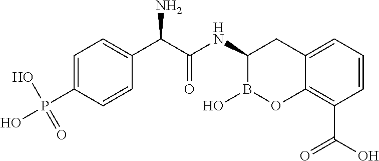 [SMILES: N[C@@H](C(=O)N[C@H]1Cc2cccc(C(=O)O)c2OB1O)c1ccc(P(=O)(O)O)cc1] | (R)-3-((R)-2-amino-2-(4- phosphonophenyl)acetamido)-2- hydroxy-3,4-dihydro-2H- benzo[e][1,2]oxaborinine-8- carboxylic acid | 420.12 | 421 |
| 172 | 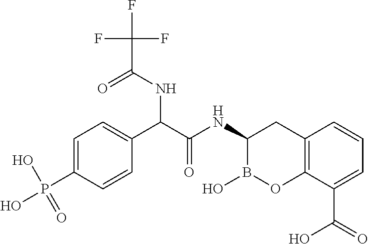 [SMILES: O=C(O)c1cccc2c1OB(O)[C@@H](NC(=O)C(NC(=O)C(F)(F)F)c1ccc(P(=O)(O)O)cc1)C2] | (3R)-2-hydroxy-3-(2-(4- phosphonophenyl)-2-(2,2,2- trifluoroacetamido)acetamido)- 3,4-dihydro-2H- benzo[e][1,2]oxaborinine-8- carboxylic acid | 516.13 | 517 |
| 173 | 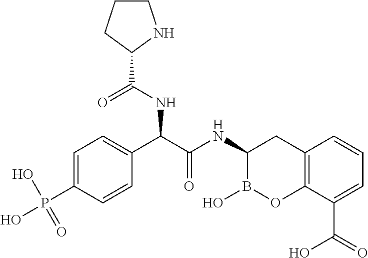 [SMILES: O=C(O)c1cccc2c1OB(O)[C@@H](NC(=O)[C@H](NC(=O)[C@@H]1CCCN1)c1ccc(P(=O)(O)O)cc1)C2] | (R)-2-hydroxy-3-((R)-2-(4- phosphonophenyl)-2-((S)- pyrrolidine-2- carboxamido)acetamido)-3,4- dihydro-2H- benzo[e][1,2]oxaborinine-8- carboxylic acid | 517.24 | 518 |
| 174 | 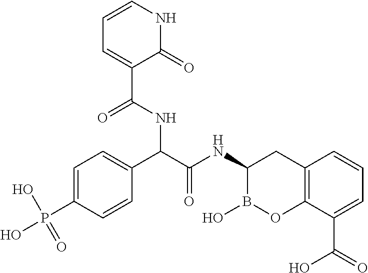 [SMILES: O=C(O)c1cccc2c1OB(O)[C@@H](NC(=O)C(NC(=O)c1ccc[nH]c1=O)c1ccc(P(=O)(O)O)cc1)C2] | (3R)-2-hydroxy-3-(2-(2-oxo-1,2- dihydropyridine-3-carboxamido)- 2-(4- phosphonophenyl)acetamido)- 3,4-dihydro-2H- benzo[e][1,2]oxaborinine-8- carboxylic acid | 541.22 | 542 |
| 175 | 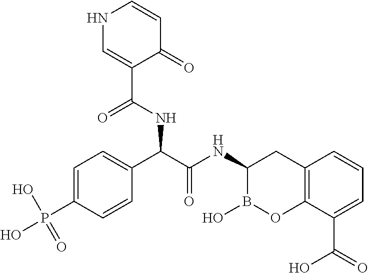 [SMILES: O=C(O)c1cccc2c1OB(O)[C@@H](NC(=O)[C@H](NC(=O)c1c[nH]ccc1=O)c1ccc(P(=O)(O)O)cc1)C2] | (R)-2-hydroxy-3-((R)-2-(4-oxo- 1,4-dihydropyridine-3- carboxamido)-2-(4- phosphonophenyl)acetamido)- 3,4-dihydro-2H- benzo[e][1,2]oxaborinine-8- carboxylic acid | 541.22 | 542 |
| 176 | 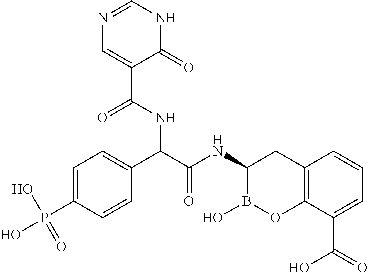 [SMILES: O=C(O)c1cccc2c1OB(O)[C@@H](NC(=O)C(NC(=O)c1cnc[nH]c1=O)c1ccc(P(=O)(O)O)cc1)C2] | (3R)-2-hydroxy-3-(2-(6-oxo-1,6- dihydropyrimidine-5- carboxamido)-2-(4- phosphonophenyl)acetamido)- 3,4-dihydro-2H- benzo[e][1,2]oxaborinine-8- carboxylic acid | 542.20 | 543 |
| 177 | 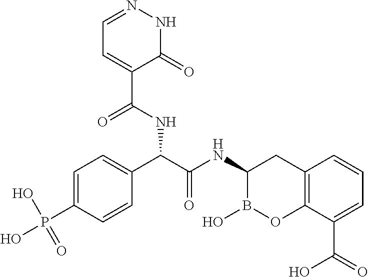 [SMILES: O=C(O)c1cccc2c1OB(O)[C@@H](NC(=O)[C@@H](NC(=O)c1ccn[nH]c1=O)c1ccc(P(=O)(O)O)cc1)C2] | (R)-2-hydroxy-3-((S)-2-(3-oxo- 2,3-dihydropyridazine-4- carboxamido)-2-(4- phosphonophenyl)acetamido)- 3,4-dihydro-2H- benzo[e][1,2]oxaborinine-8- carboxylic acid | 542.20 | 543 |
| 178 | 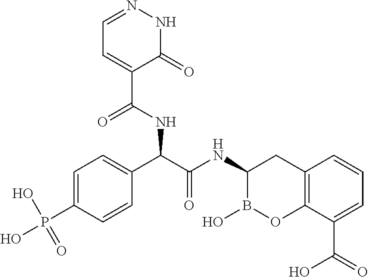 [SMILES: O=C(O)c1cccc2c1OB(O)[C@@H](NC(=O)[C@H](NC(=O)c1ccn[nH]c1=O)c1ccc(P(=O)(O)O)cc1)C2] | (R)-2-hydroxy-3-((R)-2-(3-oxo- 2,3-dihydropyridazine-4- carboxamido)-2-(4- phosphonophenyl)acetamido)- 3,4-dihydro-2H- benzo[e][1,2]oxaborinine-8- carboxylic acid | 542.20 | 543 |
| 179 | 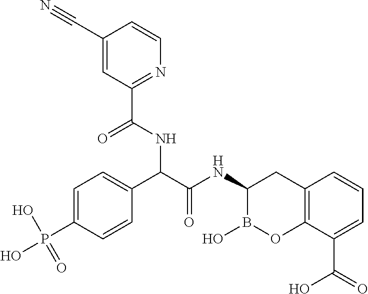 [SMILES: N#Cc1ccnc(C(=O)NC(C(=O)N[C@H]2Cc3cccc(C(=O)O)c3OB2O)c2ccc(P(=O)(O)O)cc2)c1] | (3R)-3-(2-(4-cyanopicolinamido)- 2-(4- phosphonophenyl)acetamido)-2- hydroxy-3,4-dihydro-2H- benzo[e][1,2]oxaborinine-8- carboxylic acid | 550.23 | 551 |
| 180 | 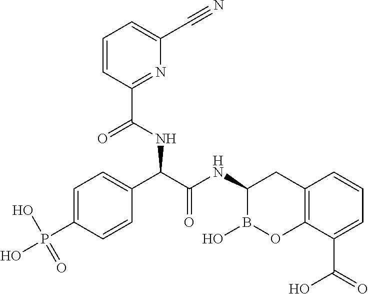 [SMILES: N#Cc1cccc(C(=O)N[C@@H](C(=O)N[C@H]2Cc3cccc(C(=O)O)c3OB2O)c2ccc(P(=O)(O)O)cc2)n1] | (R)-3-((R)-2-(6- cyanopicolinamido)-2-(4- phosphonophenyl)acetamido)-2- hydroxy-3,4-dihydro-2H- benzo[e][1,2]oxaborinine-8- carboxylic acid | 550.23 | 551 |
| 181 | 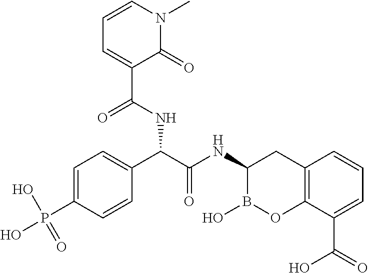 [SMILES: Cn1cccc(C(=O)N[C@H](C(=O)N[C@H]2Cc3cccc(C(=O)O)c3OB2O)c2ccc(P(=O)(O)O)cc2)c1=O] | (R)-2-hydroxy-3-((S)-2-(1- methyl-2-oxo-1,2- dihydropyridine-3-carboxamido)- 2-(4- phosphonophenyl)acetamido)- 3,4-dihydro-2H- benzo[e][1,2]oxaborinine-8- carboxylic acid | 555.24 | 556 |
| 182 | 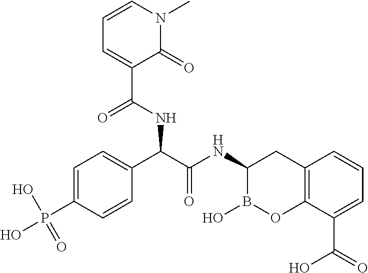 [SMILES: Cn1cccc(C(=O)N[C@@H](C(=O)N[C@H]2Cc3cccc(C(=O)O)c3OB2O)c2ccc(P(=O)(O)O)cc2)c1=O] | (R)-2-hydroxy-3-((R)-2-(1- methyl-2-oxo-1,2- dihydropyridine-3-carboxamido)- 2-(4- phosphonophenyl)acetamido)- 3,4-dihydro-2H- benzo[e][1,2]oxaborinine-8- carboxylic acid | 555.24 | 556 |
| 183 | 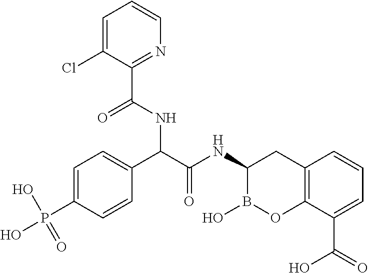 [SMILES: O=C(O)c1cccc2c1OB(O)[C@@H](NC(=O)C(NC(=O)c1ncccc1Cl)c1ccc(P(=O)(O)O)cc1)C2] | (3R)-3-(2-(3- chloropicolinamido)-2-(4- phosphonophenyl)acetamido)-2- hydroxy-3,4-dihydro-2H- benzo[e][1,2]oxaborinine-8- carboxylic acid | 559.66 | 560 |
| 184 | 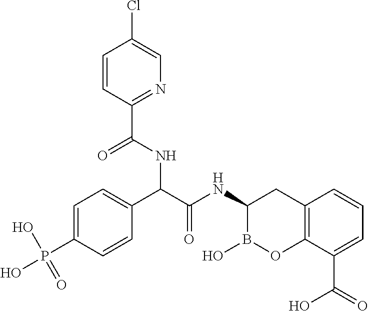 [SMILES: O=C(NC(C(=O)N[C@H]1Cc2cccc(C(=O)O)c2OB1O)c1ccc(P(=O)(O)O)cc1)c1ccc(Cl)cn1] | (3R)-3-(2-(5- chloropicolinamido)-2-(4- phosphonophenyl)acetamido)-2- hydroxy-3,4-dihydro-2H- benzo[e][1,2]oxaborinine-8- carboxylic acid | 559.66 | 560 |
| 186 | 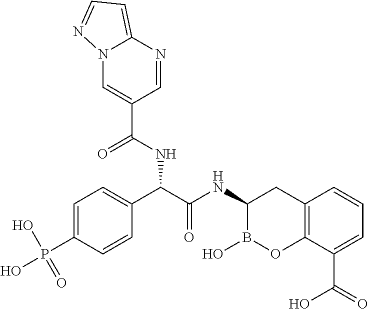 [SMILES: O=C(N[C@H](C(=O)N[C@H]1Cc2cccc(C(=O)O)c2OB1O)c1ccc(P(=O)(O)O)cc1)c1cnc2ccnn2c1] | (R)-2-hydroxy-3-((S)-2-(4- phosphonophenyl)-2- (pyrazolo[1,5-a]pyrimidine-6- carboxamido)acetamido)-3,4- dihydro-2H- benzo[e][1,2]oxaborinine-8- carboxylic acid | 565.24 | 566 |
| 187 | 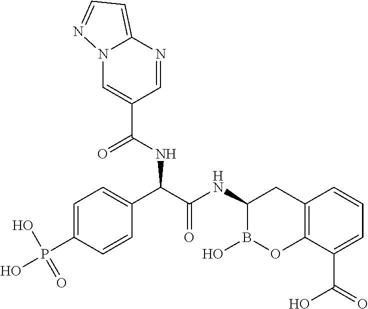 [SMILES: O=C(N[C@@H](C(=O)N[C@H]1Cc2cccc(C(=O)O)c2OB1O)c1ccc(P(=O)(O)O)cc1)c1cnc2ccnn2c1] | (R)-2-hydroxy-3-((R)-2-(4- phosphonophenyl)-2- (pyrazolo[1,5-a]pyrimidine-6- carboxamido)acetamido)-3,4- dihydro-2H- benzo[e][1,2]oxaborinine-8- carboxylic acid | 565.24 | 566 |
| 188 | 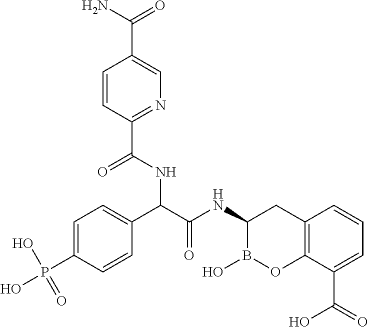 [SMILES: NC(=O)c1ccc(C(=O)NC(C(=O)N[C@H]2Cc3cccc(C(=O)O)c3OB2O)c2ccc(P(=O)(O)O)cc2)nc1] | (3R)-3-(2-(5- carbamoylpicolinamido)-2-(4- phosphonophenyl)acetamido)-2- hydroxy-3,4-dihydro-2H- benzo[e][1,2]oxaborinine-8- carboxylic acid | 568.24 | 569 |
| 189 | 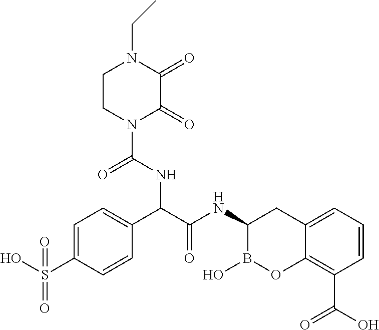 [SMILES: CCN1CCN(C(=O)NC(C(=O)N[C@H]2Cc3cccc(C(=O)O)c3OB2O)c2ccc(S(=O)(=O)O)cc2)C(=O)C1=O] | (3R)-3-(2-(4-ethyl-2,3- dioxopiperazine-1-carboxamido)- 2-(4-sulfophenyl)acetamido)-2- hydroxy-3,4-dihydro-2H- benzo[e][1,2]oxaborinine-8- carboxylic acid | 588.35 | 589 |
| 190 | 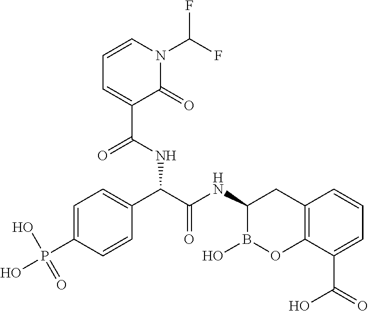 [SMILES: O=C(O)c1cccc2c1OB(O)[C@@H](NC(=O)[C@@H](NC(=O)c1cccn(C(F)F)c1=O)c1ccc(P(=O)(O)O)cc1)C2] | (R)-3-((S)-2-(1-(difluoromethyl)- 2-oxo-1,2-dihydropyridine-3- carboxamido)-2-(4- phosphonophenyl)acetamido)-2- hydroxy-3,4-dihydro-2H- benzo[e][1,2]oxaborinine-8- carboxylic acid | 591.22 | 592 |
| 191 | 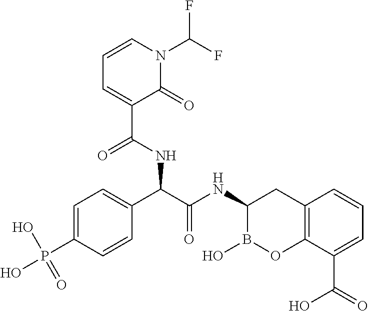 [SMILES: O=C(O)c1cccc2c1OB(O)[C@@H](NC(=O)[C@H](NC(=O)c1cccn(C(F)F)c1=O)c1ccc(P(=O)(O)O)cc1)C2] | (R)-3-((R)-2-(1-(difluoromethyl)- 2-oxo-1,2-dihydropyridine-3- carboxamido)-2-(4- phosphonophenyl)acetamido)-2- hydroxy-3,4-dihydro-2H- benzo[e][1,2]oxaborinine-8- carboxylic acid | 591.22 | 592 |
| 192 | 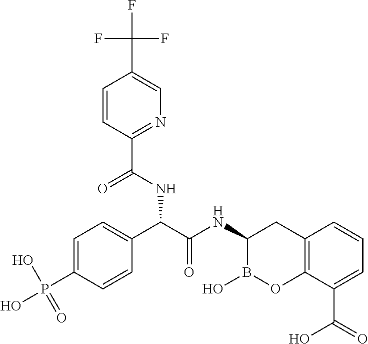 [SMILES: O=C(N[C@H](C(=O)N[C@H]1Cc2cccc(C(=O)O)c2OB1O)c1ccc(P(=O)(O)O)cc1)c1ccc(C(F)(F)F)cn1] | (R)-2-hydroxy-3-((S)-2-(4- phosphonophenyl)-2-(5- (trifluoromethyl)picolinamido) acetamido)-3,4-dihydro-2H- benzo[e][1,2]oxaborinine-8- carboxylic acid | 593.22 | 594 |
| 193 | 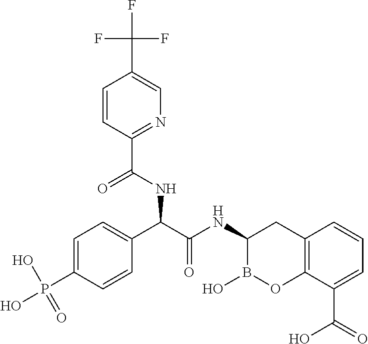 [SMILES: O=C(N[C@@H](C(=O)N[C@H]1Cc2cccc(C(=O)O)c2OB1O)c1ccc(P(=O)(O)O)cc1)c1ccc(C(F)(F)F)cn1] | (R)-2-hydroxy-3-((R)-2-(4- phosphonophenyl)-2-(5- (trifluoromethyl)picolinamido) acetamido)-3,4-dihydro-2H- benzo[e][1,2]oxaborinine-8- carboxylic acid | 593.22 | 594 |
| 194 | 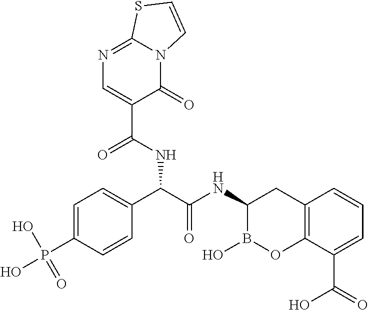 [SMILES: O=C(O)c1cccc2c1OB(O)[C@@H](NC(=O)[C@@H](NC(=O)c1cnc3sccn3c1=O)c1ccc(P(=O)(O)O)cc1)C2] | (R)-2-hydroxy-3-((S)-2-(5-oxo- 5H-thiazolo[3,2-a]pyrimidine-6- carboxamido)-2-(4- phosphonophenyl)acetamido)- 3,4-dihydro-2H- benzo[e][1,2]oxaborinine-8- carboxylic acid | 598.29 | 599 |
| 195 | 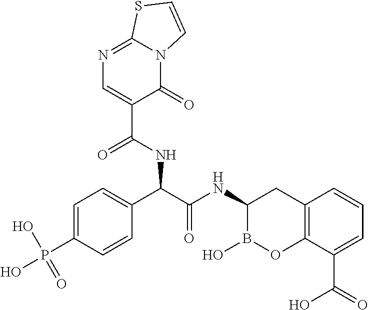 [SMILES: O=C(O)c1cccc2c1OB(O)[C@@H](NC(=O)[C@H](NC(=O)c1cnc3sccn3c1=O)c1ccc(P(=O)(O)O)cc1)C2] | (R)-2-hydroxy-3-((R)-2-(5-oxo- 5H-thiazolo[3,2-a]pyrimidine-6- carboxamido)-2-(4- phosphonophenyl)acetamido)- 3,4-dihydro-2H- benzo[e][1,2]oxaborinine-8- carboxylic acid | 598.29 | 599 |
| 196 | 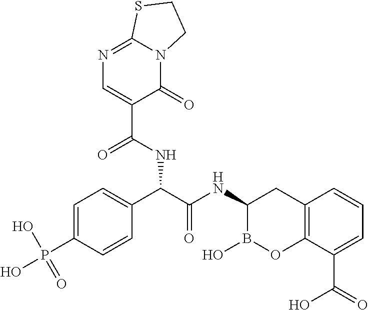 [SMILES: O=C(O)c1cccc2c1OB(O)[C@@H](NC(=O)[C@@H](NC(=O)c1cnc3n(c1=O)CCS3)c1ccc(P(=O)(O)O)cc1)C2] | (R)-2-hydroxy-3-((S)-2-(5-oxo- 2,3-dihydro-5H-thiazolo[3,2- alpyrimidine-6-carboxamido)-2- (4-phosphonophenyl)acetamido)- 3,4-dihydro-2H- benzo[e][1,2]oxaborinine-8- carboxylic acid | 600.30 | 601 |
| 197 | 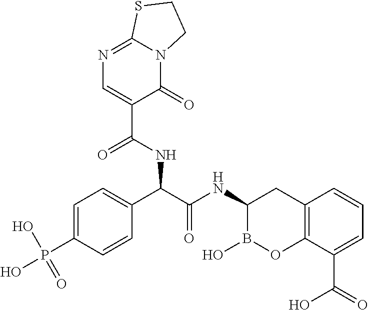 [SMILES: O=C(O)c1cccc2c1OB(O)[C@@H](NC(=O)[C@H](NC(=O)c1cnc3n(c1=O)CCS3)c1ccc(P(=O)(O)O)cc1)C2] | (R)-2-hydroxy-3-((R)-2-(5-oxo- 2,3-dihydro-5H-thiazolo[3,2- alpyrimidine-6-carboxamido)-2- (4-phosphonophenyl)acetamido)- 3,4-dihydro-2H- benzo[e][1,2]oxaborinine-8- carboxylic acid | 600.30 | 601 |
| 198 | 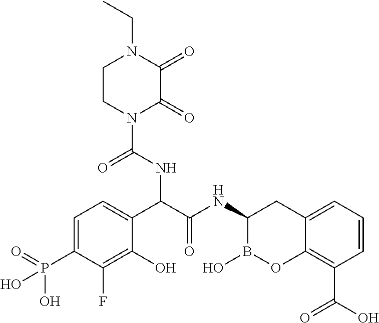 [SMILES: CCN1CCN(C(=O)NC(C(=O)N[C@H]2Cc3cccc(C(=O)O)c3OB2O)c2ccc(P(=O)(O)O)c(F)c2O)C(=O)C1=O] | (3R)-3-(2-(4-ethyl-2,3- dioxopiperazine-1-carboxamido)- 2-(3-fluoro-2-hydroxy-4- phosphonophenyl)acetamido)-2- hydroxy-3,4-dihydro-2H- benzo[e][1,2]oxaborinine-8- carboxylic acid | 622.26 | 623 |
| 494 | 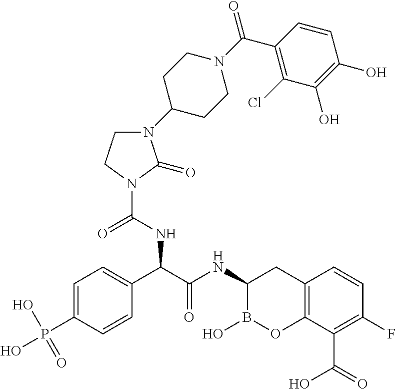 [SMILES: O=C(O)c1c(F)ccc2c1OB(O)[C@@H](NC(=O)[C@H](NC(=O)N1CCN(C3CCN(C(=O)c4ccc(O)c(O)c4Cl)CC3)C1=O)c1ccc(P(=O)(O)O)cc1)C2] | (R)-3-((R)-2-(3-(1-(2-chloro-3,4- dihydroxybenzoyl)piperidin-4- yl)-2-oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 803.88 | 804 |
| 495 | 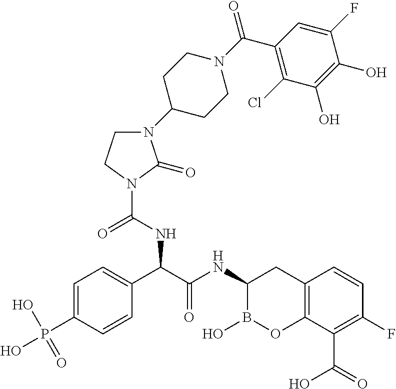 [SMILES: O=C(O)c1c(F)ccc2c1OB(O)[C@@H](NC(=O)[C@H](NC(=O)N1CCN(C3CCN(C(=O)c4cc(F)c(O)c(O)c4Cl)CC3)C1=O)c1ccc(P(=O)(O)O)cc1)C2] | (R)-3-((R)-2-(3-(1-(2-chloro-5- fluoro-3,4- dihydroxybenzoyl)piperidin-4- yl)-2-oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 821.87 | 822 |
| 496 | 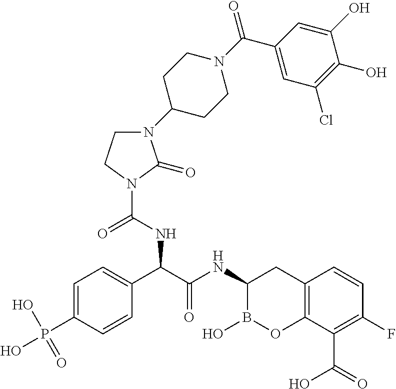 [SMILES: O=C(O)c1c(F)ccc2c1OB(O)[C@@H](NC(=O)[C@H](NC(=O)N1CCN(C3CCN(C(=O)c4cc(O)c(O)c(Cl)c4)CC3)C1=O)c1ccc(P(=O)(O)O)cc1)C2] | (R)-3-((R)-2-(3-(1-(3-chloro-4,5- dihydroxybenzoyl)piperidin-4- yl)-2-oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 803.88 | 804 |
| 497 | 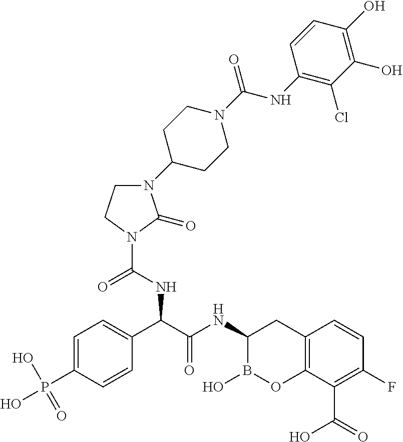 [SMILES: O=C(O)c1c(F)ccc2c1OB(O)[C@@H](NC(=O)[C@H](NC(=O)N1CCN(C3CCN(C(=O)Nc4ccc(O)c(O)c4Cl)CC3)C1=O)c1ccc(P(=O)(O)O)cc1)C2] | (R)-3-((R)-2-(3-(1-((2-chloro-3,4- dihydroxyphenyl)carbamoyl) piperidin-4-yl)- 2-oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 818.90 | 819 |
| 498 | 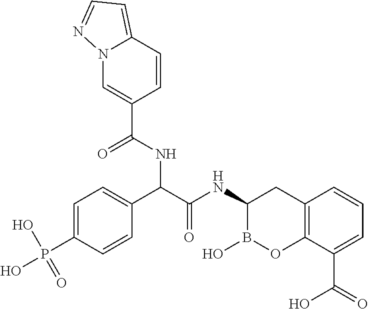 [SMILES: O=C(NC(C(=O)N[C@H]1Cc2cccc(C(=O)O)c2OB1O)c1ccc(P(=O)(O)O)cc1)c1ccc2ccnn2c1] | (3R)-2-hydroxy-3-(2-(4- phosphonophenyl)-2- (pyrazolo[1,5-a]pyridine-6- carboxamido)acetamido)-3,4- dihydro-2H- benzo[e][1,2]oxaborinine-8- carboxylic acid | 564.25 | 565 |
| 499 | 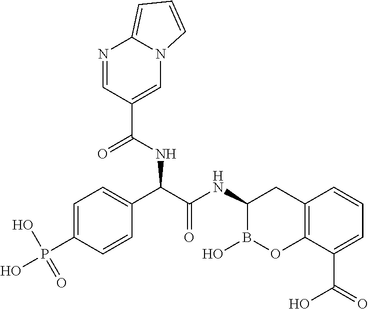 [SMILES: O=C(N[C@@H](C(=O)N[C@H]1Cc2cccc(C(=O)O)c2OB1O)c1ccc(P(=O)(O)O)cc1)c1cnc2cccn2c1] | (R)-2-hydroxy-3-((R)-2-(4- phosphonophenyl)-2- (pyrrolo[1,2-a]pyrimidine-3- carboxamido)acetamido)-3,4- dihydro-2H- benzo[e][1,2]oxaborinine-8- carboxylic acid | 564.25 | 565 |
| 500 | 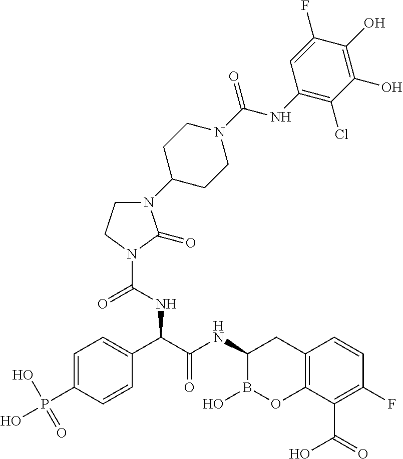 [SMILES: O=C(O)c1c(F)ccc2c1OB(O)[C@@H](NC(=O)[C@H](NC(=O)N1CCN(C3CCN(C(=O)Nc4cc(F)c(O)c(O)c4Cl)CC3)C1=O)c1ccc(P(=O)(O)O)cc1)C2] | (R)-3-((R)-2-(3-(1-((2-chloro-5- fluoro-3,4- dihydroxyphenyl)carbamoyl) piperidin-4-yl)- 2-oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 836.89 | 837 |
| 501 | 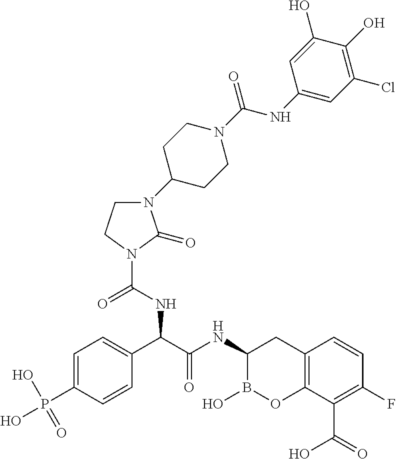 [SMILES: O=C(O)c1c(F)ccc2c1OB(O)[C@@H](NC(=O)[C@H](NC(=O)N1CCN(C3CCN(C(=O)Nc4cc(O)c(O)c(Cl)c4)CC3)C1=O)c1ccc(P(=O)(O)O)cc1)C2] | (R)-3-((R)-2-(3-(1-((3-chloro-4,5- dihydroxyphenyl)carbamoyl) piperidin-4-yl)-2- oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 818.90 | 819 |
| 502 | 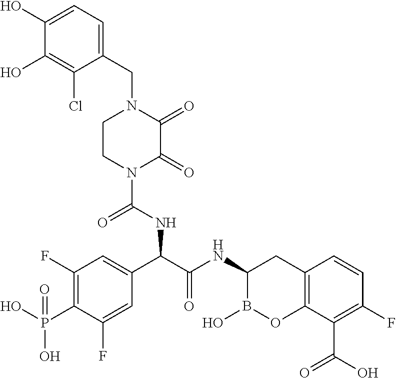 [SMILES: O=C(O)c1c(F)ccc2c1OB(O)[C@@H](NC(=O)[C@H](NC(=O)N1CCN(Cc3ccc(O)c(O)c3Cl)C(=O)C1=O)c1cc(F)c(P(=O)(O)O)c(F)c1)C2] | (R)-3-((R)-2-(4-(2-chloro-3,4- dihydroxybenzyl)-2,3- dioxopiperazine-1-carboxamido)- 2-(3,5-difluoro-4- phosphonophenyl)acetamido)-7- fluoro-2-hydroxy-3,4-dihydro- 2H-benzo[e][1,2]oxaborinine-8- carboxylic acid | 770.75 | 770 |
| 503 | 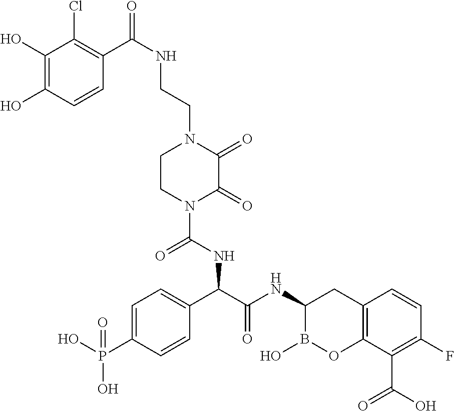 [SMILES: O=C(NCCN1CCN(C(=O)N[C@@H](C(=O)N[C@H]2Cc3ccc(F)c(C(=O)O)c3OB2O)c2ccc(P(=O)(O)O)cc2)C(=O)C1=O)c1ccc(O)c(O)c1Cl] | (R)-3-((R)-2-(4-(2-(2-chloro-3,4- dihydroxybenzamido)ethyl)-2,3- dioxopiperazine-1-carboxamido)- 2-(4- phosphonophenyl)acetamido)-7- fluoro-2-hydroxy-3,4-dihydro- 2H-benzo[e][1,2]oxaborinine-8- carboxylic acid | 791.83 | 792 |
| 504 | 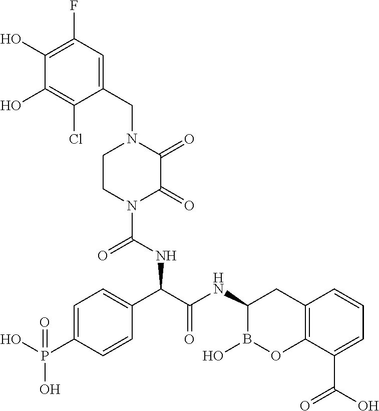 [SMILES: O=C(O)c1cccc2c1OB(O)[C@@H](NC(=O)[C@H](NC(=O)N1CCN(Cc3cc(F)c(O)c(O)c3Cl)C(=O)C1=O)c1ccc(P(=O)(O)O)cc1)C2] | (R)-3-((R)-2-(4-(2-chloro-5- fluoro-3,4-dihydroxybenzyl)-2,3- dioxopiperazine-1-carboxamido)- 2-(4- phosphonophenyl)acetamido)-2- hydroxy-3,4-dihydro-2H- benzo[e][1,2]oxaborinine-8- carboxylic acid | 791.83 | 735 |
| 505 | 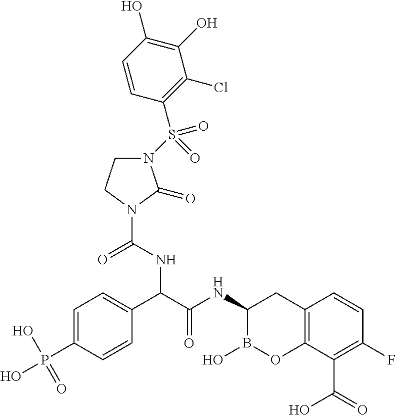 [SMILES: O=C(O)c1c(F)ccc2c1OB(O)[C@@H](NC(=O)C(NC(=O)N1CCN(S(=O)(=O)c3ccc(O)c(O)c3Cl)C1=O)c1ccc(P(=O)(O)O)cc1)C2] | (3R)-3-(2-(3-((2-chloro-3,4- dihydroxyphenyl)sulfonyl)-2- oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 791.83 | 757 |
| 506 | 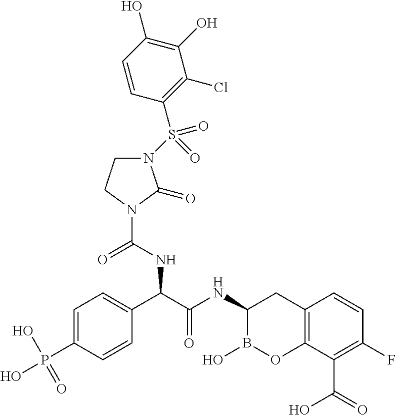 [SMILES: O=C(O)c1c(F)ccc2c1OB(O)[C@@H](NC(=O)[C@H](NC(=O)N1CCN(S(=O)(=O)c3ccc(O)c(O)c3Cl)C1=O)c1ccc(P(=O)(O)O)cc1)C2] | (R)-3-((R)-2-(3-((2-chloro-3,4- dihydroxyphenyl)sulfonyl)-2- oxoimidazolidine-1- carboxamido)-2-(4- phosphonophenyl)acetamido)-7- fluoro-2-hydroxy-3,4-dihydro- 2H-benzo[e][1,2]oxaborinine-8- carboxylic acid | 791.83 | 757 |
| 509 | 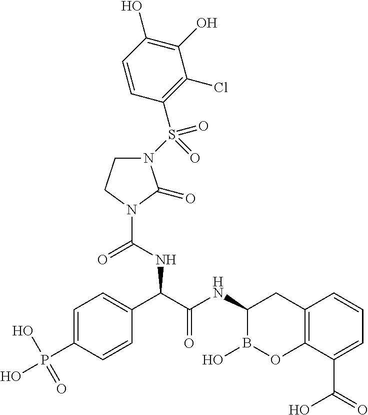 [SMILES: O=C(O)c1cccc2c1OB(O)[C@@H](NC(=O)[C@H](NC(=O)N1CCN(S(=O)(=O)c3ccc(O)c(O)c3Cl)C1=O)c1ccc(P(=O)(O)O)cc1)C2] | (R)-3-((R)-2-(3-((2-chloro-3,4- dihydroxyphenyl)sulfonyl)-2- oxoimidazolidine-1- carboxamido)-2-(4- phosphonophenyl)acetamido)-2- hydroxy-3,4-dihydro-2H- benzo[e][1,2]oxaborinine-8- carboxylic acid | 738.80 | 738 |

TABLE 2
Example compounds that may be prepared in analogy to the procedures of Examples 1-198.

| Ex. | Structure | Name | MW |
|---|---|---|---|
| 55 | 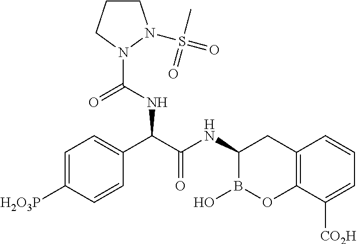 | (R)-2-hydroxy-3-((R)-2-(2- (methylsulfonyl)pyrazolidine-1-carboxamido)-2- (4-phosphonophenyl)acetamido)-3,4-dihydro-2H- benzo[e][1,2]oxaborinine-8-carboxylic acid | 596.31 |
| 62 | 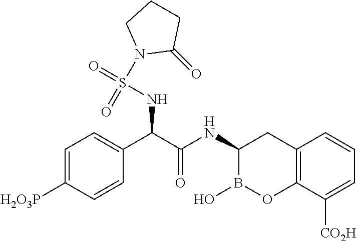 | (R)-2-hydroxy-3-((R)-2-((2-oxopyrrolidine)-1- sulfonamido)-2-(4-phosphonophenyl)acetamido)- 3,4-dihydro-2H-benzo[e][1,2]oxaborinine-8- carboxylic acid | 567.27 |
| 63 | 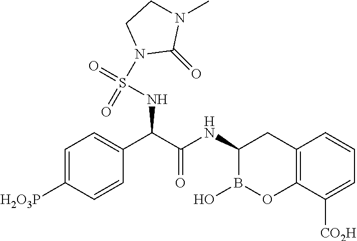 | (R)-2-hydroxy-3-((R)-2-((3-methyl-2- oxoimidazolidine)-1-sulfonamido)-2-(4- phosphonophenyl)acetamido)-3,4-dihydro-2H- benzo[e][1,2]oxaborinine-8-carboxylic acid | 582.28 |
| 67 | 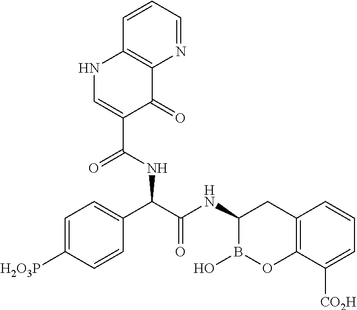 | (R)-2-hydroxy-3-((R)-2-(4-oxo-1,4-dihydro-1,5- naphthyridine-3-carboxamido)-2-(4- phosphonophenyl)acetamido)-3,4-dihydro-2H- benzo[e][1,2]oxaborinine-8-carboxylic acid | 592.26 |
| 68 | 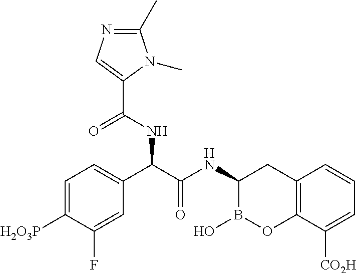 | (R)-3-((R)-2-(1,2-dimethyl-1H-imidazole-5- carboxamido)-2-(3-fluoro-4- phosphonophenyl)acetamido)-2-hydroxy-3,4- dihydro-2H-benzo[e][1,2]oxaborinine-8- carboxylic acid | 560.24 |
| 69 | 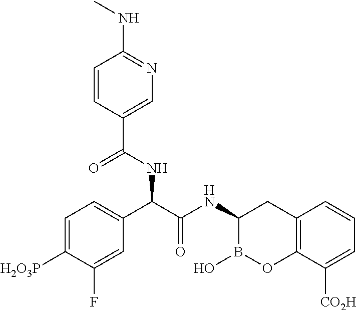 | (R)-3-((R)-2-(3-fluoro-4-phosphonophenyl)-2-(6- (methylamino)nictotinamido)acetamido)-2- hydroxy-3,4-dihydro-2H-benzo [e][1,2]oxaborinine-8-carboxylic acid | 572.25 |
| 70 | 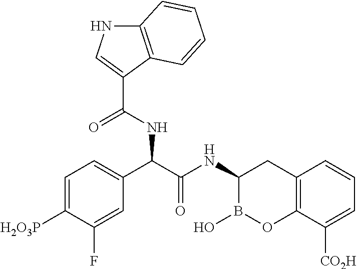 | (R)-3-((R)-2-(3-fluoro-4-phosphonophenyl)-2- (1H-indole-3-carboxamido)acetamido)-2- hydroxy-3,4-dihydro-2H- benzo[e][1,2]oxaborinine-8-carboxylic acid | 581.26 |
| 71 | 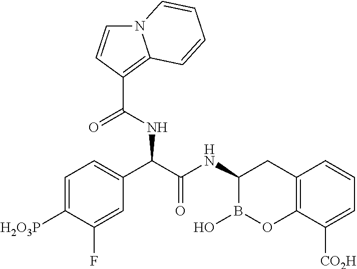 | (R)-3-((R)-2-(3-fluoro-4-phosphonophenyl)-2- (indolizine-1-carboxamido)acetamido)-2- hydroxy-3,4-dihydro-2H- benzo[e][1,2]oxaborinine-8-carboxylic acid | 581.26 |
| 72 | 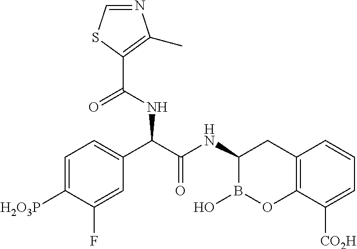 | (R)-3-((R)-2-(3-fluoro-4-phosphonophenyl)-2-(4- methylthiazole-5-carboxamido)acetamido)-2- hydroxy-3,4-dihydro-2H- benzo[e][1,2]oxaborinine-8-carboxylic acid | 563.26 |
| 73 | 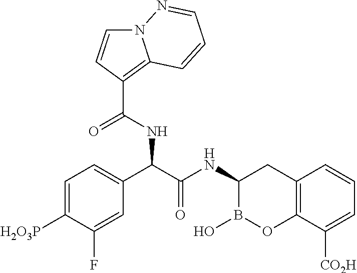 | (R)-3-((R)-2-(3-fluoro-4-phosphonophenyl)-2- (pyrrolo[1,2-b]pyridazine-5- carboxamido)acetamido)-2-hydroxy-3,4-dihydro- 2H-benzo[e][1,2]oxaborinine-8-carboxylic acid | 582.24 |
| 74 | 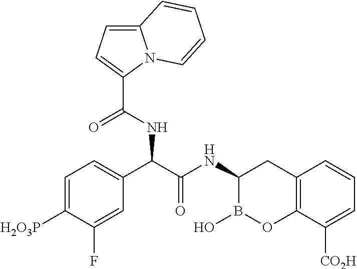 | (R)-3-((R)-2-(3-fluoro-4-phosphonophenyl)-2- (indolizine-3-carboxamido)acetamido)-2- hydroxy-3,4-dihydro-2H- benzo[e][1,2]oxaborinine-8-carboxylic acid | 581.26 |
| 75 | 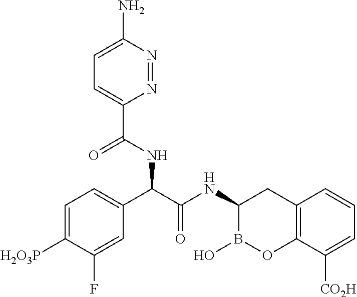 | (R)-3-((R)-2-(6-aminopyridazine-3- carboxamido)-2-(3-fluoro-4- phosphonophenyl)acetamido)-2-hydroxy-3,4- dihydro-2H- benzo[e][1,2]oxaborinine-8-carboxylic acid | 559.21 |
| 76 | 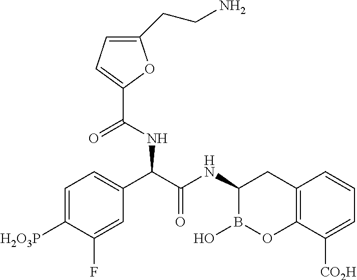 | (R)-3-((R)-2-(5-(2-aminooethyl)furan-2- carboxamido)-2-(3-fluoro-4- phosphonophenyl)acetamido)-2-hydroxy-3,4- dihydro-2H- benzo[e][1,2]oxaborinine-8-carboxylic acid | 575.25 |
| 77 | 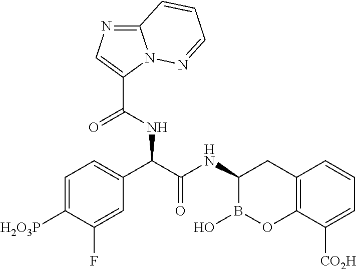 | (R)-3-((R)-2-(3-fluoro-4-phosphonophenyl)-2- (imidazo[1,2-b]pyridazine-3- carboxamido)acetamido)-2-hydroxy-3,4-dihydro- 2H-benzo[e][1,2]oxaborinine-8-carboxylic acid | 583.23 |
| 78 | 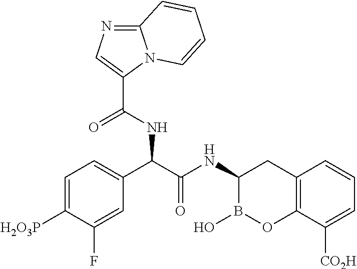 | (R)-3-((R)-2-(3-fluoro-4-phosphonophenyl)-2- (imidazo[1,2-a]pyridine-3- carboxamido)acetamido)-2-hydroxy-3,4-dihydro- 2H-benzo[e][1,2]oxaborinine-8-carboxylic acid | 582.24 |
| 79 | 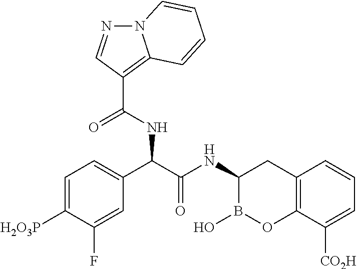 | (R)-3-((R)-2-(3-fluoro-4-phosphonophenyl)-2- (pyrazolo[1,5-a]pyridine-3- carboxamido)acetamido)-2-hydroxy-3,4-dihydro- 2H-benzo[e][1,2]oxaborinine-8-carboxylic acid | 582.24 |
| 80 | 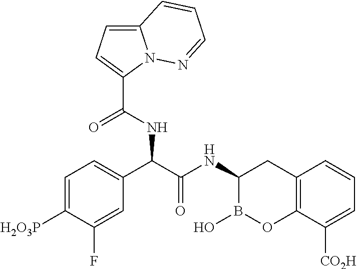 | (R)-3-((R)-2-(3-fluoro-4-phosphonophenyl)-2- (pyrrolo[1,2-b]pyridazine-7- carboxamido)acetamido)-2-hydroxy-3,4-dihydro- 2H-benzo[e][1,2]oxaborinine-8-carboxylic acid | 582.24 |
| 81 | 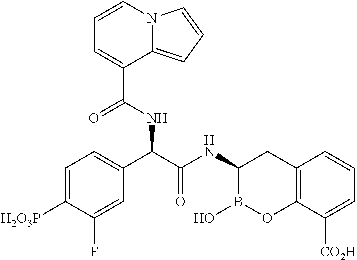 | (R)-3-((R)-2-(3-fluoro-4-phosphonophenyl)-2- (indolizine-8-carboxamido)acetamido)-2- hydroxy-3,4-dihydro-2H- benzo[e][1,2]oxaborinine-8-carboxylic acid | 581.26 |
| 82 | 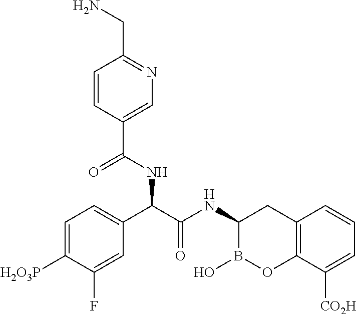 | (R)-3-((R)-2-(6-(aminomethyl)nicotinamido)-2- (3-fluoro-4-phosphonophenyl)acetamido)-2- hydroxy-3,4-dihydro-2H- benzo[e][1,2]oxaborinine-8-carboxylic acid | 572.25 |
| 83 | 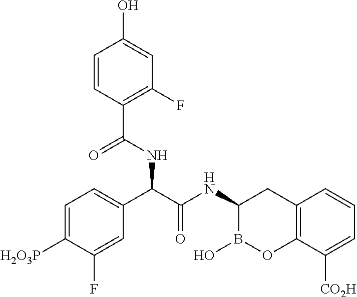 | (R)-3-((R)-2-(2-fluoro-4-hydroxybenzamido)-2- (3-fluoro-4-phosphonophenyl)acetamido)-2- hydroxy-3,4-dihydro-2H- benzo[e][1,2]oxaborinine-8-carboxylic acid | 576.21 |
| 84 | 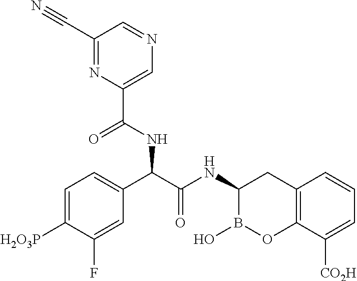 | (R)-3-((R)-2-(6-cyanopyrazine-2-carboxamido)- 2-(3-fluoro-4-phosphonophenyl)acetamido)-2- hydroxy-3,4-dihydro-2H- benzo[e][1,2]oxaborinine-8-carboxylic acid | 569.21 |
| 85 | 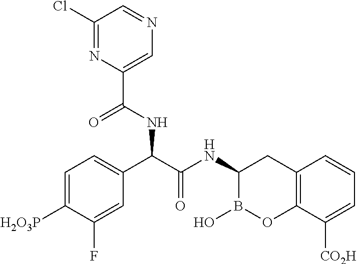 | (R)-3-((R)-2-(6-chloropyrazine-2-carboxamido)- 2-(3-fluoro-4-phosphonophenyl)acetamido)-2- hydroxy-3,4-dihydro-2H- benzo[e][1,2]oxaborinine-8-carboxylic acid | 578.64 |
| 86 | 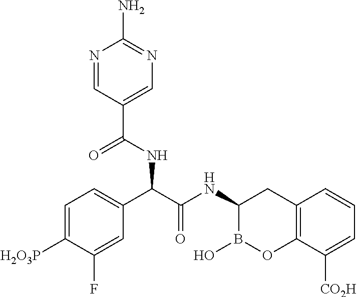 | (R)-3-((R)-2-(2-aminopyrimidine-5- carboxamido)-2-(3-fluoro-4- phosphonophenyl)acetamido)-2-hydroxy-3,4- dihydro-2H- benzo[e][1,2]oxaborinine-8-carboxylic acid | 559.21 |
| 87 | 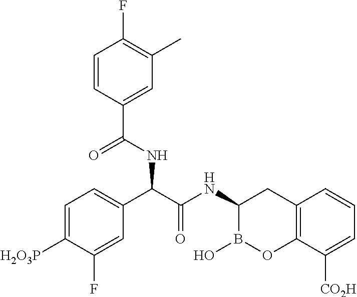 | (R)-3-((R)-2-(4-fluoro-3-methylbenzamido)-2-(3- fluoro-4-phosphonophenyl)acetamido)-2- hydroxy-3,4-dihydro-2H- benzo[e][1,2]oxaborinine-8-carboxylic acid | 574.24 |
| 88 | 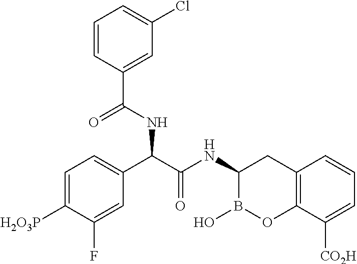 | (R)-3-((R)-2-(3-chlorobenzamido)-2-(3-fluoro-4- phosphonophenyl)acetamido)-2-hydroxy-3,4- dihydro-2H- benzo[e][1,2]oxaborinine-8-carboxylic acid | 576.66 |
| 89 | 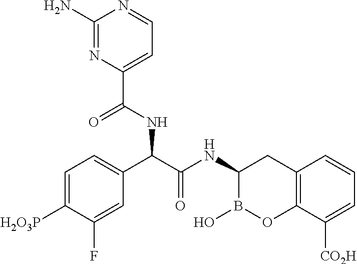 | (R)-3-((R)-2-(2-aminopyrimidine-4- carboxamido)-2-(3-fluoro-4- phosphonophenyl)acetamido)-2-hydroxy-3,4- dihydro-2H- benzo[e][1,2]oxaborinine-8-carboxylic acid | 559.21 |
| 90 | 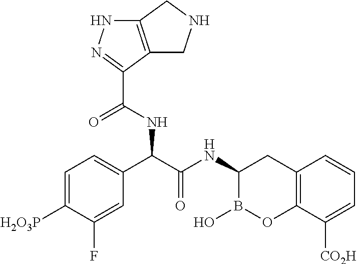 | (R)-3-((R)-2-(3-fluoro-4-phosphonophenyl)-2- (1,4,5,6-tetrahydropyrrolo[3,4-c]pyrazole-3- carboxamido)acetamido)-2-hydroxy-3,4-dihydro- 2H-benzo[e][1,2]oxaborinine-8-carboxylic acid | 573.24 |
| 91 | 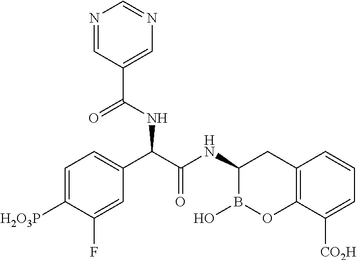 | (R)-3-((R)-2-(3-fluoro-4-phosphonophenyl)-2- (pyrimidine-5-carboxamido)acetamido)-2- hydroxy-3,4-dihydro-2H- benzo[e][1,2]oxaborinine-8-carboxylic acid | 544.20 |
| 92 | 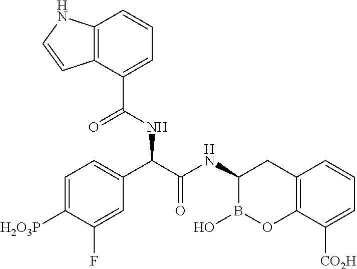 | (R)-3-((R)-2-(3-fluoro-4-phosphonophenyl)-2- (1H-indole-4-carboxamido)acetamido)-2- hydroxy-3,4-dihydro-2H- benzo[e][1,2]oxaborinine-8-carboxylic acid | 581.26 |
| 93 | 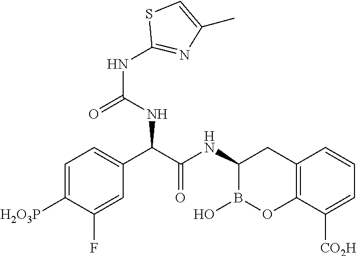 | (R)-3-((R)-2-(3-fluoro-4-phosphonophenyl)-2-(3- (4-methylthiazol-2-yl)ureido)acetamido)-2- hydroxy-3,4-dihydro-2H- benzo[e][1,2]oxaborinine-8-carboxylic acid | 578.27 |
| 94 | 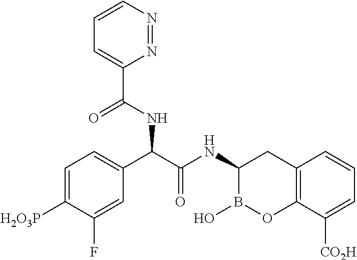 | (R)-3-((R)-2-(3-fluoro-4-phosphonophenyl)-2- (pyridazine-3-carboxamido)acetamido)-2- hydroxy-3,4-dihydro-2H- benzo[e][1,2]oxaborinine-8-carboxylic acid | 544.20 |
| 95 | 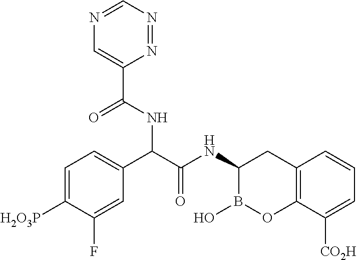 | (3R)-3-(2-(3-fluoro-4-phosphonophenyl)-2- (1,2,4-triazine-6-carboxamido)acetamido)-2- hydroxy-3,4-dihydro-2H- benzo[e][1,2]oxaborinine-8-carboxylic acid | 545.18 |
| 96 | 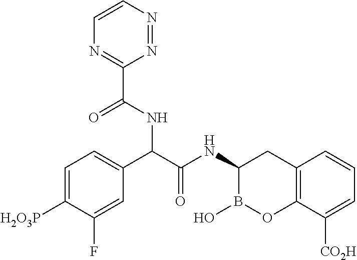 | (3R)-3-(2-(3-fluoro-4-phosphonophenyl)-2- (1,2,4-triazine-3-carboxamido)acetamido)-2- hydroxy-3,4-dihydro-2H- benzo[e][1,2]oxaborinine-8-carboxylic acid | 545.18 |
| 97 | 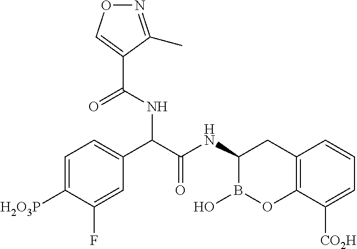 | (3R)-3-(2-(3-fluoro-4-phosphonophenyl)-2-(3- methylisoxazole-4-carboxamido)acetamido)-2- hydroxy-3,4-dihydro-2H- benzo[e][1,2]oxaborinine-8-carboxylic acid | 547.20 |
| 98 | 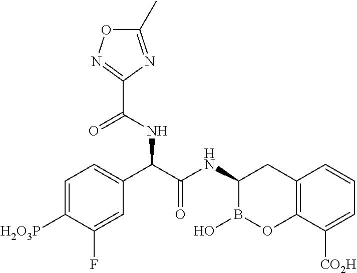 | (R)-3-((R)-2-(3-fluoro-4-phosphonophenyl)-2-(5- methyl-1,2,4-oxadiazole-3- carboxamido)acetamido)-2-hydroxy-3,4-dihydro- 2H-benzo[e][1,2]oxaborinine-8-carboxylic acid | 548.18 |
| 99 | 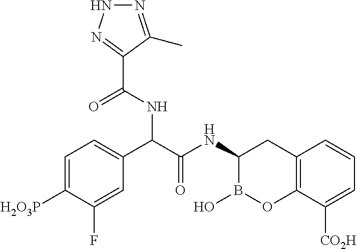 | (3R)-3-(2-(3-fluoro-4-phosphonophenyl)-2-(5- methyl-2H-1,2,3-triazole-4- carboxamido)acetamido)-2-hydroxy-3,4-dihydro- 2H-benzo[e][1,2]oxaborinine-8-carboxylic acid | 547.20 |
| 100 | 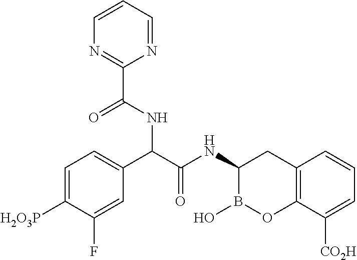 | (3R)-3-(2-(3-fluoro-4-phosphonophenyl)-2- (pyrimidine-2-carboxamido)acetamido)-2- hydroxy-3,4-dihydro-2H- benzo[e][1,2]oxaborinine-8-carboxylic acid | 544.20 |
| 101 | 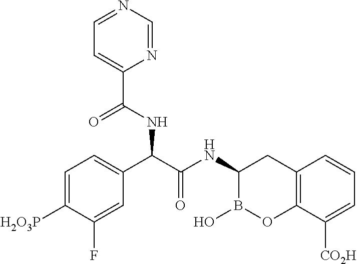 | (R)-3-((R)-2-(3-fluoro-4-phosphonophenyl)-2- (pyrimidine-4-carboxamido)acetamido)-2- hydroxy-3,4-dihydro-2H- benzo[e][1,2]oxaborinine-8-carboxylic acid | 544.20 |
| 102 | 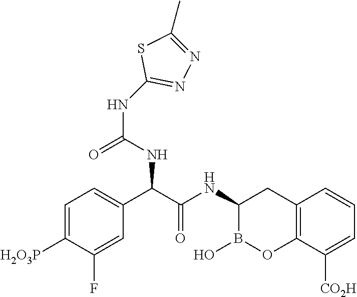 | (R)-3-((R)-2-(3-fluoro-4-phosphonophenyl)-2-(3- (5-methyl-1,3,4-thiadiazol-2- yl)ureido)acetamido)-2-hydroxy-3,4-dihydro-2H- benzo[e][1,2]oxaborinine-8-carboxylic acid | 579.26 |
| 103 | 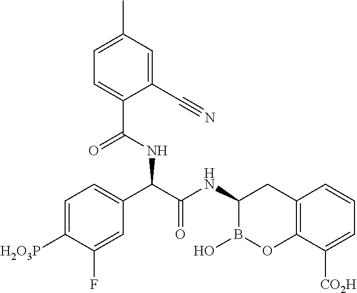 | (R)-3-((R)-2-(2-cyano-4-methylbenzamido)-2-(3- fluoro-4-phosphonophenyl)acetamido)-2- hydroxy-3,4-dihydro-2H- benzo[e][1,2]oxaborinine-8-carboxylic acid | 581.26 |
| 104 | 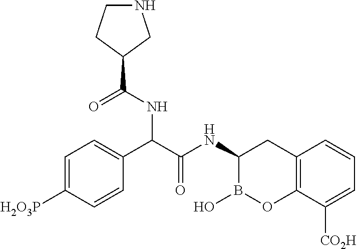 | (3R)-2-hydroxy-3-(2-(4-phosphonophenyl)-2- ((S)-pyrrolidine-3-carboxamido)acetamido)-3,4- dihydro-2H- benzo[e][1,2]oxaborinine-8-carboxylic acid | 517.24 |
| 105 | 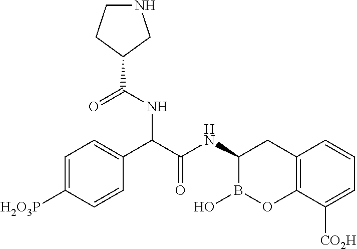 | (3R)-2-hydroxy-3-(2-(4-phosphonophenyl)-2- ((R)-pyrrolidine-3-carboxamido)acetamido)-3,4- dihydro-2H- benzo[e][1,2]oxaborinine-8-carboxylic acid | 517.24 |
| 106 | 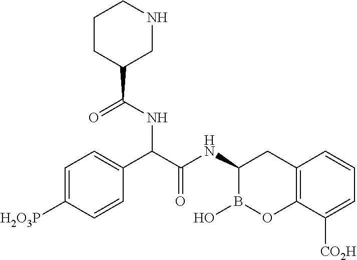 | (3R)-2-hydroxy-3-(2-(4-phosphonophenyl)-2- ((R)-piperidine-3-carboxamido)acetamido)-3,4- dihydro-2H- benzo[e][1,2]oxaborinine-8-carboxylic acid | 531.26 |
| 107 | 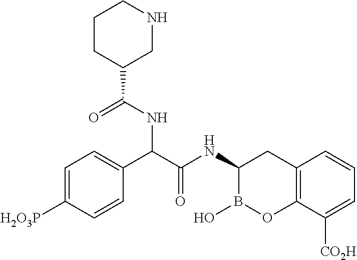 | (3R)-2-hydroxy-3-(2-(4-phosphonophenyl)-2- ((R)-piperidine-3-carboxamido)acetamido)-3,4- dihydro-2H- benzo[e][1,2]oxaborinine-8-carboxylic acid | 531.26 |
| 108 | 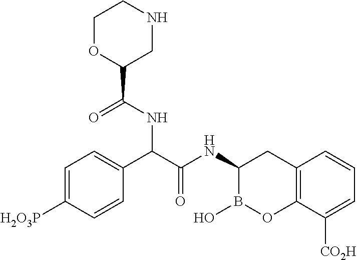 | (3R)-2-hydroxy-3-(2-((R)-morpholine-2- carboxamido)-2-(4- phosphonophenyl)acetamido)-3,4-dihydro-2H- benzo[e][1,2]oxaborinine-8-carboxylic acid | 533.24 |
| 109 | 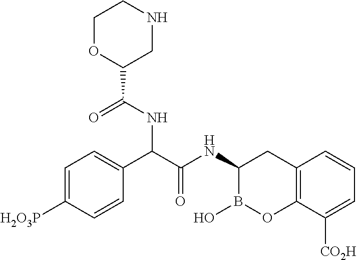 | (3R)-2-hydroxy-3-(2-((R)-morpholine-2- carboxamido)-2-(4- phosphonophenyl)acetamido)-3,4-dihydro-2H- benzo[e][1,2]oxaborinine-8-carboxylic acid | 533.24 |
| 110 | 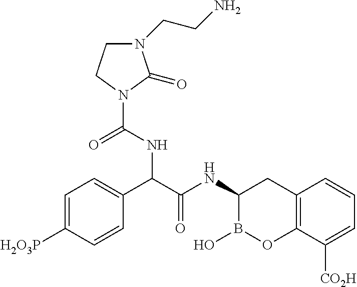 | (3R)-3-(2-(3-(2-aminoethyl)-2-oxoimidazolidine- 1-carboxamido)-2-(4- phosphonophenyl)acetamido)-2-hydroxy-3,4- dihydro-2H- benzo[e][1,2]oxaborinine-8-carboxylic acid | 575.28 |
| 111 | 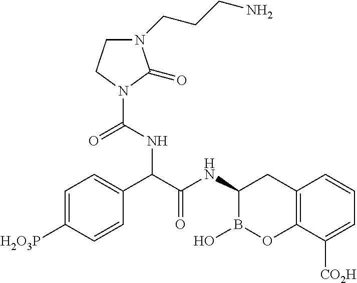 | (3R)-3-(2-(3-(3-aminopropyl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 589.30 |
| 112 | 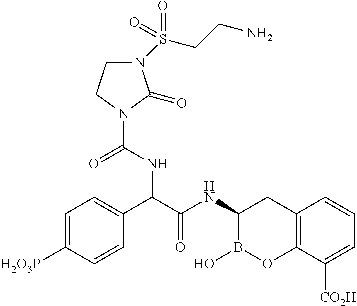 | (3R)-3-(2-(3-((2-aminoethyl)sulfonyl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 639.34 |
| 113 | 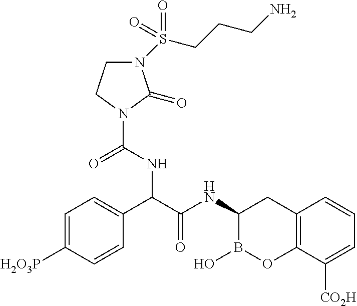 | (3R)-3-(2-(3-((3-aminopropyl)sulfonyl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 653.36 |
| 115 | 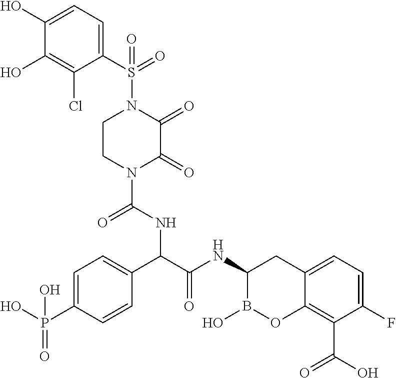 [SMILES: O=C(O)c1c(F)ccc2c1OB(O)[C@@H](NC(=O)C(NC(=O)N1CCN(S(=O)(=O)c3ccc(O)c(O)c3Cl)C(=O)C1=O)c1ccc(P(=O)(O)O)cc1)C2] | (3R)-3-(2-(4-((2-chloro-3,4- dihydroxyphenyl)sulfonyl)-2,3-dioxopiperazine- 1-carboxamido)-2-(4- phosphonophenyl)acetamido)-7-fluoro-2- hydroxy-3,4-dihydro-2H- benzo[e][1,2]oxaborinine-8-carboxylic acid | 784.81 |
| 116 | 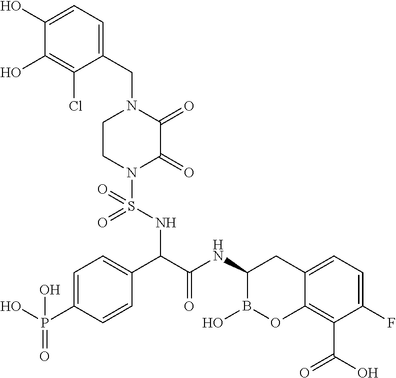 [SMILES: O=C(O)c1c(F)ccc2c1OB(O)[C@@H](NC(=O)C(NS(=O)(=O)N1CCN(Cc3ccc(O)c(O)c3Cl)C(=O)C1=O)c1ccc(P(=O)(O)O)cc1)C2] | (3R)-3-(2-((4-(2-chloro-3,4-dihydroxybenzyl)- 2,3-dioxopiperazine)-1-sulfonamido)-2-(4- phosphonophenyl)acetamido)-7-fluoro-2- hydroxy-3,4-dihydro-2H- benzo[e][1,2]oxaborinine-8-carboxylic acid | 770.82 |
| 117 | 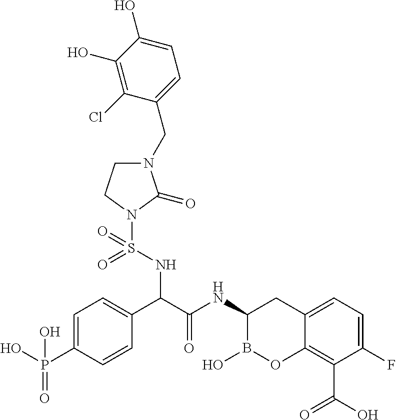 [SMILES: O=C(O)c1c(F)ccc2c1OB(O)[C@@H](NC(=O)C(NS(=O)(=O)N1CCN(Cc3ccc(O)c(O)c3Cl)C1=O)c1ccc(P(=O)(O)O)cc1)C2] | (3R)-3-(2-((3-(2-chloro-3,4-dihydroxybenzyl)-2- oxoimidazolidine)-1-sulfonamido)-2-(4- phosphonophenyl)acetamido)-7-fluoro-2- hydroxy-3,4-dihydro-2H- benzo[e][1,2]oxaborinine-8-carboxylic acid | 742.81 |
| 118 | 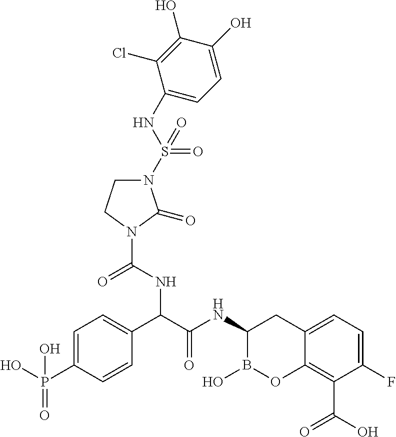 [SMILES: O=C(O)c1c(F)ccc2c1OB(O)[C@@H](NC(=O)C(NC(=O)N1CCN(S(=O)(=O)Nc3ccc(O)c(O)c3Cl)C1=O)c1ccc(P(=O)(O)O)cc1)C2] | (3R)-3-(2-(3-(N-(2-chloro-3,4- dihydroxyphenyl)sulfamoyl)-2-oxoimidazolidine- 1-carboxamido)-2-(4- phosphonophenyl)acetamido)-7-fluoro-2- hydroxy-3,4-dihydro-2H- benzo[e][1,2]oxaborinine-8-carboxylic acid | 771.81 |
| 119 | 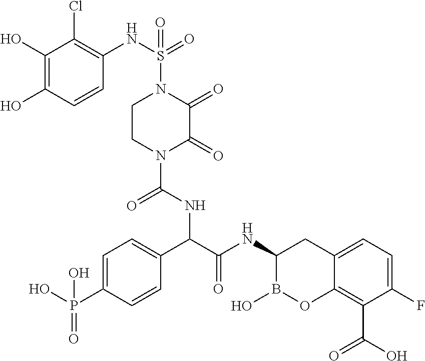 [SMILES: O=C(O)c1c(F)ccc2c1OB(O)[C@@H](NC(=O)C(NC(=O)N1CCN(S(=O)(=O)Nc3ccc(O)c(O)c3Cl)C(=O)C1=O)c1ccc(P(=O)(O)O)cc1)C2] | (3R)-3-(2-(4-(N-(2-chloro-3,4- dihydroxyphenyl)sulfamoyl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 799.82 |
| 121 | 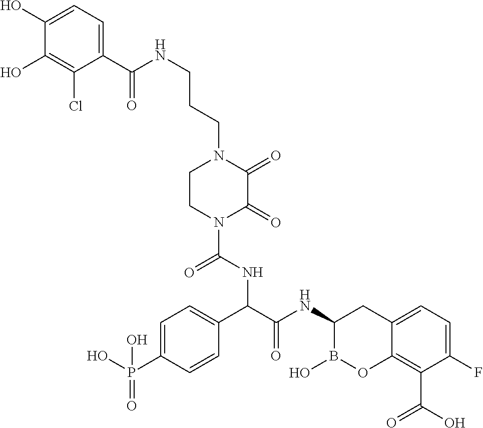 [SMILES: O=C(NCCCN1CCN(C(=O)NC(C(=O)N[C@H]2Cc3ccc(F)c(C(=O)O)c3OB2O)c2ccc(P(=O)(O)O)cc2)C(=O)C1=O)c1ccc(O)c(O)c1Cl] | (3R)-3-(2-(4-(3-(2-chloro-3,4- dihydroxybenzamido)propyl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 805.85 |
| 122 | 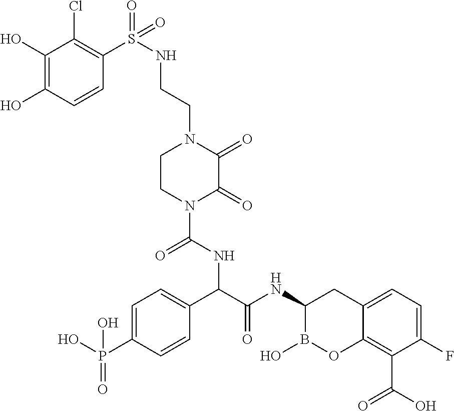 [SMILES: O=C(O)c1c(F)ccc2c1OB(O)[C@@H](NC(=O)C(NC(=O)N1CCN(CCNS(=O)(=O)c3ccc(O)c(O)c3Cl)C(=O)C1=O)c1ccc(P(=O)(O)O)cc1)C2] | (3R)-3-(2-(4-(2-((2-chloro-3,4- dihydroxyphenyl)sulfonamido)ethyl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 827.87 |
| 123 | 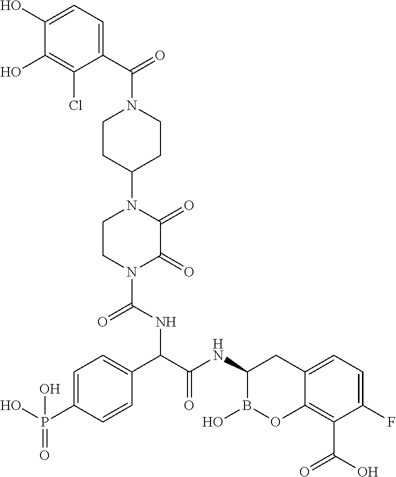 [SMILES: O=C(O)c1c(F)ccc2c1OB(O)[C@@H](NC(=O)C(NC(=O)N1CCN(C3CCN(C(=O)c4ccc(O)c(O)c4Cl)CC3)C(=O)C1=O)c1ccc(P(=O)(O)O)cc1)C2] | (3R)-3-(2-(4-(1-(2-chloro-3,4- dihydroxybenzoyl)piperidin-4-yl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 831.89 |
| 124 | 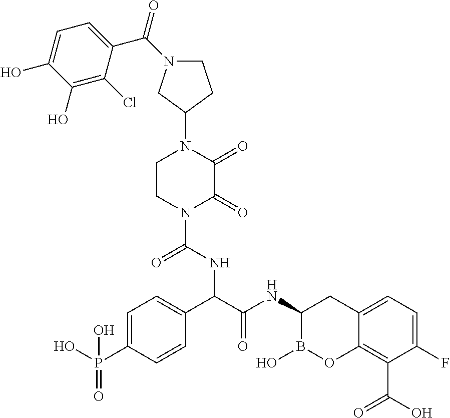 [SMILES: O=C(O)c1c(F)ccc2c1OB(O)[C@@H](NC(=O)C(NC(=O)N1CCN(C3CCN(C(=O)c4ccc(O)c(O)c4Cl)C3)C(=O)C1=O)c1ccc(P(=O)(O)O)cc1)C2] | (3R)-3-(2-(4-(1-(2-chloro-3,4- dihydroxybenzoyl)pyrrolidin-3-yl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 817.86 |
| 125 | 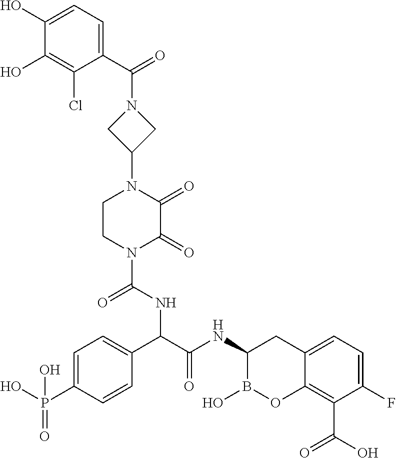 [SMILES: O=C(O)c1c(F)ccc2c1OB(O)[C@@H](NC(=O)C(NC(=O)N1CCN(C3CN(C(=O)c4ccc(O)c(O)c4Cl)C3)C(=O)C1=O)c1ccc(P(=O)(O)O)cc1)C2] | (3R)-3-(2-(4-(1-(2-chloro-3,4- dihydroxybenzoyl)azetidin-3-yl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 803.84 |
| 126 | 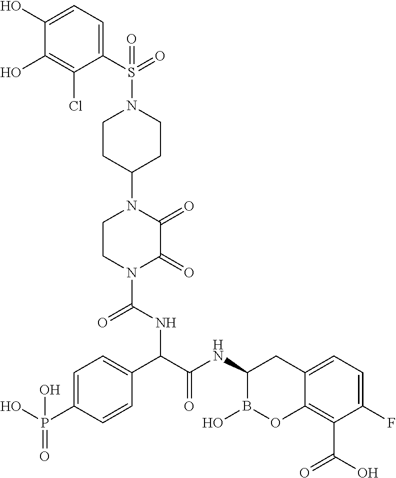 [SMILES: O=C(O)c1c(F)ccc2c1OB(O)[C@@H](NC(=O)C(NC(=O)N1CCN(C3CCN(S(=O)(=O)c4ccc(O)c(O)c4Cl)CC3)C(=O)C1=O)c1ccc(P(=O)(O)O)cc1)C2] | (3R)-3-(2-(4-(1-((2-chloro-3,4- dihydroxyphenyl)sulfonyl)piperidin-4-yl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 867.94 |
| 127 | 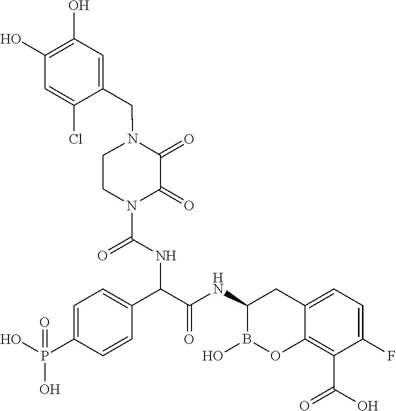 [SMILES: O=C(O)c1c(F)ccc2c1OB(O)[C@@H](NC(=O)C(NC(=O)N1CCN(Cc3cc(O)c(O)cc3Cl)C(=O)C1=O)c1ccc(P(=O)(O)O)cc1)C2] | (3R)-3-(2-(4-(2-chloro-4,5-dihydroxybenzyl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 734.77 |
| 128 | 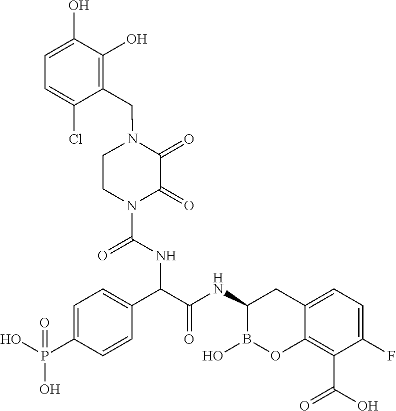 [SMILES: O=C(O)c1c(F)ccc2c1OB(O)[C@@H](NC(=O)C(NC(=O)N1CCN(Cc3c(Cl)ccc(O)c3O)C(=O)C1=O)c1ccc(P(=O)(O)O)cc1)C2] | (3R)-3-(2-(4-(6-chloro-2,3-dihydroxybenzyl)-2,3- dioxopiperazine-1-carboxamido)-2-(4- phosphonophenyl)acetamido)-7-fluoro-2- hydroxy-3,4-dihydro-2H- benzo[e][1,2]oxaborinine-8-carboxylic acid | 734.77 |
| 129 | 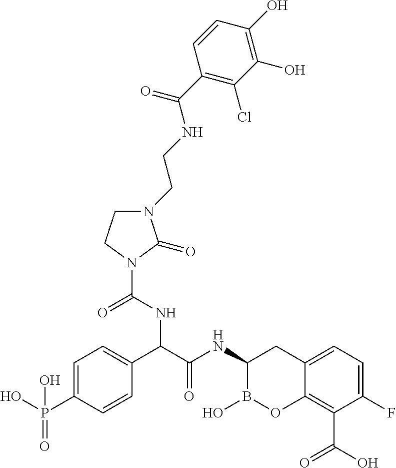 [SMILES: O=C(NCCN1CCN(C(=O)NC(C(=O)N[C@H]2Cc3ccc(F)c(C(=O)O)c3OB2O)c2ccc(P(=O)(O)O)cc2)C1=O)c1ccc(O)c(O)c1Cl] | (3R)-3-(2-(3-(2-(2-chloro-3,4- dihydroxybenzamido)ethyl)-2-oxoimidazolidine- 1-carboxamido)-2-(4- phosphonophenyl)acetamido)-7-fluoro-2- hydroxy-3,4-dihydro-2H- benzo[e][1,2]oxaborinine-8-carboxylic acid | 763.82 |
| 130 | 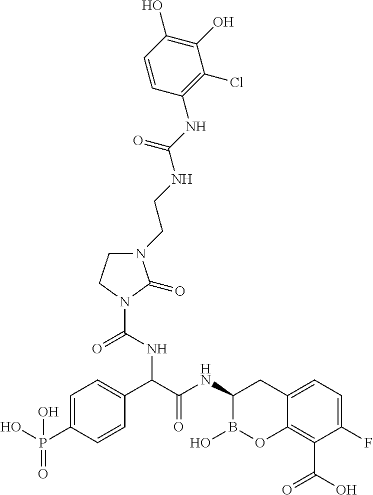 [SMILES: O=C(NCCN1CCN(C(=O)NC(C(=O)N[C@H]2Cc3ccc(F)c(C(=O)O)c3OB2O)c2ccc(P(=O)(O)O)cc2)C1=O)Nc1ccc(O)c(O)c1Cl] | (3R)-3-(2-(3-(2-(3-(2-chloro-3,4- dihydroxyphenyl)ureido)ethyl)-2- oxoimidazolidine-1-carboxamido)-2-(4- phosphonophenyl)acetamido)-7-fluoro-2- hydroxy-3,4-dihydro-2H- benzo[e][1,2]oxaborinine-8-carboxylic acid | 778.83 |
| 131 | 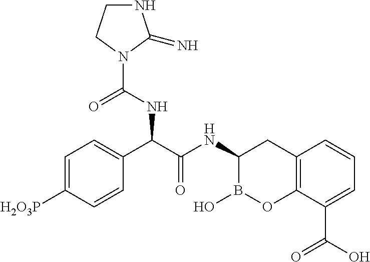 | (R)-2-hydroxy-3-((R)-2-(2-iminoimidazolidine-1- carboxamido)-2-(4- phosphonophenyl)acetamido)-3,4-dihydro-2H- benzo[e][1,2]oxaborinine-8-carboxylic acid | 531.22 |
| 132 | 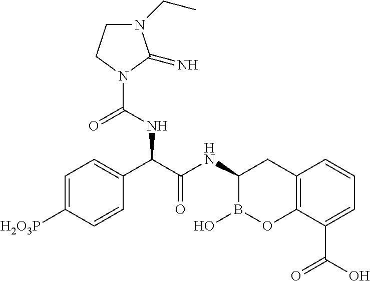 | (R)-3-((R)-2-(3-ethyl-2-iminoimidazolidine-1- carboxamido)-2-(4- phosphonophenyl)acetamido)-2-hydroxy-3,4- dihydro-2H- benzo[e][1,2]oxaborinine-8-carboxylic acid | 559.28 |
| 133 | 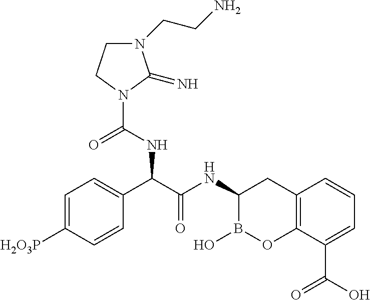 | (R)-3-((R)-2-(3-(2-aminoethyl)-2- iminoimidazolidine-1-carboxamido)-2-(4- phosphonophenyl)acetamido)-2-hydroxy-3,4- dihydro-2H- benzo[e][1,2]oxaborinine-8-carboxylic acid | 574.29 |
| 134 | 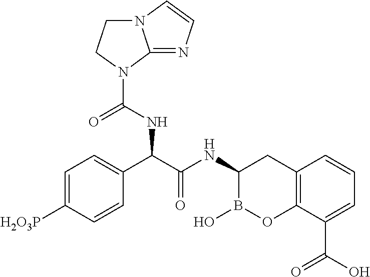 | (R)-3-((R)-2-(2,3-dihydro-1H-imidazo[1,2- a]imidazole-1-carboxamido)-2-(4- phosphonophenyl)acetamido)-2-hydroxy-3,4- dihydro-2H- benzo[e][1,2]oxaborinine-8-carboxylic acid | 555.25 |
| 135 | 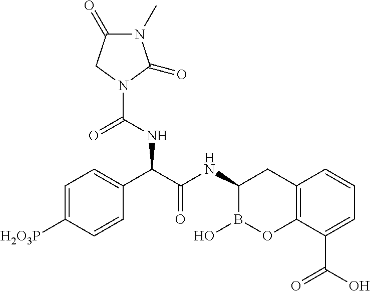 | (R)-2-hydroxy-3-((R)-2-(3-methyl-2,4- dioxoimidazolidine-1-carboxamido)-2-(4- phosphonophenyl)acetamido)-3,4-dihydro-2H- benzo[e][1,2]oxaborinine-8-carboxylic acid | 560.22 |
| 136 | 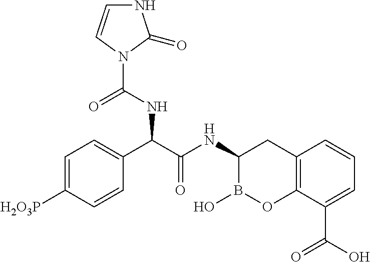 | (R)-2-hydroxy-3-((R)-2-(2-oxo-2,3-dihydro-1H- imidazole-1-carboxamido)-2-(4- phosphonophenyl)acetamido)-3,4-dihydro-2H- benzo[e][1,2]oxaborinine-8-carboxylic acid | 530.19 |
| 137 | 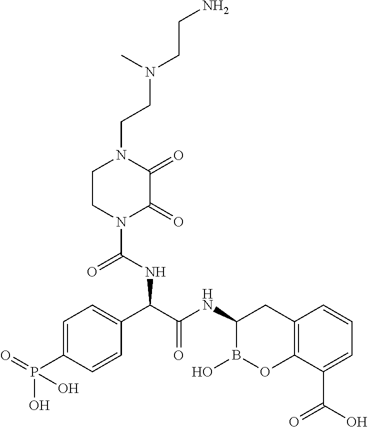 [SMILES: CN(CCN)CCN1CCN(C(=O)N[C@@H](C(=O)N[C@H]2Cc3cccc(C(=O)O)c3OB2O)c2ccc(P(=O)(O)O)cc2)C(=O)C1=O] | (R)-3-((R)-2-(4-(2-((2- aminoethyl)(methyl)amino)ethyl)-2,3- dioxopiperazine-1-carboxamido)-2-(4- phosphonophenyl)acetamido)-2-hydroxy-3,4- dihydro-2H- benzo[e][1,2]oxaborinine-8-carboxylic acid | 660.38 |
| 138 | 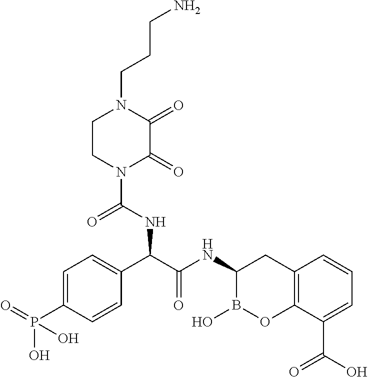 [SMILES: NCCCN1CCN(C(=O)N[C@@H](C(=O)N[C@H]2Cc3cccc(C(=O)O)c3OB2O)c2ccc(P(=O)(O)O)cc2)C(=O)C1=O] | (R)-3-((R)-2-(4-(3-aminopropyl)-2,3- dioxopiperazine-1-carboxamido)-2-(4- phosphonophenyl)acetamido)-2-hydroxy-3,4- dihydro-2H- benzo[e][1,2]oxaborinine-8-carboxylic acid | 617.31 |
| 139 | 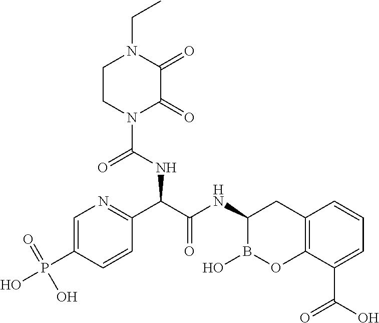 [SMILES: CCN1CCN(C(=O)N[C@@H](C(=O)N[C@H]2Cc3cccc(C(=O)O)c3OB2O)c2ccc(P(=O)(O)O)cn2)C(=O)C1=O] | (R)-3-((R)-2-(4-ethyl-2,3-dioxopiperazine-1- carboxamido)-2-(5-phosphonopyridin-2- yl)acetamido)-2-hydroxy-3,4-dihydro-2H- benzo[e][1,2]oxaborinine-8-carboxylic acid | 589.26 |
| 140 | 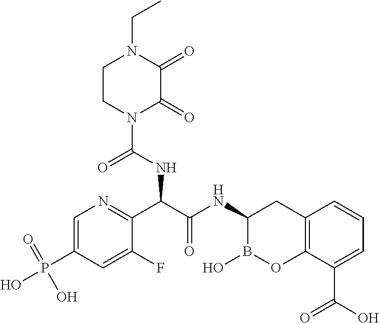 [SMILES: CCN1CCN(C(=O)N[C@@H](C(=O)N[C@H]2Cc3cccc(C(=O)O)c3OB2O)c2ncc(P(=O)(O)O)cc2F)C(=O)C1=O] | (R)-3-((R)-2-(4-ethyl-2,3-dioxopiperazine-1- carboxamido)-2-(3-fluoro-5-phosphonopyridin-2- yl)acetamido)-2-hydroxy-3,4-dihydro-2H- benzo[e][1,2]oxaborinine-8-carboxylic acid | 607.25 |
| 141 | 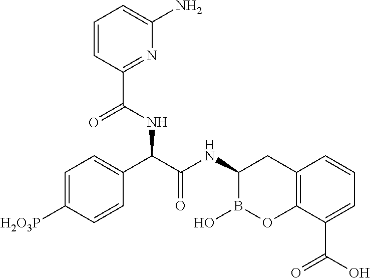 | (R)-3-((R)-2-(6-aminopicolinamido)-2-(4- phosphonophenyl)acetamido)-2-hydroxy-3,4- dihydro-2H- benzo[e][1,2]oxaborinine-8-carboxylic acid | 540.23 |
| 142 | 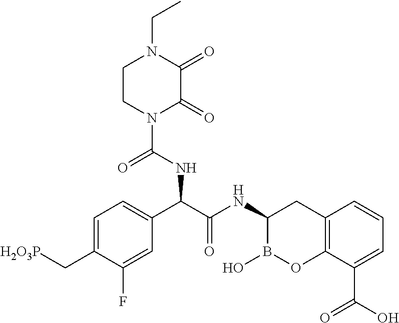 | (R)-3-((R)-2-(4-ethyl-2,3-dioxopiperazine-1- carboxamido)-2-(3-fluoro-4- (phosphonomethyl)phenyl)acetamido)-2- hydroxy-3,4-dihydro-2H- benzo[e][1,2]oxaborinine-8-carboxylic acid | 620.29 |
| 143 | 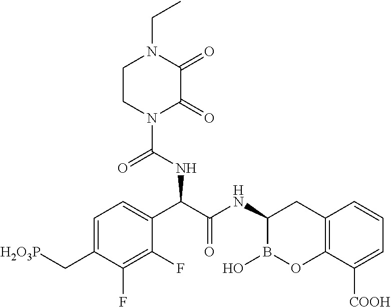 | (R)-3-((R)-2-(2,3-difluoro-4- (phosphonomethyl)phenyl)-2-(4-ethyl-2,3- dioxopiperazine-1-carboxamido)acetamido)-2- hydroxy-3,4-dihydro-2H- benzo[e][1,2]oxaborinine-8-carboxylic acid | 638.28 |
| 144 | 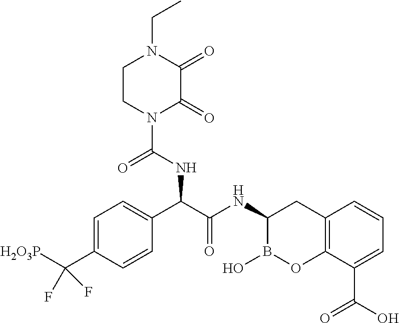 | (R)-3-((R)-2-(4- (difluoro(phosphono)methyl)phenyl)-2-(4-ethyl- 2,3-dioxopiperazine-1-carboxamido)acetamido)- 2-hydroxy-3,4-dihydro-2H- benzo[e][1,2]oxaborinine-8-carboxylic acid | 638.28 |
| 145 | 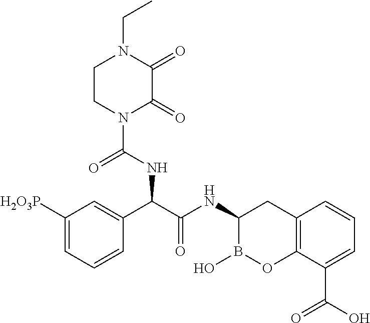 | (R)-3-((R)-2-(4-ethyl-2,3-dioxopiperazine-1- carboxamido)-2-(3- phosphonophenyl)acetamido)-2-hydroxy-3,4- dihydro-2H- benzo[e][1,2]oxaborinine-8-carboxylic acid | 588.27 |
| 146 | 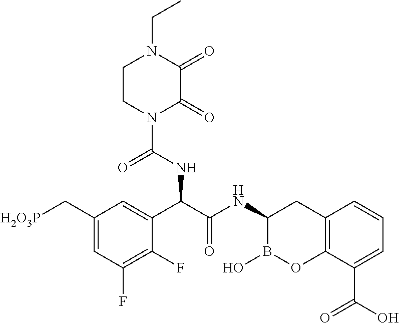 | (R)-3-((R)-2-(2,3-difluoro-5- (phosphonomethyl)phenyl)-2-(4-ethyl-2,3- dioxopiperazine-1-carboxamido)acetamido)-2- hydroxy-3,4-dihydro-2H- benzo[e][1,2]oxaborinine-8-carboxylic acid | 638.28 |
| 147 | 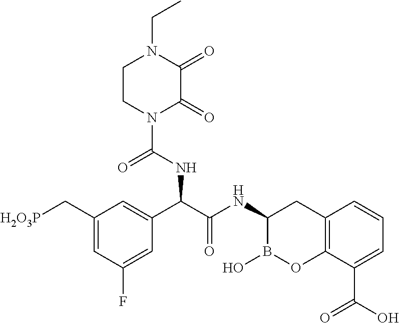 | (R)-3-((R)-2-(4-ethyl-2,3-dioxopiperazine-1- carboxamido)-2-(3-fluoro-5- (phosphonomethyl)phenyl)acetamido)-2- hydroxy-3,4-dihydro-2H- benzo[e][1,2]oxaborinine-8-carboxylic acid | 620.29 |
| 148 | 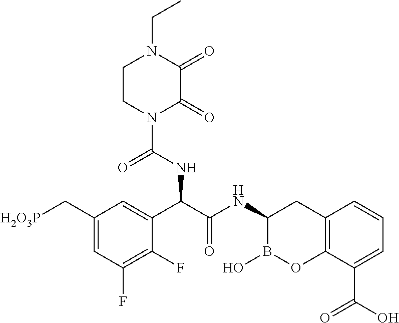 | (R)-3-((R)-2-(2,3-difluoro-5- (phosphonomethyl)phenyl)-2-(4-ethyl-2,3- dioxopiperazine-1-carboxamido)acetamido)-2- hydroxy-3,4-dihydro-2H- benzo[e][1,2]oxaborinine-8-carboxylic acid | 638.28 |
| 149 | 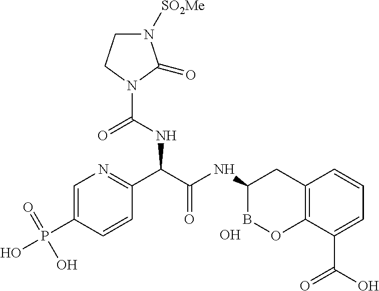 | (R)-2-hydroxy-3-((R)-2-(3-(methylsulfonyl)-2- oxoimidazolidine-1-carboxamido)-2-(5- phosphonopyridin-2-yl)acetamido)-3,4-dihydro- 2H-benzo[e][1,2]oxaborinine-8-carboxylic acid | 611.28 |
| 150 | 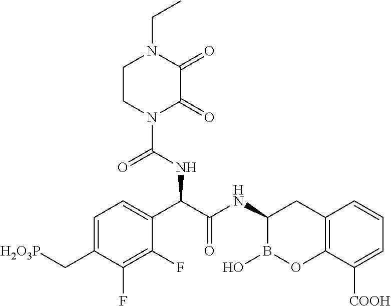 | (R)-3-((R)-2-(2,3-difluoro-4- (phosphonomethyl)phenyl)-2-(3- (methylsulfonyl)-2-oxoimidazolidine-1- carboxamido)acetamido)-2-hydroxy-3,4-dihydro- 2H-benzo[e][1,2]oxaborinine-8-carboxylic acid | 660.30 |
| 151 | 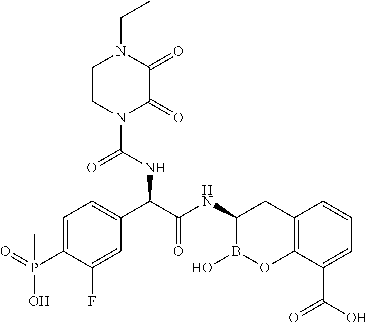 [SMILES: CCN1CCN(C(=O)N[C@@H](C(=O)N[C@H]2Cc3cccc(C(=O)O)c3OB2O)c2ccc(P(C)(=O)O)c(F)c2)C(=O)C1=O] | (3R)-3-((2R)-2-(4-ethyl-2,3-dioxopiperazine-1- carboxamido)-2-(3-fluoro-4- (hydroxy(methyl)phosphoryl)phenyl)acetamido)- 2-hydroxy-3,4-dihydro-2H- benzo[e][1,2]oxaborinine-8-carboxylic acid | 604.29 |
| 152 | 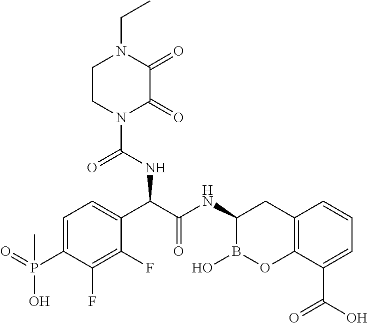 [SMILES: CCN1CCN(C(=O)N[C@@H](C(=O)N[C@H]2Cc3cccc(C(=O)O)c3OB2O)c2ccc(P(C)(=O)O)c(F)c2F)C(=O)C1=O] | (3R)-3-((2R)-2-(2,3-difluoro-4- (hydroxy(methyl)phosphoryl)phenyl)-2-(4-ethyl- 2,3-dioxopiperazine-1-carboxamido)acetamido)- 2-hydroxy-3,4-dihydro-2H- benzo[e][1,2]oxaborinine-8-carboxylic acid | 622.28 |
| 153 | 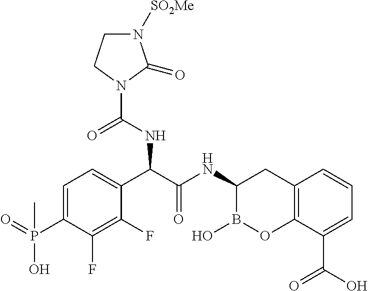 [SMILES: CP(=O)(O)c1ccc([C@@H](NC(=O)N2CCN(S(C)(=O)=O)C2=O)C(=O)N[C@H]2Cc3cccc(C(=O)O)c3OB2O)c(F)c1F] | (3R)-3-((2R)-2-(2,3-difluoro-4- (hydroxy(methyl)phosphoryl)phenyl)-2-(3- (methylsulfonyl)-2-oxoimidazolidine-1- carboxamido)acetamido)-2-hydroxy-3,4-dihydro- 2H-benzo[e][1,2]oxaborinine-8-carboxylic acid | 644.30 |
| 154 | 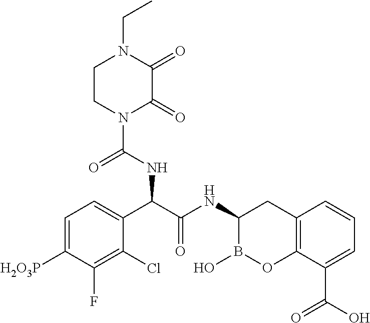 | (R)-3-((R)-2-(2-chloro-3-fluoro-4- phosphonophenyl)-2-(4-ethyl-2,3- dioxopiperazine-1-carboxamido)acetamido)-2- hydroxy-3,4-dihydro-2H- benzo[e][1,2]oxaborinine-8-carboxylic acid | 640.71 |
| 155 | 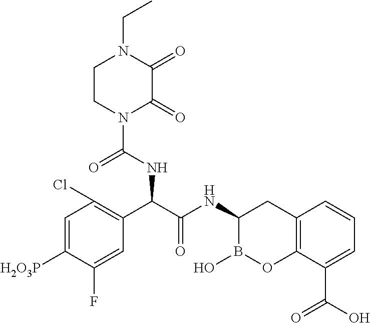 | (R)-3-((R)-2-(2-chloro-5-fluoro-4- phosphonophenyl)-2-(4-ethyl-2,3- dioxopiperazine-1-carboxamido)acetamido)-2- hydroxy-3,4-dihydro-2H- benzo[e][1,2]oxaborinine-8-carboxylic acid | 640.71 |
| 156 | 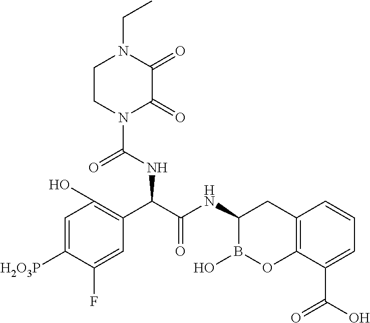 | (R)-3-((R)-2-(4-ethyl-2,3-dioxopiperazine-1- carboxamido)-2-(5-fluoro-2-hydroxy-4- phosphonophenyl)acetamido)-2-hydroxy-3,4- dihydro-2H- benzo[e][1,2]oxaborinine-8-carboxylic acid | 622.26 |
| 157 | 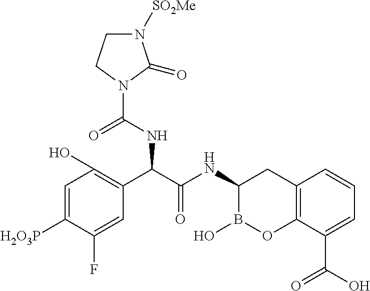 | (R)-3-((R)-2-(5-fluoro-2-hydroxy-4- phosphonophenyl)-2-(3-(methylsulfonyl)-2- oxoimidazolidine-1-carboxamido)acetamido)-2- hydroxy-3,4-dihydro-2H- benzo[e][1,2]oxaborinine-8-carboxylic acid | 644.28 |
| 158 | 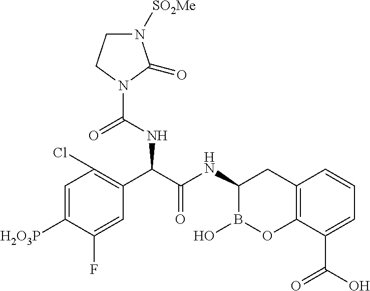 | (R)-3-((R)-2-(2-chloro-5-fluoro-4- phosphonophenyl)-2-(3-(methylsulfonyl)-2- oxoimidazolidine-1-carboxamido)acetamido)-2- hydroxy-3,4-dihydro-2H- benzo[e][1,2]oxaborinine-8-carboxylic acid | 662.73 |
| 159 | 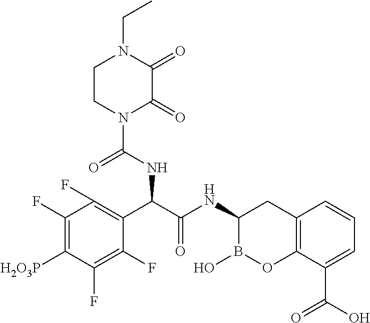 | (R)-3-((R)-2-(4-ethyl-2,3-dioxopiperazine-1- carboxamido)-2-(2,3,5,6-tetrafluoro-4- phosphonophenyl)acetamido)-2-hydroxy-3,4- dihydro-2H- benzo[e][1,2]oxaborinine-8-carboxylic acid | 660.23 |
| 507 | 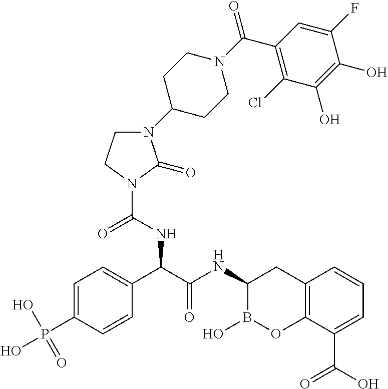 [SMILES: O=C(O)c1cccc2c1OB(O)[C@@H](NC(=O)[C@H](NC(=O)N1CCN(C3CCN(C(=O)c4cc(F)c(O)c(O)c4Cl)CC3)C1=O)c1ccc(P(=O)(O)O)cc1)C2] | (R)-3-((R)-2-(3-(1-(2-chloro-5-fluoro-3,4- dihydroxybenzoyl)piperidin-4-yl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 803.88 |
| 508 | 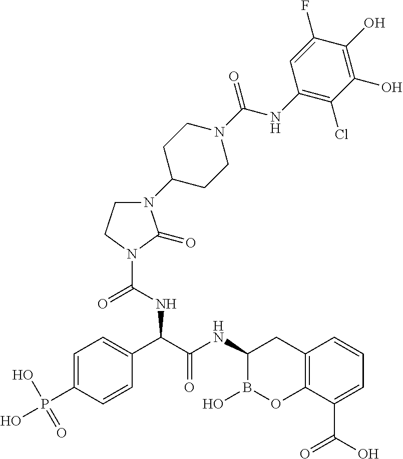 [SMILES: O=C(O)c1cccc2c1OB(O)[C@@H](NC(=O)[C@H](NC(=O)N1CCN(C3CCN(C(=O)Nc4cc(F)c(O)c(O)c4Cl)CC3)C1=O)c1ccc(P(=O)(O)O)cc1)C2] | (R)-3-((R)-2-(3-(1-((2-chloro-5-fluoro-3,4- dihydroxyphenyl)carbamoyl)piperidin-4-yl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 818.90 |
| 510 | 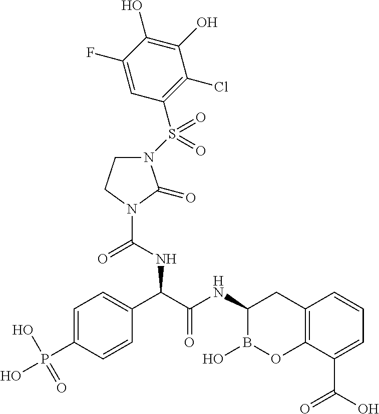 [SMILES: O=C(O)c1cccc2c1OB(O)[C@@H](NC(=O)[C@H](NC(=O)N1CCN(S(=O)(=O)c3cc(F)c(O)c(O)c3Cl)C1=O)c1ccc(P(=O)(O)O)cc1)C2] | (R)-3-((R)-2-(3-((2-chloro-5-fluoro-3,4- dihydroxyphenyl)sulfonyl)-2-oxoimidazolidine- 1-carboxamido)-2-(4- phosphonophenyl)acetamido)-2-hydroxy-3,4- dihydro-2H- benzo[e][1,2]oxaborinine-8-carboxylic acid | 756.80 |
| 511 | 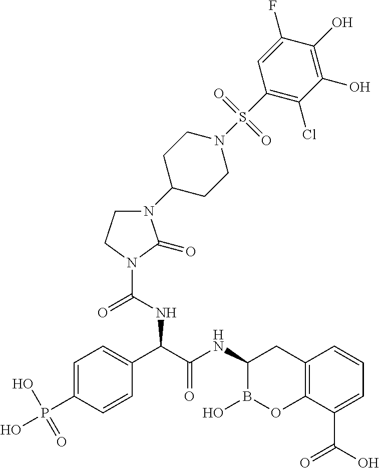 [SMILES: O=C(O)c1cccc2c1OB(O)[C@@H](NC(=O)[C@H](NC(=O)N1CCN(C3CCN(S(=O)(=O)c4cc(F)c(O)c(O)c4Cl)CC3)C1=O)c1ccc(P(=O)(O)O)cc1)C2] | (R)-3-((R)-2-(3-(1-((2-chloro-5-fluoro-3,4- dihydroxyphenyl)sulfonyl)piperidin-4-yl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 839.93 |
| 512 | 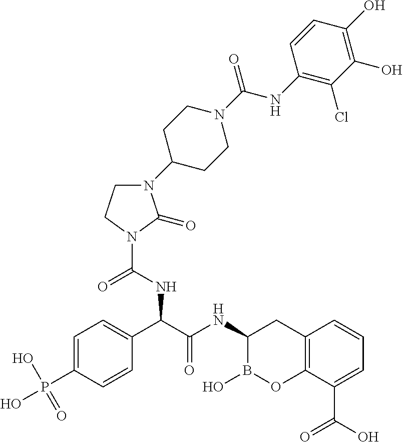 [SMILES: O=C(O)c1cccc2c1OB(O)[C@@H](NC(=O)[C@H](NC(=O)N1CCN(C3CCN(C(=O)Nc4ccc(O)c(O)c4Cl)CC3)C1=O)c1ccc(P(=O)(O)O)cc1)C2] | (R)-3-((R)-2-(3-(1-((2-chloro-3,4- dihydroxyphenyl)carbamoyl)piperidin-4-yl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 800.91 |
| 513 | 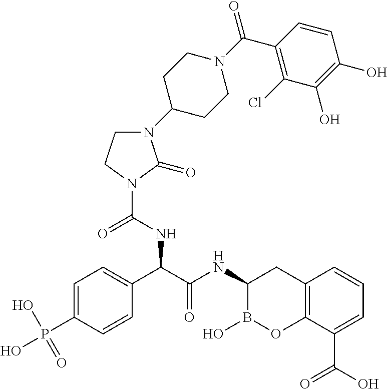 [SMILES: O=C(O)c1cccc2c1OB(O)[C@@H](NC(=O)[C@H](NC(=O)N1CCN(C3CCN(C(=O)c4ccc(O)c(O)c4Cl)CC3)C1=O)c1ccc(P(=O)(O)O)cc1)C2] | (R)-3-((R)-2-(3-(1-(2-chloro-3,4- dihydroxybenzoyl)piperidin-4-yl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 785.89 |
| 514 | 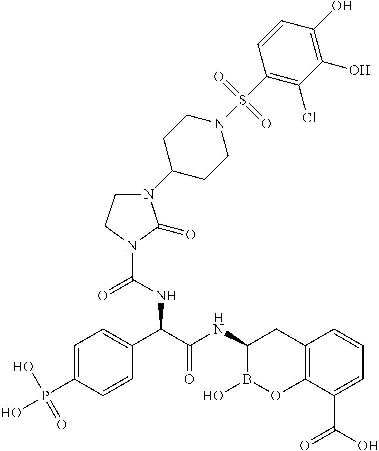 [SMILES: O=C(O)c1cccc2c1OB(O)[C@@H](NC(=O)[C@H](NC(=O)N1CCN(C3CCN(S(=O)(=O)c4ccc(O)c(O)c4Cl)CC3)C1=O)c1ccc(P(=O)(O)O)cc1)C2] | (R)-3-((R)-2-(3-(1-((2-chloro-3,4- dihydroxyphenyl)sulfonyl)piperidin-4-yl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 821.94 |
| 515 | 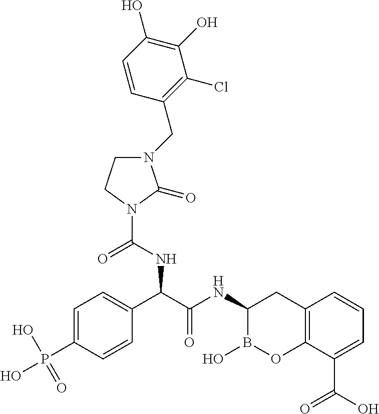 [SMILES: O=C(O)c1cccc2c1OB(O)[C@@H](NC(=O)[C@H](NC(=O)N1CCN(Cc3ccc(O)c(O)c3Cl)C1=O)c1ccc(P(=O)(O)O)cc1)C2] | (R)-3-((R)-2-(3-(2-chloro-3,4-dihydroxybenzyl)- 2-oxoimidazolidine-1-carboxamido)-2-(4- phosphonophenyl)acetamido)-2-hydroxy-3,4- dihydro-2H- benzo[e][1,2]oxaborinine-8-carboxylic acid | 688.77 |
| 516 | 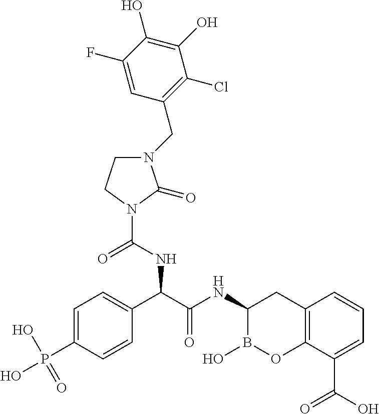 [SMILES: O=C(O)c1cccc2c1OB(O)[C@@H](NC(=O)[C@H](NC(=O)N1CCN(Cc3cc(F)c(O)c(O)c3Cl)C1=O)c1ccc(P(=O)(O)O)cc1)C2] | (R)-3-((R)-2-(3-(2-chloro-5-fluoro-3,4- dihydroxybenzyl)-2-oxoimidazolidine-1- carboxamido)-2-(4- phosphonophenyl)acetamido)-2-hydroxy-3,4- dihydro-2H- benzo[e][1,2]oxaborinine-8-carboxylic acid | 706.76 |
| 518 | 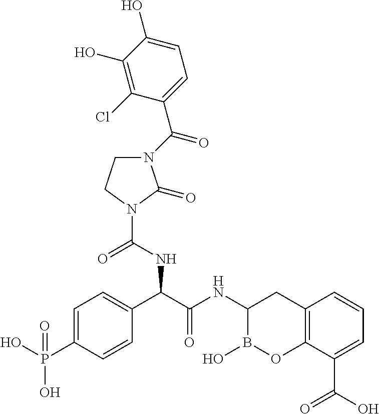 [SMILES: O=C(O)c1cccc2c1OB(O)C(NC(=O)[C@H](NC(=O)N1CCN(C(=O)c3ccc(O)c(O)c3Cl)C1=O)c1ccc(P(=O)(O)O)cc1)C2] | 3-((R)-2-(3-(2-chloro-3,4-dihydroxybenzoyl)-2- oxoimidazolidine-1-carboxamido)-2-(4- phosphonophenyl)acetamido)-2-hydroxy-3,4- dihydro-2H- benzo[e][1,2]oxaborinine-8-carboxylic acid | 702.76 |
| 519 | 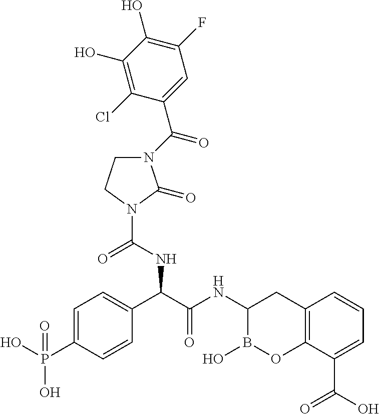 [SMILES: O=C(O)c1cccc2c1OB(O)C(NC(=O)[C@H](NC(=O)N1CCN(C(=O)c3cc(F)c(O)c(O)c3Cl)C1=O)c1ccc(P(=O)(O)O)cc1)C2] | 3-((R)-2-(3-(2-chloro-5-fluoro-3,4- dihydroxybenzoyl)-2-oxoimidazolidine-1- carboxamido)-2-(4- phosphonophenyl)acetamido)-2-hydroxy-3,4- dihydro-2H- benzo[e][1,2]oxaborinine-8-carboxylic acid | 720.75 |
| 520 | 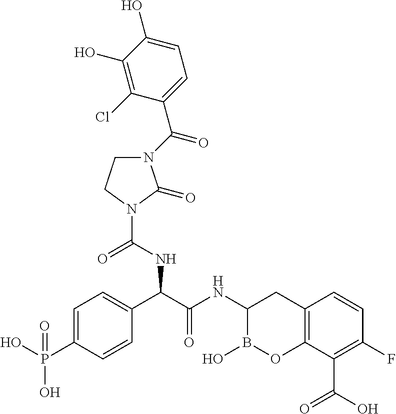 [SMILES: O=C(O)c1c(F)ccc2c1OB(O)C(NC(=O)[C@H](NC(=O)N1CCN(C(=O)c3ccc(O)c(O)c3Cl)C1=O)c1ccc(P(=O)(O)O)cc1)C2] | 3-((R)-2-(3-(2-chloro-3,4-dihydroxybenzoyl)-2- oxoimidazolidine-1-carboxamido)-2-(4- phosphonophenyl)acetamido)-7-fluoro-2- hydroxy-3,4-dihydro-2H- benzo[e][1,2]oxaborinine-8-carboxylic acid | 720.75 |
| 521 | 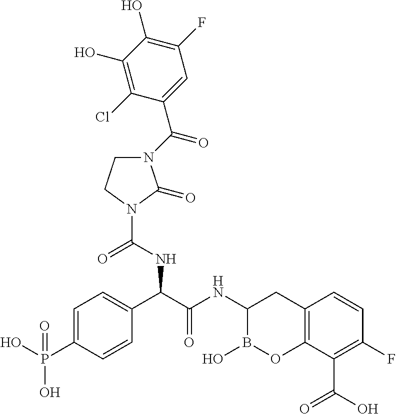 [SMILES: O=C(O)c1c(F)ccc2c1OB(O)C(NC(=O)[C@H](NC(=O)N1CCN(C(=O)c3cc(F)c(O)c(O)c3Cl)C1=O)c1ccc(P(=O)(O)O)cc1)C2] | 3-((R)-2-(3-(2-chloro-5-fluoro-3,4- dihydroxybenzoyl)-2-oxoimidazolidine-1- carboxamido)-2-(4- phosphonophenyl)acetamido)-7-fluoro-2- hydroxy-3,4-dihydro-2H- benzo[e][1,2]oxaborinine-8-carboxylic acid | 738.74 |
| 522 | 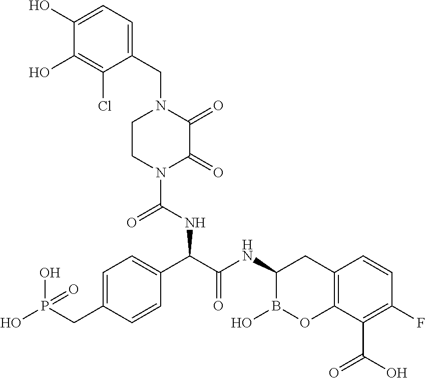 [SMILES: O=C(O)c1c(F)ccc2c1OB(O)[C@@H](NC(=O)[C@H](NC(=O)N1CCN(Cc3ccc(O)c(O)c3Cl)C(=O)C1=O)c1ccc(CP(=O)(O)O)cc1)C2] | (R)-3-((R)-2-(4-(2-chloro-3,4-dihydroxybenzyl)- 2,3-dioxopiperazine-1-carboxamido)-2-(4- (phosphonomethyl)phenyl)acetamido)-7-fluoro- 2-hydroxy-3,4-dihydro-2H- benzo[e][1,2]oxaborinine-8-carboxylic acid | 748.80 |
| 523 | 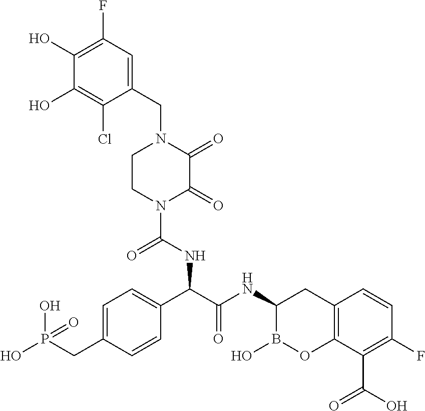 [SMILES: O=C(O)c1c(F)ccc2c1OB(O)[C@@H](NC(=O)[C@H](NC(=O)N1CCN(Cc3cc(F)c(O)c(O)c3Cl)C(=O)C1=O)c1ccc(CP(=O)(O)O)cc1)C2] | (R)-3-((R)-2-(4-(2-chloro-5-fluoro-3,4- dihydroxybenzyl)-2,3-dioxopiperazine-1- carboxamido)-2-(4- (phosphonomethyl)phenyl)acetamido)-7-fluoro- 2-hydroxy-3,4-dihydro-2H- benzo[e][1,2]oxaborinine-8-carboxylic acid | 766.79 |
| 524 | 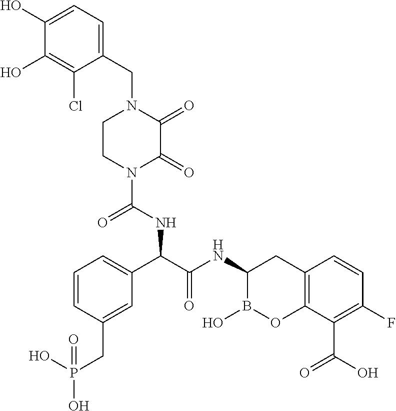 [SMILES: O=C(O)c1c(F)ccc2c1OB(O)[C@@H](NC(=O)[C@H](NC(=O)N1CCN(Cc3ccc(O)c(O)c3Cl)C(=O)C1=O)c1cccc(CP(=O)(O)O)c1)C2] | (R)-3-((R)-2-(4-(2-chloro-3,4-dihydroxybenzyl)- 2,3-dioxopiperazine-1-carboxamido)-2-(3- (phosphonomethyl)phenyl)acetamido)-7-fluoro- 2-hydroxy-3,4-dihydro-2H- benzo[e][1,2]oxaborinine-8-carboxylic acid | 748.80 |
| 525 | 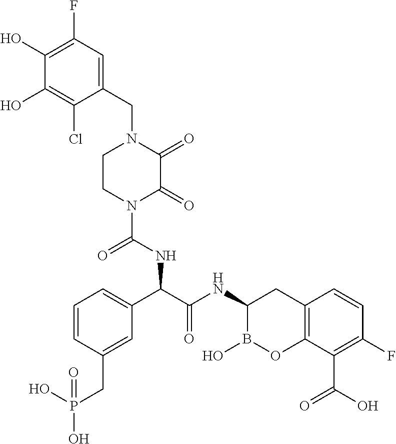 [SMILES: O=C(O)c1c(F)ccc2c1OB(O)[C@@H](NC(=O)[C@H](NC(=O)N1CCN(Cc3cc(F)c(O)c(O)c3Cl)C(=O)C1=O)c1cccc(CP(=O)(O)O)c1)C2] | (R)-3-((R)-2-(4-(2-chloro-5-fluoro-3,4- dihydroxybenzyl)-2,3-dioxopiperazine-1- carboxamido)-2-(3- (phosphonomethyl)phenyl)acetamido)-7-fluoro- 2-hydroxy-3,4-dihydro-2H- benzo[e][1,2]oxaborinine-8-carboxylic acid | 766.79 |

TABLE 3

| | | | | ESI-MS |
|---|---|---|---|---|
| | | | | (m/z) for |
| Ex. | Structure | Name | MW | [MH]⁺ |
| 200 | 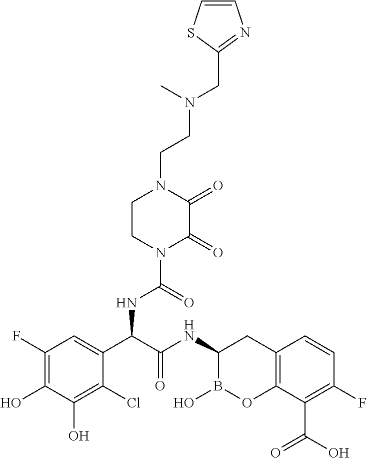 [SMILES: CN(CCN1CCN(C(=O)N[C@@H](C(=O)N[C@H]2Cc3ccc(F)c(C(=O)O)c3OB2O)c2cc(F)c(O)c(O)c2Cl)C(=O)C1=O)Cc1nccs1] | (R)-3-((R)-2-(2-chloro-5-fluoro- 3,4-dihydroxyphenyl)-2-(4-(2- (methyl(thiazol-2- ylmethyl)amino)ethyl)-2,3- dioxopiperazine-1- carboxamido)acetamido)-7- fluoro-2-hydroxy-3,4-dihydro- 2H-benzo[e][1,2]oxaborinine-8- carboxylic acid | 736.89 | 737 |
| 201 | 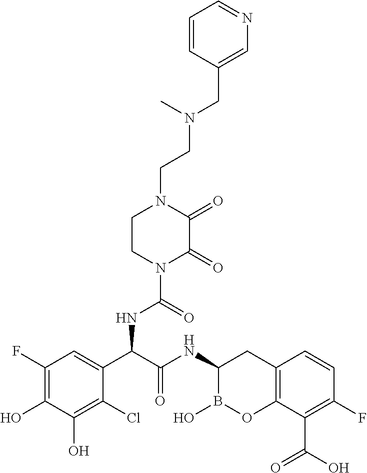 [SMILES: CN(CCN1CCN(C(=O)N[C@@H](C(=O)N[C@H]2Cc3ccc(F)c(C(=O)O)c3OB2O)c2cc(F)c(O)c(O)c2Cl)C(=O)C1=O)Cc1cccnc1] | (R)-3-((R)-2-(2-chloro-5-fluoro- 3,4-dihydroxyphenyl)-2-(4-(2- (methyl(pyridin-3- ylmethyl)amino)ethyl)-2,3- dioxopiperazine-1- carboxamido)acetamido)-7- fluoro-2-hydroxy-3,4-dihydro- 2H-benzo[e][1,2]oxaborinine-8- carboxylic acid | 730.87 | 731 |
| 202 | 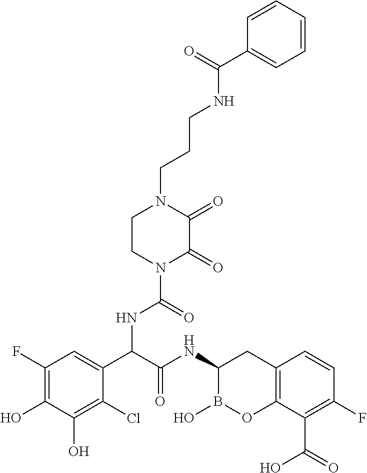 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccccc1] | (3R)-3-(2-(4-(3- benzamidopropyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 743.87 | 744 |
| 203 | 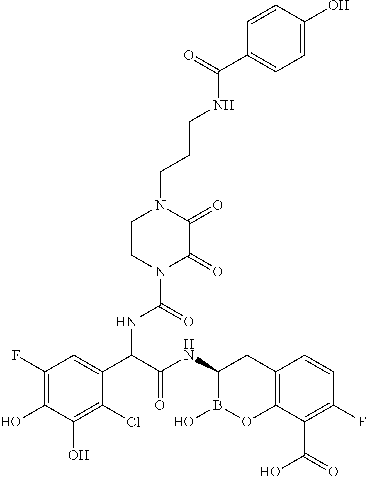 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(O)cc1] | (3R)-3-(2-(2-chloro-5-fluoro- 3,4-dihydroxyphenyl)-2-(4-(3- (4-hydroxybenzamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 759.86 | 760 |
| 204 | 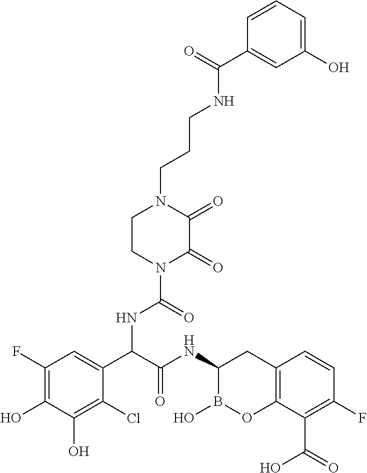 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cccc(O)c1] | (3R)-3-(2-(2-chloro-5-fluoro- 3,4-dihydroxyphenyl)-2-(4-(3- (3-hydroxybenzamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 759.86 | 760 |
| 205 | 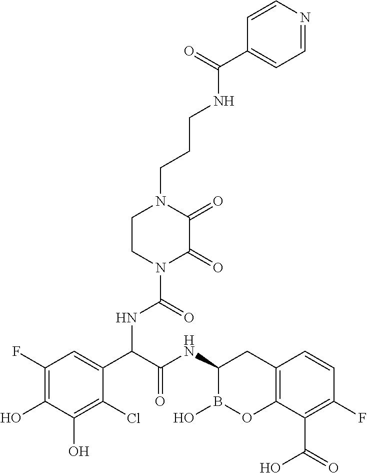 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccncc1] | (3R)-3-(2-(2-chloro-5-fluoro- 3,4-dihydroxyphenyl)-2-(4-(3- (isonicotinamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 744.85 | 745 |
| 206 | 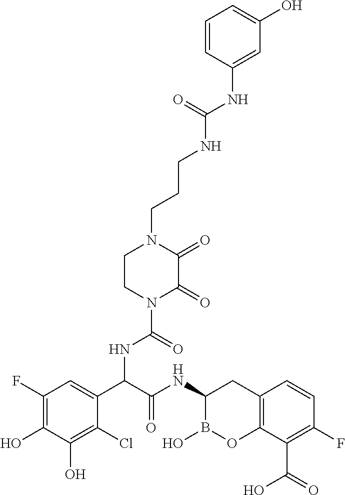 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)Nc1cccc(O)c1] | (3R)-3-(2-(2-chloro-5-fluoro- 3,4-dihydroxyphenyl)-2-(4-(3- (3-(3- hydroxyphenyl)ureido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 774.88 | 745 |
| 207 | 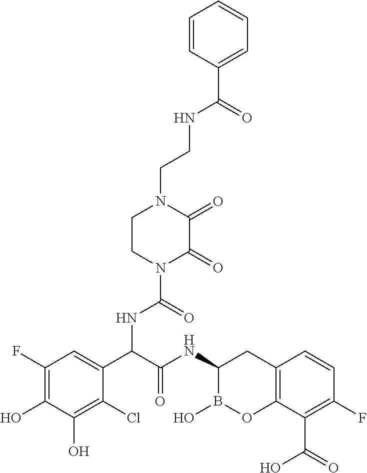 [SMILES: O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)c1ccccc1] | (3R)-3-(2-(4-(2- benzamidoethyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 729.84 | 730 |
| 208 | 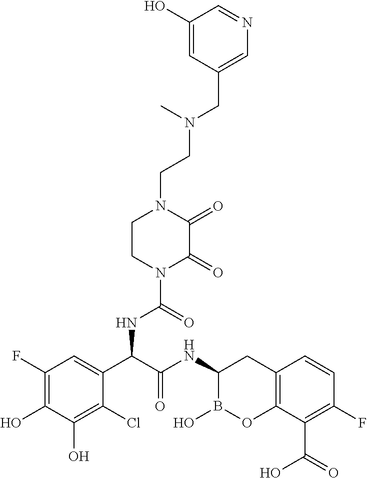 [SMILES: CN(CCN1CCN(C(=O)N[C@@H](C(=O)N[C@H]2Cc3ccc(F)c(C(=O)O)c3OB2O)c2cc(F)c(O)c(O)c2Cl)C(=O)C1=O)Cc1cncc(O)c1] | (R)-3-((R)-2-(2-chloro-5-fluoro- 3,4-dihydroxyphenyl)-2-(4-(2- (((5-hydroxypyridin-3- yl)methyl)(methyl)amino) ethyl)-2,3-dioxopiperazine-1- carboxamido)acetamido)-7- fluoro-2-hydroxy-3,4- dihydro- 2H-benzo[e][1,2]oxaborinine-8- carboxylic acid | 746.87 | 747 |
| 209 | 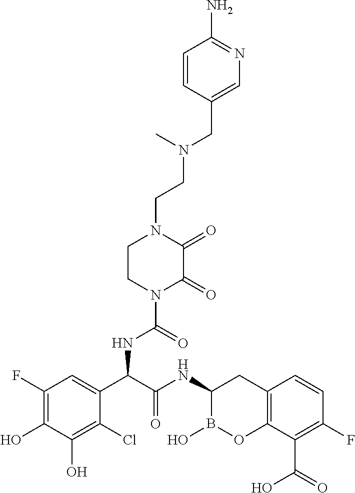 [SMILES: CN(CCN1CCN(C(=O)N[C@@H](C(=O)N[C@H]2Cc3ccc(F)c(C(=O)O)c3OB2O)c2cc(F)c(O)c(O)c2Cl)C(=O)C1=O)Cc1ccc(N)nc1] | (R)-3-((R)-2-(4-(2-(((6- aminopyridin-3- yl)methyl)(methyl)amino) ethyl)-2,3-dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 745.89 | 746 |
| 210 | 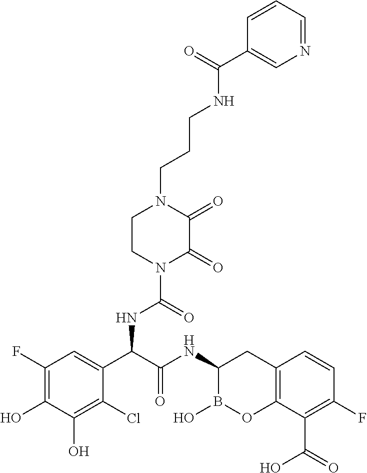 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1cccnc1] | (R)-3-((R)-2-(2-chloro-5-fluoro- 3,4-dihydroxyphenyl)-2-(4-(3- (nicotinamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 744.85 | 745 |
| 211 | 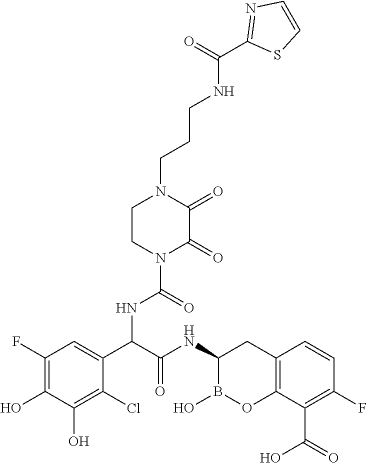 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1nccs1] | (3R)-3-(2-(2-chloro-5-fluoro- 3,4-dihydroxyphenyl)-2-(2,3- dioxo-4-(3-(thiazole-2- carboxamido)propyl)piperazine- 1-carboxamido)acetamido)-7- fluoro-2-hydroxy-3,4-dihydro- 2H-benzo[e][1,2]oxaborinine-8- carboxylic acid | 750.88 | 751 |
| 212 | 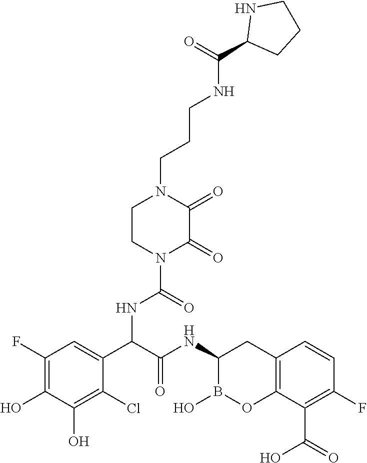 [SMILES: O=C(O)c1c(F)ccc2c1OB(O)[C@@H](NC(=O)C(NC(=O)N1CCN(CCCNC(=O)[C@@H]3CCCN3)C(=O)C1=O)c1cc(F)c(O)c(O)c1Cl)C2] | (3R)-3-(2-(2-chloro-5-fluoro- 3,4-dihydroxyphenyl)-2-(2,3- dioxo-4-(3-((S)-pyrrolidine-2- carboxamido)propyl)piperazine- 1-carboxamido)acetamido)-7- fluoro-2-hydroxy-3,4-dihydro- 2H-benzo[e][1,2]oxaborinine-8- carboxylic acid | 736.87 | 737 |
| 213 | 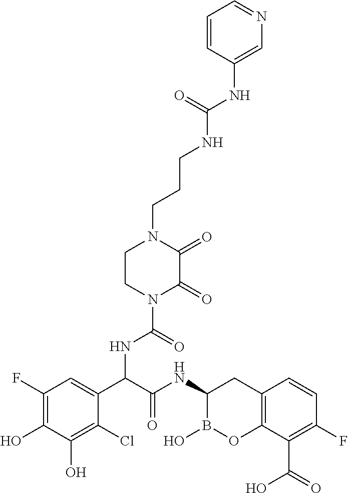 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)Nc1cccnc1] | (3R)-3-(2-(2-chloro-5-fluoro- 3,4-dihydroxyphenyl)-2-(2,3- dioxo-4-(3-(3-(pyridin-3- yl)ureido)propyl)piperazine-1- carboxamido)acetamido)-7- fluoro-2-hydroxy-3,4-dihydro- 2H-benzo[e][1,2]oxaborinine-8- carboxylic acid | 759.87 | 760 |
| 214 | 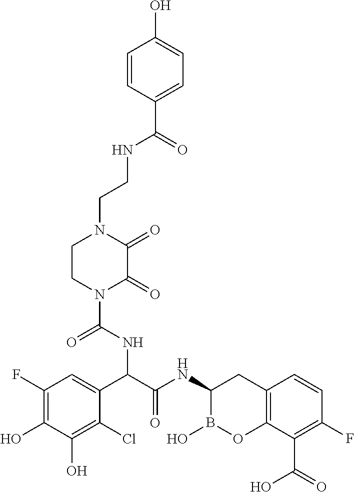 [SMILES: O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(O)cc1] | (3R)-3-(2-(2-chloro-5-fluoro- 3,4-dihydroxyphenyl)-2-(4-(2- (4-hydroxybenzamido)ethyl)- 2,3-dioxopiperazine-1- carboxamido)acetamido)-7- fluoro-2-hydroxy-3,4-dihydro- 2H-benzo[e][1,2]oxaborinine-8- carboxylic acid | 745.84 | 746 |
| 215 | 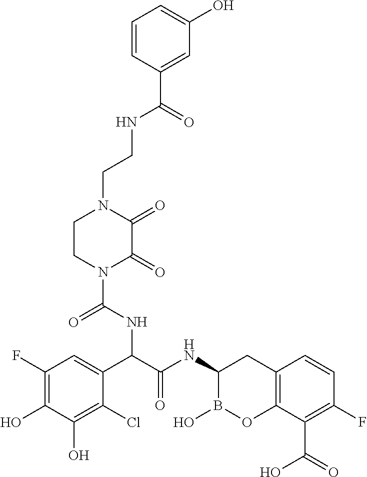 [SMILES: O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)c1cccc(O)c1] | (3R)-3-(2-(2-chloro-5-fluoro- 3,4-dihydroxyphenyl)-2-(4-(2- (3-hydroxybenzamido)ethyl)- 2,3-dioxopiperazine-1- carboxamido)acetamido)-7- fluoro-2-hydroxy-3,4-dihydro- 2H-benzo[e][1,2]oxaborinine-8- carboxylic acid | 745.84 | 746 |
| 216 | 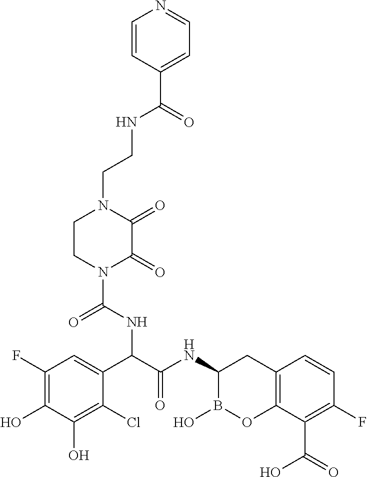 [SMILES: O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)c1ccncc1] | (3R)-3-(2-(2-chloro-5-fluoro- 3,4-dihydroxyphenyl)-2-(4-(2- (isonicotinamido)ethyl)-2,3- dioxopiperazine-1- carboxamido)acetamido)-7- fluoro-2-hydroxy-3,4-dihydro- 2H-benzo[e][1,2]oxaborinine-8- carboxylic acid | 730.83 | 731 |
| 217 | 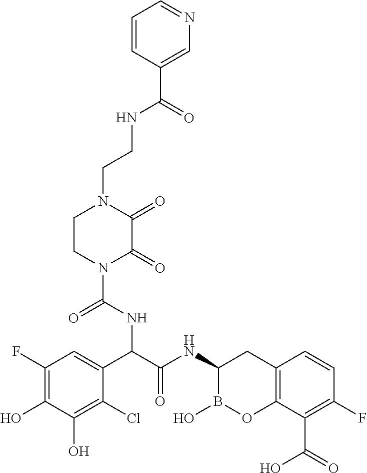 [SMILES: O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)c1cccnc1] | (3R)-3-(2-(2-chloro-5-fluoro- 3,4-dihydroxyphenyl)-2-(4-(2- (nicotinamido)ethyl)-2,3- dioxopiperazine-1- carboxamido)acetamido)-7- fluoro-2-hydroxy-3,4-dihydro- 2H-benzo[e][1,2]oxaborinine-8- carboxylic acid | 730.83 | 731 |
| 218 | 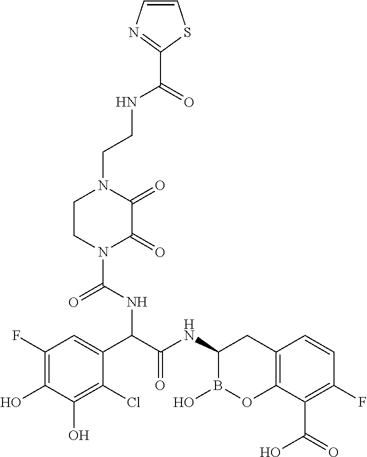 [SMILES: O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)c1nccs1] | (3R)-3-(2-(2-chloro-5-fluoro- 3,4-dihydroxyphenyl)-2-(2,3- dioxo-4-(2-(thiazole-2- carboxamido)ethyl)piperazine- 1-carboxamido)acetamido)-7- fluoro-2-hydroxy-3,4-dihydro- 2H-benzo[e][1,2]oxaborinine-8- carboxylic acid | 736.85 | 737 |
| 219 | 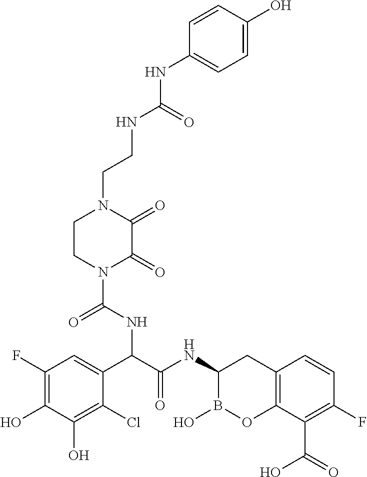 [SMILES: O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)Nc1ccc(O)cc1] | (3R)-3-(2-(2-chloro-5-fluoro- 3,4-dihydroxyphenyl)-2-(4-(2- (3-(4- hydroxyphenyl)ureido)ethyl)- 2,3-dioxopiperazine-1- carboxamido)acetamido)-7- fluoro-2-hydroxy-3,4-dihydro- 2H-benzo[e][1,2]oxaborinine-8- carboxylic acid | 760.85 | 761 |
| 220 | 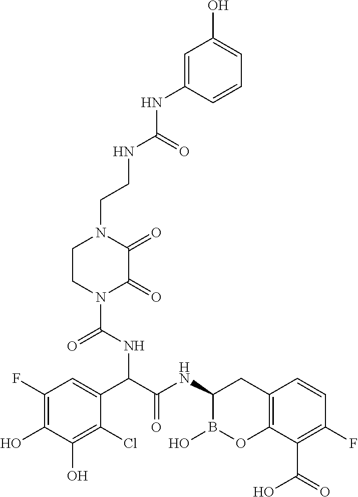 [SMILES: O=C(NCCN1CCN(C(=O)NC(C(=O)N[C@H]2Cc3ccc(F)c(C(=O)O)c3OB2O)c2cc(F)c(O)c(O)c2Cl)C(=O)C1=O)Nc1cccc(O)c1] | (3R)-3-(2-(2-chloro-5-fluoro- 3,4-dihydroxyphenyl)-2-(4-(2- (3-(3- hydroxyphenyl)ureido)ethyl)- 2,3-dioxopiperazine-1- carboxamido)acetamido)-7- fluoro-2-hydroxy-3,4-dihydro- 2H-benzo[e][1,2]oxaborinine-8- carboxylic acid | 760.85 | 761 |
| 221 | 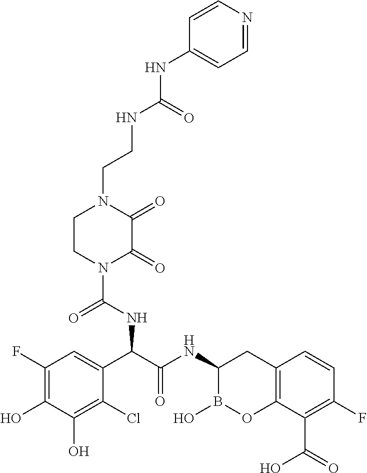 [SMILES: O=C(NCCN1CCN(C(=O)N[C@@H](C(=O)N[C@H]2Cc3ccc(F)c(C(=O)O)c3OB2O)c2cc(F)c(O)c(O)c2Cl)C(=O)C1=O)Nc1ccncc1] | (R)-3-((R)-2-(2-chloro-5-fluoro- 3,4-dihydroxyphenyl)-2-(2,3- dioxo-4-(2-(3-(pyridin-4- yl)ureido)ethyl)piperazine-1- carboxamido)acetamido)-7- fluoro-2-hydroxy-3,4-dihydro- 2H-benzo[e][1,2]oxaborinine-8- carboxylic acid | 745.84 | 746 |
| 222 | 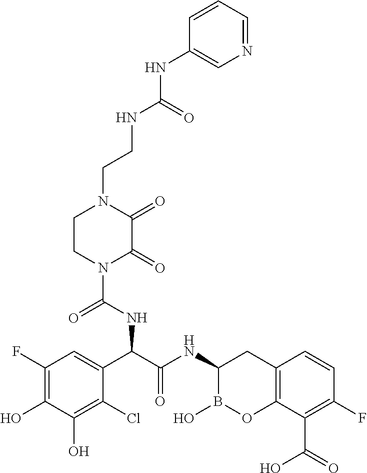 [SMILES: O=C(NCCN1CCN(C(=O)N[C@@H](C(=O)N[C@H]2Cc3ccc(F)c(C(=O)O)c3OB2O)c2cc(F)c(O)c(O)c2Cl)C(=O)C1=O)Nc1cccnc1] | (R)-3-((R)-2-(2-chloro-5-fluoro- 3,4-dihydroxyphenyl)-2-(2,3- dioxo-4-(2-(3-(pyridin-3- yl)ureido)ethyl)piperazine-1- carboxamido)acetamido)-7- fluoro-2-hydroxy-3,4-dihydro- 2H-benzo[e][1,2]oxaborinine-8- carboxylic acid | 745.84 | 746 |
| 223 | 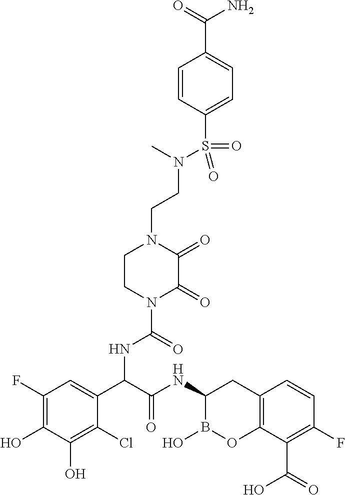 [SMILES: CN(CCN1CCN(C(=O)NC(C(=O)N[C@H]2Cc3ccc(F)c(C(=O)O)c3OB2O)c2cc(F)c(O)c(O)c2Cl)C(=O)C1=O)S(=O)(=O)c1ccc(C(N)=O)cc1] | (3R)-3-(2-(4-(2-((4-carbamoyl- N- methylphenyl)sulfonamido) ethyl)-2,3-dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 822.94 | 823 |
| 224 | 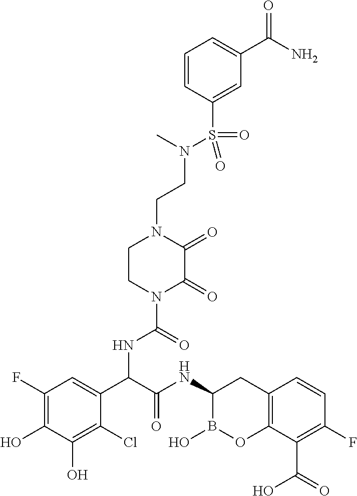 [SMILES: CN(CCN1CCN(C(=O)NC(C(=O)N[C@H]2Cc3ccc(F)c(C(=O)O)c3OB2O)c2cc(F)c(O)c(O)c2Cl)C(=O)C1=O)S(=O)(=O)c1cccc(C(N)=O)c1] | (3R)-3-(2-(4-(2-((3-carbamoyl- N- methylphenyl)sulfonamido) ethyl)-2,3-dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 822.94 | 823 |
| 225 | 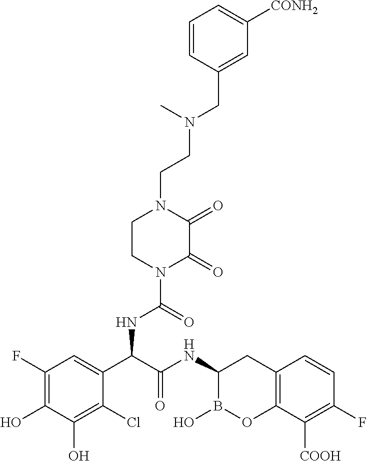 [SMILES: CN(CCN1CCN(C(=O)N[C@@H](C(=O)N[C@H]2Cc3ccc(F)c(C(=O)O)c3OB2O)c2cc(F)c(O)c(O)c2Cl)C(=O)C1=O)Cc1cccc(C(N)=O)c1] | (R)-3-((R)-2-(4-(2-((3- carbamoylbenzyl)(methyl) amino)ethyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 772.91 | 773 |
| 226 | 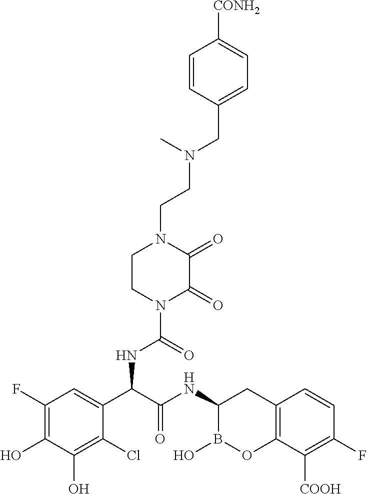 [SMILES: CN(CCN1CCN(C(=O)N[C@@H](C(=O)N[C@H]2Cc3ccc(F)c(C(=O)O)c3OB2O)c2cc(F)c(O)c(O)c2Cl)C(=O)C1=O)Cc1ccc(C(N)=O)cc1] | (R)-3-((R)-2-(4-(2-((4- carbamoylbenzyl)(methyl) amino)ethyl)-3,4- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 772.91 | 773 |
| 227 | 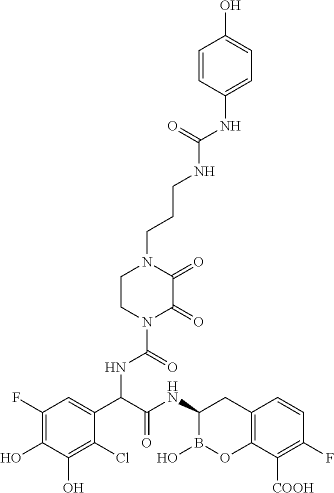 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)Nc1ccc(O)cc1] | (3R)-3-(2-(2-chloro-5-fluoro- 3,4-dihydroxyphenyl)-2-(4-(3- (3-(4- hydroxyphenyl)ureido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 774.88 | 775 |
| 228 | 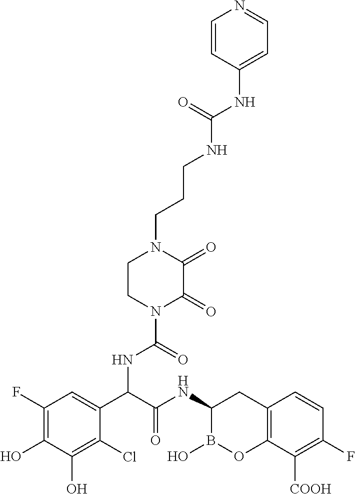 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)Nc1ccncc1] | (3R)-3-(2-(2-chloro-5-fluoro- 3,4-dihydroxyphenyl)-2-(2,3- dioxo-4-(3-(3-(pyridin-4- yl)ureido)propyl)piperazine-1- carboxamido)acetamido)-7- fluoro-2-hydroxy-3,4-dihydro- 2H-benzo[e][1,2]oxaborinine-8- carboxylic acid | 759.87 | 760 |
| 229 | 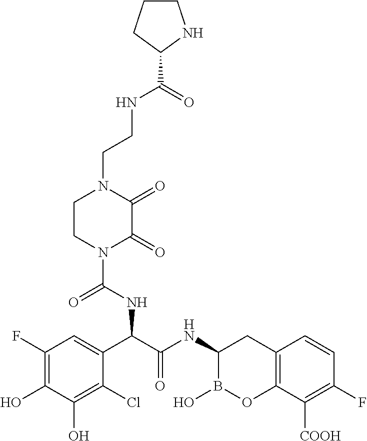 [SMILES: O=C(O)c1c(F)ccc2c1OB(O)[C@@H](NC(=O)[C@H](NC(=O)N1CCN(CCNC(=O)[C@@H]3CCCN3)C(=O)C1=O)c1cc(F)c(O)c(O)c1Cl)C2] | (R)-3-((R)-2-(2-chloro-5-fluoro- 3,4-dihydroxyphenyl)-2-(2,3- dioxo-4-(2-((S)-pyrrolidine-2- carboxamido)ethyl)piperazine- 1-carboxamido)acetamido)-7- fluoro-2-hydroxy-3,4-dihydro- 2H-benzo[e][1,2]oxaborinine-8- carboxylic acid | 722.85 | 723 |
| 230 | 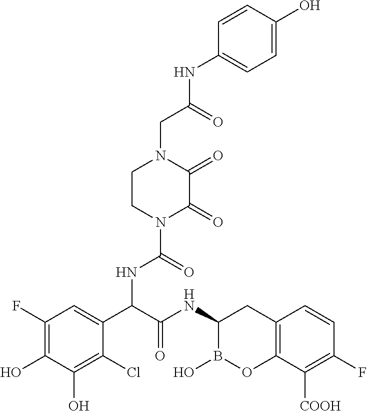 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)Nc1ccc(O)cc1] | (3R)-3-(2-(2-chloro-5-fluoro- 3,4-dihydroxyphenyl)-2-(4-(2- ((4-hydroxyphenyl)amino)-2- oxoethyl)-2,3-dioxopiperazine- 1-carboxamido)acetamido)-7- fluoro-2-hydroxy-3,4-dihydro- 2H-benzo[e][1,2]oxaborinine-8- carboxylic acid | 731.81 | 732 |
| 231 | 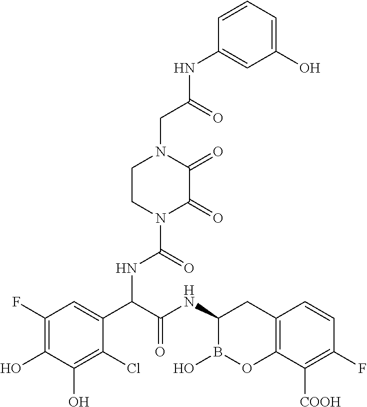 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)Nc1cccc(O)c1] | (3R)-3-(2-(2-chloro-5-fluoro- 3,4-dihydroxyphenyl)-2-(4-(2- ((3-hydroxyphenyl)amino)-2- oxoethyl)-2,3-dioxopiperazine- 1-carboxamido)acetamido)-7- fluoro-2-hydroxy-3,4-dihydro- 2H-benzo[e][1,2]oxaborinine-8- carboxylic acid | 731.81 | 732 |
| 232 | 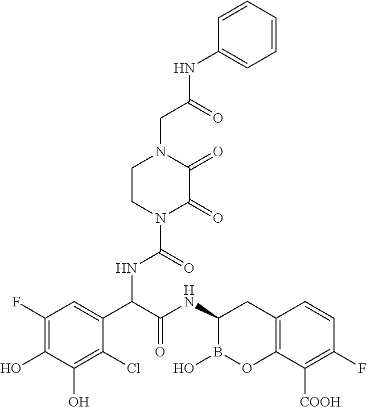 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)Nc1ccccc1] | (3R)-3-(2-(2-chloro-5-fluoro- 3,4-dihydroxyphenyl)-2-(2,3- dioxo-4-(2-oxo-2- (phenylamino)ethyl)piperazine- 1-carboxamido)acetamido)-7- fluoro-2-hydroxy-3,4-dihydro- 2H-benzo[e][1,2]oxaborinine-8- carboxylic acid | 715.81 | 716 |
| 233 | 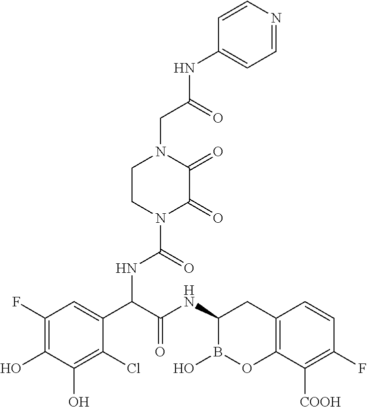 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)Nc1ccncc1] | (3R)-3-(2-(2-chloro-5-fluoro- 3,4-dihydroxyphenyl)-2-(2,3- dioxo-4-(2-oxo-2-(pyridin-4- ylamino)ethyl)piperazine-1- carboxamido)acetamido)-7- fluoro-2-hydroxy-3,4-dihydro- 2H-benzo[e][1,2]oxaborinine-8- carboxylic acid | 716.80 | 717 |
| 234 | 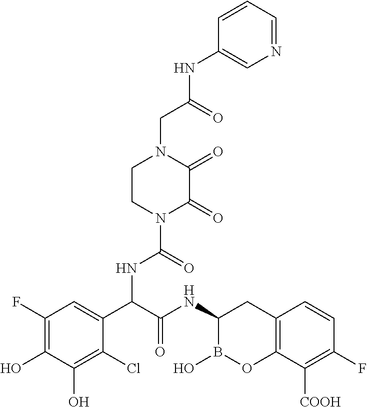 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)Nc1cccnc1] | (3R)-3-(2-(2-chloro-5-fluoro- 3,4-dihydroxyphenyl)-2-(2,3- dioxo-4-(2-oxo-2-(pyridin-3- ylamino)ethyl)piperazine-1- carboxamido)acetamido)-7- fluoro-2-hydroxy-3,4-dihydro- 2H-benzo[e][1,2]oxaborinine-8- carboxylic acid | 716.80 | 717 |
| 235 | 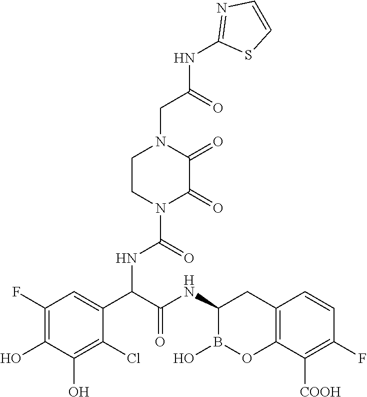 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)Nc1nccs1] | (3R)-3-(2-(2-chloro-5-fluoro- 3,4-dihydroxyphenyl)-2-(2,3- dioxo-4-(2-oxo-2-(thiazol-2- ylamino)ethyl)piperazine-1- carboxamido)acetamido)-7- fluoro-2-hydroxy-3,4-dihydro- 2H-benzo[e][1,2]oxaborinine-8- carboxylic acid | 722.82 | 723 |
| 236 | 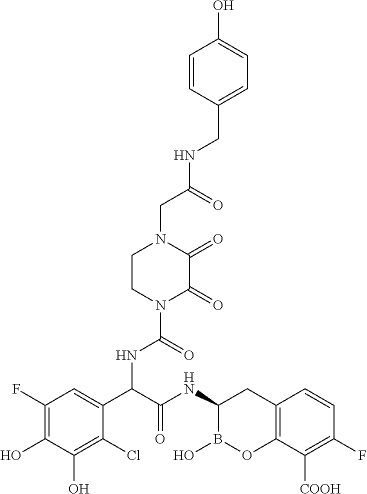 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)NCc1ccc(O)cc1] | (3R)-3-(2-(2-chloro-5-fluoro- 3,4-dihydroxyphenyl)-2-(4-(2- ((4-hydroxybenzyl)amino)-2- oxoethyl)-2,3-dioxopiperazine- 1-carboxamido)acetamido)-7- fluoro-2-hydroxy-3,4-dihydro- 2H-benzo[e][1,2]oxaborinine-8- carboxylic acid | 745.84 | 746 |
| 237 | 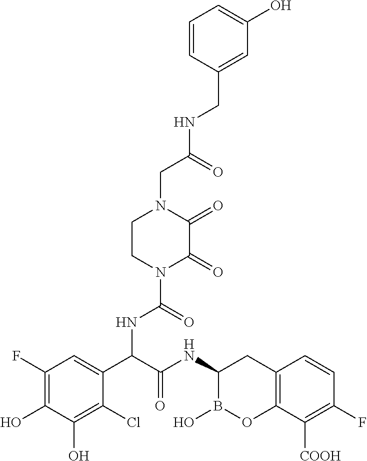 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)NCc1cccc(O)c1] | (3R)-3-(2-(2-chloro-5-fluoro- 3,4-dihydroxyphenyl)-2-(4-(2- ((3-hydroxybenzyl)amino)-2- oxoethyl)-2,3-dioxopiperazine- 1-carboxamido)acetamido)-7- fluoro-2-hydroxy-3,4-dihydro- 2H-benzo[e][1,2]oxaborinine-8- carboxylic acid | 745.84 | 746 |
| 238 | 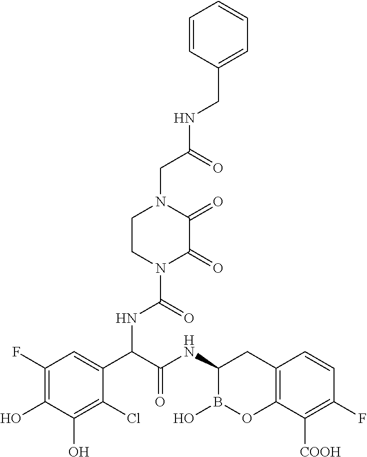 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)NCc1ccccc1] | (3R)-3-(2-(4-(2-(benzylamino)- 2-oxoethyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 729.84 | 730 |
| 239 | 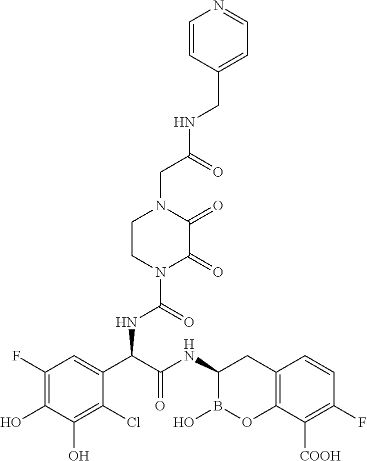 [SMILES: O=C(CN1CCN(C(=O)N[C@@H](C(=O)N[C@H]2Cc3ccc(F)c(C(=O)O)c3OB2O)c2cc(F)c(O)c(O)c2Cl)C(=O)C1=O)NCc1ccncc1] | (R)-3-((R)-2-(2-chloro-5-fluoro- 3,4-dihydroxyphenyl)-2-(2,3- dioxo-4-(2-oxo-2-((pyridin-4- ylmethyl)amino)ethyl) piperazine-1-carboxamido) acetamido)-7-fluoro-2- hydroxy-3,4-dihydro- 2H-benzo[e][1,2]oxaborinine-8- carboxylic acid | 730.83 | 731 |
| 240 | 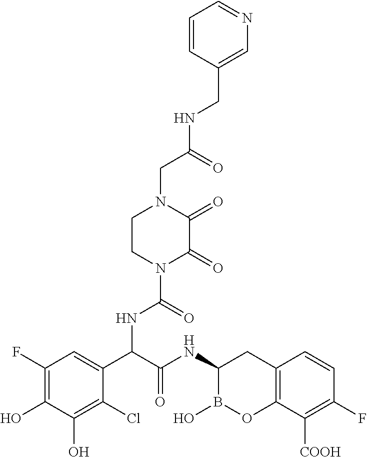 [SMILES: O=C(CN1CCN(C(=O)NC(C(=O)N[C@H]2Cc3ccc(F)c(C(=O)O)c3OB2O)c2cc(F)c(O)c(O)c2Cl)C(=O)C1=O)NCc1cccnc1] | (3R)-3-(2-(2-chloro-5-fluoro- 3,4-dihydroxyphenyl)-2-(2,3- dioxo-4-(2-oxo-2-((pyridin-3- ylmethyl)amino)ethyl) piperazine-1-carboxamido) acetamido)-7-fluoro-2- hydroxy-3,4-dihydro- 2H-benzo[e][1,2]oxaborinine-8- carboxylic acid | 730.83 | 731 |
| 241 | 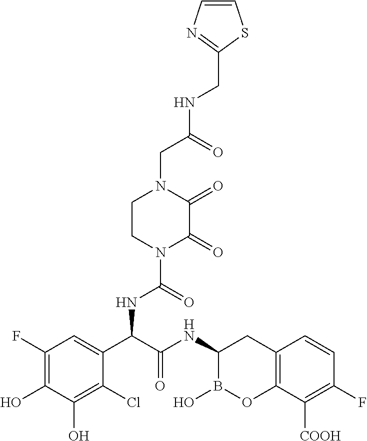 [SMILES: O=C(CN1CCN(C(=O)N[C@@H](C(=O)N[C@H]2Cc3ccc(F)c(C(=O)O)c3OB2O)c2cc(F)c(O)c(O)c2Cl)C(=O)C1=O)NCc1nccs1] | (R)-3-((R)-2-(2-chloro-5-fluoro- 3,4-dihydroxyphenyl)-2-(2,3- dioxo-4-(2-oxo-2-((thiazol-2- ylmethyl)amino)ethyl) piperazine-1-carboxamido) acetamido)-7-fluoro-2- hydroxy-3,4-dihydro- 2H-benzo[e][1,2]oxaborinine-8- carboxylic acid | 736.85 | 737 |
| 242 | 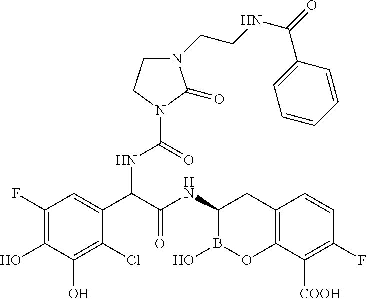 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)c1ccccc1] | (3R)-3-(2-(3-(2- benzamidoethyl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 701.83 | 702 |
| 243 | 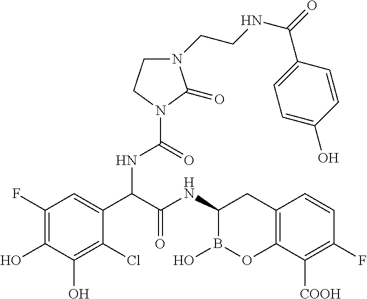 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)c1ccc(O)cc1] | (3R)-3-(2-(2-chloro-5-fluoro- 3,4-dihydroxyphenyl)-2-(3-(2- (4-hydroxybenzamido)ethyl)-2- oxoimidazolidine-1- carboxamido)acetamido)-7- fluoro-2-hydroxy-3,4-dihydro- 2H-benzo[e][1,2]oxaborinine-8- carboxylic acid | 717.83 | 718 |
| 244 | 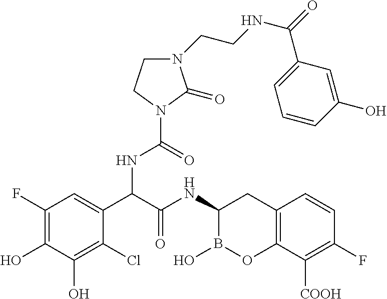 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)c1cccc(O)c1] | (3R)-3-(2-(2-chloro-5-fluoro- 3,4-dihydroxyphenyl)-2-(3-(2- (3-hydroxybenzamido)ethyl)-2- oxoimidazolidine-1- carboxamido)acetamido)-7- fluoro-2-hydroxy-3,4-dihydro- 2H-benzo[e][1,2]oxaborinine-8- carboxylic acid | 717.83 | 718 |
| 245 | 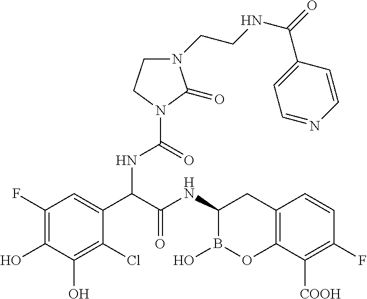 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)c1ccncc1] | (3R)-3-(2-(2-chloro-5-fluoro- 3,4-dihydroxyphenyl)-2-(3-(2- (isonicotinamido)ethyl)-2- oxoimidazolidine-1- carboxamido)acetamido)-7- fluoro-2-hydroxy-3,4-dihydro- 2H-benzo[e][1,2]oxaborinine-8- carboxylic acid | 702.82 | 703 |
| 246 | 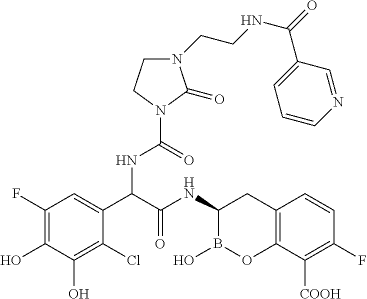 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)c1cccnc1] | (3R)-3-(2-(2-chloro-5-fluoro- 3,4-dihydroxyphenyl)-2-(3-(2- (nicotinamido)ethyl)-2- oxoimidazolidine-1- carboxamido)acetamido)-7- fluoro-2-hydroxy-3,4-dihydro- 2H-benzo[e][1,2]oxaborinine-8- carboxylic acid | 702.82 | 703 |
| 247 | 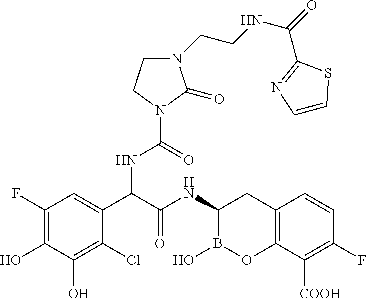 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)c1nccs1] | (3R)-3-(2-(2-chloro-5-fluoro- 3,4-dihydroxyphenyl)-2-(2-oxo- 3-(2-(thiazole-2- carboxamido)ethyl) imidazolidine-1-carboxamido) acetamido)-7-fluoro-2- hydroxy-3,4-dihydro- 2H-benzo[e][1,2]oxaborinine-8- carboxylic acid | 708.84 | 709 |
| 248 | 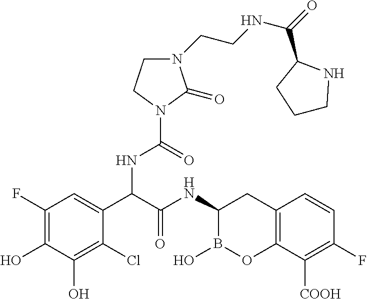 [SMILES: O=C(O)c1c(F)ccc2c1OB(O)[C@@H](NC(=O)C(NC(=O)N1CCN(CCNC(=O)[C@@H]3CCCN3)C1=O)c1cc(F)c(O)c(O)c1Cl)C2] | (3R)-3-(2-(2-chloro-5-fluoro- 3,4-dihydroxyphenyl)-2-(2-oxo- 3-(2-((S)-pyrrolidine-2- carboxamido)ethyl) imidazolidine-1-carboxamido) acetamido)-7-fluoro-2- hydroxy-3,4-dihydro- 2H-benzo[e][1,2]oxaborinine-8- carboxylic acid | 694.84 | 695 |
| 249 | 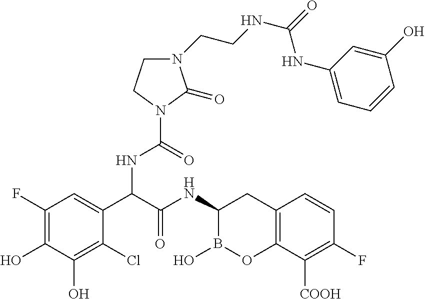 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)Nc1cccc(O)c1] | (3R)-3-(2-(2-chloro-5-fluoro- 3,4-dihydroxyphenyl)-2-(3-(2- (3-(3- hydroxyphenyl)ureido)ethyl)-2- oxoimidazolidine-1- carboxamido)acetamido)-7- fluoro-2-hydroxy-3,4-dihydro- 2H-benzo[e][1,2]oxaborinine-8- carboxylic acid | 732.84 | 733 |
| 250 | 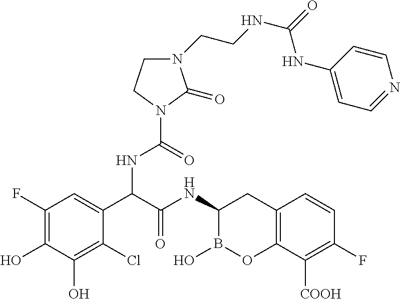 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)Nc1ccncc1] | (3R)-3-(2-(2-chloro-5-fluoro- 3,4-dihydroxyphenyl)-2-(2-oxo- 3-(2-(3-(pyridin-4- yl)ureido)ethyl)imidazolidine- 1-carboxamido)acetamido)-7- fluoro-2-hydroxy-3,4-dihydro- 2H-benzo[e][1,2]oxaborinine-8- carboxylic acid | 717.83 | 718 |
| 251 | 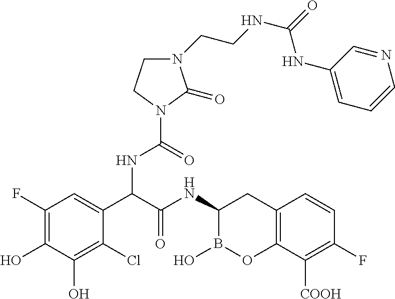 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)Nc1cccnc1] | (3R)-3-(2-(2-chloro-5-fluoro- 3,4-dihydroxyphenyl)-2-(2-oxo- 3-(2-(3-(pyridin-3- yl)ureido)ethyl)imidazolidine- 1-carboxamido)acetamido)-7- fluoro-2-hydroxy-3,4-dihydro- 2H-benzo[e][1,2]oxaborinine-8- carboxylic acid | 717.83 | 718 |
| 252 | 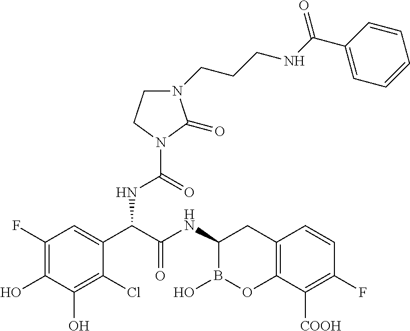 [SMILES: O=C(NCCCN1CCN(C(=O)N[C@H](C(=O)N[C@H]2Cc3ccc(F)c(C(=O)O)c3OB2O)c2cc(F)c(O)c(O)c2Cl)C1=O)c1ccccc1] | (R)-3-((S)-2-(3-(3- benzamidopropyl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 715.86 | 716 |
| 253 | 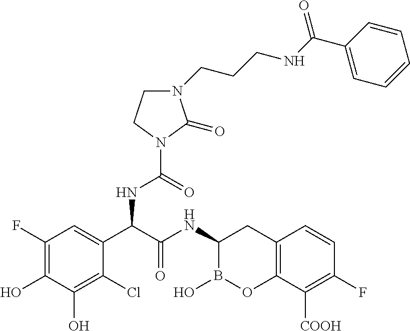 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C1=O)c1ccccc1] | (R)-3-((R)-2-(3-(3- benzamidopropyl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 715.86 | 716 |
| 254 | 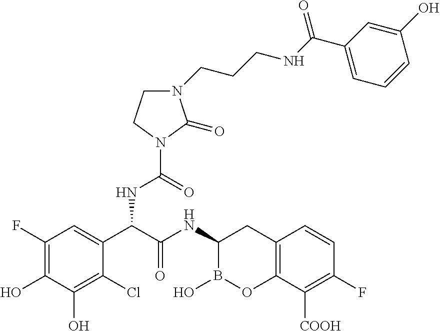 [SMILES: O=C(NCCCN1CCN(C(=O)N[C@H](C(=O)N[C@H]2Cc3ccc(F)c(C(=O)O)c3OB2O)c2cc(F)c(O)c(O)c2Cl)C1=O)c1cccc(O)c1] | (R)-3-((S)-2-(2-chloro-5-fluoro- 3,4-dihydroxyphenyl)-2-(3-(3- (3-hydroxybenzamido)propyl)- 2-oxoimidazolidine-1- carboxamido)acetamido)-7- fluoro-2-hydroxy-3,4-dihydro- 2H-benzo[e][1,2]oxaborinine-8- carboxylic acid | 731.85 | 732 |
| 255 | 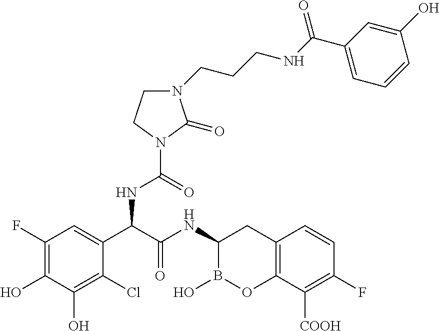 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C1=O)c1cccc(O)c1] | (R)-3-((R)-2-(2-chloro-5-fluoro- 3,4-dihydroxyphenyl)-2-(3-(3- (3-hydroxybenzamido)propyl)- 2-oxoimidazolidine-1- carboxamido)acetamido)-7- fluoro-2-hydroxy-3,4-dihydro- 2H-benzo[e][1,2]oxaborinine-8- carboxylic acid | 731.85 | 732 |
| 256 | 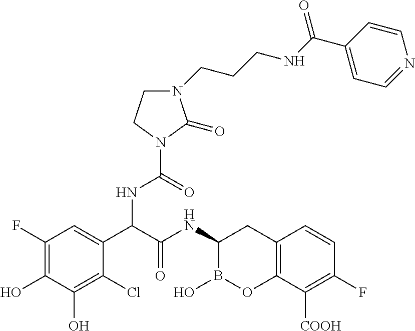 [SMILES: O=C(NCCCN1CCN(C(=O)NC(C(=O)N[C@H]2Cc3ccc(F)c(C(=O)O)c3OB2O)c2cc(F)c(O)c(O)c2Cl)C1=O)c1ccncc1] | (3R)-3-(2-(2-chloro-5-fluoro- 3,4-dihydroxyphenyl)-2-(3-(3- (isonicotinamido)propyl)-2- oxoimidazolidine-1- carboxamido)acetamido)-7- fluoro-2-hydroxy-3,4-dihydro- 2H-benzo[e][1,2]oxaborinine-8- carboxylic acid | 716.84 | 717 |
| 257 | 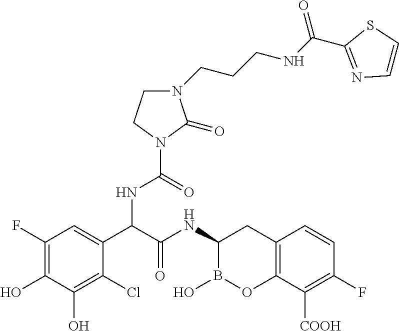 [SMILES: O=C(NCCCN1CCN(C(=O)NC(C(=O)N[C@H]2Cc3ccc(F)c(C(=O)O)c3OB2O)c2cc(F)c(O)c(O)c2Cl)C1=O)c1nccs1] | (3R)-3-(2-(2-chloro-5-fluoro- 3,4-dihydroxyphenyl)-2-(2-oxo- 3-(3-(thiazole-2- carboxamido)propyl) imidazolidine-1-carboxamido) acetamido)-7-fluoro-2- hydroxy-3,4-dihydro- 2H-benzo[e][1,2]oxaborinine-8- carboxylic acid | 722.87 | 723 |
| 258 | 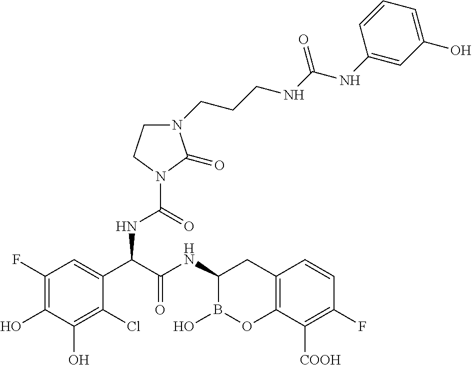 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C1=O)Nc1cccc(O)c1] | (R)-3-((R)-2-(2-chloro-5-fluoro- 3,4-dihydroxyphenyl)-2-(3-(3- (3-(3- hydroxyphenyl)ureido)propyl)- 2-oxoimidazolidine-1- carboxamido)acetamido)-7- fluoro-2-hydroxy-3,4-dihydro- 2H-benzo[e][1,2]oxaborinine-8- carboxylic acid | 746.87 | 747 |
| 259 | 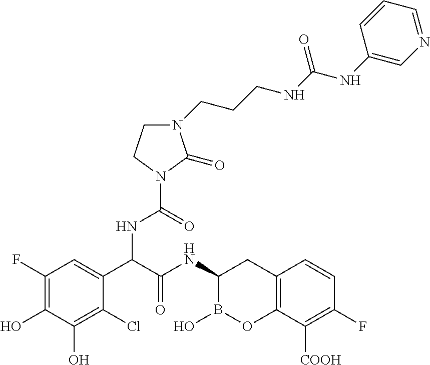 [SMILES: O=C(NCCCN1CCN(C(=O)NC(C(=O)N[C@H]2Cc3ccc(F)c(C(=O)O)c3OB2O)c2cc(F)c(O)c(O)c2Cl)C1=O)Nc1cccnc1] | (3R)-3-(2-(2-chloro-5-fluoro- 3,4-dihydroxyphenyl)-2-(2-oxo- 3-(3-(3-(pyridin-3- yl)ureido)propyl) imidazolidine-1-carboxamido) acetamido)-7-fluoro-2- hydroxy-3,4-dihydro- 2H-benzo[e][1,2]oxaborinine-8- carboxylic acid | 731.89 | 732 |
| 260 | 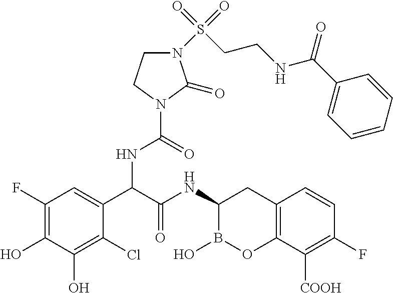 [SMILES: O=C(NCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1ccccc1] | (3R)-3-(2-(3-((2- benzamidoethyl)sulfonyl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 765.89 | 766 |
| 261 | 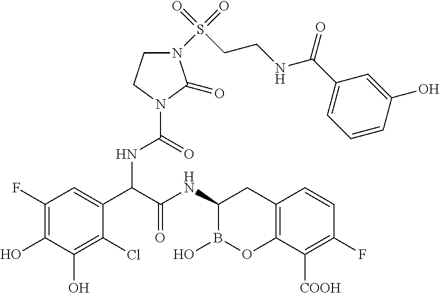 [SMILES: O=C(NCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1cccc(O)c1] | (3R)-3-(2-(2-chloro-5-fluoro- 3,4-dihydroxyphenyl)-2-(3-((2- (3- hydroxybenzamido)ethyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 781.89 | 782 |
| 262 | 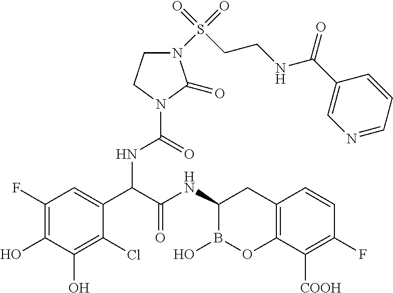 [SMILES: O=C(NCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1cccnc1] | (3R)-3-(2-(2-chloro-5-fluoro- 3,4-dihydroxyphenyl)-2-(3-((2- (nicotinamido)ethyl)sulfonyl)- 2-oxoimidazolidine-1- carboxamido)acetamido)-7- fluoro-2-hydroxy-3,4-dihydro- 2H-benzo[e][1,2]oxaborinine-8- carboxylic acid | 766.87 | 767 |
| 263 | 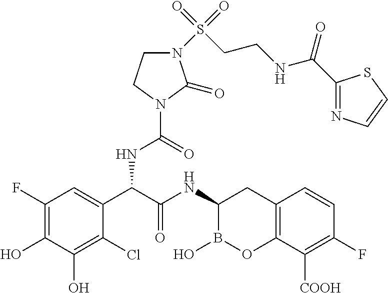 [SMILES: O=C(NCCS(=O)(=O)N1CCN(C(=O)N[C@H](C(=O)N[C@H]2Cc3ccc(F)c(C(=O)O)c3OB2O)c2cc(F)c(O)c(O)c2Cl)C1=O)c1nccs1] | (R)-3-((S)-2-(2-chloro-5-fluoro- 3,4-dihydroxyphenyl)-2-(2- oxo-3-((2-(thiazole-2- carboxamido)ethyl)sulfonyl) imidazolidine-1- carboxamido)acetamido)-7- fluoro-2-hydroxy-3,4-dihydro- 2H-benzo[e][1,2]oxaborinine-8- carboxylic acid | 772.90 | 773 |
| 264 | 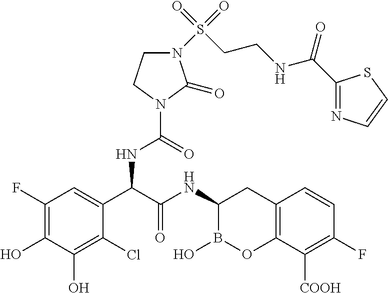 [SMILES: O=C(NCCS(=O)(=O)N1CCN(C(=O)N[C@@H](C(=O)N[C@H]2Cc3ccc(F)c(C(=O)O)c3OB2O)c2cc(F)c(O)c(O)c2Cl)C1=O)c1nccs1] | (R)-3-((R)-2-(2-chloro-5-fluoro- 3,4-dihydroxyphenyl)-2-(2- oxo-3-((2-(thiazole-2- carboxamido)ethyl)sulfonyl) imidazolidine-1- carboxamido)acetamido)-7- fluoro-2-hydroxy-3,4-dihydro- 2H-benzo[e][1,2]oxaborinine-8- carboxylic acid | 772.90 | 773 |
| 265 | 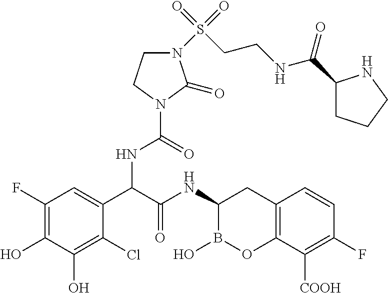 [SMILES: O=C(O)c1c(F)ccc2c1OB(O)[C@@H](NC(=O)C(NC(=O)N1CCN(S(=O)(=O)CCNC(=O)[C@@H]3CCCN3)C1=O)c1cc(F)c(O)c(O)c1Cl)C2] | (3R)-3-(2-(2-chloro-5-fluoro- 3,4-dihydroxyphenyl)-2-(2- oxo-3-((2-((S)-pyrrolidine-2- carboxamido)ethyl)sulfonyl) imidazolidine-1- carboxamido)acetamido)-7- fluoro-2-hydroxy-3,4-dihydro- 2H-benzo[e][1,2]oxaborinine-8- carboxylic acid | 758.90 | 759 |
| 266 | 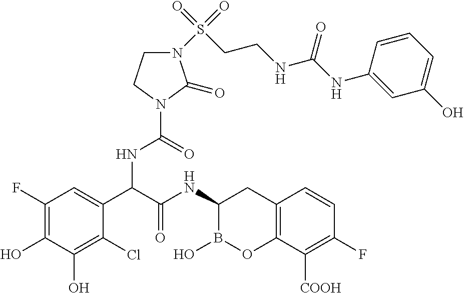 [SMILES: O=C(NCCS(=O)(=O)N1CCN(C(=O)NC(C(=O)N[C@H]2Cc3ccc(F)c(C(=O)O)c3OB2O)c2cc(F)c(O)c(O)c2Cl)C1=O)Nc1cccc(O)c1] | (3R)-3-(2-(2-chloro-5-fluoro- 3,4-dihydroxyphenyl)-2-(3-((2- (3-(3- hydroxyphenyl)ureido)ethyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 796.90 | 797 |
| 267 | 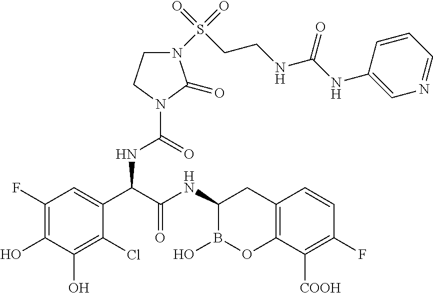 [SMILES: O=C(NCCS(=O)(=O)N1CCN(C(=O)N[C@@H](C(=O)N[C@H]2Cc3ccc(F)c(C(=O)O)c3OB2O)c2cc(F)c(O)c(O)c2Cl)C1=O)Nc1cccnc1] | (R)-3-((R)-2-(2-chloro-5-fluoro- 3,4-dihydroxyphenyl)-2-(2-oxo- 3-((2-(3-(pyridin-3- yl)ureido)ethyl)sulfonyl) imidazolidine-1- carboxamido)acetamido)-7- fluoro-2-hydroxy-3,4-dihydro- 2H-benzo[e][1,2]oxaborinine-8- carboxylic acid | 781.89 | 782 |
| 268 | 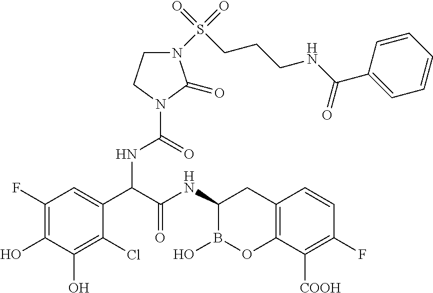 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1ccccc1] | (3R)-3-(2-(3-((3- benzamidopropyl)sulfonyl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 779.913 | 780 |
| 269 | 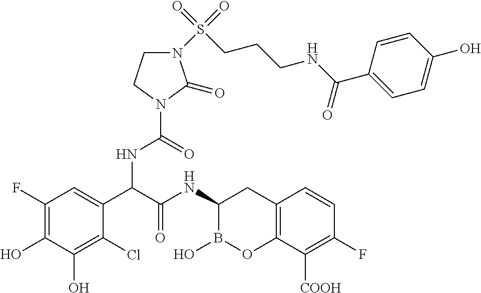 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1ccc(O)cc1] | (3R)-3-(2-(2-chloro-5-fluoro- 3,4-dihydroxyphenyl)-2-(3-((3- (4- hydroxybenzamido)propyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 795.91 | 796 |
| 270 | 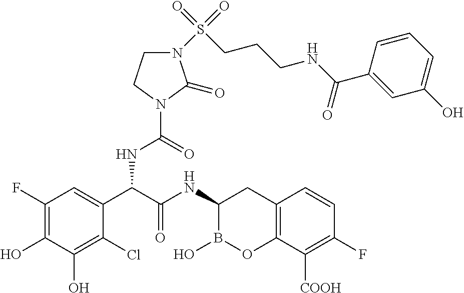 [SMILES: O=C(NCCCS(=O)(=O)N1CCN(C(=O)N[C@H](C(=O)N[C@H]2Cc3ccc(F)c(C(=O)O)c3OB2O)c2cc(F)c(O)c(O)c2Cl)C1=O)c1cccc(O)c1] | (R)-3-((S)-2-(2-chloro-5-fluoro- 3,4-dihydroxyphenyl)-2-(3-((3- (3-hydroxybenzamido)propyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 795.91 | 796 |
| 271 | 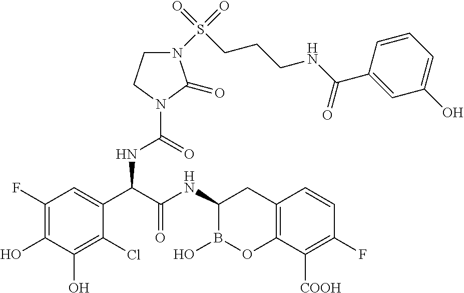 [SMILES: O=C(NCCCS(=O)(=O)N1CCN(C(=O)N[C@@H](C(=O)N[C@H]2Cc3ccc(F)c(C(=O)O)c3OB2O)c2cc(F)c(O)c(O)c2Cl)C1=O)c1cccc(O)c1] | (R)-3-((R)-2-(2-chloro-5-fluoro- 3,4-dihydroxyphenyl)-2-(3-((3- (3-hydroxybenzamido)propyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 795.91 | 796 |
| 272 | 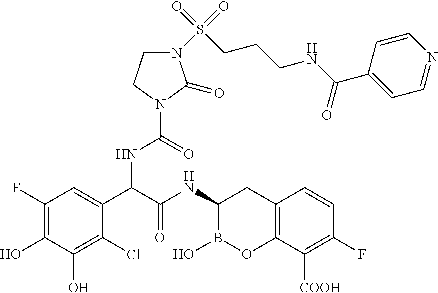 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1ccncc1] | (3R)-3-(2-(2-chloro-5-fluoro- 3,4-dihydroxyphenyl)-2-(3-((3- (isonicotinamido)propyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 780.90 | 781 |
| 273 | 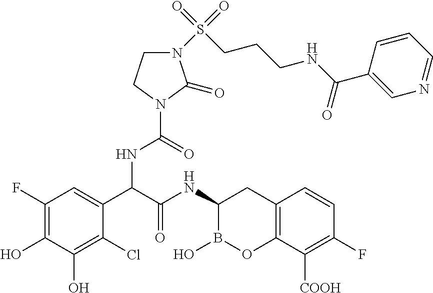 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1cccnc1] | (3R)-3-(2-(2-chloro-5-fluoro- 3,4-dihydroxyphenyl)-2-(3-((3- (nicotinamido)propyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 780.90 | 781 |
| 274 | 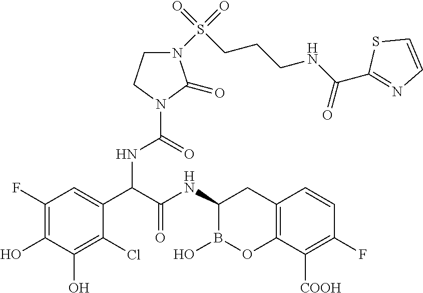 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1nccs1] | (3R)-3-(2-(2-chloro-5-fluoro- 3,4-dihydroxyphenyl)-2-(2-oxo- 3-((3-(thiazole-2- carboxamido)propyl)sulfonyl) imidazolidine-1- carboxamido)acetamido)-7- fluoro-2-hydroxy-3,4-dihydro- 2H-benzo[e][1,2]oxaborinine-8- carboxylic acid | 786.92 | 787 |
| 275 | 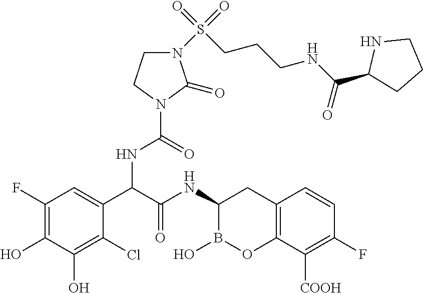 [SMILES: O=C(O)c1c(F)ccc2c1OB(O)[C@@H](NC(=O)C(NC(=O)N1CCN(S(=O)(=O)CCCNC(=O)[C@@H]3CCCN3)C1=O)c1cc(F)c(O)c(O)c1Cl)C2] | (3R)-3-(2-(2-chloro-5-fluoro- 3,4-dihydroxyphenyl)-2-(2-oxo- 3-((3-((S)-pyrrolidine-2- carboxamido)propyl)sulfonyl) imidazolidine-1- carboxamido)acetamido)-7- fluoro-2-hydroxy-3,4-dihydro- 2H-benzo[e][1,2]oxaborinine-8- carboxylic acid | 779.93 | 773 |
| 276 | 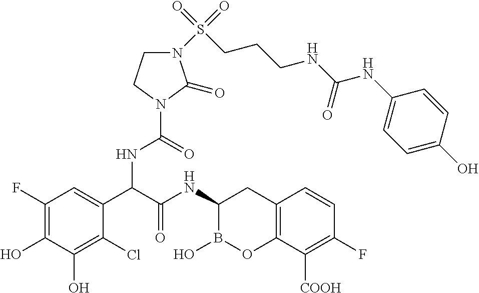 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)Nc1ccc(O)cc1] | (3R)-3-(2-(2-chloro-5-fluoro- 3,4-dihydroxyphenyl)-2-(3-((3- (3-(4- hydroxyphenyl)ureido)propyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 810.93 | 811 |
| 277 | 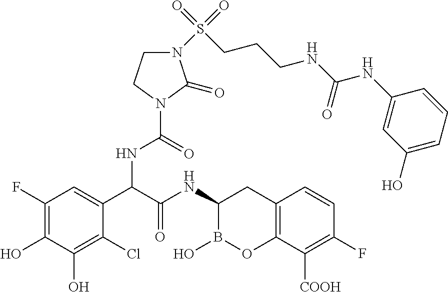 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)Nc1cccc(O)c1] | (3R)-3-(2-(2-chloro-5-fluoro- 3,4-dihydroxyphenyl)-2-(3-((3- (3-(3- hydroxyphenyl)ureido)propyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 810.93 | 811 |
| 278 | 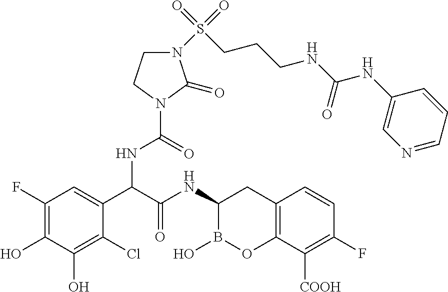 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)Nc1cccnc1] | (3R)-3-(2-(2-chloro-5-fluoro- 3,4-dihydroxyphenyl)-2-(2-oxo- 3-((3-(3-(pyridin-3- yl)ureido)propyl)sulfonyl) imidazolidine-1- carboxamido)acetamido)-7- fluoro-2-hydroxy-3,4-dihydro- 2H-benzo[e][1,2]oxaborinine-8- carboxylic acid | 795.92 | 796 |
| 279 | 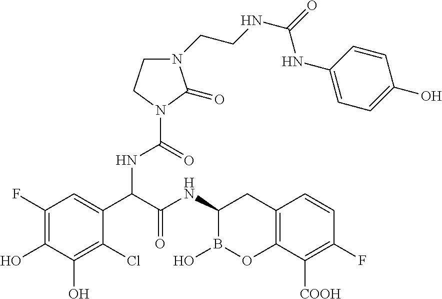 [SMILES: O=C(NCCN1CCN(C(=O)NC(C(=O)N[C@H]2Cc3ccc(F)c(C(=O)O)c3OB2O)c2cc(F)c(O)c(O)c2Cl)C1=O)Nc1ccc(O)cc1] | (R)-3-((R)-2-(2-chloro-5-fluoro- 3,4-dihydroxyphenyl)-2-(3-(2- (3-(4- hydroxyphenyl)ureido)ethyl)-2- oxoimidazolidine-1- carboxamido)acetamido)-7- fluoro-2-hydroxy-3,4-dihydro- 2H-benzo[e][1,2]oxaborinine-8- carboxylic acid | 732.84 | 733 |
| 280 | 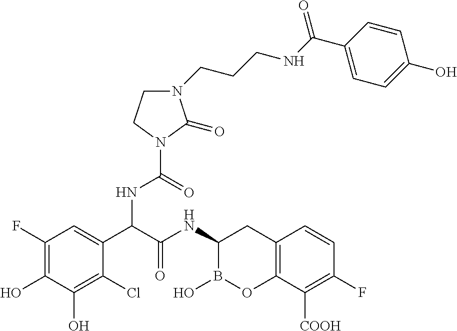 [SMILES: O=C(NCCCN1CCN(C(=O)NC(C(=O)N[C@H]2Cc3ccc(F)c(C(=O)O)c3OB2O)c2cc(F)c(O)c(O)c2Cl)C1=O)c1ccc(O)cc1] | (3R)-3-(2-(2-chloro-5-fluoro- 3,4-dihydroxyphenyl)-2-(3-(3- (4-hydroxybenzamido)propyl)- 2-oxoimidazolidine-1- carboxamido)acetamido)-7- fluoro-2-hydroxy-3,4-dihydro- 2H-benzo[e][1,2]oxaborinine-8- carboxylic acid | 731.85 | 732 |
| 281 | 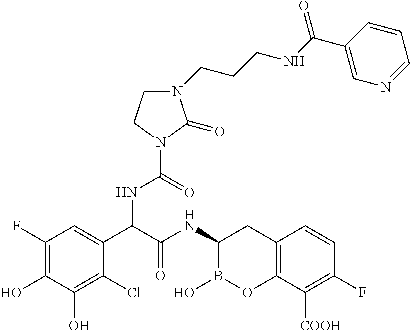 [SMILES: O=C(NCCCN1CCN(C(=O)NC(C(=O)N[C@H]2Cc3ccc(F)c(C(=O)O)c3OB2O)c2cc(F)c(O)c(O)c2Cl)C1=O)c1cccnc1] | (3R)-3-(2-(2-chloro-5-fluoro- 3,4-dihydroxyphenyl)-2-(3-(3- (nicotinamido)propyl)-2- oxoimidazolidine-1- carboxamido)acetamido)-7- fluoro-2-hydroxy-3,4-dihydro- 2H-benzo[e][1,2]oxaborinine-8- carboxylic acid | 716.84 | 717 |
| 282 | 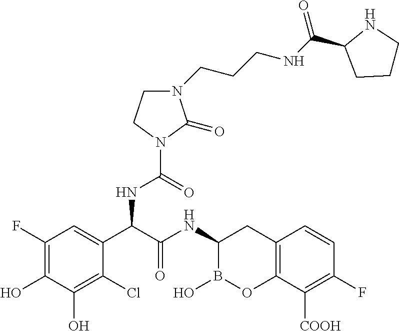 [SMILES: O=C(O)c1c(F)ccc2c1OB(O)[C@@H](NC(=O)[C@H](NC(=O)N1CCN(CCCNC(=O)[C@@H]3CCCN3)C1=O)c1cc(F)c(O)c(O)c1Cl)C2] | (R)-3-((R)-2-(2-chloro-5-fluoro- 3,4-dihydroxyphenyl)-2-(2-oxo- 3-(3-((S)-pyrrolidine-2- carboxamido)propyl) imidazolidine-1- carboxamido)acetamido)-7- fluoro-2-hydroxy-3,4-dihydro- 2H-benzo[e][1,2]oxaborinine-8- carboxylic acid | 708.87 | 709 |
| 283 | 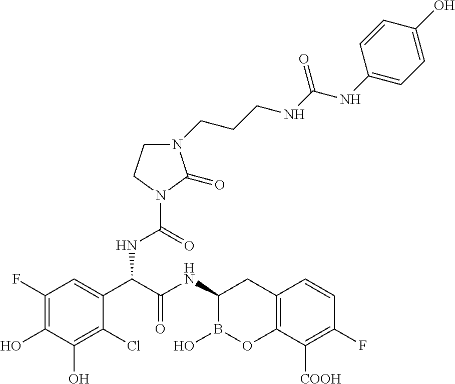 [SMILES: O=C(NCCCN1CCN(C(=O)N[C@H](C(=O)N[C@H]2Cc3ccc(F)c(C(=O)O)c3OB2O)c2cc(F)c(O)c(O)c2Cl)C1=O)Nc1ccc(O)cc1] | (R)-3-((S)-2-(2-chloro-5-fluoro- 3,4-dihydroxyphenyl)-2-(3-(3- (3-(4- hydroxyphenyl)ureido)propyl)- 2-oxoimidazolidine-1- carboxamido)acetamido)-7- fluoro-2-hydroxy-3,4-dihydro- 2H-benzo[e][1,2]oxaborinine-8- carboxylic acid | 746.87 | 747 |
| 284 | 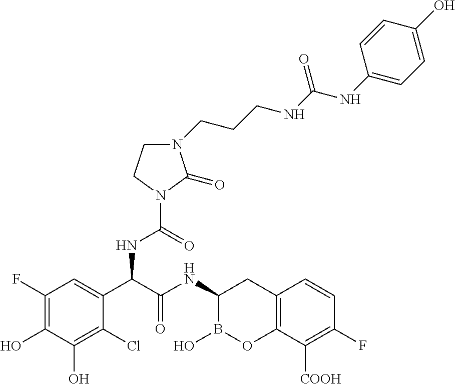 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C1=O)Nc1ccc(O)cc1] | (R)-3-((R)-2-(2-chloro-5-fluoro- 3,4-dihydroxyphenyl)-2-(3-(3- (3-(4- hydroxyphenyl)ureido)propyl)- 2-oxoimidazolidine-1- carboxamido)acetamido)-7- fluoro-2-hydroxy-3,4-dihydro- 2H-benzo[e][1,2]oxaborinine-8- carboxylic acid | 746.87 | 747 |
| 285 | 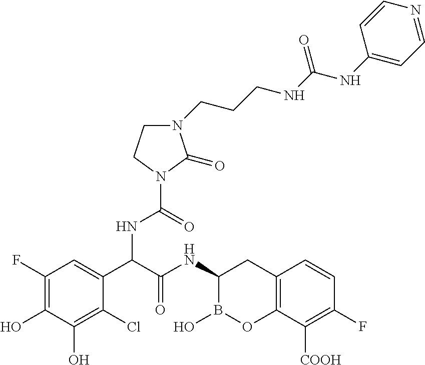 [SMILES: O=C(NCCCN1CCN(C(=O)NC(C(=O)N[C@H]2Cc3ccc(F)c(C(=O)O)c3OB2O)c2cc(F)c(O)c(O)c2Cl)C1=O)Nc1ccncc1] | (3R)-3-(2-(2-chloro-5-fluoro- 3,4-dihydroxyphenyl)-2-(2-oxo- 3-(3-(3-(pyridin-4- yl)ureido)propyl)imidazolidine- 1-carboxamido)acetamido)-7- fluoro-2-hydroxy-3,4-dihydro- 2H-benzo[e][1,2]oxaborinine-8- carboxylic acid | 731.89 | 732 |
| 286 | 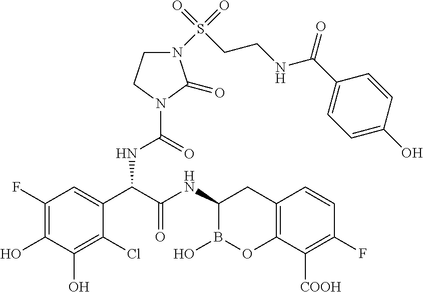 [SMILES: O=C(NCCS(=O)(=O)N1CCN(C(=O)N[C@H](C(=O)N[C@H]2Cc3ccc(F)c(C(=O)O)c3OB2O)c2cc(F)c(O)c(O)c2Cl)C1=O)c1ccc(O)cc1] | (R)-3-((S)-2-(2-chloro-5-fluoro- 3,4-dihydroxyphenyl)-2-(3-((2- (4-hydroxybenzamido)ethyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 781.89 | 782 |
| 287 | 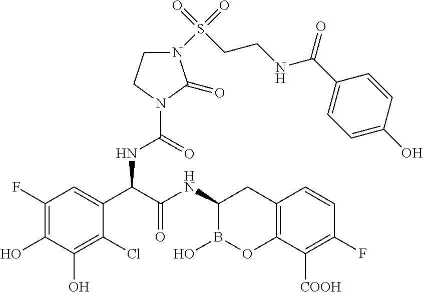 [SMILES: O=C(NCCS(=O)(=O)N1CCN(C(=O)N[C@@H](C(=O)N[C@H]2Cc3ccc(F)c(C(=O)O)c3OB2O)c2cc(F)c(O)c(O)c2Cl)C1=O)c1ccc(O)cc1] | (R)-3-((R)-2-(2-chloro-5-fluoro- 3,4-dihydroxyphenyl)-2-(3-((2- (4-hydroxybenzamido)ethyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 781.89 | 782 |
| 288 | 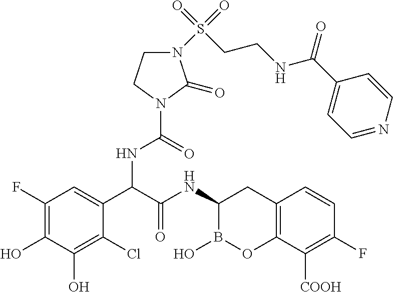 [SMILES: O=C(NCCS(=O)(=O)N1CCN(C(=O)NC(C(=O)N[C@H]2Cc3ccc(F)c(C(=O)O)c3OB2O)c2cc(F)c(O)c(O)c2Cl)C1=O)c1ccncc1] | (3R)-3-(2-(2-chloro-5-fluoro- 3,4-dihydroxyphenyl)-2-(3-((2- (isonicotinamido)ethyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 766.87 | 767 |
| 289 | 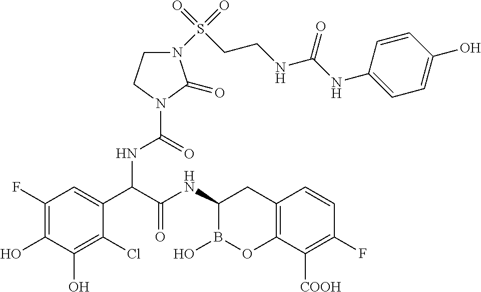 [SMILES: O=C(NCCS(=O)(=O)N1CCN(C(=O)NC(C(=O)N[C@H]2Cc3ccc(F)c(C(=O)O)c3OB2O)c2cc(F)c(O)c(O)c2Cl)C1=O)Nc1ccc(O)cc1] | (3R)-3-(2-(2-chloro-5-fluoro- 3,4-dihydroxyphenyl)-2-(3-((2- (3-(4- hydroxyphenyl)ureido)ethyl) sulfonyl)-2-oxoimidazolidine- 1-carboxamido)acetamido)-7- fluoro-2-hydroxy-3,4-dihydro- 2H-benzo[e][1,2]oxaborinine-8- carboxylic acid | 796.90 | 797 |
| 290 | 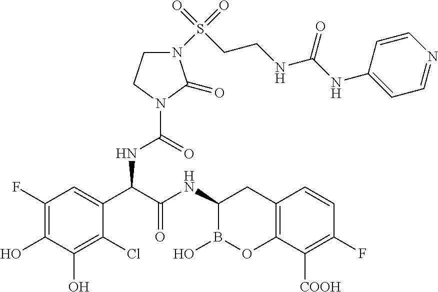 [SMILES: O=C(NCCS(=O)(=O)N1CCN(C(=O)N[C@@H](C(=O)N[C@H]2Cc3ccc(F)c(C(=O)O)c3OB2O)c2cc(F)c(O)c(O)c2Cl)C1=O)Nc1ccncc1] | (R)-3-((R)-2-(2-chloro-5-fluoro- 3,4-dihydroxyphenyl)-2-(2-oxo- 3-((2-(3-(pyridin-4- yl)ureido)ethyl)sulfonyl) imidazolidine-1-carboxamido) acetamido)-7-fluoro-2- hydroxy-3,4-dihydro- 2H-benzo[e][1,2]oxaborinine-8- carboxylic acid | 781.89 | 782 |
| 291 | 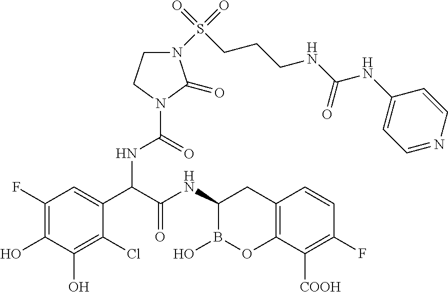 [SMILES: O=C(NCCCS(=O)(=O)N1CCN(C(=O)NC(C(=O)N[C@H]2Cc3ccc(F)c(C(=O)O)c3OB2O)c2cc(F)c(O)c(O)c2Cl)C1=O)Nc1ccncc1] | (3R)-3-(2-(2-chloro-5-fluoro- 3,4-dihydroxyphenyl)-2-(2-oxo- 3-((3-(3-(pyridin-4- yl)ureido)propyl)sulfonyl) imidazolidine-1- carboxamido)acetamido)-7- fluoro-2-hydroxy-3,4-dihydro- 2H-benzo[e][1,2]oxaborinine-8- carboxylic acid | 795.92 | 796 |
| 292 | 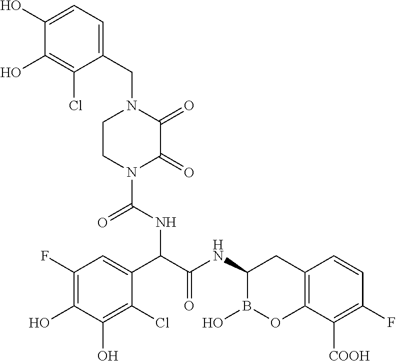 [SMILES: O=C(O)c1c(F)ccc2c1OB(O)[C@@H](NC(=O)C(NC(=O)N1CCN(Cc3ccc(O)c(O)c3Cl)C(=O)C1=O)c1cc(F)c(O)c(O)c1Cl)C2] | (3R)-3-(2-(4-(2-chloro-3,4- dihydroxybenzyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 739.23 | 740 |
| 293 | 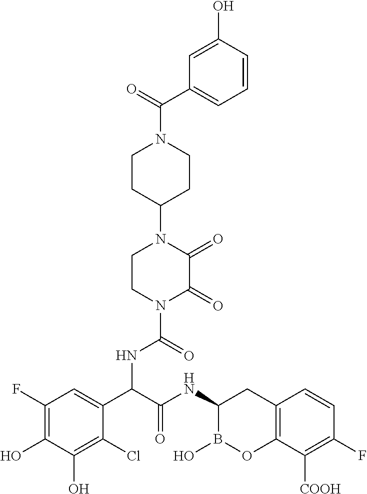 [SMILES: O=C(O)c1c(F)ccc2c1OB(O)[C@@H](NC(=O)C(NC(=O)N1CCN(C3CCN(C(=O)c4cccc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- (3-hydroxybenzoyl)piperidin-4- yl)-2,3-dioxopiperazine-1- carboxamido)acetamido)-7- fluoro-2-hydroxy-3,4-dihydro- 2H-benzo[e][1,2]oxaborinine-8- carboxylic acid | 785.90 | 786 |
| 294 | 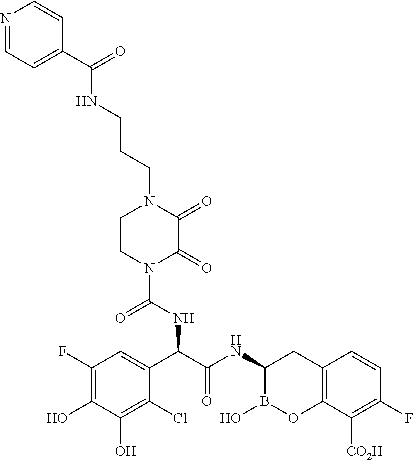 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1ccncc1] | (R)-3-((R)-2-(2-chloro-5-fluoro- 3,4-dihydroxyphenyl)-2-(4-(3- (isonicotinamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 744.85 | 745 |
| 295 | 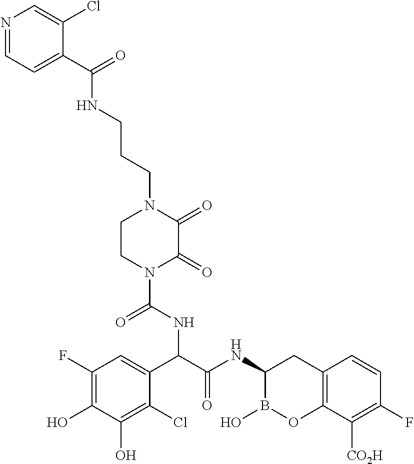 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccncc1Cl] | (3R)-3-(2-(2-chloro-5-fluoro- 3,4-dihydroxyphenyl)-2-(4-(3- (3-chloroisonicotinamido) propyl)-2,3- dioxopiperazine-1- carboxamido)acetamido)-7- fluoro-2-hydroxy-3,4-dihydro- 2H-benzo[e][1,2]oxaborinine-8- carboxylic acid | 779.30 | 780 |
| 296 | 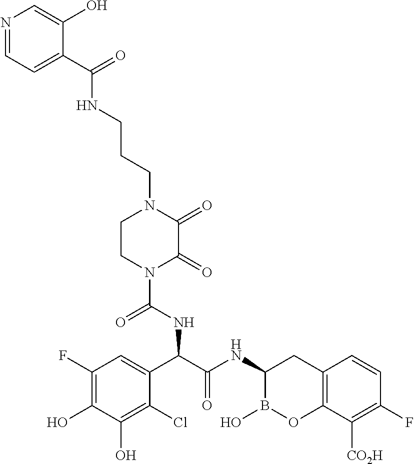 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1ccncc1O] | (R)-3-((R)-2-(2-chloro-5-fluoro- 3,4-dihydroxyphenyl)-2-(4-(3- (3-hydroxyisonicotinamido) propyl)-2,3-dioxopiperazine- 1-carboxamido)acetamido)-7- fluoro-2-hydroxy-3,4-dihydro- 2H-benzo[e][1,2]oxaborinine-8- carboxylic acid | 760.85 | 761 |
| 297 | 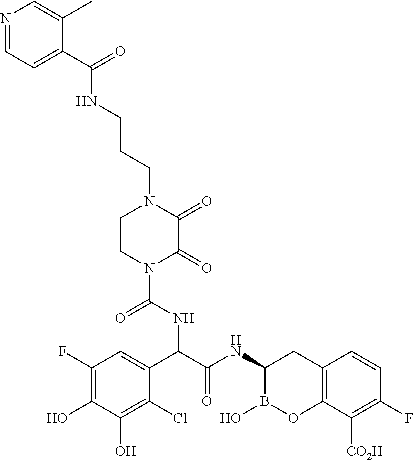 [SMILES: Cc1cnccc1C(=O)NCCCN1CCN(C(=O)NC(C(=O)N[C@H]2Cc3ccc(F)c(C(=O)O)c3OB2O)c2cc(F)c(O)c(O)c2Cl)C(=O)C1=O] | (3R)-3-(2-(2-chloro-5-fluoro- 3,4-dihydroxyphenyl)-2-(4-(3- (3- methylisonicotinamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 758.88 | 759 |
| 298 | 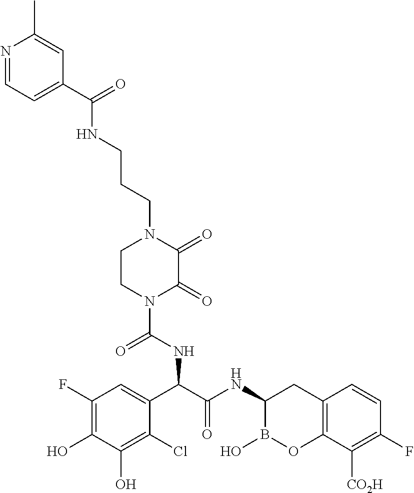 [SMILES: Cc1cc(C(=O)NCCCN2CCN(C(=O)N[C@@H](C(=O)N[C@H]3Cc4ccc(F)c(C(=O)O)c4OB3O)c3cc(F)c(O)c(O)c3Cl)C(=O)C2=O)ccn1] | (R)-3-((R)-2-(2-chloro-5-fluoro- 3,4-dihydroxyphenyl)-2-(4-(3- (2-methylisonicotinamido) propyl)-2,3-dioxopiperazine- 1-carboxamido)acetamido)-7- fluoro-2-hydroxy-3,4-dihydro- 2H-benzo[e][1,2]oxaborinine-8- carboxylic acid | 758.88 | 759 |
| 299 | 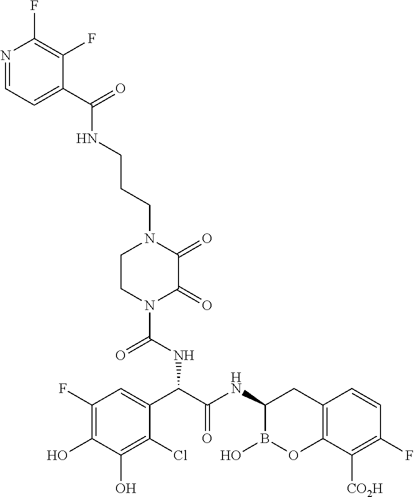 [SMILES: O=C(NCCCN1CCN(C(=O)N[C@H](C(=O)N[C@H]2Cc3ccc(F)c(C(=O)O)c3OB2O)c2cc(F)c(O)c(O)c2Cl)C(=O)C1=O)c1ccnc(F)c1F] | (R)-3-((S)-2-(2-chloro-5-fluoro- 3,4-dihydroxyphenyl)-2-(4-(3- (2,3- difluoroisonicotinamido) propyl)-2,3-dioxopiperazine-1- carboxamido)acetamido)-7- fluoro-2-hydroxy-3,4-dihydro- 2H-benzo[e][1,2]oxaborinine-8- carboxylic acid | 780.83 | 781 |
| 300 | 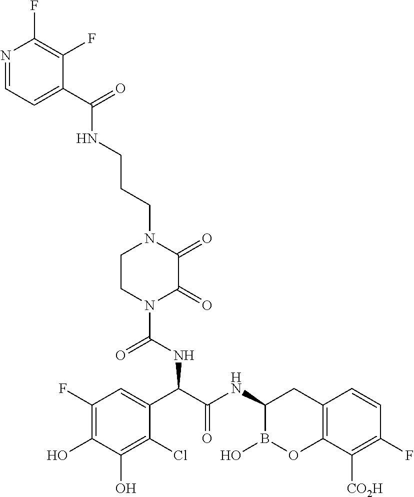 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1ccnc(F)c1F] | (R)-3-((R)-2-(2-chloro-5-fluoro- 3,4-dihydroxyphenyl)-2-(4-(3- (2,3- difluoroisonicotinamido) propyl)-2,3-dioxopiperazine- 1-carboxamido)acetamido)-7- fluoro-2-hydroxy-3,4-dihydro- 2H-benzo[e][1,2]oxaborinine-8- carboxylic acid | 780.83 | 781 |
| 301 | 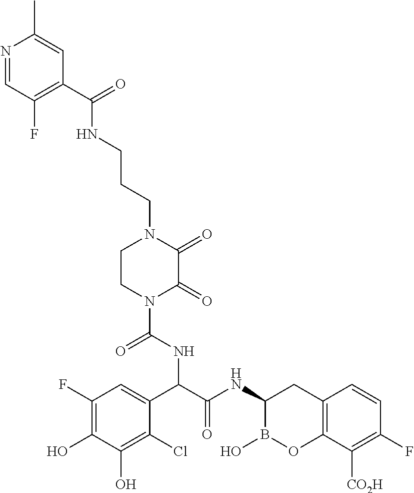 [SMILES: Cc1cc(C(=O)NCCCN2CCN(C(=O)NC(C(=O)N[C@H]3Cc4ccc(F)c(C(=O)O)c4OB3O)c3cc(F)c(O)c(O)c3Cl)C(=O)C2=O)c(F)cn1] | (3R)-3-(2-(2-chloro-5-fluoro- 3,4-dihydroxyphenyl)-2-(4-(3- (5-fluoro-2- methylisonicotinamido) propyl)-2,3-dioxopiperazine- 1-carboxamido)acetamido)-7- fluoro-2-hydroxy-3,4-dihydro- 2H-benzo[e][1,2]oxaborinine-8- carboxylic acid | 776.87 | 777 |
| 302 | 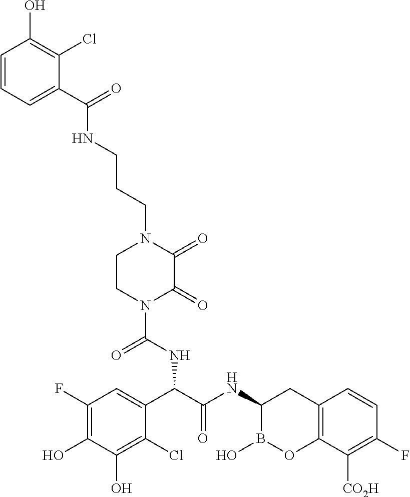 [SMILES: O=C(NCCCN1CCN(C(=O)N[C@H](C(=O)N[C@H]2Cc3ccc(F)c(C(=O)O)c3OB2O)c2cc(F)c(O)c(O)c2Cl)C(=O)C1=O)c1cccc(O)c1Cl] | (R)-3-((S)-2-(4-(3-(2-chloro-3- hydroxybenzamido)propyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 794.31 | 795 |
| 303 | 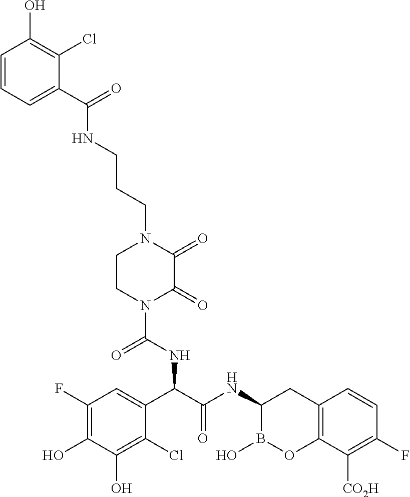 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1cccc(O)c1Cl] | (R)-3-((R)-2-(4-(3-(2-chloro-3- hydroxybenzamido)propyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 794.31 | 795 |
| 304 | 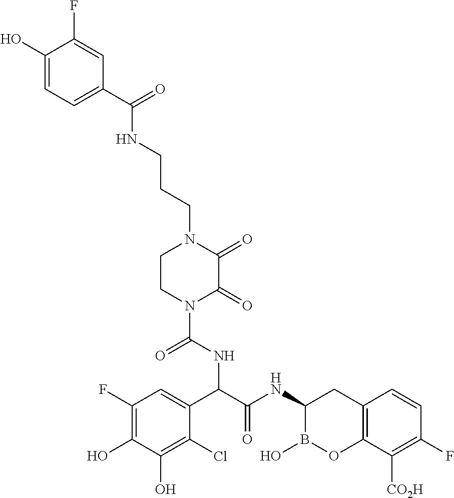 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(O)c(F)c1] | (3R)-3-(2-(2-chloro-5-fluoro- 3,4-dihydroxyphenyl)-2-(4-(3- (3-fluoro-4- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 777.85 | 778 |
| 305 | 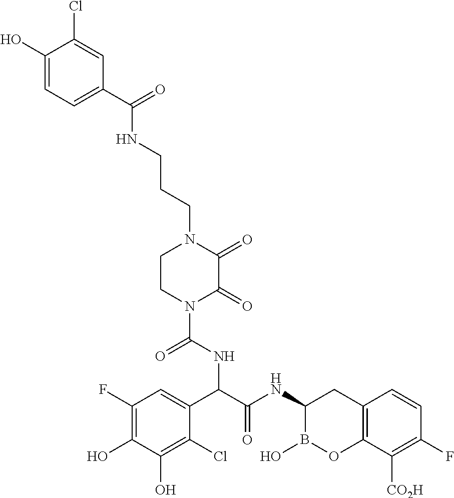 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(O)c(Cl)c1] | (3R)-3-(2-(4-(3-(3-chloro-4- hydroxybenzamido)propyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 794.31 | 795 |
| 306 | 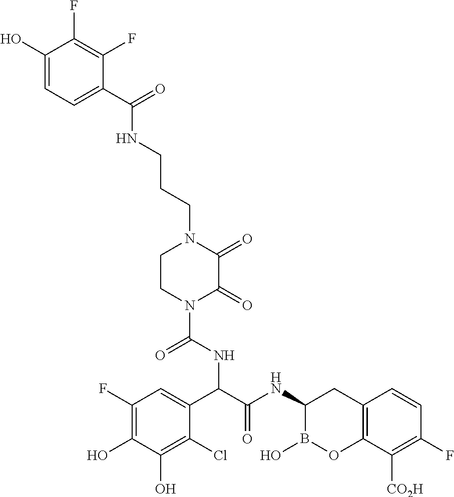 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(O)c(F)c1F] | (3R)-3-(2-(2-chloro-5-fluoro- 3,4-dihydroxyphenyl)-2-(4-(3- (2,3-difluoro-4- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 795.85 | 796 |
| 307 | 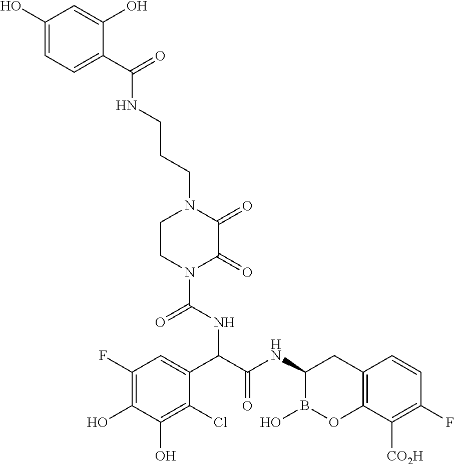 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(O)cc1O] | (3R)-3-(2-(2-chloro-5-fluoro- 3,4-dihydroxyphenyl)-2-(4-(3- (2,4- dihydroxybenzamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 775.86 | 776 |
| 308 | 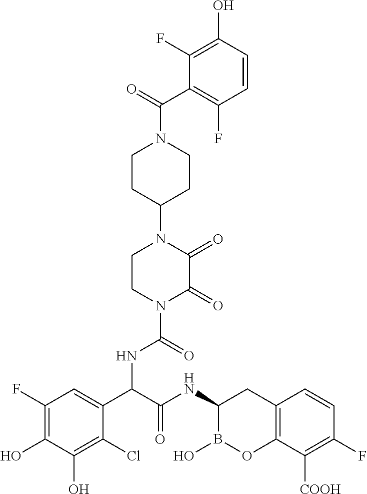 [SMILES: O=C(O)c1c(F)ccc2c1OB(O)[C@@H](NC(=O)C(NC(=O)N1CCN(C3CCN(C(=O)c4c(F)ccc(O)c4F)CC3)C(=O)C1=O)c1cc(F)c(O)c(O)c1Cl)C2] | (3R)-3-(2-(2-chloro-5-fluoro- 3,4-dihydroxyphenyl)-2-(4-(1- (2,6-difluoro-3- hydroxybenzoyl)piperidin-4- yl)-2,3-dioxopiperazine-1- carboxamido)acetamido)-7- fluoro-2-hydroxy-3,4-dihydro- 2H-benzo[e][1,2]oxaborinine-8- carboxylic acid | 821.88 | 822 |
| 309 | 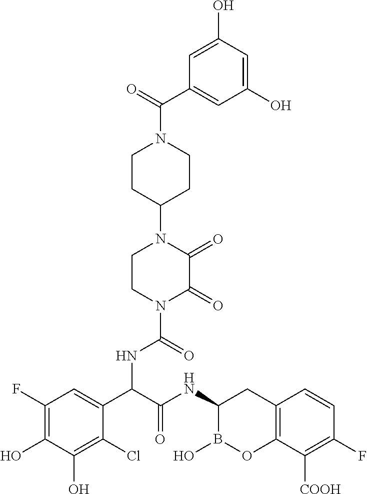 [SMILES: O=C(O)c1c(F)ccc2c1OB(O)[C@@H](NC(=O)C(NC(=O)N1CCN(C3CCN(C(=O)c4cc(O)cc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- (3,5- dihydroxybenzoyl)piperidin-4- yl)-2,3-dioxopiperazine-1- carboxamido)acetamido)-7- fluoro-2-hydroxy-3,4-dihydro- 2H-benzo[e][1,2]oxaborinine-8- carboxylic acid | 801.90 | 802 |
| 310 | 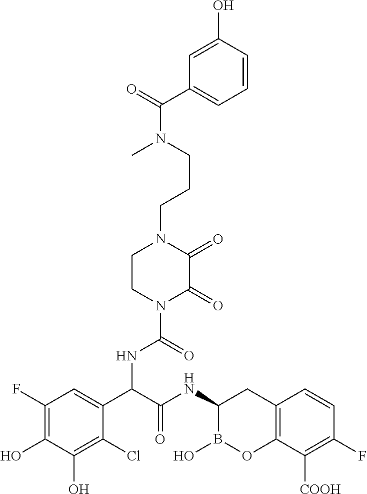 [SMILES: CN(CCCN1CCN(C(=O)NC(C(=O)N[C@H]2Cc3ccc(F)c(C(=O)O)c3OB2O)c2cc(F)c(O)c(O)c2Cl)C(=O)C1=O)C(=O)c1cccc(O)c1] | (3R)-3-(2-(2-chloro-5-fluoro- 3,4-dihydroxyphenyl)-2-(4-(3- (3-hydroxy-N- methylbenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 773.89 | 774 |
| 400 | 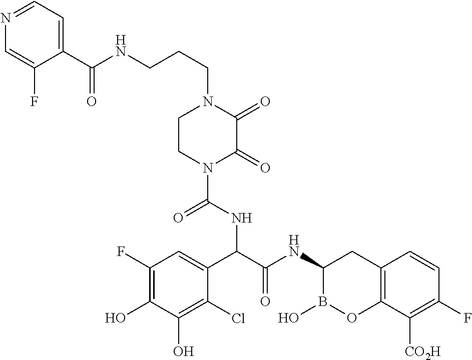 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccncc1F] | (3R)-3-(2-(2-chloro-5-fluoro- 3,4-dihydroxyphenyl)-2-(4-(3- (3- fluoroisonicotinamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 762.84 | 763 |
| 401 | 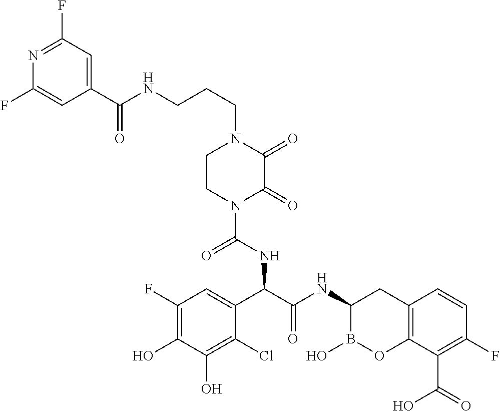 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1cc(F)nc(F)c1] | (R)-3-((R)-2-(2-chloro-5-fluoro- 3,4-dihydroxyphenyl)-2-(4-(3- (2,6- difluoroisonicotinamido) propyl)-2,3-dioxopiperazine- 1-carboxamido)acetamido)-7- fluoro-2-hydroxy-3,4- dihydro- 2H-benzo[e][1,2]oxaborinine-8- carboxylic acid | 780.83 | 781 |
| 402 | 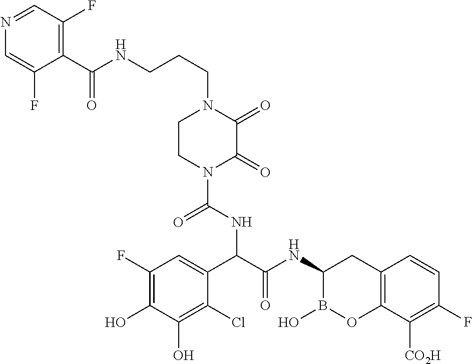 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1c(F)cncc1F] | (3R)-3-(2-(2-chloro-5-fluoro- 3,4-dihydroxyphenyl)-2-(4-(3- (3,5- difluoroisonicotinamido) propyl)-2,3-dioxopiperazine-1- carboxamido)acetamido)-7- fluoro-2-hydroxy-3,4-dihydro- 2H-benzo[e][1,2]oxaborinine-8- carboxylic acid | 780.83 | 781 |
| 403 | 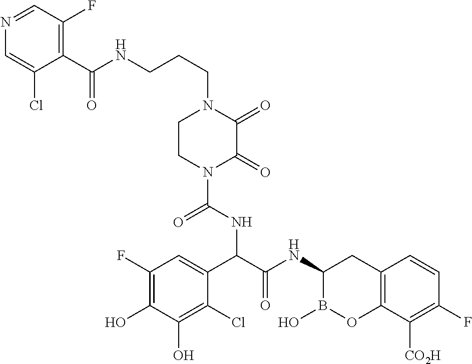 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1c(F)cncc1Cl] | (3R)-3-(2-(2-chloro-5-fluoro- 3,4-dihydroxyphenyl)-2-(4-(3- (3-chloro-5- fluoroisonicotinamido) propyl)-2,3-dioxopiperazine-1- carboxamido)acetamido)-7- fluoro-2-hydroxy-3,4-dihydro- 2H-benzo[e][1,2]oxaborinine-8- carboxylic acid | 797.29 | 797 |
| 404 | 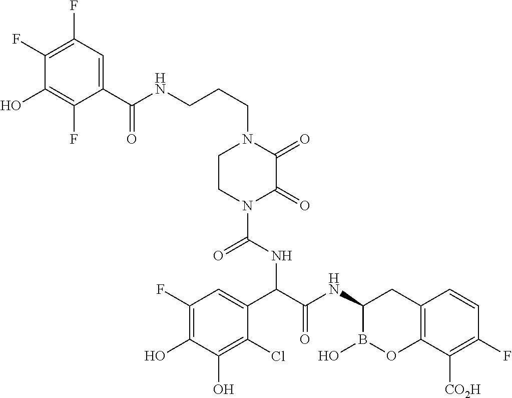 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cc(F)c(F)c(O)c1F] | (3R)-3-(2-(2-chloro-5-fluoro- 3,4-dihydroxyphenyl)-2-(2,3- dioxo-4-(3-(2,4,5-trifluoro-3- hydroxybenzamido)propyl) piperazine-1- carboxamido)acetamido)-7- fluoro-2-hydroxy-3,4-dihydro- 2H-benzo[e][1,2]oxaborinine-8- carboxylic acid | 813.84 | 814 |
| 405 | 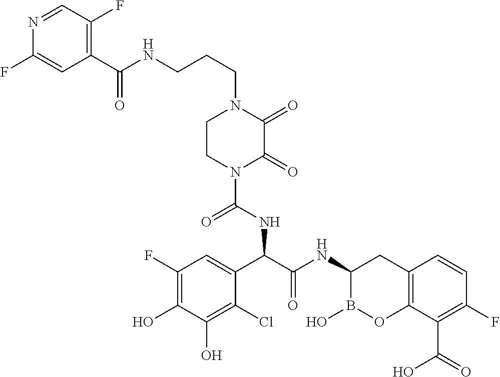 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1cc(F)ncc1F] | (R)-3-((R)-2-(2-chloro-5-fluoro- 3,4-dihydroxyphenyl)-2-(4-(3- (2,5- difluoroisonicotinamido) propyl)-2,3-dioxopiperazine-1- carboxamido)acetamido)-7- fluoro-2-hydroxy-3,4-dihydro- 2H-benzo[e][1,2]oxaborinine-8- carboxylic acid | 780.83 | 781 |
| 406 | 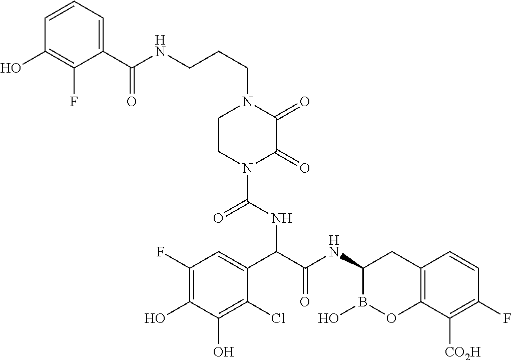 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cccc(O)c1F] | (3R)-3-(2-(2-chloro-5-fluoro- 3,4-dihydroxyphenyl)-2-(4-(3- (2-fluoro-3- hydroxybenzamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 777.85 | 778 |
| 407 | 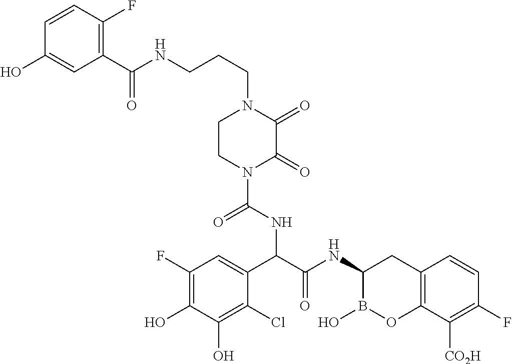 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cc(O)ccc1F] | (3R)-3-(2-(2-chloro-5-fluoro- 3,4-dihydroxyphenyl)-2-(4-(3- (2-fluoro-5- hydroxybenzamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 777.85 | 778 |
| 408 | 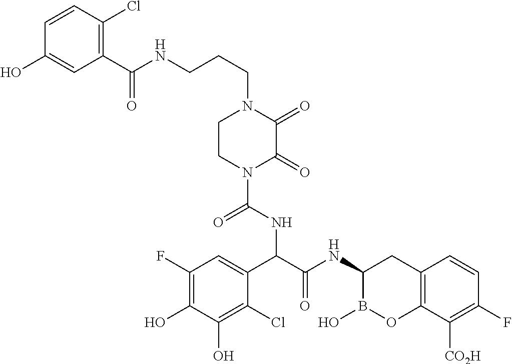 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cc(O)ccc1Cl] | (3R)-3-(2-(2-chloro-5-fluoro- 3,4-dihydroxyphenyl)-2-(4-(3- (2-chloro-5- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 794.31 | 794 |
| 409 | 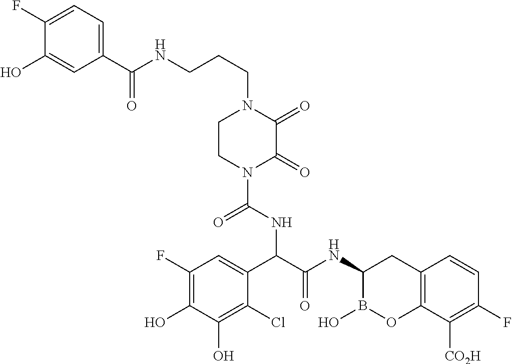 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(F)c(O)c1] | (3R)-3-(2-(2-chloro-5-fluoro- 3,4-dihydroxyphenyl)-2-(4-(3- (4-fluoro-3- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 777.85 | 778 |
| 410 | 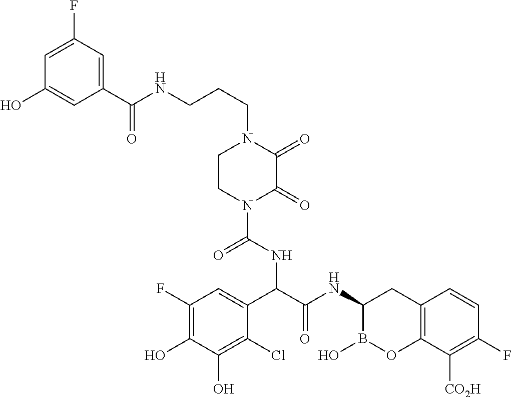 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cc(O)cc(F)c1] | (3R)-3-(2-(2-chloro-5-fluoro- 3,4-dihydroxyphenyl)-2-(4-(3- (3-fluoro-5- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 777.85 | 778 |
| 411 | 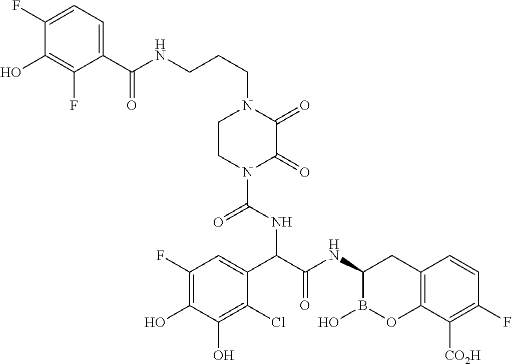 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(F)c(O)c1F] | (3R)-3-(2-(2-chloro-5-fluoro- 3,4-dihydroxyphenyl)-2-(4-(3- (2,4-difluoro-3- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 795.84 | 796 |
| 412 | 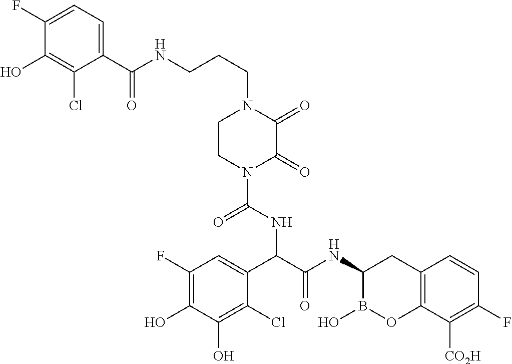 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(F)c(O)c1Cl] | (3R)-3-(2-(4-(3-(2-chloro-4- fluoro-3- hydroxybenzamido)propyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 812.30 | 812 |
| 413 | 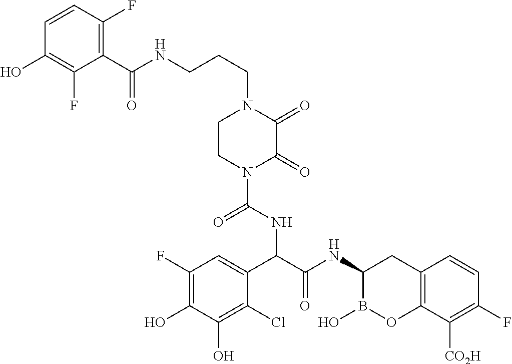 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1c(F)ccc(O)c1F] | (3R)-3-(2-(2-chloro-5-fluoro- 3,4-dihydroxyphenyl)-2-(4-(3- (2,6-difluoro-3- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 795.84 | 796 |
| 414 | 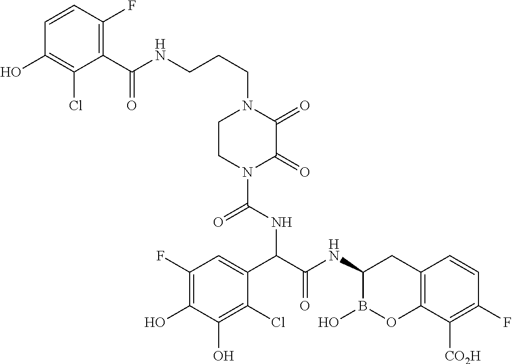 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1c(F)ccc(O)c1Cl] | (3R)-3-(2-(2-chloro-5-fluoro- 3,4-dihydroxyphenyl)-2-(4-(3- (2-chloro-6-fluoro-3- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 812.30 | 812 |
| 415 | 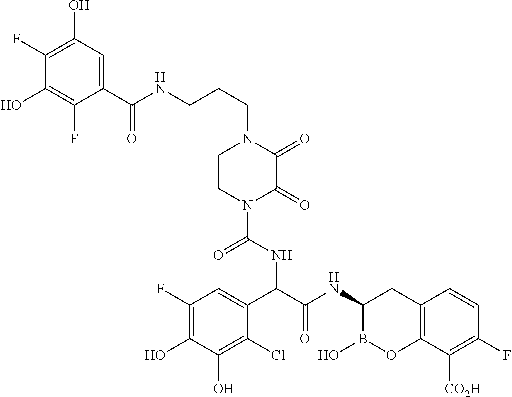 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cc(O)c(F)c(O)c1F] | (3R)-3-(2-(2-chloro-5-fluoro- 3,4-dihydroxyphenyl)-2-(4-(3- (2,4-difluoro-3,5- dihydroxybenzamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 811.84 | 812 |
| 416 | 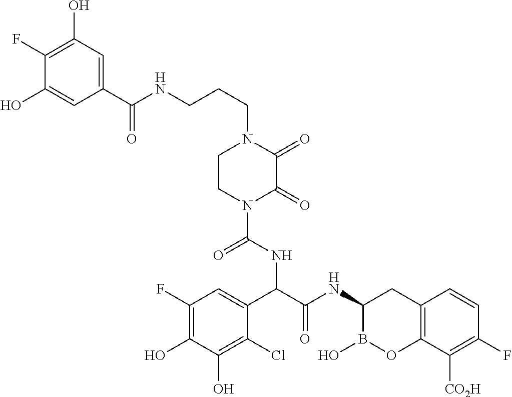 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cc(O)c(F)c(O)c1] | (3R)-3-(2-(2-chloro-5-fluoro- 3,4-dihydroxyphenyl)-2-(4-(3- (4-fluoro-3,5- dihydroxybenzamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 793.85 | 794 |
| 417 | 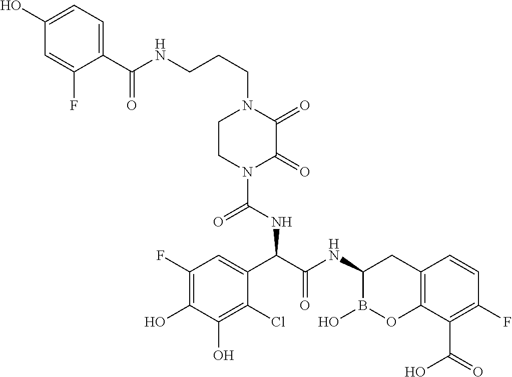 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1ccc(O)cc1F] | (R)-3-((R)-2-(2-chloro-5-fluoro- 3,4-dihydroxyphenyl)-2-(4-(3- (2-fluoro-4- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 777.85 | 778 |
| 418 | 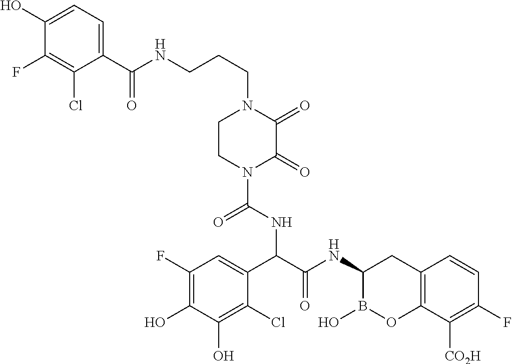 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(O)c(F)c1Cl] | (3R)-3-(2-(4-(3-(2-chloro-3- fluoro-4- hydroxybenzamido)propyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 812.30 | 812 |
| 419 | 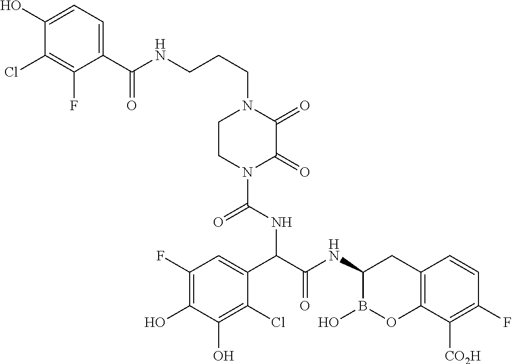 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccc(O)c(Cl)c1F] | (3R)-3-(2-(4-(3-(3-chloro-2- fluoro-4- hydroxybenzamido)propyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 812.30 | 812 |
| 420 | 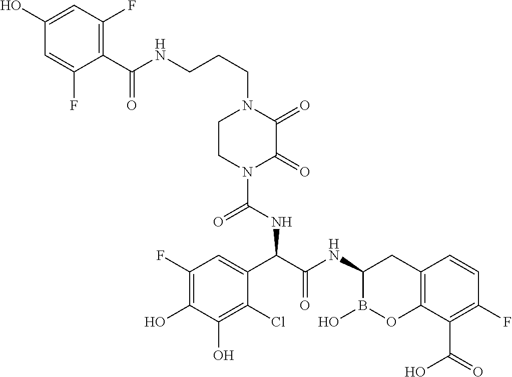 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1c(F)cc(O)cc1F] | (R)-3-((R)-2-(2-chloro-5-fluoro- 3,4-dihydroxyphenyl)-2-(4-(3- (2,6-difluoro-4- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 795.84 | 796 |
| 421 | 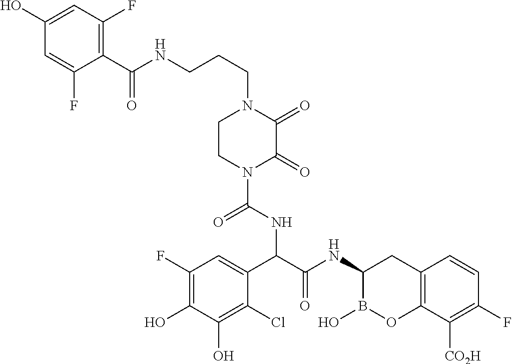 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1c(F)cc(O)cc1F] | (3R)-3-(2-(2-chloro-5-fluoro- 3,4-dihydroxyphenyl)-2-(4-(3- (2-chloro-6-fluoro-4- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 812.30 | 812 |
| 422 | 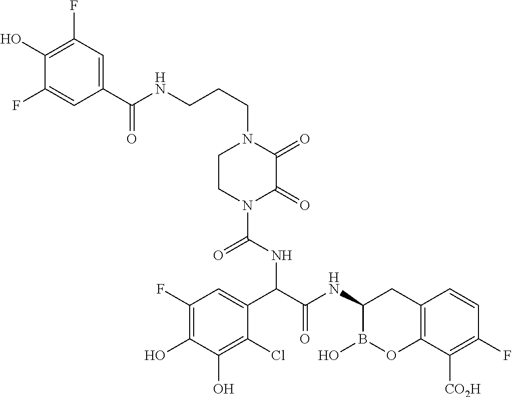 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cc(F)c(O)c(F)c1] | (3R)-3-(2-(2-chloro-5-fluoro- 3,4-dihydroxyphenyl)-2-(4-(3- (3,5-difluoro-4- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 795.84 | 796 |
| 423 | 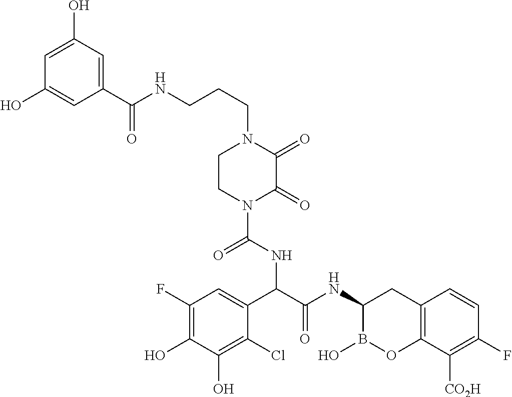 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1cc(O)cc(O)c1] | (3R)-3-(2-(2-chloro-5-fluoro- 3,4-dihydroxyphenyl)-2-(4-(3- (3,5- dihydroxybenzamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 775.86 | 776 |
| 424 | 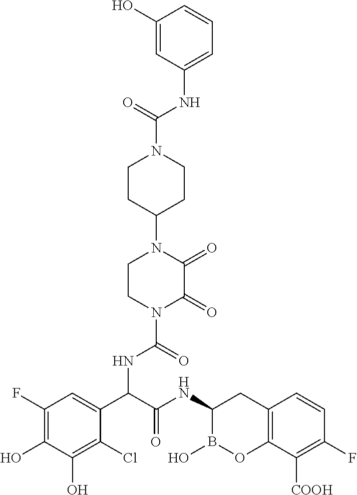 [SMILES: O=C(O)c1c(F)ccc2c1OB(O)[C@@H](NC(=O)C(NC(=O)N1CCN(C3CCN(C(=O)Nc4cccc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((3- hydroxyphenyl)carbamoyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 800.92 | 801/803 |
| 425 | 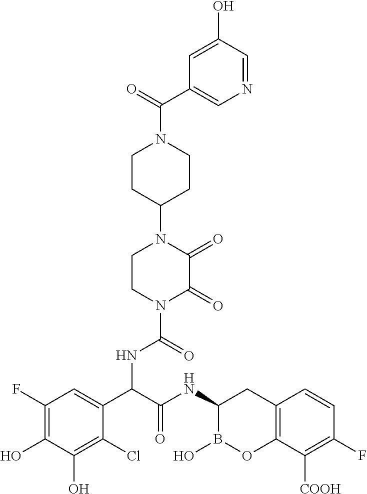 [SMILES: O=C(O)c1c(F)ccc2c1OB(O)[C@@H](NC(=O)C(NC(=O)N1CCN(C3CCN(C(=O)c4cncc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- (5-hydroxynicotinoyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 786.89 | 787/789 |
| 426 | 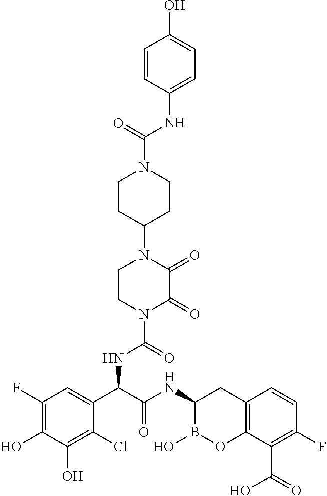 [SMILES: O=C(O)c1c(F)ccc2c1OB(O)[C@@H](NC(=O)[C@H](NC(=O)N1CCN(C3CCN(C(=O)Nc4ccc(O)cc4)CC3)C(=O)C1=O)c1cc(F)c(O)c(O)c1Cl)C2] | (R)-3-((R)-2-(2-chloro-5-fluoro- 3,4-dihydroxyphenyl)-2-(4-(1- ((4- hydroxyphenyl)carbamoyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 800.92 | 801 |
| 427 | 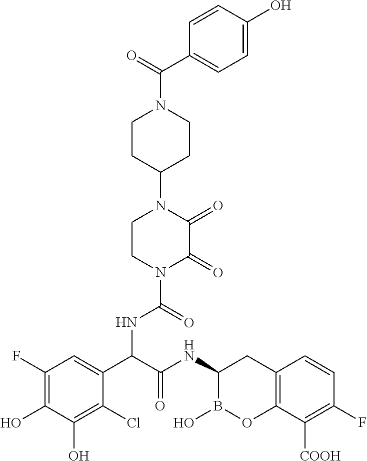 [SMILES: O=C(O)c1c(F)ccc2c1OB(O)[C@@H](NC(=O)C(NC(=O)N1CCN(C3CCN(C(=O)c4ccc(O)cc4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- (4-hydroxybenzoyl)piperidin-4- yl)-2,3-dioxopiperazine-1- carboxamido)acetamido)-7- fluoro-2-hydroxy-3,4-dihydro- 2H-benzo[e][1,2]oxaborinine-8- carboxylic acid | 785.90 | 786/788 |
| 428 | 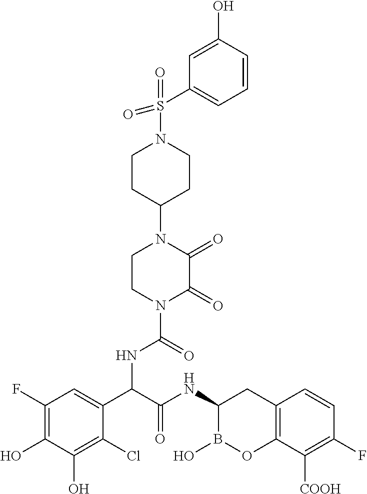 [SMILES: O=C(O)c1c(F)ccc2c1OB(O)[C@@H](NC(=O)C(NC(=O)N1CCN(C3CCN(S(=O)(=O)c4cccc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((3- hydroxyphenyl)sulfonyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 821.95 | 822/824 |
| 429 | 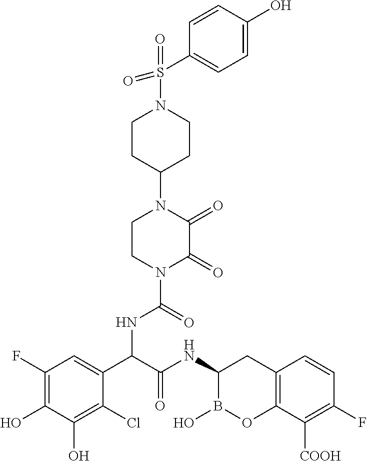 [SMILES: O=C(O)c1c(F)ccc2c1OB(O)[C@@H](NC(=O)C(NC(=O)N1CCN(C3CCN(S(=O)(=O)c4ccc(O)cc4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((4- hydroxyphenyl)sulfonyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 821.95 | 822/824 |
| 430 | 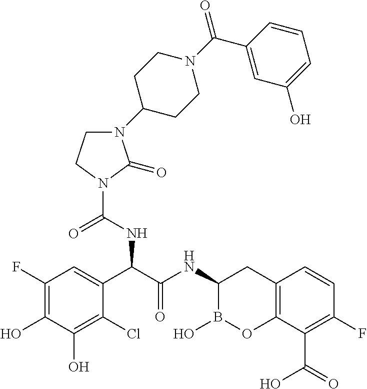 [SMILES: O=C(O)c1c(F)ccc2c1OB(O)[C@@H](NC(=O)[C@H](NC(=O)N1CCN(C3CCN(C(=O)c4cccc(O)c4)CC3)C1=O)c1cc(F)c(O)c(O)c1Cl)C2] | (R)-3-((R)-2-(2-chloro-5-fluoro- 3,4-dihydroxyphenyl)-2-(3-(1- (3-hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 757.89 | 758 |
| 431 | 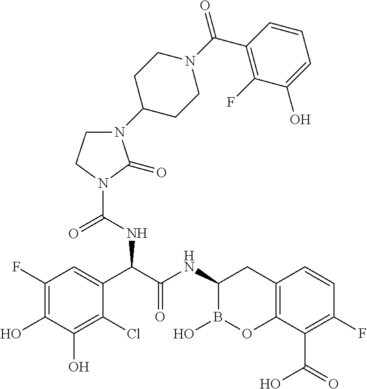 [SMILES: O=C(O)c1c(F)ccc2c1OB(O)[C@@H](NC(=O)[C@H](NC(=O)N1CCN(C3CCN(C(=O)c4cccc(O)c4F)CC3)C1=O)c1cc(F)c(O)c(O)c1Cl)C2] | (R)-3-((R)-2-(2-chloro-5-fluoro- 3,4-dihydroxyphenyl)-2-(3-(1- (2-fluoro-3- hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 775.88 | 776 |
| 432 | 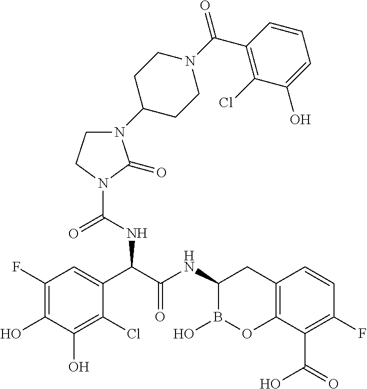 [SMILES: O=C(O)c1c(F)ccc2c1OB(O)[C@@H](NC(=O)[C@H](NC(=O)N1CCN(C3CCN(C(=O)c4cccc(O)c4Cl)CC3)C1=O)c1cc(F)c(O)c(O)c1Cl)C2] | (R)-3-((R)-2-(3-(1-(2-chloro-3- hydroxybenzoyl)piperidin-4- yl)-2-oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 792.33 | 792 |
| 433 | 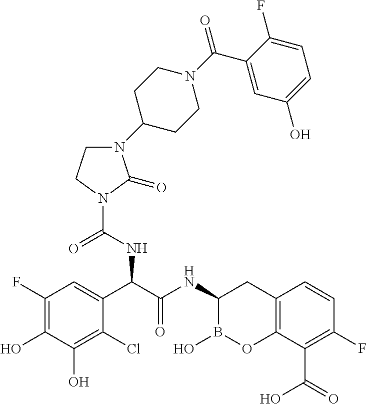 [SMILES: O=C(O)c1c(F)ccc2c1OB(O)[C@@H](NC(=O)[C@H](NC(=O)N1CCN(C3CCN(C(=O)c4cc(O)ccc4F)CC3)C1=O)c1cc(F)c(O)c(O)c1Cl)C2] | (R)-3-((R)-2-(2-chloro-5-fluoro- 3,4-dihydroxyphenyl)-2-(3-(1- (2-fluoro-5- hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 775.88 | 776 |
| 434 | 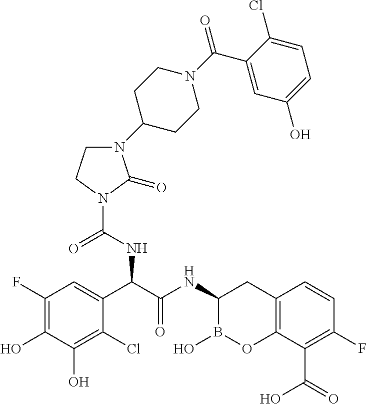 [SMILES: O=C(O)c1c(F)ccc2c1OB(O)[C@@H](NC(=O)[C@H](NC(=O)N1CCN(C3CCN(C(=O)c4cc(O)ccc4Cl)CC3)C1=O)c1cc(F)c(O)c(O)c1Cl)C2] | (R)-3-((R)-2-(2-chloro-5-fluoro- 3,4-dihydroxyphenyl)-2-(3-(1- (2-chloro-5- hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 792.33 | 792 |
| 435 | 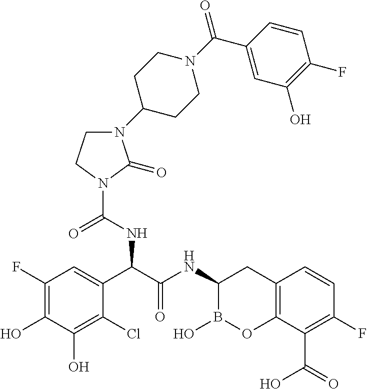 [SMILES: O=C(O)c1c(F)ccc2c1OB(O)[C@@H](NC(=O)[C@H](NC(=O)N1CCN(C3CCN(C(=O)c4ccc(F)c(O)c4)CC3)C1=O)c1cc(F)c(O)c(O)c1Cl)C2] | (R)-3-((R)-2-(2-chloro-5-fluoro- 3,4-dihydroxyphenyl)-2-(3-(1- (4-fluoro-3- hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 775.88 | 776 |
| 436 | 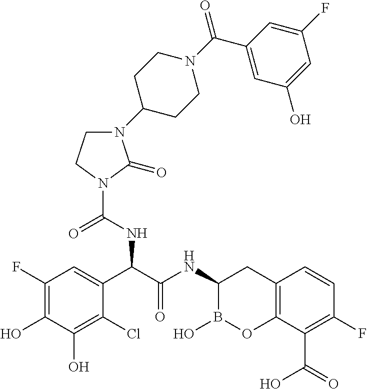 [SMILES: O=C(O)c1c(F)ccc2c1OB(O)[C@@H](NC(=O)[C@H](NC(=O)N1CCN(C3CCN(C(=O)c4cc(O)cc(F)c4)CC3)C1=O)c1cc(F)c(O)c(O)c1Cl)C2] | (R)-3-((R)-2-(2-chloro-5-fluoro- 3,4-dihydroxyphenyl)-2-(3-(1- (3-fluoro-5- hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 775.88 | 776 |
| 437 | 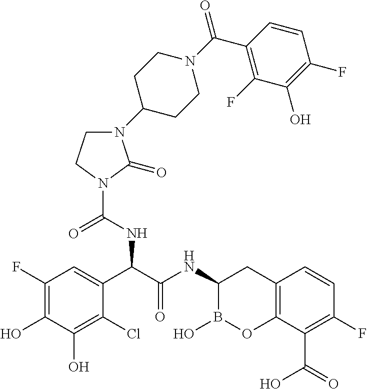 [SMILES: O=C(O)c1c(F)ccc2c1OB(O)[C@@H](NC(=O)[C@H](NC(=O)N1CCN(C3CCN(C(=O)c4ccc(F)c(O)c4F)CC3)C1=O)c1cc(F)c(O)c(O)c1Cl)C2] | (R)-3-((R)-2-(2-chloro-5-fluoro- 3,4-dihydroxyphenyl)-2-(3-(1- (2,4-difluoro-3- hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 793.87 | 794 |
| 438 | 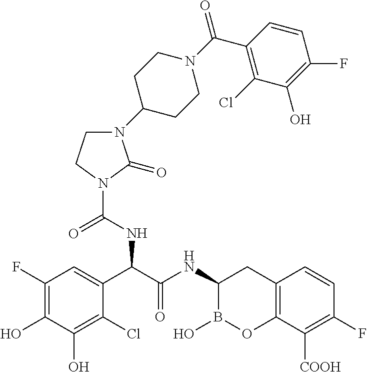 [SMILES: O=C(O)c1c(F)ccc2c1OB(O)[C@@H](NC(=O)[C@H](NC(=O)N1CCN(C3CCN(C(=O)c4ccc(F)c(O)c4Cl)CC3)C1=O)c1cc(F)c(O)c(O)c1Cl)C2] | (R)-3-((R)-2-(3-(1-(2-chloro-4- fluoro-3- hydroxybenzoyl)piperidin-4- yl)-2-oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 810.32 | 810 |
| 439 | 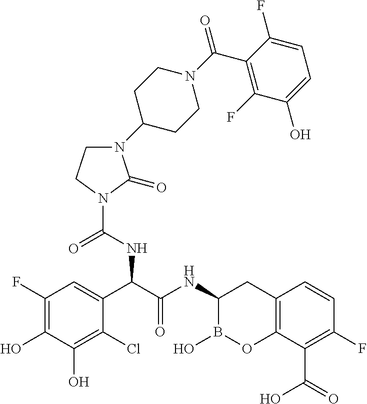 [SMILES: O=C(O)c1c(F)ccc2c1OB(O)[C@@H](NC(=O)[C@H](NC(=O)N1CCN(C3CCN(C(=O)c4c(F)ccc(O)c4F)CC3)C1=O)c1cc(F)c(O)c(O)c1Cl)C2] | (R)-3-((R)-2-(2-chloro-5-fluoro- 3,4-dihydroxyphenyl)-2-(3-(1- (2,6-difluoro-3- hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 793.87 | 794 |
| 440 | 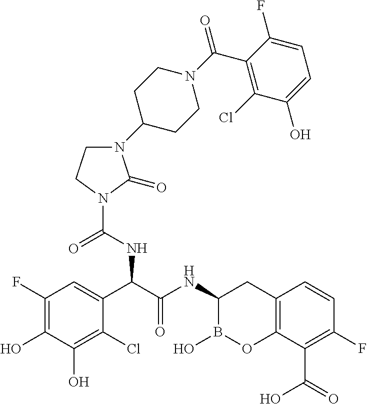 [SMILES: O=C(O)c1c(F)ccc2c1OB(O)[C@@H](NC(=O)[C@H](NC(=O)N1CCN(C3CCN(C(=O)c4c(F)ccc(O)c4Cl)CC3)C1=O)c1cc(F)c(O)c(O)c1Cl)C2] | (R)-3-((R)-2-(2-chloro-5-fluoro- 3,4-dihydroxyphenyl)-2-(3-(1- (2-chloro-6-fluoro-3- hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 812.34 | 810 |
| 441 | 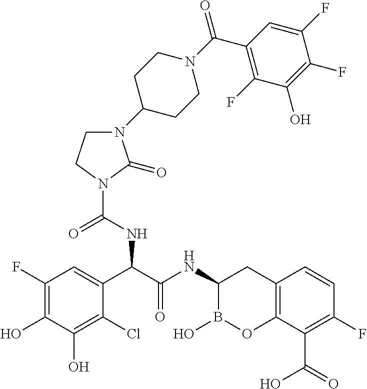 [SMILES: O=C(O)c1c(F)ccc2c1OB(O)[C@@H](NC(=O)[C@H](NC(=O)N1CCN(C3CCN(C(=O)c4cc(F)c(F)c(O)c4F)CC3)C1=O)c1cc(F)c(O)c(O)c1Cl)C2] | (R)-3-((R)-2-(2-chloro-5-fluoro- 3,4-dihydroxyphenyl)-2-(2-oxo- 3-(1-(2,4,5-trifluoro-3- hydroxybenzoyl)piperidin-4- yl)imidazolidine-1- carboxamido)acetamido)-7- fluoro-2-hydroxy-3,4-dihydro- 2H-benzo[e][1,2]oxaborinine-8- carboxylic acid | 811.86 | 812 |
| 442 | 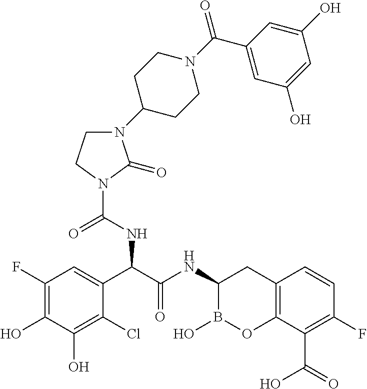 [SMILES: O=C(O)c1c(F)ccc2c1OB(O)[C@@H](NC(=O)[C@H](NC(=O)N1CCN(C3CCN(C(=O)c4cc(O)cc(O)c4)CC3)C1=O)c1cc(F)c(O)c(O)c1Cl)C2] | (R)-3-((R)-2-(2-chloro-5-fluoro- 3,4-dihydroxyphenyl)-2-(3-(1- (3,5- dihydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 773.89 | 774 |
| 443 | 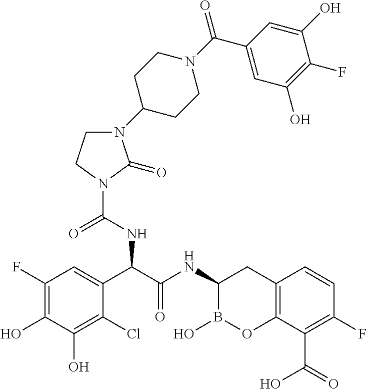 [SMILES: O=C(O)c1c(F)ccc2c1OB(O)[C@@H](NC(=O)[C@H](NC(=O)N1CCN(C3CCN(C(=O)c4cc(O)c(F)c(O)c4)CC3)C1=O)c1cc(F)c(O)c(O)c1Cl)C2] | (R)-3-((R)-2-(2-chloro-5-fluoro- 3,4-dihydroxyphenyl)-2-(3-(1- (4-fluoro-3,5- dihydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 791.88 | 792 |
| 444 | 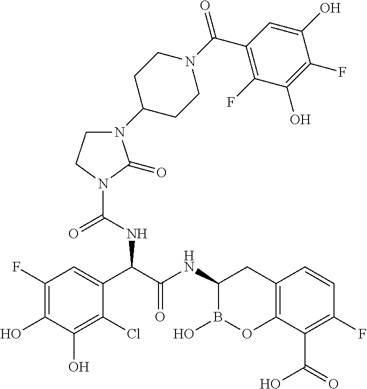 [SMILES: O=C(O)c1c(F)ccc2c1OB(O)[C@@H](NC(=O)[C@H](NC(=O)N1CCN(C3CCN(C(=O)c4cc(O)c(F)c(O)c4F)CC3)C1=O)c1cc(F)c(O)c(O)c1Cl)C2] | (R)-3-((R)-2-(2-chloro-5-fluoro- 3,4-dihydroxyphenyl)-2-(3-(1- (2,4-difluoro-3,5- dihydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 809.87 | 810 |
| 445 | 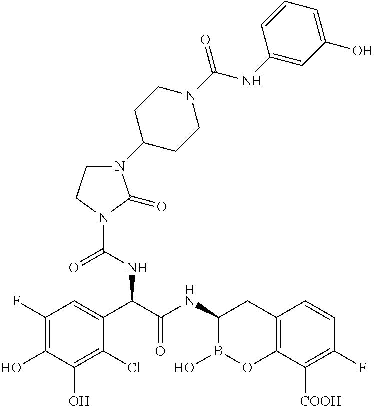 [SMILES: O=C(O)c1c(F)ccc2c1OB(O)[C@@H](NC(=O)[C@H](NC(=O)N1CCN(C3CCN(C(=O)Nc4cccc(O)c4)CC3)C1=O)c1cc(F)c(O)c(O)c1Cl)C2] | (R)-3-((R)-2-(2-chloro-5-fluoro- 3,4-dihydroxyphenyl)-2-(3-(1- ((3- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 772.91 | 773 |
| 446 | 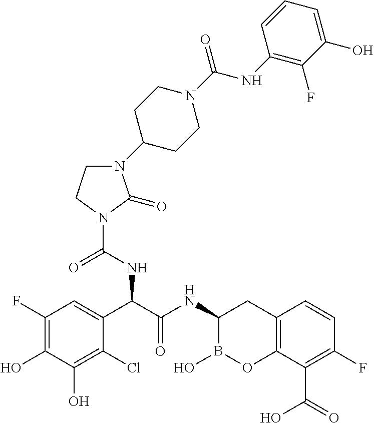 [SMILES: O=C(O)c1c(F)ccc2c1OB(O)[C@@H](NC(=O)[C@H](NC(=O)N1CCN(C3CCN(C(=O)Nc4cccc(O)c4F)CC3)C1=O)c1cc(F)c(O)c(O)c1Cl)C2] | (R)-3-((R)-2-(2-chloro-5-fluoro- 3,4-dihydroxyphenyl)-2-(3-(1- ((2-fluoro-3- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 790.90 | 791 |
| 447 | 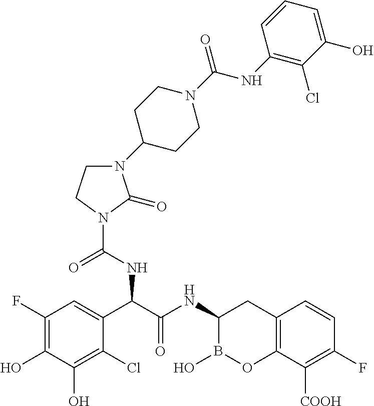 [SMILES: O=C(O)c1c(F)ccc2c1OB(O)[C@@H](NC(=O)[C@H](NC(=O)N1CCN(C3CCN(C(=O)Nc4cccc(O)c4Cl)CC3)C1=O)c1cc(F)c(O)c(O)c1Cl)C2] | (R)-3-((R)-2-(3-(1-((2-chloro-3- hydroxyphenyl)carbamoyl) piperidin-4-yl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 807.35 | 807 |
| 448 | 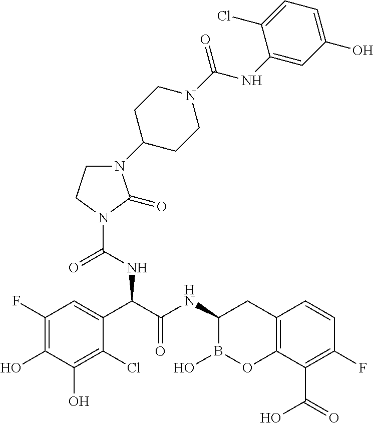 [SMILES: O=C(O)c1c(F)ccc2c1OB(O)[C@@H](NC(=O)[C@H](NC(=O)N1CCN(C3CCN(C(=O)Nc4cc(O)ccc4Cl)CC3)C1=O)c1cc(F)c(O)c(O)c1Cl)C2] | (R)-3-((R)-2-(2-chloro-5-fluoro- 3,4-dihydroxyphenyl)-2-(3-(1- ((2-chloro-5- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 807.35 | 807 |
| 449 | 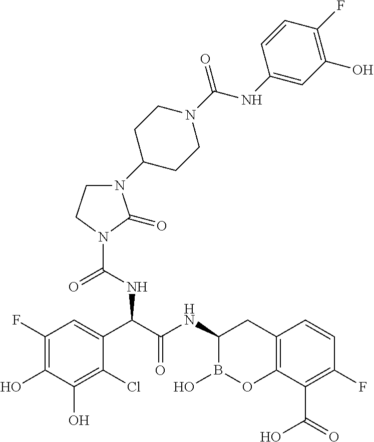 [SMILES: O=C(O)c1c(F)ccc2c1OB(O)[C@@H](NC(=O)[C@H](NC(=O)N1CCN(C3CCN(C(=O)Nc4ccc(F)c(O)c4)CC3)C1=O)c1cc(F)c(O)c(O)c1Cl)C2] | (R)-3-((R)-2-(2-chloro-5-fluoro- 3,4-dihydroxyphenyl)-2-(3-(1- ((4-fluoro-3- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 790.90 | 791 |
| 450 | 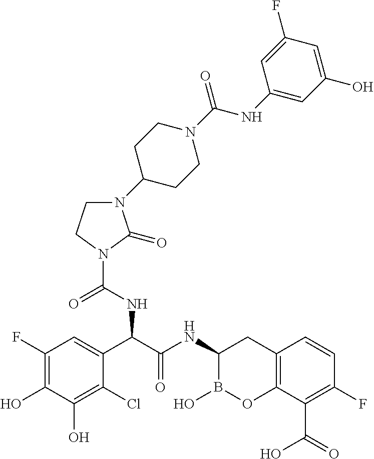 [SMILES: O=C(O)c1c(F)ccc2c1OB(O)[C@@H](NC(=O)[C@H](NC(=O)N1CCN(C3CCN(C(=O)Nc4cc(O)cc(F)c4)CC3)C1=O)c1cc(F)c(O)c(O)c1Cl)C2] | (R)-3-((R)-2-(2-chloro-5-fluoro- 3,4-dihydroxyphenyl)-2-(3-(1- ((3-fluoro-5- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 790.90 | 791 |
| 451 | 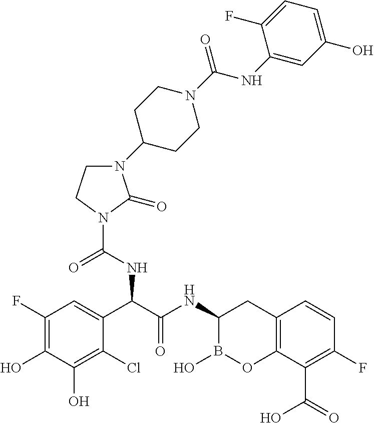 [SMILES: O=C(O)c1c(F)ccc2c1OB(O)[C@@H](NC(=O)[C@H](NC(=O)N1CCN(C3CCN(C(=O)Nc4cc(O)ccc4F)CC3)C1=O)c1cc(F)c(O)c(O)c1Cl)C2] | (R)-3-((R)-2-(2-chloro-5-fluoro- 3,4-dihydroxyphenyl)-2-(3-(1- ((2-fluoro-5- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 790.90 | 791 |
| 452 | 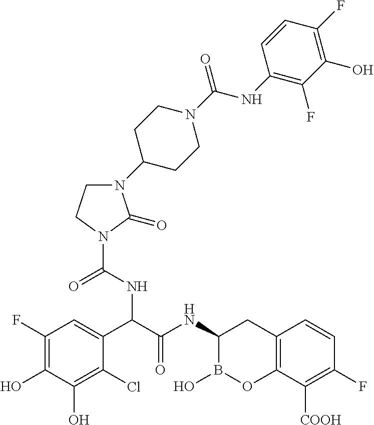 [SMILES: O=C(O)c1c(F)ccc2c1OB(O)[C@@H](NC(=O)C(NC(=O)N1CCN(C3CCN(C(=O)Nc4ccc(F)c(O)c4F)CC3)C1=O)c1cc(F)c(O)c(O)c1Cl)C2] | (3R)-3-(2-(2-chloro-5-fluoro- 3,4-dihydroxyphenyl)-2-(3-(1- ((2,4-difluoro-3- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 808.89 | 809 |
| 454 | 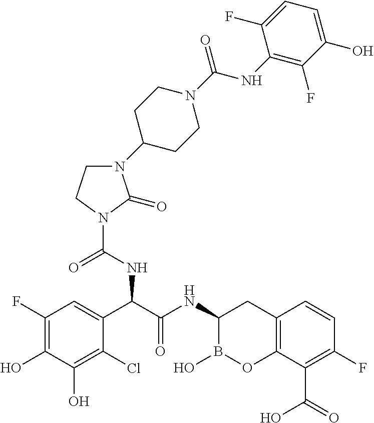 [SMILES: O=C(O)c1c(F)ccc2c1OB(O)[C@@H](NC(=O)[C@H](NC(=O)N1CCN(C3CCN(C(=O)Nc4c(F)ccc(O)c4F)CC3)C1=O)c1cc(F)c(O)c(O)c1Cl)C2] | (R)-3-((R)-2-(2-chloro-5-fluoro- 3,4-dihydroxyphenyl)-2-(3-(1- ((2,6-difluoro-3- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 808.89 | 809 |
| 455 | 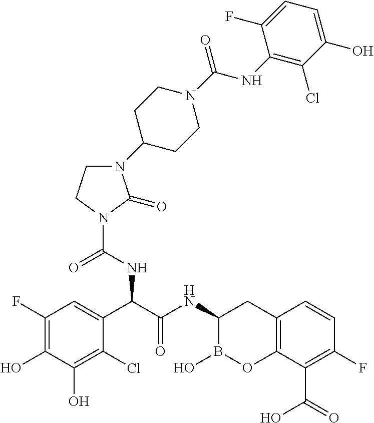 [SMILES: O=C(O)c1c(F)ccc2c1OB(O)[C@@H](NC(=O)[C@H](NC(=O)N1CCN(C3CCN(C(=O)Nc4c(F)ccc(O)c4Cl)CC3)C1=O)c1cc(F)c(O)c(O)c1Cl)C2] | (R)-3-((R)-2-(2-chloro-5-fluoro- 3,4-dihydroxyphenyl)-2-(3-(1- ((2-chloro-6-fluoro-3- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 825.34 | 825 |
| 456 | 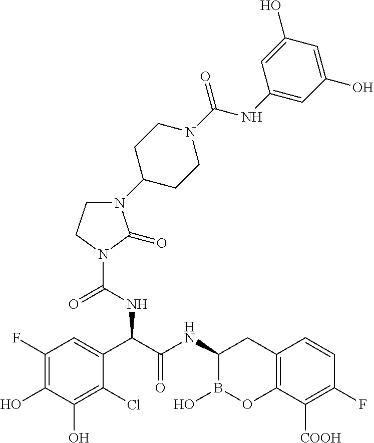 [SMILES: O=C(O)c1c(F)ccc2c1OB(O)[C@@H](NC(=O)[C@H](NC(=O)N1CCN(C3CCN(C(=O)Nc4cc(O)cc(O)c4)CC3)C1=O)c1cc(F)c(O)c(O)c1Cl)C2] | (R)-3-((R)-2-(2-chloro-5-fluoro- 3,4-dihydroxyphenyl)-2-(3-(1- ((3,5- dihydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 788.91 | 789 |
| 459a | 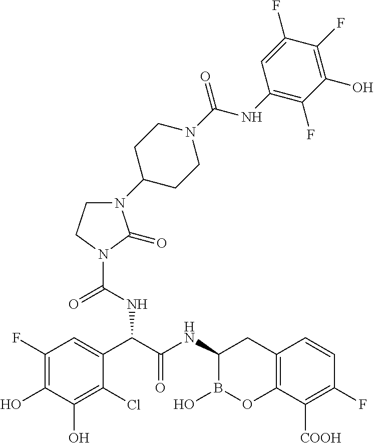 [SMILES: O=C(O)c1c(F)ccc2c1OB(O)[C@@H](NC(=O)[C@@H](NC(=O)N1CCN(C3CCN(C(=O)Nc4cc(F)c(F)c(O)c4F)CC3)C1=O)c1cc(F)c(O)c(O)c1Cl)C2] | (R)-3-((S)-2-(2-chloro-5-fluoro- 3,4-dihydroxyphenyl)-2-(2-oxo- 3-(1-((2,4,5-trifluoro-3- hydroxyphenyl)carbamoyl) piperidin-4-yl) imidazolidine-1- carboxamido)acetamido)-7- fluoro-2-hydroxy-3,4-dihydro- 2H-benzo[e][1,2]oxaborinine-8- carboxylic acid | 826.88 | 827 |
| 459b | 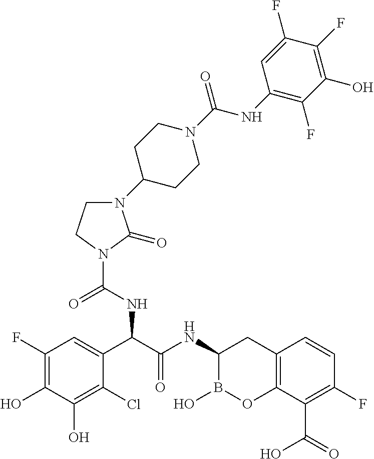 [SMILES: O=C(O)c1c(F)ccc2c1OB(O)[C@@H](NC(=O)[C@H](NC(=O)N1CCN(C3CCN(C(=O)Nc4cc(F)c(F)c(O)c4F)CC3)C1=O)c1cc(F)c(O)c(O)c1Cl)C2] | (R)-3-((R)-2-(2-chloro-5-fluoro- 3,4-dihydroxyphenyl)-2-(2-oxo- 3-(1-((2,4,5-trifluoro-3- hydroxyphenyl)carbamoyl) piperidin-4-yl) imidazolidine-1- carboxamido)acetamido)-7- fluoro-2-hydroxy-3,4-dihydro- 2H-benzo[e][1,2]oxaborinine-8- carboxylic acid | 826.88 | 827 |
| 460 | 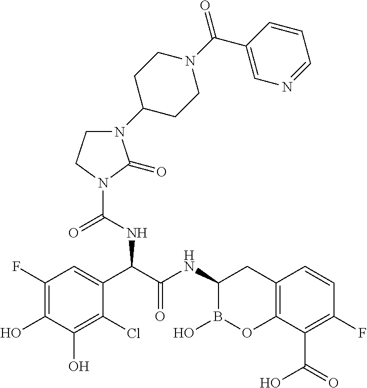 [SMILES: O=C(O)c1c(F)ccc2c1OB(O)[C@@H](NC(=O)[C@H](NC(=O)N1CCN(C3CCN(C(=O)c4cccnc4)CC3)C1=O)c1cc(F)c(O)c(O)c1Cl)C2] | (R)-3-((R)-2-(2-chloro-5-fluoro- 3,4-dihydroxyphenyl)-2-(3-(1- nicotinoylpiperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 742.88 | 743/745 |
| 461 | 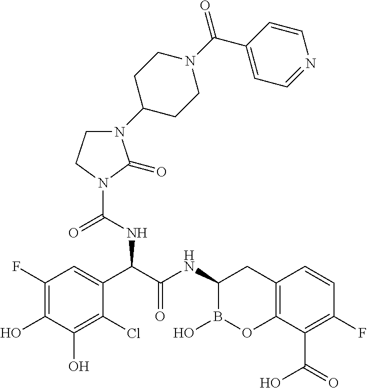 [SMILES: O=C(O)c1c(F)ccc2c1OB(O)[C@@H](NC(=O)[C@H](NC(=O)N1CCN(C3CCN(C(=O)c4ccncc4)CC3)C1=O)c1cc(F)c(O)c(O)c1Cl)C2] | (R)-3-((R)-2-(2-chloro-5-fluoro- 3,4-dihydroxyphenyl)-2-(3-(1- isonicotinoylpiperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 742.88 | 822/824 |
| 462 | 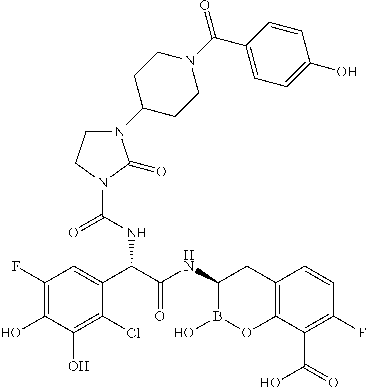 [SMILES: O=C(O)c1c(F)ccc2c1OB(O)[C@@H](NC(=O)[C@@H](NC(=O)N1CCN(C3CCN(C(=O)c4ccc(O)cc4)CC3)C1=O)c1cc(F)c(O)c(O)c1Cl)C2] | (R)-3-((S)-2-(2-chloro-5-fluoro- 3,4-dihydroxyphenyl)-2-(3-(1- (4-hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 757.89 | 758 |
| 463 | 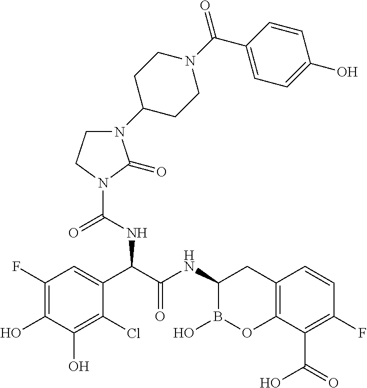 [SMILES: O=C(O)c1c(F)ccc2c1OB(O)[C@@H](NC(=O)[C@H](NC(=O)N1CCN(C3CCN(C(=O)c4ccc(O)cc4)CC3)C1=O)c1cc(F)c(O)c(O)c1Cl)C2] | (R)-3-((R)-2-(2-chloro-5-fluoro- 3,4-dihydroxyphenyl)-2-(3-(1- (4-hydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 757.89 | 758 |
| 464 | 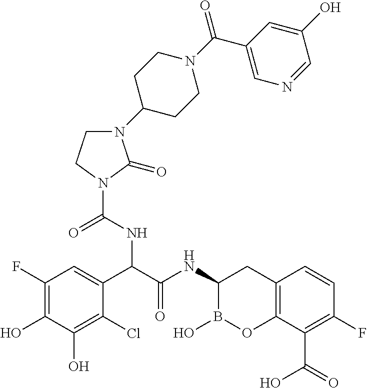 [SMILES: O=C(O)c1c(F)ccc2c1OB(O)[C@@H](NC(=O)C(NC(=O)N1CCN(C3CCN(C(=O)c4cncc(O)c4)CC3)C1=O)c1cc(F)c(O)c(O)c1Cl)C2] | (3R)-3-(2-(2-chloro-5-fluoro- 3,4-dihydroxyphenyl)-2-(3-(1- (5-hydroxynicotinoyl)piperidin- 4-yl)-2-oxoimidazolidine-1- carboxamido)acetamido)-7- fluoro-2-hydroxy-3,4-dihydro- 2H-benzo[e][1,2]oxaborinine-8- carboxylic acid | 758.88 | 759/761 |
| 465 | 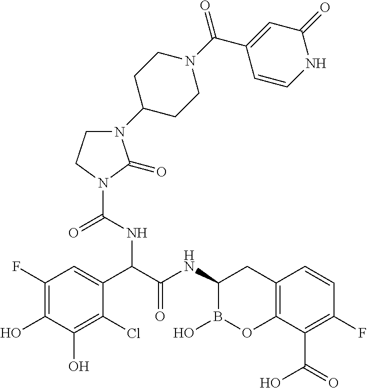 [SMILES: O=C(O)c1c(F)ccc2c1OB(O)[C@@H](NC(=O)C(NC(=O)N1CCN(C3CCN(C(=O)c4cc[nH]c(=O)c4)CC3)C1=O)c1cc(F)c(O)c(O)c1Cl)C2] | (3R)-3-(2-(2-chloro-5-fluoro- 3,4-dihydroxyphenyl)-2-(2-oxo- 3-(1-(2-oxo-1,2- dihydropyridine-4- carbonyl)piperidin-4- yl)imidazolidine-1- carboxamido)acetamido)-7- fluoro-2-hydroxy-3,4-dihydro- 2H-benzo[e][1,2]oxaborinine-8- carboxylic acid | 758.88 | 759/761 |
| 466 | 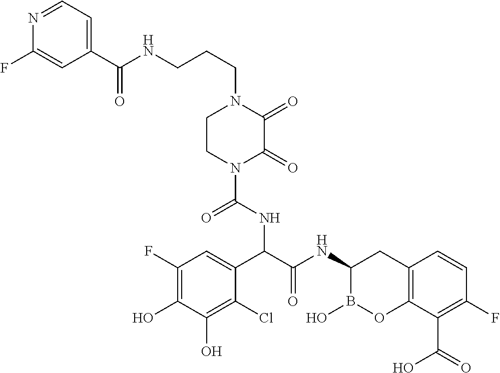 [SMILES: O=C(NCCCN1CCN(C(=O)NC(C(=O)N[C@H]2Cc3ccc(F)c(C(=O)O)c3OB2O)c2cc(F)c(O)c(O)c2Cl)C(=O)C1=O)c1ccnc(F)c1] | (3R)-3-(2-(2-chloro-5-fluoro- 3,4-dihydroxyphenyl)-2-(4-(3- (2- fluoroisonicotinamido)propyl)- 2,3-dioxopiperazine-1- carboxamido)acetamido)-7- fluoro-2-hydroxy-3,4-dihydro- 2H-benzo[e][1,2]oxaborinine-8- carboxylic acid | 762.84 | 763 |
| 467 | 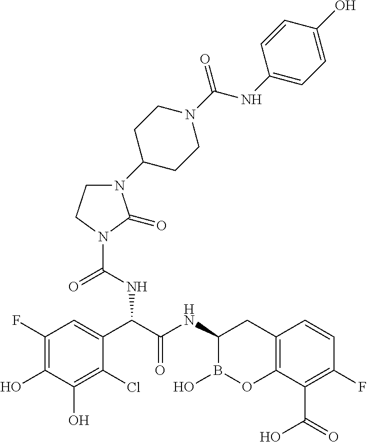 [SMILES: O=C(O)c1c(F)ccc2c1OB(O)[C@@H](NC(=O)[C@@H](NC(=O)N1CCN(C3CCN(C(=O)Nc4ccc(O)cc4)CC3)C1=O)c1cc(F)c(O)c(O)c1Cl)C2] | (R)-3-((S)-2-(2-chloro-5-fluoro- 3,4-dihydroxyphenyl)-2-(3-(1- ((4- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 772.91 | 773 |
| 468 | 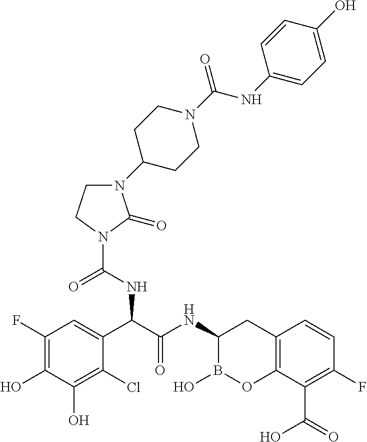 [SMILES: O=C(O)c1c(F)ccc2c1OB(O)[C@@H](NC(=O)[C@H](NC(=O)N1CCN(C3CCN(C(=O)Nc4ccc(O)cc4)CC3)C1=O)c1cc(F)c(O)c(O)c1Cl)C2] | (R)-3-((R)-2-(2-chloro-5-fluoro- 3,4-dihydroxyphenyl)-2-(3-(1- ((4- hydroxyphenyl)carbamoyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 772.91 | 773 |
| 469 | 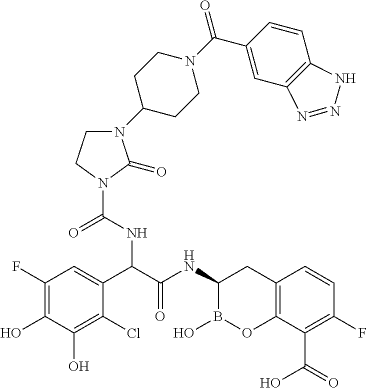 [SMILES: O=C(O)c1c(F)ccc2c1OB(O)[C@@H](NC(=O)C(NC(=O)N1CCN(C3CCN(C(=O)c4ccc5[nH]nnc5c4)CC3)C1=O)c1cc(F)c(O)c(O)c1Cl)C2] | (3R)-3-(2-(3-(1-(1H- benzo[d][1,2,3]triazole-5- carbonyl)piperidin-4-yl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 782.91 | 783/785 |
| 470 | 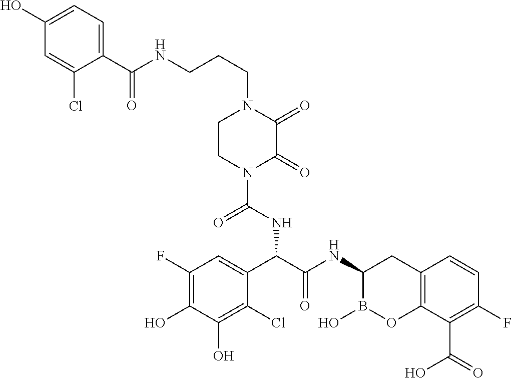 [SMILES: O=C(NCCCN1CCN(C(=O)N[C@H](C(=O)N[C@H]2Cc3ccc(F)c(C(=O)O)c3OB2O)c2cc(F)c(O)c(O)c2Cl)C(=O)C1=O)c1ccc(O)cc1Cl] | (R)-3-((S)-2-(4-(3-(2-chloro-4- hydroxybenzamido)propyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 794.31 | 794 |
| 471 | 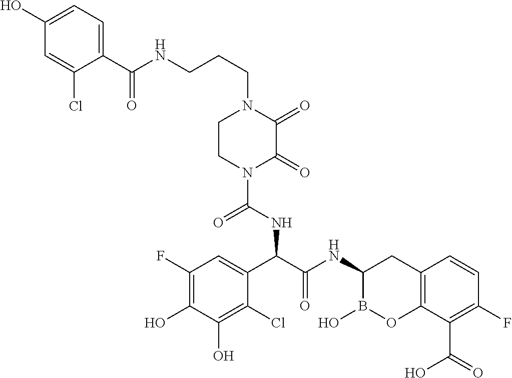 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1ccc(O)cc1Cl] | (R)-3-((R)-2-(4-(3-(2-chloro-4- hydroxybenzamido)propyl)-2,3- dioxopiperazine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 794.31 | 794 |
| 472 | 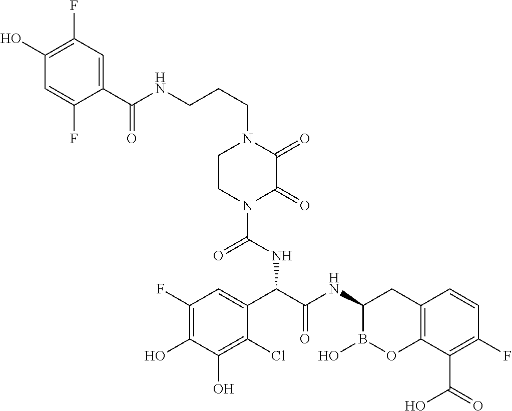 [SMILES: O=C(NCCCN1CCN(C(=O)N[C@H](C(=O)N[C@H]2Cc3ccc(F)c(C(=O)O)c3OB2O)c2cc(F)c(O)c(O)c2Cl)C(=O)C1=O)c1cc(F)c(O)cc1F] | (R)-3-((S)-2-(2-chloro-5-fluoro- 3,4-dihydroxyphenyl)-2-(4-(3- (2,5-difluoro-4- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 795.85 | 796 |
| 473 | 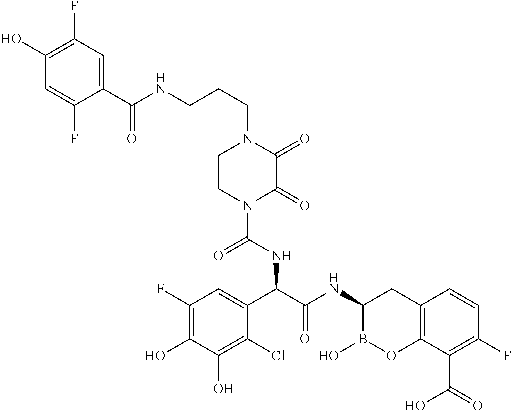 [SMILES: O=C(NCCCN1CCN(C(=O)N[C@@H](C(=O)N[C@H]2Cc3ccc(F)c(C(=O)O)c3OB2O)c2cc(F)c(O)c(O)c2Cl)C(=O)C1=O)c1cc(F)c(O)cc1F] | (R)-3-((R)-2-(2-chloro-5-fluoro- 3,4-dihydroxyphenyl)-2-(4-(3- (2,5-difluoro-4- hydroxybenzamido)propyl)-2,3- dioxopiperazine-1- carboxamido)acetamido)-7- fluoro-2-hydroxy-3,4-dihydro- 2H-benzo[e][1,2]oxaborinine-8- carboxylic acid | 795.85 | 796 |
| 474 | 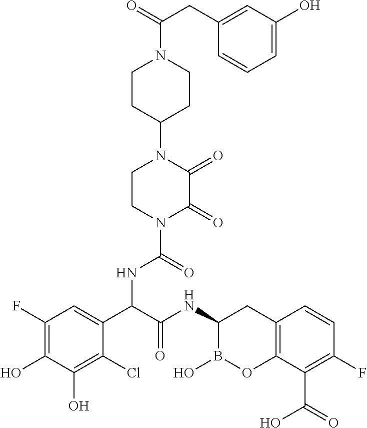 [SMILES: O=C(O)c1c(F)ccc2c1OB(O)[C@@H](NC(=O)C(NC(=O)N1CCN(C3CCN(C(=O)Cc4cccc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- (2-(3- hydroxyphenyl)acetyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 799.93 | 800/802 |
| 475 | 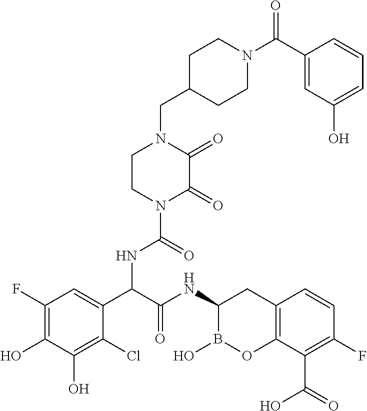 [SMILES: O=C(O)c1c(F)ccc2c1OB(O)[C@@H](NC(=O)C(NC(=O)N1CCN(CC3CCN(C(=O)c4cccc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-((1- (3-hydroxybenzoyl)piperidin- 4-yl)methyl)-2,3-dioxo- piperazine-1-carboxamido) acetamido)-7-fluoro-2- hydroxy-3,4-dihydro- 2H-benzo[e][1,2]oxaborinine-8- carboxylic acid | 799.93 | 800/802 |
| 476 | 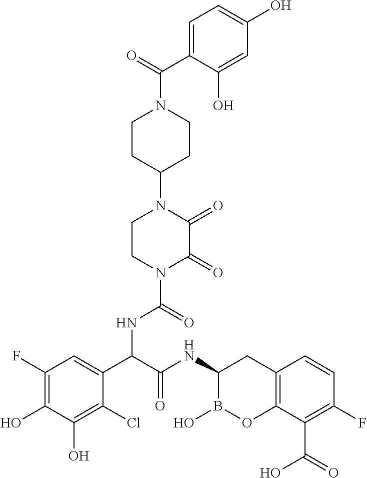 [SMILES: O=C(O)c1c(F)ccc2c1OB(O)[C@@H](NC(=O)C(NC(=O)N1CCN(C3CCN(C(=O)c4ccc(O)cc4O)CC3)C(=O)C1=O)c1cc(F)c(O)c(O)c1Cl)C2] | (3R)-3-(2-(2-chloro-5-fluoro- 3,4-dihydroxyphenyl)-2-(4-(1- (2,4- dihydroxybenzoyl)piperidin-4- yl)-2,3-dioxopiperazine-1- carboxamido)acetamido)-7- fluoro-2-hydroxy-3,4-dihydro- 2H-benzo[e][1,2]oxaborinine-8- carboxylic acid | 801.90 | 802/804 |
| 477 | 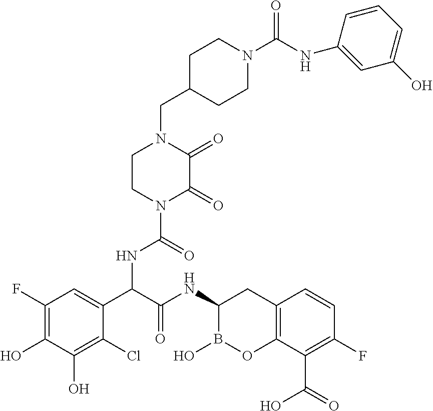 [SMILES: O=C(O)c1c(F)ccc2c1OB(O)[C@@H](NC(=O)C(NC(=O)N1CCN(CC3CCN(C(=O)Nc4cccc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-((1- ((3- hydroxyphenyl)carbamoyl) piperidin-4-yl)methyl)-2,3- dioxopiperazine-1- carboxamido)acetamido)-7- fluoro-2-hydroxy-3,4-dihydro- 2H-benzo[e][1,2]oxaborinine-8- carboxylic acid | 814.94 | 815/817 |
| 478 | 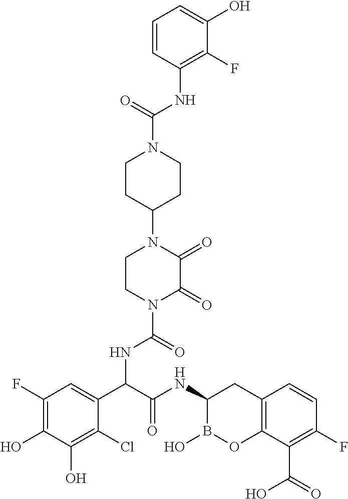 [SMILES: O=C(O)c1c(F)ccc2c1OB(O)[C@@H](NC(=O)C(NC(=O)N1CCN(C3CCN(C(=O)Nc4cccc(O)c4F)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((2-fluoro-3- hydroxyphenyl)carbamoyl) piperidin-4-yl)-2,3-dioxo- piperazine-1-carboxamido) acetamido)-7-fluoro-2- hydroxy-3,4-dihydro- 2H-benzo[e][1,2]oxaborinine-8- carboxylic acid | 818.91 | 819 |
| 479 | 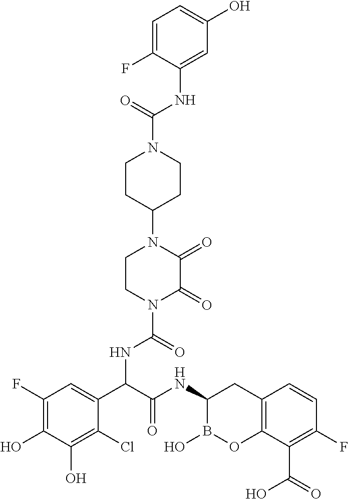 [SMILES: O=C(O)c1c(F)ccc2c1OB(O)[C@@H](NC(=O)C(NC(=O)N1CCN(C3CCN(C(=O)Nc4cc(O)ccc4F)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((2-fluoro-5- hydroxyphenyl)carbamoyl) piperidin-4-yl)-2,3-dioxo- piperazine-1-carboxamido) acetamido)-7-fluoro-2- hydroxy-3,4-dihydro- 2H-benzo[e][1,2]oxaborinine-8- carboxylic acid | 818.91 | 819 |
| 480 | 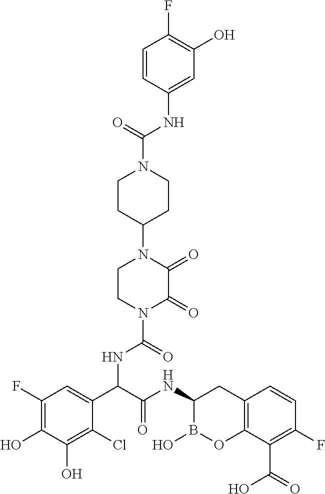 [SMILES: O=C(O)c1c(F)ccc2c1OB(O)[C@@H](NC(=O)C(NC(=O)N1CCN(C3CCN(C(=O)Nc4ccc(F)c(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((4-fluoro-3- hydroxyphenyl)carbamoyl) piperidin-4-yl)-2,3-dioxo- piperazine-1-carboxamido) acetamido)-7- fluoro-2-hydroxy-3,4-dihydro- 2H-benzo[e][1,2]oxaborinine-8- carboxylic acid | 818.91 | 819 |
| 481 | 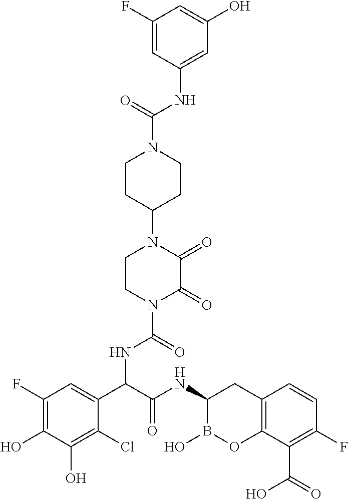 [SMILES: O=C(O)c1c(F)ccc2c1OB(O)[C@@H](NC(=O)C(NC(=O)N1CCN(C3CCN(C(=O)Nc4cc(O)cc(F)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((3-fluoro-5- hydroxyphenyl)carbamoyl) piperidin-4-yl)-2,3-dioxo- piperazine-1-carboxamido) acetamido)-7-fluoro-2- hydroxy-3,4-dihydro- 2H-benzo[e][1,2]oxaborinine-8- carboxylic acid | 818.91 | 819 |
| 482 | 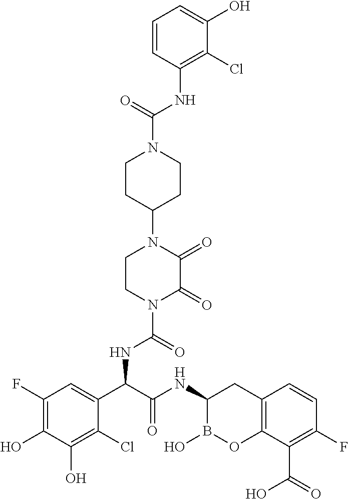 [SMILES: O=C(O)c1c(F)ccc2c1OB(O)[C@@H](NC(=O)[C@H](NC(=O)N1CCN(C3CCN(C(=O)Nc4cccc(O)c4Cl)CC3)C(=O)C1=O)c1cc(F)c(O)c(O)c1Cl)C2] | (R)-3-((R)-2-(4-(1-((2-chloro-3- hydroxyphenyl)carbamoyl) piperidin-4-yl)-2,3-dioxo- piperazine-1-carboxamido)-2- (2-chloro-5-fluoro-3,4- dihydroxyphenyl)acetamido)- 7-fluoro-2-hydroxy- 3,4-dihydro- 2H-benzo[e][1,2]oxaborinine-8- carboxylic acid | 835.36 | 835 |
| 483 | 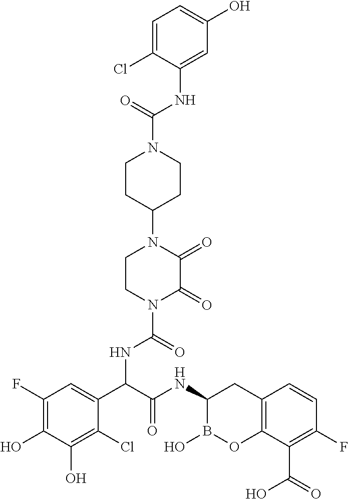 [SMILES: O=C(O)c1c(F)ccc2c1OB(O)[C@@H](NC(=O)C(NC(=O)N1CCN(C3CCN(C(=O)Nc4cc(O)ccc4Cl)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((2-chloro-5- hydroxyphenyl)carbamoyl) piperidin-4-yl)-2,3-dioxo- piperazine-1-carboxamido) acetamido)-7-fluoro-2- hydroxy-3,4-dihydro- 2H-benzo[e][1,2]oxaborinine-8- carboxylic acid | 835.36 | 835 |
| 484 | 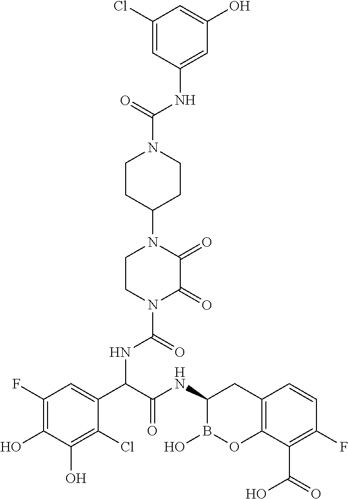 [SMILES: O=C(O)c1c(F)ccc2c1OB(O)[C@@H](NC(=O)C(NC(=O)N1CCN(C3CCN(C(=O)Nc4cc(O)cc(Cl)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((3-chloro-5- hydroxyphenyl)carbamoyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 835.36 | 835 |
| 485 | 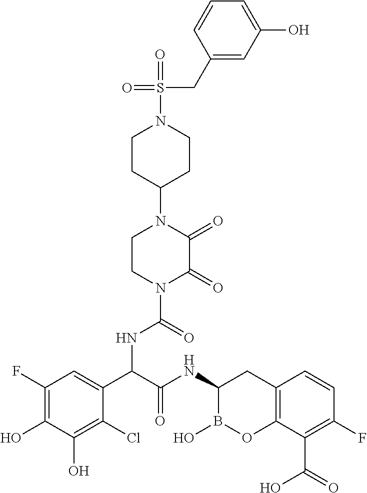 [SMILES: O=C(O)c1c(F)ccc2c1OB(O)[C@@H](NC(=O)C(NC(=O)N1CCN(C3CCN(S(=O)(=O)Cc4cccc(O)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((3- hydroxybenzyl)sulfonyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 835.98 | 836/838 |
| 486 | 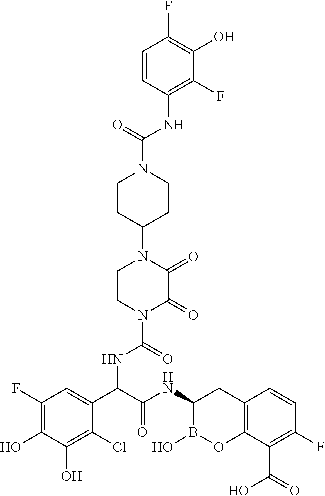 [SMILES: O=C(O)c1c(F)ccc2c1OB(O)[C@@H](NC(=O)C(NC(=O)N1CCN(C3CCN(C(=O)Nc4ccc(F)c(O)c4F)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((2,4-difluoro-3- hydroxyphenyl)carbamoyl) piperidin-4-yl)-2,3- dioxopiperazine-1- carboxamido)acetamido)-7- fluoro-2-hydroxy-3,4-dihydro- 2H-benzo[e][1,2]oxaborinine-8- carboxylic acid | 836.90 | 837 |
| 487 | 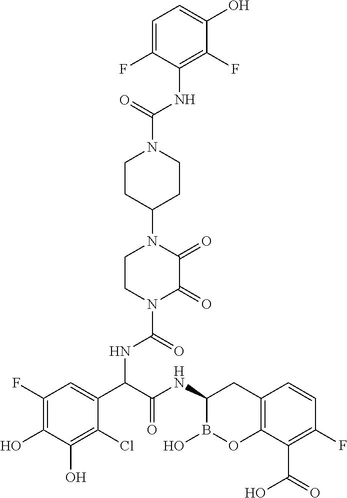 [SMILES: O=C(O)c1c(F)ccc2c1OB(O)[C@@H](NC(=O)C(NC(=O)N1CCN(C3CCN(C(=O)Nc4c(F)ccc(O)c4F)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((2,6-difluoro-3- hydroxyphenyl)carbamoyl) piperidin-4-yl)-3,4-dioxo- piperazine-1-carboxamido) acetamido)-7-fluoro-2- hydroxy-3,4-dihydro- 2H-benzo[e][1,2]oxaborinine-8- carboxylic acid | 836.90 | 837 |
| 488 | 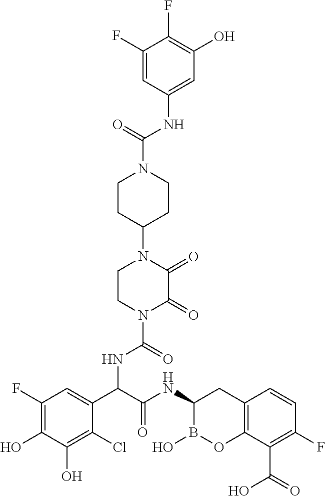 [SMILES: O=C(O)c1c(F)ccc2c1OB(O)[C@@H](NC(=O)C(NC(=O)N1CCN(C3CCN(C(=O)Nc4cc(O)c(F)c(F)c4)CC3)C(=O)C1=O)c1cc(F)c(O)c(O)c1Cl)C2] | (3R)-3-(2-(2-chloro-5-fluoro- 3,4-dihydroxyphenyl)-2-(4-(1- ((3,4-difluoro-5- hydroxyphenyl)carbamoyl) piperidin-4-yl)-2,3-dioxo- piperazine-1-carboxamido) acetamido)-7-fluoro-2- hydroxy-3,4-dihydro- 2H-benzo[e][1,2]oxaborinine-8- carboxylic acid | 836.90 | 837 |
| 489 | 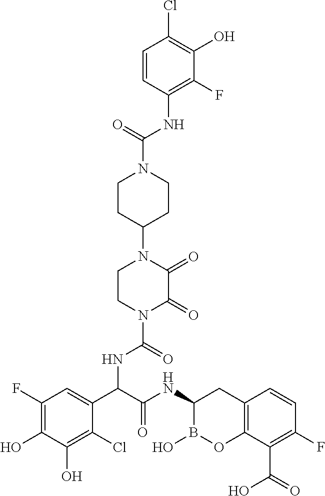 [SMILES: O=C(O)c1c(F)ccc2c1OB(O)[C@@H](NC(=O)C(NC(=O)N1CCN(C3CCN(C(=O)Nc4ccc(Cl)c(O)c4F)CC3)C(=O)C1=O)c1cc(F)c(O)c(O)c1Cl)C2] | (3R)-3-(2-(4-(1-((4-chloro-2- fluoro-3- hydroxyphenyl)carbamoyl) piperidin-4-yl)-2,3- dioxopiperazine-1- carboxamido)-2-(2-chloro-5- fluoro-3,4- dihydroxyphenyl)acetamido)- 7-fluoro-2-hydroxy-3,4- dihydro-2H-benzo [e][1,2]oxaborinine-8- carboxylic acid | 853.35 | 853 |
| 490 | 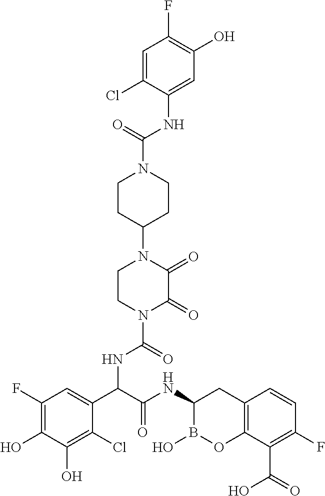 [SMILES: O=C(O)c1c(F)ccc2c1OB(O)[C@@H](NC(=O)C(NC(=O)N1CCN(C3CCN(C(=O)Nc4cc(O)c(F)cc4Cl)CC3)C(=O)C1=O)c1cc(F)c(O)c(O)c1Cl)C2] | (3R)-3-(2-(4-(1-((2-chloro-4- fluoro-5- hydroxyphenyl)carbamoyl) piperidin-4-yl)-2,3- dioxopiperazine-1- carboxamido)-2-(2-chloro-5- fluoro-3,4- dihydroxyphenyl)acetamido)- 7-fluoro-2-hydroxy- 3,4-dihydro- 2H-benzo[e][1,2]oxaborinine-8- carboxylic acid | 853.35 | 853 |
| 491 | 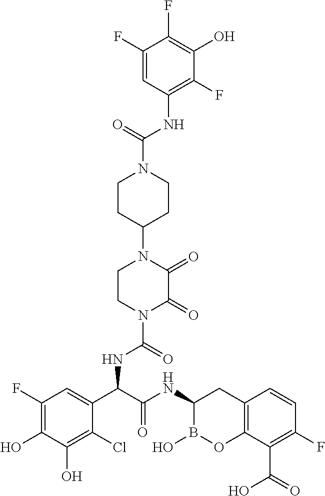 [SMILES: O=C(O)c1c(F)ccc2c1OB(O)[C@@H](NC(=O)[C@H](NC(=O)N1CCN(C3CCN(C(=O)Nc4cc(F)c(F)c(O)c4F)CC3)C(=O)C1=O)c1cc(F)c(O)c(O)c1Cl)C2] | (R)-3-((R)-2-(2-chloro-5-fluoro- 3,4-dihydroxyphenyl)-2-(2,3- dioxo-4-(1-((2,4,5-trifluoro-3- hydroxyphenyl)carbamoyl) piperidin-4-yl)piperazine-1- carboxamido)acetamido)-7- fluoro-2-hydroxy-3,4-dihydro- 2H-benzo[e][1,2]oxaborinine-8- carboxylic acid | 854.89 | 855 |
| 492 | 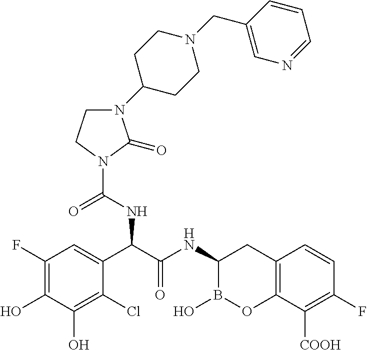 [SMILES: O=C(O)c1c(F)ccc2c1OB(O)[C@@H](NC(=O)[C@H](NC(=O)N1CCN(C3CCN(Cc4cccnc4)CC3)C1=O)c1cc(F)c(O)c(O)c1Cl)C2] | (R)-3-((R)-2-(2-chloro-5-fluoro- 3,4-dihydroxyphenyl)-2-(2- oxo-3-(1-(pyridin-3- ylmethyl)piperidin-4- yl)imidazolidine-1- carboxamido)acetamido)-7- fluoro-2-hydroxy-3,4-dihydro- 2H-benzo[e][1,2]oxaborinine-8- carboxylic acid | 728.90 | 729/731 |
| 493 | 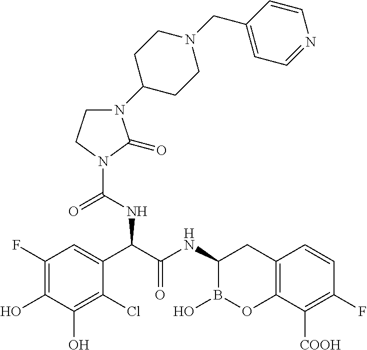 [SMILES: O=C(O)c1c(F)ccc2c1OB(O)[C@@H](NC(=O)[C@H](NC(=O)N1CCN(C3CCN(Cc4ccncc4)CC3)C1=O)c1cc(F)c(O)c(O)c1Cl)C2] | (R)-3-((R)-2-(2-chloro-5-fluoro- 3,4-dihydroxyphenyl)-2-(2- oxo-3-(1-(pyridin-4- ylmethyl)piperidin-4- yl)imidazolidine-1- carboxamido)acetamido)-7- fluoro-2-hydroxy-3,4-dihydro- 2H-benzo[e][1,2]oxaborinine-8- carboxylic acid | 728.90 | 729/731 |
| 600 | 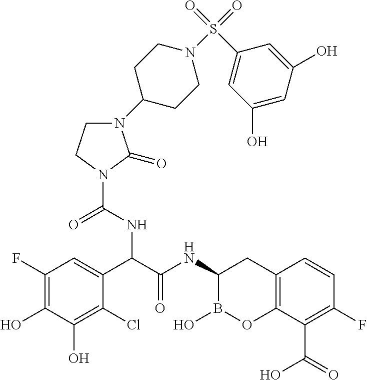 [SMILES: O=C(O)c1c(F)ccc2c1OB(O)[C@@H](NC(=O)C(NC(=O)N1CCN(C3CCN(S(=O)(=O)c4cc(O)cc(O)c4)CC3)C1=O)c1cc(F)c(O)c(O)c1Cl)C2] | (3R)-3-(2-(2-chloro-5-fluoro- 3,4-dihydroxyphenyl)-2-(3-(1- ((3,5- dihydroxyphenyl)sulfonyl) piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 809.94 | 810/812 |
| 601 | 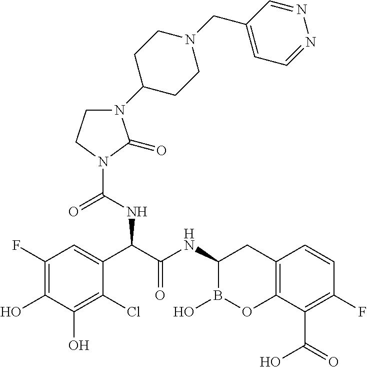 [SMILES: O=C(O)c1c(F)ccc2c1OB(O)[C@@H](NC(=O)[C@H](NC(=O)N1CCN(C3CCN(Cc4ccnnc4)CC3)C1=O)c1cc(F)c(O)c(O)c1Cl)C2] | (R)-3-((R)-2-(2-chloro-5-fluoro- 3,4-dihydroxyphenyl)-2-(2- oxo-3-(1-(pyridazin-4- ylmethyl)piperidin-4- yl)imidazolidine-1- carboxamido)acetamido)-7- fluoro-2-hydroxy-3,4-dihydro- 2H-benzo[e][1,2]oxaborinine-8- carboxylic acid | 729.89 | 730/732 |
| 602 | 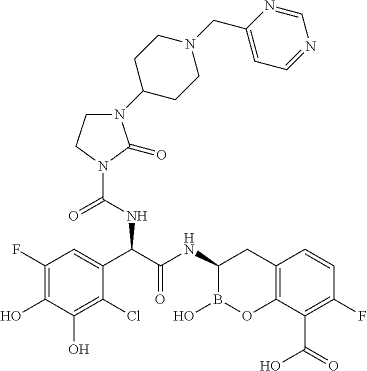 [SMILES: O=C(O)c1c(F)ccc2c1OB(O)[C@@H](NC(=O)[C@H](NC(=O)N1CCN(C3CCN(Cc4ccncn4)CC3)C1=O)c1cc(F)c(O)c(O)c1Cl)C2] | (R)-3-((R)-2-(2-chloro-5-fluoro- 3,4-dihydroxyphenyl)-2-(2- oxo-3-(1-(pyrimidin-4- ylmethyl)piperidin-4- yl)imidazolidine-1- carboxamido)acetamido)-7- fluoro-2-hydroxy-3,4-dihydro- 2H-benzo[e][1,2]oxaborinine-8- carboxylic acid | 729.89 | 730/732 |
| 603 | 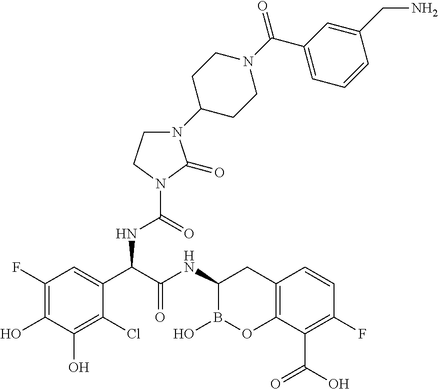 [SMILES: NCc1cccc(C(=O)N2CCC(N3CCN(C(=O)N[C@@H](C(=O)N[C@H]4Cc5ccc(F)c(C(=O)O)c5OB4O)c4cc(F)c(O)c(O)c4Cl)C3=O)CC2)c1] | (R)-3-((R)-2-(3-(1-(3- (aminomethyl)benzoyl) piperidin-4-yl)-2-oxo- imidazolidine-1-carboxamido)- 2-(2-chloro-5-fluoro-3,4- dihydroxyphenyl)acetamido)-7- fluoro-2-hydroxy-3,4-dihydro- 2H-benzo[e][1,2]oxaborinine-8- carboxylic acid | 770.93 | 771 |
| 604 | 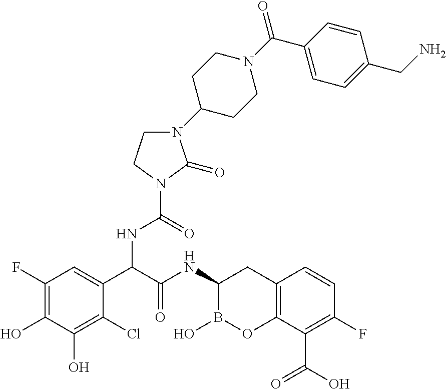 [SMILES: NCc1ccc(C(=O)N2CCC(N3CCN(C(=O)NC(C(=O)N[C@H]4Cc5ccc(F)c(C(=O)O)c5OB4O)c4cc(F)c(O)c(O)c4Cl)C3=O)CC2)cc1] | (3R)-3-(2-(3-(1-(4- (aminomethyl)benzoyl) piperidin-4-yl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 770.93 | 771 |
| 605 | 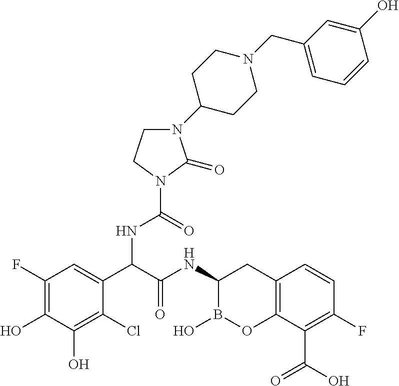 [SMILES: O=C(O)c1c(F)ccc2c1OB(O)[C@@H](NC(=O)C(NC(=O)N1CCN(C3CCN(Cc4cccc(O)c4)CC3)C1=O)c1cc(F)c(O)c(O)c1Cl)C2] | (3R)-3-(2-(2-chloro-5-fluoro- 3,4-dihydroxyphenyl)-2-(3-(1- (3-hydroxybenzyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 743.91 | 744 |
| 606 | 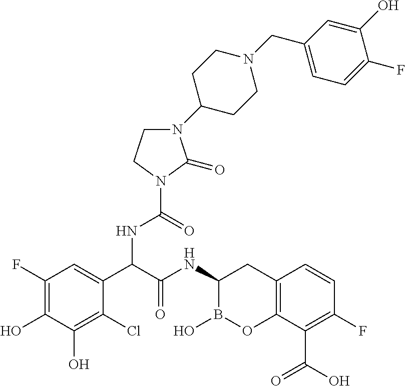 [SMILES: O=C(O)c1c(F)ccc2c1OB(O)[C@@H](NC(=O)C(NC(=O)N1CCN(C3CCN(Cc4ccc(F)c(O)c4)CC3)C1=O)c1cc(F)c(O)c(O)c1Cl)C2] | (3R)-3-(2-(2-chloro-5-fluoro- 3,4-dihydroxyphenyl)-2-(3-(1- (4-fluoro-3- hydroxybenzyl)piperidin-4-yl)- 2-oxoimidazolidine-1- carboxamido)acetamido)-7- fluoro-2-hydroxy-3,4-dihydro- 2H-benzo[e][1,2]oxaborinine-8- carboxylic acid | 761.90 | 762 |
| 607 | 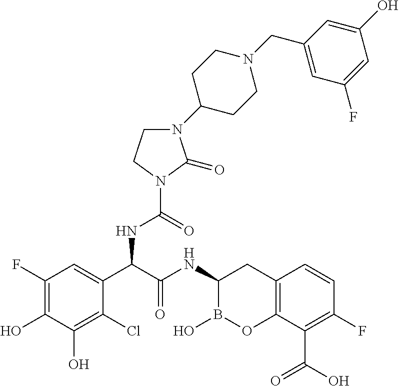 [SMILES: O=C(O)c1c(F)ccc2c1OB(O)[C@@H](NC(=O)[C@H](NC(=O)N1CCN(C3CCN(Cc4cc(O)cc(F)c4)CC3)C1=O)c1cc(F)c(O)c(O)c1Cl)C2] | (R)-3-((R)-2-(2-chloro-5-fluoro- 3,4-dihydroxyphenyl)-2-(3-(1- (3-fluoro-5- hydroxybenzyl)piperidin-4-yl)- 2-oxoimidazolidine-1- carboxamido)acetamido)-7- fluoro-2-hydroxy-3,4-dihydro- 2H-benzo[e][1,2]oxaborinine-8- carboxylic acid | 761.90 | 762 |
| 608 | 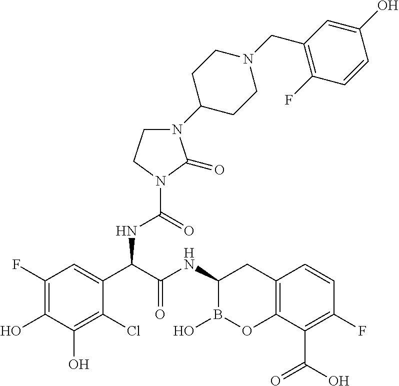 [SMILES: O=C(O)c1c(F)ccc2c1OB(O)[C@@H](NC(=O)[C@H](NC(=O)N1CCN(C3CCN(Cc4cc(O)ccc4F)CC3)C1=O)c1cc(F)c(O)c(O)c1Cl)C2] | (R)-3-((R)-2-(2-chloro-5-fluoro- 3,4-dihydroxyphenyl)-2-(3-(1- (2-fluoro-5- hydroxybenzyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 761.90 | 762 |
| 609 | 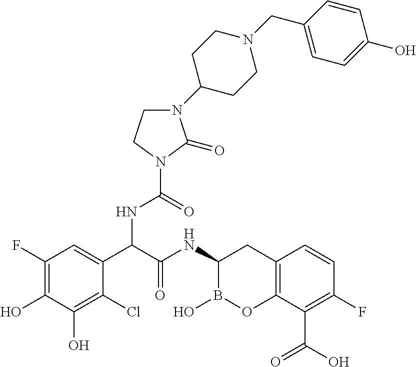 [SMILES: O=C(O)c1c(F)ccc2c1OB(O)[C@@H](NC(=O)C(NC(=O)N1CCN(C3CCN(Cc4ccc(O)cc4)CC3)C1=O)c1cc(F)c(O)c(O)c1Cl)C2] | (3R)-3-(2-(2-chloro-5-fluoro- 3,4-dihydroxyphenyl)-2-(3-(1- (4-hydroxybenzyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 743.91 | 744 |
| 610 | 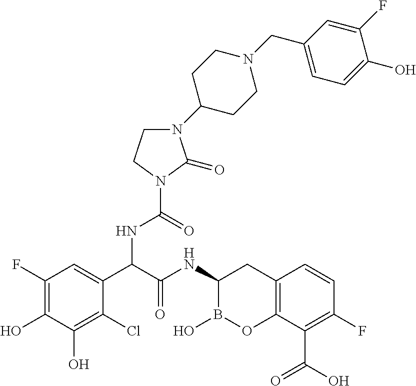 [SMILES: O=C(O)c1c(F)ccc2c1OB(O)[C@@H](NC(=O)C(NC(=O)N1CCN(C3CCN(Cc4ccc(O)c(F)c4)CC3)C1=O)c1cc(F)c(O)c(O)c1Cl)C2] | (3R)-3-(2-(2-chloro-5-fluoro- 3,4-dihydroxyphenyl)-2-(3-(1- (3-fluoro-4- hydroxybenzyl)piperidin-4-yl)- 2-oxoimidazolidine-1- carboxamido)acetamido)-7- fluoro-2-hydroxy-3,4-dihydro- 2H-benzo[e][1,2]oxaborinine-8- carboxylic acid | 761.90 | 762 |
| 611 | 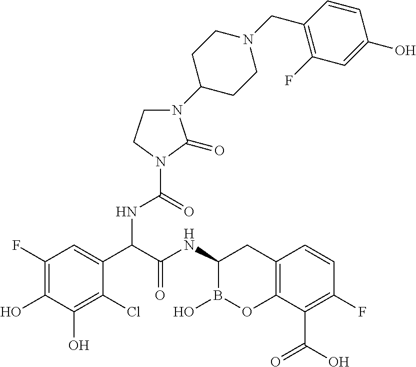 [SMILES: O=C(O)c1c(F)ccc2c1OB(O)[C@@H](NC(=O)C(NC(=O)N1CCN(C3CCN(Cc4ccc(O)cc4F)CC3)C1=O)c1cc(F)c(O)c(O)c1Cl)C2] | (3R)-3-(2-(2-chloro-5-fluoro- 3,4-dihydroxyphenyl)-2-(3-(1- (2-fluoro-4- hydroxybenzyl)piperidin-4-yl)- 2-oxoimidazolidine-1- carboxamido)acetamido)-7- fluoro-2-hydroxy-3,4-dihydro- 2H-benzo[e][1,2]oxaborinine-8- carboxylic acid | 761.90 | 762 |
| 612 | 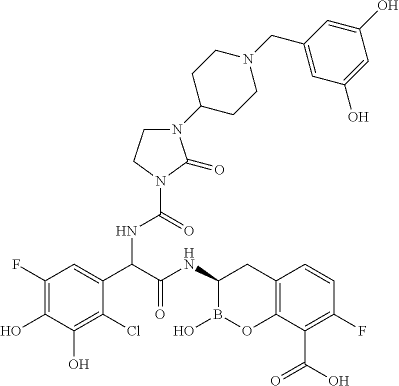 [SMILES: O=C(O)c1c(F)ccc2c1OB(O)[C@@H](NC(=O)C(NC(=O)N1CCN(C3CCN(Cc4cc(O)cc(O)c4)CC3)C1=O)c1cc(F)c(O)c(O)c1Cl)C2] | (3R)-3-(2-(2-chloro-5-fluoro- 3,4-dihydroxyphenyl)-2-(3-(1- (3,5-dihydroxybenzyl)piperidin- 4-yl)-2-oxoimidazolidine-1- carboxamido)acetamido)-7- fluoro-2-hydroxy-3,4-dihydro- 2H-benzo[e][1,2]oxaborinine-8- carboxylic acid | 759.91 | 760 |
| 613 | 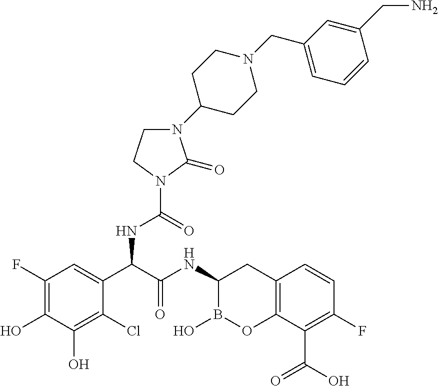 [SMILES: NCc1cccc(CN2CCC(N3CCN(C(=O)N[C@@H](C(=O)N[C@H]4Cc5ccc(F)c(C(=O)O)c5OB4O)c4cc(F)c(O)c(O)c4Cl)C3=O)CC2)c1] | (R)-3-((R)-2-(3-(1-(3- (aminomethyl)benzyl)piperidin- 4-yl)-2-oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 756.95 | 757 |
| 614 | 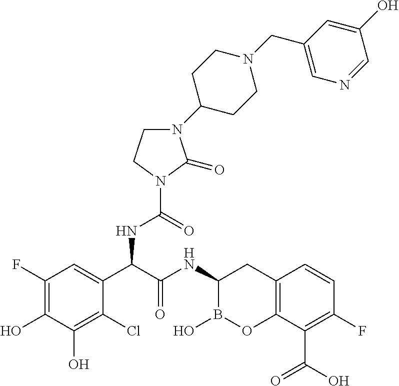 [SMILES: O=C(O)c1c(F)ccc2c1OB(O)[C@@H](NC(=O)[C@H](NC(=O)N1CCN(C3CCN(Cc4cncc(O)c4)CC3)C1=O)c1cc(F)c(O)c(O)c1Cl)C2] | (R)-3-((R)-2-(2-chloro-5-fluoro- 3,4-dihydroxyphenyl)-2-(3-(1- ((5-hydroxypyridin-3- yl)methyl)piperidin-4-yl)-2- oxoimidazolidine-1- carboxamido)acetamido)-7- fluoro-2-hydroxy-3,4-dihydro- 2H-benzo[e][1,2]oxaborinine-8- carboxylic acid | 744.90 | 745 |
| 615 | 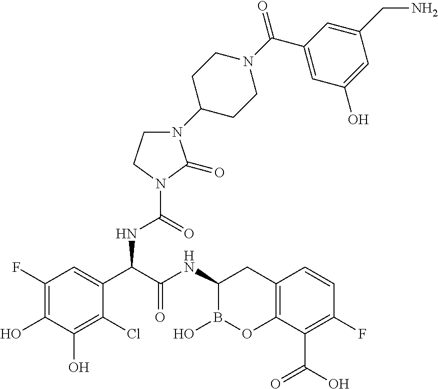 [SMILES: NCc1cc(O)cc(C(=O)N2CCC(N3CCN(C(=O)N[C@@H](C(=O)N[C@H]4Cc5ccc(F)c(C(=O)O)c5OB4O)c4cc(F)c(O)c(O)c4Cl)C3=O)CC2)c1] | (R)-3-((R)-2-(3-(1-(3- (aminomethyl)-5- hydroxybenzoyl)piperidin-4- yl)-2-oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 786.93 | 787 |
| 616 | 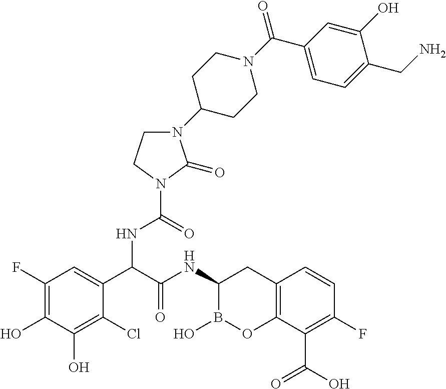 [SMILES: NCc1ccc(C(=O)N2CCC(N3CCN(C(=O)NC(C(=O)N[C@H]4Cc5ccc(F)c(C(=O)O)c5OB4O)c4cc(F)c(O)c(O)c4Cl)C3=O)CC2)cc1O] | (3R)-3-(2-(3-(1-(4- (aminomethyl)-3- hydroxybenzoyl)piperidin-4- yl)-2-oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 786.93 | 787 |
| 617 | 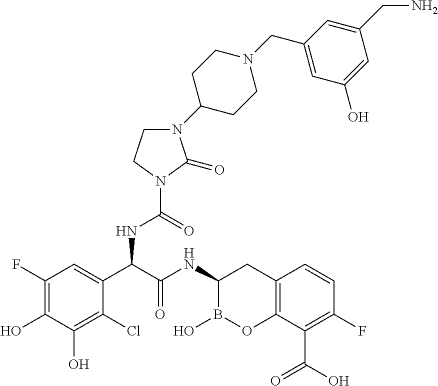 [SMILES: NCc1cc(O)cc(CN2CCC(N3CCN(C(=O)N[C@@H](C(=O)N[C@H]4Cc5ccc(F)c(C(=O)O)c5OB4O)c4cc(F)c(O)c(O)c4Cl)C3=O)CC2)c1] | (R)-3-((R)-2-(3-(1-(3- (aminomethyl)-5- hydroxybenzyl)piperidin-4-yl)- 2-oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 772.95 | 773 |
| 618 | 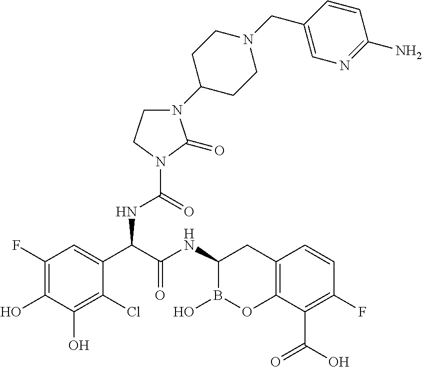 [SMILES: Nc1ccc(CN2CCC(N3CCN(C(=O)N[C@@H](C(=O)N[C@H]4Cc5ccc(F)c(C(=O)O)c5OB4O)c4cc(F)c(O)c(O)c4Cl)C3=O)CC2)cn1] | (R)-3-((R)-2-(3-(1-((6- aminopyridin-3- yl)methyl)piperidin-4-yl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 743.91 | 744 |
| 619 | 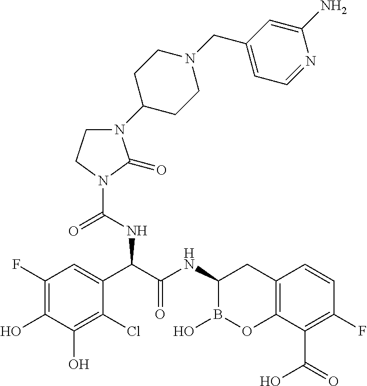 [SMILES: Nc1cc(CN2CCC(N3CCN(C(=O)N[C@@H](C(=O)N[C@H]4Cc5ccc(F)c(C(=O)O)c5OB4O)c4cc(F)c(O)c(O)c4Cl)C3=O)CC2)ccn1] | (R)-3-((R)-2-(3-(1-((2- aminopyridin-4- yl)methyl)piperidin-4-yl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 743.91 | 744/746 |
| 620 | 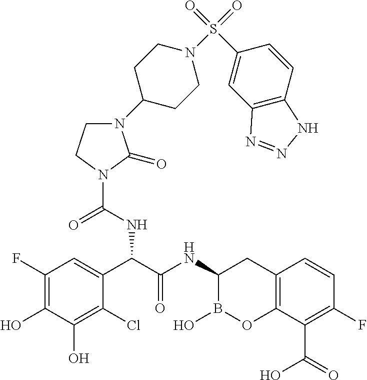 [SMILES: O=C(O)c1c(F)ccc2c1OB(O)[C@@H](NC(=O)[C@@H](NC(=O)N1CCN(C3CCN(S(=O)(=O)c4ccc5[nH]nnc5c4)CC3)C1=O)c1cc(F)c(O)c(O)c1Cl)C2] | (R)-3-((S)-2-(3-(1-((1H- benzo[d][1,2,3]triazol-5- yl)sulfonyl)piperidin-4-yl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 818.95 | 819/821 |
| 621 | 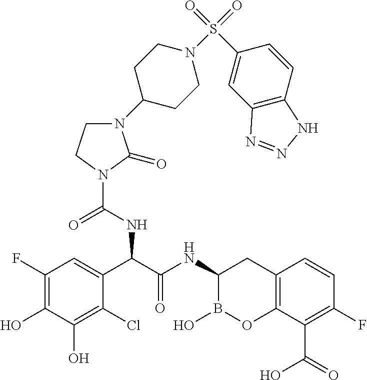 [SMILES: O=C(O)c1c(F)ccc2c1OB(O)[C@@H](NC(=O)[C@H](NC(=O)N1CCN(C3CCN(S(=O)(=O)c4ccc5[nH]nnc5c4)CC3)C1=O)c1cc(F)c(O)c(O)c1Cl)C2] | (R)-3-((R)-2-(3-(1-((1H- benzo[d][1,2,3]triazol-5- yl)sulfonyl)piperidin-4-yl)-2- oxoimidazolidine-1- carboxamido)-2-(2-chloro-5- fluoro-3,4- dihydroxyphenyl)acetamido)-7- fluoro-2-hydroxy-3,4-dihydro- 2H-benzo[e][1,2]oxaborinine-8- carboxylic acid | 818.95 | 819/821 |
| 622 | 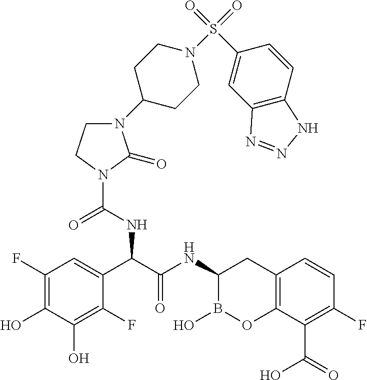 [SMILES: O=C(O)c1c(F)ccc2c1OB(O)[C@@H](NC(=O)[C@H](NC(=O)N1CCN(C3CCN(S(=O)(=O)c4ccc5[nH]nnc5c4)CC3)C1=O)c1cc(F)c(O)c(O)c1F)C2] | (R)-3-((R)-2-(3-(1-((1H- benzo[d][1,2,3]triazol-5- yl)sulfonyl)piperidin-4-yl)-2- oxoimidazolidine-1- carboxamido)-2-(2,5-difluoro- 3,4- dihydroxyphenyl)acetamido)-7- fluoro-2-hydroxy-3,4-dihydro- 2H-benzo[e][1,2]oxaborinine-8- carboxylic acid | 802.50 | 803/805 |
| 623 | 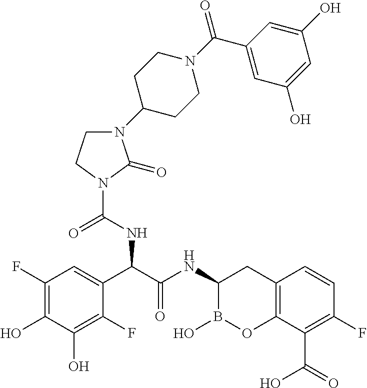 [SMILES: O=C(O)c1c(F)ccc2c1OB(O)[C@@H](NC(=O)[C@H](NC(=O)N1CCN(C3CCN(C(=O)c4cc(O)cc(O)c4)CC3)C1=O)c1cc(F)c(O)c(O)c1F)C2] | (R)-3-((R)-2-(2,5-difluoro-3,4- dihydroxyphenyl)-2-(3-(1-(3,5- dihydroxybenzoyl)piperidin-4- yl)-2-oxoimidazolidine-1- carboxamido)acetamido)-7- fluoro-2-hydroxy-3,4-dihydro- 2H-benzo[e][1,2]oxaborinine-8- carboxylic acid | 757.44 | 758/760 |

TABLE 4

| Ex. | Structure | Name | MW |
|---|---|---|---|
| 453 | 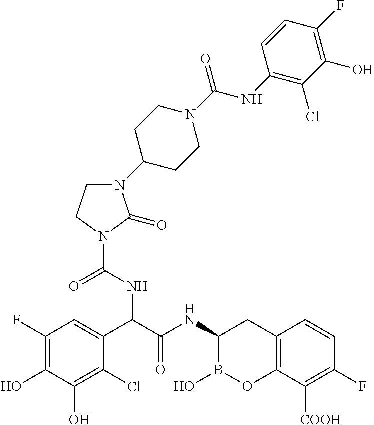 [SMILES: O=C(O)c1c(F)ccc2c1OB(O)[C@@H](NC(=O)C(NC(=O)N1CCN(C3CCN(C(=O)Nc4ccc(F)c(O)c4Cl)CC3)C1=O)c1cc(F)c(O)c(O)c1Cl)C2] | (3R)-3-(2-(3-(1-((2-chloro-4-fluoro-3- hydroxyphenyl)carbamoyl)piperidin-4- yl)-2-oxoimidazolidine-1- carboxamido)-2-(2-chloro-5-fluoro-3,4- dihydroxyphenyl)acetamido)-7-fluoro- 2-hydroxy-3,4-dihydro-2H- benzo[e][1,2]oxaborinine-8-carboxylic acid | 825.34 |
| 457 | 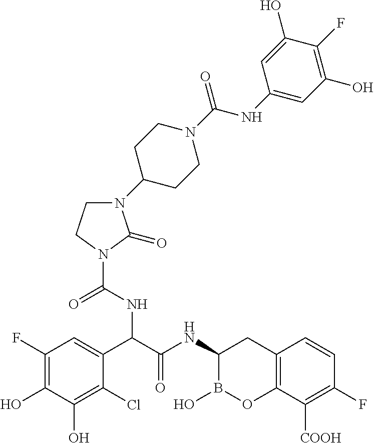 [SMILES: O=C(O)c1c(F)ccc2c1OB(O)[C@@H](NC(=O)C(NC(=O)N1CCN(C3CCN(C(=O)Nc4cc(O)c(F)c(O)c4)CC3)C1=O)c1cc(F)c(O)c(O)c1Cl)C2] | (3R)-3-(2-(2-chloro-5-fluoro-3,4- dihydroxyphenyl)-2-(3-(1-((4-fluoro- 3,5- dihydroxyphenyl)carbamoyl)piperidin- 4-yl)-2-oxoimidazolidine-1- carboxamido)acetamido)-7-fluoro-2- hydroxy-3,4-dihydro-2H- benzo[e][1,2]oxaborinine-8-carboxylic acid | 806.90 |
| 458 | 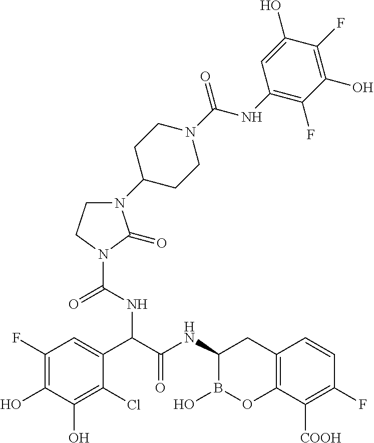 [SMILES: O=C(O)c1c(F)ccc2c1OB(O)[C@@H](NC(=O)C(NC(=O)N1CCN(C3CCN(C(=O)Nc4cc(O)c(F)c(O)c4F)CC3)C1=O)c1cc(F)c(O)c(O)c1Cl)C2] | (3R)-3-(2-(2-chloro-5-fluoro-3,4- dihydroxyphenyl)-2-(3-(1-((2,4- difluoro-3,5- dihydroxyphenyl)carbamoyl)piperidin- 4-yl)-2-oxoimidazolidine-1- carboxamido)acetamido)-7-fluoro-2- hydroxy-3,4-dihydro-2H- benzo[e][1,2]oxaborinine-8-carboxylic acid | 824.89 |

### Example A1: Parenteral Composition

To prepare a parenteral pharmaceutical composition suitable for administration by injection, 100 mg of a compound disclosed herein, or a pharmaceutically acceptable salt, solvate, stereoisomer, tautomer, N-oxide, dimer, or trimer thereof, is dissolved in DMSO and then mixed with 10 ml of 0.9% sterile saline solution. The mixture is incorporated into a dosage unit suitable for administration by injection.

### Example A2: Oral Composition

To prepare a pharmaceutical composition for oral delivery, 400 mg of compound disclosed and the following ingredients are mixed intimately and pressed into single scored tablets.

Tablet Formulation

| Ingredient | Quantity per tablet (mg) |
|---|---|
| compound | 400 |
| cornstarch | 50 |
| croscarmellose sodium | 25 |
| lactose | 120 |
| magnesium stearate | 5 |

The following ingredients are mixed intimately and loaded into a hard-shell gelatin capsule.

Capsule Formulation

| Ingredient | Quantity per capsule (mg) |
|---|---|
| compound | 200 |
| lactose spray dried | 148 |
| magnesium stearate | 2 |

### BIOLOGICAL EXAMPLES

### Example I: Experimental Method for Penicillin-Binding Protein Binding Assays with Bocillin-FL Via Fluorescence Polarization

To determine the ability of boronic acid-based test PBP inhibitors to bind Penicillin Binding Proteins (PBPs), Bocillin-FL (fluorescently-labeled penicillin V; ThermoFisher Scientific) was used in a fluorescence polarization (FP) competition binding assay to assess inhibitor binding to PBP3 from Escherichia coli (K-12), PBP3 from Pseudomonas aeruginosa (PAO1), PBP1a and PBP3 from Acinetobacter baumannii (ATCC 19606), and PBP2 from Neisseria gonorrhoeae (FA19). PBPs were cloned and purified as described previously (E. coli PBP3, King, D. T, et al., ACS Infectious Diseases 2015, 1, 175-184; P. aeruginosa PBP3, Han et. al., PNAS 2010, 107 (51), 22002-22007; A. baumannii PBPs, Penwell et. al., Antimicrob. Agents Chemother. 2015, 59 (3), 1680-1689; N. gonorrhoeae PBP2, Singh et. al., J. Biol. Chem. 2019, 294 (38), 14020-14032). To establish assay conditions for competition binding, enzyme titration/saturation binding experiments were initially performed. Bocillin-FL was prepared at 0.2 μM in a buffer comprised of 50 mM Hepes (pH 8.0), 300 mM NaCl and 10% (v/v) glycerol for reactions with E. coli, P. aeruginosa and N. gonorrhoeae PBPs, and 25 mM Tris (pH 8.0), 200 mM NaCl, 10% (v/v) glycerol and 0.005% (v/v) Tween 20 for reactions with A. baumannii PBPs. Saturation binding was performed by mixing 40 μl of PBP solutions ranging in concentrations from 0-24 μM with 40 μl of the 0.2 μM Bocillin-FL solution, in individual wells of a black 384-well microplate. FP was measured immediately upon mixing (Excitation, 490 nm; Emission, 520 nm; g-factor, 0.96), using a Cytation3 (BioTek) microplate reader and measured continuously for up to 120 minutes. The FP response stabilized after 15 minutes for P. aeruginosa and A. baumannii PBP3, 30 minutes for E. coli PBP3 and A. baumannii PBP1a, and less than 1 minute for N. gonorrhoeae PBP2. In all instances, the FP signal showed a dose dependence on PBP concentration. The competition binding assay (80 μl final volume) was validated using beta-lactams and PBPs at final concentrations of: 1.5 μM for E. coli PBP3; 0.75 μM for P. aeruginosa PBP3; 1 μM for A. baumannii PBP1a; 0.2 μM for A. baumannii PBP3; and 0.25 μM for N. gonorrhoeae PBP2. Bocillin-FL was at 0.1 μM (0.05 μM with A. baumannii PBP1a) and beta-lactam concentrations ranged from 0-1,000 μM. E. coli PBP3 was incubated with increasing concentrations of ampicillin in a black 384-well microplate (Corning) for 30 minutes. P. aeruginosa PBP3 was incubated for 15 minutes with aztreonam, whereas A. baumannii PBP1a and PBP3 were incubated for 15 minutes with meropenem, and then Bocillin-FL was added followed immediately by the FP measurement for up to 60 minutes. For N. gonorrhoeae PBP2 assays, cefixime, ceftriaxone was mixed with Bocillin-FL, then enzyme was added, and the FP was immediately measured for up to 30 minutes. The beta-lactam potency was reported as the concentration of beta-lactam required to reduce the amount of PBP bound-Bocillin-FL by 50% (EC₅₀). The EC₅₀ of ampicillin for E. coli PBP3 was determined to be 1.4 μM. The EC₅₀ of aztreonam was determined to be <0.5 μM for P. aeruginosa PBP3. EC₅₀s for meropenem with A. baumannii PBP1a and PBP3 were determined to be <0.5 μM and 0.23 μM, respectively. EC₅₀ values for cefixime and ceftriaxone with N. gonorrhoeae PBP2 were 0.26 M and 0.27 μM, respectively. Binding assays for boronic acid PBP inhibitors were performed in an identical fashion for the respective PBPs.

Representative results for binding to E. coli PBP3 are shown in Table 5, where A represents a potency of >250 μM, B represents a potency between 50 μM and 250 μM inclusive, C represents a potency between 10 μM and 50 μM inclusive, and D represents potency <10 μM. NT=Not Tested.

TABLE 5
Binding affinity to E. coli PBP3 by Exemplary
Compounds in fluorescence polarization competition
binding assay using Bocillin-FL.

| | E. coli K12 | | E. coli K12 | | E. coli K12 |
|---|---|---|---|---|---|
| Ex. | PBP3 Potency | Ex. | PBP3 Potency | Ex. | PBP3 Potency |
| 1 | D | 2 | D | 3 | C |
| 4 | D | 5 | B | 6 | D |
| 7 | D | 8 | D | 9 | B |
| 10 | D | 11 | D | 12 | D |
| 13 | D | 14 | A | 15 | D |
| 16 | D | 17 | D | 18 | D |
| 19 | D | 20 | D | 21 | D |
| 22 | D | 23 | D | 24 | D |
| 25 | D | 26 | B | 27 | B |
| 28 | B | 29 | B | 30 | B |
| 31 | B | 32 | C | 33 | B |
| 34 | B | 35 | C | 36 | B |
| 37 | D | 38 | C | 39 | B |
| 40 | D | 41 | D | 42 | D |
| 43 | B | 44 | C | 45 | D |
| 46 | D | 47 | D | 48 | C |
| 49 | A | 50 | B | 51 | C |
| 52 | A | 53 | B | 54 | B |
| 56 | C | 57 | C | 58 | A |
| 59 | A | 60 | A | 61 | B |
| 64 | B | 65 | A | 66 | B |
| 120 | D | 160 | NT | 161 | NT |
| 162 | NT | 163 | NT | 164 | NT |
| 165 | NT | 166 | NT | 167 | NT |
| 168 | NT | 169 | NT | 170 | NT |
| 171 | A | 172 | A | 173 | B |
| 174 | B | 175 | D | 176 | B |
| 177 | C | 178 | D | 179 | B |
| 180 | D | 181 | B | 182 | D |
| 183 | B | 184 | D | 186 | A |
| 187 | C | 188 | C | 189 | C |
| 190 | C | 191 | D | 192 | C |
| 193 | D | 194 | C | 195 | D |
| 196 | A | 197 | C | 198 | C |
| 199 | NT | 200 | C | 201 | D |
| 202 | D | 203 | D | 204 | D |
| 205 | D | 206 | D | 207 | D |
| 208 | D | 209 | D | 210 | D |
| 211 | D | 212 | C | 213 | D |
| 214 | D | 215 | D | 216 | D |
| 217 | D | 218 | D | 219 | D |
| 220 | D | 221 | D | 222 | D |
| 223 | D | 224 | D | 225 | C |
| 226 | D | 227 | C | 228 | D |
| 229 | C | 230 | D | 231 | D |
| 232 | D | 233 | D | 234 | D |
| 235 | D | 236 | D | 237 | D |
| 238 | D | 239 | D | 240 | D |
| 241 | D | 242 | D | 243 | C |
| 244 | C | 245 | C | 246 | C |
| 247 | C | 248 | C | 249 | D |
| 250 | D | 251 | D | 252 | A |
| 253 | D | 254 | B | 255 | D |
| 256 | C | 257 | C | 258 | D |
| 259 | D | 260 | D | 261 | D |
| 262 | D | 263 | A | 264 | D |
| 265 | C | 266 | D | 267 | D |
| 268 | D | 269 | D | 270 | B |
| 271 | D | 272 | D | 273 | D |
| 274 | D | 275 | C | 276 | D |
| 277 | D | 278 | D | 279 | D |
| 280 | C | 281 | C | 282 | C |
| 283 | A | 284 | C | 285 | C |
| 286 | A | 287 | D | 288 | D |
| 289 | D | 290 | D | 291 | D |
| 292 | D | 293 | D | 294 | D |
| 295 | D | 296 | D | 297 | D |
| 298 | D | 299 | A | 300 | D |
| 301 | D | 302 | A | 303 | D |
| 304 | D | 305 | D | 306 | D |
| 307 | D | 308 | D | 309 | D |
| 310 | D | 400 | D | 401 | D |
| 402 | D | 403 | D | 404 | D |
| 405 | D | 406 | D | 407 | D |
| 408 | D | 409 | D | 410 | D |
| 411 | D | 412 | D | 413 | D |
| 414 | D | 415 | C | 416 | D |
| 417 | D | 418 | D | 419 | D |
| 420 | D | 421 | D | 422 | D |
| 423 | D | 424 | D | 425 | D |
| 426 | NT | 427 | D | 428 | D |
| 429 | D | 466 | D | 470 | B |
| 471 | D | 472 | B | 473 | D |
| 498 | B | 499 | C | | |

Representative results for binding to P. aeruginosa PBP3 are shown in Table 6, where A represents a potency of >250 μM, B represents a potency between 50 μM and 250 μM inclusive, C represents a potency between 10 μM and 50 μM inclusive, and D represents potency <10 μM. NT=Not Tested.

TABLE 6
Binding affinity to P. aeruginosa PBP3 by Exemplary
Compounds in fluorescence polarization competition
binding assay using Bocillin-FL.

| | P. aeruginosa | | P. aeruginosa | | P. aeruginosa |
|---|---|---|---|---|---|
| Ex. | PBP3 Potency | Ex. | PBP3 Potency | Ex. | PBP3 Potency |
| 1 | D | 2 | D | 3 | C |
| 4 | D | 5 | C | 6 | D |
| 7 | NT | 8 | D | 9 | C |
| 10 | D | 11 | D | 12 | D |
| 13 | D | 14 | A | 15 | D |
| 16 | D | 17 | D | 18 | D |
| 19 | D | 20 | D | 21 | D |
| 22 | D | 23 | D | 24 | D |
| 25 | D | 26 | B | 27 | C |
| 28 | C | 29 | C | 30 | B |
| 31 | C | 32 | D | 33 | C |
| 34 | B | 35 | D | 36 | B |
| 37 | D | 38 | D | 39 | C |
| 40 | D | 41 | D | 42 | D |
| 43 | B | 44 | D | 45 | D |
| 46 | D | 47 | D | 48 | C |
| 49 | A | 50 | D | 51 | D |
| 52 | B | 53 | C | 54 | B |
| 56 | D | 57 | D | 58 | A |
| 59 | A | 60 | B | 61 | C |
| 64 | B | 65 | B | 66 | C |
| 120 | D | 160 | D | 162 | C |
| 163 | D | 164 | D | 165 | C |
| 166 | D | 167 | B | 168 | D |
| 169 | A | 170 | C | 171 | A |
| 172 | B | 173 | C | 174 | C |
| 175 | D | 176 | D | 177 | D |
| 178 | D | 179 | D | 180 | D |
| 181 | B | 182 | D | 183 | D |
| 184 | D | | | 186 | C |
| 187 | D | 188 | D | 189 | D |
| 190 | D | 191 | D | 192 | D |
| 193 | D | 194 | D | 195 | D |
| 196 | B | 197 | D | 198 | C |
| 200 | D | 201 | D | 202 | D |
| 203 | D | 204 | D | 205 | D |
| 206 | D | 207 | NT | 208 | NT |
| 209 | NT | 210 | D | 211 | NT |
| 212 | NT | 213 | NT | 214 | D |
| 215 | D | 216 | D | 217 | D |
| 218 | D | 219 | D | 220 | D |
| 221 | D | 222 | D | 223 | D |
| 224 | D | 225 | D | 226 | D |
| 227 | D | 228 | D | 229 | D |
| 230 | D | 231 | D | 232 | D |
| 233 | D | 234 | D | 235 | D |
| 236 | D | 237 | D | 238 | D |
| 239 | D | 240 | D | 241 | D |
| 242 | D | 243 | D | 244 | D |
| 245 | D | 246 | D | 247 | D |
| 248 | C | 249 | D | 250 | D |
| 251 | D | 252 | A | 253 | D |
| 254 | B | 255 | D | 256 | D |
| 257 | D | 258 | D | 259 | D |
| 260 | D | 261 | D | 262 | D |
| 263 | C | 264 | D | 265 | D |
| 266 | D | 267 | D | 268 | D |
| 269 | D | 270 | D | 271 | D |
| 272 | D | 273 | D | 274 | D |
| 275 | D | 276 | D | 277 | D |
| 278 | D | 279 | D | 280 | D |
| 281 | D | 282 | D | 283 | B |
| 284 | D | 285 | D | 286 | A |
| 287 | D | 288 | D | 289 | D |
| 290 | D | 291 | D | 292 | D |
| 293 | D | 294 | D | 295 | D |
| 296 | D | 297 | D | 298 | D |
| 299 | C | 300 | D | 301 | D |
| 302 | B | 303 | D | 304 | D |
| 305 | D | 306 | D | 307 | D |
| 308 | D | 309 | D | 310 | D |
| 400 | D | 401 | D | 402 | D |
| 403 | D | 404 | D | 405 | D |
| 406 | D | 407 | D | 408 | D |
| 409 | D | 410 | D | 411 | D |
| 412 | D | 413 | D | 414 | D |
| 415 | D | 416 | D | 417 | D |
| 418 | D | 419 | D | 420 | D |
| 421 | D | 422 | D | 423 | D |
| 424 | D | 425 | D | 426 | D |
| 427 | D | 428 | D | 429 | D |
| 430 | D | 431 | D | 432 | D |
| 433 | D | 434 | D | 435 | D |
| 436 | D | 437 | D | 438 | D |
| 439 | D | 440 | D | 441 | D |
| 442 | D | 443 | D | 444 | D |
| 445 | D | 446 | D | 447 | D |
| 448 | D | 449 | D | 450 | D |
| 451 | D | 452 | D | 454 | D |
| 455 | D | 456 | D | 459 | D |
| 460 | D | 461 | D | 462 | C |
| 463 | D | 464 | D | 465 | D |
| 466 | D | 467 | D | 468 | D |
| 470 | C | 471 | D | 472 | C |
| 473 | D | 474 | D | 475 | D |
| 476 | D | 477 | D | 478 | D |
| 479 | D | 480 | D | 481 | D |
| 482 | D | 483 | D | 484 | D |
| 485 | D | 486 | D | 487 | D |
| 488 | D | 489 | D | 490 | D |
| 491 | D | 492 | D | 493 | D |
| 494 | D | 495 | D | 496 | D |
| 497 | D | 498 | C | 499 | D |
| 500 | D | 501 | D | 502 | D |
| 503 | D | 504 | NT | 505 | D |
| 506 | D | 509 | D | | |

Representative results for binding to A. baumannii PBP1b and PBP3 are shown in Table 7, where A represents a potency of >250 μM, B represents a potency between 50 μM and 250 μM inclusive, C represents a potency between 10 μM and 50 μM inclusive, and D represents potency <10 μM. NT=Not Tested.

TABLE 7
Binding affinity to A. baumannii PBP1a and PBP3
by Exemplary Compounds in the fluorescence polarization
competition binding assay using Bocillin-FL.

| | A. | A. | | A. | A. |
|---|---|---|---|---|---|
| | baumannii | baumannii | | baumannii | baumannii |
| | PBP1a | PBP3 | | PBP1a | PBP3 |
| Ex. | Potency | Potency | Ex. | Potency | Potency |
| 1 | NT | NT | 2 | NT | NT |
| 3 | NT | NT | 4 | NT | NT |
| 5 | NT | NT | 6 | NT | D |
| 7 | NT | NT | 8 | NT | D |
| 9 | NT | NT | 10 | NT | D |
| 11 | B | D | 12 | NT | NT |
| 13 | A | D | 14 | NT | A |
| 15 | NT | D | 16 | A | D |
| 17 | NT | D | 18 | NT | D |
| 19 | NT | D | 20 | A | D |
| 21 | NT | D | 22 | A | NT |
| 23 | A | D | 24 | A | D |
| 25 | NT | NT | 26 | NT | D |
| 27 | NT | NT | 28 | NT | NT |
| 29 | NT | NT | 30 | NT | NT |
| 31 | NT | NT | 32 | NT | NT |
| 33 | NT | NT | 34 | NT | NT |
| 35 | NT | NT | 36 | NT | NT |
| 37 | NT | NT | 38 | NT | NT |
| 39 | NT | NT | 40 | NT | NT |
| 41 | NT | NT | 42 | NT | NT |
| 43 | NT | NT | 44 | NT | NT |
| 45 | A | D | 46 | A | D |
| 47 | NT | NT | 48 | NT | B |
| 49 | NT | D | 120 | NT | D |
| 160 | NT | D | 161 | NT | D |
| 162 | NT | D | 163 | NT | D |
| 164 | A | D | 165 | NT | D |
| 166 | NT | D | 167 | NT | C |
| 168 | NT | D | 169 | NT | C |
| 170 | NT | D | 171 | NT | B |
| 200 | A | D | 201 | A | D |
| 202 | A | D | 203 | A | D |
| 204 | NT | D | 205 | A | D |
| 206 | A | D | 207 | A | D |
| 208 | NT | D | 209 | A | D |
| 210 | A | D | 211 | A | D |
| 212 | A | D | 213 | A | D |
| 214 | A | D | 215 | A | D |
| 216 | A | D | 217 | A | D |
| 218 | A | D | 219 | A | D |
| 220 | A | D | 221 | A | D |
| 222 | A | D | 223 | A | D |
| 224 | A | D | 225 | A | D |
| 226 | A | D | 227 | A | D |
| 228 | A | D | 229 | A | D |
| 230 | A | D | 231 | A | D |
| 232 | A | D | 233 | A | D |
| 234 | A | D | 235 | A | D |
| 236 | A | D | 237 | A | D |
| 238 | A | D | 239 | A | D |
| 240 | A | D | 241 | A | D |
| 242 | A | D | 243 | A | D |
| 244 | A | D | 245 | A | D |
| 246 | A | D | 247 | A | D |
| 248 | A | D | 249 | NT | D |
| 250 | A | D | 251 | A | D |
| 252 | A | B | 253 | A | D |
| 254 | A | C | 255 | A | D |
| 256 | A | D | 257 | A | D |
| 258 | A | D | 259 | A | D |
| 260 | A | D | 261 | A | D |
| 262 | A | D | 263 | A | C |
| 264 | A | D | 265 | A | D |
| 266 | A | D | 267 | A | D |
| 268 | A | D | 269 | A | D |
| 270 | A | D | 271 | A | D |
| 272 | A | D | 273 | A | D |
| 274 | A | D | 275 | A | D |
| 276 | A | D | 277 | A | D |
| 278 | A | D | 279 | A | D |
| 280 | A | D | 281 | A | D |
| 282 | A | D | 283 | A | B |
| 284 | A | D | 285 | A | D |
| 286 | A | B | 287 | A | D |
| 288 | A | D | 289 | A | D |
| 290 | A | D | 291 | A | D |
| 292 | A | D | 293 | A | D |
| 294 | A | D | 295 | A | D |
| 296 | A | D | 297 | A | D |
| 298 | A | D | 299 | A | C |
| 300 | A | D | 301 | A | D |
| 302 | A | B | 303 | A | D |
| 304 | A | D | 305 | A | D |
| 306 | A | D | 307 | A | D |
| 308 | NT | D | 309 | NT | D |
| 310 | NT | D | | | |
| 400 | NT | D | 401 | NT | D |
| 402 | NT | D | 403 | NT | D |
| 404 | NT | D | 405 | NT | D |
| 406 | NT | D | 407 | NT | D |
| 408 | NT | D | 409 | NT | D |
| 410 | NT | D | 411 | NT | D |
| 412 | NT | D | 413 | NT | D |
| 414 | NT | D | 415 | NT | D |
| 416 | NT | D | 417 | NT | C |
| 418 | NT | D | 419 | NT | D |
| 420 | NT | D | 421 | NT | D |
| 422 | NT | D | 423 | NT | D |
| 424 | NT | D | 425 | NT | D |
| 426 | NT | D | 427 | NT | D |
| 428 | NT | D | 429 | NT | D |
| 430 | NT | D | 431 | NT | D |
| 432 | NT | D | 433 | NT | D |
| 434 | NT | D | 435 | NT | C |
| 436 | NT | D | 437 | NT | D |
| 438 | NT | D | 439 | NT | D |
| 440 | NT | D | 441 | NT | D |
| 442 | NT | D | 443 | NT | D |
| 444 | NT | D | 445 | NT | D |
| 446 | NT | D | 447 | NT | D |
| 448 | A | D | 449 | A | D |
| 450 | A | D | 451 | NT | D |
| 452 | A | D | | | |
| 454 | A | D | 455 | NT | D |
| 456 | NT | D | 459 | NT | D |
| 460 | NT | D | 461 | NT | D |
| 462 | NT | D | 463 | NT | D |
| 464 | NT | D | 465 | NT | D |
| 466 | NT | D | 467 | NT | D |
| 468 | NT | D | 469 | NT | D |
| 470 | NT | C | 471 | NT | D |
| 472 | NT | D | 473 | NT | D |
| 474 | NT | D | 475 | A | D |
| 476 | NT | D | 477 | A | D |
| 478 | A | D | 479 | A | D |
| 480 | A | D | 481 | A | D |
| 482 | A | D | 483 | A | D |
| 484 | A | D | 485 | NT | D |
| 486 | NT | D | 487 | NT | D |
| 488 | NT | D | 489 | NT | D |
| 490 | NT | D | 491 | NT | D |
| 492 | NT | D | 493 | NT | D |

Representative results for binding to N. gonorrhoeae FA19 PBP2 are shown in Table 8, where A represents a potency of >250 μM, B represents a potency between 50 PM and 250 μM inclusive, and C represents a potency between 10 μM and 50 μM inclusive, and D represents potency <10 μM. NT=Not Tested.

TABLE 8
Binding affinity to N. gonorrhoeae FA19 PBP2
by Exemplary Compounds in fluorescence polarization
competition binding assay using Bocillin-FL.

| | N. gonorrhoeae | | N. gonorrhoeae | | N. gonorrhoeae |
|---|---|---|---|---|---|
| Ex. | PBP2 Potency | Ex. | PBP2 Potency | Ex. | PBP2 Potency |
| 1 | D | 2 | D | 3 | D |
| 4 | D | 5 | D | 6 | D |
| 7 | D | 8 | D | 9 | NT |
| 10 | D | 11 | D | 12 | D |
| 13 | D | 14 | B | 15 | D |
| 16 | D | 17 | D | 18 | D |
| 19 | D | 20 | D | 21 | D |
| 22 | D | 23 | D | 24 | D |
| 25 | D | 26 | C | 27 | C |
| 28 | D | 29 | D | 30 | D |
| 31 | D | 32 | D | 33 | D |
| 34 | B | 35 | D | 36 | C |
| 37 | D | 38 | D | 39 | C |
| 40 | D | 41 | D | 42 | D |
| 43 | B | 44 | D | 45 | D |
| 46 | D | 47 | D | 48 | D |
| 51 | D | 57 | D | 171 | C |
| 177 | D | 178 | D | 180 | D |
| 182 | D | 183 | D | 184 | D |
| 188 | D | 189 | D | 192 | D |
| 193 | D | 198 | D | | |

### Example II: Experimental Method for Penicillin-Binding Protein Binding Assays with Bocillin-FL Via Gel Filtration

Affinity to A. baumannii PBP1a and PBP2 was assessed a competitive equilibrium binding assay using Bocillin-FL as the reporter molecule. Enzyme was pre-incubated with increasing concentrations of inhibitors, prior to addition of Bocillin and further incubation for 15 minutes. PBP bound with Bocillin-FL was separated by gel filtration using 96-well Zeba Spin size exclusion plates, and the fluorescence measured. The inhibitor affinity (reported as the EC₅₀) was determined by plotting the fraction of PBP bound with Bocillin-FL at each inhibitor concentration against the inhibitor concentration, and fitting the data to the following equation:

$$y = {y}_{min} + [ {y}_{max} - {y}_{min} ] / [ 1 + {( \frac{x}{E {C}_{50}} )}^{n} ] \qquad Equation 1$$

where y is the fraction bound at a given inhibitor concentration, yₘᵢₙ is the fraction bound when the enzyme is completely inactivated, yₘₐₓ is the maximum (uninhibited) fraction bound, n is the Hill coefficient, and x is the inhibitor concentration.

Representative results for binding to A. baumannii PBP1a and PBP2 are shown in Table 9, where A represents a potency of >250 μM, B represents a potency between 50 μM and 250 μM inclusive, and C represents a potency between 10 μM and 50 μM inclusive, and D represents potency <10 μM. NT=Not Tested.

TABLE 9
Binding affinity to A. baumannii PBP1a and PBP2
by Exemplary Compounds in competition binding
assay using Bocillin-FL via gel filtration.

| | A. | A. | | A. | A. |
|---|---|---|---|---|---|
| | baumannii | baumannii | | baumannii | baumannii |
| | PBP1a | PBP2 | | PBP1a | PBP2 |
| Ex. | Potency | Potency | Ex. | Potency | Potency |
| 8 | C | C | 17 | NT | B |
| 23 | C | D | 24 | C | C |
| 45 | C | D | 46 | D | C |
| 49 | C | C | 120 | C | C |
| 160 | D | D | 163 | D | C |
| 164 | D | D | 166 | C | C |
| 167 | B | C | 168 | B | D |
| 169 | NT | B | 170 | B | B |
| 201 | C | NT | 202 | C | B |
| 203 | D | C | 204 | C | C |
| 205 | B | C | 207 | C | NT |
| 209 | C | NT | 214 | C | C |
| 216 | C | C | 219 | D | C |
| 220 | D | D | 222 | C | C |
| 223 | D | NT | 224 | D | NT |
| 225 | C | NT | 226 | C | NT |
| 227 | C | C | 228 | C | C |
| 230 | C | NT | 244 | C | NT |
| 243 | C | NT | 247 | NT | C |
| 249 | C | NT | 253 | C | D |
| 255 | C | C | 258 | C | D |
| 259 | C | D | 260 | NT | C |
| 261 | C | C | 266 | D | C |
| 268 | C | C | 269 | D | C |
| 271 | D | D | 272 | C | NT |
| 273 | NT | C | 274 | D | NT |
| 276 | D | NT | 277 | D | D |
| 278 | C | NT | 279 | D | C |
| 280 | C | C | 284 | D | D |
| 285 | C | C | 287 | C | D |
| 289 | D | C | 290 | NT | D |
| 293 | C | C | 295 | C | C |
| 296 | D | D | 297 | C | C |
| 298 | C | C | 300 | C | NT |
| 301 | C | NT | 303 | D | NT |
| 304 | D | NT | 305 | D | D |
| 306 | D | D | 307 | D | D |
| 308 | C | D | 309 | C | D |
| 310 | B | C | | | |
| 400 | C | C | 401 | D | C |
| 402 | D | C | 403 | C | C |
| 404 | D | D | 405 | C | C |
| 406 | D | C | 407 | D | C |
| 408 | D | D | 409 | D | D |
| 410 | D | D | 411 | D | D |
| 412 | D | D | 413 | D | C |
| 414 | D | D | 415 | D | C |
| 416 | D | D | 417 | C | D |
| 418 | D | D | 419 | D | D |
| 420 | C | C | 421 | D | D |
| 422 | D | D | 423 | D | D |
| 424 | D | D | 425 | C | C |
| 426 | C | C | 427 | D | D |
| 428 | D | D | 429 | D | D |
| 430 | D | D | 431 | D | D |
| 432 | D | D | 433 | D | D |
| 434 | D | D | 435 | D | D |
| 436 | D | D | 437 | D | D |
| 438 | D | D | 439 | C | D |
| 440 | D | D | 441 | D | D |
| 442 | D | D | 443 | D | D |
| 444 | D | D | 445 | D | D |
| 446 | C | D | 447 | D | D |
| 448 | D | D | 449 | D | D |
| 450 | D | D | 451 | D | D |
| 452 | C | D | 454 | D | D |
| 455 | D | D | 456 | D | D |
| 459 | D | D | 460 | C | C |
| 461 | C | C | 462 | D | NT |
| 463 | D | D | 466 | NT | C |
| 467 | D | NT | 468 | D | D |
| 471 | D | D | 473 | D | D |
| 474 | C | D | 475 | C | D |
| 476 | C | D | 477 | C | D |
| 478 | C | C | 479 | C | C |
| 480 | D | C | 481 | D | D |
| 482 | C | C | 483 | D | C |
| 484 | D | D | 485 | D | D |
| 486 | D | D | 487 | C | D |
| 488 | D | C | 489 | D | D |
| 490 | D | C | 491 | D | D |
| 492 | D | C | 493 | D | C |
| 494 | B | C | 495 | D | D |
| 496 | D | D | 497 | D | D |
| 500 | D | D | 501 | D | D |
| 502 | C | D | 503 | C | C |
| 504 | D | D | 505 | D | D |
| 600 | D | D | 601 | D | C |
| 602 | D | C | 603 | C | C |
| 604 | C | C | 605 | D | C |
| 606 | D | C | 607 | D | C |
| 608 | D | C | 609 | D | C |
| 610 | D | C | 611 | D | C |
| 612 | D | C | 613 | C | C |
| 614 | C | C | 615 | C | C |
| 616 | C | C | 617 | C | B |
| 618 | C | C | 619 | D | C |
| 620 | D | D | 621 | D | D |

### Example III: In Vitro Antibacterial Assays

To determine the ability of test compounds to inhibit the growth of bacterial strains, classic cell-based broth microdilution minimum inhibitory concentration (MIC) assays were employed. MIC assays are performed according to CLSI methods except where otherwise noted (CLSI, 2018 and CLSI, 2019). The reference type strain E. coli ATCC 25922; the wild-type parent strain E. coli AG100; the hyper-permeable E. coli 901C and E. coli D22; and the E. coli AG100A strain lacking the acrAB efflux pump encoding genes were used to determine the ability of the PBP inhibitors to penetrate the outer membrane of gram-negative bacteria and inhibit bacterial growth. Three additional challenge isolates of Klebsiella pneumoniae (K. pneumoniae 848844 producing SHV-11 and KPC-2, K. pneumoniae UMM producing SHV-5 and KPC-2 and K. pneumoniae SI-117 producing VIM-1) were used to further assess antibacterial activity in Enterobacterales and demonstrate activity of the PBP inhibitors irrespective of the beta-lactamase content of these organisms. The P. aeruginosa ATCC 27853 and A. baumannii ATCC 19606, along with the hyper-permeable P. aeruginosa ATCC 35151 and an engineered efflux pump-compromised strain of P. aeruginosa (ΔmexAB-oprM) were used to determine the ability of PBP inhibitors to penetrate the outer membrane of P. aeruginosa and A. baumannii and assess antibacterial activity against these important gram-negative organisms. Finally, a BSL-2 Burkholderia pseudomallei ΔpurM adenine auxotroph strain, Bp82, was used to assess potential utility against this bioweapon pathogen.

Briefly, cryo-preserved bacterial cultures of challenge strains are streaked for isolation on appropriate agar medium, in this case cation-adjusted Mueller Hinton agar (Enterobacterales, P. aeruginosa, and A. baumannii) or cation-adjusted Mueller Hinton agar supplemented with 0.6 mM adenine (B. pseudomallei Bp82). Following incubation to allow growth of the colonies, plates are sealed with parafilm and stored refrigerated for up to two weeks. For preparation of assay inoculum and to ensure low variability, at least 5 colonies are picked from the agar plates with an inoculating loop and aseptically transferred to a culture tube containing either 3 mL of cation-adjusted Mueller Hinton broth (CAMHB) for Enterobacterales, P. aeruginosa and A. baumannii, or 3 mL of cation-adjusted Mueller Hinton broth supplemented with 0.6 mM adenine for B. pseudomallei Bp82. The broth culture is grown for 3-5 hours at 37° C. with shaking at 200 rpm (Enterobacterales, P. aeruginosa and A. baumannii) or in a stationary ambient air incubator at 37° C. (B. pseudomallei Bp82). Meanwhile, 2-fold serial dilutions of test compounds are conducted in a 96-well plate with a final volume of 50 μL per well at 2-fold the final desired concentration. After the dilution plates are set up the growing cultures are then diluted in a cuvette containing CAMHB and the optical density is measured at 600 nm. The inoculum is diluted such that 50 μL of this culture in CAMHB (supplemented with 2×0.6 mM adenine for B. pseudomallei Bp82) results in a starting bacterial concentration of 2-8×10⁵ CFU/mL when added to the dilution plates. The plates are incubated for 16-20 hours for Enterobacterales and P. aeruginosa and 20-24 hours for A. baumannii and B. pseudomallei at 37° C. The MIC values are read visually as the lowest concentration well with no bacterial growth.

Representative results for MIC testing in Enterobacterales are shown in Table 10, where A represents an MIC≥128 μg/mL, B represents an MIC of 32 to 64 μg/mL, C represents an MIC from 8 to 16 μg/mL, D represents an MIC from 2 to 4 μg/mL, E represents an MIC≤1 μg/mL. NT=Not Tested.

TABLE 10
Inhibition of bacterial growth. Minimum inhibitory concentrations
of Exemplary Compounds for Enterobacterales in CAMHB.

| | | | | | | K. | K. | K. |
|---|---|---|---|---|---|---|---|---|
| | E. coli | E. coli | E. coli | E. coli | E. coli | pneumoniae | pneumoniae | pneumoniae |
| Ex. | 25922 | 901C | D22 | AG100 | AG100A | 848844 | UMM | SI-117 |
| 1 | A | E | D | B | C | A | B | A |
| 2 | A | E | D | B | B | A | B | A |
| 3 | B | D | C | B | C | A | B | A |
| 4 | A | E | C | A | C | A | A | A |
| 5 | A | E | C | B | B | A | A | A |
| 6 | B | E | D | C | C | A | B | A |
| 7 | B | E | D | C | C | A | B | A |
| 8 | D | E | E | NT | E | A | D | C |
| 9 | A | D | B | A | A | A | A | A |
| 10 | A | E | C | A | B | A | A | A |
| 11 | B | E | C | B | C | A | A | A |
| 12 | B | E | D | C | C | A | A | A |
| 13 | A | E | C | B | C | A | A | A |
| 14 | A | A | A | A | A | A | A | A |
| 15 | B | E | D | B | C | A | A | A |
| 16 | A | E | C | A | B | A | A | A |
| 17 | B | E | D | C | C | A | B | A |
| 18 | B | E | C | B | C | A | B | A |
| 19 | A | E | C | A | B | A | A | A |
| 20 | C | E | D | C | C | A | B | A |
| 21 | C | E | D | C | C | A | C | A |
| 22 | B | E | D | C | C | A | B | A |
| 23 | NT | NT | NT | NT | NT | NT | NT | NT |
| 24 | NT | NT | NT | NT | NT | NT | NT | NT |
| 25 | A | E | D | B | B | A | B | A |
| 26 | A | C | B | A | B | A | A | A |
| 27 | A | C | B | A | A | A | A | A |
| 28 | A | C | B | A | A | A | A | A |
| 29 | A | C | C | A | A | A | B | A |
| 30 | A | C | C | A | A | A | A | A |
| 31 | A | B | B | A | A | A | A | A |
| 32 | A | D | C | A | B | A | A | A |
| 33 | A | B | B | A | A | A | A | A |
| 34 | A | C | B | A | A | A | A | A |
| 35 | A | C | C | A | A | A | A | A |
| 36 | A | C | B | A | A | A | A | A |
| 37 | A | E | C | A | B | A | A | A |
| 38 | A | D | C | A | B | A | A | A |
| 39 | A | D | B | A | A | A | A | A |
| 40 | B | E | D | B | C | A | B | A |
| 41 | B | E | D | B | C | A | B | A |
| 42 | B | E | D | B | C | A | B | A |
| 43 | A | C | B | A | A | A | A | A |
| 44 | A | D | C | A | B | A | A | A |
| 47 | C | E | D | C | C | A | A | A |
| 48 | A | E | C | A | A | A | A | A |
| 50 | A | C | B | A | A | A | A | A |
| 51 | A | E | D | A | B | A | B | A |
| 52 | A | B | B | A | B | A | B | B |
| 53 | A | C | B | A | A | A | A | A |
| 54 | A | B | A | A | A | A | A | A |
| 56 | A | D | C | A | B | A | A | A |
| 57 | A | E | C | A | B | A | B | A |
| 58 | A | A | A | A | A | A | A | A |
| 59 | A | B | A | A | A | A | A | A |
| 60 | A | B | A | A | A | A | A | A |
| 61 | A | B | A | A | A | A | A | A |
| 64 | A | C | B | A | A | A | A | A |
| 65 | A | B | A | A | A | A | A | A |
| 66 | A | B | B | A | B | A | A | A |
| 160 | D | E | D | E | E | B | D | C |
| 171 | A | A | A | A | A | A | A | A |
| 172 | A | B | A | A | A | A | A | A |
| 173 | A | B | B | A | B | A | B | B |
| 174 | A | C | B | A | A | A | A | A |
| 175 | B | D | C | B | B | B | B | B |
| 176 | A | C | A | A | A | A | A | A |
| 177 | A | D | B | A | A | A | A | A |
| 178 | A | E | D | A | B | A | B | A |
| 179 | A | C | B | A | A | A | A | A |
| 180 | A | E | D | A | C | A | A | A |
| 181 | A | B | A | A | A | A | A | A |
| 182 | A | D | C | A | B | A | A | A |
| 183 | A | C | B | A | A | A | A | A |
| 184 | A | E | C | A | B | A | A | A |
| 186 | A | C | A | A | A | A | A | A |
| 187 | A | C | B | A | A | A | A | A |
| 188 | A | E | C | A | A | A | A | A |
| 189 | A | E | D | B | C | A | B | A |
| 190 | A | D | B | A | A | A | A | A |
| 191 | A | E | C | A | B | A | A | A |
| 192 | A | E | C | A | B | A | A | A |
| 193 | B | E | D | B | C | A | A | A |
| 194 | A | D | B | A | A | A | A | A |
| 195 | A | D | C | A | A | A | A | A |
| 196 | A | B | A | A | A | A | A | A |
| 197 | A | D | C | A | A | A | A | A |
| 198 | A | E | C | A | B | A | B | A |
| 498 | A | B | A | A | A | A | A | A |
| 499 | A | D | B | A | B | A | A | A |
| 504 | E | E | D | E | E | D | D | D |
| 505 | E | E | E | E | E | E | E | E |
| 506 | E | E | E | E | E | D | E | E |
| 509 | E | E | E | E | E | E | E | E |

Representative results for testing in P. aeruginosa and A. baumannii strains are shown in Table 12, where A represents an MIC≥128 μg/mL, B represents an MIC of 32 to 64 μg/mL, C represents an MIC from 8 to 16 μg/mL, D represents an MIC from 2 to 4 μg/mL, and E represents an MIC≤1 μg/mL. NT=Not Tested.

TABLE 12
Inhibition of bacterial growth. Minimum inhibitory
concentrations of Exemplary Compounds for P. aeruginosa
and A. baumannii strains in CAMHB.

| | P. aeruginosa | P. aeruginosa | P. aeruginosa | A. baumannii |
|---|---|---|---|---|
| Ex. | ATCC 27853 | ATCC 35151 | ΔmexAB-oprM | ATCC 19606 |
| 1 | A | E | B | B |
| 2 | A | E | C | A |
| 3 | A | E | B | B |
| 4 | A | C | B | A |
| 5 | A | C | B | A |
| 6 | A | E | C | C |
| 7 | B | E | C | C |
| 8 | E | E | E | E |
| 9 | A | C | A | A |
| 10 | B | E | B | B |
| 11 | A | C | B | A |
| 12 | A | C | B | B |
| 13 | A | D | B | B |
| 14 | A | A | A | A |
| 15 | A | D | B | B |
| 16 | A | D | A | A |
| 17 | B | E | C | D |
| 18 | A | D | B | B |
| 19 | A | D | B | A |
| 20 | A | E | C | B |
| 21 | C | E | B | D |
| 22 | A | E | C | B |
| 25 | A | D | B | A |
| 26 | A | C | A | A |
| 27 | A | C | A | A |
| 28 | A | C | A | A |
| 29 | B | D | C | B |
| 30 | A | C | A | A |
| 31 | A | D | A | A |
| 32 | A | D | B | A |
| 33 | A | C | A | A |
| 34 | A | C | A | A |
| 35 | A | D | A | A |
| 36 | A | C | A | A |
| 37 | A | E | C | A |
| 38 | A | D | B | A |
| 39 | A | D | A | A |
| 40 | B | E | C | B |
| 41 | A | E | C | B |
| 42 | A | E | C | B |
| 43 | A | C | A | A |
| 44 | A | D | B | A |
| 47 | A | E | C | B |
| 48 | A | C | A | A |
| 50 | A | D | B | A |
| 51 | A | D | B | A |
| 52 | A | C | A | A |
| 53 | A | C | A | A |
| 54 | A | B | A | A |
| 56 | A | E | A | A |
| 57 | A | D | A | A |
| 58 | A | A | A | A |
| 59 | A | B | A | A |
| 60 | A | B | A | A |
| 61 | A | B | A | A |
| 64 | A | C | A | A |
| 65 | A | C | A | A |
| 66 | A | C | B | A |
| 160 | E | E | E | E |
| 171 | A | A | A | A |
| 172 | A | B | A | A |
| 173 | A | C | B | A |
| 174 | A | D | B | A |
| 175 | B | E | C | A |
| 176 | B | D | B | A |
| 177 | A | D | A | A |
| 178 | B | E | C | B |
| 179 | A | D | A | A |
| 180 | A | D | B | A |
| 181 | A | C | A | A |
| 182 | A | E | C | A |
| 183 | A | C | A | A |
| 184 | A | D | A | A |
| 186 | A | D | A | A |
| 187 | A | D | B | A |
| 188 | A | C | B | A |
| 189 | A | E | C | A |
| 190 | A | D | B | A |
| 191 | A | E | C | A |
| 192 | A | C | B | A |
| 193 | A | D | B | A |
| 194 | A | D | B | A |
| 195 | B | E | C | A |
| 196 | A | C | A | A |
| 197 | A | E | B | A |
| 198 | A | C | A | B |
| 498 | A | C | A | A |
| 499 | A | D | B | A |
| 504 | E | E | E | E |
| 505 | E | E | E | E |
| 506 | E | E | E | E |
| 509 | E | E | E | E |

Representative results for testing in B. pseudomallei Bp82 are shown in Table 11, where A represents an MIC≥128 μg/mL, B represents an MIC of 32 to 64 μg/mL, C represents an MIC from 8 to 16 μg/mL, D represents an MIC from 2 to 4 μg/mL, and E represents an MIC≤1 μg/mL. NT=Not Tested.

TABLE 11
Inhibition of bacterial growth. Minimum inhibitory concentrations
of Exemplary Compounds for B. pseudomallei Bp82 in CAMHB.

| Ex. | B. pseudomallei Bp82 |
|---|---|
| 8 | E |
| 203 | E |
| 204 | E |
| 205 | E |
| 208 | E |
| 209 | E |
| 210 | E |
| 211 | E |
| 212 | E |
| 213 | E |
| 214 | D |
| 216 | E |
| 217 | C |
| 221 | E |
| 222 | E |
| 223 | E |
| 224 | E |
| 225 | E |
| 226 | E |
| 227 | E |
| 228 | E |
| 229 | E |
| 245 | E |
| 247 | E |
| 249 | E |
| 250 | E |
| 251 | E |
| 255 | E |
| 256 | E |
| 257 | E |
| 258 | E |
| 259 | E |
| 262 | E |
| 264 | E |
| 265 | E |
| 275 | E |
| 279 | E |
| 494 | E |
| 495 | E |
| 496 | E |
| 497 | E |
| 500 | E |
| 501 | D |
| 502 | E |
| 503 | E |
| 505 | E |
| 506 | E |
| 509 | E |
| 602 | E |
| 605 | E |
| 609 | E |
| 610 | E |
| 612 | E |
| 614 | E |
| 617 | E |
| 618 | E |

### Example III: In Vitro Antibacterial Assays in Iron-Depleted Cation-Adjusted Mueller-Hinton Broth

To determine the ability of test compounds to inhibit the growth of bacterial strains under conditions of iron-depletion, classic cell-based broth microdilution minimum inhibitory concentration (MIC) assays were employed. MIC assays are performed according to CLSI methods except where otherwise noted (CLSI, 2018 and CLSI, 2019). The reference type strain E. coli ATCC 25922 was used to determine the ability of the PBP inhibitors to inhibit the growth of Enterobacterales. Wild-type P. aeruginosa ATCC 27853, A. baumannii ATCC 17978 and A. baumannii ATCC 19606, along with the hyper-permeable P. aeruginosa ATCC 35151, an engineered efflux pump-compromised strain of P. aeruginosa (ΔmexAB-oprM) was used to determine the ability of PBP inhibitors to penetrate the outer membrane of P. aeruginosa and assess antibacterial activity against these important gram-negative organisms. Additionally, four challenge isolates of Pseudomonas aeruginosa (P. aeruginosa CDC-0054 producing VIM-4, OXA-50, and PDC; P. aeruginosa CDC-0090 producing KPC-5, OXA-50, and PDC; P. aeruginosa CDC-0095 producing OXA-50, and PDC), and five challenge isolates of Acinetobacter baumannii (A. baumannii 1258916 producing ADC-33, OXA-23, and OXA-82; A. baumannii CDC-0033 producing NDM-1, and OXA-94; A. baumannii CDC-0036 producing OXA-65, and OXA-24; A. baumannii CDC-0045 producing TEM-1D, OXA-23, and OXA-69; and A. baumannii CDC-0083 producing NDM-1, PER-7, OXA-23, and OXA-69) were used to further assess antibacterial activity in non-fermenters and demonstrate activity of the PBP inhibitors irrespective of the beta-lactamase content of these organisms. Additionally, two Burkholderia bioweapon pathogen surrogate strains, B. thailandensis ATCC 700388 and B. humptydooensis ATCC BAA-2767, were used to assess potential biodefense applications.

Briefly, cryo-preserved bacterial cultures of challenge strains are streaked for isolation on appropriate agar medium, in this case cation-adjusted Mueller Hinton agar. Following incubation to allow growth of the colonies, plates are sealed with parafilm and stored refrigerated for up to two weeks. For preparation of assay inoculum and to ensure low variability, at least 5 colonies are picked from the agar plates with an inoculating loop and aseptically transferred to a culture tube containing 3 mL of iron-depleted cation-adjusted Mueller Hinton broth (IDM)—see below for IDM preparation. The broth culture is grown for 3-5 hours at 37° C. with shaking at 200 rpm. Meanwhile, 2-fold serial dilutions of test compounds are conducted in a 96-well plate with a final volume of 50 μL per well at 2-fold the final desired concentration. After the dilution plates are set up the growing cultures are then diluted in a cuvette containing IDM and the optical density is measured at 600 nm. The inoculum is diluted such that 50 μL of this culture in IDM results in a starting bacterial concentration of 2-8×10⁵ CFU/mL when added to the dilution plates. The plates are incubated for 16-20 hours for Enterobacterales and P. aeruginosa and 20-24 hours for A. baumannii at 37° C. The MIC values are read visually as the lowest concentration well with no bacterial growth.

### Method for Iron-Depleted Cation-Adjusted Mueller Hinton Broth (IDM) Preparation:

Prepare cation-adjusted Mueller Hinton broth as per the manufacturer's recommendations and autoclave.
   Add 100 g/L Chelex 100 resin, cover with foil and incubate with stirring for 2 hours
   Remove Chelex 100 resin by filtration with 0.45 μm filter flask
   Add the following back to the medium:
   CaCl₂ dihydrate: 82.5 mg/L
   MgCl₂ hexahydrate: 94.1 mg/L
   ZnSO₄: 10 μM
   Adjust pH of medium to 7.3 with 5 N HCl
   Sterilize using a 0.22 μm filter flask

Representative results for testing compounds in iron-depleted media conditions are shown in Tables 13, 14 and 15, where A represents an MIC≥128 μg/mL, B represents an MIC of 32 to 64 μg/mL, C represents an MIC from 8 to 16 μg/mL, D represents an MIC from 2 to 4 μg/mL, and E represents an MIC≤1 μg/mL. NT=Not Tested.

TABLE 13
Inhibition of bacterial growth. Minimum inhibitory
concentrations of Exemplary Compounds for P. aeruginosa
strains in iron-depleted CAMHB (IDM).

| | ATCC | ATCC | ΔmexAB- | CDC- | CDC- | CDC- | CDC- |
|---|---|---|---|---|---|---|---|
| Ex. | 27853 | 35151 | oprM | 0054 | 0090 | 0095 | 0110 |
| 6 | B | D | C | A | A | A | B |
| 8 | E | E | E | E | D | E | E |
| 19 | A | D | B | A | A | A | A |
| 23 | E | E | E | E | C | E | E |
| 24 | D | E | E | E | C | E | E |
| 45 | E | E | E | E | E | E | E |
| 46 | D | E | E | D | D | E | E |
| 49 | D | E | E | E | D | E | E |
| 120 | E | E | E | E | E | E | E |
| 160 | E | E | E | E | E | E | E |
| 161 | D | E | D | E | A | E | E |
| 162 | D | E | D | D | D | C | D |
| 163 | E | E | E | E | D | E | E |
| 164 | E | E | E | E | E | E | E |
| 165 | D | E | D | E | D | E | E |
| 166 | E | E | E | E | D | E | E |
| 167 | A | B | A | A | A | A | A |
| 168 | C | D | C | B | B | C | B |
| 169 | A | A | A | A | A | A | A |
| 170 | A | D | A | A | A | A | A |
| 200 | E | E | E | E | E | E | E |
| 201 | E | E | E | E | E | E | E |
| 202 | D | E | E | E | D | D | E |
| 203 | E | E | E | E | E | D | E |
| 204 | E | E | E | E | E | E | E |
| 205 | E | E | E | E | E | E | E |
| 206 | D | E | E | E | E | E | E |
| 207 | D | E | E | D | E | D | E |
| 208 | E | E | E | D | E | E | E |
| 209 | E | E | E | D | E | E | E |
| 210 | D | E | E | E | E | E | E |
| 211 | D | E | E | D | E | D | E |
| 212 | D | E | E | C | E | D | E |
| 213 | D | E | E | D | D | E | E |
| 214 | D | E | E | D | E | D | E |
| 215 | D | E | E | C | E | D | E |
| 216 | D | E | E | C | E | C | E |
| 217 | D | E | E | E | D | D | E |
| 218 | D | E | E | C | E | C | D |
| 219 | D | E | E | C | E | C | E |
| 220 | D | E | E | C | E | C | E |
| 221 | E | E | E | D | E | C | E |
| 222 | E | E | E | D | D | C | E |
| 223 | D | E | E | D | D | D | E |
| 224 | D | E | E | D | E | D | E |
| 225 | D | E | E | D | E | D | E |
| 226 | D | E | E | C | E | D | E |
| 227 | E | E | E | E | D | E | E |
| 228 | E | E | E | E | D | D | E |
| 229 | E | E | E | D | D | E | D |
| 230 | E | E | E | E | D | E | E |
| 231 | E | E | E | D | D | D | E |
| 232 | D | E | E | D | D | D | D |
| 233 | E | E | E | E | E | D | E |
| 234 | D | E | E | D | D | B | E |
| 235 | D | E | E | C | D | D | E |
| 237 | D | E | E | C | D | D | E |
| 238 | D | E | E | D | D | D | E |
| 239 | D | E | E | D | D | D | E |
| 240 | E | E | E | E | D | D | E |
| 241 | E | E | E | E | D | D | E |
| 242 | D | E | E | D | E | D | D |
| 243 | E | E | E | D | E | C | E |
| 244 | D | E | E | D | E | B | D |
| 245 | D | E | E | D | E | D | E |
| 246 | D | E | E | D | D | D | E |
| 247 | D | E | E | D | D | D | E |
| 248 | D | E | E | D | C | C | E |
| 249 | D | E | E | D | E | B | E |
| 250 | D | E | E | C | D | B | D |
| 251 | E | E | E | C | D | C | E |
| 252 | A | D | A | A | A | A | A |
| 253 | D | E | E | C | D | D | D |
| 254 | A | E | A | A | A | A | A |
| 255 | E | E | E | E | E | D | E |
| 256 | D | E | E | D | D | D | D |
| 257 | D | E | E | D | D | D | D |
| 258 | D | E | E | E | E | E | E |
| 259 | E | E | E | D | E | D | E |
| 260 | D | E | E | D | D | D | D |
| 261 | D | E | E | D | D | D | D |
| 262 | E | E | E | D | E | D | E |
| 263 | B | D | C | C | B | A | A |
| 264 | E | E | E | E | E | D | E |
| 265 | D | E | E | D | D | D | E |
| 266 | D | E | E | D | D | C | D |
| 267 | D | E | E | D | D | D | D |
| 268 | C | E | E | C | D | B | C |
| 269 | D | E | E | D | D | C | C |
| 270 | B | E | E | A | B | A | A |
| 271 | D | E | E | D | D | A | C |
| 272 | D | E | E | C | D | A | D |
| 273 | D | E | E | D | E | B | E |
| 274 | D | E | E | D | D | A | D |
| 275 | D | E | E | C | C | C | E |
| 276 | D | E | E | C | E | C | D |
| 277 | D | E | E | D | D | C | C |
| 278 | D | E | E | D | D | C | D |
| 279 | D | E | E | E | D | D | E |
| 280 | D | E | E | D | D | C | D |
| 281 | D | E | E | C | D | D | D |
| 282 | D | E | E | D | D | C | E |
| 283 | A | D | A | A | A | A | A |
| 284 | D | E | E | E | E | E | E |
| 285 | C | E | E | C | D | A | C |
| 286 | A | C | A | A | A | A | A |
| 287 | D | E | E | D | D | D | E |
| 288 | D | E | E | D | D | C | D |
| 289 | D | E | E | D | D | D | D |
| 290 | D | E | E | D | D | D | E |
| 291 | D | E | E | D | D | A | D |
| 292 | E | E | E | E | E | A | A |
| 293 | D | E | E | E | D | D | E |
| 294 | E | E | E | E | E | E | E |
| 295 | D | E | E | D | E | E | E |
| 296 | E | E | E | E | E | E | E |
| 297 | E | E | E | D | E | E | E |
| 298 | D | E | E | E | E | E | E |
| 299 | C | E | D | D | D | D | C |
| 300 | D | E | E | E | E | D | E |
| 301 | E | E | E | E | E | E | E |
| 302 | B | D | D | B | B | A | A |
| 303 | E | E | E | E | E | E | E |
| 304 | D | E | E | D | E | E | E |
| 305 | D | E | E | D | E | D | E |
| 306 | D | E | E | D | E | E | E |
| 307 | D | E | E | D | E | E | E |
| 308 | E | E | E | E | D | D | E |
| 309 | D | E | E | E | D | D | E |
| 310 | E | E | E | E | D | D | E |
| 400 | D | E | E | E | D | D | E |
| 401 | D | E | E | E | E | E | E |
| 402 | D | E | E | E | E | E | E |
| 403 | D | E | E | E | D | D | E |
| 404 | D | E | E | E | D | D | E |
| 405 | D | E | E | D | D | D | D |
| 406 | E | E | E | E | E | E | E |
| 407 | E | E | E | E | E | D | E |
| 408 | E | E | E | E | E | E | E |
| 409 | E | E | E | E | E | D | E |
| 410 | E | E | E | E | E | D | E |
| 411 | E | E | E | E | E | E | E |
| 412 | E | E | NT | E | D | D | E |
| 413 | E | E | E | E | E | E | E |
| 414 | E | E | E | E | E | E | E |
| 415 | E | E | E | E | E | E | E |
| 416 | E | E | E | E | E | E | E |
| 417 | C | E | E | C | C | C | B |
| 418 | D | E | E | E | E | D | E |
| 419 | D | E | E | D | D | D | E |
| 420 | D | E | E | D | E | D | E |
| 421 | D | E | E | E | E | D | E |
| 422 | D | E | E | E | D | E | E |
| 423 | E | E | E | E | E | E | E |
| 424 | E | E | E | E | E | E | E |
| 425 | E | E | E | E | E | D | E |
| 426 | D | E | E | E | E | D | E |
| 427 | E | E | E | D | D | D | E |
| 428 | D | E | E | E | E | D | E |
| 429 | D | E | E | E | E | D | E |
| 430 | E | E | E | E | E | E | E |
| 431 | E | E | E | E | E | E | E |
| 432 | E | E | E | E | E | E | E |
| 433 | E | E | E | E | E | E | E |
| 434 | E | E | E | E | E | E | E |
| 435 | E | E | E | E | E | E | E |
| 436 | E | E | E | E | E | E | E |
| 437 | E | E | E | E | E | E | E |
| 438 | E | E | E | E | D | E | E |
| 439 | E | E | E | D | E | D | E |
| 440 | E | E | E | E | E | E | E |
| 441 | D | E | E | E | E | E | E |
| 442 | E | E | E | E | E | E | E |
| 443 | E | E | E | E | E | E | E |
| 444 | E | E | E | E | E | E | E |
| 445 | E | E | E | E | E | E | E |
| 446 | E | E | E | E | E | E | E |
| 447 | E | E | E | E | E | E | E |
| 448 | E | E | E | E | E | E | E |
| 449 | E | E | E | E | E | E | E |
| 450 | E | E | E | E | E | E | E |
| 451 | E | E | E | E | E | E | E |
| 452 | D | E | E | E | E | D | E |
| 454 | D | E | E | E | D | D | E |
| 455 | E | E | E | E | E | E | E |
| 456 | E | E | E | E | E | E | E |
| 459 | D | E | E | E | D | C | E |
| 460 | E | E | E | E | E | E | E |
| 461 | E | E | E | E | E | E | E |
| 462 | D | E | D | C | NT | B | C |
| 463 | E | E | E | E | E | E | E |
| 466 | D | E | E | E | D | D | E |
| 467 | E | E | E | D | E | E | E |
| 468 | E | E | E | E | E | E | E |
| 470 | D | E | E | E | D | E | C |
| 471 | E | E | E | E | E | E | E |
| 472 | D | E | E | E | D | E | E |
| 473 | D | E | E | E | D | E | E |
| 474 | D | E | E | E | E | D | E |
| 475 | D | E | E | E | E | E | E |
| 476 | E | E | E | E | E | E | E |
| 477 | E | E | E | E | E | E | E |
| 478 | D | E | E | D | D | D | D |
| 479 | D | E | E | E | D | E | E |
| 480 | D | E | E | E | D | E | D |
| 481 | D | E | E | E | D | E | E |
| 482 | D | E | E | E | D | E | D |
| 483 | D | E | E | D | D | D | D |
| 484 | D | E | E | D | D | D | D |
| 485 | D | E | E | E | E | E | E |
| 486 | D | E | E | E | D | E | E |
| 487 | E | E | E | E | D | E | E |
| 488 | D | E | E | E | E | D | E |
| 489 | D | E | E | E | D | E | D |
| 490 | D | E | E | C | D | D | D |
| 491 | D | E | E | E | D | D | E |
| 492 | E | E | E | E | E | D | E |
| 493 | D | E | E | D | E | E | E |
| 494 | E | E | E | E | E | C | E |
| 495 | E | E | E | E | E | B | E |
| 496 | E | E | E | E | E | C | E |
| 497 | D | E | E | E | C | B | E |
| 500 | E | E | E | E | D | E | E |
| 502 | E | E | E | E | E | D | E |
| 503 | E | E | E | E | E | E | E |
| 504 | E | E | E | E | E | E | E |
| 505 | E | E | E | E | E | E | E |
| 506 | E | E | E | E | E | E | E |
| 509 | E | E | E | E | E | E | E |
| 601 | E | E | E | E | D | D | E |
| 602 | E | E | E | E | E | D | E |
| 603 | E | E | E | E | D | D | E |
| 604 | E | E | E | E | E | D | E |
| 614 | E | E | E | D | E | D | E |
| 615 | E | E | E | E | E | E | E |
| 616 | E | E | E | E | E | E | E |
| 617 | E | E | E | E | E | E | E |
| 618 | E | E | E | E | E | D | E |
| 619 | E | E | E | E | E | E | E |
| 620 | D | E | E | D | D | A | A |
| 621 | E | E | E | E | E | E | E |
| 622 | D | E | E | E | E | D | E |
| 623 | E | E | E | E | E | E | E |

TABLE 14
Inhibition of bacterial growth. Minimum inhibitory
concentrations of Exemplary Compounds for A. baumannii
strains in iron-depleted CAMHB (IDM).

| | ATCC | ATCC | | CDC- | CDC- | CDC- | CDC- |
|---|---|---|---|---|---|---|---|
| Ex. | 17978 | 19606 | 1258916 | 0033 | 0036 | 0045 | 0083 |
| 6 | D | C | B | B | B | A | A |
| 8 | E | E | E | E | D | E | E |
| 19 | C | B | A | A | A | A | A |
| 23 | E | E | E | E | E | E | E |
| 24 | E | E | E | E | E | E | E |
| 45 | E | E | E | E | E | E | E |
| 45 | E | E | E | E | E | E | E |
| 49 | E | E | A | A | A | A | A |
| 120 | E | E | E | D | C | E | E |
| 160 | E | E | E | E | E | E | E |
| 161 | E | E | A | C | A | A | C |
| 162 | E | E | B | A | A | A | NT |
| 163 | E | E | E | E | E | E | NT |
| 164 | E | E | E | E | E | E | NT |
| 165 | E | E | D | B | A | E | NT |
| 166 | E | E | E | E | E | E | NT |
| 167 | B | A | A | A | A | A | NT |
| 168 | E | E | D | B | D | D | NT |
| 169 | A | A | A | A | A | A | NT |
| 170 | E | E | A | A | A | A | NT |
| 200 | E | E | E | D | D | E | E |
| 201 | E | E | E | D | D | E | E |
| 202 | E | E | E | C | D | E | E |
| 203 | E | E | E | D | E | E | E |
| 204 | E | E | E | D | D | E | E |
| 205 | E | E | E | D | E | E | E |
| 206 | E | E | E | D | E | E | E |
| 207 | E | D | D | C | C | D | D |
| 208 | E | E | E | D | D | E | E |
| 209 | E | E | E | D | D | E | E |
| 210 | E | E | E | C | D | E | D |
| 211 | E | E | E | D | D | E | E |
| 212 | E | E | D | C | C | D | B |
| 213 | E | E | E | C | D | D | C |
| 214 | E | E | D | C | C | D | A |
| 215 | E | E | D | C | C | C | A |
| 216 | E | E | D | C | C | D | A |
| 217 | E | D | D | C | C | E | A |
| 218 | E | D | D | C | C | C | C |
| 219 | E | E | D | C | C | C | C |
| 220 | E | E | D | D | C | D | C |
| 221 | E | E | D | D | D | E | B |
| 222 | E | E | D | D | D | E | B |
| 223 | E | E | D | C | C | E | D |
| 224 | E | E | D | C | C | E | D |
| 225 | E | E | E | D | D | E | E |
| 226 | E | E | E | D | D | E | E |
| 227 | E | E | E | D | D | E | E |
| 228 | E | E | E | D | D | E | E |
| 229 | E | E | D | C | D | E | E |
| 230 | E | E | D | D | D | E | E |
| 231 | E | E | D | C | D | D | A |
| 232 | D | D | D | B | C | C | A |
| 233 | E | E | E | C | D | D | E |
| 234 | E | E | E | C | D | E | E |
| 235 | E | E | D | C | D | D | E |
| 236 | E | E | D | C | C | C | A |
| 237 | E | E | D | C | C | C | A |
| 238 | E | E | D | C | C | NT | A |
| 239 | E | E | E | C | D | E | E |
| 240 | E | E | E | D | D | D | A |
| 241 | E | E | E | C | D | D | A |
| 242 | E | E | E | D | D | E | D |
| 243 | E | E | E | D | E | E | E |
| 244 | E | E | E | D | D | D | E |
| 245 | E | E | E | C | D | E | E |
| 246 | E | E | E | D | D | E | E |
| 247 | E | E | E | D | E | E | E |
| 248 | E | E | E | D | D | E | E |
| 249 | E | E | E | D | D | E | E |
| 250 | E | E | E | D | D | E | E |
| 251 | E | E | E | D | D | E | D |
| 252 | C | A | A | A | A | A | A |
| 253 | E | E | E | E | E | E | E |
| 254 | D | C | A | B | A | A | A |
| 255 | E | E | E | D | E | E | E |
| 256 | E | E | E | D | D | E | E |
| 257 | E | E | E | D | D | E | E |
| 258 | E | E | E | E | E | E | E |
| 259 | E | E | E | D | E | E | E |
| 260 | E | E | E | D | D | E | E |
| 261 | E | E | E | D | D | E | E |
| 262 | E | E | E | D | E | E | E |
| 263 | E | C | B | B | B | B | D |
| 264 | E | E | E | E | E | E | E |
| 265 | E | E | D | C | D | E | E |
| 266 | E | E | E | D | D | E | E |
| 267 | E | E | E | C | D | E | E |
| 268 | E | E | E | C | D | D | D |
| 269 | E | E | D | C | C | E | B |
| 270 | E | D | C | B | B | C | D |
| 271 | E | E | E | D | D | E | E |
| 272 | E | E | D | C | D | E | E |
| 273 | E | E | E | D | D | E | E |
| 274 | E | E | E | C | D | E | E |
| 275 | E | E | E | C | D | E | D |
| 276 | E | E | E | C | D | D | E |
| 277 | E | E | D | D | D | D | E |
| 278 | E | E | E | C | D | E | E |
| 279 | E | E | E | D | E | E | E |
| 280 | E | E | E | D | D | E | D |
| 281 | E | E | E | D | D | E | C |
| 282 | E | E | E | D | D | E | D |
| 283 | A | A | A | A | A | A | A |
| 284 | E | E | E | E | E | E | D |
| 285 | E | E | D | C | C | D | E |
| 286 | A | A | A | A | A | A | A |
| 287 | E | E | E | E | D | E | E |
| 288 | E | E | E | C | D | E | E |
| 289 | E | E | E | D | D | D | E |
| 290 | E | E | E | D | D | E | E |
| 291 | E | E | E | D | D | D | E |
| 292 | E | A | A | A | A | A | A |
| 293 | E | E | E | D | E | E | E |
| 294 | E | E | E | D | E | E | E |
| 295 | E | E | E | D | D | E | E |
| 296 | E | E | E | D | E | E | E |
| 297 | E | E | E | D | D | E | E |
| 298 | E | E | E | D | E | E | E |
| 299 | E | D | C | C | B | A | D |
| 300 | E | E | E | D | D | E | E |
| 301 | E | E | E | D | D | E | E |
| 302 | D | B | A | A | A | A | A |
| 303 | E | E | E | D | E | E | E |
| 304 | E | E | E | D | D | E | E |
| 305 | E | E | E | D | D | D | D |
| 306 | E | E | E | D | D | E | D |
| 307 | E | E | E | D | D | E | E |
| 308 | E | E | E | D | D | E | E |
| 309 | E | E | E | D | E | E | E |
| 310 | E | E | E | D | D | E | E |
| 400 | E | E | D | D | D | E | D |
| 401 | E | E | E | D | D | E | D |
| 402 | E | E | D | D | D | E | D |
| 403 | E | E | D | D | D | E | D |
| 404 | E | E | D | D | D | E | A |
| 405 | E | E | D | D | D | E | E |
| 406 | E | E | D | D | D | E | E |
| 407 | E | E | D | D | D | E | E |
| 408 | E | E | D | D | C | E | E |
| 409 | E | E | D | D | D | E | E |
| 410 | E | E | D | D | D | E | E |
| 411 | E | E | D | C | D | D | C |
| 412 | E | E | D | C | C | D | C |
| 413 | E | E | D | D | D | D | D |
| 414 | E | E | D | D | D | D | D |
| 415 | E | E | D | D | D | D | D |
| 416 | E | E | E | D | D | E | E |
| 417 | E | E | E | D | D | E | A |
| 418 | E | E | D | E | D | E | A |
| 419 | E | E | D | C | D | D | A |
| 420 | E | E | D | D | D | E | A |
| 421 | E | E | D | D | D | E | A |
| 422 | E | E | E | D | D | E | A |
| 423 | E | E | E | D | D | E | A |
| 424 | E | E | E | E | E | E | D |
| 425 | E | E | E | E | E | E | C |
| 426 | E | E | D | D | C | D | NT |
| 427 | E | E | E | D | E | E | NT |
| 428 | E | E | E | D | D | E | C |
| 429 | E | E | E | D | D | E | C |
| 430 | E | E | C | C | C | C | NT |
| 431 | E | E | E | E | E | E | NT |
| 432 | E | E | E | D | D | E | NT |
| 433 | E | E | E | E | E | E | NT |
| 434 | E | E | E | E | E | E | NT |
| 435 | E | E | E | E | E | E | NT |
| 436 | E | E | E | E | E | E | NT |
| 437 | E | E | E | E | E | E | NT |
| 438 | E | E | E | E | E | E | NT |
| 439 | E | E | E | E | E | E | NT |
| 440 | E | E | E | E | E | E | NT |
| 441 | E | E | E | E | E | E | NT |
| 442 | E | E | E | E | E | E | NT |
| 443 | E | E | E | E | E | E | NT |
| 444 | E | E | E | E | E | E | NT |
| 445 | E | E | E | D | E | E | NT |
| 446 | E | E | E | E | E | E | NT |
| 447 | E | E | E | E | E | E | NT |
| 448 | E | E | E | E | E | E | NT |
| 449 | E | E | E | E | E | E | NT |
| 450 | E | E | E | E | E | E | NT |
| 451 | E | E | E | E | E | E | NT |
| 452 | E | E | E | D | D | E | NT |
| 454 | E | E | E | D | E | E | NT |
| 455 | E | E | E | E | E | E | NT |
| 456 | E | E | E | E | E | E | NT |
| 459 | E | E | E | D | E | E | NT |
| 460 | E | E | E | E | E | E | NT |
| 461 | E | E | E | E | E | E | NT |
| 462 | E | C | B | B | B | B | NT |
| 463 | E | E | E | E | E | E | NT |
| 466 | E | E | D | D | D | D | D |
| 467 | E | E | E | E | E | E | NT |
| 468 | E | E | E | E | E | E | NT |
| 470 | E | C | A | C | B | A | A |
| 471 | E | E | E | D | D | E | A |
| 472 | E | D | C | C | C | C | A |
| 473 | E | E | D | D | D | E | A |
| 474 | E | E | E | E | E | E | NT |
| 475 | E | E | E | D | D | E | NT |
| 476 | E | E | E | E | E | E | NT |
| 477 | E | E | E | D | D | E | NT |
| 478 | E | E | E | C | D | E | NT |
| 479 | E | E | E | D | E | E | NT |
| 480 | E | E | E | D | D | E | NT |
| 481 | E | E | E | D | E | E | NT |
| 482 | E | E | E | D | E | E | NT |
| 483 | E | E | E | C | D | E | NT |
| 484 | E | E | E | D | D | E | NT |
| 485 | E | E | E | E | E | E | NT |
| 486 | E | E | E | D | E | E | NT |
| 487 | E | E | E | D | E | E | NT |
| 488 | E | E | E | D | D | E | NT |
| 489 | E | E | E | D | D | E | NT |
| 490 | E | E | D | D | D | E | NT |
| 491 | E | E | E | D | D | E | NT |
| 492 | E | E | E | E | D | E | NT |
| 493 | E | E | E | D | E | E | NT |
| 494 | E | E | E | E | E | E | NT |
| 495 | E | E | E | E | E | E | NT |
| 496 | E | E | E | E | E | E | NT |
| 497 | E | E | E | E | E | E | NT |
| 500 | E | E | E | E | E | E | NT |
| 501 | E | E | D | D | D | E | NT |
| 502 | E | E | E | E | E | E | NT |
| 503 | E | E | E | D | D | D | NT |
| 504 | E | E | E | E | E | E | NT |
| 505 | E | E | E | E | E | E | NT |
| 506 | E | E | E | E | E | E | NT |
| 509 | E | E | E | E | E | E | NT |

TABLE 15
Inhibition of bacterial growth. Minimum inhibitory concentrations of Exemplary
Compounds for Burkholderia strains in iron-depleted CAMHB (IDM).

| | B. thailandensis | B. humptydooensis | | B. thailandensis | B. humptydooensis |
|---|---|---|---|---|---|
| Ex. | ATCC 700388 | ATCC BAA-2767 | Ex. | ATCC 700388 | ATCC BAA-2767 |
| 6 | C | D | 308 | D | D |
| 8 | E | E | 309 | E | E |
| 19 | B | B | 310 | D | E |
| 23 | E | E | 400 | D | D |
| 24 | E | E | 401 | A | A |
| 45 | E | E | 402 | D | D |
| 46 | E | E | 403 | D | D |
| 49 | D | E | 404 | D | A |
| 120 | E | E | 405 | E | D |
| 160 | E | E | 406 | D | C |
| 161 | E | E | 407 | D | C |
| 162 | C | E | 408 | D | C |
| 163 | E | E | 409 | D | C |
| 164 | E | E | 410 | D | A |
| 165 | E | E | 411 | D | D |
| 166 | E | E | 412 | D | D |
| 167 | A | A | 413 | D | D |
| 168 | E | E | 414 | D | D |
| 169 | A | A | 415 | C | C |
| 170 | A | A | 416 | E | E |
| 200 | E | E | 417 | A | A |
| 201 | E | E | 418 | E | E |
| 202 | C | E | 419 | D | D |
| 203 | E | E | 420 | D | 3 |
| 204 | D | E | 421 | D | D |
| 205 | E | E | 422 | E | E |
| 206 | D | E | 423 | E | E |
| 207 | C | E | 424 | E | D |
| 208 | E | E | 425 | E | E |
| 209 | E | E | 426 | E | E |
| 210 | E | E | 427 | E | E |
| 211 | D | E | 428 | A | A |
| 212 | D | E | 429 | C | A |
| 213 | D | E | 430 | E | E |
| 214 | C | E | 431 | E | E |
| 215 | C | E | 432 | E | E |
| 216 | D | E | 432 | E | E |
| 217 | D | E | 433 | E | E |
| 218 | D | D | 434 | E | E |
| 219 | C | C | 435 | E | E |
| 220 | C | B | 436 | E | E |
| 221 | D | E | 437 | E | E |
| 222 | D | D | 438 | E | E |
| 223 | D | E | 439 | E | E |
| 224 | D | E | 440 | E | E |
| 225 | E | E | 441 | E | E |
| 226 | E | E | 442 | E | E |
| 227 | E | E | 443 | E | E |
| 228 | E | E | 444 | E | E |
| 229 | E | e | 445 | E | E |
| 230 | D | D | 446 | E | E |
| 231 | D | C | 447 | E | E |
| 232 | E | A | 448 | E | E |
| 233 | D | E | 449 | E | D |
| 234 | C | E | 450 | E | C |
| 235 | C | E | 451 | E | E |
| 236 | C | D | 542 | E | 3 |
| 237 | C | D | 454 | E | E |
| 238 | C | A | 455 | E | E |
| 239 | C | B | 456 | E | E |
| 240 | D | D | 459 | E | E |
| 241 | D | D | 460 | E | E |
| 242 | D | C | 461 | E | E |
| 243 | D | D | 462 | A | A |
| 244 | A | D | 463 | E | E |
| 245 | D | E | 466 | D | C |
| 246 | D | D | 467 | E | D |
| 247 | E | E | 468 | E | E |
| 248 | D | E | 470 | A | C |
| 249 | D | E | 471 | E | E |
| 250 | A | E | 472 | D | D |
| 251 | E | E | 473 | E | E |
| 252 | A | A | 474 | D | E |
| 253 | D | NT | 475 | E | D |
| 254 | A | A | 476 | E | E |
| 255 | E | E | 477 | E | E |
| 256 | E | E | 478 | D | C |
| 257 | E | D | 479 | E | D |
| 258 | E | E | 480 | D | C |
| 259 | E | E | 481 | D | B |
| 260 | A | NT | 482 | E | D |
| 261 | D | D | 483 | B | A |
| 262 | E | E | 484 | B | A |
| 263 | C | C | 485 | D | D |
| 264 | E | D | 486 | D | D |
| 265 | E | E | 487 | E | D |
| 266 | D | C | 488 | C | A |
| 267 | D | D | 489 | D | B |
| 268 | A | A | 490 | B | A |
| 269 | A | A | 491 | D | C |
| 270 | A | A | 492 | E | E |
| 271 | C | D | 493 | E | E |
| 272 | D | D | 494 | E | E |
| 273 | D | D | 495 | E | E |
| 274 | A | D | 496 | E | E |
| 275 | E | E | 497 | E | E |
| 276 | A | B | 500 | E | E |
| 277 | B | A | 501 | E | E |
| 278 | D | D | 502 | E | E |
| 279 | E | E | 503 | E | E |
| 280 | D | D | 504 | E | E |
| 281 | D | E | 505 | E | E |
| 282 | E | E | 506 | E | E |
| 283 | A | A | 509 | E | E |
| 284 | E | E | 600 | A | A |
| 285 | D | D | 601 | E | E |
| 286 | A | A | 602 | E | E |
| 287 | D | E | 603 | E | E |
| 288 | D | D | 604 | E | E |
| 289 | C | D | 605 | E | E |
| 290 | D | E | 606 | E | E |
| 291 | D | E | 607 | E | E |
| 292 | A | A | 608 | E | E |
| 293 | E | E | 609 | E | E |
| 294 | E | E | 610 | E | E |
| 295 | D | E | 611 | E | E |
| 296 | D | D | 612 | E | E |
| 297 | D | E | 613 | E | E |
| 298 | E | E | 614 | E | E |
| 299 | A | A | 615 | E | E |
| 300 | D | E | 616 | D | E |
| 301 | D | D | 617 | E | E |
| 302 | A | A | 618 | E | E |
| 303 | E | E | 619 | E | E |
| 304 | D | D | 620 | A | A |
| 305 | D | D | 621 | D | E |
| 306 | D | D | 622 | D | NT |
| 307 | C | C | 623 | E | NT |

### Example IV: In Vitro Antibacterial Assays for N. gonorrhoeae Strains

Additional antibacterial testing of the series was performed in 8 reference strains of N. gonorrhoeae (ATCC 49226, FA1090, MS 11, WHO G, WHO L, WHO M, WHO K, WHO X, WHO Z, WHO Q, CDC-0197). ATCC 49226, FA1090, MS11, WHO G, WHO L, and WHO M produce wild type or wild-type like PBP2. WHO K, WHO X, WHO Z, WHO Q, and CDC-0197 produce mosaic PBP2. Liquid broth-based assays were used for antibacterial testing of PBP inhibitors in Neisseria gonorrhoeae. Briefly, cryo-preserved bacterial cultures of clinical strains were streaked for isolation on Chocolate Agar (72 g/L (2×) GC Agar Base (BD #228950) and 2% (2×) Hemoglobin was autoclaved at 121° C. for 20 minutes to sterilize. Once cooled to ˜50° C. the 2×GC Agar Base and 2× Hemoglobin solutions are combined and 1% IsoVitaleX Enrichment (BD #211876) was added to the solution). Strains were incubated at 36° C. and 5% CO₂ to allow growth of colonies, 24 hours before inoculum preparation. 2-fold serial dilutions of test compounds were conducted in a 96 well plate with a final volume of 75 μL per well at 2-fold the final desired concentration in Fastidious broth (Remel #R07664). For preparation of assay inoculum, a direct suspension was prepared by aseptically swabbing 10-15 colonies from agar plates into culture tubes containing 2 mL of fresh sterile saline. After the dilution plates were set up, direct suspensions were then diluted in a cuvette containing sterile saline and the optical density was measured at 600 nm. Inocula were diluted such that 75 μL of this culture in Fastidious broth results in a starting bacterial concentration of 5×10⁵ CFU/mL when added to the dilution plates. The plates were incubated for ˜24 hours at 36° C. and 5% CO₂. The MIC was read visually as the lowest concentration well that completely inhibits bacterial growth.

Representative results for testing in N. gonorrhoeae strains are shown in Table 16, where A represents an MIC≥64 μg/mL, B represents an MIC of 16 to 32 μg/mL, C represents an MIC from 4 to 8 μg/mL, D represents an MIC from 1 to 2 μg/mL, and E represents an MIC≤1 μg/mL. NT=Not Tested.

TABLE 16
Inhibition of bacterial growth. Minimum Inhibitory Concentrations
of Exemplary Compounds for N. gonorrhoeae strains.

| | ATCC | FA | | WHO | WHO | WHO | WHO | WHO | WHO | WHO | CDC- |
|---|---|---|---|---|---|---|---|---|---|---|---|
| Ex. | 49226 | 1090 | MS11 | G | K | L | M | X | Z | Q | 0197 |
| 1 | E | E | E | E | E | D | E | D | E | E | E |
| 2 | C | E | C | D | B | A | C | B | C | C | C |
| 3 | E | E | D | E | C | C | E | C | D | D | D |
| 4 | E | E | E | E | D | C | E | C | D | D | D |
| 5 | E | E | E | E | D | C | E | C | E | E | E |
| 6 | E | E | E | E | E | E | E | E | E | E | E |
| 7 | E | E | E | E | E | E | E | E | E | E | E |
| 8 | E | E | E | E | D | C | E | C | E | E | D |
| 9 | D | E | D | E | D | C | E | D | E | D | E |
| 10 | E | E | E | E | E | E | E | E | E | E | E |
| 11 | E | E | E | E | E | D | E | D | E | E | E |
| 12 | E | E | E | E | E | E | E | E | E | E | E |
| 13 | E | E | E | E | E | E | E | E | E | E | E |
| 14 | A | A | A | A | A | A | A | A | A | A | A |
| 15 | E | E | E | E | E | E | E | E | E | E | E |
| 16 | E | E | E | E | E | E | E | E | E | E | E |
| 17 | E | E | E | E | E | E | E | E | E | E | E |
| 18 | E | E | E | E | E | E | E | E | E | E | E |
| 19 | E | E | E | E | E | E | E | E | E | E | E |
| 20 | E | E | E | E | D | D | E | D | E | E | E |
| 21 | E | E | E | E | E | E | E | E | E | E | E |
| 22 | D | E | D | E | C | B | E | C | D | D | D |
| 23 | E | E | E | E | E | C | E | D | E | E | E |
| 24 | E | E | E | E | C | C | E | B | D | D | D |
| 25 | E | E | E | E | D | D | E | C | E | D | E |
| 26 | B | B | B | B | B | B | C | B | B | B | B |
| 27 | B | C | B | C | B | B | C | B | B | B | B |
| 28 | B | C | B | C | B | B | C | B | B | B | B |
| 29 | B | C | B | B | B | B | C | B | B | B | B |
| 30 | B | C | B | C | B | B | C | B | B | B | B |
| 31 | B | C | B | B | B | B | C | B | B | B | B |
| 32 | D | E | D | E | C | B | E | B | D | D | D |
| 33 | B | C | B | B | B | B | C | B | B | B | B |
| 34 | B | C | B | C | B | B | C | B | B | B | B |
| 35 | D | E | D | D | C | B | E | B | C | C | C |
| 36 | B | D | B | C | B | B | D | B | B | B | B |
| 37 | E | E | D | E | C | B | E | B | D | D | D |
| 38 | D | E | D | E | C | B | E | B | D | C | D |
| 39 | D | E | D | E | B | B | E | B | D | C | D |
| 40 | E | E | E | E | D | D | E | D | E | E | E |
| 41 | E | E | E | E | E | D | E | D | E | E | E |
| 42 | E | E | E | E | D | D | E | D | E | E | E |
| 43 | C | D | C | C | B | B | D | B | C | C | D |
| 44 | D | E | D | D | C | B | E | B | D | D | D |
| 45 | E | E | E | E | D | C | E | C | D | D | D |
| 46 | E | E | E | E | D | C | E | D | E | E | E |
| 47 | E | E | E | E | C | C | E | C | D | D | D |
| 48 | E | E | D | E | C | C | E | B | D | D | D |
| 50 | C | D | C | D | B | B | D | B | B | B | B |
| 51 | E | E | E | E | D | C | E | C | D | D | E |
| 52 | B | B | B | B | B | C | B | B | B | B | B |
| 53 | D | D | D | D | B | B | D | B | B | B | C |
| 54 | B | C | B | B | B | B | C | B | B | B | C |
| 56 | D | E | D | D | B | B | E | B | C | C | C |
| 57 | E | E | E | E | C | B | E | C | D | D | E |
| 58 | B | B | B | B | B | B | B | B | B | B | B |
| 59 | B | B | B | B | B | B | C | B | B | B | B |
| 60 | B | C | B | B | B | B | C | B | B | B | B |
| 61 | B | B | B | B | B | B | B | B | B | B | B |
| 64 | C | C | C | C | B | B | D | B | C | B | C |
| 65 | B | C | B | B | B | B | C | B | B | B | B |
| 66 | B | C | B | C | B | B | C | B | B | B | B |
| 171 | A | A | A | A | A | A | A | A | A | A | A |
| 172 | B | C | B | B | B | B | C | B | B | B | B |
| 173 | B | C | B | C | B | B | C | B | B | B | B |
| 174 | C | D | C | D | B | B | D | B | B | B | B |
| 175 | D | E | D | D | B | B | E | B | C | C | C |
| 176 | C | D | C | C | B | B | D | B | B | B | B |
| 177 | D | E | D | E | C | C | E | B | C | C | D |
| 178 | E | E | E | E | D | D | E | C | E | D | E |
| 179 | B | D | B | B | B | B | C | B | B | B | B |
| 180 | E | E | D | E | D | C | E | C | E | D | E |
| 181 | B | D | C | C | B | B | B | B | B | B | B |
| 182 | D | E | D | E | C | B | E | B | D | D | D |
| 183 | C | D | C | C | B | B | D | B | B | B | B |
| 184 | E | E | E | E | D | C | E | C | E | D | E |
| 188 | E | E | E | E | D | C | E | C | D | D | D |
| 189 | E | E | E | E | D | C | E | C | D | D | D |
| 190 | C | E | C | D | B | B | D | B | C | C | C |
| 191 | D | E | D | E | C | C | E | B | D | D | D |
| 192 | D | E | D | D | C | C | E | B | D | D | D |
| 193 | E | E | E | E | D | C | E | C | E | D | E |
| 194 | C | D | C | C | B | B | C | B | B | B | B |
| 195 | C | E | C | D | B | B | D | B | C | C | C |
| 196 | B | C | B | B | B | B | B | B | B | B | B |
| 197 | B | D | B | C | B | B | C | B | B | B | B |
| 198 | E | E | E | E | C | D | E | C | D | D | E |
| 200 | C | D | B | C | B | A | C | A | B | B | A |
| 201 | B | D | B | C | B | A | C | A | A | B | A |
| 202 | B | C | B | B | A | A | B | A | A | A | A |
| 203 | C | E | C | C | A | B | C | A | B | B | B |
| 204 | B | D | B | C | A | A | C | A | B | A | B |
| 205 | B | D | C | C | A | A | C | A | B | B | B |
| 206 | B | D | B | C | A | A | C | A | B | B | B |
| 207 | B | C | B | B | A | A | B | A | B | B | B |
| 208 | C | D | C | C | B | A | C | A | B | B | B |
| 209 | C | D | C | C | A | A | C | A | B | B | B |
| 210 | B | E | B | C | A | A | B | A | B | A | A |
| 211 | B | D | B | B | A | A | B | A | B | A | B |
| 212 | B | C | B | B | A | A | B | A | A | A | B |
| 213 | B | D | B | B | A | A | B | A | A | A | A |
| 214 | C | D | B | C | A | A | C | A | B | A | B |
| 215 | B | D | B | C | A | A | C | A | B | A | B |
| 216 | B | C | B | C | A | A | C | A | B | B | B |
| 217 | B | D | B | B | A | A | B | A | B | A | B |
| 218 | B | D | B | B | A | A | B | A | A | A | B |
| 219 | B | D | B | B | A | A | C | A | B | A | A |
| 220 | B | D | B | C | A | A | C | A | A | A | A |
| 221 | B | D | B | C | A | A | C | A | A | A | A |
| 222 | B | D | B | C | A | A | C | A | A | A | A |
| 223 | B | C | B | B | A | A | B | A | B | A | A |
| 224 | B | C | A | B | A | A | B | A | A | A | A |
| 225 | B | D | B | B | A | A | B | A | B | A | B |
| 226 | B | D | B | B | A | A | B | A | B | A | A |
| 227 | B | D | B | C | A | A | C | A | B | A | A |
| 228 | A | D | A | B | A | A | B | A | A | A | A |
| 229 | B | C | B | C | B | A | C | A | B | B | B |
| 230 | C | D | C | C | B | B | C | A | C | B | B |
| 231 | B | D | B | C | B | A | C | A | B | B | B |
| 232 | B | D | B | B | A | A | C | A | B | A | A |
| 233 | C | D | B | C | B | A | C | A | B | B | B |
| 234 | C | D | B | C | B | A | C | A | B | B | B |
| 235 | B | D | B | C | B | A | C | A | B | B | B |
| 237 | B | D | B | C | A | A | C | A | B | A | B |
| 238 | B | D | B | C | A | A | C | A | B | A | B |
| 239 | B | D | B | C | A | A | C | A | B | A | B |
| 240 | B | D | B | C | A | A | C | A | B | B | B |
| 241 | C | D | B | C | B | A | C | A | B | B | B |
| 242 | B | D | B | B | A | A | B | A | B | A | A |
| 243 | B | C | B | B | A | A | C | A | B | A | B |
| 244 | A | C | A | B | A | A | B | A | A | A | A |
| 245 | B | C | B | B | A | A | B | A | A | A | A |
| 246 | B | C | B | B | A | A | B | A | B | A | A |
| 247 | B | C | B | B | A | A | B | A | B | B | A |
| 248 | B | C | B | B | B | A | C | A | B | B | B |
| 249 | B | D | B | B | A | A | B | A | B | A | A |
| 250 | A | C | B | A | A | A | B | A | A | A | A |
| 251 | B | C | B | B | A | A | B | A | B | A | A |
| 252 | A | A | A | A | A | A | A | A | A | A | A |
| 253 | B | D | B | B | A | A | C | A | B | B | A |
| 254 | A | A | A | A | A | A | A | A | A | A | A |
| 255 | B | D | B | C | A | A | C | A | B | B | B |
| 256 | B | C | A | B | A | A | B | A | B | A | A |
| 257 | A | C | B | A | A | A | B | A | B | A | A |
| 258 | C | D | B | C | A | A | C | A | C | B | B |
| 259 | B | D | B | B | A | A | C | A | B | B | B |
| 260 | B | C | B | B | A | A | B | A | B | B | B |
| 261 | B | C | B | B | A | A | C | A | C | B | B |
| 262 | B | C | B | B | A | A | B | A | B | B | B |
| 263 | A | A | A | A | A | A | A | A | A | A | A |
| 264 | B | D | B | B | B | A | C | A | C | B | B |
| 265 | B | C | B | B | A | A | C | A | C | B | B |
| 266 | B | D | B | B | B | A | C | A | C | B | B |
| 267 | B | D | B | B | A | A | C | A | C | B | B |
| 268 | B | C | B | B | A | A | B | A | B | B | B |
| 269 | B | C | B | B | A | A | C | A | B | B | B |
| 270 | A | B | A | A | A | A | A | A | A | A | A |
| 271 | B | D | B | B | A | A | C | A | C | B | B |
| 272 | B | C | B | B | A | A | B | A | C | B | B |
| 273 | B | C | B | B | A | A | B | A | C | B | B |
| 274 | B | C | B | B | A | A | B | A | B | B | B |
| 275 | B | C | B | B | A | A | B | A | B | B | B |
| 276 | B | D | B | B | A | A | C | A | C | B | B |
| 277 | B | D | B | B | A | A | B | A | B | B | B |
| 278 | B | C | B | B | A | A | B | A | B | B | B |
| 279 | C | E | C | C | B | B | D | A | C | B | C |
| 280 | B | C | B | B | A | A | B | A | B | A | A |
| 281 | A | C | A | B | A | A | B | A | B | A | A |
| 282 | B | C | B | C | B | A | C | A | C | B | B |
| 283 | A | A | A | A | A | A | A | A | A | A | A |
| 284 | C | D | C | C | B | B | C | A | C | C | B |
| 285 | A | C | A | A | A | A | A | A | A | A | A |
| 286 | A | A | A | A | A | A | A | A | A | A | A |
| 287 | C | D | C | C | B | A | C | A | C | C | B |
| 288 | B | D | C | C | B | A | C | A | C | B | B |
| 289 | B | D | C | C | B | A | C | A | C | C | B |
| 290 | B | D | B | B | A | A | B | A | C | B | B |
| 291 | B | C | B | B | A | A | B | A | C | B | B |
| 292 | A | B | A | A | A | A | A | A | A | A | A |
| 293 | B | D | B | B | A | A | B | A | A | A | A |
| 294 | B | D | B | B | A | A | B | A | B | A | A |
| 295 | B | D | B | B | A | A | B | A | A | A | A |
| 296 | B | D | B | B | A | A | B | A | A | A | A |
| 297 | B | D | B | B | A | A | B | A | A | A | A |
| 298 | B | D | B | B | A | A | B | A | A | A | A |
| 299 | A | B | A | A | A | A | A | A | A | A | A |
| 300 | B | C | B | B | A | A | B | A | A | A | A |
| 301 | B | C | B | B | A | A | B | A | A | A | A |
| 302 | A | A | A | A | A | A | A | A | A | A | A |
| 303 | C | D | C | C | A | A | C | A | B | A | B |
| 304 | B | D | B | B | A | A | B | A | A | A | A |
| 305 | B | D | B | B | A | A | B | A | A | A | A |
| 306 | B | D | B | B | A | A | B | A | A | A | A |
| 307 | B | D | B | B | A | A | B | A | A | A | A |
| 498 | A | C | NT | NT | NT | NT | NT | NT | A | NT | NT |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound of Formula (Ia) or (Ib), or a pharmaceutically acceptable salt, solvate, stereoisomer, tautomer, N-oxide, dimer, or trimer thereof: 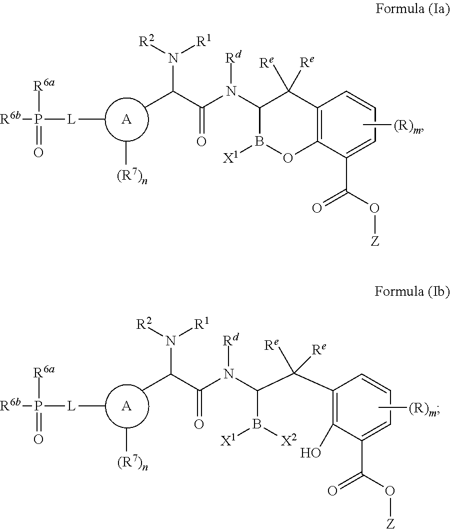
wherein:
R¹ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
R² is hydrogen, C₁-C₆ alkyl, —C(═O)R³, —S(═O)₂R³, —C(═O)N(R⁴)₂, or —S(═O)₂N(R⁴)₂;
R³ is C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{3a};
each R^{3a} is independently deuterium, halogen, —Y—CN, —Y—NO₂, —Y—OH, —Y—OR^{a}, —Y—OC(═O)R^{a}, —Y—OC(═O)OR^{b}, —Y—OC(═O)NR^{c}R^{d}, —Y—SH, —Y—SR^{a}, —Y—S(═O)R^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, —Y-cycloalkyl, —Y-heterocycloalkyl, —Y-aryl, or —Y-heteroaryl; wherein the alkyl, alkenyl, alkynyl, cycloalkyl, heterocycloalkyl, aryl, and heteroaryl is optionally substituted with one or more R^{3b};
or two R^{3a} on the same atom are taken together to form an oxo;
each R^{3b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{3b} on the same atom are taken together to form an oxo;
each R⁴ is independently hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, heterocycloalkyl, aryl, or heteroaryl; wherein the alkyl, cycloalkyl, heterocycloalkyl, aryl, and heteroaryl is optionally substituted with one or more R^{4a};
each R^{4a} is independently deuterium, halogen, —Y—CN, —Y—NO₂, —Y—OH, —Y—OR^{a}, —Y—OC(═O)R^{a}, —Y—OC(═O)OR^{b}, —Y—OC(═O)NR^{c}R^{d}, —Y—SH, —Y—SR^{a}, —Y—S(═O)R^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{4b};
or two R^{4a} on the same atom are taken together to form an oxo;
each R^{4b} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{4b} on the same atom are taken together to form an oxo;
Y is absent or C₁-C₆ alkylene optionally substituted with one or more deuterium, halogen, —CN, —OH, or —OR^{a};
Ring A is cycloalkyl, heterocycloalkyl, aryl, or heteroaryl;
L is absent or C₁-C₃ alkylene optionally substituted with one or more deuterium, halogen, —CN, —OH, or —OR^{a};
R^{6a} is —OH, —OR^{a}, or C₁-C₆alkyl;
R^{6b} is —OH, —OR^{a}, or C₁-C₆alkyl;
each R⁷ is independently deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R⁷ on the same atom are taken together to form an oxo;
n is 0-4;
each R is independently deuterium, halogen, —CN, —OH, —OR^{a}, —SH, —SR^{a}, —NR^{c}R^{d}, —NR^{c}C(═O)R^{b}, —C(═O)NR^{c}R^{d}, —C(═O)R^{a}, —C(═O)OW, C₁-C₆ alkyl, C₁-C₆ haloalkyl, C₁-C₆ hydroxyalkyl, C₁-C₆deuteroalkyl, C₁-C₆ aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or —F; or
X¹ and X² are taken together with the boron atom to which there are attached to form an optionally substituted cyclic boronate ester;
R^{X} is C₁-C₆ alkyl or cycloalkyl;
Z is hydrogen, R¹¹, —(R¹⁰)_{w}OR¹¹, —(R¹⁰)_{w}O(R¹⁰)_{w}OR¹¹, —R¹⁰OC(═O)R¹¹, —R¹⁰OC(═O)OR¹¹, —R¹⁰OC(═O)NHR¹¹, —R¹⁰OC(═O)N(R¹¹)₂, alkyloxyalkyl, acyloxyalkyl, or alkyl-[1,3]dioxol-2-one;
each R¹⁰ is independently —CH₂—, —CH(CH₃)—, —C(CH₃)₂—, or 1,1′-cyclopropylene;
each R¹¹ is independently C₁-C₆ alkyl, cycloalkyl, heterocycloalkyl, aryl, or heteroaryl; or
two R¹¹ are taken together with the nitrogen to which they are attached to form an optionally substituted heterocycloalkyl optionally substituted with C₁-C₆alkyl, C₁-C₆haloalkyl, C₁-C₆deuteroalkyl, C₁-C₆hydroxyalkyl, or C₁-C₆aminoalkyl;
w is 2-4;
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
provided that the compound is not

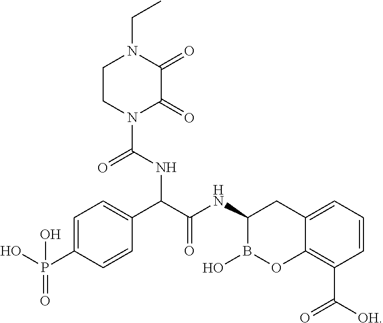 [SMILES: CCN1CCN(C(=O)NC(C(=O)N[C@H]2Cc3cccc(C(=O)O)c3OB2O)c2ccc(P(=O)(O)O)cc2)C(=O)C1=O]

2. The compound of claim 1, wherein R² is C₁-C₆ alkyl.

3. The compound of claim 1, wherein R² is —C(═O)R³ or —S(═O)₂R³.

4. The compound of claim 1, wherein R² is —C(═O)R³.

5. The compound of claim 1, wherein R² is —C(═O)N(R⁴)₂ or —S(═O)₂N(R⁴)₂.

6. The compound of any one of claims 1-4, wherein R³ is C₁-C₆alkyl, cycloalkyl, heterocycloalkyl, aryl, or heteroaryl; wherein the alkyl, cycloalkyl, heterocycloalkyl, aryl, and heteroaryl is optionally substituted with one or more R^{3a}.

7. The compound of any one of claims 1-4 or 6, wherein R³ is cycloalkyl, heterocycloalkyl, aryl, or heteroaryl; wherein the cycloalkyl, heterocycloalkyl, aryl, and heteroaryl is optionally substituted with one or more R^{3a}.

8. The compound of any one of claims 1-4 or 6 or 8, wherein R³ is heterocycloalkyl optionally substituted with one or more R^{3a}.

9. The compound of any one of claims 1-8, wherein each R^{3a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{3b}; or two R^{3a} on the same atom are taken together to form an oxo.

10. The compound of any one of claims 1-8, wherein each R^{3a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆haloalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{3b}; or two R^{3a} on the same atom are taken together to form an oxo.

11. The compound of any one of claims 1-8, wherein each R^{3a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—NR^{b}C(═O)R^{a}, —Y—NR^{b}S(═O)₂R^{a}, —Y—C(═O)R^{a}, C₁-C₆alkyl, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{3b}; or two R^{3a} on the same atom are taken together to form an oxo.

12. The compound of any one of claims 1-8, wherein each R^{3a} is independently halogen, —Y—CN, —Y—OH, —Y—OR^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—NR^{b}C(═O)R^{a}, —Y—NR^{b}S(═O)₂R^{a}, —Y—C(═O)R^{a}, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{3b}; or two R^{3a} on the same atom are taken together to form an oxo.

13. The compound of any one of claims 1-8, wherein each R^{3a} is not C₁-C₆alkyl.

14. The compound of any one of claims 1-13, wherein each R^{3b} is independently deuterium, halogen, —CN, —OH, —OR^{a}, —S(═O)₂R^{a}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R^{3b} on the same atom are taken together to form an oxo.

15. The compound of any one of claims 1-13, wherein each R^{3b} is independently deuterium, halogen, —CN, —OH, —OR^{a}, —S(═O)₂R^{a}, —C(═O)R^{a}, C₁-C₆alkyl, or C₁-C₆haloalkyl.

16. The compound of any one of claims 1-15, wherein the compound is of Formula (Ia-1) or Formula (Ib-1): 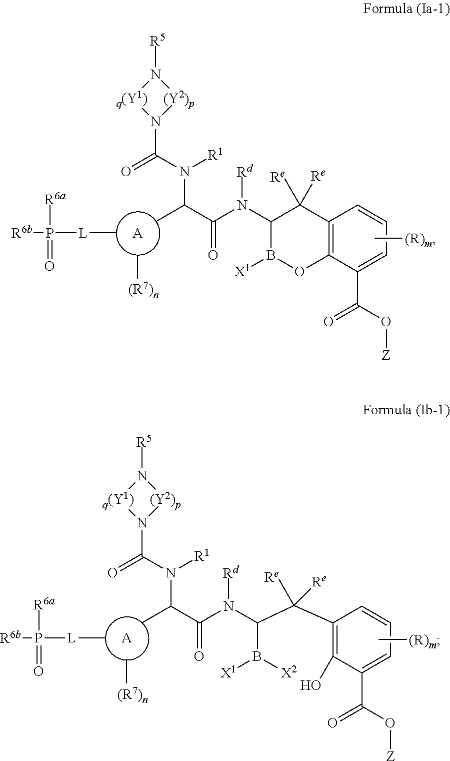
wherein:
each Y¹ and Y² is independently —C(═O)— or —C(R^{Y})₂—;
each R^{Y} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
q is 1-3;
p is 1-3;
R⁵ is —Y—OC(═O)R^{a}, —Y—OC(═O)OR^{b}, —Y—OC(═O)NR^{c}R^{d}, —Y—SH, —Y—SR^{a}, —Y—S(═O)R^{a}, —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, —Y-cycloalkyl, —Y-heterocycloalkyl, —Y-aryl, or —Y-heteroaryl; wherein the alkyl, alkenyl, alkynyl, cycloalkyl, heterocycloalkyl, aryl, and heteroaryl is optionally substituted with one or more R^{5a}; and
each R^{5a} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
or two R^{5a} on the same atom are taken together to form an oxo.

17. The compound of claim 16, wherein q is 2; each Y¹ is —C(R^{Y})₂—; p is 2; and each Y² is —C(═O)—.

18. The compound of claim 16, wherein q is 2; each Y¹ is —C(R^{Y})₂—; p is 1; and Y² is —C(═O)—.

19. The compound of claim 16, wherein q is 2; each Y¹ is —C(R^{Y})₂—; p is 2; and one Y² is —C(R^{Y})₂— and one Y² is —C(═O)—.

20. The compound of claim 16, wherein q is 2; one Y¹ is —C(R⁵)₂— and one Y¹ is —C(═O)—; p is 2; and one Y² is —C(R⁵)₂— and one Y² is —C(═O)—.

21. The compound of any one of claims 16-20, wherein each R^{Y} is independently hydrogen, halogen, or C₁-C₆alkyl.

22. The compound of any one of claims 16-21, wherein each R^{Y} is hydrogen.

23. The compound of any one of claims 16-22, wherein

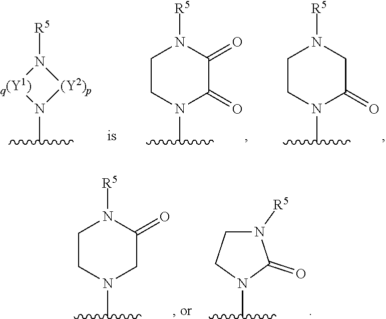

24. The compound of any one of claims 16-23, wherein

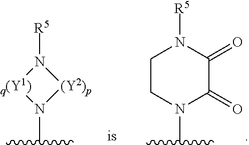

25. The compound of any one of claims 16-23, wherein

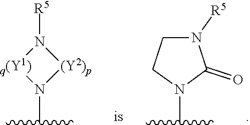

26. The compound of any one of claims 16-25, wherein R is —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{5a}.

27. The compound of any one of claims 16-25, wherein R⁵ is —Y—S(═O)₂R^{a}, —Y—S(═O)₂NR^{c}R^{d}, —Y—NR^{c}R^{d}, —Y—NR^{b}C(═O)NR^{c}R^{d}, —Y—S(═O)₂(C₁-C₆ alkylene)NR^{b}C(═O)NR^{c}R^{d}, —Y—NR^{b}C(═O)R^{a}, —Y—S(═O)₂(C₁-C₆ alkylene)NR^{b}C(═O)R^{a}, —Y—NR^{b}C(═O)OR^{b}, —Y—NR^{b}S(═O)₂R^{a}, —Y—S(═O)₂(C₁-C₆ alkylene)NR^{b}S(═O)₂R^{a}, —Y—NR^{b}S(═O)₂NR^{c}R^{d}, —Y—S(═O)₂(C₁-C₆ alkylene)NR^{b}S(═O)₂NR^{c}R^{d}, —Y—C(═O)R^{a}, —Y—C(═O)OR^{b}, —Y—C(═O)NR^{c}R^{d}, C₁-C₆haloalkyl, C₁-C₆deuteroalkyl, C₁-C₆hydroxyalkyl, C₁-C₆aminoalkyl, C₁-C₆heteroalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{5a}.

28. The compound of any one of claims 16-25, wherein R is —Y—S(═O)₂R^{a}, —Y—S(═O)₂NR^{c}R^{d}, —Y—NR^{c}R^{d}, —Y—NR^{b}C(═O)R^{a}, —Y—NR^{b}S(═O)₂R^{a}, C₁-C₆alkyl, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{5a}.

29. The compound of any one of claims 16-25, wherein R is —Y—S(═O)₂R^{a}, —Y—S(═O)₂NR^{c}R^{d}, —Y—NR^{c}R^{d}, —Y—NR^{b}C(═O)R^{a}, —Y—NR^{b}S(═O)₂R^{a}, C₁-C₆haloalkyl, C₁-C₆aminoalkyl, —Y-cycloalkyl, —Y-heterocycloalkyl, —Y-aryl, or —Y-heteroaryl; wherein the alkyl, cycloalkyl, heterocycloalkyl, aryl, and heteroaryl is optionally substituted with one or more R^{5a}.

30. The compound of any one of claims 16-25, wherein each R⁵ is not C₁-C₆alkyl.

31. The compound of any one of claims 16-30, wherein each R^{5a} is independently deuterium, halogen, —CN, —OH, —OR^{a}, —S(═O)₂R^{a}, —C(═O)R^{a}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, cycloalkyl, or heterocycloalkyl; or two R^{3b} on the same atom are taken together to form an oxo.

32. The compound of any one of claims 16-31, wherein each R^{5a} is independently deuterium, halogen, —CN, —OH, —OR^{a}, —S(═O)₂R^{a}, —C(═O)R^{a}, C₁-C₆alkyl, or C₁-C₆haloalkyl.

33. The compound of any one of claims 1-32, wherein Ring A is aryl or heteroaryl.

34. The compound of any one of claims 1-33, wherein Ring A is aryl.

35. The compound of any one of claims 1-34, wherein L is absent.

36. The compound of any one of claims 1-34, wherein L is C₁-C₃ alkylene optionally substituted with one or more deuterium, halogen, —CN, —OH, or —OR^{a}.

37. The compound of any one of claims 1-34, wherein L is C₁ alkylene substituted with one or more halogen.

38. The compound of any one of claims 1-37, wherein R^{6a} is —OH or C₁-C₆alkyl.

39. The compound of any one of claims 1-38, wherein R^{6a} is —OH.

40. The compound of any one of claims 1-39, wherein R^{6b} is —OH or C₁-C₆alkyl.

41. The compound of any one of claims 1-40, wherein R^{6b} is —OH.

42. The compound of any one of claims 1-41, wherein each R⁷ is independently deuterium, halogen, —CN, —OH, —OR^{a}, —NR^{c}R^{d}, C₁-C₆alkyl, or C₁-C₆haloalkyl.

43. The compound of any one of claims 1-42, wherein each R⁷ is independently halogen or —OH.

44. The compound of any one of claims 1-43, wherein n is 1 or 2.

45. The compound of any one of claims 1-43, wherein n is 0 or 1.

46. A compound of Formula (IIa) or (IIb), or a pharmaceutically acceptable salt, solvate, stereoisomer, tautomer, N-oxide, dimer, or trimer thereof: 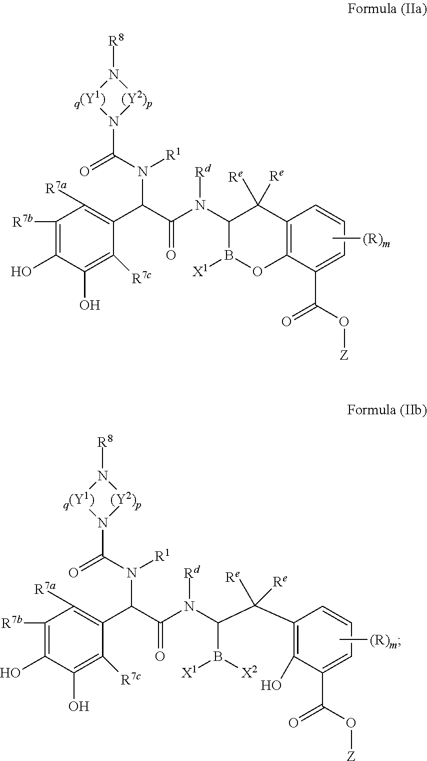
wherein:
R¹ is hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each Y¹ and Y² is independently —C(═O)— or —C(R^{Y})₂—;
each R^{Y} is independently hydrogen, deuterium, halogen, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
q is 1-3;
p is 1-3;
R⁸ is —(W)ᵤ—Ring B;
each W is independently —C(R^{W1})₂—, —O—, —NR^{W2}—, —S—, —S(═O)—, —S(═O)₂—, or —C(═O)—;
each R^{W1} is independently hydrogen, deuterium, halogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{W2} is independently hydrogen, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or one R^{W1} and one R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{W1}, when present, are taken together to form a cycloalkyl or heterocycloalkyl; each optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or two R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
u is 1-10;
Ring B is cycloalkyl, heterocycloalkyl, aryl, or heteroaryl; each optionally substituted with one or more R^{B};
each R^{B} is independently deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —NR^{b}S(═O)₂NR^{c}R^{d}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl;
R^{7a} is hydrogen, deuterium, halogen, —OH, —OR^{a}, —NR^{c}R^{d}, or C₁-C₆alkyl;
R^{7b} is deuterium, halogen, —OH, —OR^{a}, —NR^{c}R^{d}, or C₁-C₆alkyl;
R^{7c} is deuterium, halogen, —OH, —OR^{a}, —NR^{c}R^{d}, or C₁-C₆alkyl;
each R is independently deuterium, halogen, —CN, —OH, —OR^{a}, —SH, —SR^{a}, —NR^{c}R^{d}, —NR^{c}C(═O)R^{b}, —C(═O)NR^{c}R^{d}, —C(═O)R^{a}, —C(═O)OR^{a}, C₁-C₆ alkyl, C₁-C₆ haloalkyl, C₁-C₆deuteroalkyl, C₁-C₆ hydroxyalkyl, C₁-C₆ aminoalkyl, —Y-cycloalkyl, —Y-heterocycloalkyl, —Y-aryl, or —Y-heteroaryl; wherein each alkyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
m is 0-3;
R^{d} is hydrogen or C₁-C₆ alkyl;
each R^{e} is independently hydrogen, —CN, —OH, C₁-C₆ alkyl, or cycloalkyl;
X¹ and X² are independently —OH, —OR^{X}, or —F; or
X¹ and X² are taken together with the boron atom to which there are attached to form an optionally substituted cyclic boronate ester;
R^{X} is C₁-C₆ alkyl or cycloalkyl;
Z is hydrogen, R¹¹, —(R¹⁰)_{w}OR¹¹, —(R¹⁰)_{w}O(R¹⁰)_{w}OR¹¹, —R¹⁰OC(═O)R¹¹, —R¹⁰OC(═O)OR¹¹, —R¹⁰OC(═O)NHR¹¹, —R¹⁰OC(═O)N(R¹¹)₂, alkyloxyalkyl, acyloxyalkyl, or alkyl-[1,3]dioxol-2-one;
each R¹⁰ is independently —CH₂—, —CH(CH₃)—, —C(CH₃)₂—, or 1,1′-cyclopropylene;
each R¹¹ is independently C₁-C₆ alkyl, cycloalkyl, heterocycloalkyl, aryl, or heteroaryl; or
two R¹¹ are taken together with the nitrogen to which they are attached to form an optionally substituted heterocycloalkyl optionally substituted with C₁-C₆alkyl, C₁-C₆haloalkyl, C₁-C₆deuteroalkyl, C₁-C₆hydroxyalkyl, or C₁-C₆aminoalkyl;
w is 2-4;
Y is absent or C₁-C₆ alkylene optionally substituted with one or more deuterium, halogen, —CN, —OH, or —OR^{a};
each R^{a} is independently C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
each R^{b} is independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl; and
each R^{c} and R^{d} are independently hydrogen, C₁-C₆alkyl, C₁-C₆haloalkyl, C₁-C₆deuteroalkyl, C₁-C₆hydroxyalkyl, C₁-C₆aminoalkyl, C₂-C₆alkenyl, C₂-C₆alkynyl, —Y-cycloalkyl, —Y-heterocycloalkyl, —Y-aryl, or —Y-heteroaryl; wherein each alkyl, alkenyl, alkynyl, cycloalkyl, heterocycloalkyl, aryl, and heteroaryl is independently optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
or R^{c} and R^{d} are taken together with the atom to which they are attached to form a heterocycloalkyl optionally substituted with one or more oxo, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, —C(═O)NH₂, —C(═O)NHCH₃, —C(═O)N(CH₃)₂, C₁-C₆alkyl, C₁-C₆haloalkyl, C₁-C₆deuteroalkyl, C₁-C₆hydroxyalkyl, or C₁-C₆aminoalkyl;
provided that the compound is not

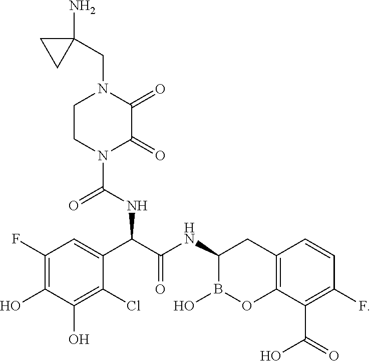 [SMILES: NC1(CN2CCN(C(=O)N[C@@H](C(=O)N[C@H]3Cc4ccc(F)c(C(=O)O)c4OB3O)c3cc(F)c(O)c(O)c3Cl)C(=O)C2=O)CC1]

47. The compound of claim 46, wherein q is 2; each Y¹ is —C(R^{Y})₂—; p is 2; and each Y² is —C(═O)—.

48. The compound of claim 46, wherein q is 2; each Y¹ is —C(R^{Y})₂—; p is 1; and Y² is —C(═O)—.

49. The compound of claim 46, wherein q is 2; each Y¹ is —C(R^{Y})₂—; p is 2; and one Y² is —C(R)₂— and one Y² is —C(═O)—.

50. The compound of claim 46, wherein q is 2; one Y¹ is —C(R⁵)₂— and one Y¹ is —C(═O)—; p is 2; and one Y² is —C(R⁵)₂— and one Y² is —C(═O)—.

51. The compound of any one of claims 46-50, wherein each R¹ is independently hydrogen, halogen, or C₁-C₆alkyl.

52. The compound of any one of claims 46-51, wherein each R^{Y} is hydrogen.

53. The compound of any one of claims 46-52, wherein

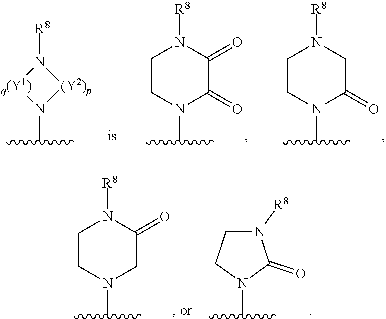

54. The compound of any one of claims 46-53, wherein

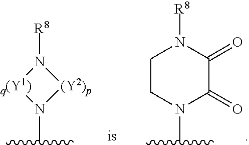

55. The compound of any one of claims 46-53, wherein

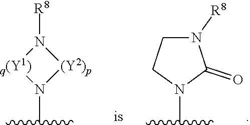

56. The compound of any one of claims 46-55, wherein R^{7a} is hydrogen or halogen.

57. The compound of any one of claims 46-56, wherein R^{7a} is hydrogen.

58. The compound of any one of claims 46-57, wherein R^{7b} is halogen.

59. The compound of any one of claims 46-58, wherein R^{7c} is halogen.

60. The compound of any one of claims 46-59, wherein u is 1-8.

61. The compound of any one of claims 46-59, wherein u is 1-7.

62. The compound of any one of claims 46-59, wherein u is 1-6.

63. The compound of any one of claims 46-59, wherein u is 1-5.

64. The compound of any one of claims 46-59, wherein u is 3-8.

65. The compound of any one of claims 46-59, wherein u is 4-8.

66. The compound of any one of claims 46-59, wherein u is 5-8.

67. The compound of any one of claims 46-59, wherein u is 4-6.

68. The compound of any one of claims 46-67, wherein —(W)ᵤ— is —[C(R^{W1})₂]₁₋₄—NR^{W2}—C(═O), —[C(R^{W1})₂]₁₋₄—NR^{W2}—C(═O)—[C(R^{W1})₂]₁₋₂—, —[C(R^{W1})₂]₁₋₄—NR^{W2}—S(═O)₂—, —[C(R^{W1})₂]₁₋₄—NR^{W2}—C(═O)—NR^{W2}—, —[C(R^{W1})₂]₁₋₃—NR^{W2}—[C(R^{W1})₂]₁₋₂—, —[C(R^{W1})₂]₁₋₄—, —S(═O)₂—[C(R^{W1})₂]₁₋₄—NR^{W2}—C(═O)—, or —S(═O)₂—[C(R^{W1})₂]₁₋₄—NR^{W2}—C(═O)—NR^{W2}—.

69. The compound of any one of claims 46-68, wherein —(W)ᵤ— is —[C(R^{W1})₂]₁₋₄—NR^{W2}—C(═O)—.

70. The compound of any one of claims 46-68, wherein —(W)ᵤ— is —[C(R^{W1})₂]₁₋₄—NR^{W2}—C(═O)—[C(R^{W1})₂]₁₋₂—.

71. The compound of any one of claims 46-68, wherein —(W)ᵤ— is —[C(R^{W1})₂]₁₋₄—NR^{W2}—S(═O)₂—.

72. The compound of any one of claims 46-68, wherein —(W)ᵤ— is —[C(R^{W1})₂]₁₋₄—NR^{W2}—C(═O)—NR^{W2}—.

73. The compound of any one of claims 46-68, wherein —(W)ᵤ— is —[C(R^{W1})₂]₁₋₃—NR^{W2}—[C(R^{W1})₂]₁₋₃—.

74. The compound of any one of claims 46-68, wherein —(W)ᵤ— is —[C(R^{W1})₂]₁₋₄—.

75. The compound of any one of claims 46-68, wherein —(W)ᵤ— is —S(═O)₂—[C(R^{W1})₂]₁₋₄—NR^{W2}—C(═O)—.

76. The compound of any one of claims 46-68, wherein —(W)ᵤ— is S(═O)₂—[C(R^{W1})₂]₁₋₄—NR^{W2}—C(═O)—NR^{W2}—.

77. The compound of any one of claims 46-76, wherein each R^{W1} is independently hydrogen, deuterium, halogen, C₁-C₆alkyl, or C₁-C₆haloalkyl.

78. The compound of any one of claims 46-77, wherein each R^{W1} is independently hydrogen or C₁-C₆alkyl.

79. The compound of any one of claims 46-78, wherein each R^{W1} is hydrogen.

80. The compound of any one of claims 46-79, wherein each R^{W2} is independently hydrogen, C₁-C₆alkyl, or C₁-C₆haloalkyl.

81. The compound of any one of claims 46-80, wherein each R^{W2} is independently hydrogen or C₁-C₆alkyl.

82. The compound of any one of claims 46-81, wherein each R^{W2} is hydrogen.

83. The compound of any one of claims 46-81, wherein each R^{W2} is independently C₁-C₆alkyl.

84. The compound of any one of claims 46-76, wherein one R^{W1} and one R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

85. The compound of any one of claims 46-76, wherein two R^{W1}, when present, are taken together to form a cycloalkyl or heterocycloalkyl; each optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

86. The compound of any one of claims 46-76, wherein two R^{W2}, when present, are taken together to form a heterocycloalkyl optionally substituted with one or more C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

87. The compound of any one of claims 46-86, wherein Ring B is heterocycloalkyl, aryl, or heteroaryl; each optionally substituted with one or more R^{B}.

88. The compound of any one of claims 46-87, wherein Ring B is aryl or heteroaryl; each optionally substituted with one or more R^{B}.

89. The compound of any one of claims 46-88, wherein Ring B is aryl optionally substituted with one or more R^{B}.

90. The compound of any one of claims 46-88, wherein Ring B is heteroaryl optionally substituted with one or more R^{B}.

91. The compound of any one of claims 46-90, wherein each R^{B} is independently deuterium, halogen, —CN, —OH, —OR^{a}, —S(═O)R^{a}, —S(═O)₂R^{a}, —S(═O)₂NR^{c}R^{d}, —NR^{c}R^{d}, —C(═O)R^{a}, —C(═O)OR, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

92. The compound of any one of claims 46-91, wherein each R^{B} is independently deuterium, halogen, —CN, —OH, —OR^{a}, —NR^{c}R^{d}, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl.

93. The compound of any one of claims 46-92, wherein each R^{B} is independently halogen, —CN, —OH, —OR^{a}, —NR^{c}R^{d}, C₁-C₆alkyl, or C₁-C₆haloalkyl.

94. The compound of any one of claims 46-93, wherein each R^{B} is independently halogen or OH.

95. The compound of any one of claims 1-94, wherein R¹ is hydrogen or C₁-C₆alkyl.

96. The compound of any one of claims 1-95, wherein R¹ is hydrogen.

97. The compound of any one of claims 1-96, wherein R^{d} is hydrogen.

98. The compound of any one of claims 1-97, wherein each R^{c} is hydrogen.

99. The compound of any one of claims 1-98, wherein X¹ and X² are —OH.

100. The compound of any one of claims 1-99, wherein each R is independently deuterium, halogen, —CN, —OH, —OR^{a}, —NR^{c}R^{d}, —C(═O)R^{a}, —C(═O)OR^{a}, C₁-C₆ alkyl, C₁-C₆ haloalkyl, C₁-C₆deuteroalkyl, C₁-C₆ hydroxyalkyl, or C₁-C₆ aminoalkyl.

101. The compound of any one of claims 1-100, wherein each R is independently deuterium, halogen, —CN, —OH, —OR^{a}, —NR^{c}R^{d}, C₁-C₆ alkyl, or C₁-C₆ haloalkyl.

102. The compound of any one of claims 1-101, wherein each R is independently deuterium, halogen, C₁-C₆ alkyl, or C₁-C₆ haloalkyl.

103. The compound of any one of claims 1-102, wherein each R is independently halogen.

104. The compound of any one of claims 1-103, wherein m is 0 or 1.

105. The compound of any one of claims 1-103, wherein m is 1 or 2.

106. The compound of any one of claims 1-105, wherein Z is hydrogen.

107. The compound of any one of claims 1-105, wherein Z is R¹¹; and R¹¹ is C₁-C₆ alkyl.

108. The compound of any one of claims 1-105, wherein Z is —R¹⁰OC(═O)R¹¹ or —R¹⁰OC(═O)OR¹¹; R¹⁰ is —CH₂— or —CH(CH₃)—; and R¹¹ is alkyl, cycloalkyl, or heterocycloalkyl.

109. The compound of any one of claims 1-108, wherein Y is absent.

110. The compound of any one of claims 1-108, wherein Y is C₁-C₆ alkylene optionally substituted with one or more deuterium, halogen, —CN, —OH, or —OR^{a}.

111. The compound of any one of claims 1-108, wherein Y is C₁-C₆ alkylene.

112. The compound of any one of claims 1-108, wherein Y is C₁-C₄ alkylene.

113. The compound of any one of claims 1-108, wherein Y is C₂-C₆ alkylene.

114. The compound of any one of claims 1-108, wherein Y is C₂-C₄ alkylene.

115. The compound of any one of claims 1-108, wherein Y is C₁ alkylene.

116. A compound selected from a compound of table 1, table 2, table 3, or table 4, or a pharmaceutically acceptable salt, solvate, stereoisomer, tautomer, N-oxide, dimer, or trimer thereof.

117. A pharmaceutical composition comprising the compound of any one of claims 1-116, or a pharmaceutically acceptable salt, solvate, stereoisomer, tautomer, N-oxide, dimer, or trimer thereof, and a pharmaceutically acceptable excipient.

118. A method of treating a bacterial infection in a subject, comprising administering to the subject an effective amount of the compound of any one of claims 1-116, or a pharmaceutically acceptable salt, solvate, stereoisomer, tautomer, N-oxide, dimer, or trimer thereof, or the pharmaceutical composition of claim 117.

119. A method of inhibiting a bacterial penicillin-binding protein in a human infected with a bacterial infection, comprising contacting said bacterial penicillin-binding protein with an effective amount of the compound of any one of claims 1-116, or a pharmaceutically acceptable salt, solvate, stereoisomer, tautomer, N-oxide, dimer, or trimer thereof, or the pharmaceutical composition of claim 117.

120. The method of claim 118 or 119, wherein the bacterial infection is caused by Neisseria gonorrhoeae.

121. The method of claim 118 or 119, wherein the bacterial infection is caused by Burkholderia pseudomallei.

122. The method of claim 118 or 119, wherein the bacterial infection is caused by Pseudomonas aeruginosa.

123. The method of claim 118 or 119, wherein the bacterial infection is caused by Acinetobacter baumannii.

124. The method of claim 118 or 119, wherein the bacterial infection is caused by Pseudomonas aeruginosa/Acinetobacter baumannii.

125. The method of claim 118 or 119, wherein the bacterial infection is caused by a carbapenem-resistant enterobacterales (CRE).
